(19) United States
(12) Patent Application Publication
(10) Pub. No.: US 2025/0127887 A1
(43) Pub. Date: Apr. 24, 2025

(54) Self-Assembling Nanoparticles Based On Amphiphilic Peptides

(71) Applicants: Barinthus Biotherapeutics North America, Inc, Germantown, MD (US); The United States of America,as represented by the Secretary,Department of Health and Human Services, Bethesda, MD (US)

(72) Inventors: Geoffrey M. Lynn, McLean, VA (US); Sarah R. Nichols, Mount Airy, MD (US); Vincent L. Coble, Mount Airy, MD (US); Andrew S. Ishizuka, Brookline, MA (US); Hugh Clarke Welles, Baltimore, MD (US); Robert N. Goddu, Colombia, MD (US); Christopher Martin O’Brien Garliss, Baltimore, MD (US); Ramiro Andrei Ramirez-Valdez, Roxbury, MA (US)

(73) Assignees: Barinthus Biotherapeutics North America, Inc., Germantown, MD (US); The United States of America,as represented by the Secretary,Department of Health and Human Services, Bethesda, MD (US)

(21) Appl. No.: 18/277,220

(22) PCT Filed: Feb. 16, 2022
(86) PCT No.: PCT/US2022/016600

§ 371 Date: Aug. 14, 2023

Related U.S. Application Data
(60) Provisional application No. 63/197,092, filed on Jun. 4, 2021.
(60) Provisional application No. 63/149,996, filed on Feb. 16, 2021.

(51) Int. Cl. A61K 39/385; A61K 39/00; A61P 37/04

(52) U.S. Cl. CPC A61K 39/385; A61K 39/0008; A61K 39/001102; A61P 37/04; A61K 2039/6031

(57) ABSTRACT

The present disclosure relates to a vaccine comprising an amphiphile having the formula S-[B]-[U]-H and at least one peptide antigen conjugate having the formula selected from [S]-[E1]-A-[E2]-[U]-H and H-[U]-[E1]-A-[E2]-[S], wherein the amphiphile and/or the at least one peptide antigen conjugate comprises a dendron amplifier. The vaccine is useful in treating or preventing a cancer, an autoimmune disease, an allergy, an infectious disease, a cardiovascular disease, or a neurodegenerative disease.

### CROSS-REFERENCE TO RELATED APPLICATIONS

This application is a national phase entry pursuant to 35 U.S.C. § 371 of International Application No. PCT/US2022/016600, filed Feb. 16, 2022, which claims the benefit of priority to U.S. Provisional Patent Application Nos. 63/197,092, filed Jun. 4, 2021 and 63/149,996, filed Feb. 16, 2021, the contents of each of which are hereby incorporated by reference in their entirety.

### GOVERNMENT RIGHTS

This invention was created in the performance of a Cooperative Research and Development Agreement with the National Institutes of Health, an Agency of the Department of Health and Human Services. The Government of the United States has certain rights in this invention.

### SEQUENCE LISTING

The instant application contains a Sequence Listing which has been submitted electronically in ASCII format and is hereby incorporated by reference in its entirety. Said ASCII copy, created on Dec. 27, 2024, is named BARI_010WOUS_SL.txt, and is 186.9 kilobytes in size.

### TECHNICAL FIELD

The present disclosure relates to novel amphiphile compositions, particularly peptide amphiphile compositions, that can be used to form nanoparticles, including micelle structures or polymersomes, methods of manufacturing the amphiphile compositions, processes for formulating drug molecules with the amphiphile compositions that form nanoparticles, and therapeutic uses of the nanoparticles for drug delivery.

### BACKGROUND

Vaccines can be used to generate antibody and/or T cell responses against any pathogen or molecule(s) associated with disease as a means to prevent disease onset or reduce disease severity. The choice of vaccination platform utilized, in part, depends on the target pathogen or molecule(s) and the type of immune response needed.

Peptide-based vaccines comprise synthetic, peptide antigens derived from infectious organisms, toxins, or any proteins associated with pathology (e.g., prions, misfolded proteins, etc.) that are used to induce antibody and T cell responses against the peptide antigen that cross-react with the native protein present on the infectious organism, toxin or types of proteins associated with disease. As peptides alone are not immunogenic, peptide antigens are typically formulated with an immunostimulant and/or delivery vehicle, referred to as vaccine adjuvants. A major advantage of peptide-based vaccines is that small fragments of proteins derived from pathogens or disease-causing proteins can be produced synthetically, including with post-translational modifications (e.g., glycans, phosphate groups, etc.), and used as antigens for inducing highly focused immune responses against conserved structures (i.e., peptide sequences and any higher order structures) of pathogens, toxins and disease-causing proteins. The benefit of focusing the immune responses narrowly against specific sites of interest is that this avoids off-target antibody and T cell responses that can dilute the immune response and potentially lead to pathology.

Based on these characteristics, peptide-based vaccines are particularly well-suited for focusing T cell responses against tumor antigens for preventing or treating cancer. Accordingly, tumor-derived peptide antigens can be used to focus immune responses against cancer cells while avoiding immune responses directed against healthy tissue. Similarly, peptide-based vaccines are also particularly well-suited for generating antibody responses against proteins associated with cardiovascular disease, neurodegenerative disease and other pathologies associated with aging. The benefit of peptide-based vaccines is that antibody responses can be directed against only a small portion of a target protein or only the aberrant version of a protein, e.g., misfolded protein or abnormal protein that is a product of a splice variant, without inducing off-target antibodies.

Another application of peptide-based vaccines is for inducing neutralizing antibodies against infectious organisms, such as HIV, malaria, SARS and other coronaviruses, where highly focused antibody responses against conserved sites of neutralization may be required for preventing infection.

Additionally, peptide-based vaccines alone can be administered alone or combined with immunosuppressants to induce tolerance for the treatment of allergies and autoimmunity.

Numerous peptide-based vaccine technologies have been developed, though, a major challenge is ensuring consistent manufacturing of uniform formulations given the tremendous physicochemical heterogeneity of peptide antigens. Therefore, improved formulation and delivery approaches are needed to ensure consistent formulations with any peptide antigen.

Another challenge is for focusing antibody responses against minimal immunogens that represent fragments of full-length protein antigens. Such approaches may be needed for the development of successful prophylactic vaccination strategies directed against certain viruses that are highly variable and thus may require focused antibody induction against conserved sites. Minimal immunogen vaccines may also be beneficial for treating certain neurodegenerative diseases, cardiovascular disease and cancers, where focused antibody responses may be required for balancing safety and efficacy. A current challenge, however, is that most vaccine platforms, e.g., whole organism, expression systems (e.g., DNA and viruses), and protein subunit vaccines lead to broad antibody responses and are not well-suited for focusing antibodies against minimal immunogens, whereas the most commonly used minimal immunogen vaccine platforms, i.e., KLH and virus like particles, rely on conjugation of synthetic peptides to recombinant carriers, which often results in formulation variability as well as increased costs due to the use of recombinant manufacturing processes. Therefore, alternative, preferably fully synthetic systems for displaying minimal immunogens are needed.

Finally, while there has been increasing interest in use of peptide-based vaccines, for inducing tolerance for treating allergies and autoimmunity, currently used PLGA particles and liposomes rely on an empirical formulation process that is not well suited for generating particles formulations with multiple peptide antigens and immunomodulators that may be required. Moreover, the optimal combination of peptide antigens and immunomodulators is unknown. Therefore, there is a need for improved methods of delivering multiple immunomodulators and peptide antigens in particles for inducing tolerance for the treatment of allergies and autoimmunity.

Discussed herein are improved compositions and methods of manufacturing vaccines that address the aforementioned challenges.

### SUMMARY

The present disclosure relates to a vaccine comprising an amphiphile having the formula S-[B]-[U]-H; and at least one peptide antigen conjugate having the formula selected from [S]-[E1]-A-[E2]-[U]-H and H-[U]-[E1]-A-[E2]-[S],
wherein S, independently for each occurrence, is a solubilizing block;
B is a spacer;
H, independently for each occurrence, is a hydrophobic block,
   wherein one or more drug molecules (D) are optionally attached to each H directly or via a suitable linker X1;
   A, independently for each occurrence, is a peptide antigen;
   E1, independently for each occurrence, is an N-terminal extension;
   E2, independently for each occurrence, is a C-terminal extension;
   U, independently for each occurrence, is a linker;
   [ ] denotes that the group is optional; and
   — denotes that the two adjacent groups are directly attached to one another by a covalent bond or indirectly to one another via a suitable linker X,
   wherein the amphiphile and/or the at least one peptide antigen conjugate comprises a dendron amplifier.

The present disclosure also relates to a vaccine for inducing tolerance comprising an amphiphile having the formula S-[B]-[U]-H; and at least one peptide antigen conjugate having the formula selected from [S]-[E1]-A-[E2]-[U]-H and H-[U]-[E1]-A-[E2]-[S],
wherein S, independently for each occurrence, is a solubilizing block;
B is a spacer;
H, independently for each occurrence, is a hydrophobic block,
   wherein one or more drug molecules (D) are optionally attached to each H directly or via a suitable linker X1;
A, independently for each occurrence, is a peptide antigen;
E1, independently for each occurrence, is an N-terminal extension;
E2, independently for each occurrence, is a C-terminal extension;
U, independently for each occurrence, is a linker;
[ ] denotes that the group is optional; and
— denotes that the two adjacent groups are directly attached to one another by a covalent
bond or indirectly to one another via a suitable linker X,
wherein the amphiphile and/or the at least one peptide antigen conjugate comprises a dendron amplifier and
at least one peptide antigen is selected from an autoantigen, alloantigen and allergen.

The present disclosure also relates to a vaccine for inducing tolerance comprising an amphiphile having the formula S-[B]-[U]-H; and at least one peptide antigen conjugate having the formula selected from [S]-[E1]-A-[E2]-[U]-H and H-[U]-[E1]-A-[E2]-[S],
wherein S, independently for each occurrence, is a solubilizing block;
B is a spacer;
H, independently for each occurrence, is a hydrophobic block,
   wherein one or more drug molecules (D) are optionally attached to each H directly or via a suitable linker X1;
A, independently for each occurrence, is a peptide antigen;
E1, independently for each occurrence, is an N-terminal extension;
E2, independently for each occurrence, is a C-terminal extension;
U, independently for each occurrence, is a linker;
[ ] denotes that the group is optional; and
— denotes that the two adjacent groups are directly attached to one another by a covalent bond or indirectly to one another via a suitable linker X,
wherein the amphiphile and/or the at least one peptide antigen conjugate comprises a dendron amplifier and
at least one A comprises a sequence wherein one or more cysteine residues have been replaced with alpha amino-butyric acid and/or one or more methionine residues have been replaced with norleucine.

The present disclosure also relates to a vaccine comprising at least one peptide antigen (A), wherein at least one A comprises a sequence wherein one or more cysteine residues have been replaced with alpha amino-butyric acid and/or one or more methionine residues have been replaced with norleucine.

The present disclosure also relates to a vaccine comprising at least one peptide antigen conjugate having the formula selected from [S]-[E1]-A-[E2]-[U]-H and H-[U]-[E1]-A-[E2]-[S],
wherein S, independently for each occurrence, is a solubilizing block;
H, independently for each occurrence, is a hydrophobic block,
   wherein one or more drug molecules (D) are optionally attached to each H directly or via a suitable linker X1;
A, independently for each occurrence, is a peptide antigen;
E1, independently for each occurrence, is an N-terminal extension;
E2, independently for each occurrence, is a C-terminal extension;
U, independently for each occurrence, is a linker;
[ ] denotes that the group is optional; and
— denotes that the two adjacent groups are directly attached to one another by a covalent bond or indirectly to one another via a suitable linker X,
wherein at least one peptide antigen is selected from an autoantigen, alloantigen and allergen and at least one drug molecule D is present and selected from an ATP-competitive mTOR inhibitor;
or wherein at least one peptide antigen is a tumor antigen or infectious disease antigen and at least one drug molecule D is present and selected from agonists of TLR-3, TLR-7, TLR-8, TLR-7/8 and STING and a second drug molecule (D2) is present and selected from inhibitors of mTORC1.

In certain embodiments of the above vaccine, at least one A comprises a sequence wherein one or more cysteine residues have been replaced with alpha amino-butyric acid and/or one or more methionine residues have been replaced with norleucine.

The present disclosure also relates to a vaccine comprising at least one peptide antigen (A), wherein at least one peptide antigen (A) comprises a sequence wherein one or more cysteine residues have been replaced with alpha amino-butyric acid and/or one or more methionine residues have been replaced with norleucine.

The present disclosure also relates to a vaccine comprising at least one peptide antigen conjugate having the formula selected from [S]-[E1]-A-[E2]-[U]-H and H-[U]-[E1]-A-[E2]-[S], wherein S, independently for each occurrence, is a solubilizing block;
H, independently for each occurrence, is a hydrophobic block,
   wherein one or more drug molecules (D) are optionally attached to each H directly or via a suitable linker X1;
A, independently for each occurrence, is a peptide antigen;
E1, independently for each occurrence, is an N-terminal extension;
E2, independently for each occurrence, is a C-terminal extension;
U, independently for each occurrence, is a linker;
wherein either:
   (i) at least one A comprises alpha amino-butyric acid and/or norleucine;
   (ii) at least one A is selected from tumor antigens, at least one D is present and is selected from agonists of TLR-7/8, and the vaccine further comprises a second drug molecule (D2) selected from inhibitors of mTOR;
   (iii) at least one A is a glycopeptide; or
   (iv) at least one A is selected from autoantigens, allergens and alloantigens and at least one D is present and is selected from ATP-competitive mTOR inhibitors;
[ ] denotes that the group is optional; and
— denotes that the two adjacent groups are directly attached to one another by a covalent bond or indirectly to one another via a suitable linker X.

The present disclosure also relates to a vaccine comprising an expression system comprising DNA or RNA encoding for at least one peptide antigen (A), wherein the vaccine further comprises at least one drug molecule (D) selected from Treg promoting immunomodulators.

The present disclosure also relates to a peptide antigen conjugate having the formula selected from S-[E1]-A-[E2]-[U]-H-[D] and [D]-H-[U]-[E1]-A-[E2]-S or a peptide antigen fragment having the formula selected from S-[E1]-A-[E2]-[U1] and [U1]-[E1]-A-[E2]-S
wherein S is a solubilizing block;
H is a hydrophobic block,
   wherein one or more drug molecules (D) are optionally attached to each H directly or via a suitable linker X1;
A is a peptide antigen;
E1 is an N-terminal extension;
E2 is a C-terminal extension;
U is a linker;
U1 is a linker precursor;
[ ] denotes that the group is optional; and
— denotes that the two adjacent groups are directly attached to one another by a covalent bond or indirectly to one another via a suitable linker X, and
wherein S comprises one or more amino acids.

The present disclosure also relates to a method of inducing an immune response in a subject in need thereof, comprising administering to the subject at least one dose of a first vaccine (V1) followed by at least one dose of a second vaccine (V2), wherein V1 is a vaccine disclosed herein; and V2 is a viral vaccine.

### BRIEF DESCRIPTION OF FIGURES

FIG. 1 shows the effect that varying (i) net charge (ii), spacer composition (PEG versus peptide), (iii) spacer length and (iv) hydrophobic block length have on the hydrodynamic behavior of linear amphiphiles of formula S-B-U-H. The data show that increasing net charge promotes nanoparticle micellization; amphiphiles with spacers, B, comprising ethylene oxide (PEG) are less prone to formation of aggregates and/or supramolecular associates as compared with amphiphiles with spacers comprising amino acids; and the net charge required to induce micellization with linear amphiphiles increases with increasing length and/or hydrophobic surface area of the hydrophobic block (H).

FIG. 2 shows cartoon schematics of amphiphilic carriers of formula S-B-[U]-H-D having either (A) linear, (B) dendron or (C) brush architecture.

FIG. 3 shows the effect that varying solubilizing block functional group (i) composition, (ii) number and (iii) net charge have on the hydrodynamic behavior of amphiphiles of formula S-B-U-H-D having either linear, dendron or brush architecture. The groups NH₂, COOH, OH and Man refer to primary amines, carboxylic acids, hydroxyl and mannose groups, respectively. Particle size was assessed by dynamic light scattering and the number mean particle size in diameter (d, nm) is reported.

FIG. 4 shows a cartoon schematic depicting a vaccine composition based on a linear amphiphilic carriers of formula S-B-[U]-H-D admixed with a peptide antigen conjugate of formula A-[E2]-[U]-H-D.

FIG. 5 shows the effect that varying solubilizing block functional group (i) composition, (ii) number and (iii) net charge have on the hydrodynamic behavior of vaccines comprising a peptide antigen conjugate of formula A-U-H-D admixed with an amphiphile of formula S-B-U-H-D having either linear, dendron or brush architecture, wherein the molar ratio of peptide antigen conjugate to amphiphile are 1:1. The peptide antigen conjugate is Compound 198. Note that whereas amphiphiles with linear architecture require net charge greater than or equal to +4 or less than or equal to −4 to induce stable nanoparticle micelles, amphiphiles with dendron architecture having net neutral charged formed nanoparticle micelles with more uniform size and consistency than amphiphiles with brush architecture. Particle size was assessed by dynamic light scattering and the number mean particle size in diameter (d, nm) is reported.

FIG. 6 shows T cell responses induced in mice (n=3/group) following vaccination with different cancer vaccine compositions or controls. Mice were vaccinated with either vehicle control (first column), nanoparticles comprising a peptide antigen conjugate of formula S-E1-A-E2-U-H-D (i.e., compound 249, second column) or mosaic nanoparticles comprising peptide antigen conjugates of formula A-U-H-D and A-U-E2-H-D (compounds 202 and 225, respectively) and an amphiphile of formula S-B-U-H-D with either net positive charge and linear architecture (compound 245, third column), net negative charge and linear architecture (compound 246, fourth column) or net neutral charge and dendron architecture (compound 162, fifth column). (A) CD8 and (B) CD4 T cell responses were determined from whole blood at day 13 after immunization using intracellular cytokine staining (ICS). Data are reported as the geometric mean with 95% confidence interval. Note the peptide antigen conjugates, compounds 249 and 202 comprise a neoantigen, Adpgk, derived from a melanoma cell line, MC-38, and the peptide antigen conjugate, compound 225 comprises a universal CD4 T cell epitope referred to as PADRE.

FIG. 7A shows the study design and T cell responses in mice (n=5/group) following immunization with different cancer vaccine compositions comprising neoantigens (Reps1, Adpgk, Cpne1 and Irgq) used alone or combined with a vaccinia virus as the source of adjuvant. (A) The treatment groups are listed in the table, which summarizes the compositions used for the prime (first immunization at day 0) and boost (second immunization at day 14) immunizations. Groups 1 to 11 were treated with vaccines comprising nanoparticles further comprising peptide antigen conjugates given alone or co-administered with a vaccinia virus (‘virus’). Except for group 2, the vaccinia virus did not encode the neoantigens.

FIG. 7B-C show antigen-specific CD8 T cell responses determined from whole blood at (B) day 7 and (C) day 28 after immunization using intracellular cytokine staining (ICS). Data are reported as the geometric mean with 95% confidence interval. Note: group 11, which were treated with mosaic nanoparticles comprising an amphiphile of formula S-B-U-H with neutral charge and dendron architecture and peptide antigen conjugates of formula A-U-H induced the highest magnitude CD8 T cell responses after a single immunization.

FIG. 8 shows cartoon schematics of mosaic nanoparticles formed by admixing amphiphilic carriers of formula S-B-[U]-H-D having brush architecture with peptide antigen conjugates of formula S-A-E2-[U]-H-D with either short or long extension (E2) sequences, which is meant to control the extent to which the antigen (A) is solvent exposed.

FIG. 9 shows the effect that varying the peptide antigen conjugate composition and amphiphilic carrier net charge, spacer length and architecture has on the hydrodynamic behavior of mosaic nanoparticle-based vaccines for inducing antibody responses. Each data point on the graph represents the particle size determined for a single mosaic nanoparticle composition comprising an amphiphile of formula S-B-U-H-D and a peptide antigen conjugate at a 1:1 molar ratio. Mosaic nanoparticles were formulated by combining the amphiphile and peptide antigen conjugate at a 1:1 molar ratio in DMSO, and then diluting the DMSO solution with PBS buffer, pH 7.4 to a final concentration of 0.05 mM. Particle size was assessed by dynamic light scattering and the number mean particle size in diameter (d, nm) is reported. Note that the amphiphiles with linear architecture led to uniformly sized nanoparticle micelles whereas the particle size variability is higher for the mosaic nanoparticles comprising amphiphiles with brush architecture.

FIG. 10 shows the hydrodynamic behavior of different compositions of vaccines against SARS-CoV-2. Peptide antigens conjugates of formula H-D-U-E1-A (compounds 207, 212 and 216) or HD-U-E1-A-S having a solubilizing block (compounds 208, 213 and 217) were admixed with a peptide antigen conjugate of formula A-U-H-D comprising a universal CD4 T cell epitope (compound 225) and an amphiphile of formula S-B-U-H-D at a 1:1 molar ratio of total peptide antigen conjugate to total amphiphile. Mosaic nanoparticles were formulated by combining the amphiphile and peptide antigen conjugates at a 1:1 molar ratio in DMSO, and then diluting the DMSO solution with PBS buffer, pH 7.4 to a final concentration of 0.05 mM. Particle size was assessed by dynamic light scattering and the number mean particle size in diameter (d, nm) is reported. Note that the amphiphiles with linear or dendron architecture led to uniformly sized nanoparticle micelles whereas the particle size variability is higher for the mosaic nanoparticles comprising amphiphiles with brush architecture.

FIG. 11 shows anti-SARS-CoV-2 antibody titers induced with different vaccine compositions. C57BL/6 (n=3/group) were immunized at days 0 and 14 with the indicated vaccine composition and antibody titers against three unique SARS-CoV-2 epitopes were assessed from whole blood at day 28 by sandwich ELISA. The midpoint antibody titers elicited against each of the epitopes is shown.

FIG. 12 shows anti-SARS-CoV-2 antibody titers induced with different vaccine compositions. C57BL/6 (n=3/group) were immunized at days 0 and 14 with the indicated vaccine composition and antibody titers against a single SARS-CoV-2 epitope was assessed from whole blood at day 28 by sandwich ELISA. The midpoint antibody titers elicited against the epitope is shown.

FIG. 13 shows the chemical structure of (a) methionine (M or Met) and the amino acid norleucine (n or nLeu) used as substitute for methionine, and (b) cysteine alpha-amino butyric acid (B or aBut) used as a substitute for cysteine.

FIG. 14A-E shows that peptide antigen conjugates comprising a peptide antigen further comprising a T cell epitope with the naturally occurring (or “native”) methionine residue replaced with norleucine elicit de novo T cell responses that recognize both the native antigen and the non-natural antigen (i.e. the antigen with methionine substituted with norleucine). (a) Sequences of the tumor self-antigen (native Trp1) and norleucine-substituted Trp1 (norTrp1) contained within compounds 257 and 258. (b) C57BL/6 mice (n=10-15 per group) were vaccinated subcutaneously (SC) on day 0 and 14 (with either 8 nmol of 257 or 258), bled on day 7 and 21, challenged subcutaneously with 10⁵ cells of B16-F10 on day 28, and treated with anti-PD-L1 on day 35 and 42. (c) CD8 T cell responses on day 21 as measured by intracellular cytokine staining for IFN-γ following peptide stimulation in vitro for 6 hours. Each sample was split and stimulated with either native Trp1 min or norTrp1 min at 2.5 μg/mL. Histograms show concatenated samples of all acquired events for each condition (left), and individual samples quantified with lines connecting paired samples (right). P-values are by one-way ANOVA (Kruskal-Wallis with Dunn's correction) comparing responses to Trp1 min across groups. (d) CD8 T cell responses on day 21 as measured by tetramer staining with TAPDNLGYM:H-2D^{b}-PE. Histograms show concatenated samples of all acquired events for each vaccine group (left), and bar graph shows quantification of each individual sample (right). P-values are by one-way ANOVA (Kruskal-Wallis with Dunn's correction) comparing responses across all groups. Data are mean±standard deviation. (e) Tumor growth curves (left) and survival (right) following tumor challenge. P-values are for survival curves and are by log-rank test with correction for multiple comparisons.

FIG. 15A-E shows that peptide antigen conjugates comprising a peptide antigen further comprising a T cell epitope with three naturally occurring (or “native”) methionine residues all replaced with norleucine elicit de novo T cell responses that recognize both the native antigen and the non-natural antigen (i.e. the antigen with each methionine substituted with norleucine). (a) Sequences of the tumor neoantigen (native Adpgk) and norleucine-substituted Adpgk (norAdpgk) contained within compounds 259 and 260. (b) C57BL/6 mice (n=10-15 per group) were vaccinated subcutaneously (SC) on day 0 and 14 (with either 8 nmol of 295 or 260). (b) C57BL/6 mice (n=10-15 per group) were vaccinated subcutaneously (SC) on day 0 and 14, bled on day 7 and 21, challenged subcutaneously with 10⁵ cells of B16-BFP-Adpgk on day 28, and treated with anti-PD-L1 on day 35 and 42. (c) CD8 T cell responses on day 21 as measured by intracellular cytokine staining for IFN-γ following peptide stimulation in vitro for 6 hours. Each sample was split and stimulated with either native Adpgk min or norAdpgk min at 2.5 μg/mL. Histograms show concatenated samples of all acquired events for each condition (left), and individual samples quantified with lines connecting paired samples (right). P-values are by one-way ANOVA (Kruskal-Wallis with Dunn's correction) comparing responses to Adpgk min across groups. (d) CD8 T cell responses on day 21 as measured by tetramer staining with ASMTNMELM:H-2D^{b}-PE. Histograms show concatenated samples of all acquired events for each vaccine group (left), and bar graph shows quantification of each individual sample (right). P-values are by one-way ANOVA (Kruskal-Wallis with Dunn's correction) comparing responses across all groups. Data are mean±standard deviation. (e) Tumor growth curves (left) and survival (right) following tumor challenge. P-values are for survival curves and are by log-rank test with correction for multiple comparisons.

FIG. 16A-F shows that peptide antigen conjugates comprising a peptide antigen further comprising a T cell epitope with three naturally occurring (or “native”) methionine residues all replaced with norleucine, but not norvaline or leucine, elicit de novo T cell responses that recognize both the native antigen and the non-natural antigen (i.e. the antigen with each methionine substituted with norleucine).
(a) Chemical structures of methionine, norleucine, norvaline and leucine. (b) Sequences of the tumor neoantigen (native Adpgk), norleucine-substituted Adpgk (norAdpgk or norleucine Adpgk), norvaline-substituted Adpgk (norvaline Adpk) and leucine-substituted Adpgk (leucine Adpgk) contained within compounds 259, 260, 261 and 262, respectively. (c) Reverse phase HPLC traces compounds 259, 260, 261 and 262; y-axis shows absorbance at 320 nm in arbitrary absorbance units (mAU). (d) C57BL/6 mice (n=5 per group) were vaccinated subcutaneously (SC) on day 0 and 14 with 8 nmol or either 259, 260 261 or 261 and bled on day 7 and 21 (e) CD8 T cell responses on day 7 as measured by tetramer staining with ASMTNMELM:H-2D^{b}-PE (left) or by intracellular cytokine staining for IFN-γ following peptide stimulation in vitro for 6 hours (right). Each sample was stimulated with Adpgk min at 2.5 μg/mL. P-values are by one-way ANOVA (Kruskal-Wallis with Dunn's correction). Data are mean±standard error of the mean. (f) CD8 T cell responses on day 21, measured as in e.

FIG. 17A-D shows that peptide antigen conjugates comprising a peptide antigen further comprising a T cell epitope with the naturally occurring (or “native”) cysteine residue replaced with alpha aminobutyric acid (B or aBut) elicits de novo T cell responses that recognize both the native antigen and the non-natural antigen (i.e. the antigen with cysteine replaced with aBut). (a) Sequences of the viral antigen (native gp33) and aBut-substituted gp33 (aBut gp33) contained within compounds 263 and 264. (b) C57BL/6 mice (n=9 per group) were vaccinated subcutaneously (SC) on day 0 and 14 with 8 nmol of compound 263 or 264 and bled on day 7 and 21. (c) CD8 T cell responses on day 21 as measured by intracellular cytokine staining for IFN-γ following peptide stimulation in vitro for 6 hours. Each sample was stimulated with gp33 min at 2.5 μg/mL. P-values are by one-way ANOVA (Kruskal-Wallis with Dunn's correction) comparing responses to gp33 min across groups. (d) Splenocytes were harvested (n=4 mice per group; each symbol represents a different mouse) and stimulated with gp33 min at the indicated concentrations for 6 hours and assessed for IFN-γ by intracellular cytokine staining. The y-axis is the % of the IFN-γ response at a given concentration as a percent of the response at 10,000 nM. The line is the non-linear sigmoidal regression (left). The interpolated EC₅₀ is graphed for each mouse (right). P-value is by Mann-Whitney.

FIG. 18A-D shows that peptide antigen conjugates comprising a peptide antigen further comprising a T cell epitope with the naturally occurring (or “native”) cysteine residue replaced with alpha aminobutyric acid (B or aBut) elicits de novo T cell responses that recognize both the native antigen and the non-natural antigen (i.e. the antigen with cysteine replaced with aBut). (a) Sequences of the tumor neoantigen (native m27) and aBut-substituted m27 (aBut m27) contained within compounds 266 and 267. (b) C57BL/6 mice (n=10 per group) were vaccinated subcutaneously (SC) on day 0 and 14 with 8 nmol of compound 266 or 267 and bled on day 7 and 21. (c) CD8 T cell responses on day 21 as measured by intracellular cytokine staining for IFN-γ following peptide stimulation in vitro for 6 hours. Each sample was stimulated with M27 min or aBut M27 min at 2.5 μg/mL. P-values are by one-way ANOVA (Kruskal-Wallis with Dunn's correction) comparing responses to M27 min across groups. (d) Memory phenotype on day 21 of IFN-γ+ cells following stimulation with M27 min. Data are mean±standard deviation.

FIG. 19A-E shows that peptide antigen conjugates comprising a peptide antigen further comprising a T cell epitope with the naturally occurring (or “native”) cysteine residue replaced with serine (S or Ser) elicit significantly lower magnitude T cell responses than those comprising the native sequence. (a) Chemical structures of cysteine and serine. (b) Sequences of the viral antigen (native gp33) and ser-substituted gp33 (ser gp33) contained within compounds 263 and 265. (c) C57BL/6 mice (n=5 per group) were vaccinated subcutaneously (SC) on day 0 and 14 with 8 nmol of compound 263 or 265 and bled on day 7 and 21. (d-e) CD8 T cell responses on day 7 (d) and day 21 (e) as measured by intracellular cytokine staining for IFN-γ following peptide stimulation in vitro for 6 hours. Each sample was split and stimulated with either gp33 min or ser gp33 min at 2.5 μg/mL. Lines connect paired samples (right). P-values are by one-way ANOVA (Kruskal-Wallis with Dunn's correction) comparing responses to gp33 min across groups. Note: the naive group was only stimulated with the gp33 min.

FIG. 20A-C shows that peptide antigen conjugates comprising a peptide antigen with three naturally occurring (or “native”) cysteine residues all replaced with aBut elicit de novo T cell responses that recognize both the native antigen and the non-natural antigen (i.e., the antigen with each cysteine substituted with aBut). (a) Sequences of the viral antigen (native E7) and aBut-substituted E7 (aBut E7) contained within compounds 268 and 269. (b) C57BL/6 mice (n=5 per group) were vaccinated subcutaneously (SC) on day 0 and 14 with 8 nmol of compound 268 or 269 and bled on day 21. (c) CD8 T cell responses on day 21 are shown.

FIG. 21A-21B shows the pH-responsiveness of exemplary amphiphiles with S blocks comprising carboxylics acid solubilizing groups. (a) Structures of the amphiphiles: Compounds 160, 270 and 271 comprise a PEG-based dendron amplifier with R²⁵ selected from beta-alanine, glycine and —OH, respectively. Compound 272 comprises a peptide-based dendron amplifier with R²⁶ selected from glutaric acid and Compounds 272 and 273 both comprise a peptide-based dendron amplifier with R²⁶ selected from succinic acid, but for compound 273 the spacer, B, is absent. U comprises a triazole and the H block is Ahx-2B3W2. (b) Absorbance at 490 nm (turbidity) was assessed for reach of the different amphiphiles at 0.1 mM in PBS buffer at the specified pH. Absorbance values greater than about 0.04 in this study indicate that aggregation is occurring.

FIG. 22A-22B shows the impact that peptide antigen conjugate net charge, presence of amphiphile and amphiphile spacer (B) length have on zeta potential, hemolysis and particle size stability for different vaccine compositions. The results show that (a) decreasing net charge and the presence of a neutral amphiphile lead to decreased RBC lysis; and (b) increasing net charge leads to improved particle size stability.

FIG. 23 shows the impact that peptide antigen conjugate net charge has on particle size stability for vaccine compositions with varying amphiphile composition. The results show that increasing peptide antigen conjugate net charge leads to improved particle size stability independent of the amphiphile composition.

FIG. 24 shows the impact that peptide antigen conjugate net charge and presence of an amphiphile have on particle size stability for vaccine compositions undergoing multiple freeze-thaw cycles. The results show that increasing peptide antigen conjugate net charge leads to improved particle size stability and formulations comprising amphiphiles with PEG24 (or PEG36, data not shown) spacers have greater particle size stability as compared to those with PEG48 spacers, which exhibit an increased propensity for aggregation (as indicated by absorbance (‘turbidity’) greater than 0.05 in this study).

FIG. 25 shows the impact of different inhibitors and/or immunostimulants on the proportion of T cells expressing either Tbet or Foxp3.

FIG. 26A-B shows the impact of Torin-1, an ATP-competitive mTOR inhibitor, on the magnitude of Tregs induced by tolerance vaccines with different immunostimulants. (a) The results show that Torin-1 (or ‘Torin’) combined with the TLR-7/8a, 2BXy, leads to the highest magnitude Treg responses, (b) which are 2-fold higher than those induced with Torin-1 alone, and 8-fold higher than the vaccine without Torin-1. Note: the concentration of peptide antigen conjugate is 500 nM; the molar ratio of total peptide antigen conjugate to Treg promoting immunomodulator is 1:1 at 500 nM of inhibitor, 1:0.25 at 125 nM of inhibitor and 1:0.1 at 50 nM of inhibitor. The results show that vaccines for inducing tolerance comprising an exemplary mTORC1/2 inhibitor, Torin1, leads to a higher proportion of antigen specific CD4 T cells expressing FOXP3 as compared with vaccines comprising an exemplary RORγt inhibitor (SR1555), an exemplary mTORC1 inhibitor, rapamycin and an exemplary AHR agonist, ITE.

FIG. 27A-27B shows the impact that the composition of E2 has on antibody responses generated against a peptide antigen (A) further comprising a minimal immunogen. (a) Shows the vaccination and sampling schedule; (b) shows the magnitude of antibody responses (total IgG) against SARS-CoV2 spike protein assessed from the serum of immunized mice 28 days after the first vaccination.

FIG. 28A-28B shows the impact the impact that E1 and/or E2 have on T cell activation in vitro. (a, b) In vitro antigen presentation was assessed by incubating different peptide antigen fragments of formula [S]-[E1]-A-[E2]-[U1] or [U1]-[E1]-A-[E2]-[S], wherein A is RAHYNIVTF or AQLANDVVL for (a) and (b), respectively, over a range of concentrations (0.001 to 10,000 nmol) with CD8 T cells and measuring the IFN-γ response by intracellular cytokine staining. EC50 for each peptide antigen fragment was determined by fitting a curve to the data expressed as % maximal response versus concentration and calculating the concentration at half-maximal response.

FIG. 29A-29B shows how vaccine regimen (homologous or heterologous prime-boost) and route of the boost impacts T cell responses. (a) Shows the vaccination and sampling schedule; note: peptide antigen conjugates were dosed at 32 nmol/mouse in 1001 PBS and ChAdOx was dosed at 1e8 infectious units (IU) in 100 ul PBS. (b) Shows the magnitude of antigen-specific T cells (% tetramer+ of total CD8+ T cells) induced by the different vaccine regimens at days 7 and 28 as assessed by flow cytometry.

FIG. 30A-30B shows how route and vaccine regimen (homologous or heterologous prime-boost) impacts T cell responses. (a) Shows the vaccination and sampling schedule; note: peptide antigen conjugates were dosed at 32 nmol/mouse in 100 μl PBS by the IV route or at 8 nmol/mouse in 100 μl PBS by the IM route; and, ChAdOx was dosed at 1e8 infectious units (IU) in 100 ul PBS. (b) Shows the magnitude of antigen-specific T cells (% tetramer+ of total CD8+ T cells) induced by the different vaccine regimens at days 7 and 28 as assessed by flow cytometry.

FIG. 31A-31B shows how peptide antigen conjugate net charge and/or presence of an amphiphile impacts T cell responses. (a) Shows the vaccination and sampling schedule; note: peptide antigen conjugates were dosed at 32 nmol/mouse in 100 μl PBS and ChAdOx was dosed at 1e8 infectious units (IU) in 100 ul PBS. (b) Shows the magnitude of antigen-specific T cells (% tetramer+ of total CD8+ T cells) induced by the different vaccine regimens at days 7 and 28 as assessed by flow cytometry.

FIG. 32A-32B shows that ChAdOx can be used as a biological adjuvant. (a) Shows the vaccination and sampling schedule; note: peptide antigen conjugates were dosed at 32 nmol/mouse in 100 μl PBS for the prime but 32 nmol/mouse in 100 μl PBS for the boost; and ChAdOx was dosed at 1e8 infectious units (IU) in 100 ul PBS. (b) Shows the magnitude of antigen-specific T cells (% tetramer+ of total CD8+ T cells) induced by the different vaccine regimens at days 7 and 28 as assessed by flow cytometry.

### DEFINITIONS

Details of terms and methods are given below to provide greater clarity concerning compounds, compositions, methods and the use(s) thereof for the purpose of guiding those of ordinary skill in the art in the practice of the present disclosure. The terminology in this disclosure is understood to be useful for the purpose of providing a better description of particular embodiments and should not be considered limiting.

About: In the context of the present disclosure, “about” when referring to a measurable value such as an amount, a temporal duration, and the like, is meant to encompass variations of ±20%, ±10%, ±5%, ±1%, or ±0.1% from the specified value, as such variations are appropriate to perform the disclosed methods. For example, “about 10” refers to 9.5 to 10.5. A ratio of “about 5:1” refers to a ratio from 4.75:1 to 5.25:1.

Administration: To provide or give to a subject an agent, for example, an immunogenic composition comprising amphiphilic block copolymers and drug(s) as described herein, by any effective route. Exemplary routes of administration include, but are not limited to, oral, injection (such as subcutaneous, intramuscular, intradermal, intraperitoneal, and intravenous), transdermal, topical, intranasal, vaginal, and inhalation routes.

“Administration of” and “administering a” compound should be understood to mean providing a compound, a prodrug of a compound, or a pharmaceutical composition as described herein. The compound or composition can be administered by another person to the subject or it can be self-administered by the subject.

Antigen: Any molecule that contains an epitope that binds to a T cell or B cell receptor and can stimulate an immune response, in particular, a B cell response and/or a T cell response in a subject. The epitopes may comprise peptides, glycopeptides, lipids or any suitable molecules that contain an epitope that can interact with components of specific B cell or T cell receptors. Such interactions may generate a response by the immune cell. “Epitope” refers to the region of a peptide antigen to which B and/or T cell proteins, i.e., B-cell receptors and T-cell receptors, interact. Antigens used in embodiments of the present disclosure may be selected from pathogens, cancerous cells, autoantigens, alloantigens or allergens. Many such antigens may be used according to embodiments of the inventions of the present disclosure and are discussed in greater detail throughout this specification.

Antigen-presenting cell (APC): Any cell that presents antigen bound to MHC class I or class II molecules to T cells, including but not limited to monocytes, macrophages, dendritic cells, B cells, T cells and Langerhans cells.

Amphiphilic: The term “amphiphilic” is used herein to mean a substance containing both hydrophilic or polar and hydrophobic groups.

CD4: Cluster of differentiation 4, a surface glycoprotein that interacts with MHC Class II molecules present on the surface of other cells. A subset of T cells express CD4 and these cells are commonly referred to as helper T cells or CD4 T cells.

CD8: Cluster of differentiation 8, a surface glycoprotein that interacts with MHC Class I molecules present on the surface of other cells. A subset of T cells express CD8 and these cells are commonly referred to as cytotoxic T cells (CTLs), killer T cells or CD8 T cells.

Charge: A physical property of matter that affects its interactions with other atoms and molecules, including solutes and solvents. Charged matter experiences electrostatic force from other types of charged matter as well as molecules that do not hold a full integer value of charge, such as polar molecules. Two charged molecules of like charge repel each other, whereas two charged molecules of different charge attract each other. Charge is often described in positive or negative integer units. The charge of a molecule can be readily estimated based on the molecule's Lewis structure and accepted methods known to those skilled in the art. Charge may result from inductive effects, e.g., atoms bonded together with differences in electron affinity may result in a polar covalent bond resulting in a partially negatively charged atom and a partially positively charged atom. For example, nitrogen bonded to hydrogen results in partial negative charge on nitrogen and a partial positive charge on the hydrogen atom. Alternatively, an atom in a molecule may be considered to have a full integer value of charge when the number of electrons assigned to that atom is less than or equal to the atomic number of the atom. The charge of the molecule is determined by summing the charge of each atom comprising the molecule. Those skilled in the art are familiar with the process of estimating charge of a molecule by summing the formal charge of each atom in a molecule. “Charged functional groups refer to functional groups that may be permanently charged or have charge depending on the pH. Charged functional groups may be partial or full integer values of charge, which may be positive or negative, are referred to as positively charged functional groups or negatively charged functional groups, respectively. The portion of a molecule that comprises one or more charged functional groups, which may be positive or negative, is referred to as a “charged group,” e.g., positively charged group or negatively charged group. Charged groups may comprise positive functional groups, negative functional groups or both positive and negative functional groups. The net charge of the charged group may be positive, negative or neutral. Charged monomers refer to monomers that comprise charged groups. Charged amino acids are a type of charged monomer. Note: the net charge of a particle comprising amphiphiles and/or peptide antigen conjugates further comprising charged groups, e.g., charged monomers, such as charged amino acids, can be estimated by summing the charge of each functional group within the amphiphiles and/or peptide antigen conjugates.

Chemotherapeutic: is a type of drug molecule (D) defined broadly as any pharmaceutically active molecule useful in the treatment of cancer and includes growth inhibitory agents or cytotoxic agents, including alkylating agents, anti-metabolites, anti-microtubule inhibitors, topoisomerase inhibitors, receptor tyrosine kinase inhibitors, angiogenesis inhibitors and the like. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylmelamines including altretamine, triethylenemelamine, triethylenephosphoramide, triethylenethiophosphoramide and trimethylolomelamine; nitrogen mustards such as chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-FU; folic acid analogues such as denopterin, methotrexate, pteropterin, trimetrexate; purine analogues such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; members of taxoid or taxane family, such as paclitaxel (TAXOL®), docetaxel (TAXOTERE®) and analogues thereof; chlorambucil; gemcitabine; 6-thioguanine; mercaptopurine; methotrexate; platinum analogues such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; inhibitors of receptor tyrosine kinases and/or angiogenesis, including sorafenib (NEXAVAR®), sunitinib (SUTENT®), pazopanib (VOTRIENT™), toceranib (PALLADIA™) vandetanib (ZACTIMA™), cediranib (RECENTIN®), regorafenib (BAY 73-4506), axitinib (AG013736), lestaurtinib (CEP-701), erlotinib (TARCEVA®), gefitinib (IRESSA™), BIBW 2992 (TOVOK™), lapatinib (TYKERB®), neratinib (HKI-272), and the like, and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (FARESTON®); and anti-androgens such as flutamide, nilutamide, bicalutamide, leuprolide, and goserelin; and pharmaceutically acceptable salts, acids or derivatives of any of the above. Other conventional cytotoxic chemical compounds as those disclosed in Wiemann et al., 1985, in Medical Oncology (Calabresi et al, eds.), Chapter 10, McMillan Publishing, are also suitable chemotherapeutic agents.

Chemotherapeutics (also referred to as chemotherapeutic agents) are pharmaceutically active compounds and may therefore be referred to herein generally as drugs or drug molecules, or “D” in formulae. For clarity, the terms chemotherapeutic(s) and chemotherapeutic agent(s) are used herein to describe any synthetic or naturally occurring molecules useful for cancer treatment, though, certain classes of drug molecules may alternatively be described by their mechanism of action, e.g., angiogenesis inhibitors are chemotherapeutics that inhibit angiogenesis. While certain immunomodulators, e.g., immunostimulants, may be useful for cancer treatment, immunomodulators, inclusive of immunostimulants and immunosuppressants are not referred to as chemotherapeutics in this specification.

Click chemistry reaction: A bio-orthogonal reaction that joins two compounds together under mild conditions in a high yield reaction that generates minimal, biocompatible and/or inoffensive byproducts. An exemplary click chemistry reaction used in the present disclosure is the reaction of an azide group with an alkyne to form a triazole linker through strain-promoted [3+2]azide-alkyne cyclo-addition.

Copolymer: A polymer derived from two (or more) different monomers, as opposed to a homopolymer where only one monomer is used. Since a copolymer includes at least two types of constituent units (also structural units), copolymers may be classified based on how these units are arranged along the chain. A copolymer may be a statistical (or random) copolymer wherein the two or monomer units are distributed randomly; the copolymer may be an alternating copolymer wherein the two or more monomer units are distributed in an alternating sequence; or, e.g., the copolymer, e.g., a poly(amino acid) may be produced by solid-phase peptide synthesis (SPPS) and have a specific order of monomer units. The term “block copolymer” refers generically to a polymer composed of two or more contiguous blocks of different constituent monomers or comonomers (if a block comprises two or more different monomers). Block copolymer may be used herein to refer to a copolymer that comprises two or more homopolymer subunits, two or more copolymer subunits or one or more homopolymer subunits and one or more copolymer subunits, wherein the subunits may be linked directly by covalent bonds or the subunits may be linked indirectly via an intermediate non-repeating subunit, such as a junction block or linker. Blocks may be based on linear and/or brush architectures. Block copolymers with two or three distinct blocks are referred to herein as “diblock copolymers” and “triblock copolymers,” respectively. Copolymers may be referred to generically as polymers, e.g., a statistical copolymer may be referred to as a polymer or copolymer. Similarly, a block copolymer may be referred to generically as a polymer. While a copolymer used in herein means a polymer comprising two or more types of monomers, terpolymer is a copolymer with three monomer units.

Critical micelle concentration (CMC): Refers to the concentration of a material above which micelles spontaneously form to satisfy thermodynamic equilibrium.

Drug: refers to any pharmaceutically active molecule—including, without limitation, proteins, peptides, sugars, saccharides, nucleosides, inorganic compounds, lipids, nucleic acids, small synthetic chemical compounds, macrocycles, etc.—that has a physiological effect when ingested or otherwise introduced into the body. Pharmaceutically active compounds can be selected from a variety of known classes of compounds, including, for example, analgesics, anesthetics, anti-inflammatory agents, anthelmintics, anti-arrhythmic agents, antiasthma agents, antibiotics (including penicillins), anticancer agents, anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, such as therapeutic antibodies and antibody fragments, MHC-peptide complexes, cytokines and growth factors, glycoproteins, peptides and polypeptides, parasympathomimetics, parathyroid calcitonin, biphosphonates, prostaglandins, radio-pharmaceuticals, hormones, sex hormones (including steroids), anti-allergic agents, stimulants and anoretics, steroids, sympathomimetics, thyroid agents, vaccines, vasodilators, and xanthines. Drugs may also be referred to as pharmaceutically active agents, pharmaceutically active substances or biologically active compounds or bioactive molecules. Any drug molecules in the formulae described herein are abbreviated “D.”

Drug delivery: A method or process of administering a pharmaceutical compound to achieve a therapeutic effect in humans or animals.

Effective amount: The amount of a compound, material, or composition effective to achieve a particular biological result such as, but not limited to, biological results disclosed, described, or exemplified herein. Such results may include, but are not limited to, the effective reduction of symptoms associated with any of the disease states mentioned herein, as determined by any means suitable in the art.

Graft copolymer: A polymer having a main polymer chain (e.g., polymer A) with one or more sidechains of a second polymer (e.g., polymer B). The first polymer A is linked through its monomers and sidechains to the second polymer B, which is bonded to individual monomers of polymer A thereby branching off from the chain of polymer A. A first polymer linked through an end group to a second polymer may be described as a block polymer (e.g., A-B type di-block) or an end-grafted polymer.

Hydropathy index/GRAVY value: Is a number representing the hydrophobic or hydrophilic characteristics of an amino acid or sequence of amino acids. There are a variety of scales that can be used to describe the relative hydrophobic and hydrophilic characteristics of amino acids comprising peptides. In the present disclosure, the Hydropathy scale of Kyte and Doolittle (Kyte J, Doolittle R F, J. Mol. Biol 157: 105-32, 1983) is used to calculate the grand average of hydropathy (GRAVY) value, sometimes referred to as the GRAVY score. The GRAVY value of a peptide is the sum of the Hydropathy values of all amino acids comprising the peptide divided by the length (i.e., number of amino acids) of the peptide. The GRAVY value is a relative value. The larger the GRAVY value, the more hydrophobic a peptide sequence is considered, whereas the lower the GRAVY value, the more hydrophilic a peptide sequence is considered.

Hydrophilic: Refers to the tendency of a material to disperse freely or be solubilized in aqueous solutions (sometimes referred to as aqueous media). A material is considered hydrophilic if it prefers interacting with other hydrophilic material and avoids interacting with hydrophobic material. In some cases, hydrophilicity may be used as a relative term, e.g., the same molecule could be described as hydrophilic or not depending on what it is being compared to. Hydrophilic molecules are often polar and/or charged and have good water solubility, e.g., are soluble at concentrations of at least 1.0 mg/mL or more. Hydrophilic group refers to the portion of a molecule that is polar and/or charged and has good water solubility.

Hydrophobic: Refers to the tendency of a material to avoid contact with water. A material is considered hydrophobic if it prefers interacting with other hydrophobic material and avoids interacting with hydrophilic material. Hydrophobicity is a relative term; the same molecule could be described as hydrophobic or not depending on what it is being compared to. Hydrophobic molecules are often non-polar and non-charged and have poor water solubility, e.g., are insoluble in water, or are soluble in water only at concentrations of 1 mg/mL or less, typically 0.1 mg/mL or less or more preferably 0.01 mg/mL or less. Hydrophobic monomers are monomers, e.g., hydrophobic amino acids, that comprise hydrophobic groups and form polymers that are insoluble in water or insoluble in water at certain temperatures, pH and salt concentration. Hydrophobic group refers to a portion of a molecule that is hydrophobic. For example, a styrene monomer may be referred to as a hydrophobic monomer because poly(styrene) is a water insoluble polymer. Hydrophobic drugs refer to drug molecules that are insoluble or soluble only at concentrations of about 1.0 mg/mL or less in aqueous solutions at pH of about pH 7.4. Amphiphilic drugs are drug molecules that have the tendency to assemble into supramolecular structures, e.g., micelles, in aqueous solutions and/or have limited solubility in aqueous solutions at pH of about pH 7.4.

Immune response: A change in the activity of a cell of the immune system, such as a B cell, T cell, or monocyte, as a result of a stimulus, either directly or indirectly, such as through a cellular or cytokine intermediary. In certain embodiments, the response is specific for a particular antigen (an “antigen-specific response”). An immune response may comprise a T cell response, such as a CD4 T cell response or a CD8 T cell response. Such an immune response may result in the production of additional T cell progeny and/or in the movement of T cells. In other embodiments, the response is a B cell response, and results in the production of specific antibodies or the production of additional B cell progeny. In yet other embodiments, the response is an antigen-presenting cell response. An antigen may be used to stimulate an immune response leading to the activation of cytotoxic T cells that kills virally infected cells or cancerous cells. In other embodiments, an antigen may be used to induce tolerance or immune suppression. A tolerogenic response may result from the unresponsiveness of a T cell or B cell to an antigen. A suppressive immune response may result from the priming and/or activation of regulatory cells, such as regulatory T cells, or the trans-differentiation of effectors cells to regulatory cells that downregulate the immune response, i.e., dampen the immune response.

Immunogenic composition: A formulation of materials comprising an antigen and optionally an immunomodulator that induces a measurable immune response against the antigen. For examples, vaccines are a type of immunogenic composition.

Immunomodulators: refers to a type of drug that modulates the activity of cells of the immune system, which includes immunostimulants and immunosuppressants.

Immunostimulants: refers to any synthetic or naturally occurring drugs that promote pro-inflammatory and/or cytotoxic activity by immune cells. Exemplary immunostimulants include pattern recognition receptor (PRR) agonists, such as synthetic or naturally occurring agonists of Toll-like receptors (TLRs), stimulator of interferon gene agonists (STINGa), nucleotide-binding oligomerization domain-like receptor (NLR) agonists, retinoic acid-inducible gene-I-like receptors (RLR) agonists and certain C-type lectin receptor (CLR), as well as certain cytokines (e.g., certain interleukins), such as IL-2; certain chemokines or small molecules that bind chemokine receptors; certain antibodies, antibody fragments or synthetic peptides that activate immune cells, e.g., through binding to stimulatory receptors, e.g., anti-CD40, or, e.g., by blocking inhibitory receptors, e.g., anti-CTLA4, anti-PD1, etc. Various immunostimulants suitable for the practice of the present disclosure are described throughout the specification. For clarity, certain pharmaceutically active compounds that stimulate the immune system may be referred to as immunostimulants or more generally as drug molecules (abbreviated “D” in formulae).

Immunosuppressants: refers to any synthetic or naturally occurring drugs that suppress pro-inflammatory and/or cytotoxic activity by immune cells or the humoral immune system, e.g., antibodies and complement proteins. Immunosuppressants may mediate effects through one or more of the following mechanisms of action: by priming suppressor cells, e.g., regulatory T cells; killing, inhibiting or deactivating proinflammatory cells, cytotoxic cells and/or B cells; trans-differentiating proinflammatory and/or cytotoxic T cells to suppressor cells; and/or sequestering and/or limiting the mobility of proinflammatory cells, cytotoxic cells and/or B cells. Exemplary immunosuppressants include synthetic or naturally occurring agonists of the aryl hydrocarbon receptor (AHR); certain steroids, including glucocorticoids; certain histone deacetylase inhibitors (HDACS), such as inhibitors of HDAC9; retinoic acid receptor agonists; mammalian target of rapamycin (mTOR) inhibitors, such as rapamycin; certain cyclin dependent kinase (CDK) inhibitors; certain adenosine receptor agonists; agonists of PD1; and other molecules that suppress proinflammatory or cytotoxic activity by immune cells or antibodies. Various immunosuppressants suitable for the practice of the present disclosure are described throughout the specification and include Treg promoting immunomodulators. For clarity, immunosuppressants may be referred to more generally as drug molecules (abbreviated “D” in formulae).

In vivo delivery: Administration of a composition, such as a composition comprising amphiphilic block copolymers and drug(s), by topical, transdermal, suppository (rectal, vaginal), pessary (vaginal), intravenous, oral, subcutaneous, intraperitoneal, intrathecal, intramuscular, intracranial, inhalational, oral, or any other suitable route to a subject.

Linked or coupled: The terms “linked” and “coupled” mean joined together, either directly or indirectly. A first moiety may be covalently or noncovalently linked to a second moiety. In some embodiments, a first molecule is linked by a covalent bond to another molecule. In some embodiments, a first molecule is linked by electrostatic attraction to another molecule. In some embodiments, a first molecule is linked by dipole-dipole forces (for example, hydrogen bonding) to another molecule. In some embodiments, a first molecule is linked by van der Waals forces (also known as London forces) to another molecule. A first molecule may be linked by any and all combinations of such couplings to another molecule. The molecules may be linked indirectly, such as by using a linker (sometimes referred to as linker molecule). The molecules may be linked indirectly by interposition of a component that binds non-covalently to both molecules independently. The term “Linker,” sometimes abbreviated “X,” used in chemical formulae herein means any suitable linker molecule. Specific, preferred linkers may be indicated by other symbols, such as X1, X2, X3, X4, X5 and U. Various linkers are described throughout the specification.

A “bilayer membrane” or “bilayer(s)” is a self-assembled membrane of amphiphiles or super-amphiphiles in aqueous solutions.

Micelles: Spherical receptacles having a single monolayer defining a closed compartment. Generally, amphiphilic molecules spontaneously form micellar structures in polar solvents. In contrast to bilayers, e.g., liposomal bilayers, micelles are “sided” in that they project a hydrophilic, polar outer surface and display a hydrophobic interior surface.

Mol %: Refers to the percentage of a particular type of monomeric unit (or “monomer”) that is present in a polymer. For example, a polymer having 100 monomeric units of A and B with a density (or “mol %”) of monomer A equal to 10 mol % would have 10 monomeric units of A, and the remaining 90 monomeric units (or “monomers”) may be monomer B or another monomer unless otherwise specified.

Monomeric unit: The term “monomeric unit” or “monomer unit” is used herein to mean a unit of polymer molecule containing the same or similar number of atoms as one of the monomers. Monomeric units, as used in this specification, may be of a single type (homogeneous) or a variety of types (heterogeneous). For example, poly(amino acids) comprise amino acid monomeric units. Monomeric units may also be referred to as monomers or monomer units or the like.

Net charge: The sum of electrostatic charges carried by a molecule or, if specified, a portion or section of a molecule.

Particle: A nano- or micro-sized supramolecular structure composed of an assembly of molecules. For example, amphiphiles and peptide antigen conjugates of the present disclosure form particles in aqueous solution. In some embodiments, particle formation by the amphiphiles and/or peptide antigen conjugates is dependent on pH or temperature. In some embodiments, the nanoparticles composed of amphiphiles and/or peptide antigen conjugates have an average diameter between 5 nanometers (nm) to 500 nm. In some embodiments, the nanoparticles composed of amphiphiles and/or peptide antigen conjugates form micelles and have an average diameter between 5 nanometers (nm) to 50 nm, such as between 10 and 30 nm. In some embodiments, the nanoparticles composed of amphiphiles and/or peptide antigen conjugates may be larger than 100 nm.

Pattern recognition receptors (PRRs): Receptors expressed by various cell populations, particularly innate immune cells that bind to a diverse group of synthetic and naturally occurring molecules. There are several classes of PRRs. Non-limiting examples of PRRs include Toll-like receptors (TLRs), RIG-I-like receptors (RLRs), NOD-like receptors (NLRs), Stimulator of Interferon Genes receptor (STING), and C-type lectin receptors (CLRs). Agonists of such PRRs are referred to as immunostimulant drugs and can be used to enhance and/or modify an immune response to an antigen. For more information on pattern recognition receptors, see Wales et al., Biochem Soc Trans., 35:1501-1503, 2007.

Peptide or polypeptide: Two or more natural or non-natural amino acid residues that are joined together in a series through one or more amide bonds. The amino acid residues may contain post-translational modification(s) (e.g., glycosylation, citrullination, homocitrullination, oxidation and/or phosphorylation). Such modifications may mimic post-translational modifications that occur naturally in vivo or may be non-natural. Any one or more of the components of the amphiphiles and/or peptide antigen conjugates may comprise peptides.

Peptide Modifications: Peptides may be altered or otherwise synthesized with one or more of several modifications as set forth below. In addition, analogs (non-peptide organic molecules), derivatives (chemically functionalized peptide molecules obtained starting from a peptide) and variants (homologs) of these peptides can be utilized in the methods described herein. The peptides described herein comprise a sequence of amino acids, analogs, derivatives, and variants, which may be either L- and/or D-versions. Unless otherwise specified, any peptide sequences referenced herein comprise L amino acids, preferably exclusively L amino acids. Such peptides may contain peptides, analogs, derivatives, and variants that are naturally occurring and otherwise.

Peptides can be modified through any of a variety of chemical techniques to produce derivatives having similar activity as the unmodified peptides, and optionally having other desirable properties. For example, carboxylic acid groups of the peptide, whether at the carboxyl terminus or at a side chain, can be provided in the form of a salt of a pharmaceutically-acceptable cation or esterified to form a CC₁-CC₁₆ ester, wherein CC refers to a carbon chain (and thus, CC1 refers to a single carbon and CC16 refers to 16 carbons), or converted to an amide. Amino groups of the peptide, whether at the amino terminus or at a side chain, can be in the form of a pharmaceutically-acceptable acid addition salt, such as the HCl, HBr, acetic, trifluoroacetic, formic, benzoic, toluene sulfonic, maleic, tartaric and other organic salts, or can be modified or converted to an amide, e.g., by acetylation.

Peptides may be modified to contain substituent groups that contain a positive or negative charge or both. The positive and/or negative charge may be affected by the pH at which the peptide is present.

Hydroxyl groups of the peptide side chains may be converted to C₁-C₁₆ alkoxy or to a C₁-C₁₆ ester using well-recognized techniques, or the hydroxyl groups may be converted (e.g., sulfated or phosphorylated) to introduce negative charge. Phenyl and phenolic rings of the peptide side chains may be substituted with one or more halogen atoms, such as fluorine, chlorine, bromine or iodine, or with C₁-C₁₆ alkyl, C₁-C₁₆ alkoxy, carboxylic acids and esters thereof, or amides of such carboxylic acids. Methylene groups of the peptide side chains can be extended to homologous C₂-C₄ alkylenes. Thiols can be used to form disulfide bonds or thioethers, for example through reaction with a maleimide. Thiols may be protected with any of a number of well-recognized protecting groups, such as acetamide groups. Those skilled in the art will also recognize methods for introducing cyclic structures into the peptides of this invention to select and provide conformational constraints to the structure that result in enhanced stability. Reference may be made to Greene et al., “Greene's Protective Groups in Organic Synthesis” Fourth Edition, John Wiley & Sons, Inc. 2006 for details of additional modifications that can be made to functional groups.

An unexpected finding reported herein is that cysteine residues of naturally occurring peptide antigens can be replaced with alpha aminobutyric acid or serine, and methionine residues can be replaced with norleucine, to yield nonnatural peptide antigens that induce immune responses that are cross-reactive with the naturally occurring peptide antigens. Preferred methods for preparing and using peptide antigens with nonnatural sequences are described throughout the specification.

Pharmaceutically acceptable vehicles: The pharmaceutically acceptable vehicles (or carriers) useful in this disclosure include conventional carriers, excipients, and diluents. Remington's Pharmaceutical Sciences, by E. W. Martin, Mack Publishing Co., Easton, PA, 15th Edition (1975), describes compositions and formulations suitable for pharmaceutical delivery of one or more therapeutic compositions, such as one or more therapeutic cancer vaccines, and additional pharmaceutical agents.

Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection, or the like. The composition can also be present in a transdermal delivery system, e.g., a skin patch. The composition can also be present in a solution suitable for topical administration, such as an ointment or cream.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation of pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

Polar: A description of the properties of matter. Polar is a relative term and may describe a molecule or a portion of a molecule that has partial charge that arises from differences in electronegativity between atoms bonded together in a molecule, such as the bond between nitrogen and hydrogen. Polar molecules prefer interacting with other polar molecules and typically do not associate with non-polar molecules. In specific, non-limiting cases, a polar group may contain a hydroxyl group, or an amino group, or a carboxyl group, or a charged group. In specific, non-limiting cases, a polar group may prefer interacting with a polar solvent such as water. In specific, non-limiting cases, introduction of additional polar groups may increase the solubility of a portion of a molecule.

Polymer: A molecule containing repeating structural units (monomers). As described in greater detail throughout the disclosure, polymers may be used for any number of components of amphiphiles, peptide antigens conjugates and drug molecule conjugates and may be natural or synthetic. Various compositions of polymers useful for the practice of the invention are discussed in greater detail elsewhere. Note: polymer is used throughout the specification to broadly encompass molecules with as few as three or more monomers, which may sometimes be referred to as oligomers.

Polymerization: A chemical reaction, usually carried out with a catalyst, heat or light, in which monomers combine to form a chainlike, branched or cross-linked macromolecule (a polymer). The chains, branches or cross-linked macromolecules can be further modified by additional chemical synthesis using the appropriate substituent groups and chemical reactions. Polymerization commonly occurs by addition or condensation. Addition polymerization occurs when an initiator, usually a free radical, reacts with a double bond in the monomer. The free radical adds to one side of the double bond, producing a free electron on the other side. This free electron then reacts with another monomer, and the chain becomes self-propagating, thus adding one monomer unit at a time to the end of a growing chain. Condensation polymerization involves the reaction of two monomer units resulting in the splitting out of a water molecule. In other forms of polymerization, a monomer is added one at a time to a growing chain through the staged introduction of activated monomers, such as during solid phase peptide synthesis (SPPS).

Polymersome: Vesicle, which is assembled from synthetic multi-block polymers in aqueous solutions. Unlike liposomes, a polymersome does not include lipids or phospholipids as its majority component. Consequently, polymersomes can be thermally, mechanically, and chemically distinct and, in particular, more durable and resilient than the most stable of lipid vesicles. The polymersomes assemble during processes of lamellar swelling, e.g., by film or bulk rehydration or through an additional phoresis step, as described below, or by other known methods. Like liposomes, polymersomes form by “self-assembly,” a spontaneous, entropy-driven process of preparing a closed semi-permeable membrane.

Purified: A substance or composition that is relatively free of impurities or substances that adulterate or contaminate the substance or composition. The term purified is a relative term and does not require absolute purity. Substantial purification denotes purification from impurities. A substantially purified substance or composition is at typically at least 60%, 70%, 80%, 90%, 95%, 98%, or 99% pure.

Soluble: Capable of becoming molecularly or ionically dispersed in a solvent to form a homogeneous solution. When referring to an amphiphile, peptide antigen conjugate, drug molecule conjugate and/or drug molecule, soluble is understood to be a single molecule in solution that does not assemble into multimers or other supramolecular structures through hydrophobic or other non-covalent interactions. A soluble molecule is understood to be freely dispersed as single molecules in solution. Hydrophobic blocks (H) described herein are insoluble or soluble only to concentrations of about 0.1 mg/mL or less. Solubility can be determined by visual inspection, turbidity measurements or dynamic light scattering.

Subject and patient: These terms may be used interchangeably herein to refer to both human and non-human animals, including birds and non-human mammals, such as rodents (for example, mice and rats), non-human primates (for example, rhesus macaques), companion animals (for example domesticated dogs and cats), livestock (for example pigs, sheep, cows, llamas, and camels), as well as non-domesticated animals (for example big cats).

Targeting molecules: Are broadly defined as molecules that direct drug molecules to a specific tissue or cell population. Targeting molecules are defined by their intended use and therefore include structurally diverse molecules including without limitation antibodies, Fabs, peptides, aptamers, saccharides (e.g., saccharides that bind to lectin receptors and/or are recognized by cellular transporters), amino acids, neurotransmitters, etc. As targeting molecules are often selected from molecules that bind cellular receptors that can activate downstream signaling cascades and/or impact the activity of other linked molecules, targeting molecules are often classified as drug molecules (D) in the present disclosure. Additionally, targeting molecules can also have solubilizing effects, and may be considered either or both drug molecules (D) and/or solubilizing (SG) groups.

T Cell: A type of white blood cell that is part of the immune system and may participate in an immune response. T cells include, but are not limited to, CD4 T cells and CD8 T cells. A CD4 T cell displays the CD4 glycoprotein on its surface and these cells are often referred to as helper T cells. These cells often coordinate immune responses, including antibody responses and cytotoxic T cell responses, however, CD4 T cells (e.g., regulatory T cells) can also suppress immune responses or CD4 T cells may act as cytotoxic T cells. A CD8 T cell displays the CD8 glycoprotein on its surface and these cells are often referred to as cytotoxic or killer T cells, however, CD8 T cells can also suppress immune responses.

Treating, preventing, or ameliorating a disease: “Treating” refers to an intervention that reduces a sign or symptom or marker of a disease or pathological condition after it has begun to develop. For example, treating a disease may result in a reduction in tumor burden, meaning a decrease in the number or size of tumors and/or metastases, or treating a disease may result in immune tolerance that reduces systems associated with autoimmunity. “Preventing” a disease refers to inhibiting the full development of a disease. A disease may be prevented from developing at all. A disease may be prevented from developing in severity or extent or kind. “Ameliorating” refers to the reduction in the number or severity of signs or symptoms or marker of a disease, such as cancer.

Reducing a sign or symptom or marker of a disease or pathological condition related to a disease, refers to any observable beneficial effect of the treatment and/or any observable effect on a proximal, surrogate endpoint, for example, tumor volume, whether symptomatic or not. Reducing a sign or symptom associated with a tumor or viral infection can be evidenced, for example, by a delayed onset of clinical symptoms of the disease in a susceptible subject (such as a subject having a tumor which has not yet metastasized, or a subject that may be exposed to a viral infection), a reduction in severity of some or all clinical symptoms of the disease, a slower progression of the disease (for example by prolonging the life of a subject having a tumor or viral infection), a reduction in the number of relapses of the disease, an improvement in the overall health or well-being of the subject, or by other parameters well known in the art (e.g., that are specific to a particular tumor or viral infection). A “prophylactic” treatment is a treatment administered to a subject who does not exhibit signs of a disease or exhibits only early signs for the purpose of decreasing the risk or severity of developing pathology.

Tumor or cancer or neoplasm: An abnormal growth of cells, which can be benign or malignant, often but not always causing clinical symptoms. “Neoplastic” cell growth refers to cell growth that is not responsive to physiologic cues, such as growth and inhibitory factors.

A “tumor” is a collection of neoplastic cells. In most cases, tumor refers to a collection of neoplastic cells that forms a solid mass. Such tumors may be referred to as solid tumors. In some cases, neoplastic cells may not form a solid mass, such as the case with some leukemias. In such cases, the collection of neoplastic cells may be referred to as a liquid cancer.

Cancer refers to a malignant growth of neoplastic cells, being either solid or liquid. Features of a cancer that define it as malignant include metastasis, interference with the normal functioning of neighboring cells, release of cytokines or other secretory products at abnormal levels and suppression or aggravation of inflammatory or immunological response(s), invasion of surrounding or distant tissues or organs, such as lymph nodes, etc.

A tumor that does not present substantial adverse clinical symptoms and/or is slow growing is referred to as “benign.”

“Malignant” means causing, or likely to cause in the future, significant clinical symptoms. A tumor that invades the surrounding tissue and/or metastasizes and/or produces substantial clinical symptoms through production and secretion of chemical mediators having an effect on nearby or distant body systems is referred to as “malignant.”

“Metastatic disease” refers to cancer cells that have left the original tumor site and migrated to other parts of the body, for example via the bloodstream, via the lymphatic system, or via body cavities, such as the peritoneal cavity or thoracic cavity.

The amount of a tumor in an individual is the “tumor burden”. The tumor burden can be measured as the number, volume, or mass of the tumor, and is often assessed by physical examination, radiological imaging, or pathological examination.

An “established” or “existing” tumor is a tumor that exists at the time a therapy is initiated. Often, an established tumor can be discerned by diagnostic tests. In some embodiments, an established tumor can be palpated. In some embodiments, an established tumor is at least 500 mm³, such as at least 600 mm³, at least 700 mm³, or at least 800 mm³ in size. In other embodiments, the tumor is at least 1 cm long. With regard to a solid tumor, an established tumor generally has a newly established and robust blood supply and may have induced the regulatory T cells (Tregs) and myeloid derived suppressor cells (MDSC).

Unit dose: A discrete amount of the pharmaceutical composition comprising a predetermined amount of the active ingredient.

Vesicle: A fluid filled sac. In some embodiments the vesicle is a sac comprising an amphiphilic substance. In some embodiments, the sac is a nanoparticle-based vesicle, which refers to a vesicle with a size or dimensions in the nanometer range. In some embodiments, a polymer vesicle is a vesicle that is formed from one or more polymers.

### Definitions

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to an alkyl which may be substituted or not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

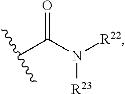

wherein R²² and R²³ each independently represent a hydrogen or hydrocarbyl group, or R²² and R²³ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

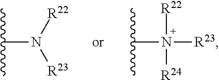

wherein R²², R²³, and R²⁴ each independently represent a hydrogen or a hydrocarbyl group, or R²² and R²³ taken together with the N atom to which they are attached complete a heterocycle having from 4 to 8 atoms in the ring structure.

The term “aminoalkyl”, as used herein, refers to an alkyl group substituted with an amino group. The term “aralkyl”, as used herein, refers to an alkyl group substituted with an aryl group. The term “aryl” as used herein includes substituted or unsubstituted aromatic carbocycles as well as heteroaryls. The term “aryl” is used interchangeably with the term “aromatic group” herein. Unless specifically stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, N(R^{a})C(O)N(R^{a})₂, —N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl. Aromatic carbocycles include single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

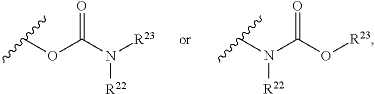

wherein R²² and R²³ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. For example, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR²² wherein R²² represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

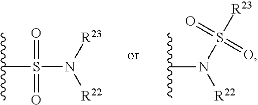

wherein R²² and R²³ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group. The term “thioester”, as used herein, refers to a group —C(O)SR²² or —SC(O)R²² wherein R²² represents a hydrocarbyl.

The term “thioether”, as used herein, is equivalent to an ether, wherein the oxygen is replaced with a sulfur.

The term “urea” is art-recognized and may be represented by the general formula

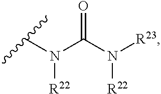

wherein R²² and R²³ independently represent hydrogen or a hydrocarbyl.

The term “aromatic amino acid” includes amino acids with a side chain comprising an aromatic group, such as phenylalanine, tyrosine, or tryptophan. Aromatic group refers to the portion of a molecule that comprises an aromatic ring. For example, phenylalanine is an aromatic amino acid that comprises an aromatic group, i.e., benzyl group. Phenylalanine (Phe) and Tryptophan (Trp) are prototypical aromatic amino acids.

A person of ordinary skill in the art would recognize that the definitions provided above are not intended to include impermissible substitution patterns (e.g., methyl substituted with 5 different groups, and the like). Such impermissible substitution patterns are easily recognized by a person of ordinary skill in the art. Any functional group disclosed herein and/or defined above can be substituted or unsubstituted, unless otherwise indicated herein. Unless otherwise explained, all technical and scientific terms used herein have the same meaning as commonly understood by one of ordinary skill in the art to which this disclosure belongs. The term “comprises” means “includes.” Therefore, comprising “A” or “B” refers to including A, including B, or including both A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described herein. In case of conflict, the present specification, including explanations of terms, will control. In addition, the materials, methods, and examples are illustrative only and not intended to be limiting.

### DESCRIPTION OF EMBODIMENTS

Provided herein are compositions of particles comprising amphiphiles and drug molecules useful for the treatment or prevention of a disease, e.g., cancer(s), autoimmune disease(s), allergy(ies) and/or infectious disease(s). Particles comprising certain compositions of amphiphiles and peptide antigen conjugates have particular utility for use as vaccines for treating or preventing disease, such as preventing or treating cancer(s), autoimmune disease(s), allergy(ies) and/or infectious disease(s).

The present disclosure relates to a vaccine comprising an amphiphile having the formula S-[B]-[U]-H; and at least one peptide antigen conjugate having the formula selected from [S]-[E1]-A-[E2]-[U]-H and H-[U]-[E1]-A-[E2]-[S],
wherein S, independently for each occurrence, is a solubilizing block;
B is a spacer;
H, independently for each occurrence, is a hydrophobic block,
   wherein one or more drug molecules (D) are optionally attached to each H directly or via a suitable linker X1;
A, independently for each occurrence, is a peptide antigen;
E1, independently for each occurrence, is an N-terminal extension;
E2, independently for each occurrence, is a C-terminal extension;
U, independently for each occurrence, is a linker;
[ ] denotes that the group is optional; and
— denotes that the two adjacent groups are directly attached to one another by a covalent bond or indirectly to one another via a suitable linker X,
wherein the amphiphile and/or the at least one peptide antigen conjugate comprises a dendron amplifier.

In some embodiments of vaccines, the amphiphile comprises a dendron amplifier. In other embodiments, the at least one peptide antigen conjugate comprises a dendron amplifier.

In some embodiments of vaccines, the S of the amphiphile comprises a dendron amplifier. In other embodiments, the S of the amphiphile has a dendritic architecture.

In some embodiments of vaccines, the S of the amphiphile comprises two or more solubilizing groups (SGs). In other embodiments, the two or more SGs are connected to the remaining portion of the S by a dendron amplifier, e.g., 4 to 8 SGs are connected to the S.

In some embodiments of vaccines, the SGs are independently selected from amines, hydroxyls, carboxylic acids and/or sugar molecules, wherein the sugar molecules are independently selected from mannose, glucose, glucosamine, N-acetyl glucose, galactose, galactosamine, N-acetyl galactosamine, phosphoserine and any derivatives thereof, agonists of CD22a, sialyl lewix x, and combinations thereof.

In some embodiments of vaccines, the dendron amplifier comprises repeating monomer units of 1 to 10 generations having between 2 to 6 branches per generation. In other embodiments, the dendron amplifier comprises repeating monomer units of 2 to 3 generations having between 2 to 3 branches per generation. In some embodiments of vaccines, the repeating monomer units are selected from FG1-(CH₂)_{y2}CH(R¹)₂, FG1-(CH₂)_{y2}C(R¹)₃, FG1-(CH₂CH₂O)_{y2}CH(R¹)₂, FG1-(CH₂CH₂O)_{y2}C(R¹)₃, and FG1-CH(R¹)₂, FG1-C(R¹)₃, wherein R₁, independently for each occurrence, is selected from (CH₂)_{y3}-FG2, (OCH₂CH₂)_{y3}-FG2, and CH₂(OCH₂CH₂)_{y3}-FG2); y2 and y3, independently for each occurrence, are each an integer of repeating units from 1 to 6; FG1 is a first functional group; and FG2 is a second functional group. In some embodiments, FG1 is —NH₂; and FG2, independently for each occurrence, is —CO₂— or —CO₂H. In some embodiments, FG1, independently for each occurrence, is —CO₂— or —CO₂H; and FG2 is —NH₂.

In some embodiments of vaccines, the SGs are linked to S via a suitable linker X5. In some embodiments of vaccines, the suitable linker X5 that links the SGs to S is selected from lower alkyl and PEG groups. In some embodiments of vaccines, two or more SGs are connected to the remaining portion of the S by a dendron amplifier through a suitable linker X5, which links the two or more SGs to a terminal functional (FGt) group of the dendron amplifier through an amide bond. In some embodiments, the linker X5 joining the SGs to the dendron amplifier is selected from —NH—R¹⁹, —NH—C(O)—R¹⁹, —C(O)—NH—R¹⁹— or —C(O)—R¹⁹, wherein R¹⁹ may be selected from but is not limited to —(CH₂)ₜ—, —(CH₂CH₂O)ₜ—CH₂CH₂—, —(CH₂)ₜ—C(O)—NH—(CH₂)ᵤ—, —(CH₂CH₂O)ₜCH₂CH₂C(O)—NH—(CH₂)ᵤ—, —(CH₂)ₜ—NH—C(O)—NH—(CH₂)ᵤ—, or —(CH₂CH₂O)ₜCH₂CH₂NH—C(O)—(CH₂)ᵤ— where t and u are each independently an integer typically selected from between 1 to 6, such as 1, 2, 3, 4, 5 or 6.

In some embodiments of vaccines, the dendron amplifier comprises a polyethylene oxide (PEG) group.

In some embodiments of vaccines, the H of the amphiphile comprises a higher alkane, an aromatic group, a fatty acid, a sterol, a polyunsaturated hydrocarbon, squalene, saponins, and/or a polymer.

In some embodiments of vaccines, the H of the peptide antigen conjugate comprises a higher alkane, an aromatic group, fatty acid, a sterol, a polyunsaturated hydrocarbon, and/or a polymer.

In some embodiments of vaccines, each H independently comprises a poly(amino acid) comprising monomers selected from hydrophobic amino acids (M), reactive amino acids (N), spacer amino acids (O), charged amino acids (P) and combinations thereof provided that at least one of M or N is present.

In some embodiments of vaccines, each H independently comprises a poly(amino acid) having the formula:

-(M)ₘ-(N)ₙ—(O)ₒ—(P)ₚ—R³

wherein M, N, O and P are each independently present or absent, provided that at least one of M or N is present;
m, n, o and p each independently denote an integer of 1 to 100 with the sum of m, n, o and p less than or equal to 100;
R³ is selected from hydrogen, NH₂, NH—CH₃, NH—(CH₂)_{y5}CH₃, OH or a drug molecule (D) either connected directly or through a suitable linker X1; and
y5 is an integer selected from 1 to 6.

In some embodiments of vaccines, P is absent. In other embodiments, N, O, and P are each absent.

In some embodiments of vaccines, P is

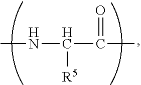

wherein each R⁵, independently, is a group that comprises 1 to 2 charged functional groups.

In some embodiments of vaccines, O is

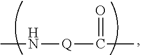

wherein each Q, independently, is selected from (CH₂)_{y6} and (CH₂CH₂O)_{y7}CH₂CH₂; each y6 is independently selected from an integer from 1 to 6; and each y7 is independently selected from an integer from 1 to 4.

In some embodiments of vaccines, N is

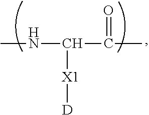

wherein each X1, independently, is a suitable linker; and each D, independently, is a drug molecule. In some embodiments of vaccines, X1 is absent. In other embodiments, X1 is present and is selected from lower alkyl and PEG groups. In other embodiments, X1 is present and is selected from an enzyme cleavable linker and a pH sensitive linker. In some embodiments of vaccines, X1 is present and comprise as enzyme degradable peptide and/or a self-immolative linker.

In some embodiments X1 is present and selected from —(CH₂)_{y10}-W and —(CH₂)_{y10}-R⁶, wherein y10 is an integer selected from 1 to 6, and R⁶ is selected from any one or more of —C(O)—NH—R⁷, —NH—C(O)—R⁷, —NH—C(O)—O—R⁷, —O—C(O)—NH—R⁷, —O—C(O)—R⁷, —C(O)—O—R⁷, —O—R⁷, O—C(O)—W, or —C(O)—W, wherein R⁷ is selected from any one or more of —(CH₂)_{y11}—W, —(CH₂)_{y11}—(OCH₂CH₂)_{y12}—W, —(CH₂)_{y13}—(OCH₂CH₂)_{y12}—(CH₂)_{y13}—W, —CHR⁸—C(O)—W, —CHR⁸—C(O)—(NH—CHR⁸—C(O))ⱼ—W, —(CH₂)_{y11}, —C(O)—NH—CHR⁸—C(O)—W, —(CH₂)_{y1}, —C(O)—NH—CHR⁸—C(O)—(NH—CHR⁸—C(O))ⱼ—W, —(CH₂)_{y11}—(OCH₂CH₂)_{y12}—C(O)—NH—CHR⁸—C(O)—W, —(CH₂)_{y11}—(OCH₂CH₂)_{y12}—(CH₂)_{y13}C(O)—NH—CHR⁸—C(O)—W, —(CH₂)_{y11}—(OCH₂CH₂)_{y12}—C(O)—NH—CHR⁸—C(O)—(NH—CHR⁸—C(O))ⱼ—W, —(CH₂)_{y11}—(OCH₂CH₂)_{y12}—(CH₂)_{y13}—C(O)—NH—CHR⁸—C(O)—(NH—CHR⁸—C(O))ⱼ—W, —CHR⁸—C(O)—NH—C₆H₄—CH₂—O—C(O)—W, —CHR⁸—C(O)—NH(CH₃)(CH₂)₂—O—C(O)—W, —CHR⁸—C(O)—(NH—CHR⁸—C(O))ⱼ—NH—C₆H₄—CH₂—O—C(O)—W, —CHR⁸—C(O)—(NH—CHR⁸—C(O))ⱼ—NH(CH₃)(CH₂)₂—O—C(O)—W, —(CH₂)_{y11}—C(O)—(NH—CHR⁸—C(O))ⱼ—NH—C₆H₄—CH₂—O—C(O)—W, —(CH₂)_{y11}—C(O)—(NH—CHR⁸—C(O))ⱼ—NH(CH₃)(CH₂)₂—O—C(O)—W, —(CH₂)_{y11}—(OCH₂CH₂)_{y12}—C(O)—(NH—CHR⁸—C(O))ⱼ—NH—C₆H₄—CH₂—O—C(O)—W, —(CH₂)_{y11}—(OCH₂CH₂)_{y12}—C(O)—(NH—CHR⁸—C(O))ⱼ—NH(CH₃)(CH₂)₂—O—C(O)—W, —(CH₂)_{y11}—(OCH₂CH₂)_{y12}—(CH₂)_{y13}C(O)—(NH—CHR⁸—C(O))ⱼ—NH—C₆H₄—CH₂—O—C(O)—W, —(CH₂)_{y11}—(OCH₂CH₂)_{y12}—(CH₂)_{y13}C(O)—(NH—CHR⁸—C(O))ⱼ—NH(CH₃)(CH₂)₂—O—C(O)—W, —(CH₂)_{y11}—(OCH₂CH₂)_{y12}—(CH₂)_{y13}—C(O)—NH—(CH₂)_{y14}—C(O)—(NH—CHR⁸—C(O))ⱼ—NH-C₆H₄—CH₂—O—C(O)—W, —(CH₂)_{y11}—(OCH₂CH₂)_{y12}—(CH₂)_{y13}C(O)—NH—(CH₂)_{y14}—C(O)—(NH—CHR⁸—C(O))ⱼ—NH(CH₃)(CH₂)₂—O—C(O)—W, —(CH₂)_{y11}—(OCH₂CH₂)_{y12}—C(O)—NH—(CH₂)_{y14}—C(O)—(NH—CHR⁸—C(O))ⱼ—NH-C₆H₄—CH₂—O—C(O)—W, —(CH₂)_{y11}—(OCH₂CH₂)_{y12}—C(O)—NH—(CH₂)_{y14}—C(O)—(NH—CHR⁸—C(O))ⱼ—NH(CH₃)(CH₂)₂—O—C(O)—W, —CHR⁸—C(O)—NH—(CH₂)_{y15}—W, —CHR⁸—NH—C(O)—(CH₂)_{y15}—W, —CHR⁸—C(O)—(NH—CHR⁸—C(O))ⱼ—NH—(CH₂)_{y15}—W, —CHR⁸—NH—(C(O)—CHR⁸—NH)ⱼ—C(O)—(CH₂)_{y15}—W, where y11, y12, y13, y14, y15 and j are each independently selected from an integer selected from 1 to 6, R⁸ is any amino acid side group, and W can be independently selected from H (hydrogen), FG3, LG and w; wherein FG3 is any suitable functional group for attachment to a functional group (“FG4”) present on a drug molecule, which may be selected from, but not limited to, carboxylic acid, activated carboxylic acids (e.g., carbonylthiazolidine-2-thione (“TT”), NHS or nitrophenol esters), carboxylic acid anhydrides, amine and protected amines (e.g., tert-butyloxycarbonyl protected amine), OSi(CH₃), alkene, azide, alkyne, stained-alkyne, halogen (e.g., fluoride, chloride), olefins and endo cyclic olefins (e.g., allyl), CN, OH, and epoxy, hydrazines (including hydrazides), carbohydrazides, aldehydes, ketones, carbamates and activated carbamates, LG is any suitable leaving group, which may be selected from any suitable leaving group (e.g., NHS, TT, nitrophenol, etc.); and w is a group that results from the reaction of either FG4 with FG3 or the displacement of LG with FG4, and is typically selected from NH—, C(O)—, NH—C(O)—, C(O)—NH—, O—C(O)—NH—, C(O)—NH—N═C(CH₃)—, NH-N═C(CH₃)— or —C(CH₃)═N—NH—C(O)—, wherein w is always linked to D either directly (i.e. w-D) or indirectly via X3 (i.e., w-X3-D).

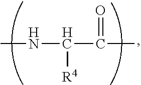

In some embodiments of vaccines, R⁴ is,

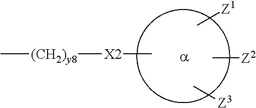

wherein
α is aryl or heteroaryl;
X2 is present or absent and when present is a suitable linker;
y8 is selected from an integer from 0 and 6; and
Z¹, Z², and Z³ are each independently selected from H, F, hydroxy, amino, alkyl, and fluoroalkyl.

In some embodiments of vaccines, a is an aryl, e.g., phenyl or naphthyl. In other embodiments, a is a heteroaryl, e.g., pyridinyl, quinolinyl, isoquinolinyl, indolyl, or benzimidazolyl.

In some embodiments of vaccines, X2 is absent. In other embodiments, X2 is present and is selected from C(O), CO₂(CH₂)_{y9}, CO₂, C(O)NH(CH₂)_{y9}, NHC(O) and NHC(O)(CH₂)_{y9}, wherein y9 is an integer typically selected from 1 to 6. In other embodiments, X2 is present and is selected from lower alkyl and PEG groups.

In some embodiments of vaccines, each R⁴ is independently selected from:

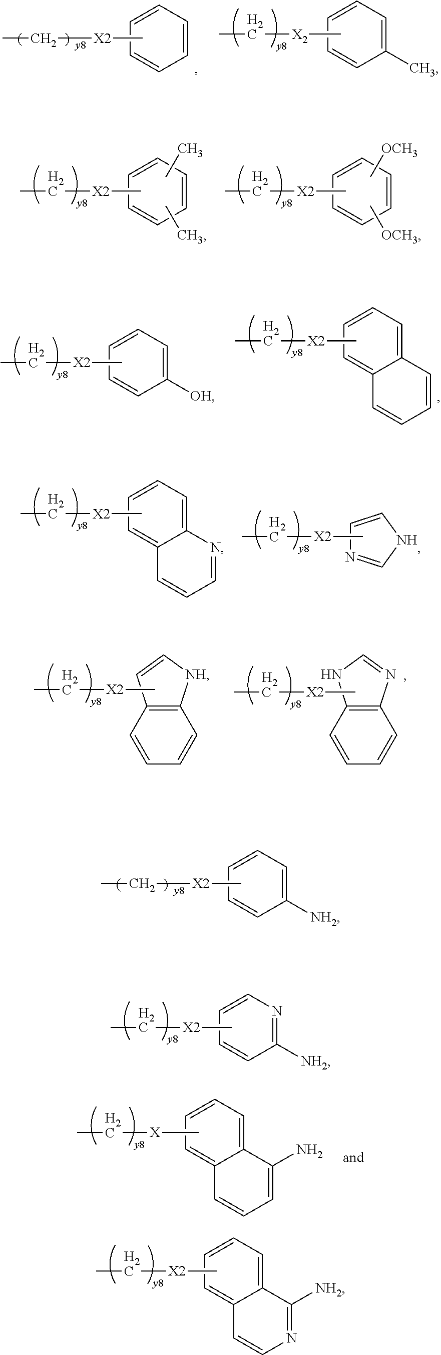

wherein each X2 is independently selected from a suitable linker and each y8 is independently selected from an integer from 0 and 6. In other embodiments, each R⁴ is independently selected from:

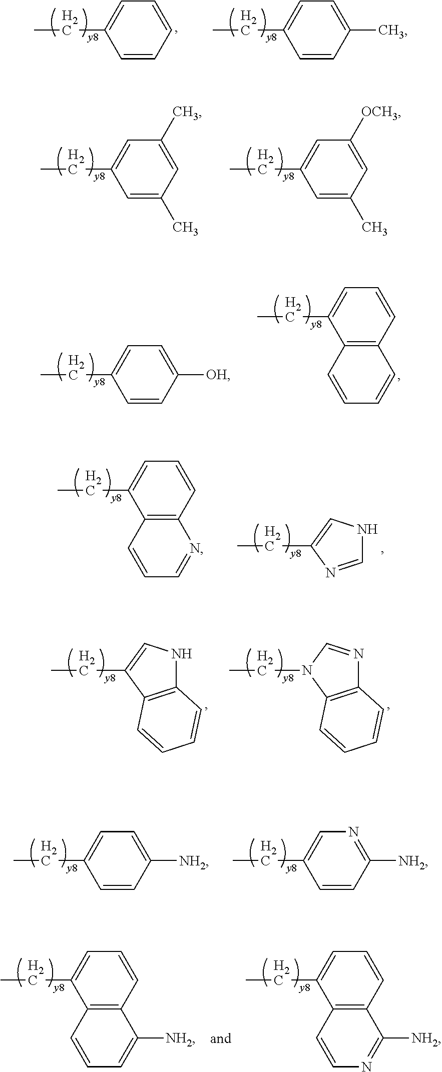

wherein each y8 is independently selected from an integer from 0 and 6. In other embodiments, each R⁴ is independently selected from:

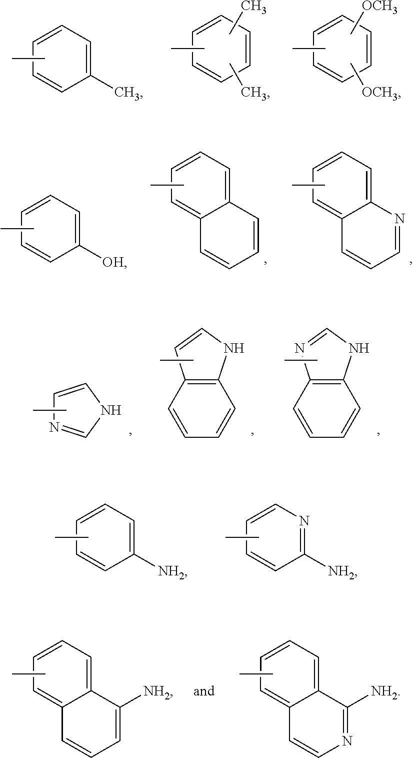

In other embodiments, each R⁴ is independently selected from:

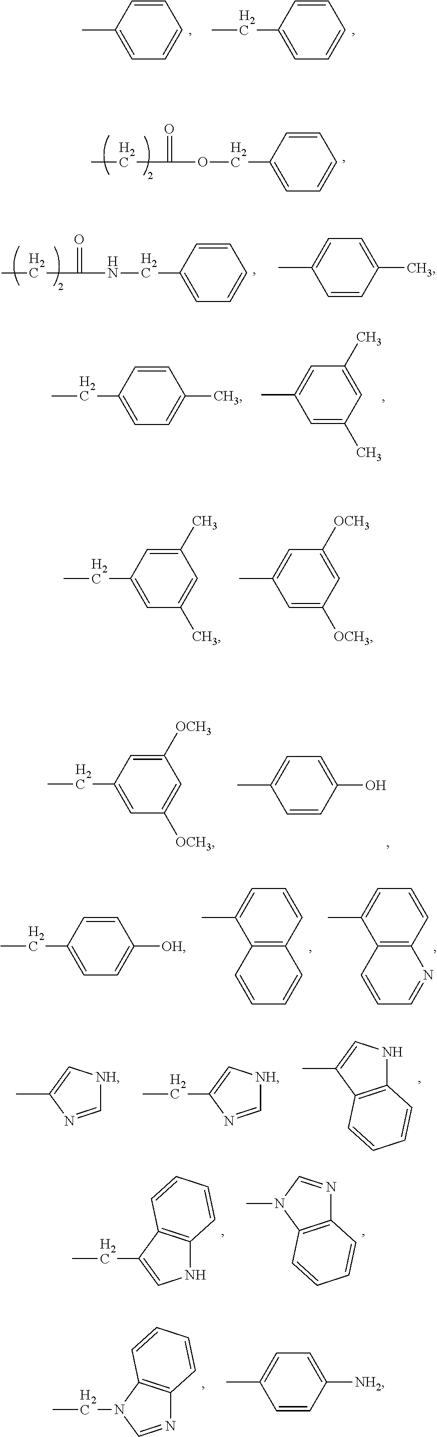

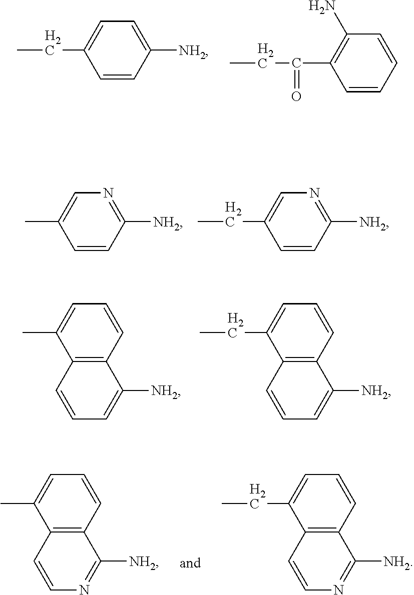

In preferred embodiments, each R⁴ is independently selected from:

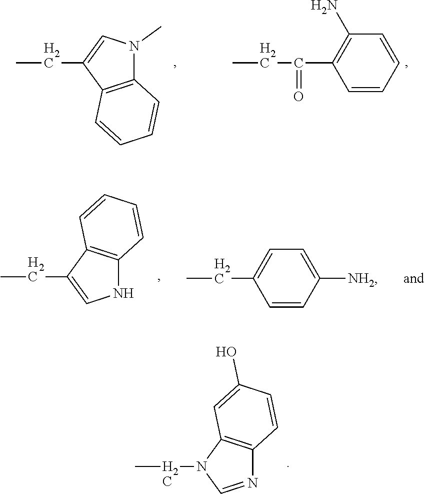

In some embodiments of vaccines, wherein at least one D is:
wherein,

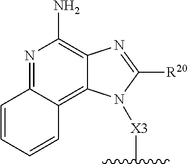

R²⁰ is selected from H, alkyl, alkoxyalkyl, aryl, heteroaryl, aminoalkyl, amide and ester; and X³ is selected from alkyl, alkoxyalkyl, aralkyl, heteroaralkyl, aryl, heteroaryl and carboxy.

In some embodiments of vaccines, R²⁰ is selected from H, alkyl and alkoxyalkyl; and X³ is selected from alkyl and aralkyl. In other embodiments, R²⁰ is butyl.

In some embodiments of vaccines, X³ is alkyl.

In some embodiments of vaccines, m, n, o and p each independently denote an integer of 1 to 30 with the sum of m, n, o and p less than or equal to 30.

In some embodiments of vaccines, m, n, o and p each independently denote an integer of 1 to 10 with the sum of m, n, o and p less than or equal to 10.

In some embodiments of vaccines, B is present and is a hydrophilic polymer, e.g., a PEG group. In other embodiments, B is present and is a hydrophilic peptide.

In some embodiments of vaccines, the PEG group comprises between 4 and 36 monomeric units. In other embodiments, the PEG group comprises between 4 and 12 monomeric units.

In some embodiments of vaccines, the hydrophilic peptide comprises between 4 and 36 amino acids. In other embodiments, the hydrophilic peptide comprises between 4 and 12 amino acids.

In some embodiments of vaccines, the amphiphile has the formula S-H. In other embodiments, the amphiphile has the formula S-B-U-H. In other embodiments, the amphiphile has the formula S-B-U-H-D.

In some embodiments of vaccines, the vaccine comprises a peptide antigen conjugate to amphiphile molar ratio of between about 4:1 to about 1:20.

In some embodiments of vaccines, the vaccine is a cancer vaccine, an infectious disease vaccine, a tolerance inducing allergy vaccine, a tolerance inducing autoimmune disease vaccine, a tolerance inducing transplant rejection vaccine, a cardiovascular vaccine or a neurodegenerative disease vaccine.

In some embodiments of vaccines, the peptide antigen (A) comprises a sequence wherein one or more cysteine residues have been replaced with alpha amino-butyric acid and/or one or more methionine residues have been replaced with norleucine. In some embodiments of vaccines, the at least one peptide antigen conjugate comprises an A is selected from minimal immunogens. Minimal immunogens are, for example, small peptide fragments derived from a naturally occurring protein that comprises a B cell epitope. Minimal immunogens can be used for cancer, infectious disease and tolerance inducing vaccines, as well as for the treatment of cardiovascular or neurodegenerative diseases.

In some embodiments of vaccines, A is a peptide antigen selected from RGYLTKILHVFHGLLPGFLVKMSGDLLE (SEQ ID NO: 106), PGFLVKMSGDLLE (SEQ ID NO: 107), PGFLVKnSGDLLE (SEQ ID NO:108), wherein n=norleucine; PGFLVKMSSDLLG (SEQ ID NO:109), PGFLVKnSSDLLG (SEQ ID NO:110), wherein n is norleucine; SIPWNLERITPPR (SEQ ID NO: 111); SIPWNLERITPPR (SEQ ID NO: 112); SIPWNLE (SEQ ID NO:113); SIPWNLEKVTPPR (SEQ ID NO:114); SIPWNLDRVTPPR (SEQ ID NO:115); NVPEEDGTRFHRQASKC (SEQ ID NO:116); NVPEEDGTRFHRQASK (SEQ ID NO:117); PEEDGTR (SEQ ID NO:118), NVPEEDG (SEQ ID NO:119); NVPEEDATRFHRQGSK (SEQ ID NO: 120); LFAPGEDIIGASSDCSTCFVSQSGTSQAAA (SEQ ID NO: 121); CSTCFVSQSGTSQAAA (SEQ ID NO:122); STCFVSQSGTSQAAA (SEQ ID NO:123), STBFVSQSGTSQAAA (SEQ ID NO: 124); STBFVSQ (SEQ ID NO: 125); MFTIKLLLFIVPLVISSRIDQDNSSFDSLSPEPKSRFAMLDDVKILANGLLQLGHGLKDFVHKT KGQIND (SEQ ID NO:126); EPKSRFAMLDDVKILANGLLQLGHGLKDFVHKTKGQIND (SEQ ID NO:127); EPKSRFAMLDDVKI (SEQ ID NO:128); MLDDVKILANGLLQ (SEQ ID NO: 129), LANGLLQLGHGLKD (SEQ ID NO:130); LGHGLKDFVHKTKG (SEQ ID NO:131); LKDFVHKTKGQIND (SEQ ID NO: 132); RFAMLDDVKILANGLLQLGH (SEQ ID NO: 133); GLLQLGHGLKDFVHKTKGQI (SEQ ID NO: 134); and IFQKLNIFDQSFYDLSLQTSEIKEEEKELRRTTYKLQVKNEEVKNMSLELNSKLESLLEEKILLQ QKVK (SEQ ID NO: 135).

In some embodiments of vaccines, A is directly attached by a covalent bond to an E1 that is directly attached by a covalent bond or indirectly via U to H.

In some embodiments of vaccines, A is directly attached by a covalent bond to an E2 that is directly attached by a covalent bond to or indirectly via U to H.

In some embodiments of vaccines, E1 and E2 each comprise a PEG group between 4 and 36 monomeric units, e.g., the PEG group comprises between 4 and 24 monomeric units.

In some embodiments of vaccines, E1 and E2 each comprise a peptide.

In some embodiments of vaccines, the peptide comprises 4 to 24 amino acids.

In some embodiments of vaccines, the peptide comprises amino acids selected from glycine, serine, threonine, alanine, and proline, e.g., the peptide is selected from (Gly-Ser)₂₋₁₂, (Gly-Gly-Gly-Gly-Ser)₁₋₄, and (Ala-Pro)₂₋₁₂.

In some embodiments of vaccines, the peptide comprises 7 to 28 amino acids.

In some embodiments of vaccines, the peptide comprises heptad repeats of formula (AA_{H}-AA_{P}-AA_{P}-AA_{H}-AA_{P}-AA_{P}-AA_{P})₁₋₄, wherein AA_{H} is a hydrophobic amino acid suitable for a coil domain selected from isoleucine, leucine, valine, and norleucine; and AA_{P} is a hydrophilic amino acid suitable for a coil domain selected from alanine, serine, lysine, aspartic acid, and glutamic acid, e.g. the peptide is selected from (Ile-Ala-Ala-Ile-Glu-Ser-Lys)₁₋₄, (Ile-Ala-Ala-Ile-Lys-Ser-Lys)₁₋₄, and (Ile-Ala-Ala-Ile-Glu-Ser-Glu)₁₋₄.

In some embodiments of vaccines, the at least one peptide antigen conjugate comprises an A selected from autoantigens, alloantigens, and allergens.

In some embodiments of vaccines, the S of the amphiphile comprises two or more solubilizing groups (SGs) independently selected from carboxylic acids, phosphoserine, and/or sugar molecules, wherein the sugar molecules are independently selected from mannose, glucose, glucosamine, N-acetyl glucose, galactose, galactosamine, and N-acetyl galactosamine, and agonists of CD22a.

In some embodiments of vaccines, the vaccine comprises at least one D selected from inhibitors of mTOR, RORγt, CDK8/19, and HDAC and agonists of AHR, RAR and A₂ₐ. In some embodiments of vaccines, the at least one D is selected from ATP-competitive mTOR inhibitors.

In some embodiments of vaccines, the vaccine further comprises a second drug molecule (D2) independently selected from inhibitors of mTOR, RORγt, CDK8/19, and HDACs, agonists of AHR, RAR and A₂ₐ, and immunostimulants selected from agonists of NLRs, CLRs, TLRs and STING, provided that D and D2 bind to different receptors.

In some embodiments of vaccines, the at least one D is selected from inhibitors of mTOR and agonists of AHR, and the D2 is selected from agonists of NLRs, CLRs, TLRs and STING. In some embodiments of vaccines, the at least one D is selected from ATP-competitive mTOR inhibitors and the D2 is selected from agonists of NLRs, CLRs, TLRs and STING.

In some embodiments of vaccines, the D2 is selected from agonists of TLR-3, TLR-7, TLR-8, TLR-7/8, TLR-9 and STING. In some embodiments of vaccines, the D2 is selected from RNA and imidazoquinoline agonists of TLR-7, TLR-8 and TLR-7/8.

In some embodiments of vaccines, the vaccine further comprises a third drug molecule (D3) independently selected from inhibitors of mTOR, RORγt, CDK8/19, and HDACs, agonists of AHR, RAR and A₂a, and immunostimulants selected from agonists of NLRs, CLRs, TLRs and STING, provided that D, D2 and D3 bind to different receptors.

In some embodiments of vaccines, wherein the at least one D is selected from AZD-8055, AZD-2016, KU-0063794, CC223, Torin-1, Torin-2, INK-128, WYE354, WYE132, OSI-027, OXA-01, PI-103, NVP-BEZ235, GNE-493, GSK2126458, rapamycin, tacrolimus, everolimus, RAD001, CCI-779 and AP23573.

In some embodiments of vaccines, wherein the molar ratio of total peptide antigen conjugate to the at least one D is between about 20:1 to 1:2, or about 10:1 to about 1:1 or about 4:1 to about 2:1.

In some embodiments of vaccines, the at least one peptide antigen conjugate comprises an A selected from tumor antigens.

In some embodiments of vaccines, the S of the amphiphile comprises two or more solubilizing groups (SGs) independently selected from amines or sugar molecules, wherein the sugar molecules are independently selected from mannose and sialyl lewix x, and combinations thereof. In some embodiments of vaccines, the S of the amphiphile comprises two or more solubilizing groups (SGs) independently selected from amines, carboxylic acids or sugar molecules, wherein the sugar molecules are independently selected from mannose, sialyl lewis x, sialyl lewis a, lewis y, lewis x, Tn, sTn, TF, sTF, Globo H, SSEA-3, GM2, GD2, GD3 and Fucosyl GM1 and combinations thereof.

In some embodiments of vaccines, each H of the amphiphile and/or the peptide antigen conjugate independently comprise a poly(amino acid) comprising monomers of hydrophobic amino acids (M) selected from tryptophan, 1-methyl tryptophan and para-amino phenylalanine. In other embodiments, each H of the amphiphile and/or the peptide antigen conjugate comprises a poly(amino acid) comprising monomers of the reactive amino acid (N), wherein the monomers comprise a D selected from a Glu-TLR-7/8a. In some embodiments of vaccines, at least one D is present and selected from agonists of TLR-3, TLR-7, TLR-8, TLR-7/8, TLR-9 and STING. In some embodiments of vaccines, the vaccine further comprises a second drug molecule (D2) selected from inhibitors of mTOR. In other embodiments, D2 is selected from rapamycin, tacrolimus, everolimus, RAD001, CCI-779 and AP23573. In some embodiments of vaccines, the molar ratio of peptide antigen conjugate to D2 is between about 20:1 to 1:2, or about 10:1 to about 1:1 or about 4:1 to about 2:1.

In some embodiments of vaccines, A is a glycopeptide. In other embodiments, A is selected from HGVT*S*APDT*RPAPGS*T*APPA (SEQ ID NO:319), DT*RPAPGS*T*APPAHGVT*S*AP (SEQ ID NO:320), GS*T*APPAHGVT*S*APDT*RPAPGS*T*APPA (SEQ ID NO:321), GVT*S*APDT*RPAP (SEQ ID NO:322), APDT*RPAPGS*T*A (SEQ ID NO:323), GS*T*APPAHGVT*S*AP (SEQ ID NO:324), VT*S*AP (SEQ ID NO:540), DT*RPAP (SEQ ID NO: 325) and GS*T*AP (SEQ ID NO:326), wherein * is an O-linked glycan and each occurrence is independently selected from sialyl lewis x, sialyl lewis a, lewis y, lewis x, Tn, sTn, TF, sTF.

In some embodiments of vaccines, S of the amphiphile comprises a second or third generation dendrimer; and B comprises from 4 to 36 PEG monomeric units.

In some embodiments of vaccines, S of the amphiphile comprises a second or third generation dendrimer; and H of the amphiphile comprises a poly(amino acid) comprising hydrophobic amino acids (M).

In some embodiments of vaccines, B comprises from 4 to 36 monomeric units; and H of the amphiphile comprises a poly(amino acid) comprising hydrophobic amino acids (M).

In some embodiments of vaccines, S of the amphiphile comprises a second or third generation dendrimer; B comprises from 4 to 36 PEG monomeric units; and H of the amphiphile comprises a poly(amino acid) comprising hydrophobic amino acids (M).

In some embodiments of vaccines, S of the amphiphile comprises a second or third generation dendrimer; and H of the amphiphile comprises a poly(amino acid) comprising para amino-phenylalanine.

In some embodiments of vaccines, B comprises from 4 to 36 PEG monomeric units; and H of the amphiphile comprises a polymer of para amino-phenylalanine.

In some embodiments of vaccines, S of the amphiphile comprises a second or third generation dendrimer; B comprises from 4 to 36 PEG monomeric units; and H of the amphiphile comprises a polymer of para amino-phenylalanine.

In some embodiments of vaccines, S of the amphiphile comprises a second or third generation dendrimer; and H of the amphiphile comprises a poly(amino acid) comprising hydrophobic amino acids (M) and reactive amino acids (N) that comprise an imidazoquinoline.

In some embodiments of vaccines, B comprises from 4 to 36 PEG monomeric units; and H of the amphiphile comprises a poly(amino acid) comprising hydrophobic amino acids (M) and reactive amino acids (N) that comprise an imidazoquinoline.

In some embodiments of vaccines, S of the amphiphile comprises a second or third generation dendrimer; B comprises from 4 to 36 PEG monomeric units; and H of the amphiphile comprises a poly(amino acid) comprising hydrophobic amino acids (M) and reactive amino acids (N) that comprise an imidazoquinoline.

In some embodiments of vaccines, S of the amphiphile comprises a second or third generation dendrimer; and H of the amphiphile comprises a poly(amino acid) of tryptophan and reactive amino acids (N) that comprise an imidazoquinoline.

In some embodiments of vaccines, B comprises from 4 to 36 PEG monomeric units; and H of the amphiphile comprises a poly(amino acid) of tryptophan and reactive amino acids (N) that comprise an imidazoquinoline.

In some embodiments of vaccines, S of the amphiphile comprises a second or third generation dendrimer; B comprises from 4 to 36 PEG monomeric units; and H of the amphiphile comprises a poly(amino acid) of tryptophan and reactive amino acids (N) that comprise an imidazoquinoline.

In some embodiments of vaccines, S of the amphiphile comprises a second or third generation dendrimer; B comprises from 4 to 36 PEG monomeric units; and SG comprises mannose.

In some embodiments of vaccines, S of the amphiphile comprises a second or third generation dendrimer; H of the amphiphile comprises a poly(amino acid) comprising hydrophobic amino acids (M); and SG comprises mannose.

In some embodiments of vaccines, B comprises from 4 to 36 PEG monomeric units; H of the amphiphile comprises a poly(amino acid) comprising hydrophobic amino acids (M); and SG comprises mannose.

In some embodiments of vaccines, S of the amphiphile comprises a second or third generation dendrimer; B comprises from 4 to 36 PEG monomeric units; H of the amphiphile comprises a poly(amino acid) comprising hydrophobic amino acids (M); and SG comprises mannose.

In some embodiments of vaccines, S of the amphiphile comprises a second or third generation dendrimer; H of the amphiphile comprises a polymer of para amino-phenylalanine; and SG comprises mannose.

In some embodiments of vaccines, B comprises from 4 to 36 PEG monomeric units; H of the amphiphile comprises a polymer of para amino-phenylalanine; and SG comprises mannose.

In some embodiments of vaccines, S of the amphiphile comprises a second or third generation dendrimer; B comprises from 4 to 36 PEG monomeric units; H of the amphiphile comprises a polymer of para amino-phenylalanine; and SG comprises mannose.

In some embodiments of vaccines, S of the amphiphile comprises a second or third generation dendrimer; and H of the amphiphile comprises a poly(amino acid) comprising hydrophobic amino acids (M) and reactive amino acids (N) that comprise an imidazoquinoline; and SG comprises mannose.

In some embodiments of vaccines, B comprises from 4 to 36 PEG monomeric units; and H of the amphiphile comprises a poly(amino acid) comprising hydrophobic amino acids (M) and reactive amino acids (N) that comprise an imidazoquinoline; and SG comprises mannose.

In some embodiments of vaccines, S of the amphiphile comprises a second or third generation dendrimer; B comprises from 4 to 36 PEG monomeric units; H of the amphiphile comprises a poly(amino acid) comprising hydrophobic amino acids (M) and reactive amino acids (N) that comprise an imidazoquinoline; and SG comprises mannose.

In some embodiments of vaccines, S of the amphiphile comprises a second or third generation dendrimer; and H of the amphiphile comprises a poly(amino acid) of tryptophan and reactive amino acids (N) that comprise an imidazoquinoline; and SG comprises mannose.

In some embodiments of vaccines, B comprises from 4 to 36 PEG monomeric units; and H of the amphiphile comprises a poly(amino acid) of tryptophan and reactive amino acids (N) that comprise an imidazoquinoline; and SG comprises mannose.

In some embodiments of vaccines, S of the amphiphile comprises a second or third generation dendrimer; B comprises from 4 to 36 PEG monomeric units; and H of the amphiphile comprises a poly(amino acid) of tryptophan and reactive amino acids (N) that comprise an imidazoquinoline; and SG comprises mannose.

In some embodiments of vaccines, S of the amphiphile comprises a second or third generation dendrimer; B comprises from 4 to 36 PEG monomeric units; and H of the peptide antigen conjugate comprises a poly(amino acid) comprising hydrophobic amino acids (M).

In some embodiments of vaccines, S of the amphiphile comprises a second or third generation dendrimer; H of the amphiphile comprises a poly(amino acid) comprising hydrophobic amino acids (M); and H of the peptide antigen conjugate comprises a poly(amino acid) comprising hydrophobic amino acids (M).

In some embodiments of vaccines, B comprises from 4 to 36 PEG monomeric units; H of the amphiphile comprises a poly(amino acid) comprising hydrophobic amino acids (M); and H of the peptide antigen conjugate comprises a poly(amino acid) comprising hydrophobic amino acids (M).

In some embodiments of vaccines, S of the amphiphile comprises a second or third generation dendrimer; B comprises from 4 to 36 PEG monomeric units; H of the amphiphile comprises a poly(amino acid) comprising hydrophobic amino acids (M); and H of the peptide antigen conjugate comprises a poly(amino acid) comprising hydrophobic amino acids (M).

In some embodiments of vaccines, S of the amphiphile comprises a second or third generation dendrimer; H of the amphiphile comprises a polymer of para amino-phenylalanine; and H of the peptide antigen conjugate comprises a poly(amino acid) comprising hydrophobic amino acids (M).

In some embodiments of vaccines, B comprises from 4 to 36 PEG monomeric units; H of the amphiphile comprises a polymer of para amino-phenylalanine; and H of the peptide antigen conjugate comprises a poly(amino acid) comprising hydrophobic amino acids (M).

In some embodiments of vaccines, S of the amphiphile comprises a second or third generation dendrimer; B comprises from 4 to 36 PEG monomeric units; H of the amphiphile comprises a polymer of para amino-phenylalanine; and H of the peptide antigen conjugate comprises a poly(amino acid) comprising hydrophobic amino acids (M).

n some embodiments of vaccines, S of the amphiphile comprises a second or third generation dendrimer; H of the amphiphile comprises a poly(amino acid) comprising hydrophobic amino acids (M) and reactive amino acids (N), that comprise an imidazoquinoline; and H of the peptide antigen conjugate comprises a poly(amino acid) comprising hydrophobic amino acids (M).

In some embodiments of vaccines, B comprises from 4 to 36 PEG monomeric units; H of the amphiphile comprises a poly(amino acid) comprising hydrophobic amino acids (M) and reactive amino acids (N), that comprise an imidazoquinoline; and H of the peptide antigen conjugate comprises a poly(amino acid) comprising hydrophobic amino acids (M).

In some embodiments of vaccines, S of the amphiphile comprises a second or third generation dendrimer; B comprises from 4 to 36 PEG monomeric units; H of the amphiphile comprises a poly(amino acid) comprising hydrophobic amino acids (M) and reactive amino acids (N) that comprise an imidazoquinoline; and H of the peptide antigen conjugate comprises a poly(amino acid) comprising hydrophobic amino acids (M).

In some embodiments of vaccines, S of the amphiphile comprises a second or third generation dendrimer; H of the amphiphile comprises a poly(amino acid) of tryptophan and reactive amino acids (N) that comprise an imidazoquinoline; and H of the peptide antigen conjugate comprises a poly(amino acid) comprising hydrophobic amino acids (M).

In some embodiments of vaccines, B comprises from 4 to 36 PEG monomeric units; H of the amphiphile comprises a poly(amino acid) of tryptophan and reactive amino acids (N) that comprise an imidazoquinoline; and H of the peptide antigen conjugate comprises a poly(amino acid) comprising hydrophobic amino acids (M).

In some embodiments of vaccines, S of the amphiphile comprises a second or third generation dendrimer; B comprises from 4 to 36 PEG monomeric units; H of the amphiphile comprises a poly(amino acid) of tryptophan and reactive amino acids (N) that comprise an imidazoquinoline; and H of the peptide antigen conjugate comprises a poly(amino acid) comprising hydrophobic amino acids (M).

In some embodiments of vaccines, S of the amphiphile comprises a second or third generation dendrimer; B comprises from 4 to 36 PEG monomeric units; SG comprises mannose; and H of the peptide antigen conjugate comprises a poly(amino acid) comprising hydrophobic amino acids (M).

In some embodiments of vaccines, S of the amphiphile comprises a second or third generation dendrimer; H of the amphiphile comprises a poly(amino acid) comprising hydrophobic amino acids (M); SG comprises mannose; and H of the peptide antigen conjugate comprises a poly(amino acid) comprising hydrophobic amino acids (M).

In some embodiments of vaccines, B comprises from 4 to 36 PEG monomeric units; H of the amphiphile comprises a poly(amino acid) comprising hydrophobic amino acids (M); SG comprises mannose; and H of the peptide antigen conjugate comprises a poly(amino acid) comprising hydrophobic amino acids (M).

In some embodiments of vaccines, S of the amphiphile comprises a second or third generation dendrimer; B comprises from 4 to 36 PEG monomeric units; H of the amphiphile comprises a poly(amino acid) comprising hydrophobic amino acids (M); SG comprises mannose; and H of the peptide antigen conjugate comprises a poly(amino acid) comprising hydrophobic amino acids (M).

In some embodiments of vaccines, S of the amphiphile comprises a second or third generation dendrimer; H of the amphiphile comprises a polymer of para amino-phenylalanine; SG comprises mannose; and H of the peptide antigen conjugate comprises a poly(amino acid) comprising hydrophobic amino acids (M).

In some embodiments of vaccines, B comprises from 4 to 36 PEG monomeric units; H of the amphiphile comprises a polymer of para amino-phenylalanine; SG comprises mannose; and H of the peptide antigen conjugate comprises a poly(amino acid) comprising hydrophobic amino acids (M).

In some embodiments of vaccines, S of the amphiphile comprises a second or third generation dendrimer; B comprises from 4 to 36 PEG monomeric units; H of the amphiphile comprises a polymer of para amino-phenylalanine; SG comprises mannose; and H of the peptide antigen conjugate comprises a poly(amino acid) comprising hydrophobic amino acids (M).

In some embodiments of vaccines, S of the amphiphile comprises a second or third generation dendrimer; and H of the amphiphile comprises a poly(amino acid) comprising hydrophobic amino acids (M) and reactive amino acids (N), that comprise an imidazoquinoline; SG comprises mannose; and H of the peptide antigen conjugate comprises a poly(amino acid) comprising hydrophobic amino acids (M).

In some embodiments of vaccines, B comprises from 4 to 36 PEG monomeric units; and H of the amphiphile comprises a poly(amino acid) comprising hydrophobic amino acids (M) and reactive amino acids (N) that comprise an imidazoquinoline; SG comprises mannose; and H of the peptide antigen conjugate comprises a poly(amino acid) comprising hydrophobic amino acids (M).

In some embodiments of vaccines, S of the amphiphile comprises a second or third generation dendrimer; B comprises from 4 to 36 PEG monomeric units; H of the amphiphile comprises a poly(amino acid) comprising hydrophobic amino acids (M) and reactive amino acids (N) that comprise an imidazoquinoline; SG comprises mannose; and H of the peptide antigen conjugate comprises a poly(amino acid) comprising hydrophobic amino acids (M).

In some embodiments of vaccines, S of the amphiphile comprises a second or third generation dendrimer; and H of the amphiphile comprises a poly(amino acid) of tryptophan and reactive amino acids (N) that comprise an imidazoquinoline; SG comprises mannose; and H of the peptide antigen conjugate comprises a poly(amino acid) comprising hydrophobic amino acids (M).

In some embodiments of vaccines, B comprises from 4 to 36 PEG monomeric units; H of the amphiphile comprises a poly(amino acid) of tryptophan and reactive amino acids (N) that comprise an imidazoquinoline; SG comprises mannose; and H of the peptide antigen conjugate comprises a poly(amino acid) comprising hydrophobic amino acids (M).

In some embodiments of vaccines, S of the amphiphile comprises a second or third generation dendrimer; B comprises from 4 to 36 PEG monomeric units; H of the amphiphile comprises a poly(amino acid) of tryptophan and reactive amino acids (N) that comprise an imidazoquinoline; SG comprises mannose; and H of the peptide antigen conjugate comprises a poly(amino acid) comprising hydrophobic amino acids (M).

In some embodiments of vaccines, S of the amphiphile comprises a second or third generation dendrimer; B comprises from 4 to 36 PEG monomeric units; the amphiphile comprises amino-hexanoic acid; and H of the peptide antigen conjugate comprises a poly(amino acid) comprising hydrophobic amino acids (M).

In some embodiments of vaccines, S of the amphiphile comprises a second or third generation dendrimer; H of the amphiphile comprises a poly(amino acid) comprising hydrophobic amino acids (M); the amphiphile comprises amino-hexanoic acid; and H of the peptide antigen conjugate comprises a poly(amino acid) comprising hydrophobic amino acids (M).

In some embodiments of vaccines, B comprises from 4 to 36 PEG monomeric units; H of the amphiphile comprises a poly(amino acid) comprising hydrophobic amino acids (M); the amphiphile comprises amino-hexanoic acid; and H of the peptide antigen conjugate comprises a poly(amino acid) comprising hydrophobic amino acids (M).

In some embodiments of vaccines, S of the amphiphile comprises a second or third generation dendrimer; B comprises from 4 to 36 PEG monomeric units; H of the amphiphile comprises a poly(amino acid) comprising hydrophobic amino acids (M); the amphiphile comprises amino-hexanoic acid; and H of the peptide antigen conjugate comprises a poly(amino acid) comprising hydrophobic amino acids (M).

In some embodiments of vaccines, S of the amphiphile comprises a second or third generation dendrimer; H of the amphiphile comprises a polymer of para amino-phenylalanine; the amphiphile comprises amino-hexanoic acid; and H of the peptide antigen conjugate comprises a poly(amino acid) comprising hydrophobic amino acids (M).

In some embodiments of vaccines, B comprises from 4 to 36 PEG monomeric units; H of the amphiphile comprises a polymer of para amino-phenylalanine; the amphiphile comprises amino-hexanoic acid; and H of the peptide antigen conjugate comprises a poly(amino acid) comprising hydrophobic amino acids (M).

In some embodiments of vaccines, S of the amphiphile comprises a second or third generation dendrimer; B comprises from 4 to 36 PEG monomeric units; H of the amphiphile comprises a polymer of para amino-phenylalanine; the amphiphile comprises amino-hexanoic acid; and H of the peptide antigen conjugate comprises a poly(amino acid) comprising hydrophobic amino acids (M).

In some embodiments of vaccines, S of the amphiphile comprises a second or third generation dendrimer; and H of the amphiphile comprises a poly(amino acid) comprising hydrophobic amino acids (M) and reactive amino acids (N), that comprise an imidazoquinoline; the amphiphile comprises amino-hexanoic acid; and H of the peptide antigen conjugate comprises a poly(amino acid) comprising hydrophobic amino acids (M).

In some embodiments of vaccines, B comprises from 4 to 36 PEG monomeric units; and H of the amphiphile comprises a poly(amino acid) comprising hydrophobic amino acids (M) and reactive amino acids (N) that comprise an imidazoquinoline; the amphiphile comprises amino-hexanoic acid; and H of the peptide antigen conjugate comprises a poly(amino acid) comprising hydrophobic amino acids (M).

In some embodiments of vaccines, S of the amphiphile comprises a second or third generation dendrimer; B comprises from 4 to 36 PEG monomeric units; H of the amphiphile comprises a poly(amino acid) comprising hydrophobic amino acids (M) and reactive amino acids (N) that comprise an imidazoquinoline; the amphiphile comprises amino-hexanoic acid; and H of the peptide antigen conjugate comprises a poly(amino acid) comprising hydrophobic amino acids (M).

In some embodiments of vaccines, S of the amphiphile comprises a second or third generation dendrimer; and H of the amphiphile comprises a poly(amino acid) of tryptophan and reactive amino acids (N) that comprise an imidazoquinoline; the amphiphile comprises amino-hexanoic acid; and H of the peptide antigen conjugate comprises a poly(amino acid) comprising hydrophobic amino acids (M).

In some embodiments of vaccines, B comprises from 4 to 36 PEG monomeric units; H of the amphiphile comprises a poly(amino acid) of tryptophan and reactive amino acids (N) that comprise an imidazoquinoline; the amphiphile comprises amino-hexanoic acid; and H of the peptide antigen conjugate comprises a poly(amino acid) comprising hydrophobic amino acids (M).

In some embodiments of vaccines, S of the amphiphile comprises a second or third generation dendrimer; B comprises from 4 to 36 PEG monomeric units; H of the amphiphile comprises a poly(amino acid) of tryptophan and reactive amino acids (N) that comprise an imidazoquinoline; the amphiphile comprises amino-hexanoic acid; and H of the peptide antigen conjugate comprises a poly(amino acid) comprising hydrophobic amino acids (M).

In some embodiments of vaccines, S of the amphiphile comprises a second or third generation dendrimer; B comprises from 4 to 36 PEG monomeric units; the dendrimer monomers comprise hydroxy acids and amino alcohols; and H of the peptide antigen conjugate comprises a poly(amino acid) comprising hydrophobic amino acids (M).

In some embodiments of vaccines, S of the amphiphile comprises a second or third generation dendrimer; H of the amphiphile comprises a poly(amino acid) comprising hydrophobic amino acids (M); the dendrimer monomers comprise hydroxy acids and amino alcohols; and H of the peptide antigen conjugate comprises a poly(amino acid) comprising hydrophobic amino acids (M).

In some embodiments of vaccines, B comprises from 4 to 36 PEG monomeric units; H of the amphiphile comprises a poly(amino acid) comprising hydrophobic amino acids (M); the dendrimer monomers comprise hydroxy acids and amino alcohols; and H of the peptide antigen conjugate comprises a poly(amino acid) comprising hydrophobic amino acids (M).

In some embodiments of vaccines, S of the amphiphile comprises a second or third generation dendrimer; B comprises from 4 to 36 PEG monomeric units; H of the amphiphile comprises a poly(amino acid) comprising hydrophobic amino acids (M); the dendrimer monomers comprise hydroxy acids and amino alcohols; and H of the peptide antigen conjugate comprises a poly(amino acid) comprising hydrophobic amino acids (M).

In some embodiments of vaccines, S of the amphiphile comprises a second or third generation dendrimer; H of the amphiphile comprises a polymer of para amino-phenylalanine; the dendrimer monomers comprise hydroxy acids and amino alcohols; and H of the peptide antigen conjugate comprises a poly(amino acid) comprising hydrophobic amino acids (M).

In some embodiments of vaccines, B comprises from 4 to 36 PEG monomeric units; H of the amphiphile comprises a polymer of para amino-phenylalanine; the dendrimer monomers comprise hydroxy acids and amino alcohols; and H of the peptide antigen conjugate comprises a poly(amino acid) comprising hydrophobic amino acids (M).

In some embodiments of vaccines, S of the amphiphile comprises a second or third generation dendrimer; B comprises from 4 to 36 PEG monomeric units; H of the amphiphile comprises a polymer of para amino-phenylalanine; the dendrimer monomers comprise hydroxy acids and amino alcohols; and H of the peptide antigen conjugate comprises a poly(amino acid) comprising hydrophobic amino acids (M).

In some embodiments of vaccines, S of the amphiphile comprises a second or third generation dendrimer; and H of the amphiphile comprises a poly(amino acid) comprising hydrophobic amino acids (M) and reactive amino acids (N), that comprise an imidazoquinoline; the dendrimer monomers comprise hydroxy acids and amino alcohols; and H of the peptide antigen conjugate comprises a poly(amino acid) comprising hydrophobic amino acids (M).

In some embodiments of vaccines, B comprises from 4 to 36 PEG monomeric units; and H of the amphiphile comprises a poly(amino acid) comprising hydrophobic amino acids (M) and reactive amino acids (N) that comprise an imidazoquinoline; the dendrimer monomers comprise hydroxy acids and amino alcohols; and H of the peptide antigen conjugate comprises a poly(amino acid) comprising hydrophobic amino acids (M).

In some embodiments of vaccines, S of the amphiphile comprises a second or third generation dendrimer; B comprises from 4 to 36 PEG monomeric units; H of the amphiphile comprises a poly(amino acid) comprising hydrophobic amino acids (M) and reactive amino acids (N) that comprise an imidazoquinoline; the dendrimer monomers comprise hydroxy acids and amino alcohols; and H of the peptide antigen conjugate comprises a poly(amino acid) comprising hydrophobic amino acids (M).

In some embodiments of vaccines, S of the amphiphile comprises a second or third generation dendrimer; and H of the amphiphile comprises a poly(amino acid) of tryptophan and reactive amino acids (N) that comprise an imidazoquinoline; the dendrimer monomers comprise hydroxy acids and amino alcohols; and H of the peptide antigen conjugate comprises a poly(amino acid) comprising hydrophobic amino acids (M).

In some embodiments of vaccines, B comprises from 4 to 36 PEG monomeric units; H of the amphiphile comprises a poly(amino acid) of tryptophan and reactive amino acids (N) that comprise an imidazoquinoline; the dendrimer monomers comprise hydroxy acids and amino alcohols; and H of the peptide antigen conjugate comprises a poly(amino acid) comprising hydrophobic amino acids (M).

In some embodiments of vaccines, S of the amphiphile comprises a second or third generation dendrimer; B comprises from 4 to 36 PEG monomeric units; H of the amphiphile comprises a poly(amino acid) of tryptophan and reactive amino acids (N) that comprise an imidazoquinoline; the dendrimer monomers comprise hydroxy acids and amino alcohols; and H of the peptide antigen conjugate comprises a poly(amino acid) comprising hydrophobic amino acids (M).

The present disclosure also relates to a vaccine for inducing tolerance comprising an amphiphile having the formula S-[B]-[U]-H; and at least one peptide antigen conjugate having the formula selected from [S]-[E1]-A-[E2]-[U]-H and H-[U]-[E1]-A-[E2]-[S],
wherein S, independently for each occurrence, is a solubilizing block;
B is a spacer;
H, independently for each occurrence, is a hydrophobic block,
   wherein one or more drug molecules (D) are optionally attached to each H directly or via a suitable linker X1;
A, independently for each occurrence, is a peptide antigen;
E1, independently for each occurrence, is an N-terminal extension;
E2, independently for each occurrence, is a C-terminal extension;
U, independently for each occurrence, is a linker;
[ ] denotes that the group is optional; and
— denotes that the two adjacent groups are directly attached to one another by a covalent bond or indirectly to one another via a suitable linker X,
wherein the amphiphile and/or the at least one peptide antigen conjugate comprises a dendron amplifier, and at least one peptide antigen is selected from an autoantigen, alloantigen, or allergen.

In some embodiments of vaccines for inducing tolerance, A is an autoantigen. In other embodiments, A is an allergen. In other embodiments, A is an alloantigen.

In some embodiments of vaccines for inducing tolerance, the amphiphile comprises a dendron amplifier. In other embodiments, the at least one peptide antigen conjugate comprises a dendron amplifier.

In some embodiments of vaccines for inducing tolerance, the S of the amphiphile comprises a dendron amplifier. In other embodiments, the S of the amphiphile has a dendritic architecture.

n some embodiments of vaccines for inducing tolerance, the S of the amphiphile comprises two or more solubilizing groups (SGs). In other embodiments, the two or more SGs are connected to the remaining portion of the S by a dendron amplifier, e.g., 4 to 8 SGs are connected to the S.

In some embodiments of vaccines for inducing tolerance, the SGs are independently selected from amines, hydroxyls, carboxylic acids and/or sugar molecules, wherein the sugar molecules are independently selected from mannose, glucose, glucosamine, N-acetyl glucose, galactose, galactosamine, N-acetyl galactosamine, phosphoserine and any derivatives thereof, agonists of CD22a, sialyl lewix x, and combinations thereof.

In some embodiments of vaccines for inducing tolerance, at least one SG is galactose. In other embodiments, at least one SG is phosphoserine. In other embodiments, at least one SG is an agonist of CD22a.

In some embodiments of vaccines for inducing tolerance, the dendron amplifier comprises repeating monomer units of 1 to 10 generations having between 2 to 6 branches per generation. In other embodiments, the dendron amplifier comprises repeating monomer units of 2 to 3 generations having between 2 to 3 branches per generation.

In some embodiments of vaccines for inducing tolerance, the repeating monomer units are selected from FG1-(CH₂)_{y2}CH(R¹)₂, FG1-(CH₂)_{y2}C(R¹)₃, FG1-(CH₂CH₂O)_{y2}CH(R¹)₂, FG1-(CH₂CH₂O)_{y2}C(R¹)₃, and FG1-CH(R¹)₂, FG1-C(R¹)₃, wherein R¹, independently for each occurrence, is selected from (CH₂)_{y3}-FG2, (OCH₂CH₂)_{y3}-FG2, and CH₂(OCH₂CH₂)_{y3}-FG2); y2 and y3, independently for each occurrence, is an integer of repeating units from 1 to 6; FG1 is a first functional group; and FG2 is a second functional group. In some embodiments, FG1 is —NH₂; and FG2, independently for each occurrence, is —CO₂— or —CO₂H. In some embodiments, FG1 is —CO₂— or —CO₂H; and FG2, independently for each occurrence, is —NH₂.

In some embodiments of vaccines for inducing tolerance, the SGs are linked to S via a suitable linker X5. In some embodiments of vaccines for inducing tolerance, the suitable linker X5 that links the SGs to S is selected from lower alkyl and PEG groups. In some embodiments of vaccines for inducing tolerance, two or more SGs are connected to the remaining portion of the S by a dendron amplifier through a suitable linker X5, which links the two or more SGs to a terminal functional (FGt) group of the dendron amplifier through an amide bond. In some embodiments of vaccine for inducing tolerance, the linker X5 joining the SGs to the dendron amplifier is selected from selected from —NH—R¹⁹, —NH—C(O)—R¹⁹, —C(O)—NH—R¹⁹— or —C(O)—R¹⁹, wherein R¹⁹ may be selected from but is not limited to —(CH₂)ₜ—, —(CH₂CH₂O)ₜ—CH₂CH₂—, —(CH₂)ₜ—C(O)—NH—(CH₂)ᵤ—, —(CH₂CH₂O)ₜCH₂CH₂C(O)—NH—(CH₂)ᵤ—, —(CH₂)ₜ—NH—C(O)—NH—(CH₂)ᵤ—, or —(CH₂CH₂O)ₜCH₂CH₂NH—C(O)—(CH₂)ᵤ— where t and u are each independently an integer typically selected from between 1 to 6, such as 1, 2, 3, 4, 5 or 6.

In some embodiments of vaccines for inducing tolerance, the dendron amplifier comprises a polyethylene oxide (PEG) group.

In some embodiments of vaccines for inducing tolerance, the H of the amphiphile comprises a higher alkane, an aromatic group, a fatty acid, a sterol, a polyunsaturated hydrocarbon, squalene, saponins, and/or a polymer.

In some embodiments of vaccines for inducing tolerance, the H of the peptide antigen conjugate comprises a higher alkane, an aromatic group, fatty acid, a sterol, a polyunsaturated hydrocarbon, and/or a polymer.

In some embodiments of vaccines for inducing tolerance, each H independently comprises a poly(amino acid) comprising monomers selected from hydrophobic amino acids (M), reactive amino acids (N), spacer amino acids (O), charged amino acids (P) and combinations thereof provided that at least one of M or N is present.

In some embodiments of vaccines for inducing tolerance, each H independently comprises a poly(amino acid) having the formula:

(M)ₘ-(N)ₙ—(O)ₒ—(P)ₚ—R³
wherein M, N, O and P are each independently present or absent, provided that at least one of M or N is present;
m, n, o and p each independently denote an integer of 1 to 100 with the sum of m, n, o and p less than or equal to 100;
R³ is selected from hydrogen, NH₂, NH—CH₃, NH—(CH₂)_{y5}CH₃, OH or a drug molecule (D) either connected directly or through a suitable linker X1; and
y5 is an integer selected from 1 to 6.

In some embodiments of vaccines for inducing tolerance, P is absent. In other embodiments, N, O, and P are each absent.

In some embodiments of vaccines for inducing tolerance, P is

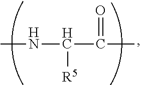

wherein each R⁵, independently, is a group that comprises 1 to 2 charged functional groups.

In some embodiments of vaccines for inducing tolerance, O is

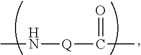

wherein each Q, independently, is selected from (CH₂)_{y} and (CH₂CH₂O); CH₂CH₂; each y is independently selected from an integer from 1 to 6; and each i is independently selected from an integer from 1 to 4.

In some embodiments of vaccines for inducing tolerance, N is

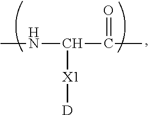

wherein each X1, independently, is a suitable linker; and each D, independently, is a drug molecule.

In some embodiments of vaccines for inducing tolerance, M is

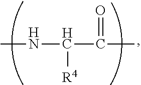

wherein each R⁴ is, independently, a hydrophobic group.

In some embodiments of vaccines for inducing tolerance, R⁴ is
wherein

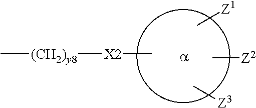

α is aryl or heteroaryl;
X2 is present or absent and when present is a suitable linker;
Y8 is selected from an integer from 0 and 6; and
Z¹, Z², and Z³ are each independently selected from H, F, hydroxy, amino, alkly, and fluoroalkyl.

In some embodiments of vaccines for inducing tolerance, a is an aryl, e.g., phenyl or naphthyl. In other embodiments, a is a heteroaryl, e.g., pyridinyl, quinolinyl, isoquinolinyl, indolyl, or benzimidazolyl.

In some embodiments of vaccines for inducing tolerance, X is absent. In other embodiments, X2 is present and is selected from C(O), CO₂(CH₂)_{y9}, CO₂, C(O)NH(CH₂)_{y9}, NHC(O) and NHC(O)(CH₂)_{y9}, wherein y9 is an integer typically selected from 1 to 6. In other embodiments, X2 is present and is selected from lower alkyl and PEG groups.

In some embodiments of vaccines for inducing tolerance, each R⁴ is independently selected from:

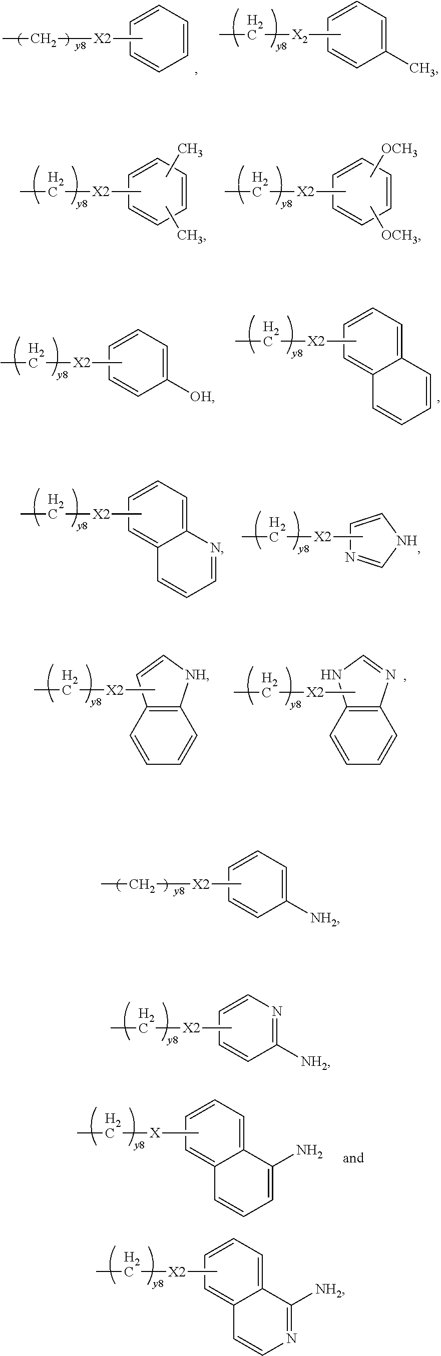

wherein each X2 is independently selected from a suitable linker and each y8 is independently selected from an integer from 0 and 6. In other embodiments, each R⁴ is independently selected from:

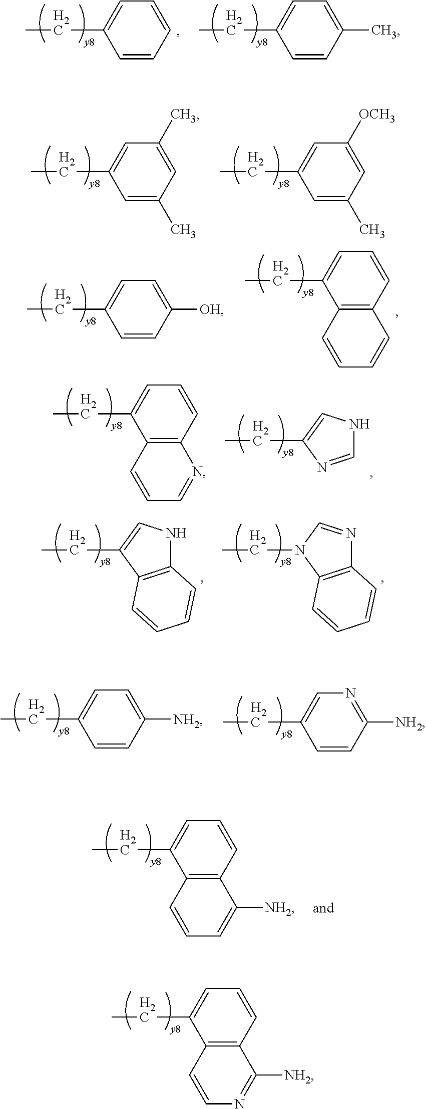

wherein each y8 is independently selected from an integer from 0 and 6. In other embodiments, each R⁴ is independently selected from:

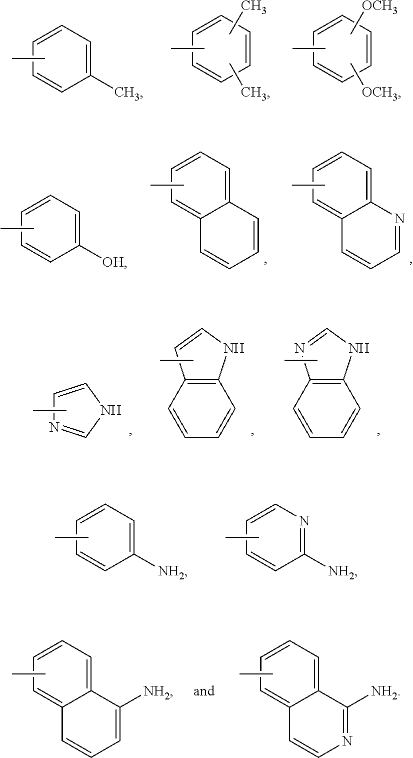

In other embodiments, each R⁴ is independently selected from:

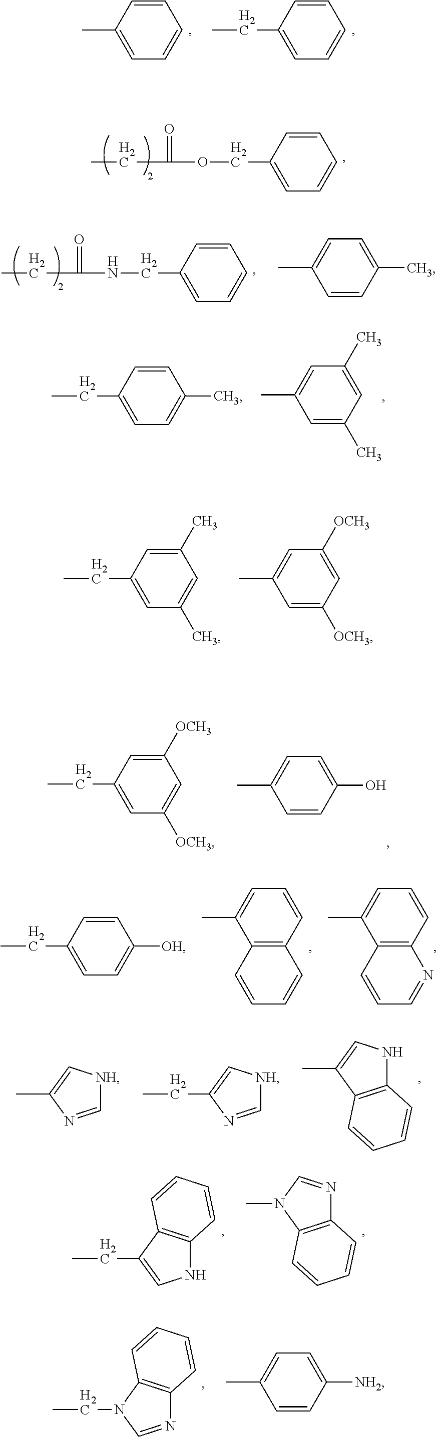

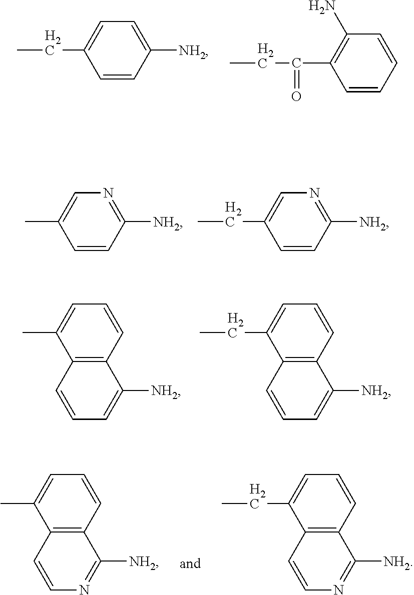

In preferred embodiments, each R⁴ is independently selected from:

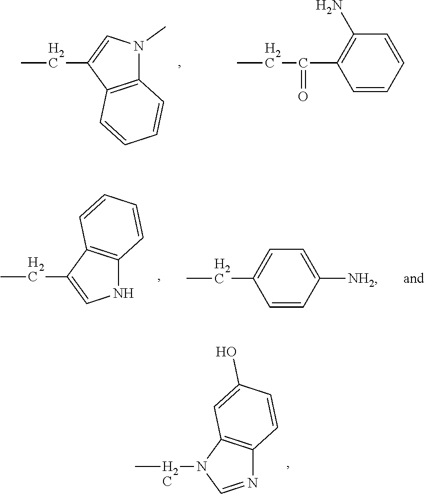

wherein y is selected from an integer from 1 and 6.

In some embodiments of vaccines for inducing tolerance, wherein at least one D is:
wherein,

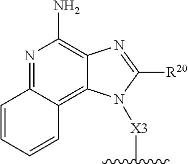

R²⁰ is selected from H, alkyl, alkoxyalkyl, aryl, heteroaryl, aminoalkyl, amide and ester; and X3 is selected from alkyl, alkoxyalkyl, aralkyl, heteroaralkyl, aryl, heteroaryl and carboxy.

In some embodiments of vaccines for inducing tolerance, R²⁰ is selected from H, alkyl and alkoxyalkyl; and X3 is selected from alkyl and aralkyl. In other embodiments, R²⁰ is butyl.

In some embodiments of vaccines for inducing tolerance, X3 is alkyl.

In some embodiments of vaccines for inducing tolerance, m, n, o and p each independently denote an integer of 1 to 30 with the sum of m, n, o and p less than or equal to 30.

In some embodiments of vaccines for inducing tolerance, m, n, o and p each independently denote an integer of 1 to 10 with the sum of m, n, o and p less than or equal to 10.

In some embodiments of vaccines for inducing tolerance, B is present and is a hydrophilic polymer, e.g., a PEG group. In other embodiments, B is present and is a hydrophilic peptide.

In some embodiments of vaccines for inducing tolerance, the PEG group comprises between 4 and 36 monomeric units. In other embodiments, the PEG group comprises between 4 and 12 monomeric units.

In some embodiments of vaccines for inducing tolerance, the hydrophilic peptide comprises between 4 and 36 amino acids. In other embodiments, the hydrophilic peptide comprises between 4 and 12 amino acids.

In some embodiments of vaccines for inducing tolerance, the amphiphile has the formula S-H. In other embodiments, the amphiphile has the formula S-B-U-H. In other embodiments, the amphiphile has the formula S-B-U-H-D.

In some embodiments of vaccines for inducing tolerance, the vaccine comprises a peptide antigen conjugate to amphiphile molar ratio of between about 4:1 to about 1:20.

n some embodiments of vaccines for inducing tolerance, the peptide antigen (A) comprises a sequence wherein one or more cysteine residues have been replaced with alpha amino-butyric acid and/or one or more methionine residues have been replaced with norleucine.

In some embodiments of vaccines for inducing tolerance, the vaccine comprises at least one D selected from inhibitors of mTOR, RORγt, CDK8/19, and HDAC and agonists of AHR, RAR and A₂a. In other embodiments, the at least one D is selected from ATP-competitive mTOR inhibitors.

In some embodiments of vaccines for inducing tolerance, the vaccine further comprises a second drug molecule (D2) independently selected from inhibitors of mTOR, RORγt, CDK8/19, and HDACs, agonists of AHR, RAR and A₂a, and immunostimulants selected from agonists of NLRs, CLRs, TLRs and STING, provided that D and D2 bind to different receptors.

In some embodiments of vaccines for inducing tolerance, the at least one D is selected from inhibitors of mTOR and agonists of AHR, and the D2 is selected from agonists of NLRs, CLRs, TLRs and STING.

In some embodiments of vaccines for inducing tolerance, wherein the at least one D is selected from ATP-competitive mTOR inhibitors, and the D2 is selected from agonists of NLRs, CLRs, TLRs and STING.

In other embodiments of vaccines for inducing tolerance, wherein the D2 is selected from agonists of TLR-3, TLR-7, TLR-8, TLR-7/8, TLR-9 and STING. In other embodiments of vaccines for inducing tolerance, wherein the D2 is selected from RNA and imidazoquinoline agonists of TLR-7, TLR-8 and TLR-7/8.

In some embodiments of vaccines for inducing tolerance, the vaccine further comprises a third drug molecule (D3) independently selected from inhibitors of mTOR, RORγt, CDK8/19, and HDACs, agonists of AHR, RAR and A₂ₐ, and immunostimulants selected from agonists of NLRs, CLRs, TLRs and STING, provided that D, D2 and D3 bind to different receptors.

In some embodiments of vaccines for inducing tolerance, the at least one D is selected from AZD-8055, AZD2016, KU-0063794, CC223, Torin-1, Torin-2, INK-128, WYE354, WYE132, OSI-027, OXA-01, PI-103, NVP-BEZ235, GNE-493, GSK2126458, rapamycin, tacrolimus, everolimus, RAD001, CCI-779 and AP23573.

In some embodiments of vaccines for inducing tolerance, the molar ratio of total peptide antigen conjugate to the at least one D is between about 20:1 to 1:2, or about 10:1 to about 1:1 or about 4:1 to about 2:1.

In some embodiments of vaccines for inducing tolerance, S of the amphiphile comprises a second or third generation dendrimer; and B comprises from 4 to 36 PEG monomeric units.

In some embodiments of vaccines for inducing tolerance, S of the amphiphile comprises a second or third generation dendrimer; and H of the amphiphile comprises a poly(amino acid) comprising hydrophobic amino acids (M).

In some embodiments of vaccines for inducing tolerance, B comprises from 4 to 36 PEG monomeric units; and H of the amphiphile comprises a poly(amino acid) comprising hydrophobic amino acids (M).

In some embodiments of vaccines for inducing tolerance, S of the amphiphile comprises a second or third generation dendrimer; B comprises from 4 to 36 PEG monomeric units; and H of the amphiphile comprises a poly(amino acid) comprising hydrophobic amino acids (M).

In some embodiments of vaccines for inducing tolerance, S of the amphiphile comprises a second or third generation dendrimer; and H of the amphiphile comprises a polymer of para amino-phenylalanine.

In some embodiments of vaccines for inducing tolerance, B comprises from 4 to 36 PEG monomeric units; and H of the amphiphile comprises a polymer of para amino-phenylalanine.

In some embodiments of vaccines for inducing tolerance, S of the amphiphile comprises a second or third generation dendrimer; B comprises from 4 to 36 PEG monomeric units; and H of the amphiphile comprises a polymer of para amino-phenylalanine.

In some embodiments of vaccines for inducing tolerance, S of the amphiphile comprises a second or third generation dendrimer; and H of the amphiphile comprises a poly(amino acid) comprising hydrophobic amino acids (M) and reactive amino acids (N) that comprise an imidazoquinoline.

In some embodiments of vaccines for inducing tolerance, B comprises from 4 to 36 PEG monomeric units; and H of the amphiphile comprises a poly(amino acid) comprising hydrophobic amino acids (M) and reactive amino acids (N) that comprise an imidazoquinoline.

In some embodiments of vaccines for inducing tolerance, S of the amphiphile comprises a second or third generation dendrimer; B comprises from 4 to 36 PEG monomeric units; and H of the amphiphile comprises a poly(amino acid) comprising hydrophobic amino acids (M) and reactive amino acids (N) that comprise an imidazoquinoline.

In some embodiments of vaccines for inducing tolerance, S of the amphiphile comprises a second or third generation dendrimer; and H of the amphiphile comprises a poly(amino acid) of tryptophan and reactive amino acids (N) that comprise an imidazoquinoline.

In some embodiments of vaccines for inducing tolerance, B comprises from 4 to 36 PEG monomeric units; and H of the amphiphile comprises a poly(amino acid) of tryptophan and reactive amino acids (N) that comprise an imidazoquinoline.

In some embodiments of vaccines for inducing tolerance, S of the amphiphile comprises a second or third generation dendrimer; B comprises from 4 to 36 PEG monomeric units; and H of the amphiphile comprises a poly(amino acid) of tryptophan and reactive amino acids (N) that comprise an imidazoquinoline.

In some embodiments of vaccines for inducing tolerance, S of the amphiphile comprises a second or third generation dendrimer; B comprises from 4 to 36 PEG monomeric units; and SG comprises N-acetyl galactosamine.

In some embodiments of vaccines for inducing tolerance, S of the amphiphile comprises a second or third generation dendrimer; H of the amphiphile comprises a poly(amino acid) comprising hydrophobic amino acids (M); and SG comprises N-acetyl galactosamine.

In some embodiments of vaccines for inducing tolerance, B comprises from 4 to 36 PEG monomeric units; H of the amphiphile comprises a poly(amino acid) comprising hydrophobic amino acids (M); and SG comprises N-acetyl galactosamine.

In some embodiments of vaccines for inducing tolerance, S of the amphiphile comprises a second or third generation dendrimer; B comprises from 4 to 36PEG monomeric units; H of the amphiphile comprises a poly(amino acid) comprising hydrophobic amino acids (M); and SG comprises N-acetyl galactosamine.

In some embodiments of vaccines for inducing tolerance, S of the amphiphile comprises a second or third generation dendrimer; H of the amphiphile comprises a polymer of para amino-phenylalanine; and SG comprises N-acetyl galactosamine.

In some embodiments of vaccines for inducing tolerance, B comprises from 4 to 36 PEG monomeric units; H of the amphiphile comprises a polymer of para amino-phenylalanine; and SG comprises N-acetyl galactosamine.

In some embodiments of vaccines for inducing tolerance, S of the amphiphile comprises a second or third generation dendrimer; B comprises from 4 to 36 PEG monomeric units; H of the amphiphile comprises a polymer of para amino-phenylalanine; and SG comprises N-acetyl galactosamine.

In some embodiments of vaccines for inducing tolerance, S of the amphiphile comprises a second or third generation dendrimer; and H of the amphiphile comprises a poly(amino acid) comprising hydrophobic amino acids (M) and reactive amino acids (N) that comprise an imidazoquinoline; and SG comprises N-acetyl galactosamine.

In some embodiments of vaccines for inducing tolerance, B comprises from 4 to 36 PEG monomeric units; and H of the amphiphile comprises a poly(amino acid) comprising hydrophobic amino acids (M) and reactive amino acids (N) that comprise an imidazoquinoline; and SG comprises N-acetyl galactosamine.

In some embodiments of vaccines for inducing tolerance, S of the amphiphile comprises a second or third generation dendrimer; B comprises from 4 to 36 PEG monomeric units; H of the amphiphile comprises a poly(amino acid) comprising hydrophobic amino acids (M) and reactive amino acids (N) that comprise an imidazoquinoline; and SG comprises N-acetyl galactosamine.

In some embodiments of vaccines for inducing tolerance, S of the amphiphile comprises a second or third generation dendrimer; and H of the amphiphile comprises a poly(amino acid) of tryptophan and reactive amino acids (N) that comprise an imidazoquinoline; and SG comprises N-acetyl galactosamine.

In some embodiments of vaccines for inducing tolerance, B comprises from 4 to 36 PEG monomeric units; and H of the amphiphile comprises a poly(amino acid) of tryptophan and reactive amino acids (N) that comprise an imidazoquinoline; and SG comprises N-acetyl galactosamine.

In some embodiments of vaccines for inducing tolerance, S of the amphiphile comprises a second or third generation dendrimer; B comprises from 4 to 36 PEG monomeric units; and H of the amphiphile comprises a poly(amino acid) of tryptophan and reactive amino acids (N) that comprise an imidazoquinoline; and SG comprises N-acetyl galactosamine.

In some embodiments of vaccines for inducing tolerance, S of the amphiphile comprises a second or third generation dendrimer; and B comprises from 4 to 36 PEG monomeric units; and the peptide antigen conjugate comprises an enzyme degradable linker.

In some embodiments of vaccines for inducing tolerance, S of the amphiphile comprises a second or third generation dendrimer; and H of the amphiphile comprises a poly(amino acid) comprising hydrophobic amino acids (M); and the peptide antigen conjugate comprises an enzyme degradable linker.

In some embodiments of vaccines for inducing tolerance, B comprises from 4 to 36 PEG monomeric units; and H of the amphiphile comprises a poly(amino acid) comprising hydrophobic amino acids (M); and the peptide antigen conjugate comprises an enzyme degradable linker.

In some embodiments of vaccines for inducing tolerance, S of the amphiphile comprises a second or third generation dendrimer; B comprises from 4 to 36 PEG monomeric units; H of the amphiphile comprises a poly(amino acid) comprising hydrophobic amino acids (M); and the peptide antigen conjugate comprises an enzyme degradable linker.

In some embodiments of vaccines for inducing tolerance, S of the amphiphile comprises a second or third generation dendrimer; and H of the amphiphile comprises a polymer of para amino-phenylalanine; and the peptide antigen conjugate comprises an enzyme degradable linker.

In some embodiments of vaccines for inducing tolerance, B comprises from 4 to 36 PEG monomeric units; and H of the amphiphile comprises a polymer of para amino-phenylalanine; and the peptide antigen conjugate comprises an enzyme degradable linker.

In some embodiments of vaccines for inducing tolerance, S of the amphiphile comprises a second or third generation dendrimer; B comprises from 4 to 36 PEG monomeric units; and H of the amphiphile comprises a polymer of para amino-phenylalanine; and the peptide antigen conjugate comprises an enzyme degradable linker.

In some embodiments of vaccines for inducing tolerance, S of the amphiphile comprises a second or third generation dendrimer; and H of the amphiphile comprises a poly(amino acid) comprising hydrophobic amino acids (M) and reactive amino acids (N), that comprise an imidazoquinoline; and the peptide antigen conjugate comprises an enzyme degradable linker.

In some embodiments of vaccines for inducing tolerance, B comprises from 4 to 36 PEG monomeric units; and H of the amphiphile comprises a poly(amino acid) comprising hydrophobic amino acids (M) and reactive amino acids (N), that comprise an imidazoquinoline; and the peptide antigen conjugate comprises an enzyme degradable linker.

In some embodiments of vaccines for inducing tolerance, S of the amphiphile comprises a second or third generation dendrimer; B comprises from 4 to 36 PEG monomeric units; and H of the amphiphile comprises a poly(amino acid) comprising hydrophobic amino acids (M) and reactive amino acids (N) that comprise an imidazoquinoline; and the peptide antigen conjugate comprises an enzyme degradable linker.

In some embodiments of vaccines for inducing tolerance, S of the amphiphile comprises a second or third generation dendrimer; and H of the amphiphile comprises a poly(amino acid) of tryptophan and reactive amino acids (N) that comprise an imidazoquinoline; and the peptide antigen conjugate comprises an enzyme degradable linker.

In some embodiments of vaccines for inducing tolerance, B comprises from 4 to 36 PEG monomeric units; and H of the amphiphile comprises a poly(amino acid) of tryptophan and reactive amino acids (N) that comprise an imidazoquinoline; and the peptide antigen conjugate comprises an enzyme degradable linker.

In some embodiments of vaccines for inducing tolerance, S of the amphiphile comprises a second or third generation dendrimer; B comprises from 4 to 36 PEG monomeric units; and H of the amphiphile comprises a poly(amino acid) of tryptophan and reactive amino acids (N) that comprise an imidazoquinoline; and the peptide antigen conjugate comprises an enzyme degradable linker.

In some embodiments of vaccines for inducing tolerance, S of the amphiphile comprises a second or third generation dendrimer; B comprises from 4 to 36 PEG monomeric units; and H of the peptide antigen conjugate comprises a poly(amino acid) comprising hydrophobic amino acids (M).

In some embodiments of vaccines for inducing tolerance, S of the amphiphile comprises a second or third generation dendrimer; H of the amphiphile comprises a poly(amino acid) comprising hydrophobic amino acids (M); and H of the peptide antigen conjugate comprises a poly(amino acid) comprising hydrophobic amino acids (M).

In some embodiments of vaccines for inducing tolerance, B comprises from 4 to 36 PEG monomeric units; H of the amphiphile comprises a poly(amino acid) comprising hydrophobic amino acids (M); and H of the peptide antigen conjugate comprises a poly(amino acid) comprising hydrophobic amino acids (M).

In some embodiments of vaccines for inducing tolerance, S of the amphiphile comprises a second or third generation dendrimer; B comprises from 4 to 36 PEG monomeric units; H of the amphiphile comprises a poly(amino acid) comprising hydrophobic amino acids (M); and H of the peptide antigen conjugate comprises a poly(amino acid) comprising hydrophobic amino acids (M).

In some embodiments of vaccines for inducing tolerance, S of the amphiphile comprises a second or third generation dendrimer; H of the amphiphile comprises a polymer of para amino-phenylalanine; and H of the peptide antigen conjugate comprises a poly(amino acid) comprising hydrophobic amino acids (M).

In some embodiments of vaccines for inducing tolerance, B comprises from 4 to 36 PEG monomeric units; H of the amphiphile comprises a polymer of para amino-phenylalanine; and H of the peptide antigen conjugate comprises a poly(amino acid) comprising hydrophobic amino acids (M).

In some embodiments of vaccines for inducing tolerance, S of the amphiphile comprises a second or third generation dendrimer; B comprises from 4 to 36 PEG monomeric units; H of the amphiphile comprises a polymer of para amino-phenylalanine; and H of the peptide antigen conjugate comprises a poly(amino acid) comprising hydrophobic amino acids (M).

In some embodiments of vaccines for inducing tolerance, S of the amphiphile comprises a second or third generation dendrimer; H of the amphiphile comprises a poly(amino acid) comprising hydrophobic amino acids (M) and reactive amino acids (N), that comprise an imidazoquinoline; and H of the peptide antigen conjugate comprises a poly(amino acid) comprising hydrophobic amino acids (M).

In some embodiments of vaccines for inducing tolerance, B comprises from 4 to 36 PEG monomeric units; H of the amphiphile comprises a poly(amino acid) comprising hydrophobic amino acids (M) and reactive amino acids (N), that comprise an imidazoquinoline; and H of the peptide antigen conjugate comprises a poly(amino acid) comprising hydrophobic amino acids (M).

In some embodiments of vaccines for inducing tolerance, S of the amphiphile comprises a second or third generation dendrimer; B comprises from 4 to 36 PEG monomeric units; H of the amphiphile comprises a poly(amino acid) comprising hydrophobic amino acids (M) and reactive amino acids (N) that comprise an imidazoquinoline; and H of the peptide antigen conjugate comprises a poly(amino acid) comprising hydrophobic amino acids (M).

In some embodiments of vaccines for inducing tolerance, S of the amphiphile comprises a second or third generation dendrimer; H of the amphiphile comprises a poly(amino acid) of tryptophan and reactive amino acids (N) that comprise an imidazoquinoline; and H of the peptide antigen conjugate comprises a poly(amino acid) comprising hydrophobic amino acids (M).

In some embodiments of vaccines for inducing tolerance, B comprises from 4 to 36 PEG monomeric units; H of the amphiphile comprises a poly(amino acid) of tryptophan and reactive amino acids (N) that comprise an imidazoquinoline; and H of the peptide antigen conjugate comprises a poly(amino acid) comprising hydrophobic amino acids (M).

In some embodiments of vaccines for inducing tolerance, wherein S of the amphiphile comprises a second or third generation dendrimer; B comprises from 4 to 36 PEG monomeric units; H of the amphiphile comprises a poly(amino acid) of tryptophan and reactive amino acids (N) that comprise an imidazoquinoline; and H of the peptide antigen conjugate comprises a poly(amino acid) comprising hydrophobic amino acids (M).

In some embodiments of vaccines for inducing tolerance, S of the amphiphile comprises a second or third generation dendrimer; B comprises from 4 to 36 PEG monomeric units; SG comprises N-acetyl galactosamine; and H of the peptide antigen conjugate comprises a poly(amino acid) comprising hydrophobic amino acids (M).

In some embodiments of vaccines for inducing tolerance, S of the amphiphile comprises a second or third generation dendrimer; H of the amphiphile comprises a poly(amino acid) comprising hydrophobic amino acids (M); SG comprises N-acetyl galactosamine; and H of the peptide antigen conjugate comprises a poly(amino acid) comprising hydrophobic amino acids (M).

In some embodiments of vaccines for inducing tolerance, B comprises from 4 to 36 PEG monomeric units; H of the amphiphile comprises a poly(amino acid) comprising hydrophobic amino acids (M); SG comprises N-acetyl galactosamine; and H of the peptide antigen conjugate comprises a poly(amino acid) comprising hydrophobic amino acids (M).

In some embodiments of vaccines for inducing tolerance, S of the amphiphile comprises a second or third generation dendrimer; B comprises from 4 to 36 PEG monomeric units; H of the amphiphile comprises a poly(amino acid) comprising hydrophobic amino acids (M); SG comprises N-acetyl galactosamine; and H of the peptide antigen conjugate comprises a poly(amino acid) comprising hydrophobic amino acids (M).

In some embodiments of vaccines for inducing tolerance, S of the amphiphile comprises a second or third generation dendrimer; H of the amphiphile comprises a polymer of para amino-phenylalanine; SG comprises N-acetyl galactosamine; and H of the peptide antigen conjugate comprises a poly(amino acid) comprising hydrophobic amino acids (M).

In some embodiments of vaccines for inducing tolerance, B comprises from 4 to 36 PEG monomeric units; H of the amphiphile comprises a polymer of para amino-phenylalanine; SG comprises N-acetyl galactosamine; and H of the peptide antigen conjugate comprises a poly(amino acid) comprising hydrophobic amino acids (M).

In some embodiments of vaccines for inducing tolerance, S of the amphiphile comprises a second or third generation dendrimer; B comprises from 4 to 36 PEG monomeric units; H of the amphiphile comprises a polymer of para amino-phenylalanine; SG comprises N-acetyl galactosamine; and H of the peptide antigen conjugate comprises a poly(amino acid) comprising hydrophobic amino acids (M).

In some embodiments of vaccines for inducing tolerance, S of the amphiphile comprises a second or third generation dendrimer; H of the amphiphile comprises a poly(amino acid) comprising hydrophobic amino acids (M) and reactive amino acids (N), that comprise an imidazoquinoline; and SG comprises N-acetyl galactosamine; and H of the peptide antigen conjugate comprises a poly(amino acid) comprising hydrophobic amino acids (M).

In some embodiments of vaccines for inducing tolerance, B comprises from 4 to 36 PEG monomeric units; H of the amphiphile comprises a poly(amino acid) comprising hydrophobic amino acids (M) and reactive amino acids (N) that comprise an imidazoquinoline; SG comprises N-acetyl galactosamine; and H of the peptide antigen conjugate comprises a poly(amino acid) comprising hydrophobic amino acids (M).

In some embodiments of vaccines for inducing tolerance, S of the amphiphile comprises a second or third generation dendrimer; B comprises from 4 to 36 PEG monomeric units; H of the amphiphile comprises a poly(amino acid) comprising hydrophobic amino acids (M) and reactive amino acids (N) that comprise an imidazoquinoline; SG comprises N-acetyl galactosamine; and H of the peptide antigen conjugate comprises a poly(amino acid) comprising hydrophobic amino acids (M).

In some embodiments of vaccines for inducing tolerance, S of the amphiphile comprises a second or third generation dendrimer; H of the amphiphile comprises a poly(amino acid) of tryptophan and reactive amino acids (N) that comprise an imidazoquinoline; SG comprises N-acetyl galactosamine; and H of the peptide antigen conjugate comprises a poly(amino acid) comprising hydrophobic amino acids (M).

In some embodiments of vaccines for inducing tolerance, B comprises from 4 to 36 PEG monomeric units; H of the amphiphile comprises a poly(amino acid) of tryptophan and reactive amino acids (N) that comprise an imidazoquinoline; SG comprises N-acetyl galactosamine; and H of the peptide antigen conjugate comprises a poly(amino acid) comprising hydrophobic amino acids (M).

In some embodiments of vaccines for inducing tolerance, S of the amphiphile comprises a second or third generation dendrimer; B comprises from 4 to 36 PEG monomeric units; H of the amphiphile comprises a poly(amino acid) of tryptophan and reactive amino acids (N) that comprise an imidazoquinoline; and SG comprises N-acetyl galactosamine; and H of the peptide antigen conjugate comprises a poly(amino acid) comprising hydrophobic amino acids (M).

In some embodiments of vaccines for inducing tolerance, S of the amphiphile comprises a second or third generation dendrimer; B comprises from 4 to 36 PEG monomeric units; the peptide antigen conjugate comprises an enzyme degradable linker; and H of the peptide antigen conjugate comprises a poly(amino acid) comprising hydrophobic amino acids (M).

In some embodiments of vaccines for inducing tolerance, S of the amphiphile comprises a second or third generation dendrimer; and H of the amphiphile comprises a poly(amino acid) comprising hydrophobic amino acids (M); the peptide antigen conjugate comprises an enzyme degradable linker; and H of the peptide antigen conjugate comprises a poly(amino acid) comprising hydrophobic amino acids (M).

In some embodiments of vaccines for inducing tolerance, B comprises from 4 to 36 PEG monomeric units; H of the amphiphile comprises a poly(amino acid) comprising hydrophobic amino acids (M); the peptide antigen conjugate comprises an enzyme degradable linker; and H of the peptide antigen conjugate comprises a poly(amino acid) comprising hydrophobic amino acids (M).

In some embodiments of vaccines for inducing tolerance, S of the amphiphile comprises a second or third generation dendrimer; B comprises from 4 to 36 PEG monomeric units; the amphiphile comprises amino-hexanoic acid; and H of the peptide antigen conjugate comprises a poly(amino acid) comprising hydrophobic amino acids (M).

In some embodiments of vaccines for inducing tolerance, S of the amphiphile comprises a second or third generation dendrimer; H of the amphiphile comprises a poly(amino acid) comprising hydrophobic amino acids (M); the amphiphile comprises amino-hexanoic acid; and H of the peptide antigen conjugate comprises a poly(amino acid) comprising hydrophobic amino acids (M).

In some embodiments of vaccines for inducing tolerance, B comprises from 4 to 36 PEG monomeric units; H of the amphiphile comprises a poly(amino acid) comprising hydrophobic amino acids (M); the amphiphile comprises amino-hexanoic acid; and H of the peptide antigen conjugate comprises a poly(amino acid) comprising hydrophobic amino acids (M).

In some embodiments of vaccines for inducing tolerance, S of the amphiphile comprises a second or third generation dendrimer; B comprises from 4 to 36 PEG monomeric units; H of the amphiphile comprises a poly(amino acid) comprising hydrophobic amino acids (M); the amphiphile comprises amino-hexanoic acid; and H of the peptide antigen conjugate comprises a poly(amino acid) comprising hydrophobic amino acids (M).

In some embodiments of vaccines for inducing tolerance, S of the amphiphile comprises a second or third generation dendrimer; H of the amphiphile comprises a polymer of para amino-phenylalanine; the amphiphile comprises amino-hexanoic acid; and H of the peptide antigen conjugate comprises a poly(amino acid) comprising hydrophobic amino acids (M).

In some embodiments of vaccines for inducing tolerance, B comprises from 4 to 36 PEG monomeric units; H of the amphiphile comprises a polymer of para amino-phenylalanine; the amphiphile comprises amino-hexanoic acid; and H of the peptide antigen conjugate comprises a poly(amino acid) comprising hydrophobic amino acids (M).

In some embodiments of vaccines for inducing tolerance, S of the amphiphile comprises a second or third generation dendrimer; B comprises from 4 to 36 PEG monomeric units; H of the amphiphile comprises a polymer of para amino-phenylalanine; the amphiphile comprises amino-hexanoic acid; and H of the peptide antigen conjugate comprises a poly(amino acid) comprising hydrophobic amino acids (M)

In some embodiments of vaccines for inducing tolerance, S of the amphiphile comprises a second or third generation dendrimer; H of the amphiphile comprises a poly(amino acid) comprising hydrophobic amino acids (M) and reactive amino acids (N) that comprise an imidazoquinoline; the amphiphile comprises amino-hexanoic acid; and H of the peptide antigen conjugate comprises a poly(amino acid) comprising hydrophobic amino acids (M).

In some embodiments of vaccines for inducing tolerance, B comprises from 4 to 36 PEG monomeric units; H of the amphiphile comprises a poly(amino acid) comprising hydrophobic amino acids (M) and reactive amino acids (N) that comprise an imidazoquinoline; the amphiphile comprises amino-hexanoic acid; and H of the peptide antigen conjugate comprises a poly(amino acid) comprising hydrophobic amino acids (M).

In some embodiments of vaccines for inducing tolerance, S of the amphiphile comprises a second or third generation dendrimer; B comprises from 4 to 36 PEG monomeric units; H of the amphiphile comprises a poly(amino acid) comprising hydrophobic amino acids (M) and reactive amino acids (N) that comprise an imidazoquinoline; the amphiphile comprises amino-hexanoic acid; and H of the peptide antigen conjugate comprises a poly(amino acid) comprising hydrophobic amino acids (M).

In some embodiments of vaccines for inducing tolerance, S of the amphiphile comprises a second or third generation dendrimer; H of the amphiphile comprises a poly(amino acid) of tryptophan and reactive amino acids (N) that comprise an imidazoquinoline; the amphiphile comprises amino-hexanoic acid; and H of the peptide antigen conjugate comprises a poly(amino acid) comprising hydrophobic amino acids (M).

In some embodiments of vaccines for inducing tolerance, B comprises from 4 to 36 PEG monomeric units; H of the amphiphile comprises a poly(amino acid) of tryptophan and reactive amino acids (N) that comprise an imidazoquinoline; the amphiphile comprises amino-hexanoic acid; and H of the peptide antigen conjugate comprises a poly(amino acid) comprising hydrophobic amino acids (M).

In some embodiments of vaccines for inducing tolerance, S of the amphiphile comprises a second or third generation dendrimer; B comprises from 4 to 36 PEG monomeric units; H of the amphiphile comprises a poly(amino acid) of tryptophan and reactive amino acids (N) that comprise an imidazoquinoline; the amphiphile comprises amino-hexanoic acid; and H of the peptide antigen conjugate comprises a poly(amino acid) comprising hydrophobic amino acids (M).

In some embodiments of vaccines for inducing tolerance, S of the amphiphile comprises a second or third generation dendrimer; B comprises from 4 to 36 PEG monomeric units; the dendrimer monomers comprise hydroxy acids and amino alcohols; and H of the peptide antigen conjugate comprises a poly(amino acid) comprising hydrophobic amino acids (M).

In some embodiments of vaccines for inducing tolerance, S of the amphiphile comprises a second or third generation dendrimer; H of the amphiphile comprises a poly(amino acid) comprising hydrophobic amino acids (M); the dendrimer monomers comprise hydroxy acids and amino alcohols; and H of the peptide antigen conjugate comprises a poly(amino acid) comprising hydrophobic amino acids (M).

In some embodiments of vaccines for inducing tolerance, B comprises from 4 to 36 PEG monomeric units; H of the amphiphile comprises a poly(amino acid) comprising hydrophobic amino acids (M); the dendrimer monomers comprise hydroxy acids and amino alcohols; and H of the peptide antigen conjugate comprises a poly(amino acid) comprising hydrophobic amino acids (M).

In some embodiments of vaccines for inducing tolerance, S of the amphiphile comprises a second or third generation dendrimer; B comprises from 4 to 36 PEG monomeric units; H of the amphiphile comprises a poly(amino acid) comprising hydrophobic amino acids (M); the dendrimer monomers comprise hydroxy acids and amino alcohols; and H of the peptide antigen conjugate comprises a poly(amino acid) comprising hydrophobic amino acids (M).

In some embodiments of vaccines for inducing tolerance, S of the amphiphile comprises a second or third generation dendrimer; H of the amphiphile comprises a polymer of para amino-phenylalanine; the dendrimer monomers comprise hydroxy acids and amino alcohols; and H of the peptide antigen conjugate comprises a poly(amino acid) comprising hydrophobic amino acids (M).

In some embodiments of vaccines for inducing tolerance, B comprises from 4 to 36 PEG monomeric units; H of the amphiphile comprises a polymer of para amino-phenylalanine; the dendrimer monomers comprise hydroxy acids and amino alcohols; and H of the peptide antigen conjugate comprises a poly(amino acid) comprising hydrophobic amino acids (M).

In some embodiments of vaccines for inducing tolerance, S of the amphiphile comprises a second or third generation dendrimer; B comprises from 4 to 36 PEG monomeric units; H of the amphiphile comprises a polymer of para amino-phenylalanine; the dendrimer monomers comprise hydroxy acids and amino alcohols; and H of the peptide antigen conjugate comprises a poly(amino acid) comprising hydrophobic amino acids (M).

In some embodiments of vaccines for inducing tolerance, S of the amphiphile comprises a second or third generation dendrimer; H of the amphiphile comprises a poly(amino acid) comprising hydrophobic amino acids (M) and reactive amino acids (N) that comprise an imidazoquinoline; the dendrimer monomers comprise hydroxy acids and amino alcohols; and H of the peptide antigen conjugate comprises a poly(amino acid) comprising hydrophobic amino acids (M).

In some embodiments of vaccines for inducing tolerance, B comprises from 4 to 36 PEG monomeric units; H of the amphiphile comprises a poly(amino acid) comprising hydrophobic amino acids (M) and reactive amino acids (N) that comprise an imidazoquinoline; the dendrimer monomers comprise hydroxy acids and amino alcohols; and H of the peptide antigen conjugate comprises a poly(amino acid) comprising hydrophobic amino acids (M).

In some embodiments of vaccines for inducing tolerance, S of the amphiphile comprises a second or third generation dendrimer; B comprises from 4 to 36 PEG monomeric units; H of the amphiphile comprises a poly(amino acid) comprising hydrophobic amino acids (M) and reactive amino acids (N) that comprise an imidazoquinoline; the dendrimer monomers comprise hydroxy acids and amino alcohols; and H of the peptide antigen conjugate comprises a poly(amino acid) comprising hydrophobic amino acids (M).

In some embodiments of vaccines for inducing tolerance, S of the amphiphile comprises a second or third generation dendrimer; H of the amphiphile comprises a poly(amino acid) of tryptophan and reactive amino acids (N) that comprise an imidazoquinoline; the dendrimer monomers comprise hydroxy acids and amino alcohols; and H of the peptide antigen conjugate comprises a poly(amino acid) comprising hydrophobic amino acids (M).

In some embodiments of vaccines for inducing tolerance, B comprises from 4 to 36 PEG monomeric units; H of the amphiphile comprises a poly(amino acid) of tryptophan and reactive amino acids (N) that comprise an imidazoquinoline; the dendrimer monomers comprise hydroxy acids and amino alcohols; and H of the peptide antigen conjugate comprises a poly(amino acid) comprising hydrophobic amino acids (M).

In some embodiments of vaccines for inducing tolerance, S of the amphiphile comprises a second or third generation dendrimer; B comprises from 4 to 36 PEG monomeric units; H of the amphiphile comprises a poly(amino acid) of tryptophan and reactive amino acids (N) that comprise an imidazoquinoline; the dendrimer monomers comprise hydroxy acids and amino alcohols; and H of the peptide antigen conjugate comprises a poly(amino acid) comprising hydrophobic amino acids (M).

The present disclosure also relates to a vaccine comprising an amphiphile having the formula S-[B]-[U]-H; and at least one peptide antigen conjugate having the formula selected from [S]-[E1]-A-[E2]-[U]-H and H-[U]-[E1]-A-[E2]-[S],
wherein S, independently for each occurrence, is a solubilizing block;
B is a spacer;
H, independently for each occurrence, is a hydrophobic block,
   wherein one or more drug molecules (D) are optionally attached to each H directly or via a suitable linker X1;
A, independently for each occurrence, is a peptide antigen;
E1, independently for each occurrence, is an N-terminal extension;
E2, independently for each occurrence, is a C-terminal extension;
U, independently for each occurrence, is a linker;
[ ] denotes that the group is optional; and
— denotes that the two adjacent groups are directly attached to one another by a covalent bond or indirectly to one another via a suitable linker X,
wherein the amphiphile and/or the at least one peptide antigen conjugate comprises a dendron amplifier, and
at least one A comprises a sequence wherein one or more cysteine residues have been replaced with alpha amino-butyric acid and/or one or more methionine residues have been replaced with norleucine.

In some embodiments of vaccines, S is present for at least one peptide antigen conjugate and comprises SGs selected from amines or carboxylic acids. In some embodiments of vaccines, the S comprises one or more lysine or ornithine residues.

In some embodiments of vaccines, the peptide antigen conjugate has a net positive charge between about +1 to about +10 at physiologic pH.

In some embodiments of vaccines, the peptide antigen conjugate has a net positive charge between about +2 to about +6 or between about +3 to about +5 at physiologic pH.

In some embodiments of vaccines, the S comprises one or more glutamic acid or aspartic acid residues.

In some embodiments of vaccines, the peptide antigen conjugate has a net negative charge between about −1 to about −10 at physiologic pH. In other embodiments of vaccines, the peptide antigen conjugate has a net negative charge between about −2 to about −6 or between about −3 to about −5 at physiologic pH.

In some embodiments of vaccines, the S of the amphiphile comprises carboxylic acids. In other embodiments of vaccines, the S of the amphiphile comprises succinic acid or beta alanine.

In some embodiments of vaccines, the molar ratio of peptide antigen conjugate to amphiphile is between about 4:1 to 1:20

In some embodiments of vaccines, the average net charge of the at least one peptide antigen conjugate is positive at physiologic pH and the molar ratio of peptide antigen conjugate to amphiphile is between about 4:1 to about 2:1 or about 1:2 to about 1:16, or about 1:2 to about 1:4.

The present disclosure also relates to a vaccine comprising at least one peptide antigen (A), wherein at least one peptide antigen (A) comprises a sequence wherein one or more cysteine residues have been replaced with alpha amino-butyric acid and/or one or more methionine residues have been replaced with norleucine.

In some embodiments of vaccines, the vaccine further comprises a particle delivery system selected from lipid emulsions, liposomes, PLGA particles, inorganic salt particles and metal nanoparticles. In other embodiments of vaccines, the vaccine further comprising at least one drug molecule (D) selected from immunostimulants and Treg promoting immunomodulators.

The present disclosure also relates to a vaccine comprising at least one peptide antigen conjugate having the formula selected from [S]-[E1]-A-[E2]-[U]-H and H-[U]-[E1]-A-[E2]-[S],
wherein S, independently for each occurrence, is a solubilizing block;
H, independently for each occurrence, is a hydrophobic block,
   wherein one or more drug molecules (D) are optionally attached to each H directly or via a suitable linker X1;
A, independently for each occurrence, is a peptide antigen;
E1, independently for each occurrence, is an N-terminal extension;
E2, independently for each occurrence, is a C-terminal extension;
U, independently for each occurrence, is a linker;
   wherein either:
      (i) at least one A comprises alpha amino-butyric acid and/or norleucine;
      (ii) at least one A is selected from tumor antigens, at least one D is present and is selected from agonists of TLR-7/8, and the vaccine further comprises a second drug molecule (D2) selected from inhibitors of mTOR;
      (iii) at least one A is a glycopeptide; or
      (iv) at least one A is selected from autoantigens, allergens and alloantigens and at least one D is present and is selected from ATP-competitive mTOR inhibitors; [ ] denotes that the group is optional; and
— denotes that the two adjacent groups are directly attached to one another by a covalent bond or indirectly to one another via a suitable linker X.

In some embodiments of vaccines, wherein the at least one peptide antigen conjugate comprises at least one A selected from tumor antigens.

In some embodiments of vaccines, wherein at least one D is selected from agonists of TLR-3, TLR-7, TLR-8, TLR-9, and STING

In some embodiments of vaccines, wherein each H of the amphiphile and/or the peptide antigen conjugate comprises a poly(amino acid) comprising monomers of the reactive amino acid (N), wherein the monomers comprise a D selected from agonists of TLR-7/8.

In some embodiments of vaccines, wherein D2 is selected from rapamycin, tacrolimus, everolimus, RAD001, CCI-779 and AP23573.

In some embodiments of vaccines, wherein the molar ratio of peptide antigen conjugate to the D2 is between about 20:1 to 1:2, or about 10:1 to about 1:1 or about 4:1 to about 2:1.

In some embodiments of vaccines, wherein at least one A is a glycopeptide, e.g. A is a glycopeptide selected from HGVT*S*APDT*RPAPGS*T*APPA (SEQ ID NO:319), DT*RPAPGS*T*APPAHGVT*S*AP (SEQ ID NO:320), GS*T*APPAHGVT*S*APDT*RPAPGS*T*APPA (SEQID NO:321), GVT*S*APDT*RPAP (SEQ ID NO:322), APDT*RPAPGS*T*A (SEQ ID NO:323), GS*T*APPAHGVT*S*AP (SEQ ID NO:324), VT*S*AP (SEQ ID NO:540), DT*RPAP (SEQ ID NO:325) and GS*T*AP (SEQ ID NO:326), wherein * is an O-linked glycan and each occurrence is independently selected from sialyl lewis x, sialyl lewis a, lewis y, lewis x, Tn, sTn, TF, sTF.

In some embodiments, S is absent. In other embodiments, S is present.

In some embodiments of vaccines, the vaccine further comprises an amphiphile having the formula S-[B]-[U]-H,
wherein S is a solubilizing block;
B is a spacer;
H is a hydrophobic block;
U is a linker;
[ ] denotes that the group is optional; and
— denotes that the two adjacent groups are directly attached to one another by a covalent bond or indirectly to one another via a suitable linker X,
wherein the S of the amphiphile comprises a dendron amplifier.

In some embodiments of vaccines, the S of the amphiphile comprises two or more solubilizing groups (SGs) independently selected from amines, carboxylic acids or sugar molecules, wherein the sugar molecules are independently selected from mannose, sialyl lewis x, sialyl lewis a, lewis y, lewis x, Tn, sTn, TF, sTF, Globo H, SSEA-3, GM2, GD2, GD3 and Fucosyl GM1 and combinations thereof.

In some embodiments of vaccines, the at least one peptide antigen conjugate comprises at least one A selected from autoantigens, alloantigens, and allergens.

In some embodiments of vaccines, the vaccine further comprises at least one D selected from inhibitors of mTOR, RORγt, CDK8/19, and HDAC and agonists of AHR, RAR and A₂a.

In some embodiments of vaccines, the vaccine further comprises a second drug molecule (D2) independently selected from inhibitors of mTOR, RORγt, CDK8/19, and HDACs, agonists of AHR, RAR and A₂a, and immunostimulants selected from agonists of NLRs, CLRs, TLRs and STING, provided that D and D2 bind to different receptors.

In some embodiments of vaccines, the D2 is selected from agonists of NLRs, CLRs, TLRs and STING. In other embodiments, the D2 is selected from agonists of TLR-3, TLR-7, TLR-8, TLR-7/8, TLR-9 and STING. In other embodiments, the D2 is selected from RNA and imidazoquinoline agonists of TLR-7, TLR-8 and TLR-7/8.

In some embodiments of vaccines, the vaccine further comprises a third drug molecule (D3) independently selected from inhibitors of mTOR, RORγt, CDK8/19, and HDACs, agonists of AHR, RAR and A₂a, and immunostimulants selected from agonists of NLRs, CLRs, TLRs and STING, provided that D, D2 and D3 bind to different receptors.

In some embodiments of vaccines, the at least one D is selected from AZD-8055, AZD2016, KU-0063794, CC223, Torin-1, Torin-2, INK-128, WYE354, WYE132, OSI-027, OXA-01, PI-103, NVP-BEZ235, GNE-493, GSK2126458, rapamycin, tacrolimus, everolimus, RAD001, CCI-779 and AP23573.

In some embodiments of vaccines, the molar ratio of total peptide antigen conjugate to the at least one D is between about 20:1 to 1:2, or about 10:1 to about 1:1 or about 4:1 to about 2:1.

In some embodiments of vaccines, the vaccine further comprises an amphiphile having the formula S-[B]-[U]-H,
wherein S is a solubilizing block;
B is a spacer;
H is a hydrophobic block;
U is a linker;
[ ] denotes that the group is optional; and
— denotes that the two adjacent groups are directly attached to one another by a covalent bond or indirectly to one another via a suitable linker X,
wherein the S of the amphiphile comprises a dendron amplifier.

In some embodiments of vaccines, the S of the amphiphile comprises two or more solubilizing groups (SGs) independently selected from carboxylic acids, phosphoserine and sugar molecules, wherein the sugar molecules are independently selected from mannose, glucose, glucosamine, N-acetyl glucose, galactose, galactosamine, N-acetyl galactosamine, and agonists of CD22a.

In some embodiments of vaccines, S of at least one peptide antigen conjugate comprises SGs selected from amines. In other embodiments, the S of the peptide antigen conjugate comprises one or more lysine or ornithine residues.

In some embodiments of vaccines, the peptide antigen conjugate has a net positive charge between about +1 to about +10 at physiologic pH. In other embodiments, the peptide antigen conjugate has a net positive charge between about +2 to about +6 or between about +3 to about +5 at physiologic pH.

In some embodiments of vaccines, the amphiphile is present and the molar ratio of peptide antigen conjugate to amphiphile is between about 4:1 to 1:20.

In some embodiments of vaccines, the amphiphile comprise carboxylic acids and has net negative charge. In other embodiments, the amphiphile comprises carboxylic acids selected from beta alanine and succinic acid.

In some embodiments of vaccines, the average net charge of the at least one peptide antigen conjugate is positive at physiologic pH and the molar ratio of peptide antigen conjugate to amphiphile is between about 4:1 to about 2:1 or about 1:2 to about 1:16, or about 1:2 to about 1:4. In certain preferred embodiments, the molar ratio is about 1:1.

The present disclosure also relates to a vaccine comprising an expression system comprising DNA or RNA encoding for at least one peptide antigen (A), wherein the vaccine further comprises at least one drug molecule (D) selected from Treg promoting immunomodulators.

In some embodiments of the vaccines comprising an expression system, the at least one D is selected from ATP-competitive mTOR inhibitors.

In some embodiments of vaccines comprising an expression system, D is selected from AZD-8055, AZD2016, KU-0063794, CC223, Torin-1, Torin-2, INK-128, WYE354, WYE132, OSI-027, OXA-01, PI-103, NVP-BEZ235, GNE-493, GSK2126458, rapamycin, tacrolimus, everolimus, RAD001, CCI-779 and AP23573.

In some embodiments of vaccines comprising an expression system, the peptide antigen (A) is selected from autoantigens, alloantigens and allergens.

In some embodiments of vaccines comprising an expression system, the vaccine further comprises a cationic liposomal particle.

In some embodiments of vaccines, the vaccine comprises particles further comprising an amphiphile and one or more peptide antigen conjugates. In preferred embodiments of vaccines, the vaccine comprises particles that comprise an amphiphile having the formula S-[B]-[U]-H and at least one peptide antigen conjugate having the formula [S]-[E1]-A-[E2]-[U]-H or H-[U]-[E1]-A-[E2]-[S], wherein A is a peptide antigen, S is a solubilizing block; E1 and E2 are N- and C-terminal extensions, respectively; B is a spacer; U is a linker molecule; H is hydrophobic block; [ ] denotes that the groups is optional; — denotes that the two adjacent groups are directly attached to one another by a covalent bond or indirectly to one another via a suitable linker X; and S, U and H of the amphiphile and the peptide antigen conjugate may be the same, different, or comprise one or more of the same functional groups or moieties.

In some embodiments of vaccines, the amphiphiles and/or peptide antigen conjugates further comprise one or more drug molecules (D). The drug molecules (D) may either be linked directly or indirectly via X1 to the hydrophobic block (H) of the amphiphile and/or peptide antigen conjugate (e.g., S-[B]-[U]-H-D and/or [S]-[E1]-A-[E2]-[U]-H-D). The drug molecule (D) may be admixed with the amphiphile and/or peptide antigen conjugate (e.g., D+S-[B]-[U]-H+[S]-[E1]-A-[E2]-[U]-H) or the drug molecule (D) may be in form of a drug molecule conjugate (i.e. D-[U]-H or H-D) that is admixed with the amphiphile and/or peptide antigen conjugate (e.g., D-H+S-[B]-[U]-H+[S]-[E1]-A-[E2]-[U]-H). Preferred compositions of vaccines further comprising drug molecules (D) are described throughout the specification. The D is bonded directly or indirectly as a side chain or as part of a side chain group to the adjacent group.

In preferred embodiments of vaccines, the vaccine comprises particles comprising amphiphiles and one or more peptide antigen conjugates, which further comprises a drug molecule (D) selected from immunomodulators. The drug molecule (D) selected from immunomodulators may either be linked directly or indirectly via X1 to the hydrophobic block (H) of the amphiphile and/or peptide antigen conjugate (e.g., S-[B]-[U]-H-D and/or [S]-[E1]-A-[E2]-[U]-HD); the drug molecule (D) may be admixed with the amphiphile and peptide antigen conjugate (e.g., D+S-[B]-[U]-H+[S]-[E1]-A-[E2]-[U]-H); or, the drug molecule (D) may be in the form of a drug molecule conjugate (i.e. D-[U]-H or H-D) that is admixed with the amphiphile and peptide antigen conjugate (e.g., D-H+S-[B]-[U]-H+[S]-[E1]-A-[E2]-[U]-H). Preferred compositions of vaccines further comprising drug molecules (D) are described throughout the specification.

In some embodiments of vaccines for treating or preventing autoimmune diseases, the peptide antigen conjugate comprises an antigen (A) selected from a self-antigen (sometimes referred to as an autoantigen). In some embodiments of vaccines for treating or preventing allergies, the peptide antigen conjugate comprises an antigen (A) selected from an allergen. In some embodiments of vaccines for treating or preventing cancer, the peptide antigen conjugate comprises an antigen (A) selected from self-antigens, neoantigens or viral antigens. In some embodiments of vaccines for treating or infectious diseases, the peptide antigen conjugate comprises an antigen (A) selected from viruses, bacteria, protozoa or fungi. In still other embodiments, the vaccine comprises an antigen selected from an endogenously produced protein and the vaccine is used for the treatment of cardiovascular disease. In still other embodiments of vaccines, the antigen is selected from small molecule haptens and the vaccine is used to prevent toxicity upon exposure to chemical toxins, including nerve agents. Preferred antigens as well as preferred methods for selecting antigens for treating different diseases are described throughout the specification.

It was found that particles comprising certain compositions of amphiphiles had particular utility for delivery of small molecule drugs for various applications, including treatment of cancer, inflammation, autoimmune diseases, macular degeneration as well as diseases of vital organs, including the CNS, heart and liver, and metabolic diseases.

In some embodiments of compositions for cancer treatment, the cancer treatment comprises particles comprising amphiphiles and drug molecules selected from chemotherapeutics and/or immunomodulators. In preferred embodiments of cancer treatments, the particle comprises amphiphiles having the formula S-[B]-[U]-H and a drug, D, wherein S is a solubilizing block; B is a spacer; U is a linker molecule; H is a hydrophobic block; [ ] denotes that the groups is optional; and, the drug, D, is associated with the particles through covalent or non-covalent interactions.

The present disclosure also relates to a peptide antigen conjugate having the formula selected from S-[E1]-A-[E2]-[U]-H-[D] and [D]-H-[U]-[E1]-A-[E2]-S or a peptide antigen fragment having the formula selected from S-[E1]-A-[E2]-[U1] and [U1]-[E1]-A-[E2]-S.

In some embodiments, the drug molecule (D) is linked to the hydrophobic block (H) of the amphiphile, e.g., S-[B]-[U]-H-D, wherein one or more D are bonded directly or indirectly via X1 at the end(s) or as part of a side chain group to the adjacent group. In other embodiments, the drug molecule is admixed with the amphiphile (e.g., D+S-[B]-[U]-H) or linked to a hydrophobic block (H) and admixed with the amphiphile (e.g., D-[B]-[U]-H+S-[B]-[U]-H, or H-D+S-[B]-[U]-H) and the drug is incorporated within the particles formed by the amphiphile. Preferred compositions of cancer treatments comprising amphiphiles and at least one chemotherapeutic and/or immunostimulant are described throughout the specification.

The present disclosure also relates to a peptide antigen conjugate having the formula selected from S-[E1]-A-[E2]-[U]-H-[D] and [D]-H-[U]-[E1]-A-[E2]-S or a peptide antigen fragment having the formula selected from S-[E1]-A-[E2]-[U1] and [U1]-[E1]-A-[E2]-S
wherein S is a solubilizing block;
H is a hydrophobic block,
   wherein one or more drug molecules (D) are optionally attached to each H directly or via a suitable linker X1;
A is a peptide antigen;
E1 is an N-terminal extension;
E2 is a C-terminal extension;
U is a linker;
U1 is a linker precursor;
[ ] denotes that the group is optional; and
— denotes that the two adjacent groups are directly attached to one another by a covalent bond or indirectly to one another via a suitable linker X, and
wherein S comprises one or more amino acids.

In some embodiments, the S comprises 2 to 12 amino acids. In other embodiments, S comprises 2 to 8 amino acids. In other embodiments, S comprises 4 to 6 amino acids. In other embodiments, S comprises 2, 3, 4, 5, 6, 7, 8, 9, 10, 11, or 12 amino acids.

In some embodiments, the S amino acids are selected from lysine, arginine and ornithine.

In some embodiments, the peptide antigen fragment has the formula A-[E2]-S.

In some embodiments, E1 and/or E2 are present and selected from cathepsin cleavable tetrapeptides of the formula P4-P3-P2-P1.

In some embodiments, E1 and/or E2 are present and selected from Ser-Pro-Val-Arg, Ser-Pro-Val-Cit and Ser-Pro-Val-aBut.

In some embodiments, the peptide antigen (A) comprise at least one amino acid selected from norleucine and alpha-aminobutyric acid.

In some embodiments, a vaccine comprising the peptide antigen conjugate or peptide antigen fragment disclosed herein.

In some embodiments, a method of activating, priming and/or expanding T cells, comprising adding an aqueous solution comprising the peptide antigen conjugate or peptide antigen fragment disclosed herein to the T cells in vitro or ex vivo.

In some embodiments, at least one of E1 and E2 is present in the peptide antigen conjugate or peptide antigen fragment.

In some embodiments, each E1 and/or E2, independently, comprises heptad repeats of formula (AAₐ-AA_{b}-AA_{c}-AA_{d}-AAₑ-AA_{f}-AA_{g})ₑ, wherein each occurrence of AAₐ and AA_{d} is independently selected from leucine, isoleucine, norleucine, valine, norvaline, T-leucine, allo-isoleucine, N-propyl glycine, methionine, and O-methyl serine;
each occurrence of AA_{b}, AA_{c} and AA_{f} is independently selected from alanine, cysteine, aspartic acid, glycine, asparagine, proline, serine, threonine, valine, alpha-amino-butyric acid, and norvaline; and each occurrence of AAₑ and AA_{g} is independently selected from charged amino acids, including but not limited to aspartic acid, glutamic acid, lysine, arginine, ornithine, sulfo-serine, and phosphoserine; and e is an integer selected from 1 to 6.

In some embodiments, each occurrence of AAₐ and AA_{d} is independently selected from leucine, isoleucine and norleucine.

In some embodiments, each occurrence of AA_{b}, AA_{c} and AA_{f} is independently selected from alanine, proline and serine.

In some embodiments, each occurrence of AAₑ and AA_{g} is independently selected from aspartic acid, glutamic acid, lysine, arginine, and ornithine.

In some embodiments, each E1 and/or E2, independently, comprise heptad repeats selected from (I-A-A-L-E-S-K)ₑ (SEQ ID NO:327), (I-A-A-L-K-S-K)ₑ (SEQ ID NO:328), (I-A-A-L-E-S-E)ₑ (SEQ ID NO:329), (I-A-A-L-K-S-E)ₑ (SEQ ID NO:330), (V-A-A-L-K-A-E)ₑ (SEQ ID NO:331), (I-A-A-L-K-A-E)ₑ (SEQ ID NO:332), (L-A-A-L-K-A-E)ₑ (SEQ ID NO:333), (V-S-A-L-K-A-E)ₑ (SEQ ID NO:334), (I-S-A-L-K-A-E)ₑ (SEQ ID NO:335), (L-S-A-L-K-A-E)ₑ (SEQ ID NO:336), (V-A-S-L-K-A-E)ₑ (SEQ ID NO:337), (I-A-S-L-K-A-E)ₑ (SEQ ID NO:338), (L-A-S-L-K-A-E)ₑ (SEQ ID NO:339), (V-S-S-L-K-A-E)ₑ (SEQ ID NO:340), (I-S-S-L-K-A-E)ₑ (SEQ ID NO:341), (L-S-S-L-K-A-E)ₑ (SEQ ID NO:342), (V-A-A-L-K-S-E)ₑ (SEQ ID NO:343), (L-A-A-L-K-S-E)ₑ (SEQ ID NO:344), (V-S-A-L-K-S-E)ₑ (SEQ ID NO:345), (I-S-A-L-K-S-E)ₑ (SEQ ID NO:346), (L-S-A-L-K-S-E)ₑ (SEQ ID NO:347), (V-A-S-L-K-S-E)ₑ (SEQ ID NO:348), and (I-A-S-L-K-S-E)ₑ (SEQ ID NO:349).

In some embodiments, each E1 and/or E2, independently, comprise heptad repeats selected from (K-S-E-L-A-A-I)ₑ (SEQ ID NO:350), (K-S-K-L-A-A-I)ₑ (SEQ ID NO:351), (E-S-S-L-A-A-I)ₑ(SEQ ID NO:352), (E-S-K-L-A-A-I)ₑ (SEQ ID NO:353), (E-A-K-L-A-A-V)ₑ (SEQ ID NO:354), (E-A-K-L-A-A-I)ₑ (SEQ ID NO:355), (E-A-K-L-A-A-L)ₑ (SEQ ID NO:356), (E-A-K-L-A-S-V)ₑ (SEQ ID NO:357), (E-A-K-L-A-S-I)ₑ (SEQ ID NO:358), (E-A-K-L-A-S-L)ₑ (SEQ ID NO:359), (E-A-K-L-S-A-V)ₑ (SEQ ID NO:360), (E-A-K-L-S-A-I)ₑ (SEQ ID NO:361), (E-A-K-L-S-A-L)ₑ (SEQ ID NO:362), (E-A-K-L-S-S-V)ₑ (SEQ ID NO:363), (E-A-K-L-S-S-I)ₑ (SEQ ID NO:364), (E-A-K-L-S-S-L)ₑ (SEQ ID NO:365), (E-S-K-L-A-A-V)ₑ (SEQ ID NO:366), (E-S-K-L-A-A-I)ₑ (SEQ ID NO:367), (E-S-K-L-A-A-L)ₑ (SEQ ID NO:368), (E-S-K-L-A-S-V)ₑ (SEQ ID NO:369), (E-S-K-L-A-S-I)ₑ (SEQ ID NO:370), (E-S-K-L-A-S-L)ₑ (SEQ ID NO:371), (E-S-K-L-S-A-V)ₑ (SEQ ID NO:372), (E-S-K-L-S-A-I)ₑ (SEQ ID NO:373).

In some embodiments, e is an integer selected from 1 to 4. In other embodiments, e is an integer selected from 2 or 3.

In some embodiments, 6 amino acids of each heptad are D-amino acids. In other embodiments, 7 amino acids of each heptad are D-amino acids.

In some embodiments, at one least of E1 and E2 is present in the at least one peptide antigen conjugate.

In some embodiments, each E1 and/or E2, independently, comprises heptad repeats of formula (AAₐ-AA_{b}-AA_{c}-AA_{d}-AAₑ-AA_{f}-AA_{g})ₑ, wherein each occurrence of AAₐ and AA_{d} is independently selected from leucine, isoleucine, norleucine, valine, norvaline, T-leucine, allo-isoleucine, N-propyl glycine, methionine, and O-methyl serine;
each occurrence of AA_{b}, AA_{c} and AA_{f} is independently selected from alanine, cysteine, aspartic acid, glycine, asparagine, proline, serine, threonine, valine, alpha-amino-butyric acid, and norvaline; and each occurrence of AAₑ and AA_{g} is independently selected from charged amino acids, including but not limited to aspartic acid, glutamic acid, lysine, arginine, ornithine, sulfo-serine, and phosphoserine; and e is an integer selected from 1 to 6.

In some embodiments, each occurrence of AAₐ and AA_{d} is independently selected from leucine, isoleucine and norleucine.

In some embodiments, each occurrence of AA_{b}, AA_{c} and AA_{f} is independently alanine, proline and serine.

In some embodiments, each occurrence of AAₑ and AA_{g} is independently selected from aspartic acid, glutamic acid, lysine, arginine, and ornithine.

In some embodiments, each E1 and/or E2, independently, comprises heptad repeats selected from (I-A-A-L-E-S-K)ₑ (SEQ ID NO:327), (I-A-A-L-K-S-K)ₑ (SEQ ID NO:328), (I-A-A-L-E-S-E)ₑ (SEQ ID NO:329), (I-A-A-L-K-S-E)ₑ (SEQ ID NO:330), (V-A-A-L-K-A-E)ₑ (SEQ ID NO:331), (I-A-A-L-K-A-E)ₑ (SEQ ID NO:332), (L-A-A-L-K-A-E)ₑ (SEQ ID NO:333), (V-S-A-L-K-A-E)ₑ (SEQ ID NO:334), (I-S-A-L-K-A-E)ₑ (SEQ ID NO:335), (L-S-A-L-K-A-E)ₑ (SEQ ID NO:336), (V-A-S-L-K-A-E)ₑ (SEQ ID NO:337), (I-A-S-L-K-A-E)ₑ (SEQ ID NO:338), (L-A-S-L-K-A-E)ₑ (SEQ ID NO:339), (V-S-S-L-K-A-E)ₑ (SEQ ID NO:340), (I-S-S-L-K-A-E)ₑ (SEQ ID NO:341), (L-S-S-L-K-A-E)ₑ (SEQ ID NO:342), (V-A-A-L-K-S-E)ₑ (SEQ ID NO:343), (L-A-A-L-K-S-E)ₑ (SEQ ID N0344), (V-S-A-L-K-S-E)ₑ (SEQ ID NO:345), (I-S-A-L-K-S-E)ₑ (SEQ ID NO:346), (L-S-A-L-K-S-E)ₑ (SEQ ID NO:347), (V-A-S-L-K-S-E)ₑ (SEQ ID NO:348), and (I-A-S-L-K-S-E)ₑ (SEQ ID NO:349).

In some embodiments, each E1 and/or E2, independently, comprises heptad repeats selected from (K-S-E-L-A-A-I)ₑ (SEQ ID NO:350), (K-S-K-L-A-A-I)ₑ (SEQ ID NO:351), (E-S-S-L-A-A-I)ₑ(SEQ ID NO:352), (E-S-K-L-A-A-I)ₑ (SEQ ID NO:353), (E-A-K-L-A-A-V)ₑ (SEQ ID NO:354), (E-A-K-L-A-A-I)ₑ (SEQ ID NO:355), (E-A-K-L-A-A-L)ₑ (SEQ ID NO:356), (E-A-K-L-A-S-V)ₑ (SEQ ID NO:357), (E-A-K-L-A-S-I)ₑ (SEQ ID NO:358), (E-A-K-L-A-S-L)ₑ (SEQ ID NO:359), (E-A-K-L-S-A-V)ₑ (SEQ ID NO:360), (E-A-K-L-S-A-I)ₑ (SEQ ID NO:361), (E-A-K-L-S-A-L)ₑ (SEQ ID NO:362), (E-A-K-L-S-S-V)ₑ (SEQ ID NO:363), (E-A-K-L-S-S-I)ₑ, (SEQ ID NO:364) (E-A-K-L-S-S-L)ₑ (SEQ ID NO:365), (E-S-K-L-A-A-V)ₑ (SEQ ID NO:366), (E-S-K-L-A-A-I)ₑ (SEQ ID NO:367), (E-S-K-L-A-A-L)ₑ (SEQ ID NO:368), (E-S-K-L-A-S-V)ₑ (SEQ ID NO:369), (E-S-K-L-A-S-I)ₑ (SEQ ID NO:370), (E-S-K-L-A-S-L)ₑ (SEQ ID NO:371), (E-S-K-L-S-A-V)ₑ (SEQ ID NO:372), (E-S-K-L-S-A-I)ₑ (SEQ ID NO:373).

In some embodiments, e is an integer selected from 1 to 4. In other embodiments, e is an integer selected from 2 or 3.

In some embodiments, 6 amino acids of each heptad are D-amino acids. In other embodiments, 7 amino acids of each heptad are D-amino acids.

The present disclosure also relates to a method of inducing an immune response in a subject in need thereof, comprising administering to the subject at least one dose of a first vaccine (V1) followed by at least one dose of a second vaccine (V2), wherein V1 is a vaccine disclosed herein; and V2 is a viral vaccine.

In some embodiments, the T cell response in the subject is increased relative to the administration of only at least one dose of a first vaccine (V1).

In some embodiments, the T cell response in the subject is increased relative to the administration of only at least one dose of a second vaccine (V2).

In some embodiments, one dose of V1 is administered at a first time (V1T1). In other embodiments, two doses of V1 are administered at a first time (V1T1) and a second time (V1T2). In other embodiments, three doses of V1 are administered at a first time (V1T1), a second time (V1T2), and a third time (V1T3).

In some embodiments, one dose of V2 is administered at a first time (V2T1). In other embodiments, two doses of V2 are administered at a first time (V2T1) and a second time (V2T2). In other embodiments, three doses of V2 are administered at a first time (V2T1), a second time (V2T2), and a third time (V2T3).

In some embodiments, V1 is administered by intramuscular or intravenous route.

In some embodiments, V2 is administered by intravenous route.

In some embodiments, the initial dose of V2 is administered from 1 to 6 weeks following the final dose of V1. In other embodiments, the initial dose of V2 is administered from 1 to 12 weeks following the final dose of V1.

In some embodiments, V2 is an adenovirus vector vaccine.

In some embodiments, the adenovirus encodes for a peptide antigen (A) of V1.

In some embodiments, V2 is a ChAdOx vaccine.

### Linkers

The term linker refers to any molecule that joins together any two or more molecules (or “moieties”), such as any two or more components of amphiphiles, peptide antigen conjugates, hapten conjugates or drug conjugates, and may additionally perform any one or more of the following functions: I) increase or decrease water solubility; II) increase distance between any two components; III) impart rigidity or flexibility; or, IV) modulate the rate of degradation of the link between any two or more different molecules. As used herein, the term “linker” may be used to describe linkers (U), suitable linkers (X), such as X1, X2, X3, X4 and X5, and extensions (E1 or E2).

Linkers that have particular utility are named, and specific, preferred compositions of those named linkers are described throughout the specification. Accordingly, extensions E1 and E2 are optional peptide-based linkers extending from the N- and C-termini of the peptide antigen (A), respectively, which may be included between the solubilizing block (S) and the antigen (A) or between the antigen (A) and hydrophobic block (H) or between the antigen (A) and optional Linker U. The spacer (B) is a linker between the solubilizing block (S) and the hydrophobic block (H) on amphiphiles. The molecule that results from the reaction of Linker precursor 1 (“U1”) linked either directly or indirectly to the solubilizing block or a drug (D) via a spacer (B) with Linker precursor 2 (“U2”) on a hydrophobic block (H) is referred to as a Linker U. Suitable linker X refers to any linker suitable for linking two or more adjacent groups. Suitable linkers preferred for joining drug molecules (D) to hydrophobic blocks (H) are referred to as X1. Suitable linkers preferred for joining aryl or heteroaryl groups to the hydrophobic block are referred to as X2. Suitable linkers used to join reactive functional groups (“FG4”) to the pharmacophore of drug molecules (D) are referred to as X3. Suitable linkers preferred for joining charged groups to hydrophobic block (H) are referred to as X4. Suitable linkers preferred for joining SG to S are referred to as X5.

The linker may use covalent or non-covalent means to join any two or more components. In preferred embodiments, a linker may join, i.e., link, any two components through a covalent bond. Covalent bonds are the preferred linkages used to join any two components and ensure that no component is able to immediately disperse from the other components following administration to a subject.

There are many suitable linkers that are well known to those of skill in the art and include, but are not limited to, straight or branched-chain carbon linkers, heterocyclic carbon linkers, rigid aromatic linkers, flexible ethylene oxide linkers, peptide linkers, or a combination thereof, which, for covalent linkers, further comprise two or more functional groups, which may be the same or different, that are used to link any two molecules, e.g., any two components of amphiphiles, peptide antigen conjugates and/or drug conjugates, though covalent bonds.

In some embodiments, the carbon linker can include a C1-C18 alkane linker, e.g., a lower alkyl linker, such as C1-C6 (i.e., from one to six methylene units), which can serve to increase the space between two or more molecules, i.e., different components, while longer chain alkane linkers can be used to impart hydrophobic characteristics. Alternatively, hydrophilic linkers, such as ethylene oxide linkers, may be used in place of alkane linkers to increase the space between any two or more heterologous molecules and increase water solubility. In other embodiments, the linker can be a cyclic and/or aromatic compound, or poly(aromatic) compound that imparts rigidity. The linker molecule may comprise a hydrophilic or hydrophobic linker. In several embodiments, the linker includes a degradable peptide sequence that is cleavable by an intracellular enzyme (such as a cathepsin or the immunoproteasome).

For linking two components of amphiphiles, peptide antigen conjugates and drug conjugates, wherein at least one of the components comprises a peptide, it was found that linkers comprising between 2 and 7 methylene groups improved coupling of the two or components. In non-limiting examples, increasing the number of methylene units between the amide and the amine of the N-terminal amino acid of peptide-based hydrophobic blocks (H) led to improved coupling to other molecules, including U2, antigens (A), extension E2, spacers (B) and solubilizing blocks (S). Therefore, in preferred embodiments, the N-terminal amino acid of poly(amino acid)-based hydrophobic blocks (H) comprises two or more, typically between 2 and 7, such as 1, 2, 3, 4, 5, 6, 7 methylene units. For clarity, an amino acid with 2 methylene units is beta-alanine and an amino acid with 5 methylene units is amino-hexanoic acid. In certain preferred embodiments, the N-terminal amino acid of peptide-based hydrophobic blocks (H) is amino-hexanoic acid (sometimes referred to as Ahx; CAS number 60-32-3). In other embodiments, the N-terminal amino acid of peptide-based hydrophobic blocks (H) is beta-alanine.

In some embodiments, the linker may comprise poly(ethylene oxide) (PEG). The length of the linker depends on the purpose of the linker. For example, the length of the linker, such as a PEG linker, can be increased to separate any two or more components, for example, to reduce steric hindrance, or in the case of a hydrophilic PEG linker can be used to improve water solubility. The linker, such as PEG, may be between about 1 and about 24 monomers in length, such as 1, 2, 3, 4, 5, 6, 7, 8, 9, 10, 11, 12, 13, 14, 15, 16, 17, 18, 19, 20, 21, 22, 23, 24 monomers in length or more. When used as a spacer (B), the PEG may be up to 45 monomers in length or more, though, typically between 4 and 36 monomers in length.

In some embodiments, wherein the linker comprises a carbon chain, the linker may comprise a chain of between about 1 or 2 and about 18 carbons, such as 1, 2, 3, 4, 5, 6, 7, 8, 9, 10, 11, 12, 13, 14, 15, 16, 17, 18 carbons in length or more. In some embodiments, wherein the linker comprises a carbon chain, the linker may comprise a chain of between about 12 and about 20 carbons. In some embodiments, wherein the linker comprises a carbon chain, the linker may comprise a chain of between no more than 18 carbons, typically between about 1 and 6 carbon atoms.

The linkage used to join any two or more molecules, e.g., any two or more components of amphiphiles, peptide antigen conjugates and/or drug conjugates may comprise any suitable functional group, including but not limited to amides, esters, ethers, thioethers, silyl ethers, disulfides, carbamates, carbamides, hydrazides, hydrazones, acetals and triazoles.

In non-limiting examples of a covalent linkage, a click chemistry reaction may result in a triazole that links, i.e., joins together, any two components of the amphiphile, peptide antigen conjugate, or drug molecule conjugate. In several embodiments, the click chemistry reaction is a strain-promoted [3+2]azide-alkyne cyclo-addition reaction. An alkyne group and an azide group may be provided on respective molecules to be linked by “click chemistry”. In some embodiments, an antigen (A) bearing an azide functional group is coupled to a hydrophobic block (H) having an appropriate reactive group, such as an alkyne, for example, a dibenzylcyclooctyne (DBCO).

In some embodiments, an amine is provided on one molecule and may be linked to another molecule by reacting the amine with any suitable electrophilic group such as carboxylic acids, acid chlorides, activated esters (for example, NHS ester), which results in an amide bond; the amine may be reacted with alkenes (via Michael addition); the amine may be reacted with aldehydes and ketones (via Schiff base); or, the amine may be reacted with activated carbonates or carbamates to yield a carbamate.

In some embodiments, the linker is cleavable under intracellular conditions, such that cleavage of the linker results in the release of any component linked to the linker, for example, a drug molecule (D).

For example, the linker can be cleavable by enzymes localized in intracellular vesicles (for example, within a lysosome or endosome or caveolae) or by enzymes, in the cytosol, such as the proteasome, or immunoproteasome. The linker can be, for example, a peptide linker that is cleaved by protease enzymes, including, but not limited to proteases that are localized in intracellular vesicles, such as cathepsins in the lysosomal or endosomal compartments of cells.

The peptide linker is typically between 1-10 amino acids, such as 1, 2, 3, 4, 5, 6, 7, 8, 9, 10, or more (such as up to 20) amino acids long, such as 11, 12, 13, 14, 15, 16, 17, 18, 19, 20 or more amino acids long. When used as a spacer (B), the peptide linker may be up to about 45 amino acids. Certain dipeptides are known to be hydrolyzed by proteases that include cathepsins, such as cathepsins B and D and plasmin, (see, for example, Dubowchik and Walker, 1999, Pharm. Therapeutics 83:67-123). For example, a peptide linker that is cleavable by the thiol-dependent protease cathepsin-B, can be used (for example, a Phe-Leu or a Gly-Phe-Leu-Gly (SEQ ID NO:1) linker). Other examples of such linkers are described, for example, in U.S. Pat. No. 6,214,345, incorporated herein by reference. In certain such embodiments, the peptide linker cleavable by an intracellular protease is a Val-Cit linker or a Phe-Lys linker (see, for example, U.S. Pat. No. 6,214,345, which describes the synthesis of doxorubicin with the Val-Cit linker). Note: for examples of amino acids and peptides provided in throughout the specification (either within the text of figures), unless otherwise specified, it should be understood that the peptides and amino acids are L-amino acids.

The cleavable peptide linker can be selected to promote processing (i.e., hydrolysis) of the peptide linker following intracellular uptake by immune cells. The sequence of the cleavable peptide linker can be selected to promote processing by intracellular proteases, such as cathepsins in intracellular vesicles or the proteasome or immunoproteasome in the cytosolic space.

In several embodiments, linkers comprising peptide sequences of the formula Pn . . . P4-P3-P2-P1 are used to promote recognition by cathepsins, wherein P1 is selected from arginine, lysine, acetyl lysine (i.e., the epsilon amine is acetylated), boc protected lysine (i.e., the epsilon amine is boc protected), citrulline, glutamine, threonine, leucine, norleucine, alpha-aminobutyric acid (abbreviated as “a-But” herein) or methionine; P2 is selected from glycine, serine, leucine, valine or isoleucine; P3 is selected rom glycine, serine, alanine, proline, or leucine; and P4 is selected from glycine, serine, arginine, lysine, acetyl lysine (i.e., the epsilon amine is acetylated), boc protected lysine, aspartic acid, glutamic acid or beta-alanine. In non-limiting examples a tetrapeptide linker of the formula P4-P3-P2-P1 linked through an amide bond to another molecule and has the sequence Lys-Pro-Leu-Arg (SEQ ID NO:2). For clarity, the amino acid residues (Pn) are numbered from proximal to distal from the site of cleavage, which is C-terminal to the P1 residue, for example, the amide bond between P1-P1′ is hydrolyzed. Suitable peptide sequences that promote cleavage by endosomal and lysosomal proteases, such as cathepsin, are well described in the literature (see: Choe, et al., J. Biol. Chem., 281:12824-12832, 2006).

In several embodiments, linkers comprising peptide sequences are selected to promote recognition by the proteasome or immunoproteasome. Peptide sequences of the formula Pn . . . P4-P3-P2-P1 are selected to promote recognition by proteasome or immunoproteasome, wherein P1 is selected from basic residues and hydrophobic, branched residues, such as arginine, lysine, leucine, isoleucine and valine; P2, P3 and P4 are optionally selected from leucine, isoleucine, valine, lysine and tyrosine. In non-limiting examples, a cleavable linker of the formula P4-P3-P2-P1 that is recognized by the proteasome is linked through an amide bond at P1 to another molecule and has the sequence Tyr-Leu-Leu-Leu (SEQ ID NO:3). Sequences that promote degradation by the proteasome or immunoproteasome may be used alone or in combination with cathepsin cleavable linkers. In some embodiments, amino acids that promote immunoproteasome processing are linked to linkers that promote processing by endosomal proteases. A number of suitable sequences to promote cleavage by the immunoproteasome are well described in the literature (see: Kloetzel, et al., Nat. Rev. Mol. Cell Biol., 2:179-187), 2001, Huber, et al., Cell, 148:727-738, 2012, and Harris et al., Chem. Biol., 8:1131-1141, 2001).

In certain preferred embodiments, drug molecules (D) are linked to hydrophobic blocks (H) via linker X1 comprising an enzyme degradable peptide. A non-limiting example is shown here:

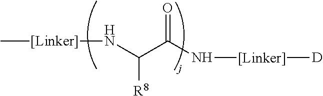

wherein D is a drug molecule; “Linker” is any suitable linker molecule; j denotes any integer, though, j is typically 1 to 6 amino acids, such as 1, 2, 3, 4, 5 or 6 amino acids; R⁸ is any suitable amino acid side group; the N-terminal amine of the peptide is linked either directly or via the ends, e.g., to the N- or C-termini of a hydrophobic block (H) comprising poly(amino acids), either directly or via U, or through reactive monomers comprising the hydrophobic block (H); and, brackets “[ ]” denote that the group is optional.

In certain preferred embodiments of drug molecules linked to hydrophobic blocks (H) via linker X1 comprising an enzyme degradable peptide, the drug molecule (D) is linked directly to the peptide through an amide bond as shown here:

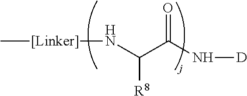

In non-limiting examples of the above structure, wherein the N-terminal Linker group is present and selected from beta alanine the structure is:

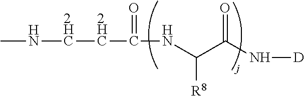

In some embodiments, the drug molecule (D) is linked to the peptide via a self-immolative carbamate linker. A non-limiting example is shown here:

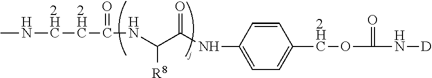

In the above example, wherein j is 4 and the amino acids are Serine-Lysine(Ac)—Valine-nor-Leucine, the structure is:

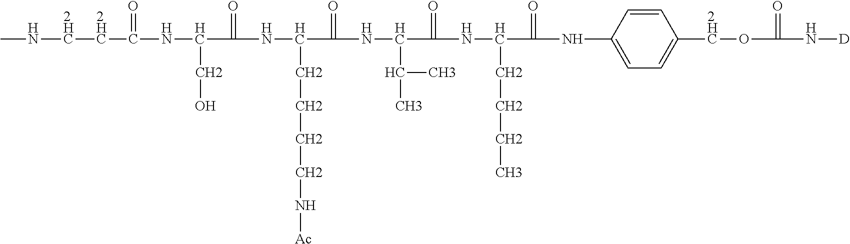

In some embodiments, drug molecules (D) are linked to hydrophobic blocks (H) through a sulfatase degradable linker X1, wherein hydrolysis of a sulfate by sulfatase results in release of the drug molecule from the linker. A number of arylsulfatase and alkysulfatase degradable linkers have recently been described (e.g., see: Bargh, et al., 2020, Chem. Sci. 11, 2375). In some embodiments of the present disclosure, drug molecules are linked to hydrophobic blocks (H) through sulfatase degradable linkers. Non-limiting examples are shown here for clarity:

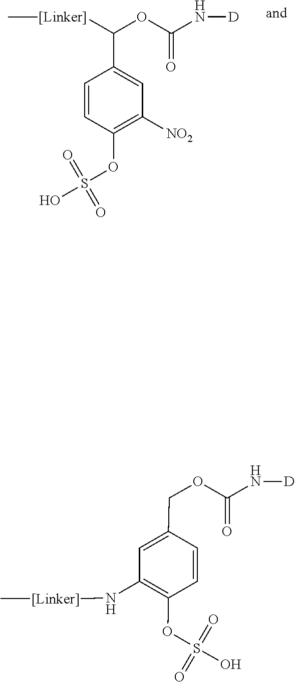

wherein D is a drug molecule; “Linker” is any suitable linker molecule linked either directly or via ends, e.g., to the N- or C-termini of a hydrophobic block (H) comprising poly(amino acids), either directly or via U, or through reactive monomers comprising the hydrophobic block (H); and, brackets “[ ]” denote that the group is optional.

Non-limiting examples of the above structures, wherein the “Linker” is present and selected from short alkyl linkers linked to the hydrophobic block through an amide are shown here for clarity:

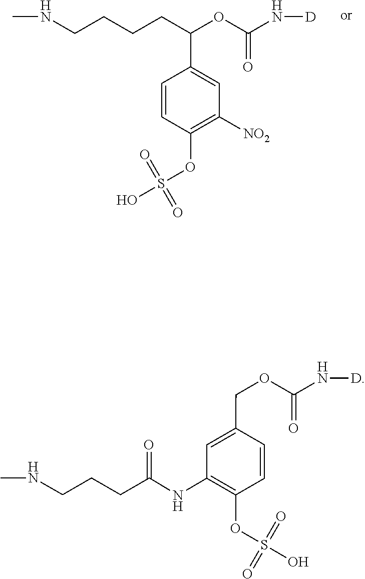

In other embodiments, any two or more components may be joined together through a pH-sensitive linker X that is sensitive to hydrolysis under acidic conditions. A number of pH-sensitive linkers are familiar to those skilled in the art and include for example, a hydrazone, carbohydrazone, semicarbazone, thiosemicarbazone, cis-aconitic amide, orthoester, acetal, ketal, silylether or the like (see, for example, U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661).

In certain embodiments, different components (e.g., drug molecule and hydrophobic block (H)) are linked together through pH-sensitive linkers that are stable at blood pH, e.g., at a pH of about 7.4, but undergo more rapid hydrolysis at endosomal/lysosomal pH, ˜pH 5-6.5. In certain, preferred embodiments, drug molecules (D) are linked to hydrophobic blocks (H) through reactive monomers via a pH-sensitive bonds, such as hydrazone bonds that result from the reaction between a ketone and a hydrazine. The functional group hydrazine linked to a carbonyl is sometimes referred to as hydrazide, though, hydrazine is meant to broadly refer to —NH—NH₂ groups, including when linked to carbonyl, e.g., C(O)—NH—NH₂. pH-sensitive linkages, such as a hydrazone, provide the advantage that the bond is stable at physiologic pH, at about pH 7.4, but is hydrolyzed at lower pH values, such as the pH of intracellular vesicles.

In certain preferred embodiments, drug molecules are linked by a linker X1 comprising a ketone and may be represented by the formula:

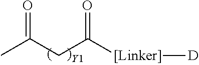

wherein D is any drug molecule; “Linker” is any suitable linker molecule; y1 denotes an integer between 1 to 6, preferably 4; brackets “[ ]” denote that the group is optional; and, wherein the ketone in the above example is used to link the linker linked drug molecule (D) to a reactive monomer through a hydrazone bond.

In the above example, wherein y1 is 4 and the drug molecule is linked directly (i.e., the “Linker” is absent) via an amide bond, the structure is:

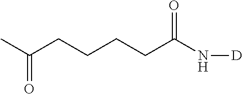

In preferred embodiments, drug molecules linked to ketones are linked to hydrophobic blocks (H) through hydrazone or carbohydrazone bonds. Non-limiting examples of drug molecules linked to a glutamic acid-based reactive monomer (N) through hydrazone and carbohydrazone bonds are shown here:

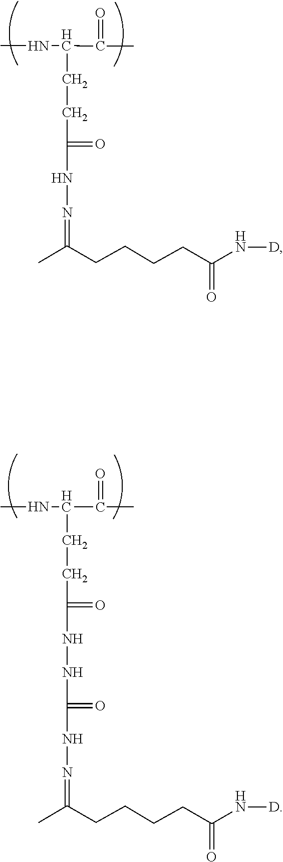

In some embodiments, the drug molecule comprises a ketone and may be linked directly to reactive monomers through hydrazone or carbohydrazone.

In other embodiments, the linker comprises a linkage that is cleavable under reducing conditions, such as a reducible disulfide bond. Many different linkers used to introduce disulfide linkages are known in the art (see, for example, Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987); Phillips et al., Cancer Res. 68:92809290, 2008). See also U.S. Pat. No. 4,880,935).

In preferred embodiments, the linker X1 linking a hydrophobic block (H) and one or more drug molecules (D) is a short alkyl or PEG linker. In other preferred embodiments, the linker X1 linking a hydrophobic block (H) and one or more drug molecules (D) is an enzyme degradable linker, such as a cathepsin degradable peptide or sulfatase degradable linker. In other preferred embodiments, the linker X1 linking a hydrophobic block (H) and one or more drug molecules (D) comprises an enzyme degradable peptide and a self-immolative linker.

X can be any suitable linker, though, in preferred embodiments, the linker X linking any two or more groups, is a short alkyl (i.e., lower alkyl) or PEG linker, e.g., a PEG linker with between about 1 to about 24 monomeric units.

### Extensions (E1 and E2)

The optional N- and C-terminal extensions (E1 and E2) denote moieties linked to the N- and C-terminus of the peptide antigen (A), respectively. The N- and C-terminal extensions E1 and E2 may comprise any one or more of the following: amino acids, including non-natural amino acids; hydrophilic ethylene oxide monomers (e.g., PEG); hydrophobic alkane chains; or the like; or combinations thereof. The N- and C-terminal extensions E1 and E2 are attached to the peptide antigen (A) through any suitable means, e.g., through amide bonds.

In some embodiments, the extensions (E1 and E2) function to control the rate of degradation of the peptide antigen (A) but may also perform any one or more additional functions. In some embodiments, the N- or C-terminal extension (E1 or E2) may be free (wherein one end of the N- or C-terminal extension is linked to the peptide antigen (A) and the other end is not linked to another molecule) and serve to slow degradation of the peptide antigen; for example, a E1 peptide-based extension may be linked to the N-terminus of the peptide antigen through an amide bond to slow degradation. In other embodiments, the N- and/or C-terminal extensions (E1 and/or E2) may be linked to a heterologous molecule and may function as a linker as well as to modulate peptide antigen (A) degradation. The N- and/or C-terminal extensions providing a linker function may link the peptide antigen either directly or indirectly through a Linker U to a hydrophobic block (H) and or solubilizing block (S). In some embodiments, the extensions (E1 and/or E2) function to provide distance, i.e., space, between any two heterologous molecules. In other embodiments, the extensions (E1 and/or E2) function to impart hydrophobic or hydrophilic properties to the peptide antigen conjugate. In still other embodiments, the composition of the extensions (E1 and/or E2) may be selected to impart rigidity or flexibility. In other embodiments, the N- and/or C-terminal extensions (E1 and/or E2) may help stabilize the particles formed by the peptide antigen conjugate.

In some embodiments, the extensions (E1 and/or E2) comprise charged functional groups, e.g., charged amino acid residues (e.g., arginine, ornithine, lysine, glutamic acid, aspartic acid, etc.), that impart charge at pH 7.4. The number of charged residues present in the extension can be used to modulate the net charge of the peptide antigen conjugate. Peptide-based extensions (E1 and/or E2) that are recognized by proteases and impart a particular electrostatic charge to stabilize particles formed by peptide antigen conjugates are described later.

Additionally, in some embodiments, C-terminal extensions (E2) added to peptide antigens (A) are selected to facilitate manufacturing of a peptides comprising the formula [S]-[E1]-A-E2-[U1], wherein [ ] denotes the group is optional. Accordingly, the amino acid sequence of peptide-based E2 can be selected to disrupt peptide $-sheet formation and prevent sequence truncation during solid-phase peptide synthesis. In non-limiting examples, a C-terminal di-peptide linker (E2), Gly-Ser, is incorporated during solid-phase peptide synthesis as a pseudoproline dipeptide (e.g., Gly-Ser(Psi(Me,Me)pro)). In additional embodiments, a proline is included in E2, e.g., Ser-Pro-Leu-Arg (SEQ ID NO:4); whereby the proline is included to both facilitate manufacturing and promote processing of the extension by endosomal proteases.

In some embodiments, the peptide antigen (A) is linked at the C-terminus to an E2 extension that is linked either directly or indirectly through a Linker (U) to a hydrophobic block, e.g., wherein the peptide antigen conjugate has the structure A-E2-U-H or A-E2-H. In some embodiments, an E1 extension is linked to the N-terminus of the peptide antigen (A) and an E2 extension is linked at the C-terminus of the peptide antigen (A), wherein either E1 or E2 are linked either directly or via a Linker (U) to a hydrophobic block (H), e.g. wherein the peptide antigen conjugate has the structure E1-A-E2-U-H, H-U-E1-A-E2, E1-A-E2-H, or H-E1-A-E2. In other embodiments, a peptide antigen (A) is linked at the N-terminus to an E1 extension that is linked either directly or via a Linker (U) to a hydrophobic block (H), e.g., wherein the peptide antigen conjugate has the structure H-U-E1-A or H-E1-A. In some embodiments, a solubilizing block is linked to an extension, E1 or E2, that is linked to the N- or C-terminus of the peptide antigen (A), respectively, wherein the extension that is not linked to the solubilizing block (S) is linked either directly or via a Linker (U) to the hydrophobic block (H), e.g., wherein the peptide antigen conjugate has the structure S-E1-A-E2-U-H, H-U-E1-A-E2-S, E1-A-E2-H, H-E1-A-E2-S.

In additional embodiments, solubilizing blocks (S) are linked to both E1 and E2 extensions that are linked to both the N- and C-termini of the peptide antigen (A), respectively; or, solubilizing blocks (S) are linked to the E1 extension linked to the N-terminus of the peptide antigen (A) but not to the E2 extension attached to the C-terminus of the peptide antigen (A), which may be linked either directly or through a Linker (U) to a hydrophobic block (H). A linker precursor U1 or Linker (U) may be linked to either of the extensions (E1 or E2) through any suitable means, such as an amide bond.

In preferred embodiments, the extensions (E1 and E2) are peptide sequences that are selected for recognition and hydrolysis by enzymes, such as proteases. The extensions (E1 and E2) are preferably cleavable peptides, including amino acids recognized by either or both endosomal proteases and/or the immunoproteasome.

In some embodiments, the N-terminal extension (E1) is a peptide sequence between about 1 to 8 amino acids in length, such as 1, 2, 3, 4, 5, 6, 7, or 8 amino acids, typically no more than 10 amino acids in length that is linked to the peptide antigen (A) through an amide bond formed between a carboxyl group of the E1 and the alpha amine of the N-terminal residue of the peptide antigen (A). The amide bond between E1 and the peptide antigen (A) may be cleaved by enzymes.

It is customary to number the amino acid positions in order of proximal to distal from the cleavage site, with amino acid positions C-terminal to the cleavage site indicated by the prime symbol (e.g., Pn′). For example, for a tetrapeptide extension (PN4-PN3-PN2-PN1) linked to the N-terminus of a peptide antigen (A) that is an octapeptide (PA1′-PA2′-PA3′-PA4′-PA5′-PA6′-PA7′-PA8′), e.g., PN4-PN3-PN2-PN1-PA1′-PA2′-PA3′-PA4′-PA5′-PA6′-PA7′-PA8′, the amide bond between PN1-PA1′ is recognized and hydrolyzed by an enzyme.

In some embodiments, the N-terminal extension (E1) is an enzyme degradable tetrapeptide that is recognized by endosomal proteases, wherein the PN1 position of a tetrapeptide extension (e.g., PN4-PN3-PN2-PN1) is preferably selected from arginine, lysine, citrulline, glutamine, threonine, leucine, norleucine, or methionine, for example, PN4-PN3-PN2-Arg; PN2 is selected from glycine, valine, leucine or isoleucine; PN3 is selected from glycine, serine, alanine, proline or leucine; and, PN4 is selected from glycine, serine, arginine, lysine, aspartic acid or glutamic acid. In some embodiments, the N-terminal extension (E1) is an enzyme degradable tripeptide that is recognized by endosomal proteases, wherein the PN1 position of a tripeptide extension (e.g., PN3-PN2-PN1) is preferably selected from arginine, lysine, citrulline, glutamine, threonine, leucine, norleucine, or methionine; PN2 is selected from glycine, valine, leucine or isoleucine; and PN3 is selected from glycine, serine, alanine, proline or leucine. In some embodiments, the N-terminal extension (E1) is an enzyme degradable di-peptide that is recognized by endosomal proteases, wherein the PN1 position of a dipeptide extension (e.g., PN2-PN1) is preferably selected from arginine, lysine, citrulline, glutamine, threonine, leucine, norleucine, or methionine; and PN2 is selected from glycine, valine, leucine or isoleucine. In still additional embodiments, the N-terminal extension (E1) is an amino acid that is recognized by endosomal proteases, wherein the PN1 position is preferably selected from arginine, lysine, citrulline, glutamine, threonine, leucine, norleucine, or methionine.

In other embodiments, the N-terminal extension (E1) is an enzyme degradable peptide that is recognized by the immunoproteasome, wherein the P1 position of a tetrapeptide extension (PN4-PN3-PN2-PN1) is preferably selected from isoleucine, leucine, norleucine or valine, for example, PN4-PN3-PN2-Leu.

In additional embodiments, the N-terminal extension (E1) is an enzyme degradable peptide that is recognized by both endosomal proteases and the immunoproteasome, wherein the PN5 and PN1 positions of an octapeptide extension (PN8-PN7-PN6-PN5-PN4-PN3-PN2-PN1) are selected from arginine, lysine, citrulline, glutamine, threonine, leucine, norleucine, or methionine for the PN5 position recognized by cathepsins, and isoleucine, leucine, norleucine or valine for the PN1 position recognized by the immuno-proteasome; for example, PN8-PN7-PN6-Arg-PN4-PN3-PN2-Leu. A non-limiting example of an N-terminal extension (E1) recognized by cathepsins and the immuno-proteasome is Lys-Pro-Leu-Arg-Tyr-Leu-Leu-Leu (SEQ ID NO:5).

Non-limiting examples of tetrapeptide N-terminal extensions (E1) that are recognized by the immunoproteasome include: Ser-Leu-Val-Cit (SEQ ID NO:6), Ser-Leu-Val-Leu (SEQ ID NO:7), Ser-Pro-Val-Cit (SEQ ID NO:8), Glu-Leu-Val-Arg (SEQ ID NO:9), Ser-Pro-Val-Arg (SEQ ID NO:10), Ser-Leu-Val-Arg (SEQ ID NO:11), Lys-Pro-Leu-Arg (SEQ ID NO:2), Lys-Pro-Val-Arg (SEQ ID NO:12), Glu-Leu-Val-Cit (SEQ ID NO:13), Glu-Leu-Val-Leu (SEQ ID NO:14), Glu-Pro-Val-Cit (SEQ ID NO:15), and Lys-Pro-Val-Cit (SEQ ID NO:16). Non-limiting examples of tripeptide N-terminal extensions (E1) include: Leu-Val-Cit, Leu-Val-Leu, Pro-Val-Cit, Leu-Val-Arg, Pro-Val-Arg, Pro-Leu-Arg, Gly-Val-Ser. Non-limiting examples of di-peptide N-terminal extensions (E1) include: Val-Cit, Val-Leu, Val-Arg, Leu-Arg. Non-limiting examples of single amino acid N-terminal extensions (E1) include Cit, Arg, Leu or Lys. In the above examples, Arg can be replaced with Lys; Lys can be replaced with Arg; Glu can be replaced with Asp; and Asp can be replaced with Glu. Note that Cit=citrulline.

In some embodiments, the E2 is a degradable peptide linked to the C-terminal residue of the peptide antigen (A) and comprises amino acid sequences that are recognized and hydrolyzed by certain proteases. In some embodiments, the C-terminal extension (E2) is a peptide sequence between about 1 to 8 amino acids in length, such as 1, 2, 3, 4, 5, 6, 7, or 8 amino acids, typically no more than 10 amino acids. In preferred embodiments, the C-terminal extension (E2) is linked to the peptide antigen (A) via an amide bond formed between the C-terminal carboxyl group of the peptide antigen (A) and the alpha amine of the N-terminal residue of the extension (E2). The amide bond between E2 and the peptide antigen (A) may be cleaved by enzymes. Note: that it is customary to number the amino acid positions in order of proximal to distal from the cleavage site, with amino acid positions C-terminal to the cleavage site indicated by the prime symbol (e.g., Pn′). For example, for a tetrapeptide extension (PC1′-PC2′-PC3′-PC4′) linked to the C-terminus of an octapeptide antigen (PA8-PA7-PA6-PA5-PA4-PA3-PA2-PA1), e.g., PA8-PA7-PA6-PA5-PA4-PA3-PA2-PA1-PC1′-PC2′-PC3′-PC4′, the amide bond between PA1-PC1′ is recognized and hydrolyzed by an enzyme.

In preferred embodiments of C-terminal extensions (E2), the C-terminal extension (E2) comprises amino acid sequences that are selected to promote immunoproteasome recognition and cleavage and optionally endosomal protease recognition. As peptide antigens (A) typically contain a C-terminal residue, for example, leucine, that promotes hydrolysis by the immunoproteasome, e.g., at the amide bond proximal to the C-terminal residue of the peptide antigen (A), extensions linked to the C-terminus of the peptide antigen (A) should be selected to promote immuno-proteasome recognition and cleavage at the amide bond proximal to the C-terminus of the peptide antigen (A). The immuno-proteasome favors small, non-charged amino acids at the PC1′ position adjacent to the C-terminal amino acid, PA1, of the peptide antigen (A), e.g., the amide bond between PA1-PC1′. However, endosomal proteases favor bulky hydrophobic amino acids (e.g., leucine, norleucine, methionine or glutamine) and basic amino acids (i.e., arginine and lysine). Therefore, C-terminal extensions may be selected to promote recognition by either or both classes of proteases.

In some embodiments, a peptide antigen (A) with the sequence PA8-PA7-PA6-PA5-PA4-PA3-PA2-PA1 is linked to a C-terminal peptide extension (E2) with the sequence PC1′ . . . PCn′, wherein n is an integer value from 1 to 8, for example, PA8-PA7-PA6-PA4-PA3-PA2-PA1-PC1′ . . . PCn′. The composition of the C-terminal extension (E2) depends on the length of the extension sequence used. In some embodiments, the C-terminal extension, E2, is a single amino acid PC1′ selected from Gly, Ala, Ser, Arg, Lys, Cit, Gln, Thr, Leu, Nle or Met. In additional embodiments, the C-terminal extension, E2, is a dipeptide, PC1′-PC2′, wherein PC1′ is selected from Gly, Ala or Ser; and PC2′ is selected from Gly, Ala, Ser, Pro, Arg, Lys, Cit, Gln, Thr, Leu, Nle, or Met. In additional embodiments, the C-terminal extension, E2, is a tripeptide, PC1′-PC2′-PC3′, wherein P1′ is selected from Gly, Ala, or Ser; PC2′ is selected from Gly, Ala, Ser, or Pro; and PC3′ is selected from Gly, Ser, Arg, Lys, Cit, Gln, Thr, Leu, Nle or Met.

In additional embodiments, the C-terminal extension, E2, is a tetrapeptide extension, PC1′-PC2′-PC3′-PC4′, wherein PC1′ is selected from glycine, alanine or serine; PC2′ is selected from glycine, alanine, serine, proline or leucine; PC3′ is selected from glycine, alanine, serine, valine, leucine or isoleucine; and PC4′ is selected from arginine, lysine, citrulline, glutamine, threonine, leucine, norleucine or methionine. In additional embodiments, the C-terminal extension, E2, is a pentapeptide, PC1′-PC2′-PC3′-PC4′-PC5′, wherein PC1′ is selected from glycine, alanine or serine; PC2′ is selected glycine, alanine, serine, proline, arginine, lysine, glutamic acid or aspartic acid; PC3′ is selected from glycine, alanine, serine, proline or leucine; PC4′ is selected from glycine, alanine, valine, leucine or isoleucine; and PC5′ is selected from arginine, lysine, citrulline, glutamine, threonine, leucine, norleucine or methionine. In additional embodiments, the C-terminal extension, E2, is a hexapeptide, PC1′-PC2′-PC3′-PC4′-PC5′-PC6′, wherein PC1′ is selected from glycine, alanine or serine; PC2′ is selected from glycine, alanine, serine or proline; PC3′ is selected from glycine, serine, proline, arginine, lysine, glutamic acid or aspartic acid; PC4′ is selected from proline or leucine; PC5′ is selected from glycine, alanine, valine, leucine or isoleucine; and PC6′ is selected from arginine, lysine, citrulline, glutamine, threonine, leucine, norleucine or methionine.

Non-limiting examples of hexapeptide C-terminal extensions (E2) include Gly-Gly-Lys-Leu-Val-Arg (SEQ ID NO:17), Gly-Gly-Lys-Pro-Leu-Arg (SEQ ID NO:18), Gly-Gly-Ser-Leu-Val-Arg (SEQ ID NO:19), Gly-Gly-Ser-Leu-Val-Cit (SEQ ID NO:20), Gly-Gly-Ser-Pro-Val-Cit (SEQ ID NO:21), Gly-Gly-Ser-Leu-Val-Leu (SEQ ID NO:22), Gly-Gly-Glu-Leu-Val-Arg (SEQ ID NO:23), Gly-Gly-Glu-Leu-Val-Leu (SEQ ID NO:24).

Non-limiting examples of pentapeptide C-terminal extensions (E2) include Gly-Ser-Leu-Val-Arg (SEQ ID NO:25), Gly-Ser-Leu-Val-Cit (SEQ ID NO:26), Gly-Lys-Pro-Val-Cit (SEQ ID NO:27), Gly-Lys-Pro-Val-Arg (SEQ ID NO:28), Gly-Ser-Leu-Val-Leu (SEQ ID NO:29), Gly-Glu-Leu-Val-Leu (SEQ ID NO:30).

Non-limiting examples of tetrapeptide C-terminal extensions (E2) include Ser-Leu-Val-Cit(SEQ ID NO:6), Ser-Leu-Val-Leu (SEQ ID NO:7), Ser-Pro-Val-Cit (SEQ ID NO:8), Glu-Leu-Val-Arg (SEQ ID NO:9), Ser-Pro-Val-Arg (SEQ ID NO:10), Ser-Leu-Val-Arg (SEQ ID NO:11), Lys-Pro-Leu-Arg (SEQ ID NO:2), Glu-Leu-Val-Cit (SEQ ID NO:13), Glu-Leu-Val-Leu (SEQ ID NO:14), Glu-Pro-Val-Cit (SEQ ID NO:15), Glu-Gly-Val-Cit (SEQ ID NO:31).

Non-limiting examples of tripeptide C-terminal extensions (E2) include Gly-Ser-Gly, Gly-Ser-Arg, Gly-Ser-Leu, Gly-Ser-Cit, Gly-Pro-Gly, Gly-Pro-Arg, Gly-Pro-Leu, Gly-Pro-Cit. Non-limiting examples of di-peptide C-terminal extensions (E2) include Gly-Ser, Gly-Pro, Val-Cit, Gly-Arg Gly-Cit. Non-limiting examples of single amino acid C-terminal extensions (E2) include Gly, Ser, Ala, Arg, Lys, Cit, Val, Leu, Met, Thr, Gln or Nle. In the above examples, Arg can be replaced with Lys; Lys can be replaced with Arg; Glu can be replaced with Asp; and Asp can be replaced with Glu.

The C-terminal linker (E2) linked to the C-terminus of the peptide antigen (A) may be selected for recognition (i.e., hydrolysis) by both the immunoproteasome and endosomal proteases. In non-limiting examples, a peptide antigen (A) with the sequence PA8-PA7-PA6-PA5-PA4-PA3-PA2-PA1 is linked at the C-terminus to a C-terminal tetrapeptide extension (E2) with the sequence PC1′-PC2′-PC3′-PC4′, wherein PC1′ is selected from glycine, alanine or serine and PC4′ is selected from arginine, lysine, citrulline, glutamine, threonine, leucine, norleucine, or methionine, for example, Ser-P3-P2-Arg. In some embodiments, an antigen with the sequence PA8-PA7-PA6-PA5-PA4-PA3-PA2-PA1 is linked at the C-terminus to a C-terminal hexapeptide extension (E2) with the sequence PC1′-PC2′-PC3′-PC4′-PC5′-PC6′, wherein PC1′ and PC2′ are selected from glycine, alanine, proline or serine and PC6′ is selected from arginine, lysine, citrulline, glutamine, threonine, leucine, norleucine, or methionine, for example, Gly-Gly-PC3′-PC4′-PC5′-Arg. A non-limiting example of a C-terminal extension (E2) that promotes processing by both the immuno-proteasome and cathepsins that is linked to the C-terminus of the peptide antigen (A) is Gly-Gly-Lys-Pro-Leu-Arg (SEQ ID NO:18). An additional non-limiting example of a C-terminal extension (E2) that is linked at the C-terminus of a peptide antigen (A) that favors processing by the immunoproteasome and cathepsins is Gly-Gly-Ser-Leu-Val-Cit (SEQ ID NO:20) or Gly-Gly-Ser-Pro-Val-Cit (SEQ ID NO:21).

In some embodiments of vaccines, the extension(s) (E1 and/or E2) comprise heptad repeats of formula (AA_{H}-AA_{P}-AA_{P}-AA_{H}-AA_{P}-AA_{P}-AA_{P})ₑ, wherein AA_{H} is typically a hydrophobic amino acid suitable for a coil domain, AA_{P} is typically a hydrophilic or small amino acid suitable for a coil domain and e denotes an integer typically selected from between 1 and 6, such as 1, 2, 3, 4, 5 and 6, preferably between 1 and 4, most preferably 2 or 3. Each amino acid of heptad repeats of formula (AA_{H}-AA_{P}-AA_{P}-AA_{H}-AA_{P}-AA_{P}-AA_{P})ₑ should be carefully selected to ensure that resulting sequences form a helical secondary structure that can form a coiled coil superstructure. To help guide selection, heptad repeats of formula AA_{H}-AA_{P}-AA_{P}-AA_{H}-AA_{P}-AA_{P}-AA_{P})ₑ may be further delineated according to the following formula, T wherein AAₐ and AA_{d} are typically selected from hydrophobic amino acids; AA_{b}, AA_{c} and AA_{f} are typically selected from small, amino acids; and, AAₑ and AA_{g} are typically selected from charged amino acids. For clarity, amino acids refers to both proteinogenic and non-proteinogenic amino acids.

For purposes of selecting amino acids for use in heptad repeats described herein, general properties of proteinogenic amino acids, also referred to as natural amino acids as well as non-limiting examples of non-proteinogenic amino acids, also known as non-natural amino acids, are summarized in the table below.

| | | Ali- | Aro- | Hydro- | |
|---|---|---|---|---|---|
| Amino Acid | Small | phatic | matic | phobic | Charged |
| Alanine (A, Ala) | T | T | F | T | F |
| Cysteine (C, Cys) | T | F | F | T | F |
| Aspartic Acid (D, Asp) | T | F | F | F | T |
| Glutamic Acid (E, Glu) | F | F | F | F | T |
| Phenylalanine (F, Phe) | F | F | T | T | F |
| Glycine (G, Gly) | T | F | F | T | F |
| Histidine (H, His) | F | F | T | F | T |
| Isoleucine (I, Ile) | F | T | F | T | F |
| Lysine (K, Lys) | F | F | F | F | T |
| Leucine (L, Leu) | F | F | F | T | F |
| Methionine (M, Met) | F | F | F | T | F |
| Asparagine (N, Asn) | T | F | F | F | F |
| Proline (P, Pro) | T | F | F | T | F |
| Glutamine (Q, Gln) | F | F | F | F | F |
| Arginine (R, Arg) | F | F | F | F | T |
| Serine (S, Ser) | T | F | F | F | F |
| Threonine (T, Thr) | T | F | F | F | F |
| Valine (V, Val) | T | T | F | T | F |
| Tryptophan (W, Trp) | F | F | T | T | F |
| Tyrosine (Y, Tyr) | F | F | T | T | F |
| N-Ethyl β-Alanine (Neb) | T | T | F | T | F |
| Sarcosine (Sar) | T | T | F | T | F |
| α,β-Diaminopropionic Acid | T | T | F | F | T |
| (Adp) | | | | | |
| β-Amino-N-Butyric Acid | T | T | F | T | F |
| (aBut or Bab) | | | | | |
| β-Aminoisobutyric Acid (Bai) | T | T | F | T | F |
| α-Aminoisobutyric Acid | T | T | F | T | F |
| (Aib) | | | | | |
| γ-Aminobutyric Acid (Gab) | T | T | F | T | F |
| alpha-Aminobutyric Acid | T | T | F | T | F |
| (Anb) | | | | | |
| N-Methyl Alanine (Nma) | T | T | F | T | F |
| N-Ethyl Glycine (Neg) | T | T | F | T | F |
| Allothreonine (Alt) | T | F | F | F | F |
| Homoserine (Hse) | T | F | F | F | F |
| 4-Amino-3-Hydroxybutyric | T | F | F | F | F |
| Acid (Hga) | | | | | |
| 2,4-Diaminobutyric Acid | T | F | F | F | T |
| (Dab) | | | | | |
| Hydroxyproline (Hyp) | F | F | F | F | F |
| Isovaline (Iva) | F | T | F | T | F |
| Norvaline (Nor) | F | T | F | T | F |
| L-Cyclopropylglycine (Cpg) | T | T | F | T | F |
| N-Propylglycine (Npg) | F | T | F | T | F |
| Homocysteine (Hcy) | F | F | F | T | F |
| Pipecolic Acid (Pip) | F | T | F | T | F |
| Ornithine (Orn) | F | F | F | T | T |
| Tertleucine (Tle) | F | T | F | T | F |
| Alloisoleucine (Ali) | F | T | F | T | F |
| Norleucine (Nle) | F | T | F | T | F |
| 2-Aminoheptanoic acid (Ahe) | F | T | F | T | F |
| Citrulline (Cit) | F | F | F | F | F |

Amino acid names are provided in the first column with the single letter code and an optional three letter code (if applicable) in parentheses. Amino acid physicochemical properties (“Small”, “Aliphatic”, “Aromatic”, “Hydrophobic”, “Charged”) are indicated as T (“True”) or F (“False”) and are provided for guidance in selecting amino acids for use in heptad repeats described herein. The description “Charge” refers to the state of the amino acid at physiologic pH, pH 7.4. For instance, aspartic acid (D, Asp) is indicated as a small amino acid with charge, e.g., charge at physiologic pH (pH 7.4).

In certain embodiments of heptad repeats of formula (AAₐ-AA_{b}-AA_{c}-AA_{d}-AAₑ-AA_{f}-AA_{g})ₑ, each occurrence of AAₐ and AA_{d} is independently selected from hydrophobic amino acids, more preferably aliphatic amino acids, including but not limited to leucine, isoleucine, norleucine, valine, norvaline, T-leucine, allo-isoleucine, as well as N-alkylated amino acids, such as N-propyl glycine, methionine and alkylated alcohols, such as O-methyl serine. In preferred embodiments of heptad repeats of formula (AAₐ-AA_{b}-AA_{c}-AA_{d}-AAₑ-AA_{f}-AA_{g})ₑ each occurrence of AAₐ and AA_{d} is independently selected from leucine, isoleucine and norleucine.

In certain embodiments of heptad repeats of formula (AAₐ-AA_{b}-AA_{c}-AA_{d}-AAₑ-AA_{f}-AA_{g})ₑ, each occurrence of AA_{b}, AA_{c} and AA_{f} is independently selected from small amino acids including but not limited to alanine, cysteine, aspartic acid, glycine, asparagine, proline, serine, threonine, valine, alpha-amino-butyric acid and norvaline. In preferred embodiments of heptad repeats of formula (AAₐ-AA_{b}-AA_{c}-AA_{d}-AAₑ-AA_{f}-AA_{g})ₑ, each occurrence of AA_{b}, AA_{c} and AA_{f} is independently selected from alanine, proline and serine.

In certain embodiments of heptad repeats of formula (AAₐ-AA_{b}-AA_{c}-AA_{d}-AAₑ-AA_{f}-AA_{g})ₑ, each occurrence of AAₑ and AA_{g} is independently selected from charged amino acids, including but not limited to aspartic acid, glutamic acid, lysine, arginine, ornithine, sulfo-serine and phosphoserine. In preferred embodiments of heptad repeats of formula (AAₐ-AA_{b}-AA_{c}-AA-AAₑ-AA_{f}-AA_{g})ₑ, each occurrence of AAₑ and AA_{g} is independently selected from aspartic acid, glutamic acid, lysine, arginine and ornithine.

Non-limiting examples of heptad repeats of formula (AAₐ-AA_{b}-AA_{c}-AA_{d}-AAₑ-AA_{f}-AA_{g})ₑ, include but are not limited to (I-A-A-L-E-S-K)ₑ(SEQ ID NO:327), (I-A-A-L-K-S-K)ₑ(SEQ ID NO:328), (I-A-A-L-E-S-E)ₑ, (SEQ ID NO:329) (I-A-A-L-K-S-E)ₑ(SEQ ID NO:330), (V-A-A-L-K-A-E)ₑ(SEQ ID NO:331), (I-A-A-L-K-A-E)ₑ(SEQ ID NO:332), (L-A-A-L-K-A-E)ₑ(SEQ ID NO:333), (V-S-A-L-K-A-E)ₑ(SEQ ID NO:334), (I-S-A-L-K-A-E)ₑ, (SEQ ID NO:335) (L-S-A-L-K-A-E)ₑ(SEQ ID NO:336), (V-A-S-L-K-A-E)ₑ(SEQ ID NO:337), (I-A-S-L-K-A-E)ₑ(SEQ ID NO:338), (L-A-S-L-K-A-E)ₑ(SEQ ID NO:339), (V-S-S-L-K-A-E)ₑ(SEQ ID NO:340), (I-S-S-L-K-A-E)ₑ(SEQ ID NO:341), (L-S-S-L-K-A-E)ₑ(SEQ ID NO:342), (V-A-A-L-K-S-E)ₑ(SEQ ID NO:343), (L-A-A-L-K-S-E)ₑ(SEQ ID NO:344), (V-S-A-L-K-S-E)ₑ(SEQ ID NO:345), (I-S-A-L-K-S-E)ₑ(SEQ ID NO:346), (L-S-A-L-K-S-E)ₑ(SEQ ID NO:347), (V-A-S-L-K-S-E)ₑ (SEQ ID NO:348) and (I-A-S-L-K-S-E)ₑ(SEQ ID NO:349).

For clarity, unless otherwise specified, heptad repeats used in extension sequences or linkers described herein can start and end at any position. For example, (AAₐ-AA_{b}-AA_{c}-AA_{d}-AAₑ-AA_{f}-AA_{g})ₑ, may also be written (AA_{c}-AA_{d}-AAₑ-AA_{f}-AA_{g}-AAₐ-AA_{b})ₑ or (AAₑ-AA_{f}-AA_{g}-AAₐ-AA_{b}-AA_{c}-AA_{d})ₑ. In non-limiting examples, the heptad repeat (I-A-A-L-E-S-K)ₑ used in extensions (E1 and/or E2) may be substituted with the heptad repeat (E-S-K-I-A-A-L)ₑ.

Each occurrence of AAₐ, AA_{b}, AA_{c}, AA_{d}, AAₑ, AA_{f} and AA_{g} for a heptad repeat of formula (AAₐ-AA_{b}-AA_{c}-AA_{d}-AAₑ-AA_{f}-AA_{g})ₑ may be the same or different. Non-limiting examples of heptad repeats of formula (AAₐ-AA_{b}-AA_{c}-AA-AAₑ-AA_{f}-AA_{g})ₑ wherein e is 2 and each occurrence of AAₑ AAₑ are the same or different is shown here for clarity: I-A-A-L-K-S-K-I-A-A-K-E-S-E (SEQ ID NO:373), I-A-A-L-K-S-K-I-A-A-K-E-S-K (SEQ ID NO:374) and I-A-A-L-E-S-E-I-A-A-K-E-S-K (SEQ ID NO:375).

An unexpected finding disclosed herein is that vaccines comprising certain heptad repeats of formula (AAₐ-AA_{b}-AA_{c}-AA-AAₑ-AA_{f}-AA_{g})ₑ comprising L-amino acids induced antibodies that bound to certain peptide sequences derived from endogenous proteins, including myosin proteins, whereas vaccines comprising certain heptad repeats of formula (AAₐ-AA_{b}-AA_{c}-AA_{d}-AAₑ-AA_{f}-AA_{g})ₑ comprising one or more D-amino acids did not induce antibodies against peptide sequences derived from myosin proteins. Based on these unexpected findings, in certain preferred embodiments the heptad repeat of formula (AA_{H}-AA_{P}-AA_{P}-AA_{H}-AA_{P}-AA_{P}-AA_{P})ₑ, which may be written (AAₐ-AA_{b}-AA_{c}-AA_{d}-AAₑ-AA_{f}-AA_{g})ₑ, comprises amino acids selected from one or more D amino acids. Non-limiting examples of heptad repeats of formula (AAₐ-AA_{b}-AA_{c}-AA_{d}-AAₑ-AA_{f}-AA_{g})ₑ comprising D-amino acids, include but are not limited to (I-A-A-L-E-S-K)ₑ (SEQ ID NO:379), (I-A-A-L-K-S-K)ₑ (SEQ ID NO: 380), (I-A-A-L-E-S-E)ₑ(SEQ ID NO: 381), (I-A-A-L-K-S-E)ₑ (SEQ ID NO:382), (V-A-A-L-K-A-E)ₑ (SEQ ID NO:383), (I-A-A-L-K-A-E)ₑ (SEQ ID NO:384), (L-A-A-L-K-A-E)ₑ (SEQ ID NO:385), (V-S-A-L-K-A-E)ₑ (SEQ ID NO:386), (I-S-A-L-K-A-E)ₑ (SEQ ID NO:387), (L-S-A-L-K-A-E)ₑ (SEQ ID NO:388), (V-A-S-L-K-A-E)ₑ (SEQ ID NO:389), (I-A-S-L-K-A-E)ₑ (SEQ ID NO:390), (L-A-S-L-K-A-E)ₑ (SEQ ID NO:391), (V-S-S-L-K-A-E)ₑ (SEQ ID NO:392), (I-S-S-L-K-A-E)ₑ (SEQ ID NO:393), (L-S-S-L-K-A-E)ₑ (SEQ ID NO:394), (V-A-A-L-K-S-E)ₑ (SEQ ID NO:395), (L-A-A-L-K-S-E)ₑ (SEQ ID NO:396), (V-S-A-L-K-S-E)ₑ (SEQ ID NO:397), (I-S-A-L-K-S-E)ₑ (SEQ ID NO:398), (L-S-A-L-K-S-E)ₑ (SEQ ID NO:399), (V-A-S-L-K-S-E)ₑ (SEQ ID NO:400), (I-A-S-L-K-S-E)ₑ (SEQ ID NO:401), wherein, italicized letters here indicate that the amino acids are D-amino acids, optionally wherein up to 6 out of 7 of the amino acids of the heptad repeat may be L-amino acids, e.g., 1 of the 7 amino acids of a heptad repeat are D-amino acids.

An additional notable finding was that for certain heptad repeats of formula (AA_{H}-AA_{P}-AA_{P}-AA_{H}-AA_{P}-AA_{P}-AA_{P})ₑ, e.g., (AAₐ-AA_{b}-AA_{c}-AA_{d}-AAₑ-AA_{f}-AA_{g})ₑ comprising L-amino acids that induced antibodies that bound to certain peptide sequences derived from myosin proteins, changing the directionality of the of the heptad repeat, e.g., from (AAₐ-AA_{b}-AA_{c}-AA_{d}-AAₑ-AA_{f}-AA_{g})ₑ to (AA_{g} AA_{f}-AAₑ-AA_{d}-AA_{c}-AA_{b}-AAₐ)ₑ, led to heptad repeats that did not induce antibodies against peptide sequences derived from myosin proteins. Non-limiting examples of heptad repeats that have alternative directionality to heptad repeats found in myosin include but are not limited to (K-S-E-L-A-A-1)ₑ (SEQ ID NO:350), (K-S-K-L-A-A-I)ₑ (SEQ ID NO:351), (E-S-S-L-A-A-I)ₑ (SEQ ID NO:352), (E-S-K-L-A-A-1)ₑ (SEQ ID NO:353), (E-A-K-L-A-A-V)ₑ (SEQ ID NO:354), (E-A-K-L-A-A-I)ₑ (SEQ ID NO:355), (E-A-K-L-A-A-L)ₑ (SEQ ID NO:356), (E-A-K-L-A-S-V)ₑ (SEQ ID NO:357), (E-A-K-L-A-S-1)ₑ (SEQ ID NO:358), (E-A-K-L-A-S-L)ₑ (SEQ ID NO:359), (E-A-K-L-S-A-V)ₑ (SEQ ID NO:360), (E-A-K-L-S-A-I)ₑ (SEQ ID NO:361), (E-A-K-L-S-A-L)ₑ (SEQ ID NO:362), (E-A-K-L-S-S-V)ₑ (SEQ ID NO:363), (E-A-K-L-S-S-I)ₑ (SEQ ID NO:364), (E-A-K-L-S-S-L)ₑ (SEQ ID NO:365), (E-S-K-L-A-A-V)ₑ (SEQ ID NO:366), (E-S-K-L-A-A-I)ₑ (SEQ ID NO:367), (E-S-K-L-A-A-L)ₑ (SEQ ID NO:368), (E-S-K-L-A-S-V)ₑ (SEQ ID NO:369), (E-S-K-L-A-S-I)ₑ (SEQ ID NO:370), (E-S-K-L-A-S-L)ₑ (SEQ ID NO:371), (E-S-K-L-S-A-V)ₑ (SEQ ID NO:372), (E-S-K-L-S-A-I)ₑ (SEQ ID NO:373).

In some embodiments of vaccines, the one or more peptide antigen conjugates may each comprise an extension further comprising a heptad repeat of formula (AA_{H}-AA_{P}-AA_{P}-AA_{H}-AA_{P}-AA_{P}-AA_{P})ₑ, e.g., (AAₐ-AA_{b}-AA_{c}-AA_{d}-AAₑ-AA_{f}-AA_{g})ₑ, that may be the same or different. In non-limiting examples, a first peptide antigen conjugate may comprise an extension (I-A-A-L-E-S-K)ₑ (SEQ ID NO:327) and a second peptide antigen conjugate may comprise an extension (I-A-A-L-E-S-K)ₑ (SEQ ID NO:327) or (I-A-A-L-K-S-E)ₑ (SEQ ID NO:330), or a first peptide antigen conjugate may comprise an extension (I-A-A-L-E-S-E)ₑ (SEQ ID NO:329) and a second peptide antigen conjugate may comprise an extension (I-A-A-L-K-S-K)ₑ (SEQ ID NO: 377) or (I-A-A-L-R-S-R)ₑ (SEQ ID NO: 378) 327). In general, for vaccines comprising one or more peptide antigen conjugates with extensions comprising heptad repeats of formula (AAₐ-AA_{b}-AA_{c}-AAₐ-AAₑ-AA_{f}-AA_{g})ₑ, the amino acids at the positions AAₑ and AA_{g}, are selected such that the net charge is zero. For example, for a vaccine comprising a first conjugate and a second conjugate, wherein the first conjugate comprises an extension with the sequence (AAₐ-AA_{b}-AA_{c}-AAₐ-E-AA_{f}-E)ₑ, e.g., (I-A-A-L-E-S-E) (SEQ ID NO:329), the second conjugate is chosen such that the net charge is zero and would be typically selected from, (AAₐ-AA_{b}-AA_{c}-AAₐ-K-AA_{f}-K)ₑ, e.g., (I-A-A-L-K-S-K) (SEQ ID NO: 328), where K (lysine) may be optionally substituted with R (arginine) or ornithine.

For vaccines used for inducing antibody responses, the linker molecule joining extensions comprising heptad repeats to the peptide antigen was found to impact the magnitude and quality of antibody responses. Accordingly, vaccines comprising short and/or rigid amino acid linkers joining the heptad repeat based extensions (E1 or E2) to the peptide antigen (A) resulted in higher titers of functional antibodies than vaccines comprising longer and/or flexible linkers joining the heptad repeat to the peptide antigen (A). A non-limiting explanation is that the rigid linkers constrain the antigen and enable enhanced B-cell receptor clustering as compared with longer and/or flexible linkers. Therefore, in certain preferred embodiments of vaccines comprising extensions (E1 or E2) selected from heptad repeats, the heptad repeat is linked to other components of the vaccine either through no linker (i.e., the linker is absent) or the linker is selected from short and/or rigid linkers including but not limited to single amino acids selected from, but not limited to alanine, proline, β-alanine, N-ethyl-β-alanine, hydroxyproline, pipecolic Acid and stachydrine.

### Spacer (B)

The spacer (B) is an optional component of amphiphiles that links the solubilizing block (S) to the hydrophobic block (H) either directly or via a Linker (U), e.g., wherein the amphiphile has the structure S-B-H or S-B-U-H. The spacer (B) may comprise any one or more of the following: amino acids, including non-natural amino acids; hydrophilic polymers, e.g., polymers based on ethylene oxide (PEG), acrylate, methacrylate, acrylamide or methacrylamide based monomers; alkane chains; or the like; or combinations thereof. The spacer (B) may be linked to the solubilizing block (S) and hydrophobic block (H) through any suitable means, e.g., directly or indirectly via linkers, though the linkages typically comprise covalent bonds, e.g., amide bonds.

In some embodiments, the spacer (B) functions to provide distance, i.e., space, between the heterologous molecules, S and H. In other embodiments, the spacer (B) functions to impart hydrophobic or hydrophilic properties. In still other embodiments, the composition of the spacer may be selected to impart rigidity or flexibility. In other embodiments, the composition of the spacer may be selected for recognition by enzymes and promote degradation.

In some embodiments, the spacer (B) is a hydrophilic polymer, with monomer units selected from acrylates, (meth)acrylates, acrylamides, (meth)acrylamides, allyl ethers, vinyl acetates, vinyl amides, substituted styrenes, amino acids, acrylonitrile, heterocyclic monomers (e.g., ethylene oxide), saccharides, phosphoesters, phosphonamides, sulfonate esters, sulfonamides, or combinations thereof.

In some embodiments, the spacer (B) is a peptide sequence between about 1 to 45 amino acids in length, such as 1, 2, 3, 4, 5, 6, 7, 8, 9, 10, 11, 12, 13, 14, 15, 16, 17, 18, 19, 29, 21, 22, 23, 24, 25, 26, 27, 28, 29, 30, 31, 32, 33, 34, 35, 36, 37, 38, 39, 40, 41, 42, 43, 44 or 45 amino acids, typically no more than 45 amino acids in length, that is linked to the hydrophobic block (H) and solubilizing block (S) through, e.g., an amide bond formed between the N- and C-terminal carboxyl group of the spacer (B), respectively. The amide bond between the spacer (B) and the solubilizing block (S) and/or hydrophobic block (H) may be recognized by enzymes or may be selected for resistance to enzyme-mediated hydrolysis.

In other embodiments, the spacer (B) is a hydrophilic polymer comprising monomer units selected from non-natural, hydrophilic monomers, e.g., ethylene oxide (PEG), HPMA, or HEMA, that is about 1 to 48 monomers in length (i.e. degree of polymerization), such as 1, 2, 3, 4, 5, 6, 7, 8, 9, 10, 11, 12, 13, 14, 15, 16, 17, 18, 19, 29, 21, 22, 23, 24, 25, 26, 27, 28, 29, 30, 31, 32, 33, 34, 35, 36, 37, 38, 39, 40, 41, 42, 43, 44, 45, 46, 47 or 48 monomers, typically no more than 48 monomers in length, that is linked to the hydrophobic block (H) and solubilizing block (S) either directly or through linkers.

Specific compositions of spacers that lead to unexpected improvements in biological activity are described throughout the specification. Note: spacer groups (B) and solubilizing blocks (S) may both comprise hydrophilic polymers (e.g., hydrophilic poly(amino acids); hydrophilic methacrylate-based polymers, such as HEMA; hydrophilic methacrylamide-based polymers, such as HPMA, PEG, etc.); however, the distinction between S and B is based in part on function and called attention to in specific examples of amphiphiles.

### Linker (U)

A linker (U) optionally joins solubilizing block (S) fragments (S-[B]-U1) to hydrophobic block (H) fragments (U2-H) through the reaction of U1 with U2 to form amphiphiles (S-[B]-U-H).

A linker (U) also, independently of the amphiphile linker U, joins peptide antigen conjugate fragments ([S]-[E1]-A-[E2]-U1 or U1-[E1]-A-[E2]-[S]) to hydrophobic block (H) fragments (U2-H) through the reaction of U1 with U2 to form peptide antigen conjugates ([S]-[E1]-A-[E2]-U-H or H-U-[E1]-A-[E2]-[S]).

While peptide antigens (A) may be joined directly to hydrophobic blocks (H), i.e., A-H, or via an extension, i.e., A-E2-H (or H-E1-A), entirely on-resin by solid-phase peptide synthesis, it may be beneficial under certain circumstances to produce the antigen (A) and hydrophobic block (H) as separate fragments comprising Linker Precursor U1 ([S]-[E1]-A-[E2]-U1 or U1-[E1]-A-[E2]-[S]) and Linker Precursor U2 (U2-H), which may be joined on-resin or in solution to yield [S]-[E1]-A-[E2]-U-H (or H-U-[E1]-A-[E2]-[S]).

Similarly, while solubilizing blocks (S) on the amphiphile may be joined directly to hydrophobic blocks (H), i.e., S-H, or via a spacer, i.e., S-B-H, entirely on-resin by solid-phase peptide synthesis, it may be beneficial under certain circumstances to produce the solubilizing block (S) and hydrophobic block (H) as separate fragments comprising Linker Precursor U1 (S-[B]-U1) and Linker Precursor U2 (U2-H), which may be joined on-resin or in solution to yield S-[B]-U-H.

In preferred embodiments, the Linker Precursors used to form Linker U are selected for site-selectivity, i.e., a reaction only takes place between U1 and U2 and between no other groups. In some embodiments, Linker Precursor U1 comprises an activated carboxylic acid and is reacted with a Linker Precursor U2 that comprises an amine to form Linker U comprising an amide; or, U1 comprises an amine and is reacted with U2 that comprises an activated carboxylic acid to form Linker U comprising an amide. In some embodiments, Linker Precursor U1 comprises a maleimide and is reacted with Linker Precursor U2 that comprises a thiol to form a Linker U comprising a thioether bond; or, U1 comprises a thiol and is reacted with U2 that comprises a maleimide to form a Linker U comprising a thioether bond. In some embodiments, Linker Precursor U1 comprises an azide and is reacted with Linker Precursor U2 that comprises an alkyne to form a Linker U that comprises a triazole; or, U1 comprises an alkyne and is reacted with a U2 that comprises an azide to form a Linker US comprising a triazole.

In preferred embodiments, the amphiphile of formula S-[B]-U-H is joined together by linking a solubilizing block fragment (S-[B]-U1) to a hydrophobic block fragment (U2-H), wherein the Linker Precursor U1 comprises a strained alkyne (e.g., dibenzocyclooctyne (DBCO), bicyclononyne (BCN) or the like) that is reacted with Linker Precursor U2 which comprises an azide to form the Linker U that comprises a triazole.

In preferred embodiments, the peptide antigen conjugates of formulas [S]-[E1]-A-[E2]-U-H or H-U-[E1]-A-[E2]-[S] are joined together by linking a peptide antigen fragment [S]-[E1]-A-[E2]-U1 or U1-[E1]-A-[E2]-[S] to a hydrophobic block fragment (U2-H), wherein the Linker Precursor U1 comprises a strained alkyne (e.g., dibenzocyclooctyne (DBCO), bicyclononyne (BCN) or the like) that is reacted with Linker Precursor U2 which comprises an azide to form the Linker U which comprises a triazole.

In other preferred embodiments, Linker Precursor U1 comprises an azide that is reacted with the Linker Precursor U2 that comprises a strained alkyne (e.g., dibenzocyclooctyne (DBCO), bicyclononyne (BCN) or the like) to form the Linker U which comprises a triazole. In non-limiting examples, the Linker Precursor U2 comprising DBCO is linked to the hydrophobic block (H) via a suitable linker X (e.g., DBCO-NHS, CAS number 1353016-71-3) and the Linker Precursor U1 (e.g. azido acid, such as azidopentanoic acid; azido amino acid, such as azido-lysine (abbreviated Lys(N3), CAS number 159610-92-1; or, azido amine, such as azido-butylamine) is linked to the solubilizing block fragment (S-[B]-U1) or peptide antigen fragment ([S]-[E1]-A-[E2]-U1 or U1-[E1]-A-[E2]-[S]) via a suitable linker X.

In preferred embodiments, the Linker U preferably comprises an amide, thioether or triazole.

### Dendron Amplifier

Dendron amplifiers are a specific type of linker moiety that functions to increase the valency (i.e., the number) of groups present on any components of amphiphiles, peptide antigen conjugates or drug molecule conjugates described herein. For instance, in preferred embodiments of solubilizing blocks (S), dendron amplifiers are used to increase the valency of solubilizing groups (referred to as “SG” in formulae) that are present on the surface of the solubilizing block (S). In other embodiments, dendron amplifiers are used to increase the valency of solubilizing blocks (S) and spacers (B) linked to a hydrophobic block (H).

Dendron amplifiers (also referred to as “dendrons”) are regularly branched molecules that are often symmetric and typically comprise repeating units of monomers that comprise three or more functional groups (FG) and a branch point. Dendron amplifiers may be expressed by the formula, (FG′)-T-(FGt)d, wherein FG′ and FGt are the focal point and terminal functional groups, respectively, which are selected from any suitable functional group; T is any suitable linker and “d” is any integer greater than 1, typically between 2 to 32, though, more preferably between 2 and 8, such as 2, 3, 4, 5, 6, 7, and 8. The multiple by which dendron amplifiers increase the terminal functional group (FGt) can be expressed as FGt=β^{γ}, wherein β is the number of branches that occur for each generation of the dendron and the symbol γ is the number of generations, wherein the number of branches is any integer, though, typically between 2 to 6, and the number of generations is any integer, though, typically between 1 to 10. Terminal functional groups present on solubilizing blocks that are free (i.e., unreacted), may also be referred to as solubilizing groups (SG).

Dendron amplifiers may comprise repeats of a monomer comprising a first functional group (FG1) and a second functional group (FG2), wherein the first functional group is reactive towards the second functional group. For instance, a non-limiting example of a 2^{nd} generation dendron amplifier with β=2 comprising repeats of a monomer comprising a first functional group (FG1) and a second functional group (FG2), wherein the first functional group is reactive towards the second functional group, is shown here for clarity:

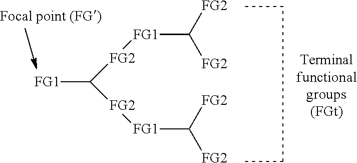

Wherein, the first functional group at the starting point is also referred to as the focal point functional group (FG′) and the terminal FG2 are referred to as the terminal functional groups or FGt.

A non-limiting example of a 3^{rd} generation dendron formed from monomers comprising a first and second functional group wherein β=2 is shown here for clarity:

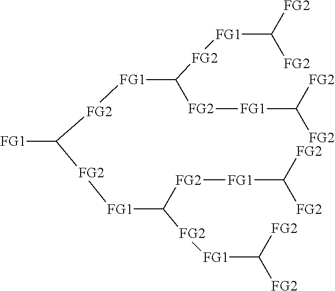

A non-limiting example of a 2^{nd} generation dendron amplifier with β=3 comprising repeats of a first monomer comprising a first functional group (FG1) and a second functional group (FG2), wherein the first functional group is reactive towards the second functional group, is shown here for clarity:

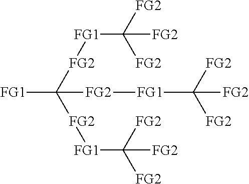

Monomers comprising a first functional group and a second functional group, wherein the first functional group is reactive towards the second functional group, and the monomer comprises at least one first functional group and two or more second functional groups may be selected from any suitable monomer. Non-limiting examples include FG1-(CH₂)_{y2}CH(R¹)₂, FG1-(CH₂)_{y2}C(R¹)₃, FG1-(CH₂CH₂O)_{y2}CH(R¹)₂, FG1-(CH₂CH₂O)_{y2}C(R¹)₃, FG1-CH(R¹)₂, FG1-C(R)₃, wherein R¹ is independently selected from (CH₂)_{y3}-FG2, (OCH₂CH₂)_{y3}-FG2 or CH₂(OCH₂CH₂)_{y3}-FG2) and y2 and y3 are each an integer number of repeating units selected from between 1 to 6.

A non-limiting example of FG1-CH(R¹)₂, wherein FG1 is NH₂, R¹ is CH₂(OCH₂CH₂)_{y3}-FG2, y3 is 1 and FG2 is COOH is shown here for clarity:

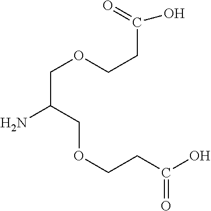 [SMILES: NC(COCCC(=O)O)COCCC(=O)O]

Wherein the above monomer is used to produce a 2^{nd} generation amplifying linker, the structure is:

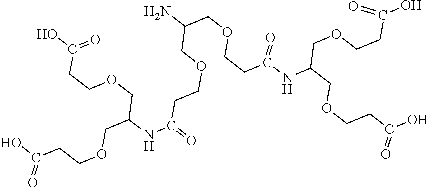 [SMILES: NC(COCCC(=O)NC(COCCC(=O)O)COCCC(=O)O)COCCC(=O)NC(COCCC(=O)O)COCCC(=O)O]

Additional non-limiting examples of monomers comprising a first functional group and a second functional group, wherein the first functional group is reactive towards the second functional group, and the monomer comprises at least one first functional group and two or more second functional groups include FG1-(CH₂)_{y2}N(R²)₂, FG1-(CH₂CH₂O)_{y2}CH₂CH₂N(R²)₂, wherein R² is independently selected from (CH₂)_{y3}-FG2, (CH₂CH₂O)_{y3}(CH₂)_{y4}-FG2, (CH₂OCH₂CH₂)_{y3}-FG2) and y2, y3 and y4 are each an integer of repeating units selected from between 1 to 6. Note: in the above example, FG′ is an amine and the 4 FGt are carboxylic acids.

A non-limiting example of FG1-(CH₂CH₂O)_{y1}CH₂CH₂N(R²)₂, wherein FG1 is NH₂, R² is (CH₂CH₂O)_{y3}(CH₂)_{y4}-FG2, y2 is 2, y3 is 1, y4 is 2 and FG2 is COOH is shown here for clarity:

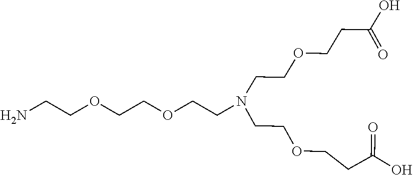 [SMILES: NCCOCCOCCN(CCOCCC(=O)O)CCOCCC(=O)O]

In still additional non-limiting examples of monomers comprising a first functional group and a second functional group, wherein the first functional group is reactive towards the second functional group, and the monomer comprises at least one first functional group and two or more second functional groups include certain amino acids, such as glutamic acid, aspartic acid, lysine or ornithine. A non-limiting example of a 3^{rd} generation lysine dendron is shown here for clarity:

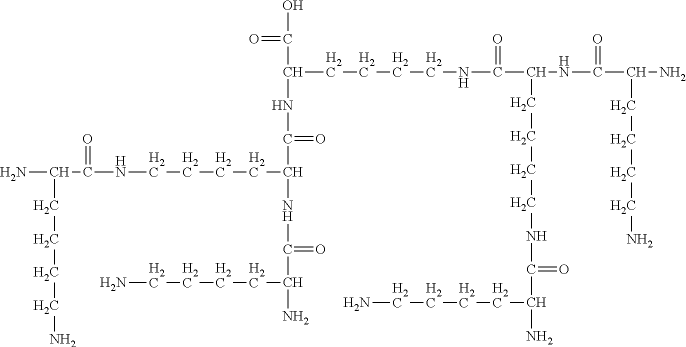 [SMILES: NCCCCC(N)C(=O)NCCCCC(NC(=O)C(N)CCCCN)C(=O)NCCCCC(NC(=O)C(CCCCNC(=O)C(N)CCCCN)NC(=O)C(N)CCCCN)C(=O)O]

Dendron amplifiers may comprise repeats of two monomers, wherein a first monomer comprises three or more first functional groups (FG1) and the second monomer comprises two or more second functional groups (FG2), wherein the first functional group is reactive towards the second functional group. For instance, a non-limiting example of a 2^{nd} generation dendron amplifier with β=2 comprising repeats of a first and second monomer, wherein the first monomer comprises three first functional groups (FG1) and the second monomer comprises two second functional groups (FG2), wherein the first functional group is reactive towards the second functional group, is shown here for clarity:

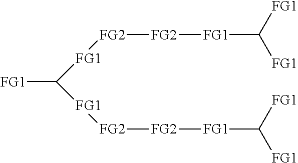

A non-limiting example of a 1^{st} generation dendron amplifier with β=2 comprising repeats of a first and second monomer, wherein the first monomer comprises three first functional groups (FG1) and the second monomer comprises three second functional groups (FG2), wherein the first functional group is reactive towards the second functional group, is shown here for clarity:

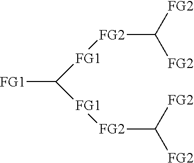

Dendron amplifiers may be use to join together any three or more components of amphiphiles, peptide antigen conjugates and drug molecule conjugates. The focal point functional group (FG′) and the terminal functional groups (FGt) may be further functionalized, i.e., reacted to fit a particular purpose.

In preferred embodiments of amphiphiles of formula S-[B]-[U]-H, the solubilizing block (S) comprises a dendron amplifier wherein the focal point is linked to the hydrophobic block (H) either directly or indirectly via a spacer (B) and/or Linker U and the terminal functional groups (FGt) either are unlinked and serve as the solubilizing groups or are linked to a solubilizing group (SG). Solubilizing groups (SG) are any molecules that are hydrophilic and/or charged; preferred solubilizing groups (SG) are described throughout the specification.

In some embodiments of amphiphiles of formula S-[B]-[U]-H-D, peptide antigen conjugates of formula [S]-[E1]-A-[E2]-[U]-H-D or H-D-[U]-[E1]-A-[E2]-[S]) and drug molecule conjugates of formula H-D, the hydrophobic block (H) comprises a dendron amplifier wherein the focal point is linked to either (i) a solubilizing block (S) either directly or indirectly via a spacer (B) and/or Linker U, (ii) an antigen (A) either directly or indirectly via an extension (E1 or E2) and/or Linker U; or (iii) a drug molecule either directly or via a Linker X1.

In some embodiments, the hydrophobic block (H) comprises a dendron amplifier and the terminal functional groups (FGt) are linked to hydrophobic drug molecules. In such embodiments, the focal point is linked to either (i) a solubilizing block (S) either directly or indirectly via a spacer (B) and/or Linker U, (ii) an antigen (A) either directly or indirectly via an extension (E1 or E2) and/or Linker U; or (iii) is unreacted or capped with a terminal group, such as an acetyl group. Capped or capping refers to the modification of a functional group, such as FGt, to make it less reactive and/or have neutral charge at pH 7.4. For example, an amine may be capped with an activated carboxylic acid (e.g., acetyl chloride) to result in a relatively less reactive amide; or, e.g., a strained alkyne may be capped with an alkyl-azide to result in a relatively less reactive triazole.

### Hydrophobic Block (H)

The hydrophobic block (sometimes designated “H” in formulae) is a molecule with substantially limited water solubility, or is amphiphilic in properties, and capable of assembling into supramolecular structures, e.g., micellar, nano- or micro-particles in aqueous solutions. In certain embodiments, the hydrophobic block (H) is insoluble, or forms micelles, in aqueous solutions at concentrations of about 1.0 mg/mL or less, e.g., about 0.1 mg/mL or about 0.01 mg/mL. In some embodiments, the hydrophobic block is soluble in aqueous solutions at certain concentrations, temperatures and/or pH ranges but becomes insoluble in response to a change in concentration, temperature and/or pH. For instance, in some embodiments, the hydrophobic block is a hydrophobic polymer that is temperature-responsive, i.e., the hydrophobic polymer is soluble in aqueous solutions at temperatures below a transition temperature (Tₜᵣ) but becomes insoluble at temperatures above the transition temperature. Preferred hydrophobic blocks (H) are molecules that have a solubility of at least less than about 1.0 mg/mL, such as less than about 0.1 mg/mL or less than about 0.01 mg/mL, at or near physiologic pH (˜pH 7.4), between about pH 6.5 to pH 8.5 or between about pH 6.0 and pH 9.0, and at or near physiologic temperature (˜37° C.) and physiologic salt concentrations (˜10 g/L) and salt composition.

The hydrophobic block (H) may be chosen from any molecule comprising higher alkanes, cyclic aromatics, fatty acids, compounds deriving from terpenes/isoprenes, or polymers or oligomers that have limited water solubility and/or amphiphilic characteristics.

Exemplary higher alkanes include but are not limited to octane, nonane, decane, undecane, dodecane, tridecane, tetradecane, pentadecane, hexadecane, heptadecane and octadecane. Exemplary cyclic aromatics include but are not limited to phenyl. Exemplary saturated and unsaturated fatty acids include but are not limited to myristic acid, palmitic acid, stearic acid or oleic acid. In some embodiments, the hydrophobic block (H) is a fatty acid, for example myristic acid. In other embodiments, the hydrophobic block (H) comprises a diacyl lipid, such as 1,2-dioleoyl-sn-glycero-3-phosphoethanolamine or 1,2-distearoyl-sn-glycero-3-phosphoethanolamine or a lipopeptide, e.g., Pam2Cys. In some embodiments, the fatty acid or lipid based hydrophobic block (H) may further comprise a PEG. Exemplary compounds deriving from terpenes/isoprene include sterol derivatives, such as cholesterol, and squalene. In some embodiments, the hydrophobic block (H) comprises cholesterol. In some embodiments, the hydrophobic block (H) comprises a saponin, e.g., QS-21.

In some embodiments the hydrophobic block (H) is a linear, branched or brush polymer (or oligomer). The hydrophobic block (H) can be a homopolymer or copolymer. The hydrophobic block (H) can comprise one or many different types of monomer units. The hydrophobic block (H) can be a statistical copolymer or alternating copolymer. The hydrophobic block (H) can be a block copolymer, such as the A-B type, or the polymer can comprise a grafted copolymer, whereby two or more polymers are linked through polymer analogous reaction.

The hydrophobic block (H) may comprise polymers comprising naturally occurring and/or non-natural monomers and combinations thereof.

In some embodiments, the hydrophobic block (H) is selected from natural biopolymers. Natural biopolymers may include peptides (sometimes referred to as poly(amino acids)) which comprise hydrophobic amino acids. Non-limiting examples of hydrophobic amino acids include leucine, isoleucine, norleucine, valine, tryptophan, phenylamine, tyrosine and methionine, as well as hydrophilic amino acids that have been modified, such as by acetylation or benzoylation to have hydrophobic characteristics. Natural biopolymers that are water soluble in their native form may be used but must be modified chemically to make such natural biopolymers water insoluble and suitable for use as hydrophobic block (H). For example, biopolymers which comprise of hydrophilic amino acids, such as glutamic acid or lysine residues may be modified at the gamma carboxyl or epsilon amine groups, respectively, for the attachment of a hydrophobic molecule, such as a hydrophobic drug molecule, to increase the hydrophobicity of the resulting modified biopolymer. Similarly, biopolymers can be selected from hydrophilic polysaccharides, which may include but are not limited to glycogen, cellulose, dextran, alginate and chitosan, but such polysaccharides should be modified chemically, for example via acetylation or benzoylation of hydrophilic functional groups to render the resulting modified polysaccharide water insoluble. In still further embodiments the hydrophobic block comprises monomers selected from lactic acid and/or glycolic acid.

Monomers comprising the hydrophobic block (H) can be selected from acrylates, (meth)acrylates, acrylamides, (meth)acrylamides, allyl ethers, vinyl acetates, vinyl amides, substituted styrenes, amino acids, acrylonitrile, heterocyclic monomers (e.g., ethylene oxide), saccharides, phosphoesters, phosphonamides, sulfonate esters, sulfonamides, or combinations thereof. Specific examples of (meth)acrylates and (meth)acrylamides include benzyl methacrylamide (BnMAM) and benzyl methacrylate (BnMA), respectively.

Certain monomers described herein as hydrophobic monomers may be water soluble under certain conditions but are hydrophobic and water insoluble at certain conditions in aqueous solutions. Non-limiting examples include temperature-responsive monomers, such as N-isopropylmethacrylamide (NIPMAM); a homopolymer comprising entirely of NIPMAM may be water soluble at room temperature but may become insoluble and form particles at elevated temperatures. Such distinctions are made to facilitate description of certain embodiments. In some embodiments, the hydrophobic block comprises a majority of monomer units selected from hydrophobic monomers that are temperature-responsive (sometimes referred to as “temperature-responsive monomers”), such as NIPAM, NIPMAM, N,N′-diethylacrylamide (DEAAM), N-(L)-(1-hydroxymethyl)propyl methacrylamide (HMPMAM), N,N′-dimethylaminoethylmethacrylate (DMEMA), N-(N-ethylcarbamido)propylmethacrylamide, N-vinylisobutyramide (PNVIBA), N-vinyl-n-butyramide (PNVBA), N-acryloyl-N-propylpiperazine (PNANPP), N-vinylcaprolactam (PVCa), DEGMA, TEGMA, or poly(amino acids) or γ-(2-methoxyethoxy)esteryl-L-glutamate. In still other embodiments, the hydrophobic block (H) may comprise monomers of ethylene oxide, propylene oxide or combinations thereof.

Hydrophobic blocks (H) comprising a polymer typically comprise hydrophobic monomers and one or more other types of monomers, such as reactive monomers optionally linked to a drug molecule, spacer monomers and/or charged monomers. In some embodiments of hydrophobic blocks (H) comprising a polymer (or oligomer), a majority of monomer units are selected from hydrophobic monomers. In other embodiments of hydrophobic blocks (H) comprising a polymer (or oligomer), a majority of monomer units are selected from reactive monomers linked to hydrophobic drug molecules. In still other embodiments of hydrophobic blocks (H) comprising a polymer (or oligomer), the polymer comprises hydrophobic monomers and reactive monomers linked to hydrophobic drug molecules. In still further embodiments of hydrophobic blocks (H) comprising a polymer (or oligomer), the polymer comprises hydrophobic monomers and charged monomers and optionally reactive monomers linked to hydrophobic drug molecules.

In preferred embodiments, the hydrophobic block (H) comprises a polymer (or oligomer) that comprises hydrophobic monomers that further comprise aryl groups. In certain embodiments, the hydrophobic block (H) comprises heteroaryl groups. In still other embodiments, the aryl or heteroaryl groups of the hydrophobic block (H) comprise an amino substituent. The present inventors found that hydrophobic blocks (H) comprising aminoaryl or aminoheteroaryl groups lead to improved manufacturability and solubility in water-miscible solvents. The present inventors also found that amphiphiles with hydrophobic blocks (H) comprising aromatic amines lead to formation of stable particles with low CMC.

In preferred embodiments, the hydrophobic block (H) comprises monomers that comprise aryl or heteroaryl groups. Exemplary aryl groups (sometimes referred to as “aromatics” or “aromatic rings”) include but are not limited to phenyl, naphthyl, and quinolinyl. Non-limiting examples include:

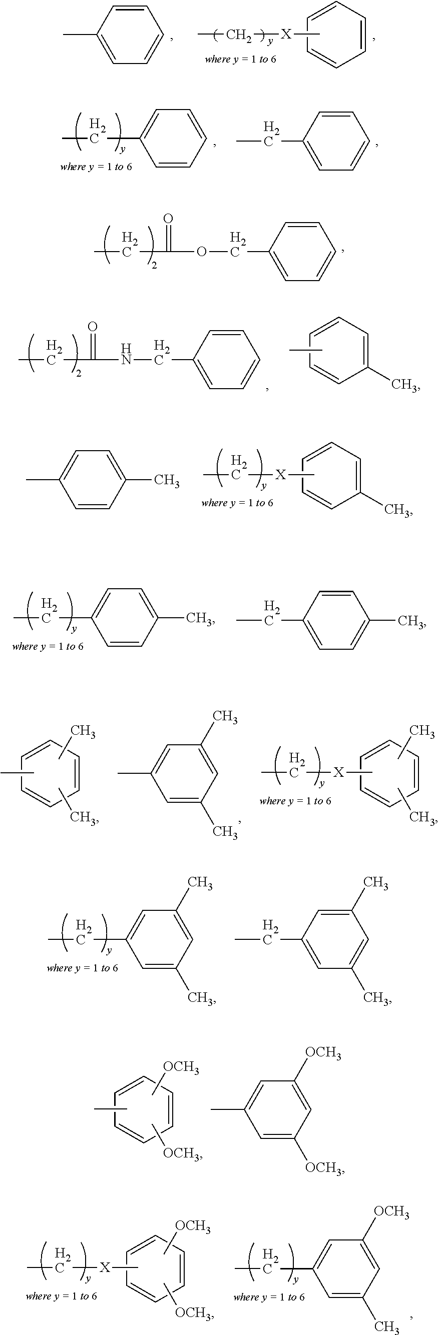

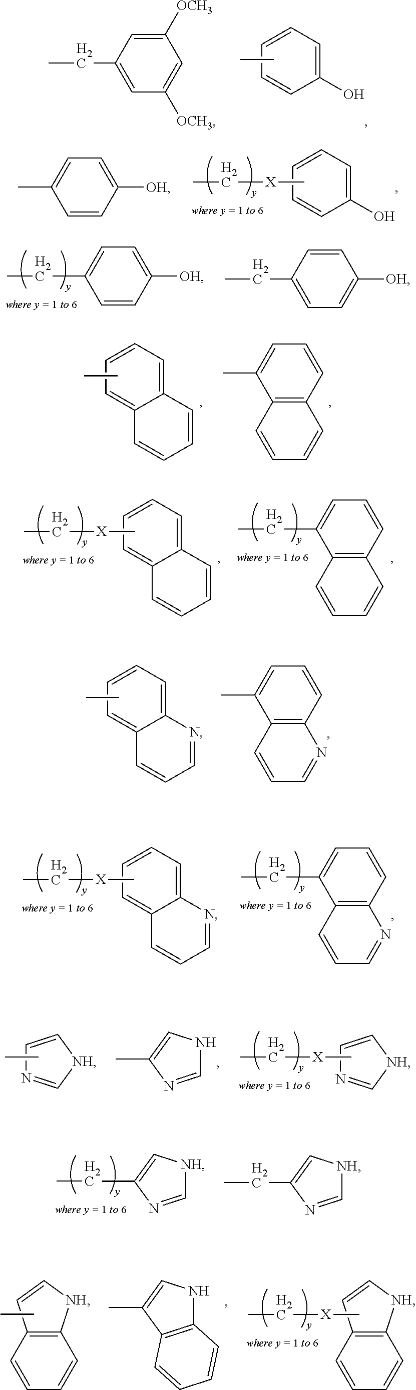

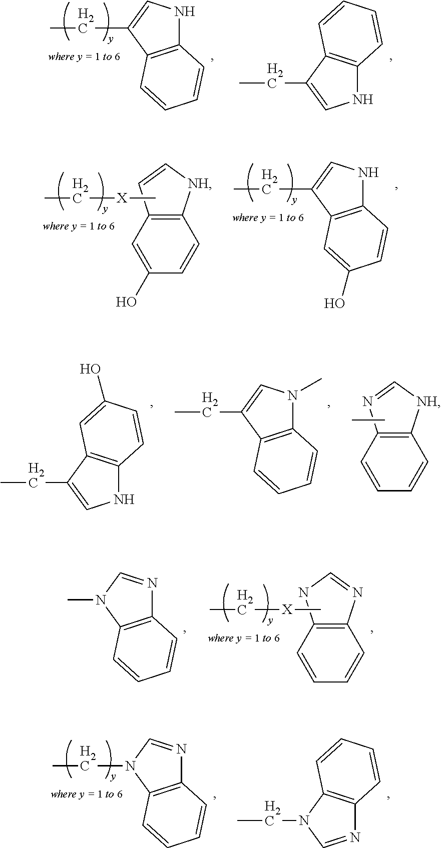

wherein X is any suitable linker molecule and y is an integer value, typically between 1 and 6.

In preferred embodiments, aryl or heteroaryl groups include but are not limited to

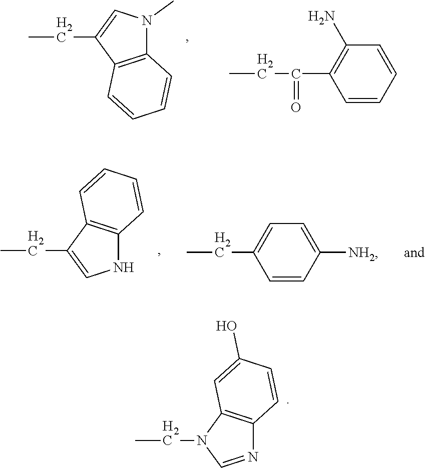

Furthermore, in the aforementioned aryl or heteroaryl groups one or more hydrogen atoms may be substituted for one or more fluorine atoms. In certain embodiments, the hydrophobic block comprises fluorinated aliphatic, aryl or heteroaryl groups, wherein one or more hydrogen atoms of the aforementioned groups comprising the hydrophobic monomer may be substituted for one or more fluorine atoms. The following non-limiting examples of fluorinated aryl groups may be present in hydrophobic monomers:

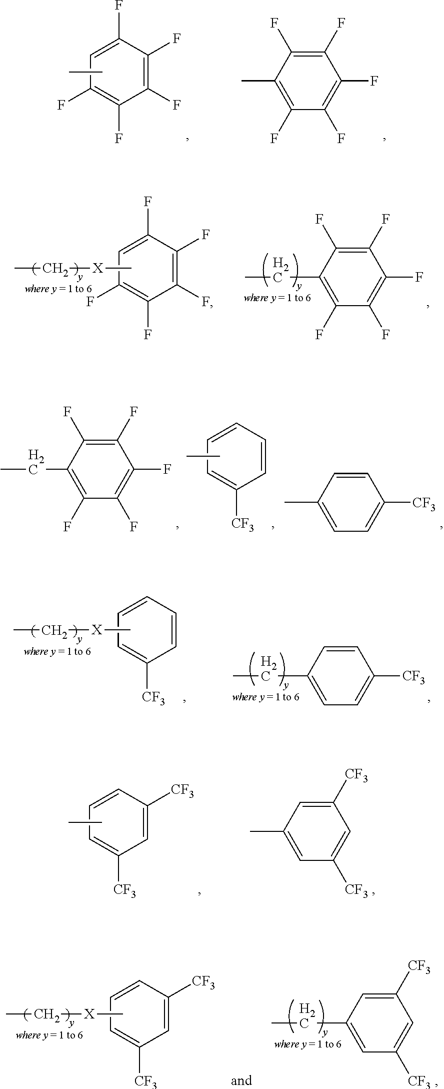

wherein X is any suitable linker molecule and y is an integer value, typically between 1 and 6.

The present inventors have unexpectedly found that hydrophobic blocks (H) comprising aminoaryl or aminoheteroaryl groups lead to improved manufacturing and solubility in polar aprotic solvents and alcohols. Therefore, in certain preferred embodiments, the hydrophobic block (H) comprises moieties of the formula —Ar—NHR, where Ar can be a aryl or heteroaryl, and R is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl. Non-limiting examples of aminoaryl or aminoheteroaryl groups include but are not limited to:

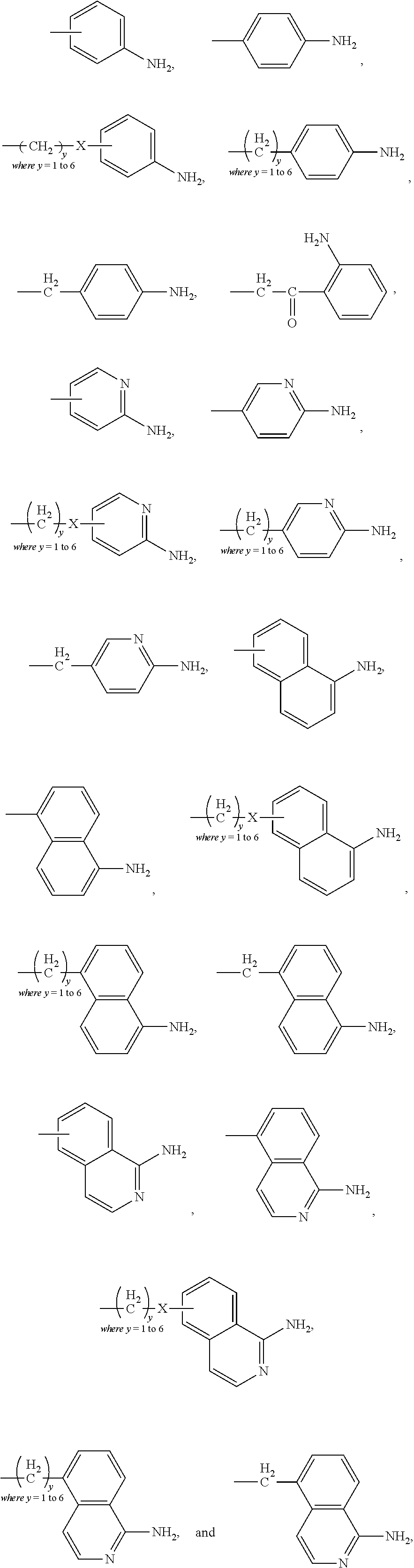

wherein X is any suitable linker molecule and y is an integer value, typically between 1 and 6.

In some embodiments, the hydrophobic block (H) comprises polymers (or oligomers) that further comprise hydrophobic monomers with fused aryl groups (e.g., naphthyl) or fused heteroaryl groups (e.g., xanthenyl or quinolinyl). In some embodiments, the hydrophobic block (H) comprises reactive monomers linked to hydrophobic drug molecules. In some embodiments, the hydrophobic drug molecules (e.g., imidazoquinolines) are aromatic and thus the reactive monomers linked to hydrophobic drug molecules comprising aromatic groups may also be described as hydrophobic monomers comprising aromatic groups or reactive monomers linked to drugs.

In some embodiments, the hydrophobic block (H) comprises a poly(amino acid) of Formula I:

(M)ₘ-(N)ₙ—(O)ₒ—(P)ₚ—R³

wherein the poly(amino acid) of Formula I comprises monomers selected from hydrophobic amino acids (M), reactive amino acids (N), spacer amino acids (O), charged amino acids (P) and combinations thereof provided that at least monomer M or N are present; m, n, o and p denote that there are an integer of repeat units of monomers M, N, O and P, respectively, which may be distributed along the polymer in a specific or random order; and R₃ is typically selected from hydrogen, NH₂, NH₂—CH₃, NH₂—(CH₂)_{y5}CH₃, OH, or drug molecules (D) either linked directly or through X1.

In some embodiments, P is absent. In other embodiments, N, O, and P are each absent.

In some embodiments, P is

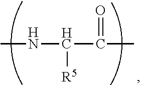

wherein each R⁵, independently, is a group that comprises 1 to 2 charged functional groups.

In some embodiments, O is

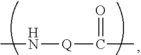

wherein each Q, independently, is selected from (CH₂)_{y6} and (CH₂CH₂O)_{y7}CH₂CH₂; each y6 is independently selected from an integer from 1 to 6; and each y7 is independently selected from an integer from 1 to 4.

In some embodiments, N is

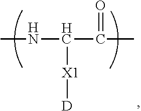

wherein each X1, independently, is a suitable linker; and each D, independently, is a drug molecule.

In some embodiments, M is

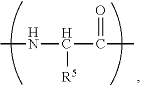

wherein each R⁴ is, independently, a hydrophobic group.

In some embodiments, the hydrophobic block (H) comprises a poly(amino acid) of Formula I:

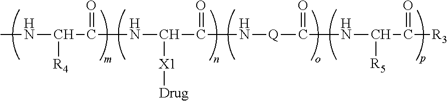

wherein the poly(amino acid) of Formula I comprises monomers selected from hydrophobic amino acids (M), reactive amino acids (N), spacer amino acids (O), charged amino acids (P) and combinations thereof provided that at least monomer M or N are present; m, n, o and p denote that there are an integer of repeat units of monomers M, N, O and P, respectively, which may be distributed along the polymer in a specific or random order; R³ is typically selected from hydrogen, NH₂, NH₂—CH₃, NH₂—(CH₂)_{y5}CH₃, OH, or drug molecules (D) either linked directly or through X1; R⁴ is any hydrophobic group typically selected from aryl or heteroaryl groups; R⁵ is any group that comprises one or more functional groups that are charged in aqueous solutions or are pH-responsive and charged in aqueous solutions at certain pH ranges; Q is typically selected from any lower alkyl or heteroalkyl including but not limited to (CH₂)_{y6} and (CH₂CH₂O)_{y7}CH₂CH₂, where y6 is any integer from 1 to 6 and y7 is an integer typically selected from 1 to 4; and, the N-terminus is linked to either (i) a solubilizing block (S) directly or indirectly via a spacer (B) and/or a Linker U; (ii) a peptide antigen (A) either directly or indirectly via an extension (E1 or E2) and/or Linker U; or (iii) a drug molecule either directly or via X1. Note: hydrophobic amino acids, reactive amino acids, spacer amino acids and charged amino acids are sometimes described more generally as hydrophobic monomers, reactive monomers, spacer monomers and charged monomers, respectively.

In preferred embodiments of poly(amino acids) of Formula I, R⁴ is

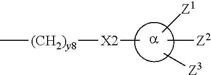

wherein,
α is aryl or heteroaryl;
X2 is present or absent and when present is is a suitable linker;
y8 is selected from an integer from 0 and 6; and
Z¹, Z², and Z³ are each independently selected from H, F, hydroxy, amino, alkyl, and fluoroalkyl.

In preferred embodiments of poly(amino acids) of Formula I, a is aryl, e.g., phenyl or naphthyl. In other embodiments, a is heteroaryl, e.g., imidazolyl, pyridinyl, quinolinyl, isoquinolinyl, indolyl, and benzimidazolyl.

In preferred embodiments of poly(amino acids) of Formula I, X2 is absent. In other embodiments, X2 is present and is selected from C(O), CO₂(CH₂)_{y9}, and C(O)NH(CH₂)_{y9}, NHC(O) and NHC(O)(CH₂)_{y9}, wherein y9 is an integer typically selected from 1 to 6. In other embodiments, X2 is present and is selected from lower alkyl and PEG groups.

In preferred embodiments of poly(amino acids) of Formula I, the poly(amino acid) of Formula I comprises hydrophobic amino acids, M, selected from any natural or non-natural amino acid that comprises a hydrophobic group, R⁴. In preferred embodiments, R⁴ is selected from hydrophobic groups comprising aryl groups, heteroaryl groups, aminoaryl, and/or aminoheteroaryl. Non-limiting examples of R⁴ include but are not limited to:

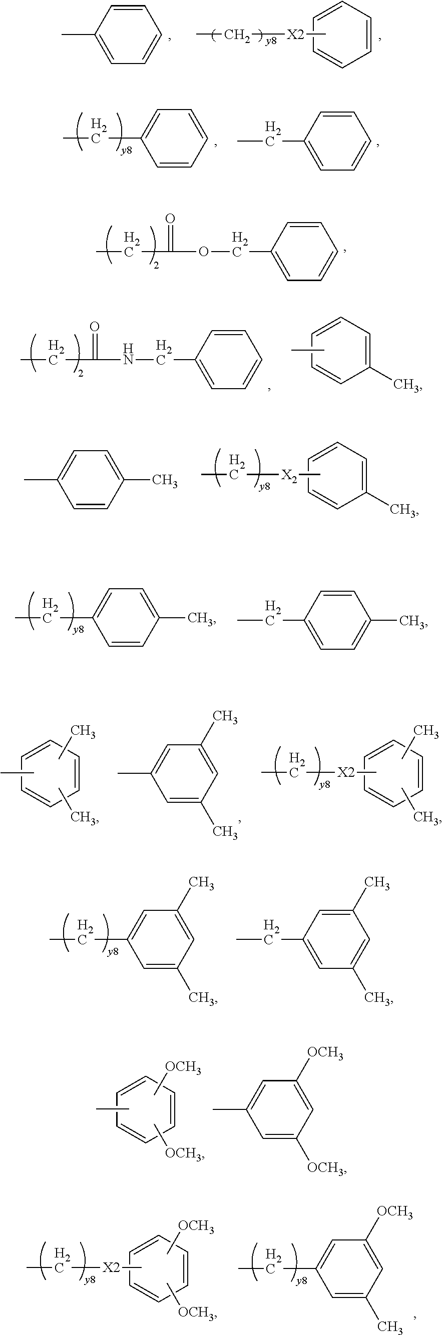

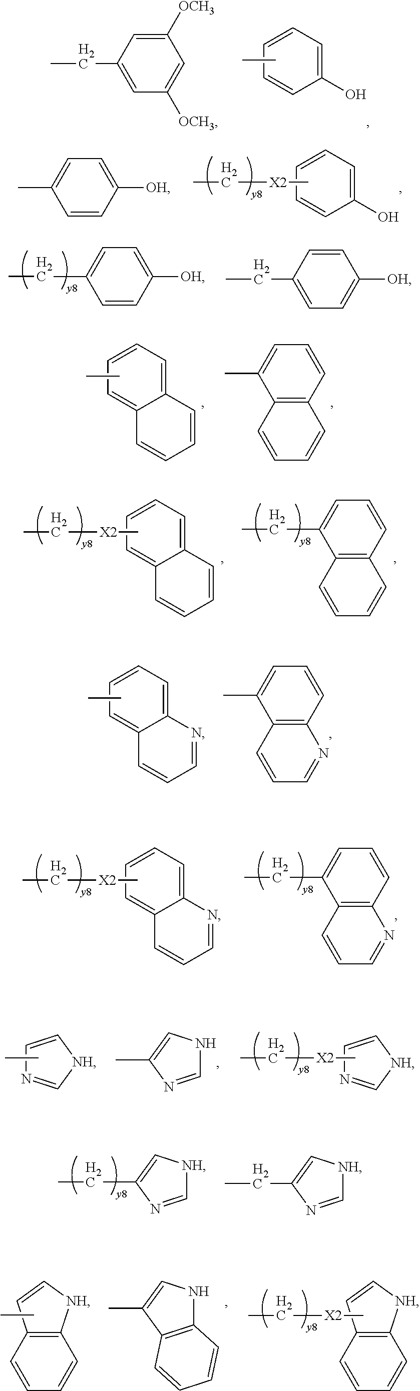

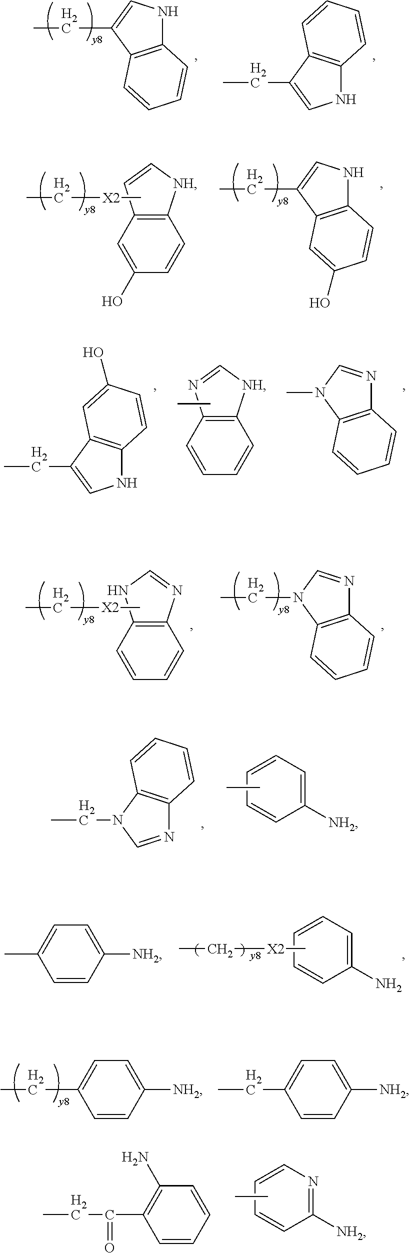

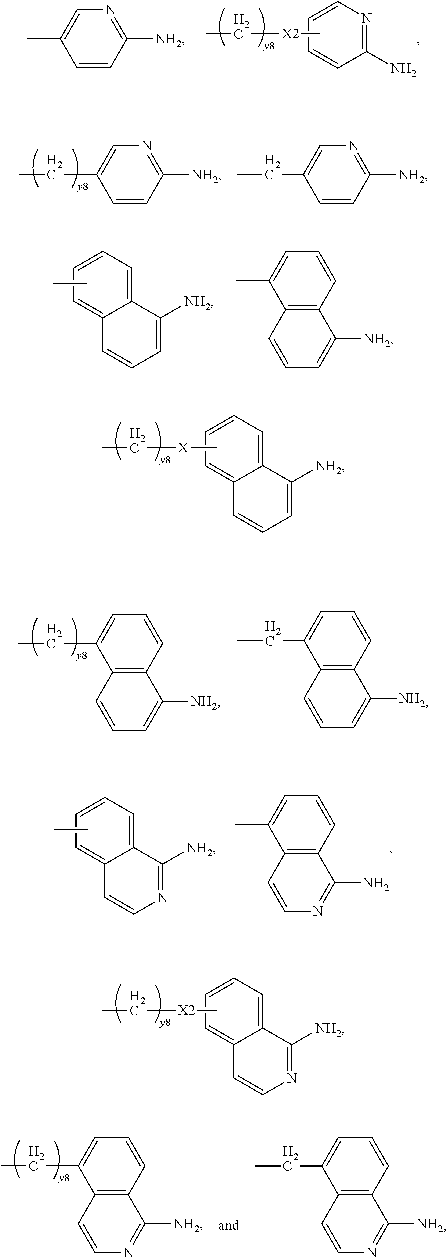

wherein X2 is any suitable linker molecule and y8 is an integer value, typically between 0 and 6. In preferred embodiments y8 is 1.

In non-limiting examples, wherein R⁴ is

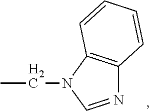

monomer M is:

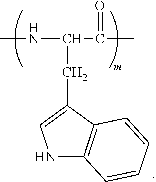

In some embodiments, the poly(amino acid)-based hydrophobic block (H) of Formula I comprises reactive amino acids, N, that are selected from any natural or non-natural amino acid, wherein a drug molecule (D) is linked directly or through X1 to the monomer. Suitable reactive amino acids include but are not limited to any amino acids bearing a group suitable for attachment of drug molecules, include amino acids with azide, alkyne, tetrazine, transcyclooctyne (TCO), protected hydrazine, ketone, aldehyde, certain hydroxyl groups, isocyanate, isothiocyanate, carboxylic acids, activated carboxylic acids, activated carbamates, activated carbamates, protected maleimide, thiol and/or amine groups.

X1 is any suitable linker for linking drug molecules, D, to the hydropbobic block (H), including to the reactive amino acid, N, of poly(amino acids) and is typically selected from —(CH₂)_{y10}-FG3 and —(CH₂)_{y10}-R⁶ (or —C(O)—(CH₂)_{y10}-FG3 and —C(O)—(CH₂)_{y10}-R⁶ when drugs are linked at the N-terminus or off of amine groups, or —NH—(CH₂)_{y10}-FG3 and —NH—(CH₂)_{y10}-R⁶ when drugs are linked at the C-terminus or off of carbonyl groups), wherein y10 is any integer, typically selected from 1 to 6, and R⁶ is typically selected from any one or more of —C(O)—NH—R⁷, —NH—C(O)—R⁷, —NH—C(O)—O—R⁷, —O—C(O)—NH—R⁷, —O—C(O)—R⁷, —C(O)—O—R⁷, —O—R⁷, O—C(O)—W, or —C(O)—W, wherein R⁷ is typically selected from any one or more of —(CH₂)_{y11}—W, —(CH₂)_{y1}—(OCH₂CH₂)_{y12}—W, —(CH₂)_{y11}—(OCH₂CH₂)_{y12}—(CH₂)_{y13}—W, —CHR⁸—C(O)—W, —CHR⁸—C(O)—(NH—CHR⁸—C(O))ⱼ—W, —(CH₂)_{y11}—C(O)—NH—CHR⁸—C(O)—W, —(CH₂)_{y1}, —C(O)—NH—CHR⁸—C(O)—(NH—CHR⁸—C(O))ⱼ—W, —(CH₂)_{y11}—(OCH₂CH₂)_{y12}—C(O)—NH—CHR⁸—C(O)—W, —(CH₂)_{y11}—(OCH₂CH₂)_{y12}—(CH₂)_{y13}C(O)—NH—CHR⁸—C(O)—W, —(CH₂)_{y11}—(OCH₂CH₂)_{y12}—C(O)—NH—CHR⁸—C(O)—(NH—CHR⁸—C(O))ⱼ—W, —(CH₂)_{y11}—(OCH₂CH₂)_{y12}—(CH₂)_{y13}—C(O)—NH—CHR⁸—C(O)—(NH—CHR⁸—C(O))ⱼ—W, —CHR⁸—C(O)—NH—C₆H₄—CH₂—O—C(O)—W, —CHR⁸—C(O)—NH(CH₃)(CH₂)₂—O—C(O)—W, —CHR⁸—C(O)—(NH—CHR⁸—C(O))ⱼ—NH—C₆H₄—CH₂—O—C(O)—W, —CHR⁸—C(O)—(NH—CHR⁸—C(O))ⱼ—NH(CH₃)(CH₂)₂—O—C(O)—W, —(CH₂)_{y11}—C(O)—(NH—CHR⁸—C(O))ⱼ—NH—C₆H₄—CH₂—O—C(O)—W, —(CH₂)_{y11}—C(O)—(NH—CHR⁸—C(O))ⱼ—NH(CH₃)(CH₂)₂—O—C(O)—W, —(CH₂)_{y11}—(OCH₂CH₂)_{y12}—C(O)—(NH—CHR⁸—C(O))ⱼ—NH—C₆H₄—CH₂—O—C(O)—W, —(CH₂)_{y11}—(OCH₂CH₂)_{y12}—C(O)—(NH—CHR—C(O))ⱼ—NH(CH₃)(CH₂)₂—O—C(O)—W, —(CH₂)_{y11}—(OCH₂CH₂)_{y12}—(CH₂)_{y13}C(O)—(NH—CHR⁸—C(O))ⱼ—NH—C₆H₄—CH₂—O—C(O)—W, —(CH₂)_{y11}—(OCH₂CH₂)_{y12}—(CH₂)_{y13}C(O)—(NH—CHR⁸—C(O))ⱼ—NH(CH₃)(CH₂)₂—O—C(O)—W, —(CH₂)_{y1}—(OCH₂CH₂)_{y12}—(CH₂)_{y13}—C(O)—NH—(CH₂)_{y14}—C(O)—(NH—CHR⁸—C(O))ⱼ—NH—C₆H₄—CH₂—O—C(O)—W, —(CH₂)_{y11}—(OCH₂CH₂CH₂)_{y13}C(O)—NH—(CH₂)_{y14}—C(O)—(NH—CHR⁸—C(O))ⱼ—NH(CH₃)(CH₂)₂—O—C(O)—W, —(CH₂)_{y11}—(OCH₂CH₂)_{y12}—C(O)—NH—(CH₂)_{y14}—C(O)—(NH—CHR⁸—C(O))ⱼ—NH-C₆H₄—CH₂—O—C(O)—W, —(CH₂)_{y11}—(OCH₂CH₂)_{y12}—C(O)—NH—(CH₂)_{y14}—C(O)—(NH—CHR⁸—C(O))ⱼ—NH(CH₃)(CH₂)₂—O—C(O)—W, —CHR⁸—C(O)—NH—(CH₂)_{y15}—W, —CHR⁸—NH—C(O)—(CH₂)_{y15}—W, —CHR⁸—C(O)—(NH—CHR⁸—C(O))ⱼ—NH—(CH₂)_{y15}—W, —CHR⁸—NH—(C(O)—CHR⁸—NH)ⱼ—C(O)—(CH₂)_{y15}—W, where y11, y12, y13, y14, y15 and j are each independently selected from any integer typically selected from 1 to 6, R⁸ is any amino acid side group, and W can be independently selected from H (hydrogen), FG3, LG and w; wherein FG3 is any suitable functional group for attachment to the drug molecule, which may be selected from, but not limited to, carboxylic acid, activated carboxylic acids (e.g., carbonylthiazolidine-2-thione (“TT”), NHS or nitrophenol esters), carboxylic acid anhydrides, amine and protected amines (e.g., tert-butyloxycarbonyl protected amine), OSi(CH₃), alkene, azide, alkyne, stained-alkyne, halogen (e.g., fluoride, chloride), olefins and endo cyclic olefins (e.g., allyl), CN, OH, and epoxy, hydrazines (including hydrazides), carbohydrazides, aldehydes, ketones, carbamates and activated carbamates, LG is any suitable leaving group, which may be selected from, but not limited to any suitable leaving group (e.g., NHS, TT, nitrophenol, etc.), and, w is a group that results from either the reaction of FG4 with FG3 or the displacement of LG with FG4, and is typically selected from NH—, C(O)—, NH—C(O)—, C(O)—NH—, O—C(O)—NH—, C(O)—NH—N═C(CH₃)—, NH—N═C(CH₃)— or —C(CH₃)═N—NH—C(O)—, wherein w is always linked to D either directly (i.e., w-D) or indirectly via X3 (i.e., w-X3-D).

Drug molecules (D) may be attached to the reactive amino acid, N, directly or via X1 through reaction of FG4 with FG3, wherein FG4 is any suitable functional group on the drug (D) that is reactive with FG3. Alternatively, drug molecules (D) may be linked to the reactive amino acid, N, via X1 through displacement of LG with any suitable FG4 comprising a nucleophile, e.g., a primary amine, or drug molecules (D) may be linked to the reactive amino acid, N, via X1 through displacement of an LG present on the drug molecule with any suitable FG3 comprising a nucleophile.

In preferred embodiments, FG3 is a carboxylic acid and FG4 is an amine, which react to form an amide. In non-limiting examples, X1 is selected from —(CH₂)_{y10}-FG3, y10 is 2, FG3 is a carboxylic acid, and FG4 present on the drug is an amine (i.e., NH₂-D), which react to form an amide, which may be represented as —(CH₂)₂—C(O)-D (amine not shown) or —(CH₂)₂—C(O)—NH-D (amine shown), indicating that the drug is linked via an amide bond at the carbonyl of X1, which (after amide bond formation) may be described as —(CH₂)_{y10}-R⁶, wherein y10 is 2, R⁶═C(O)—W, and W is the group w, which is NH— and is linked to D to give —(CH₂)₂—C(O)—NH-D.

The drug may additionally comprise a linker, X3, between the reactive functional group FG4 and the pharmacophore, e.g., FG4-X3-D. Specific, preferred compositions of X3 are described elsewhere.

In other embodiments, FG3 is an amine and FG4 is a carboxylic acid, which react to form an amide. In non-limiting examples, X1 is —(CH₂)_{y10}-FG3, y10 is 4, FG3 is an amine, and FG4 present on the drug is a carboxylic acid (i.e., COOH-D), which react to form an amide, which may be represented as —(CH₂)₄—NH-D (carbonyl not shown) or —(CH₂)₄—NH—C(O)-D (carbonyl shown), indicating that the drug is linked via an amide bond at the amine of X1.

In still other embodiments, FG3 is a ketone or aldehyde and FG4 is a hydrazide or carbohydrazide, which react to form a hydrazone. In non-limiting examples, X1 is —(CH₂)_{y10}-R⁶, y10 is 4, R⁶ is —NH—C(O)—R⁷, R⁷ is (CH₂)_{y11}—W, y11 is 2 and W is C(O)—CH₃, and FG4 present on the drug molecule is a hydrazide (NH₂—NH₂—C(O)-D), which reacts with X1, i.e., —(CH₂)₄—NH—C(O)—(CH₂)₂—C(O)—CH₃ to form a hydrazone bond, i.e., —(CH₂)₄—NH—C(O)—(CH₂)₂—C(CH₃)═N—NH—C(O)-D. In still other embodiments, FG3 is a hydrazide or carbohydrazide and FG4 is a ketone or aldehyde that reacts to form a hydrazone. In non-limiting examples, X1 is —(CH₂)_{y10}-R⁶, y10 is 2, R⁶ is —C(O)—W, W is FG3 and FG3 is —NH—NH₂ and FG4 present on the drug molecule is a ketone CH₃C(O)-D (or optionally CH₃C(O)—X3-D), which reacts with X1 to form —(CH₂)₄—C(O)—NH—NH₂ to form a hydrazone bind, i.e., form —(CH₂)₄—C(O)—NH—N═C(CH₃)-D.

In certain preferred compositions, drug molecules (D) are linked directly to the reactive amino acid, N. A non-limiting example of a reactive amino acid comprising a linker selected from —(CH₂)_{y10}-FG3, wherein y10=2, FG3 is carboxylic acid (i.e., the reactive amino acid is glutamic acid) linked to a drug molecule is shown below for clarity:

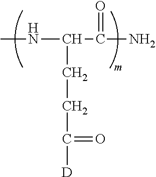

In certain other preferred embodiments, drug molecules (D) are linked to the reactive amino acid (N) via an enzyme degradable peptide and/or self-immolative linker, wherein the self-immolative linker is typically selected from —NH—C₆H₄—CH₂—O—C(O)— or —NH(CH₃)(CH₂)₂—O—C(O)— and FG4 present on the drug is an amine, e.g., NH₂-D or NH₂—X3-D, which results in a carbamate bond between the linker and the drug. In non-limiting examples, the reactive monomer comprises a linker selected from (CH₂)_{y10}-R⁶, wherein y10=2, R⁶ is —C(O)—NH—R⁷ and R⁷ is (CH₂)_{y11}—C(O)—(NH—CHR⁸—C(O))ⱼ—NH—C₆H₄—CH₂—O—C(O)—W, wherein y11 is 2, R⁸ is any amino acid group, j is an integer typically selected from 1 to 6, W is selected from the group w, which is NH-linked to the drug (D), as shown here:

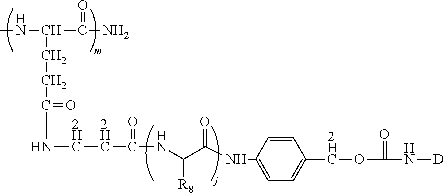

In preferred compositions of X1 comprising enzyme degradable linkers, the enzyme degradable linker typically comprises between 1 and 6 amino acids, such as 1, 2, 3, 4, 5 or 6 amino acids selected from single amino acids, dipeptides, tripeptides, tetrapeptides, pentapeptides and hexapeptides recognized and cleaved by enzymes, such as cathepsins and/or the immunoproteasome.

Reactive amino acids (N) may comprise functional groups that can impart charge; however, the classification of an amino acid as a reactive amino acid monomer is context-dependent and based on its intended use. For example, monomers comprising carboxylic acids may be referred to as charged monomers if the carboxylic acid is not used for drug attachment, whereas the same monomers linked to an amine bearing drug molecule, e.g., via an amide bind, would be considered a reactive monomer.

In some embodiments, the poly(amino acid)-based polymer of Formula I comprises spacer amino acids, O, that are selected from any natural or non-natural amino acid that are non-bulky and near neutral, such as a PEG amino acid spacer, e.g., Q of monomer O is a lower alkyl or PEG, e.g., —(CH₂)_{y6}-, —CH₂—CH₂—O- or —(CH₂—CH₂-0)_{y7}CH₂—CH₂—, wherein y6 and y7 are each independently an integer typically between 1 and 6. Alternatively, monomer 0, is selected from amino acids with a small, i.e., non-bulky, substituent selected from hydrogen, lower alkyl or a lower alkyl comprising a hydroxyl and is provided to increase the spacing or flexibility of the polymer backbone.

Non-limiting examples include:

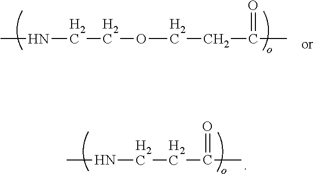

In some embodiments, the poly(amino acid)-based polymer of Formula I comprises optional co-monomer(s), P, that are selected from any natural or non-natural amino acid, wherein R₅ is selected from any group comprising a functional group that carries charge either permanently or at a specific pH in aqueous solutions. Non-limiting examples of charged amino acids include any natural or non-natural amino acid that comprise amine, quaternary ammonium, sulfonic acid, sulfuric acid, sulfonium, phosphoric acid, phosphonic acid, phosphonium, carboxylic acid, boronic acid functional groups and/or combination thereof, including zwitterions, which may be linked either directly or via a suitable linker molecule, as well as any composition of salts thereof. Non-limiting examples of salts include, e.g., positively charged functional groups, e.g., ammonium ions paired with halide (e.g., chloride) ions. Other non-limiting examples of suitable salts of charged amino acids include conjugate bases of carboxylic, sulfonic and phosphonic acids, paired with group 1 metals, such as sodium, or ammonium or guanidinium ions.

In some preferred embodiments of amphiphiles for nucleic acid delivery, the amphiphile comprises a hydrophobic block (H) further comprising a poly(amino acid)-based polymer of Formula I that includes R⁵ selected from groups that have net positive charge, which include but are not limited to:

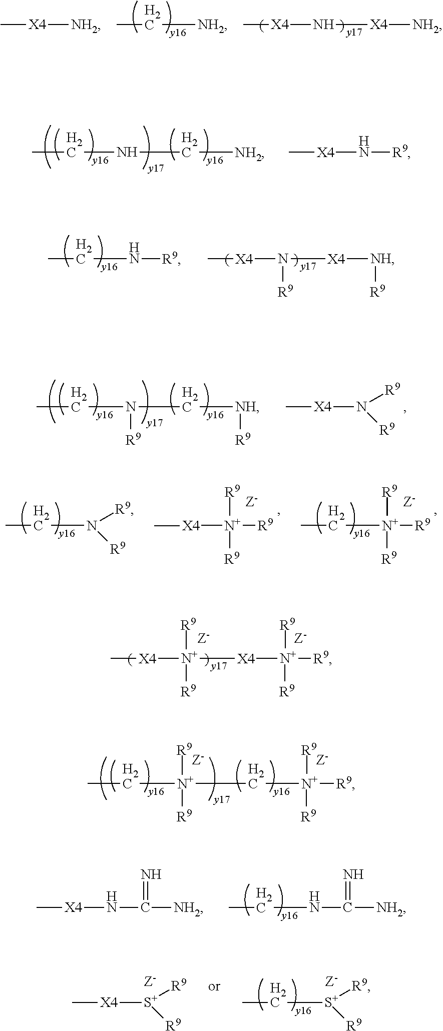

wherein X4 is any suitable linker, y16 and y17 are each independently any integer, typically selected from between 1 to 6, R⁹ is selected from lower alkyl or branched alkyl groups, such as CH₃, CH₂CH₃, CH₂CH₂CH₃, CH(CH₃)₂, H₂CH(CH₃)₂ or the like, and Z⁻ is any suitable counter anion, which is typically selected from conjugate bases of weak acids or halide ions, such as Cl⁻, I⁻, or Br⁻.

The hydrophobic block (H) functions to drive particle assembly in aqueous solutions and therefore, in preferred embodiments of amphiphiles, peptide antigen conjugate or drug molecule conjugates, the hydrophobic block (H) comprises hydrophobic amino acids and/or reactive amino acids linked to hydrophobic drug molecules. In preferred embodiments of poly(amino acid)-based polymers of Formula I, the poly(amino acid)-based polymer (or oligomer) of Formula I comprises hydrophobic amino acids (M) and/or reactive amino acids (N) linked to hydrophobic drug molecules, and optionally spacer amino acids (O) and/or charged amino acids (P). In preferred embodiments of amphiphiles, peptide antigen conjugate or drug molecule conjugates used for peptide antigen delivery and/or for the delivery of neutral drug molecules, the hydrophobic block (H) is typically selected from poly(amino acid)-based polymers of Formula I comprising hydrophobic amino acids (M) and/or reactive amino acids (N) linked to hydrophobic drug molecules, and optionally spacer amino acids (O), but not charged amino acids (P). In contrast, wherein the amphiphiles, peptide antigen conjugate or drug molecule conjugates are used for nucleic acid delivery or for the delivery of charged drug molecules, the hydrophobic block (H) is typically selected from poly(amino acid)-based polymers of Formula I comprising hydrophobic amino acids (M) and/or charged amino acids (P), wherein the charge of the charge amino acid is opposite that of the nucleic acid or charged drug molecule, and optionally reactive amino acids (N) linked to hydrophobic drug molecules and spacer amino acids (O). Particular compositions of hydrophobic blocks (H) based on poly(amino acid)-based polymers or oligomers of Formula I that led to unexpected improvements in biological activity are described throughout the specification.

In some embodiments, the hydrophobic block (H) is a poly(amino acid) of Formula I comprising entirely hydrophobic monomers (m):

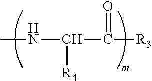

Non-limiting examples include:

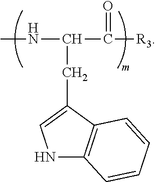

A non-limiting example of a poly(amino acid) of Formula I composed entirely of hydrophobic monomers (M) selected from tryptophan, wherein m is equal to 5 (i.e., 5 monomeric units), R³ is an amine and the N-terminal amine is linked to a solubilizing block (S) either directly or indirectly through a spacer (B) and/or linker U, is shown here for clarity:

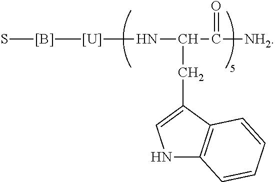

In some embodiments drug molecules (D) are linked via the N-terminus or C-terminus of hydrophobic blocks (H) comprising poly(amino acids) of Formula I. A non-limiting example is shown here for clarity:

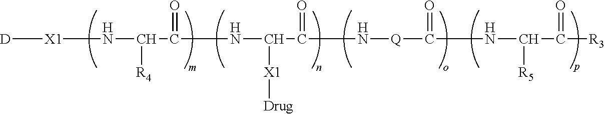

Wherein the poly(amino acid) comprises hydrophobic amino acids selected from tryptophan and R³ is NH₂ the structure is:

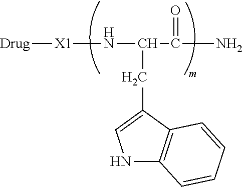

Wherein when X1 comprises a PAB-Cit-Val linked to the poly(amino acid) via a succinate linker the structure is:

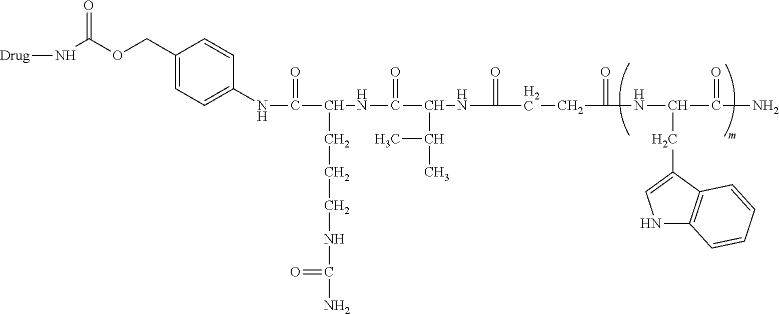

Alternatively, wherein X1, comprises a PAB-Cit-Val linked to the poly(amino acid) via Linker U resulting from the reaction between azide and DBCO, an exemplary strained alkyne, wherein the DBCO moiety is linked to poly(amino acid) via Ahx, the structure is:

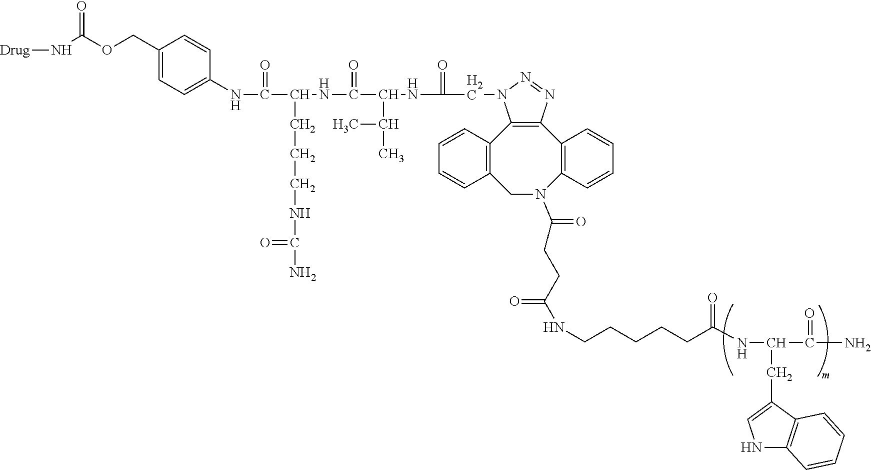

Herein, we report the unexpected finding that amphiphilic copolymers with hydrophobic polymers or oligomers (H) which comprise poly(amino acid)-based copolymers that include aromatic amino acids (e.g., phenylalanine, amino phenylalanine, histidine, tryptophan, tyrosine, benzyl glutamate) and/or aromatic drug molecules (e.g., imidazoquinolines), have unexpected improvements in manufacturability through improved solubility in polar aprotic solvents and alcohols as well as improved particle stability as compared with poly(amino acids) comprising hydrophobic amino acids selected from aliphatic amino acids.

An additional notable finding relates to how the number of monomer units comprising the hydrophobic block (H) impacts particle formation. For example, poly(amino acid)-based hydrophobic blocks (H) which comprise at least 5 hydrophobic amino acids were typically needed to ensure stable assembly of particles comprising amphiphiles of formula S-[B]-[U]-H (optionally further comprising a drug molecule, e.g., S-[B]-[U]-HD). Though, unexpectedly, poly(amino acid)-based hydrophobic blocks (H) which comprise oligomers with as few as 3 monomers that included aromatic rings were found to be sufficient to drive stable particle assembly. Notably, increasing the number of hydrophobic monomers comprising the poly(amino acid)-based hydrophobic block (H) from 3 to 5 and from 5 to 10 hydrophobic monomers led to improved particle assembly. While increasing the total number of monomers comprising hydrophobic blocks (H) (i.e. total number of monomers or degree of polymerization) led to improved particle stability, both the total number of monomers and composition of the poly(amino acids) of Formula I also impacted manufacturability as well as stability. For example, poly(amino acids) of Formula I comprising between 10-30 consecutive monomers selected from hydrophobic amino acids comprising aryl groups and/or heteroaryl groups were more reliably manufactured than poly(amino acids) of Formula I comprising between 10-30 consecutive monomers selected from hydrophobic amino acids comprising aliphatic groups.

Therefore, in preferred embodiments of poly(amino acid)-based hydrophobic blocks (H), the hydrophobic block (H) comprises 3 or more, preferably about 3 to about 100 hydrophobic amino acids (M) and/or reactive amino acids linked to drug molecules (D), though, more preferably between about 3 to 30 hydrophobic amino acids (M) and/or reactive amino acids linked to drug molecules (D), more preferably wherein the hydrophobic amino acids and/or reactive amino acids linked to drug molecules (D) further comprise aryl groups, heteroaryl, aminoaryl and/or aminoheteroaryl.

Hydrophobic Blocks (H) with Branched Architecture

In some embodiments, the amphiphilic block copolymer comprises a hydrophobic block (H) that is branched. In certain preferred embodiments, the hydrophobic block (H) comprises a dendron, wherein the focal point is linked to either (i) a solubilizing block (S) either directly or indirectly via a spacer (B) and/or Linker U, (ii) an antigen (A) either directly or indirectly via an extension (E1 or E2) and/or Linker U; (iii) a drug molecule either directly or via a Linker U; or, (iv) a capping group, and the terminal functional groups (FGt) are linked to hydrophobic molecules, e.g., hydrophobic drug molecules, more preferably hydrophobic molecules comprising aromatic groups, e.g., hydrophobic drug molecules comprising aromatic groups.

Non-limiting examples of amphiphiles, peptide antigen conjugates or drug molecule conjugates comprising hydrophobic blocks (H) with dendron architecture, wherein the terminal functional groups (FGt) are linked to hydrophobic drug molecules are provided below for clarity:

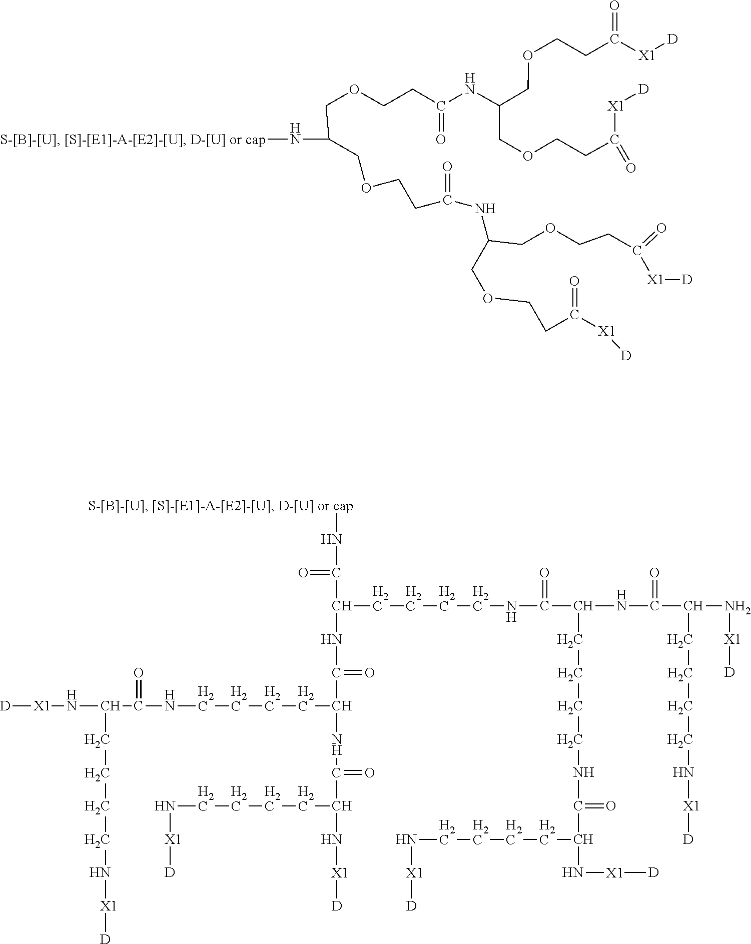

Wherein X1 is either present or absent and when present is any suitable linker and D is any suitable drug molecule, preferably selected from hydrophobic drug molecules comprising aromatic groups, and the focal point is attached to either (i) a solubilizing block (S) either directly or indirectly via a spacer (B) and/or Linker U, (ii) an antigen (A) either directly or indirectly via an extension (E1 or E2) and/or Linker U; (iii) a drug molecule either directly or via a Linker U; or, (iv) a capping group.

Additional examples of hydrophobic blocks (H) with dendron architecture that have particular utility for certain applications and/or lead to unexpected improvements in manufacturing and/or biological activity are provided throughout the specification.

Density (Mol %) of Hydrophobic Groups and/or Drug Molecules

The density (i.e., mol %) of the hydrophobic monomers (e.g., hydrophobic amino acids or reactive monomers linked to hydrophobic drug molecules) incorporated into polymer-based hydrophobic blocks (H), e.g., poly(amino acids) of Formula I, were found by the inventors of the present disclosure to have a major impact on particle stability and biological activity. Thus, the density (i.e., mol %) of hydrophobic monomers (e.g., hydrophobic amino acids or reactive monomers linked to hydrophobic drug molecules) incorporated into polymer-based hydrophobic blocks should be carefully selected. In general, the density (mol %) of hydrophobic monomers (e.g., hydrophobic amino acids or reactive monomers linked to hydrophobic drug molecules) required is inversely proportional to the length (i.e. degree of polymerization) of the polymer.

For instance, the preferred density (mol %) of hydrophobic monomers (e.g., hydrophobic amino acids, M) and/or reactive monomers linked to hydrophobic drug molecules (e.g., reactive amino acids (N) linked to hydrophobic drug molecules) is typically 100 mol % for polymers (or “oligomers”) with 3 monomers; 75-100 mol % for polymers (or “oligomers”) with 4 monomers, such as 75 mol % or 100 mol % for polymers with 4 monomers; 60-100 mol % for polymers (or “oligomers”) with 5 monomers, such as 60 mol %, 80 mol % or 100 mol %; 50-100 mol % for polymers (or “oligomers”) with 6 monomers, such as 50 mol %, 66.6 mol %, 83.3 mol % and 100 mol %; 42-100 mol % for polymers (or “oligomers”) with 7 monomers, such as 42 mol %, 57 mol %, 71 mol %, 85.7 mol % and 100 mol %; 37.5-100 mol % for polymers (or “oligomers”) with 8 monomers, such as 37.5 mol %, 50 mol %, 75 mol %, 87.5 mol % and 100 mol %; 33.3-100 mol % for polymers (or “oligomers”) with 9 monomers, such as 33.3 mol %, 44.4 mol %, 55.6 mol %, 66.6 mol %, 77.9 mol %, 88.9 mol % and 100 mol %; 30-100 mol % for polymers (or “oligomers”) with 10 monomers, such as 30 mol %, 40 mol %, 50 mol %, 60 mol %, 70 mol %, 80 mol %, 90 mol % and 100 mol %. The preferred density (mol %) of hydrophobic monomers (e.g., hydrophobic amino acids, M) and/or reactive monomers linked to hydrophobic drug molecules (e.g., reactive amino acids (N) linked to hydrophobic drug molecules) for polymers with between 11 and 20 monomers is typically between 20 mol % to 100 mol %, such as 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, 55 mol %, 56 mol %, 57 mol %, 58 mol %, 59 mol %, 60 mol %, 61 mol %, 62 mol %, 63 mol %, 64 mol %, 65 mol %, 66 mol %, 67 mol %, 68 mol %, 69 mol %, 70 mol %, 71 mol %, 72 mol %, 73 mol %, 74 mol %, 75 mol %, 76 mol %, 77 mol %, 78 mol %, 79 mol %, 80 mol %, 81 mol %, 82 mol %, 83 mol %, 84 mol %, 85 mol %, 86 mol %, 87 mol %, 88 mol %, 89 mol %, 90 mol %, 91 mol %, 92 mol %, 93 mol %, 94 mol %, 95 mol %, 96 mol %, 97 mol %, 98 mol %, 99 mol % or 100 mol %, provided that at least 3 hydrophobic monomers (M) or reactive monomers (N) linked to hydrophobic drugs are present; 10-100 mol %, more preferably 20-80 mol %, such as 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, 55 mol %, 56 mol %, 57 mol %, 58 mol %, 59 mol %, 60 mol %, 61 mol %, 62 mol %, 63 mol %, 64 mol %, 65 mol %, 66 mol %, 67 mol %, 68 mol %, 69 mol %, 70 mol %, 71 mol %, 72 mol %, 73 mol %, 74 mol %, 75 mol %, 76 mol %, 77 mol %, 78 mol %, 79 mol % or 80 mol % for polymers with between 21 and 30 monomers, provided that at least 3 hydrophobic monomers (M) or reactive monomers (N) linked to hydrophobic drugs are present; and, 5-60 mol %, more preferably, 10-40 mol % for polymers with >30 monomers, such as 10 mol %, 11 mol %, 12, mol %, 13 mol %, 14 mol %, 15 mol %, 16 mol %, 17 mol %, 18 mol %, 19 mol %, 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol % and 40 mol % for polymers with >30 monomers.

In the above examples, in preferred embodiments, the polymer is a poly(amino acid) and the monomer is selected from hydrophobic monomers (e.g., hydrophobic amino acid and/or reactive monomers linked to hydrophobic drug molecules) that comprise an aryl group, and, more preferably, a heteroaryl, aminoaryl, and/or aminoheteroaryl. Additionally, in the above examples, the hydrophobic monomer may be selected from two or more monomers, e.g., two or more distinct hydrophobic monomers (e.g., hydrophobic amino acids), or one or more hydrophobic monomers and one or more reactive monomers (e.g., reactive amino acids) linked to hydrophobic drugs, such that the total mol % of hydrophobic monomers falls within the preferred ranges.

### General Properties of Polymer-Based Hydrophobic Blocks (H)

The average molecular weight of polymer-based hydrophobic blocks (H) can be readily estimated based on the number and composition of monomers (e.g., amino acids for poly(amino acids) and is typically between about 500 g/mol to about 20,000 g/mol. In some embodiments, the polymer molecular weight is between about 1,000 and 5,000, or between about 5,000 and 10,000, or between about 10,000 and 20,000 g/mol.

The polydispersity, Mw/Mn, of the hydrophobic polymer or oligomer (H) typically ranges from about 1.0 to 2.0 and depends on the polymerization technique used. For instance, poly(amino acid)-based hydrophobic polymers or oligomers (H) are typically prepared by solid phase peptide synthesis and will have polydispersity of 1.0 as the polymers are molecularly defined. Polymers formed by chain growth polymerization will have polydispersities>1.0. The hydrophobic polymer or oligomer (H) may also comprise polymers based on cyclic monomers, such as poly(amino acid)-based hydrophobic polymers or oligomers (H) based on amino acid N-carboxyanhydrides (NCAs).

The size of the polymer-based hydrophobic block (H) may either be expressed by the molecular weight or degree of polymerization. For molecularly defined, monodisperse polymers, the length (or degree or degree polymerization) of the polymer can be calculated by dividing the molecular weight (e.g., theoretical or experimentally determined by mass spectrometry) by the average molecular weight of the monomer unit(s) comprising the polymer. For polydisperse polymers, the number-average molecular weight, abbreviated Mn, is preferred for estimating the degree of polymerization. As a non-limiting example, a polydisperse polymer with a Mn of 25 kDa and an average monomer molecular weight of 250 g/mol would have a degree of polymerization of 100. The molecular weight of a polymer can also be calculated by multiplying the degree of polymerization by the average monomer molecular weight.

In preferred embodiments of hydrophobic blocks (H), the molecular weight or Mn, is preferably between about 0.5 kDa and 60 kDa, such as about 0.5 kDa, 1 kDa, 1.5 kDa, 2 kDa, 2.5 kDa, 3 kDa, 3.5 kDa, 4 kDa, 4.5 kDa, 5 kDa, 6 kDa, 7 kDa, 8 kDa, 9 kDa, 10 kDa, 11 kDa, 12 kDa, 13, kDa, 14 kDa, 15 kDa, 16 kDa, 17 kDa, 18 kDa, 19 kDa, 20 kDa, 21 kDa, 22 kDa, 23 kDa, 24 kDa, 25 kDa, 26 kDa, 27 kDa, 28 kDa, 29 kDa, 30 kDa, 31 kDa, 32 kDa, 33 kDa, 34 kDa, 35 kDa, 36 kDa, 37 kDa, 38 kDa, 39 kDa, 40 kDa, 41 kDa, 42 kDa, 43 kDa, 44 kDa, 45 kDa, 46 kDa, 47 kDa, 48 kDa, 49 kDa, 50 kDa, 51 kDa, 52 kDa, 53 kDa, 54 kDa, 55 kDa, 56 kDa, 57 kDa, 58 kDa, 59 kDa or 60 kDa. More preferably, the molecular weight of the hydrophobic block is between about 0.5 kDa to about 20 kDa. In certain embodiments, the hydrophobic block (H) is a poly(amino acid) and has a molecular weight of between about 0.5 kDa and about 10 kDa or about 1.5 kDa to about 5 kDa.

Polymers described herein can be synthesized by any suitable means and should preferably have low or no polydispersity. For instance, poly(amino acids) described herein are typically produced by solid-phase peptide synthesis and are molecularly defined with no polydispersity. Similarly, PEG based spacers and dendrons described herein are produced by controlled processed and have little to no polydispersity. In contrast, polymers produced by radical polymerization will have some degree of polydispersity, which may be calculated by dividing the weight-average molecular weight Mw by Mn, i.e., polydispersity index (PDI)=Mw/Mn. Though, the polydispersity of polymers produced by radical polymerization may be controlled by the polymerization technique utilized. Therefore, in preferred embodiments, living polymerization, e.g., RAFT polymerization, is used to synthesize polymers with PDI less than 2.0, typically between about 1.01 and 1.2.

### Solubilizing Block

The amphiphiles disclosed herein comprise a solubilizing block (S) that functions to impart solubility in aqueous solutions at certain temperature, pH and salt concentration. In certain embodiments, the solubilizing block (S) is soluble in aqueous solutions up to about 1-1,000 mg/mL, e.g., up to about 1 mg/mL, about 10 mg/mL, about 100 mg/mL, about 200 mg/mL, or about 500 mg/mL, though, typically not more than 1,000 mg/mL. In some embodiments, the solubilizing block (S) is soluble in aqueous solutions at certain concentrations, temperatures and/or pH ranges but becomes insoluble or less soluble in response to a change in concentration, temperature and/or pH. Preferred solubilizing blocks (S) are molecules that are soluble at concentrations up to at least 1 mg/mL or up to at least about 10 mg/mL or up to at least about 100 mg/mL at or near physiologic pH (˜pH 7.4), between about pH 6.5 to pH 8.5 or between about pH 6.0 and pH 9.0, and at or near physiologic temperature (˜37° C.), such as between about 32-40° C., and at physiologic salt concentrations (˜10 g/L) and salt composition.

The solubilizing block may be chosen from any molecule that is water soluble and/or has hydrophilic characteristics. In some embodiments the solubilizing block (S) is selected from a linear, branched or brush polymer (or oligomer). The solubilizing block (S) can be a homopolymer or copolymer. The solubilizing block (S) can comprise one or many different types of monomer units. The solubilizing block (S) can be a statistical copolymer or alternating copolymer. The solubilizing block (S) can be a block copolymer, such as the A-B type, or the polymer can comprise a grafted copolymer, whereby two or more polymers are linked through a polymerization-type reaction.

The solubilizing block (S) may comprise polymers comprising naturally occurring and/or non-natural monomers and combinations thereof.

In some embodiments, the solubilizing block (S) is selected from natural biopolymers. Natural biopolymers selected as solubilizing blocks (S) may include peptides (sometimes referred to as poly(amino acids)) comprising hydrophilic amino acids. Non-limiting examples of hydrophilic amino acids include serine, sulfo-serine, glutamic acid, aspartic acid, lysine, ornithine, arginine. Biopolymers can be selected from hydrophilic polysaccharides, which may include but are not limited to glycogen, cellulose, dextran, alginate and chitosan.

Monomers comprising the solubilizing block (S) can be selected from acrylates, (meth)acrylates, acrylamides, (meth)acrylamides, allyl ethers, vinyl acetates, vinyl amides, substituted styrenes, amino acids, acrylonitrile, heterocyclic monomers (e.g., ethylene oxide), saccharides, phosphoesters, phosphonamides, sulfonate esters, sulfonamides, or combinations thereof. Specific examples of (meth)acrylate and (meth)acrylamide monomers include N-2-hydroxypropyl(methacrylamide) (HPMA) and hydroxyethyl(methacrylate) (HEMA). Various monomers suitable for the solubilizing block (S) are described below.

In certain embodiments, the solubilizing block (S) comprises hydrophilic polymers selected from synthetic or natural poly(saccharides), such as glycogen, cellulose, dextran, alginate and chitosan. Hydrophilic polymers used as the solubilizing block (S) should have sufficient length to provide adequate surface coverage to stabilize particles formed by amphiphiles, e.g., amphiphiles of formula S-[B]-[U]-H. In preferred embodiments of solubilizing blocks comprising hydrophilic polymers, the hydrophilic polymer comprises 50 or monomer units, such as between 50 to 300, though, preferably between 50 and 100.

Solubilizing blocks (H) comprising linear polymers may comprise homopolymers comprising a single monomer composition or copolymers having two or more distinct compositions of monomers. In some embodiments, the homopolymer comprises neutral, hydrophilic monomers or charged monomers, e.g., positive, negative or zwitterion monomers. In other embodiments, the copolymer comprises neutral, hydrophilic monomers, and positive, negative or zwitterion monomers, or any combination thereof. Solubilizing blocks comprising linear polymers may comprise monomers linked to any solubilizing groups (SG) (or “moieties”), which generally refers to any hydrophilic groups, including neutral hydrophilic groups that do not carry a full integer value of charge; zwitterions, which are neutral but carry a whole number value of positive charge and a whole number value of negative charge; positively charged groups; and negatively charged groups; or a combination thereof

In some embodiments, the solubilizing block (S) comprises neutral hydrophilic monomers, which may be described generically as hydrophilic monomers. In some embodiments, the hydrophilic monomers are selected from (meth)acrylates or (meth)acrylamides (inclusive of acrylates, methacrylates, acrylamides and methacrylamides) of the chemical formula CH₂═CR¹¹—C(O)—R¹⁰ (“Formula II”), wherein the acryl side group R¹⁰ may be selected from one or more of —OR², —NHR¹² or —N(CH₃)R¹², where R¹¹ can be H or CH₃, and R¹² is independently selected from any hydrophilic substituent. Non-limiting examples of R¹² include but are not limited to H (except for OR¹³), CH₃, CH₂CH₃, CH₂CH₂OH, CH₂(CH₂)₂₀H, CH₂CH(OH)CH₃, CHCH₃CH₂OH or (CH₂CH₂O)_{y}H, where y is an integer number of repeating units, typically 1 to 6, such as 1, 2, 3, 4, 5 or 6.

A non-limiting example of a neutral hydrophilic monomer of Formula II wherein R¹”=NHR¹², R¹¹═CH₃, and R¹³═CH₂CH(OH)CH₃ is N-2-hydroxypropyl(methacrylamide) (HPMA):

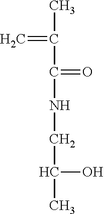

The above example, N-(2-hydroxpropyl(methacrylamide)) (HPMA), is an example of a neutral hydrophilic monomer of Formula II.

In some embodiments, the solubilizing block (S) comprises charged monomers that contain one or more functional groups (“charged functional group”) that either have a fixed charge or have net charge under certain physiological conditions. Non-limiting examples of charged monomers include any monomer that comprises amine, quaternary ammonium, sulfonic acid, sulfuric acid, sulfonium, phosphoric acid, phosphonic acid, phosphonium, carboxylic acid and/or boronic acid functional groups, as well as any combinations or salt forms thereof.

In some embodiments, charged monomers are selected from (meth)acrylates and (meth)acrylamides with chemical formula CH₂═CR¹⁴—C(O)—R¹³ (“Formula III”). The acryl side group R¹³ may be selected from one or more of the groups consisting of —OR¹⁵, —NHR¹⁵ or —N(CH₃)R¹⁵ where R¹⁴ can be H or CH₃ and R¹⁵ can be selected from, but is not limited to, H, linear alkyl structures such as (CH₂)_{y}NH₂, (CH₂)_{y}-imidazole, (CH₂)_{y}-pyridine amine, (CH₂)_{y}-(quinoline-amine), (CH₂)_{y}-pyridine amine, (CH₂)_{y}-naphthalene amine, (CH₂)_{y}CH(NH₂)COOH, (CH₂)_{y}COOH, (CH₂)_{y}CH(CH₃)COOH, (CH₂)_{y}C(CH₃)₂COOH, (CH₂)_{y}PO₃H₂, (CH₂)_{y}OPO₃H₂, (CH₂)_{y}SO₃H, (CH₂)_{y}OSO₃H, (CH₂)_{y}B(OH)₂, CH₂N(CH₃)₂, CH₂CH₂N(CH₃)₂, CH₂CH₂CH₂N(CH₃)₂, CH₂N(CH₂CH₃)₂, CH₂CH₂N(CH₂CH₃)₂, CH₂CH₂CH₂N(CH₂CH₃)₂, CH₂N(CH(CH₃)₂), CH₂CH₂N((CH(CH₃)₂), CH₂CH₂CH₂N(CH(CH₃)₂), CH[CH₂N(CH₃)₂]₂, CH(COOH)CHCH₂COOH, (CH₂)_{y}NH(CH₂)ⱼCOOH, (CH₂)_{y}N(CH₃)(CH₂)_{y}COOH, (CH₂)_{y}N⁺(CH₃)₂(CH₂)_{y}COOH, (CH₂)_{y}N⁺(CH₂—CH₃)₂(CH₂)_{y}COOH, [CH₂CH(CH₃)O]₅PO₃H₂, C(CH₃)₂CH₂SO₃H, and C₆H₄B(OH)₂ where y is an integer number of a repeating units, typically between 1 to 6, such as 1, 2, 3, 4, 5 or 6. In some embodiments of (meth)acrylates and (meth)acrylamides of Formula III, the acryl side group comprises tetraalkyl ammonium salts, nitrogen containing heterocycles, aminoaryl, or aminoheteroaryl, which may be linked to the monomer through any suitable means either directly or via a linker. Non-limiting examples of aryls, nitrogen containing heteroaryls and/or amino heteroaryls include pyrrolyl, imidazolyl, pyridinyl, pyrimidinyl, pyrazinyl, diazepinyl, indolyl, quinolinyl, amino quinolinyl, amino pyridinyl, purinyl, pteridinyl, anilinyl, amino naphthyl or the like. In certain preferred embodiments of (meth)acrylates and (meth)acrylamides of Formula III, the acryl side group comprises carboxylic acid(s), which may be linked to the monomer through any suitable means either directly or via a linker. A non-limiting example of a charged monomer of Formula III wherein R¹³═—OR¹⁵, R⁴═CH₃ and R¹⁵═H is:

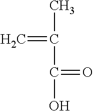

### Dendron-Based Solubilizing Blocks

Certain preferred embodiments of solubilizing blocks (S) comprise dendron amplifiers (“dendrons”), wherein the focal point of the solubilizing block (S) is linked either directly or indirectly via a spacer (B) and/or Linker U to a hydrophobic block (H), and the terminal groups (FGt) are either blind ended (unlinked) and function as solubilizing groups, or the terminal functional groups (FGt) are linked to solubilizing groups, wherein the solubilizing groups (SG) (or “moieties”) generally refer to any hydrophilic groups, including neutral hydrophilic groups that do not carry a full integer value of charge; zwitterions, which are neutral but carry a whole number value of positive charge and a whole number value of negative charge; positively charged groups; and negatively charged groups; or a combination thereof. In some embodiments, the solubilizing block (B) comprises dendron architecture and the terminal functional groups (FGt) are unlinked and therefore FGt are the solubilizing groups (SG). In other embodiments, the solubilizing block (B) comprises dendron architecture and the terminal functional groups (FGt) are linked either directly or via a linker to a solubilizing group (SG).

An unexpected finding reported herein is that the architecture and composition of amphiphiles of formula S-[B]-[U]-H had a marked impact on particle stability and drug loading into such particles. Accordingly, it was observed by the authors of the present disclosure that amphiphiles of formula S-[B]-[U]-H comprising solubilizing blocks with dendron architecture formed nanoparticles with improved hydrodynamic stability, higher drug loading and increased biological activity as compared with amphiphiles of formula S-[B]-[U]-H comprising solubilizing blocks (S) with linear architecture. Therefore, in preferred embodiments of amphiphiles, the amphiphile comprising a solubilizing block (S) further comprising a dendron amplifier, with a single (“core” or “focal point”) functional group linked either directly or indirectly via a spacer (B) and/or linker (U) to a hydrophobic block (H), additionally wherein the dendron has 2 or more solubilizing groups (SG), preferably, between 2 and 32 solubilizing groups, though more preferably between 4 and 8 solubilizing groups. Preferred compositions of dendron-based solubilizing blocks (S) are described throughout the specification.

The solubilizing groups (SG) comprising solubilizing blocks (S) with dendron architecture function to improve solubility and therefore stability of particles formed by amphiphiles but also impact blood protein interactions, cellular uptake and intracellular trafficking, which impact pharmacokinetics as well as safety and efficacy. Therefore, solubilizing groups (SG) should be carefully selected to meet the demands of the application.

It was identified that particular solubilizing group (SG) compositions that led to unexpected improvements in biological activity. Accordingly, particles comprising amphiphiles with solubilizing groups comprising dendrons with solubilizing groups (SG) selected from carboxylic acids with net negative charge (at pH 7.4) were found to be efficiently phagocytosed by monocyte populations. In contrast, particles comprising amphiphiles with solubilizing groups comprising linear polymers or dendrons with net neutral or near neutral charge were generally found to be poorly phagocytosed by immune cells, e.g., antigen presenting cells, and other cell populations, unless the linear polymers or dendrons comprise neutral sugar molecules that bind C-type lectin receptors that promote uptake by immune cell populations or other sugar molecules, such as glucose or galactose, which promote uptake via GLUT1 and asialoglycoprotein, respectively, by various cell populations. Furthermore, particles comprising amphiphiles with solubilizing groups comprising linear polymers or dendrons with net positive charge were found to be broadly taken up by various cell populations, particularly by antigen presenting cells. Thus, the solubilizing block (S) charge and composition can be tuned by varying the solubilizing groups (SG) to modulate biological activity. Preferred compositions of solubilizing groups are described below and throughout the specification.

### Linear Poly(Amino Acid)-Based Solubilizing Blocks

In some embodiments, the solubilizing block (S) is a linear poly(amino acid) comprising charged amino acids, hydrophilic amino acids or a combination thereof. Solubilizing blocks (S) comprising poly(amino acids) may be linked via the N- or C-termini or a side chain either directly or indirectly via a spacer (B) and/or linker (U). In some embodiments, a solubilizing block (S) comprising poly(amino acids) is linked to peptide antigen conjugates either directly or indirectly via an extension (E1 or E2) and/or Linker U. Solubilizing blocks comprising poly(amino acids) may comprise amino acids linked to any solubilizing groups (SG) (or “moieties”), which generally refers to any hydrophilic groups, including neutral hydrophilic groups that do not carry a full integer value of charge; zwitterions, which are neutral but carry a whole number value of positive charge and a whole number value of negative charge; positively charged groups; and negatively charged groups; or a combination thereof.

In certain embodiments, the solubilizing block (S) has a net negative charge and comprises 1 or more negatively charged amino acids. In certain embodiments, the solubilizing block (S) with a net negative charge comprises between 1 to 20 negatively charged amino acids, e.g., 1, 2, 3, 4, 5, 6, 7, 8, 9, 10, 11, 12, 13, 14, 15, 16, 17, 18, 19 or 20, though, more preferably between about 2 to 12 negatively charged amino acids.

In non-limiting examples, a poly(amino acid) comprising 12 aspartic acid monomers, e.g., Asp-Asp-Asp-Asp-Asp-Asp-Asp-Asp-Asp-Asp-Asp-Asp (SEQ ID NO:32), is used to prepare a solubilizing block (S) with a net negative charge of −12; a poly(amino acid) comprising 11 aspartic acid monomers, e.g., Asp-Asp-Asp-Asp-Asp-Asp-Asp-Asp-Asp-Asp-Asp (SEQ ID NO:33), is used to prepare a solubilizing block (S) with a net negative charge of −11; a poly(amino acid) comprising 10 aspartic acid monomers, e.g., Asp-Asp-Asp-Asp-Asp-Asp-Asp-Asp-Asp-Asp (SEQ ID NO:34), is used to prepare a solubilizing block (S) with a net negative charge of −10; a poly(amino acid) comprising 9 aspartic acid monomers, e.g., Asp-Asp-Asp-Asp-Asp-Asp-Asp-Asp-Asp (SEQ ID NO:35), is used to prepare a solubilizing block (S) with a net negative charge of −9; a poly(amino acid)) comprising 8 aspartic acid monomers, e.g., Asp-Asp-Asp-Asp-Asp-Asp-Asp-Asp (SEQ ID NO:36), is used to prepare a solubilizing block (S) with a net negative charge of −8; a poly(amino acid) comprising 7 aspartic acid monomers, e.g., Asp-Asp-Asp-Asp-Asp-Asp-Asp (SEQ ID NO:37), is used to prepare a solubilizing block (S) with a net negative charge of −7; a poly(amino acid) comprising 6 aspartic acid monomers, e.g., Asp-Asp-Asp-Asp-Asp-Asp (SEQ ID NO:38), is used to prepare a solubilizing block (S) with a net negative charge of −6; a poly(amino acid) comprising 5 aspartic acid monomers, e.g., Asp-Asp-Asp-Asp-Asp (SEQ ID NO:39), is used to prepare a solubilizing block (S) with a net negative charge of −5; a poly(amino acid) comprising 4 aspartic acid monomers, e.g., Asp-Asp-Asp-Asp (SEQ ID NO:40), is used to prepare a solubilizing block (S) with a net negative charge of −4; a poly(amino acid) comprising 3 aspartic acid monomers, e.g., Asp-Asp-Asp, is used to prepare a solubilizing block (S) with a net negative charge of −3; a poly(amino acid) comprising 2 aspartic acid monomers, e.g., Asp-Asp, is used to prepare a solubilizing block (S) with a net negative charge of −2. In the above examples, aspartic acid (Asp) may be replaced with any suitable negatively charged amino acid, including but not limited to glutamic acid, sulfo-serine, or phospho-serine, wherein the negatively charged amino acids may be the same or different.

In certain embodiments, the solubilizing block (S) has a net positive charge and comprises 1 or more positively charged amino acids. In certain embodiments, the solubilizing block (S) with a net positive charge comprises between 1 to 20 positively charged amino acids, e.g., 1, 2, 3, 4, 5, 6, 7, 8, 9, 10, 11, 12, 13, 14, 15, 16, 17, 18, 19 or 20, though, more preferably between about 2 to 12 positively charged amino acids. In preferred embodiments of vaccines, wherein the at least one peptide antigen conjugate has net positive charge, the peptide antigen conjugate comprises a solubilizing block (S) that further comprises between 1 to 20 positively charged amino acids.

In non-limiting examples, a poly(amino acid) comprising 12 lysine monomers, e.g., Lys-Lys-Lys-Lys-Lys-Lys-Lys-Lys-Lys-Lys-Lys-Lys (SEQ ID NO:41), is used to prepare a solubilizing block (S) with a net positive charge of +12; a poly(amino acid) comprising 11 lysine monomers, e.g., Lys-Lys-Lys-Lys-Lys-Lys-Lys-Lys-Lys-Lys-Lys (SEQ ID NO:42), is used to prepare a solubilizing block (S) with a net positive charge of +11; a poly(amino acid) comprising 10 lysine monomers, e.g., Lys-Lys-Lys-Lys-Lys-Lys-Lys-Lys-Lys-Lys (SEQ ID NO:43), is used to prepare a solubilizing block (S) with a net positive charge of +10; a poly(amino acid) comprising 9 lysine monomers, e.g., Lys-Lys-Lys-Lys-Lys-Lys-Lys-Lys-Lys (SEQ ID NO:44), is used to prepare a solubilizing block (S) with a net positive charge of +9; a poly(amino acid) comprising 8 lysine monomers, e.g., Lys-Lys-Lys-Lys-Lys-Lys-Lys-Lys (SEQ ID NO:45), is used to prepare a solubilizing block (S) with a net positive charge of +8; a poly(amino acid) comprising 7 lysine monomers, e.g., Lys-Lys-Lys-Lys-Lys-Lys-Lys (SEQ ID NO:46), is used to prepare a solubilizing block (S) with a net positive charge of +7; a poly(amino acid) comprising 6 lysine monomers, e.g., Lys-Lys-Lys-Lys-Lys-Lys (SEQ ID NO:47), is used to prepare a solubilizing block (S) with a net positive charge of +6; a poly(amino acid) comprising 5 lysine monomers, e.g., Lys-Lys-Lys-Lys-Lys (SEQ ID NO:48), is used to prepare a solubilizing block (S) with a net positive charge of +5; a poly(amino acid) comprising 4 lysine monomers, e.g., Lys-Lys-Lys-Lys (SEQ ID NO:49), is used to prepare a solubilizing block (S) with a net positive charge of +4; a poly(amino acid) comprising 3 lysine monomers, e.g., Lys-Lys-Lys, is used to prepare a solubilizing block (S) with a net positive charge of +3; a poly(amino acid) comprising 2 lysine monomers, e.g., Lys-Lys, is used to prepare a solubilizing block (S) with a net positive charge of +2. In the above examples, Lysine (Lys) may be replaced with any suitable positively charged amino acid, including but not limited to trimethyl-lysine, ornithine or arginine, wherein the positively charged amino acids may be the same or different. In preferred embodiments of vaccines, wherein the at least one peptide antigen conjugate has net positive charge, the peptide antigen conjugate comprises a solubilizing block (S) that further comprises between 1 to 20 positively charged amino acids that comprise primary amines, including but not limited to lysine and ornithine.

### Zwitterion Peptides

In additional embodiments, the solubilizing block (H) comprises both negatively and positively charged amino acids, or amino acids with both positively and negatively charged functional groups. Dipeptides comprising amino acids of opposite charge, e.g., Lys-Asp, are referred to as zwitterion dipeptides because they are predicted to have a net neutral, 0, charge at pH 7.4. One or more zwitterion dipeptides can be included in the solubilizing block (S) as a means to i) improve water solubility and ii) provide a prevailing charge (e.g., net negative or net positive) over certain pH ranges. For instance, a zwitterion di-peptide can be used to increase the hydrophilic character of a peptide sequence without increasing or decreasing the charge of a peptide sequence at pH 7.4. However, the zwitterion can be used to impart a net charge at a particular pH. For instance, excluding the contribution of the N-terminal amine and the C-terminal carboxylic acid in this example, the zwitterion di-peptide, Lys-Asp, has a net charge of 0 at pH 7.4, but a net charge of +1 at pH<4 and a net charge of −1 at pH>10. One or more zwitterion di-peptides can be added to the sequence of poly(amino acid)-based solubilizing blocks; for example, one di-peptide, Lys-Asp; two di-peptides Lys-Asp-Lys-Asp (SEQ ID NO:50); three di-peptides, Lys-Asp-Lys-Asp-Lys-Asp (SEQ ID NO:51) and so forth. In the above examples, Lysine (Lys) may be replaced with any suitable positively charged amino acid, including but not limited to trimethyl-lysine, ornithine or arginine, and aspartic acid (Asp) may be replaced with any suitable negatively charged amino acid, including but not limited to glutamic acid, sulfo-serine, or phospho-serine, wherein the positively or negatively charged amino acids may be the same or different.

The solubilizing block (S) comprising poly(amino acids) may additionally comprise small non-charged, hydrophilic amino acids, or hydrophilic linkers, e.g., ethylene oxide that function to i) improve water solubility and ii) increase the distance between charged functional groups to prevent incomplete ionization. For instance, ionization of one functional group on a polymer may impact the pKa of neighboring functional groups through local effects. For example, protonation of an amine in close proximity to a second amine may cause a reduction in the pKa of the conjugate acid of the second amine. To reduce the impact of local effects on the ionization potential of neighboring functional groups, a linker molecule may be used to increase the distance between charged functional groups. The linker molecule may comprise between 1 to 5 small, non-charged hydrophilic amino acids, e.g., 1, 2, 3, 4, and 5 amino acids. Alternatively, the linker may comprise an ethylene oxide (i.e., PEG) linker between 1 to 4, or more, monomer units, e.g., 1, 2, 3, or 4 ethylene oxide monomers in length. In certain embodiments of solubilizing blocks comprising poly(amino acids), 1 to 2 non-bulky, non-charged hydrophilic amino acids are placed between neighboring charged amino acids, wherein the amino acids are linked through amide bonds. In certain embodiments, a serine is placed between all or some of the charged amino acids comprising the poly(amino acid)-based solubilizing block (S).

### Solubilizing Groups (SG)

Solubilizing groups (SG) (or “moieties”) are defined broadly as any hydrophilic groups, including neutral hydrophilic groups that do not carry a full integer value of charge; zwitterions, which are neutral but carry a whole number value of positive charge and a whole number value of negative charge; positively charged groups; and negatively charged groups; or a combination thereof. In certain preferred embodiments, the solubilizing block (B) comprises solubilizing groups (SG) selected from sugar molecules comprising one or more sugar monomers, e.g., monosaccharides, disaccharides, trisaccharides, oligosaccharides and the like. Non-limiting examples of solubilizing groups selected from sugar molecules include but are not limited to glucose, glucosamine, N-acetyl glucosamine, galactose, galactosamine, N-acetyl galactosamine, mannose and sialyl lewis^{X} (sLeX), which may be linked to solubilizing blocks through any suitable linker at any suitable attachment point, e.g.:

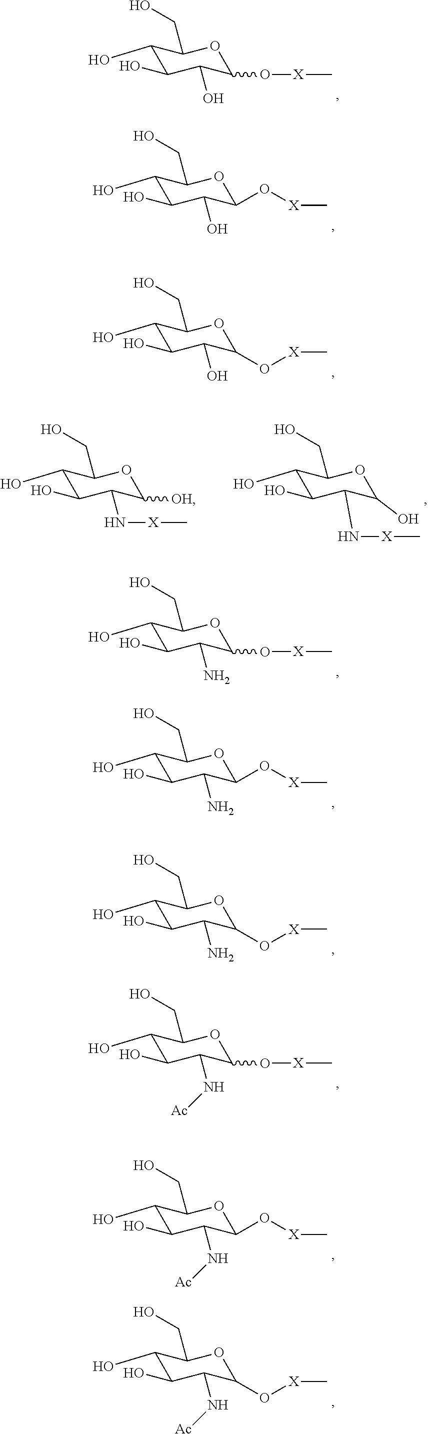

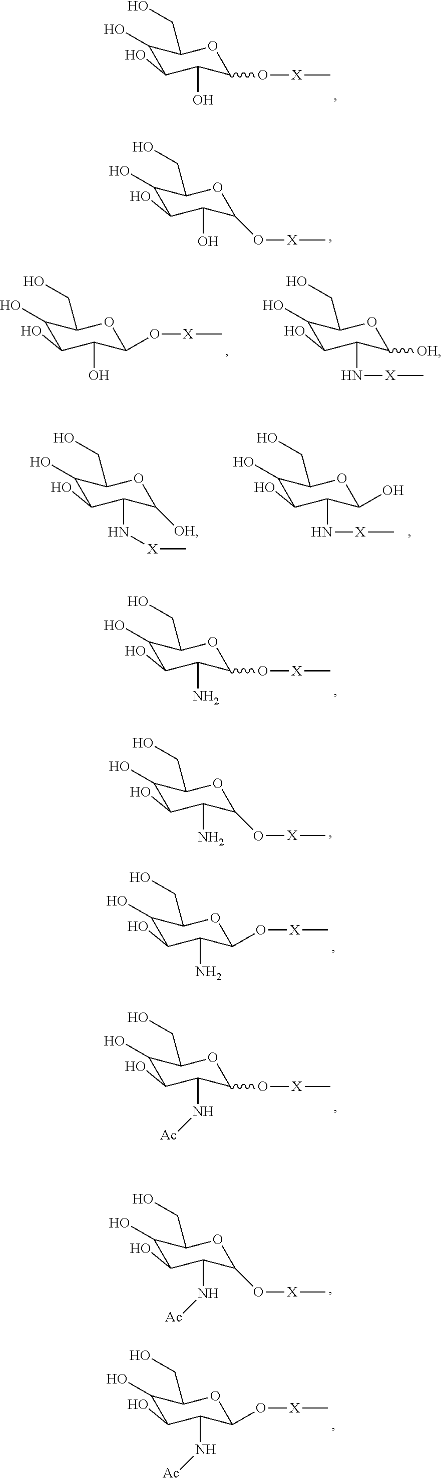

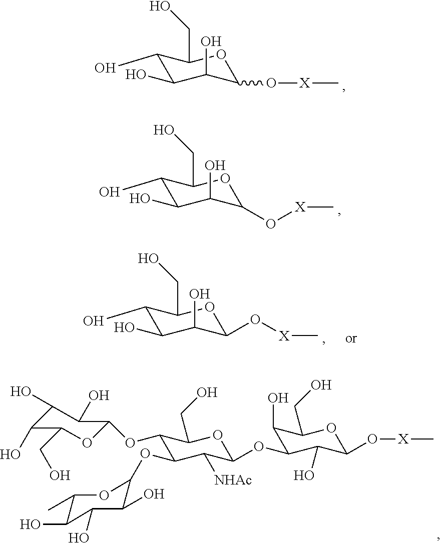

wherein X is any suitable linker molecule, which may be present or absent, and when present is typically selected from lower alkyl or PEG groups.

In some embodiments, the solubilizing block (S) comprises solubilizing groups (SG) that have net positive or net negative charge in aqueous buffers at a pH of about 7.4. The charge of the solubilizing groups (SG) may be dependent or independent of the pH of the solution in which the solubilizing block (S) is dispersed, such is the case, for example, for tertiary amines and quaternary ammonium compounds that are pH dependent and pH independent, respectively. Non-limiting examples of solubilizing groups that have net positive or net negative charge at certain pH in aqueous solutions or have pH independent charge are provided here for clarity:

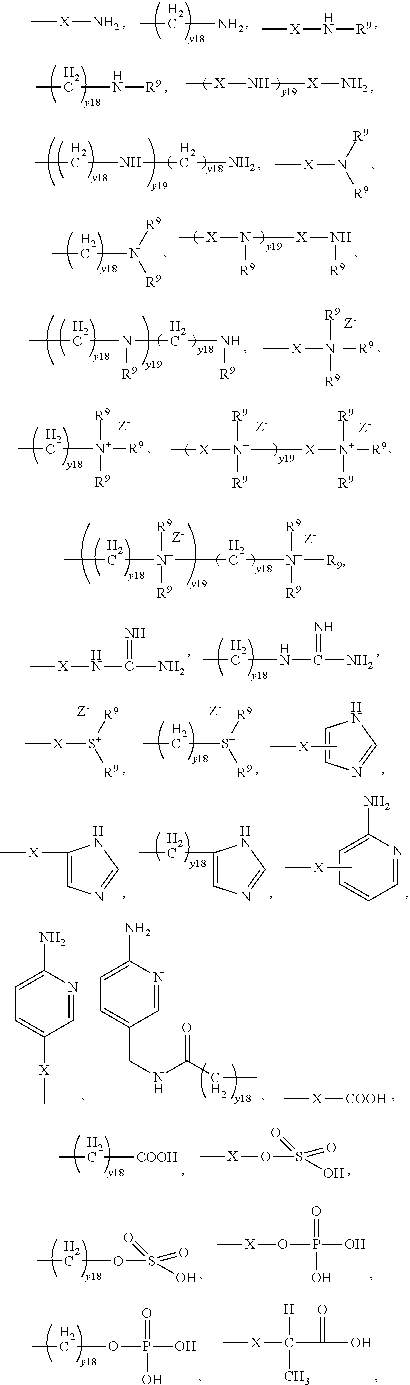

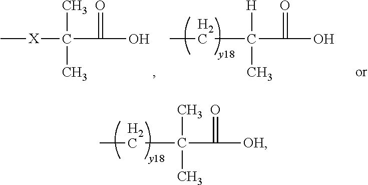

wherein X is any suitable linker molecule, which may be present or absent, and when present is typically selected from lower alkyl or PEG, y18 and y19 are each independently any integer, typically selected from between 1 to 6, R⁹ is selected from lower alkyl or branched alkyl groups, such as CH₃, CH₂CH₃, CH₂CH₂CH₃, CH(CH₃)₂, H₂CH(CH₃)₂ or the like, and Z⁻ is any suitable counter anion, which is typically selected from conjugate bases of weak acids or halide ions, such as Cl⁻, I⁻, or Br⁻.

In certain preferred embodiments, the solubilizing block (S) comprises solubilizing groups (SG) selected from zwitterions that have 0 net charge, or net 0 charge in aqueous conditions at certain pH. In some embodiments, the solubilizing block (S) comprises solubilizing groups (SG) selected from zwitterions that have 0 net charge at pH 7.4, but have net positive charge at reduced pH, e.g., tumor pH between about 5.5 to 7.0. Non-limiting examples of solubilizing groups comprising zwitterions are provided here for clarity:

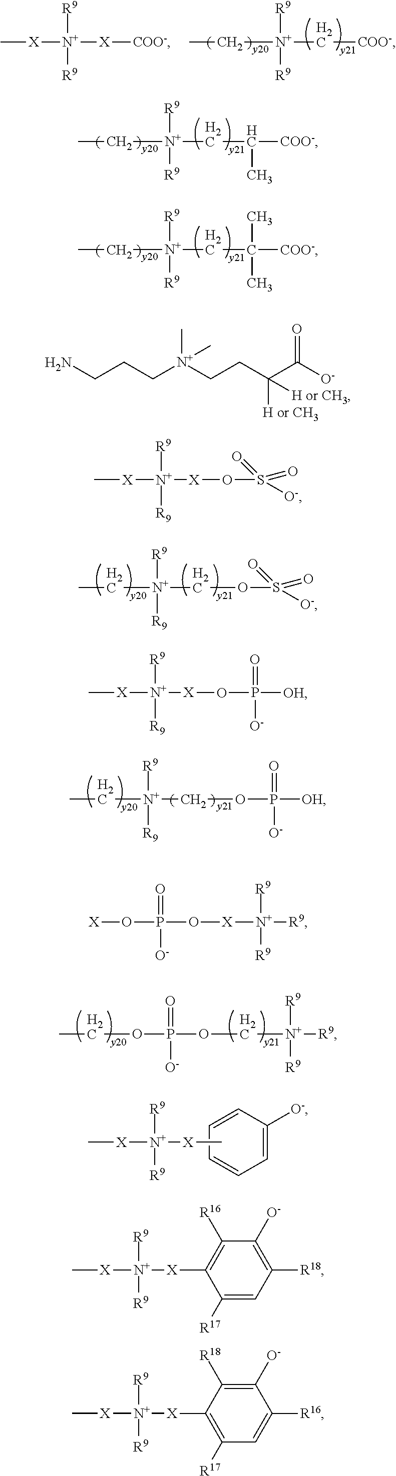

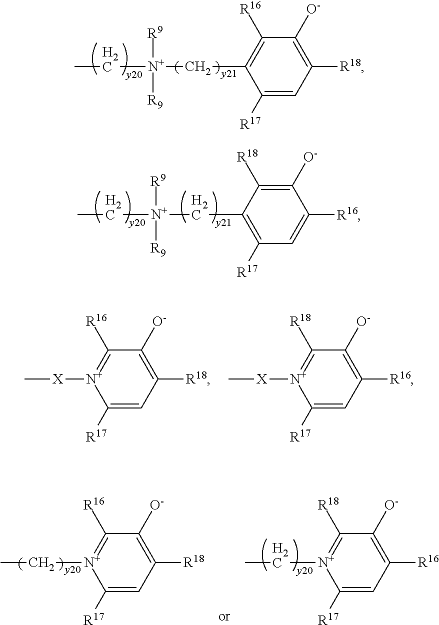

wherein X is any suitable linker, which may be present or absent, and when present is typically selected from lower alkyl or PEG groups, y20 and y21 are each independently any integer, typically selected from between 1 to 6, R⁹ is selected from lower alkyl or branched alkyl groups, such as CH₃, CH₂CH₃, CH₂CH₂CH₃, CH(CH₃)₂, H₂CH(CH₃)₂ or the like, R¹⁶, R¹⁷ and R¹⁸ are each independently selected from —H, CH₃, F and —NO₂.

In some embodiments, the solubilizing group (SG) may further comprise a targeting moiety and/or drug molecule. As a non-limiting example, certain sugar molecules may improve solubility and therefore function as a solubilizing group; additionally, the sugar molecule may bind to cell surface receptors and/or exert a physiological effect and therefore also function as a targeting moiety and/or drug molecule (D). Accordingly, solubilizing groups (SG) comprising mannose bind to mannose receptors and therefore target cells and tissues expressing such receptors; additionally, binding to the mannose receptor can promote phagocytosis and may therefore exert a physiological effect. Additional non-limiting examples of solubilizing groups (SG) that may perform two or more functions include targeting molecules comprising hydrophilic peptides, glycopeptides, antibodies, fragments of antibodies, nanobodies, nucleic acid aptamers and related molecules that are both hydrophilic and bind to specific cells or tissues.

### Linkage of Solubilizing Group (SG) to the Solubilizing Block

Solubilizing groups (SG) may be linked to the solubilizing block (S) through any suitable means, including any suitable linker molecule. In certain preferred embodiments of dendron-based solubilizing blocks (S), the terminal functional group is a carboxylic acid, and the solubilizing group is linked via an ester or, more preferably, an amide bond. In certain other preferred embodiments of dendron-based solubilizing blocks (S), the terminal functional group is an amine, and the solubilizing group is linked to the terminal functional group via an amide or carbamate bond.

In preferred embodiments, solubilizing groups (SG) are linked to the solubilizing block (S) through a covalent bond via a suitable linker X, which is typically selected from lower alkyl or PEG groups. Particular suitable linkers X that are preferred for joining SG to S are referred to as X5. In non-limiting examples, solubilizing blocks (S) selected from either polymers comprising monomers comprising amines or dendrons comprising terminal functional groups (FGt) comprising amines, e.g., —NH₂, are covalently linked to solubilizing groups (SG) via a suitable linker, X5, through reaction with activated carboxylic acids (LG-C(O)—R¹⁹) to yield —NH—C(O)—R¹⁹; activated mixed carbonates (LG-C(O)—O—R¹⁹) or chloroformates (C₁—C(O)—O—R¹⁹) to yield NH—C(O)—O—R¹⁹; aldehydes or ketones (CR²²(O)—R¹⁹) to yield Schiff base of formula CR²²(—NH)—R¹⁹; alkenes (C(R²²)(R²³)═C(R²⁴)(R¹⁹) to yield Michael-addition products (e.g., NH-C(R²²)(R²³)—CH(R²⁴)(R¹⁹) or —N(C(R²²)(R²³)—CH(R²⁴)(R¹⁹))₂); or, alkyl or aryl halide (LG-R¹⁹, wherein LG=Cl, Br or I), to yield —NH—R¹⁹, —N(—R¹⁹)₂ and/or —N+(—R¹⁹)₃. In additional non-limiting examples, solubilizing blocks (S) selected from either polymers comprising monomers comprising carboxylic acids or dendrons comprising terminal functional groups (FGt) comprising carboxylic acids, e.g., —COOH (or —C(O)-LG), are covalently linked to solubilizing groups (SG) via a suitable linker, X5, through reaction with an amine (NH₂—R¹⁹) to yield —C(O)—NH—R¹⁹ or methylamine (R¹⁹—N(CH₃)(H) or R¹⁹—NHMe) to yield —C(O)—N(CH₃)(R¹⁹).

In the above non-limiting examples, LG is any suitable leaving group, and R¹⁹ may be selected from but is not limited to —(CH₂)ₜ-SG, —(CH₂CH₂O)ₜ—CH₂CH₂—SG, —(CH₂)ₜ—C(O)—NH—(CH₂)ᵤ-SG, —(CH₂CH₂O)ₜCH₂CH₂C(O)—NH—(CH₂)ᵤ-SG, —(CH₂)ₜ—NH—C(O)—NH—(CH₂)ᵤ-SG and (CH₂CH₂O)ₜCH₂CH₂NH—C(O)—(CH₂)ᵤ-SG where t and u are each independently an integer typically selected from between 1 to 6, such as 1, 2, 3, 4, 5 or 6. Preferred X5 for linking S to SG (i.e., S-X5-SG) are typically selected from —NH—(CH₂)ₜ—, —NH—(CH₂CH₂O)ₜ—CH₂CH₂—, —NH—(CH₂)ₜ—C(O)—NH—(CH₂)ᵤ—, —NH—(CH₂CH₂O)ₜCH₂CH₂C(O)—NH—(CH₂)ᵤ—, NH—(CH₂)ₜ—NH—C(O)—NH—(CH₂)ᵤ—, —NH(CH₂CH₂O)ₜCH₂CH₂NH—C(O)—(CH₂)ᵤ—, —C(O)—(CH₂)ₜ—, —C(O)—(CH₂CH₂O)ₜ—CH₂CH₂—, —C(O)—(CH₂)ₜ—C(O)—NH—(CH₂)ᵤ—, C(O)—(CH₂CH₂O)ₜCH₂CH₂C(O)—NH—(CH₂)ᵤ—, C(O)—(CH₂)ₜ—NH—C(O)—NH—(CH₂)ᵤ— or —C(O)—(CH₂CH₂O)ₜCH₂CH₂NH—C(O)—(CH₂)ᵤ—, where t and u are each independently an integer typically selected from between 1 to 6, such as 1, 2, 3, 4, 5 or 6.

A non-limiting example of an amphiphile comprising a solubilizing block (S) with dendron architecture, wherein the dendron is second generation and comprises monomeric units selected from FG1-CH(R¹)₂, wherein FG1 (and the focal point) is NH₂, R¹ is (OCH₂CH₂)_{y}-FG2, y is 1 and FG2 (and FGt) is COOH, wherein the terminal functional group (FGt) carboxylic acids are linked to NH₂—R¹⁹ to yield —C(O)—NH₂—R¹⁹ wherein R¹⁹ is —(CH₂CH₂O)ₜ—CH₂CH₂—SG, t=1 and the solubilizing group is selected from a glucose is provided below for clarity:

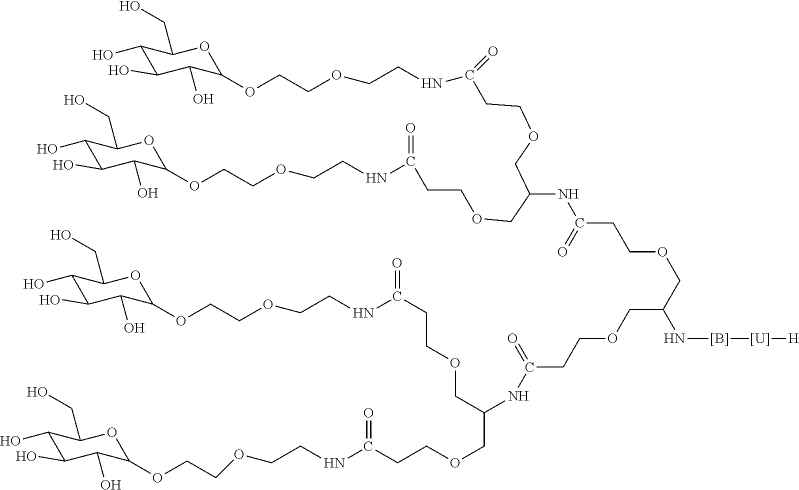

Wherein the solubilizing block (S) is linked either directly or indirectly via a spacer (B) and/or Linker U to the hydrophobic block (H), which may further comprise a drug molecule (e.g., H-D). In the above example, X5 is —NH—R¹⁹ and R¹⁹ is —(CH₂CH₂O)ₜ—CH₂CH₂—SG, which may be written as —NH—(CH₂CH₂O)ₜ—CH₂CH₂—(SG not shown), wherein t=1 and SG is a glucose.

Additional examples of hydrophobic blocks (H) with dendron architecture that have particular utility for certain applications and/or lead to unexpected improvements in manufacturing and/or biological activity are provided throughout the specification.

### Impact of the Number of Charged Functional Groups

In some embodiments, the solubilizing block (S) has a net negative charge and comprises one or more functional groups that carry a negative charge at pH 7.4. Suitable solubilizing blocks (S) that carry a net negative charge include molecules bearing functional groups (e.g., functional groups with a pKa of about 7.4 or less) that occur as the conjugate base of an acid at physiologic pH, at a pH of about 7.4 or less. These include but are not limited to molecules bearing carboxylates, sulfates, phosphates, phosphoramidates, and phosphonates. The solubilizing block (S) bearing a carboxylate may be selected from but is not limited to carboxylic acids selected from glutamic acid, aspartic acid, pyruvic acid, lactic acid, glycolic acid, glucuronic acid, citrate, isocitrate, alpha-keto-glutarate, succinate, fumarate, malate, oxaloacetate, butyrate, methylbutyrate, dimethylbutyrate and derivatives thereof. In certain embodiments, the solubilizing block (S) comprises a molecule with between 1 to 20 negatively charged functional groups, such as 1, 2, 3, 4, 5, 6, 7, 8, 9, 10, 11, 12, 13, 14, 15, 16, 17, 18, 19 or 20 negatively charged functional groups, though, typically no more than 8 negatively charged functional groups, preferably between 4 and 8 negatively charged functional groups.

In some embodiments, the solubilizing block (S) has a net positive charge and comprises positively charged functional groups. Suitable solubilizing blocks (S) that carry a net positive charge include molecules that occur as the conjugate acid of weak bases at pH 7.4, wherein the pKa of the conjugate acid of the base is greater than 7.4. These include but are not limited to molecules bearing primary, secondary and tertiary amines, as well as quaternary ammonium, guanidinium, phosphonium and sulfonium functional groups. Suitable molecules bearing ammonium functional groups include, for example, imidazolium, and tetra-alkyl ammonium compounds. In some embodiments, the solubilizing block comprises quaternary ammonium or sulfonium compounds that carry a permanent positive charge that is independent of pH.

In some embodiments, the solubilizing group (S) comprises between 1-20 positively charged functional groups, such as 1, 2, 3, 4, 5, 6, 7, 8, 9, 10, 11, 12, 13, 14, 15, 16, 17, 18, 19 or 20 positively charged functional groups. For amphiphiles, the solubilizing block (S) typically has no more than 8 charged functional groups, preferably between 4 and 8 positively charged functional groups.

For peptide antigen conjugates, the number of charged functional groups of the solubilizing block (S) is typically selected to ensure net charge of the peptide antigen conjugate at physiologic pH 7.4 is greater than or equal to +2 or greater than or equal to +3, and the solubilizing block is typically selected from poly(amino acids) comprising lysine or ornithine. For compositions of vaccines comprising at least one peptide antigen conjugate comprising a solubilizing block (S) but lacking an amphiphilic carrier molecule (i.e., the amphiphile, e.g., S-[B]-[U]-H is absent) the number of charged functional groups present on the solubilizing block (S) of the peptide antigen conjugate is typically selected to ensure net charge of the peptide antigen conjugate at physiologic pH 7.4 is greater than or equal to +5 or greater than equal to +6, preferably between +6 and +12 and more preferably between +8 and +10, and the solubilizing block is typically selected from poly(amino acids) comprising lysine or ornithine. For compositions of vaccines comprising at least one peptide antigen conjugate comprising a solubilizing block, wherein the vaccine further comprises an amphiphilic carrier molecule (“amphiphile,” e.g., of formula S-[B]-[U]-H), the number of charged functional groups present on the solubilizing block (S) of the peptide antigen conjugate is typically selected to ensure net charge of the peptide antigen conjugate at physiologic pH 7.4 is greater than or equal to +2 or greater than equal to +3, though, typically no more than +10, and the solubilizing block is typically selected from poly(amino acids) comprising lysine or ornithine. For compositions of vaccines meant for intravenous administration, wherein the at least one peptide antigen conjugate comprises a solubalizing block (S) and wherein the vaccine further comprises an amphiphilic carrier molecule (“amphiphile,” e.g., of formula S-[B]-[U]-H), the number of charged functional groups present on the solubilizing block (S) of the peptide antigen conjugate is typically selected to ensure net charge of the peptide antigen conjugate at physiologic pH 7.4 is greater than or equal to +2 or greater than equal to +3, but typically no more than +6, more preferably between +3 and +5, and the solubilizing block is typically selected from poly(amino acids) comprising lysine or ornithine. The process for designing and manufacturing peptide antigen conjugates to achieve a specific net charge has been described by Lynn and colleagues (see: Lynn et al., Nature Biotechnology. 2020) and in patent application WO2018187515, which are incorporated by reference herein in their entirety.

### Counter-Ion Selection

An additional consideration regarding charged molecules (C) is the counterion selected. Non-limiting examples of charged molecules (C) bearing functional groups with positive charge include but are not limited to halides, including chloride, bromide and iodide anions, and conjugate bases of acids, including, phosphate, sulfates, sulfites and carboxylate anions including formate, succinate, acetate and trifluoroacetate. Suitable counterions for charged molecules (C) bearing functional groups with negative charge include but are not limited to hydrogen and alkali and alkaline earth metals, including, for example, sodium, potassium, magnesium and calcium, or conjugate acids of weak bases, such as ammonium compounds. Suitable amines used to form the ammonium salt include but are not limited to ammonium, primary amines, such as tris(hydroxymethyl)aminomethane (“TRIS”), secondary amines based on di-alkyl amines, such as dimethyl amine and diethyl amine, tertiary amines based on tri-alkyl amines, such as trimethylamine, di-isopropyl ethylamine (DIPEA) and triethylamine (TEA), as well as quaternary ammonium compounds. Unexpectedly, tris(hydroxymethyl)aminomethane (or Tris) as the ammonium salt of acids as the counterion of amphiphilic block copolymers with negative charge has improved solubility in both water-miscible organic solvents, such as DMSO, DMF, acetone and ethanol, and aqueous solutions. For these reasons, the protonated form of tris(hydroxymethyl)aminomethane is a preferred counter-ion to use in the preparation of salts of conjugate bases of acids present on the amphiphilic block copolymers of the present disclosure.

### Antigens

The antigen of immunogenic compositions, e.g., vaccines, may be any antigen that is useful for inducing an immune response in a subject and is often selected from peptide antigens (A) but may also be selected from small molecules (sometimes referred to as haptens). The peptide antigen (A) may be used to induce either a proinflammatory or tolerogenic immune response depending on the nature of the immune response required for the application. In some embodiments, the peptide antigen (A) is a tumor-associated antigen, such as a self-antigen, neoantigen or tumor-associated viral antigen (e.g., HPV E6/E7). In other embodiments, the peptide antigen (A) is an infectious disease antigen, such as a peptide derived from a protein isolated from a virus, bacteria, fungi or protozoan microbial pathogen. In some embodiments, the peptide antigen (A) is a peptide derived from an allergen or an autoantigen, which is known or suspected to cause allergies or autoimmunity. In some embodiments, the peptide antigen (A) is a self-antigen selected from proteins involved in cardiovascular diseases. In still other embodiments, the peptide antigen (A) is an analog of a toxins and/or recreational drugs.

The peptide antigen (A) comprises a sequence of amino acids or a peptide mimetic that can induce an immune response, such as a T cell or B cell response in a subject. In some embodiments, the peptide antigen (A) comprises an amino acid or amino acids with a post-translational modification (e.g., glycosylation, oxidation, phosphorylation, citrullination and/or homocitrullination), non-natural amino acids or peptide-mimetics. The peptide antigen may be any sequence of natural, non-natural or post-translationally modified amino acids, peptide-mimetics, or any combination thereof, that have an antigen or predicted antigen, i.e., an antigen with a T cell and/or B cell epitope. Peptide antigens (A) also include post-translationally modified peptide antigens (A), including glycopeptides.

The inventors of the present disclosure found unexpectedly that replacing certain amino acids, e.g., Cysteine and Methionine, found in naturally occurring peptide antigen sequences with amino acids that are not naturally found in those sequences, e.g., alpha aminobutyric acid (aBut) and norleucine (nLeu), respectively, led to unexpected improvements in vaccine manufacturing and in vivo immunogenicity. Therefore, in preferred embodiments of peptide antigens (A), naturally occurring cysteine amino acids are replaced with alpha aminobutyric acid and methionine amino acids are replaced with norleucine.

Immunogenic compositions, including compositions of vaccines, e.g., vaccine for inducing tolerance, may comprise one or more different peptide antigen conjugates each having a different peptide antigen (A) composition. In some embodiments, the immunogenic compositions comprise particles with up to 50 different peptide antigen conjugates each having a unique peptide antigen (A) composition. In some embodiments, the immunogenic compositions comprise mosaic particles that comprise two or more different peptide antigen conjugates, e.g., up to about 100 different peptide antigen conjugates, typically no more than about 40 peptide antigen conjugates, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 peptide antigen conjugates. In other embodiments, the immunogenic compositions comprise mosaic particles that comprise 5 different peptide antigen conjugates. In still other embodiments, the immunogenic compositions comprise a single particle composition comprising of a single (1) peptide antigen conjugate composition.

The length of the peptide antigen (A) depends on the specific application and is typically between about 5 to about 100 amino acids. In preferred embodiments, the peptide antigen (A) is between about 7 to 35 amino acids, e.g., 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids. In still other cases, the peptide antigen is a full-length polypeptide, such as a protein antigen that may be recombinantly expressed. Peptide antigens (A) based on full-length tumor-associated antigens, infectious disease antigens, allergens, autoantigens or self-antigens that cause cardiovascular or neurodegenerative disease may be delivered as the full-length sequence, or as an overlapping peptide pool wherein each peptide antigen (A) of the overlapping peptide pool is no more than 100 amino acids in length, preferably no more than 35 amino acids in length.

In some embodiments, the peptide antigen (A) is 7 to 35 amino acids, typically about 25. Thus, for a tumor-associated antigen, infectious disease antigen, allergen or auto-antigen greater than 25 amino acids in length, e.g., a 100 amino acid antigen, the antigen may be divided into 7 to 35 amino acid, e.g., 25 amino acid, peptide antigens (A) wherein each peptide antigen (A) contains a unique composition of amino acids; or, the peptide antigens (A) can be overlapping peptide pools wherein an antigen is divided into a set number of 7 to 35 amino acid, e.g., 25 amino acid, peptide antigens (A) that have overlapping sequences. For example, an overlapping peptide pool comprising a 100 amino acid antigen may be divided into eight 25 amino acid peptide antigens (A) that are each offset by 12 amino acids (i.e., each subsequent 25 amino acid peptide comprising a 100 amino acid peptide sequence starts at the 13^{th} amino acid position from the prior peptide). Those skilled in the art understand that many permutations exist for generating a peptide pool from an antigen.

In some embodiments, the peptide antigen (A) is a minimal CD8 or CD4 T cell epitope that comprises the portions of a tumor-associated antigen, infectious disease antigen, allergen or autoantigen that are predicted in silico (or measured empirically) to bind MHC-I or MHC-II molecules. Algorithms for predicting MHC-I or MHC-II binding are widely available (see Lundegaard et al., Nucleic Acids Res., 36:W509-W512, 2008 and http://www.cbs.dtu.dk/services/NetMHC/). In some embodiments of a personalized therapy for a particular subject, the peptide antigen (A) comprising a peptide antigen conjugate may comprise a minimal CD8 T cell epitope from a tumor-associated antigen, infectious disease antigen, allergen or autoantigen that is typically a 7-13 amino acid peptide that is predicted to have <1,000 nM binding affinity for a particular MHC-I allele that is expressed by that subject. In some embodiments of a personalized therapy for a particular subject, the peptide antigen (A) may comprise a minimal CD4 T cell epitope from a tumor-associated antigen, infectious disease antigen, allergen or autoantigen that is an 8-20 amino acid peptide, or more preferably a 10-16 amino acid peptide, that is predicted to have <1,000 nM binding affinity for a particular MHC-II allele that is expressed by that subject. In certain preferred embodiments, when a minimal CD8 or CD4 T cell epitope cannot be identified for a tumor-associated antigen, infectious disease antigen, allergen or autoantigen, or when the tumor-associated antigen, infectious disease antigen, allergen or autoantigen contains multiple CD8 and CD4 T cell epitopes, the peptide antigen (A) may be between 16-35 amino acids, e.g., up to 35 amino acids such that it may contain all possible CD8 or CD4 T cell epitopes.

In some embodiments, the peptide antigen (A) is derived from tumor antigens. Tumor antigens include self-antigens that are present on healthy cells but are preferentially expressed by tumor cells, or neoantigens, which are aberrant proteins that are specific to tumor cells and are unique to individual patients. Tumor antigens may also include viral antigens.

Preferred self-antigens include antigens that are preferentially expressed by tumor cells, such as CLPP, Cyclin-A1, MAGE-A1, MAGE-C1, MAGE-C2, SSX2, XAgE1b/GAGED2a, Melan-A/MART-1, TRP-1, Tyrosinase, CD45, glypican-3, IGF2B3, Kallikrein 4, KIF20A, Lengsin, Meloe, MUC5AC, survivin, prostatic acid phosphatase, NY-ESO-1 and MAGE-A3.

Neoantigens arise from the inherent genetic instability of cancers, which can lead to mutations in DNA, RNA splice variants and changes in post-translational modification, all potentially leading to de novo protein products that are referred to collectively as neoantigens or sometimes predicted neoantigens. DNA mutations include changes to the DNA including nonsynonymous missense mutations, nonsense mutations, insertions, deletions, chromosomal inversions and chromosomal translocations, all potentially resulting in novel gene products and therefore neoantigens. RNA splice site changes can result in novel protein products and missense mutations can introduce amino acids permissive to post-translational modifications (e.g., phosphorylation) that may be antigenic. The instability of tumor cells can furthermore result in epigenetic changes and the activation of certain transcription factors that may result in selective expression of certain antigens by tumor cells that are not expressed by healthy, non-cancerous cells.

Peptide antigen conjugates used in personalized cancer vaccines should include peptide antigens (A) that comprise the portions of tumor-associated antigens that are unique to tumor cells. Peptide antigens (A) comprising neoantigens arising from a missense mutation should encompass the amino acid change encoded by 1 or more nucleotide polymorphisms. Peptide antigens (A) comprising neoantigens that arise from frameshift mutations, splice site variants, insertions, inversions and deletions should encompass the novel peptide sequences and junctions of novel peptide sequences. Peptide antigens (A) comprising neoantigens with novel post-translational modifications should encompass the amino acids bearing the post-translational modification(s), such as a phosphate or glycan. In preferred embodiments, the peptide antigen (A) comprises the up to 25 amino acids on either side flanking the amino acid change or novel junction that arises due to a mutation. In certain embodiments, the peptide antigen (A) is a neoantigen sequence that comprises the 12 amino acids on either side flanking the amino acid change that arises from a single nucleotide polymorphism, for example, a 25 amino acid peptide, wherein the 13^{th} amino acid is the amino acid residue resulting from the single nucleotide polymorphism. In some embodiments, the peptide antigen (A) is a neoantigen sequence that comprises the 12 amino acids on either side flanking an amino acid with a novel post-translational modification, for example, a 25 amino acid peptide, wherein the 13^{th} amino acid is the amino acid residue resulting from the novel post-translational modification site. In other embodiments, the peptide antigen (A) is a neoantigen sequence that comprises 0-12 amino acids on either side flanking a novel junction created by an insertion, deletion or inversion. In some cases, the peptide antigen (A) comprising neoantigens resulting from novel sequences can encompass the entire novel sequence, including 0-25 amino acids on either side of novel junctions that may also arise.

Tumor-associated antigens suitable as peptide antigens (A) for immunogenic compositions of the present disclosure can be identified through various techniques that are familiar to one skilled in the art. Tumor-associated antigens can be identified by assessing protein expression of tumor cells as compared with healthy cells, i.e., non-cancerous cells from a subject. Suitable methods for assessing protein expression include but are not limited to immunohistochemistry, immunofluorescence, western blot, chromatography (i.e., size-exclusion chromatography), ELISA, flow cytometry and mass spectrometry. Proteins preferentially expressed by tumor cells but not healthy cells or by a limited number of healthy cells (e.g., CD20) are suitable tumor-associated antigens. DNA and RNA sequencing of patient tumor biopsies followed by bioinformatics to identify mutations in protein-coding DNA that are expressed as RNA and produce peptides predicted to bind to MHC-I or MHC-II alleles on patient antigen presenting cells (APCs), may also be used to identify tumor-associated antigens that are suitable as peptide antigens (A) for immunogenic compositions of the present disclosure.

In preferred embodiments, tumor-associated antigens suitable as peptide antigens (A) for immunogenic compositions are identified using mass spectrometry. Suitable peptide antigens (A) are peptides identified by mass spectrometry following elution from the MHC molecules from patient tumor biopsies but not from healthy tissues from the same subject (i.e., the peptide antigens are only present on tumor cells but not healthy cells from the same subject). Mass spectrometry may be used alone or in combination with other techniques to identify tumor-associated antigens. Those skilled in the art recognize that there are many methods for identifying tumor-associated antigens, such as neoantigens (see Yadav et al., Nature, 515:572-576, 2014) that are suitable as peptide antigens (A) for the practice of the disclosed invention.

In preferred embodiments, the tumor-associated antigens used as peptide antigens (A) are clonal or nearly clonal within the population of neoplastic cells, which may be considered heterogeneous in other respects.

Tumor-associated antigens selected for use as peptide antigens (A) in personalized cancer vaccination schemes may be selected based on mass spectrometry confirmation of peptide-MHC binding and/or in silico predicted MHC binding affinity and RNA expression levels within tumors. These data provide information on whether or not a tumor-associated antigen is expressed and presented by tumor cells and would therefore be a suitable target for T cells. Such criteria may be used to select the peptide antigens (A) used in a personalized cancer vaccine.

For patients with highly mutated tumors that have more than 50 tumor-associated neoantigens, a down-selection process may be used to select peptide antigens (A) for use in personalized cancer vaccines comprising peptide antigen conjugates. In some embodiments, a down-selection process is used to select peptide antigens (A) comprising epitopes predicted to have the highest MHC binding affinity and RNA expression levels within tumor cells. Additional criteria may be applied for the selection of tumor-associated self-antigens or neoantigens. For example, predicted immunogenicity or predicted capacity of the peptide antigen (A) to lead to T cells that react with other self-antigens, which may lead to autoimmunity, are additional criteria considered. For instance, peptide antigens (A) that comprise tumor-associated antigens and have high predicted immunogenicity but also low potential to lead to autoimmunity are criteria used to select potential peptide antigens (A) for use in personalized cancer vaccines. In some embodiments, neoantigens that that would be expected to result in T cell or antibody responses that react with self-antigens found on healthy cells are not selected for use as peptide antigens (A). For patients with less than, for example, 20-50 predicted neoantigens, a down selection process may not be critical and so all 20-50 predicted neoantigens might be used as peptides antigens (A) in a personalized cancer vaccine.

Cancer vaccines may include peptide antigens (A) that comprise tumor-associated antigens that are patient-specific and/or tumor-associated antigens that are shared between patients. For example, the tumor-associated antigen can be a conserved self-antigen, such as NY-ESO-1 (testicular cancer) or gp100 (melanoma), or the antigen may be a cryptic epitope, such as Na17 (melanoma) that is not typically expressed by healthy cells but is conserved between certain cancer patients. Immunogenic compositions of the present disclosure may include peptide antigens (A) that arise from so-called hot-spot mutations that are frequent mutations in certain genes or gene regions that occur more frequently than would be predicted by chance. Non-limiting examples of hot spot mutations include the V600E mutation in BRAF protein, which is common to melanoma, papillary thyroid and colorectal carcinomas, or KRAS G12 mutations, which are among the most common mutations, such as KRAS G12C. A number of suitable self-antigens as well as neoantigens that arise from hotspot mutations are known and are incorporated herein by reference: see Chang et al., Nature Biotechnology, 34:155-163, 2016; Vigneron, N., et al, Cancer Immunology, 13:15-20, 2013.

In some embodiments, the peptide antigen (A) can be from a hematological tumor. Non-limiting examples of hematological tumors include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

In some embodiments, the peptide antigen (A) can be from a solid tumor. Non-limiting examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma). In several examples, a tumor is melanoma, lung cancer, lymphoma breast cancer or colon cancer.

In some embodiments, the peptide antigen (A) is a tumor-associated antigen from a breast cancer, such as a ductal carcinoma or a lobular carcinoma. In some embodiments, the peptide antigen (A) is a tumor-associated antigen from a prostate cancer. In some embodiments, peptide antigen (A) is a tumor-associated antigen from a skin cancer, such as a basal cell carcinoma, a squamous cell carcinoma, a Kaposi's sarcoma, or a melanoma. In some embodiments, the peptide antigen (A) is a tumor-associated antigen from a lung cancer, such as an adenocarcinoma, a bronchioalveolar carcinoma, a large cell carcinoma, or a small cell carcinoma. In some embodiments, the peptide antigen (A) is a tumor-associated antigen from a brain cancer, such as a glioblastoma or a meningioma. In some embodiments, the peptide antigen (A) is a tumor-associated antigen from a colon cancer. In some embodiments, the peptide antigen (A) is a tumor-associated antigen from a liver cancer, such as a hepatocellular carcinoma. In some embodiments, the peptide antigen (A) is a tumor-associated antigen from a pancreatic cancer. In some embodiments, peptide antigen (A) is a tumor-associated antigen from a kidney cancer, such as a renal cell carcinoma. In some embodiments, the peptide antigen (A) is a tumor-associated antigen from a testicular cancer.

In some embodiments, the peptide antigen (A) is a tumor-associated antigen derived from premalignant conditions, such as variants of carcinoma in situ, or vulvar intraepithelial neoplasia, cervical intraepithelial neoplasia, or vaginal intraepithelial neoplasia.

In some embodiments, the peptide antigen (A) is an antigen from an infectious agent, such as a virus, a bacterium, or a fungus. In additional embodiments, the peptide antigen (A) is a peptide or glycopeptide derived from an infectious agent; for example, the HIV Envelope fusion peptide or a V3 or V1/V2 glycopeptide from HIV. In some embodiments, the antigen is a peptide antigen derived from a flavivirus, such as dengue, West Nile virus, Zika virus, hepatitis C or others; a coronavirus, such as MERS, SARS or SARS-COV-2 viruses; a paramyxovirus, such as a mumps, measles, respiratory syncytial virus (RSV), human parainfluenza viruses, as well as zoonotic viruses, such a Newcastle disease virus; a filovirus, such as Ebola or Marburg viruses; human papillomaviruses; hepadnaviruses, such as hepatitis B; orthomyxovirus, such as influenza; lentiviruses, such as HIV; and other viral derived proteins or glycoproteins.

In certain preferred embodiments of vaccines against HPV, the peptide antigens are selected from MHQKRTAMFQDPQERPRKLPQLCTELQTT (SEQ ID NO:136), PRKLPQLCTELQTTIHDIILECVYCKQQL (SEQ ID NO:137), HDIILECVYCKQQLLRREVYDFAFRDLCI (SEQ ID NO: 138), RREVYDFAFRDLCIVYRDGNPYAVCDKCL (SEQ ID NO: 139), YRDGNPYAVCDKCLKFYSKISEYRHYCYS (SEQ ID NO:140), FYSKISEYRHYCYSLYGTTLEQQYNKPLC (SEQ ID NO:141), YGTTLEQQYNKPLCDLLIRCINCQKPLCP (SEQ ID NO:142), LLIRCINCQKPLCPEEKQRHLDKKQRFHN (SEQ ID NO:143), EKQRHLDKKQRFHNIRGRWTGRCMSCCR (SEQ ID NO:144), IRGRWTGRCMSCCRSSRTRRETQL (SEQ ID NO: 145), MHGDTPTLHEYMLDLQPETTDLYCYEQ (SEQ ID NO: 146), DLQPETTDLYCYEQLNDSSEEEDEI (SEQ ID NO: 147), YEQLNDSSEEEDEIDGPAGQAEPDR (SEQ ID NO:148), DEIDGPAGQAEPDRAHYNIVTFCCKCD (SEQ ID NO:149), RAHYNIVTFCCKCDSTLRLCVQSTHVDIRTLE (SEQ ID NO:150), LCVQSTHVDIRTLEDLLMGTLGIVCPICSQKP (SEQ ID NO:151) and QLYQTCKAAGTCPSDVIPKI (SEQ ID NO:152). In preferred embodiments of vaccines against HPV, one or more cysteine and/or methionine residues of naturally occurring peptide antigens are replaced with alpha-aminobutyric acid (“B”) and/or norleucine (“n”), respectively. Non-limiting examples include nHQKRTAnFQDPQERPRKLPQLBTELQTT (SEQ ID NO:153) nHQKRTAnFQDPQERPRKLPQLCTELQTT (SEQ ID NO: 154), MHQKRTAMFQDPQERPRKLPQLBTELQTT (SEQ ID NO:155), PRKLPQLBTELQTTIHDIILEBVYBKQQL (SEQ ID NO:156), HDIILEBVYBKQQLLRREVYDFAFRDLBI (SEQ ID NO: 157), RREVYDFAFRDLBIVYRDGNPYAVBDKBL (SEQ ID NO: 158), YRDGNPYAVBDKBLKFYSKISEYRHYBYS (SEQ ID NO:159), FYSKISEYRHYBYSLYGTTLEQQYNKPLB (SEQ ID NO: 160), YGTTLEQQYNKPLDLLIRBINBQKPLBP (SEQ ID NO:161), LLIRBINBQKPLBPEEKQRHLDKKQRFHN (SEQ ID NO:162), EKQRHLDKKQRFHNIRGRWTGRCnSCCR (SEQ ID NO:163), EKQRHLDKKQRFHNIRGRWTGRBnSBBR (SEQ ID NO:164), EKQRHLDKKQRFHNIRGRWTGRBMSBBR (SEQ ID NO:165), IRGRWTGRCnSCCRSSRTRRETQL (SEQ ID NO: 166), IRGRWTGRBnSBBRSSRTRRETQL (SEQ ID NO:167), IRGRWTGRBMSBBRSSRTRRETQL (SEQ ID NO:168), nHGDTPTLHEYnLDLQPETTDLYCYEQ (SEQ ID NO:169), nHGDTPTLHEYnLDLQPETTDLYBYEQ (SEQ ID NO:170), nHGDTPTLHEYnLDLQPETTDLYMYEQ (SEQ ID NO:171), DLQPETTDLYBYEQLNDSSEEEDEI (SEQ ID NO: 172), YEQLNDSSEEEDEIDGPAGQAEPDR (SEQ ID NO:173), DEIDGPAGQAEPDRAHYNIVTFBBKBD (SEQ ID NO:174), RAHYNIVTFBBKBDSTLRLBVQSTHVDIRTLE (SEQ ID NO:175), LCVQSTHVDIRTLEDLLnGTLGIVCPICSQKP (SEQ ID NO:176), LBVQSTHVDIRTLEDLLnGTLGIVBPIBSQKP (SEQ ID NO:177), LBVQSTHVDIRTLEDLLMGTLGIVBPIBSQKP (SEQ ID NO:178) and QLYQTBKAAGTBPSDVIPKI (SEQ ID NO: 179) or any fragments thereof having at least 6 amino acids in length, preferably at least 9 amino acids, or derivatives thereof.

In certain other preferred embodiments of vaccines against HPV, including HPV+cancers, the peptide antigens are selected from ALQAIELQLTLETIYNSQYSNEKWTLQDV (SEQ ID NO:180), NSQYSNEKWTLQDVSLEVYLTAPTGCIKK (SEQ ID NO:181), SVTVVEGQVDYYGLYYVHEGIRTYFVQFK (SEQ ID NO:182), LKGDANTLKCLRYRFKKHCTLYTAVSSTWHWT (SEQ ID NO:183), KHKSAIVTLTYDSEWQRDQFLSQVKIPKT (SEQ ID NO:184), MHQKRTAMFQDPQERPRKLPQLCTELQTT (SEQ ID NO:185), PRKLPQLCTELQTTIHDIILECVYCKQQL (SEQ ID NO:186), HDIILECVYCKQQLLRREVYDFAFRDLCI (SEQ ID NO: 187), RREVYDFAFRDLCIVYRDGNPYAVCDKCL (SEQ ID NO: 188), YRDGNPYAVCDKCLKFYSKISEYRHYCYS (SEQ ID NO:189), FYSKISEYRHYCYSLYGTTLEQQYNKPLC (SEQ ID NO: 190), YGTTLEQQYNKPLCDLLIRCINCQKPLCP (SEQ ID NO:191), CPEEKQRHLDKKQRFHNIRGRWTGRCMSCCR (SEQ ID NO:192), MHGDTPTLHEYMLDLQPETTDLYCYEQ (SEQ ID NO: 193), AGQAEPDRAHYNIVTFCCKCDSTLRLCVQ (SEQ ID NO: 194) and LCVQSTHVDIRTLEDLLMGTLGIVCPICSQKP (SEQ ID NO:195), wherein in preferred embodiments one or more cysteine and/or methionine residues are replaced with alpha-aminobutyric acid and/or norleucine, respectively, for example, ALQAIELQLTLETIYNSQYSNEKWTLQDV (SEQ ID NO:180), NSQYSNEKWTLQDVSLEVYLTAPTGBIKK (SEQ ID NO: 196), SVTVVEGQVDYYGLYYVHEGIRTYFVQFK (SEQ ID NO:182), LKGDANTLKBLRYRFKKHBTLYTAVSSTWHWT (SEQ ID NO:197), KHKSAIVTLTYDSEWQRDQFLSQVKIPKT (SEQ ID NO:184), nHQKRTAnFQDPQERPRKLPQLBTELQTT (SEQ ID NO: 198), PRKLPQLBTELQTTIHDIILEBVYBKQQL (SEQ ID NO:199), HDIILEBVYBKQQLLRREVYDFAFRDLBI (SEQ ID NO:200), RREVYDFAFRDLBIVYRDGNPYAVBDKBL (SEQ ID NO:201), YRDGNPYAVBDKBLKFYSKISEYRHYBYS (SEQ ID NO:202), FYSKISEYRHYBYSLYGTTLEQQYNKPLB (SEQ ID NO:203), YGTTLEQQYNKPLBDLLIRBINBQKPLBP (SEQ ID NO:204), BPEEKQRHLDKKQRFHNIRGRWTGRBnSBBR (SEQ ID NO:205), nHGDTPTLHEYnLDLQPETTDLYBYEQ (SEQ ID NO:206), AGQAEPDRAHYNIVTFBBKBDSTLRLBVQ (SEQ ID NO:207) and LBVQSTHVDIRTLEDLLnGTLGIVBPIBSQKP (SEQ ID NO:208).

In some embodiments of cancer vaccines for prostate cancer, the cancer vaccine comprises peptide antigens selected from fragments of prostate specific antigen (PSA), APLILSRIVGGWECEKHSQPWQVLVASRGRAVCGGVLVHPQWVLTAAHCIRNKSVILLGRHS LFHPEDTGQVFQVSHSFPHPLYDMSLLKNRFLRPGDDSSHDLMLLRLSEPAELTDAVKVMDL PTQEPALGTTCYASGWGSIEPEEFLTPKKLQCVDLHVISNDVCAQVHPQKVTKFMLCAGRWT GGKSTCSGDSGGPLVCNGVLQGITSWGSEPCALPERPSLYTKVVHYRKWIKDTIVANP (SEQ ID NO:209). In certain preferred embodiments, the peptide antigens (A) selected from fragments of PSA are typically selected from 7 to 55 amino acid stretches of PSA that may optionally overlap. Non-limiting examples include but are not limited to:

(SEQ ID NO: 210)
CGGVLVHPQWVLTAAHCIRNKSVILLGRHSLFHPE,
(SEQ ID NO: 211)
SLFHPEDTGQVFQVSHSFPHPLYDMSLLKNRFLRP,
(SEQ ID NO: 212)
PCALPERPSLYTKVVHYRKWIKDTIVANP.

In some embodiments of cancer vaccines for prostate cancer, the cancer vaccine comprises peptide antigens selected from fragments of prostatic acid phosphatase (PAP), FFWLDRSVLAKELKFVTLVFRHGDRSPIDTFPTDPIKESSWPQGFGQLTQLGMEQHYELGEYI RKRYRKFLNESYKHEQVYIRSTDVDRTLMSAMTNLAALFPPEGVSIWNPILLWQPIPVHTVPL SEDQLLYLPFRNCPRFQELESETLKSEEFQKRLHPYKDFIATLGKLSGLHGQDLFGIWSKVYDP LYCESVHNFTLPSWATEDTMTKLRELSELSLLSLYGIHKQKEKSRLQGGVLVNEILNHMKRA TQIPSYKKLIMYSAHDTTVSGLQMALDVYNGLLPPYASCHLTELYFEKGEYFVEMYYRNETQ HEPYPLMLPGCSPSCPLERFAELVGPVIPQDWSTECMTTNSHQGTEDSTD (SEQ ID NO:213).

In certain preferred embodiments, the peptide antigens (A) selected from fragments of PAP are typically selected from 7 to 55 amino acid stretches of PAP that may optionally overlap. Non-limiting examples include but are not limited to: RTLMSAMTNLAALFPPEGVSIWNPILLWQPIPVHT (SEQ ID NO:214), PILLWQPIPVHTVPLSEDQLLYLPFRNCPRFQELE (SEQ ID NO:215), ATEDTMTKLRELSELSLLSLYGIHKQKEKSRLQGG (SEQ ID NO:216), LQGGVLVNEILNHMKRATQIPSYKKLIMYSAHDTT (SEQ ID NO:217), MALDVYNGLLPPYASCHLTELYFEKGEYFVEMYYR (SEQ ID NO:218), YFEKGEYFVEMYYRNETQHEPYPLMLPGCSPSCPL (SEQ ID NO:219).

In some embodiments of cancer vaccines for prostate cancer, the cancer vaccine comprises peptide antigens selected from fragments of STEAP1, MESRKDITNQEELWKMKPRRNLEEDDYLHKDTGETSMLKRPVLLHLHQTAHADEFDCPSEL QHTQELFPQWHLPIKIAAIIASLTFLYTLLREVIHPLATSHQQYFYKIPILVINKVLPMVSITLLA LVYLPGVIAAIVQLHNGTKYKKFPHWLDKWMLTRKQFGLLSFFFAVLHAIYSLSYPMRRSYR YKLLNWAYQQVQQNKEDAWIEHDVWRMEIYVSLGIVGLAILALLAVTSIPSVSDSLTWREFH YIQSKLGIVSLLLGTIHALIFAWNKWIDIKQFVWYTPPTFMIAVFLPIVLIFKSILFLPCLRKKILK IRHGWEDVTKINKTEICSQL (SEQ ID NO:220). In certain preferred embodiments, the peptide antigens (A) selected from fragments of STEAP1 are typically selected from 7 to 55 amino acid stretches of STEAP1 that may optionally overlap. Non-limiting examples include but are not limited to: LFPQWHLPIKIAAIIASLTFLYTLLREVIHPLATS (SEQ ID NO:221), YTLLREVIHPLATSHQQYFYKIPILVINKVLPMVS (SEQ ID NO:222), RKQFGLLSFFFAVLHAIYSLSYPMRRSYRYKLLNWAYQ (SEQ ID NO:223), EDAWIEHDVWRMEIYVSLGIVGLAILALLAVTSIP (SEQ ID NO:224), LAVTSIPSVSDSLTWREFHYIQSKLGIVSLLLGTI (SEQ ID NO:225), DIKQFVWYTPPTFMIAVFLPIVLIFKSILFLPCLR(SEQ ID NO:226).

In some embodiments of cancer vaccines for prostate cancer, the cancer vaccine comprises peptide antigens selected from fragments of 5T4, SSPTSSASSFSSSAPFLASAVSAQPPLPDQCPALCECSEAARTVKCVNRNLTEVPTDLPAYVRN LFLTGNQLAVLPAGAFARRPPLAELAALNLSGSRLDEVRAGAFEHLPSLRQLDLSHNPLADLS PFAFSGSNASVSAPSPLVELILNHIVPPEDERQNRSFEGMVVAALLAGRALQGLRRLELASNHF LYLPRDVLAQLPSLRHLDLSNNSLVSLTYVSFRNLTHLESLHLEDNALKVLHNGTLAELQGLP HIRVFLDNNPWVCDCHMADMVTWLKETEVVQGKDRLTCAYPEKMRNRVLLELNSADLDC DPILPPSLQTSYVFLGIVLALIGAIFLLVLYLNRKGIKKWMHNIRDACRDHMEGYHYRYEINA DPRLTNLSSNSDV (SEQ ID NO:227). In certain preferred embodiments, the peptide antigens (A) selected from fragments of 5T4 are typically selected from 7 to 55 amino acid stretches of 5T4 that may optionally overlap. Non-limiting examples include but are not limited to:

(SEQ ID NO: 228)
SPTSSASSESSSAPFLASAVSAQPPLPDQCPALCE,
(SEQ ID NO: 229)
RNLTEVPTDLPAYVRNLFLTGNQLAVLPAGAFARR,
(SEQ ID NO: 230)
ALQGLRRLELASNHFLYLPRDVLAQLPSLRHLDLS,
(SEQ ID NO: 231)
LSNNSLVSLTYVSFRNLTHLESLHLEDNALKVLHN,
(SEQ ID NO: 232)
DCDPILPPSLQTSYVFLGIVLALIGAIFLLVLYLN.

In certain preferred embodiments of vaccines against influenza, the peptide antigens are selected from minimal immunogens, including but not limited to RNNILRTQESE (SEQ ID NO:233) and LNDKHSNGTIKDRSPYR (SEQ ID NO:234), SWRNNILRTQES (SEQ ID NO:235), DNWHGSNRP (SEQ ID NO:236), DNPRPNDKTGS (SEQ ID NO:237) and DPNGWTGTDNNFSI (SEQ ID NO:238) or any fragments thereof having at least 6 amino acids in length, preferably at least 9 amino acids, or derivatives thereof.

In certain preferred embodiments of vaccines against hepatitis B, the peptide antigens are selected from minimal immunogens, including but not limited to QLDPAFRAG (SEQ ID NO:239), RGLYFPAGL (SEQ ID NO:240) and STGPCRTCMTK (SEQ ID NO:241) or any fragments thereof having at least 6 amino acids in length, preferably at least 9 amino acids, or derivatives thereof.

In certain preferred embodiments of vaccines against HIV, the peptide antigens are selected from minimal immunogens, including but not limited to AVGIGAVFL (SEQ ID NO:242), EINCTRPNNNTRPGEIIGDIRQAHCNISRA (SEQ ID NO:243) or YNKRKRIHIGPGRAFYTTKNIIG (SEQ ID NO:244).

In certain preferred embodiments of vaccines against malaria, the peptide antigens are selected from minimal immunogens including but not limited to PADGNPDPNANPNVD (SEQ ID NO:245), NPDPNANPNVDPNAN (SEQ ID NO:246), NANPNVDPNANPNVD (SEQ ID NO:247), NANPNANPNANPNAN (SEQ ID NO:248), DPNANPNVDPNA (SEQ ID NO:249), KQPADGNPDPNANPNV (SEQ ID NO:250), EDNEKLRKPKHKKLKQPADGNPDPNANPNVDPNAN (SEQ ID NO:251), KLRKPKHKKLKQPADGNPDP (SEQ ID NO:252) or any fragments thereof having at least 6 amino acids in length, preferably at least 9 amino acids, or derivatives thereof.

In preferred embodiments of vaccines against SARS the peptide antigens are selected from TESNKKFLPFQQFGRDIA (SEQ ID NO:253), SQILPDPSKPSKRSFIEDLLFNKVTLADAGF (SEQ ID NO:254), SQILPDPSKPSKRSFIEDLLFNKVT (SEQ ID NO:255), PSKPSKRSFIEDLLFNKVTLADAGF (SEQ ID NO:256), DYSVLYNSASFSTFKCYGVSPTKLNDLCFTN (SEQ ID NO:257), LYNSASFSTFKCYGVSPTKL (SEQ ID NO:258), SNNLDSKVGGNYNYLYRLFRKSNLK (SEQ ID NO:259), YRLFRKSNLKPFERDISTEIYQAGS (SEQ ID NO:260), ISTEIYQAGSTPCNGVEGFNCYFPL (SEQ ID NO:261), VEGFNCYFPLQSYGFQPTNGVGYQ (SEQ ID NO:262) and SNNLDSKVGGNYNYLYRLFRGSGIYQAGSTPCNGVEGFNCYFPLQSYGFQPTNGVGYQ (SEQ ID NO:263) or any fragments thereof having at least 6 amino acids in length, preferably at least 9 amino acids, or derivatives thereof.

In some embodiments, the peptide antigen (A) represents an autoantigen. The autoantigen may be identified and selected on the basis of screening a subject's own T cells for autoreactivity against self-antigens presented in the context of a patient's own MHC-I and/or MHC-II molecules. Alternatively, the peptide antigens may be selected using in silico methods to predict potential autoantigens that (i) have a predicted high affinity for binding a subjects' own MHC-I and/or MHC-II molecules and (ii) are expressed and/or known to be associated with pathology accounting for a subject's autoimmune syndrome. In other embodiments, the peptide antigen represents a CD4 epitope derived from an allergen and is selected on the basis of the peptide antigen having a high binding affinity for a patient's own MHC-II molecules.

In some embodiments, the autoantigen is specific for the tissue that is being damaged by the autoimmune response. In some embodiments, the autoantigen is widely expressed. In some embodiments, the autoantigen is induced by inflammation, such as a heat shock protein. In all cases, the peptides may be selected from protein sequences comprising one or more isoforms as a result of splice variants or post-translational modifications or proteolytic processing. In some embodiments the antigen is an alloantigen selected from donor tissue and used to prevent or treat transplant rejection. Vaccine compositions for inducing tolerance described herein for autoantigens can also be used for alloantigens for preventing or treating transplant rejection. Compositions for inducing tolerance generally apply to any class of antigen useful for treating inflammatory diseases, which includes autoantigens, allergens and alloantigens.

In some embodiments wherein the composition is used to treat multiple sclerosis and related neuro-inflammatory diseases, the antigens are selected from peptide sequences from the myelin sheath proteins, including myelin basic protein (MBP), myelin oligodendrocyte protein (MBP), and/or myelin proteolipid protein (PLP). In some embodiments wherein the composition is used to treat type 1 diabetes, the antigens are selected from peptides that are expressed in pancreatic islet cells, including insulin, glutamic acid decarboxylase, chromogranin A, and/or neuropilin. In some embodiments wherein the composition is used to treat neuromyelitis optica, the antigen is aquaporin 4. In some embodiments wherein the composition is used to treat celiac disease, the antigen is gluten proteins and/or transglutaminase. In some embodiments wherein the composition is used to treat pemphigus vulgaris, the antigen is epidermal cadherin. In some embodiments wherein the composition is used to treat myasthenia gravis, the antigen is acetylcholine receptor. In some embodiments wherein the composition is used to treat allergy, the antigen is the allergen.

In some embodiments wherein the composition is used to mitigate the effects of anti-drug antibodies against therapeutically useful recombinant proteins or vectors, the antigen is the recombinant protein or a fragment thereof. Non-limiting examples include Factor VIII, gene delivery vectors such as adeno-associated viruses, vaccine viral vectors such as adenoviruses, monoclonal antibodies, or fusion proteins.

In some embodiments of tolerance vaccines used to treat multiple sclerosis, the antigen comprises one or more peptide fragments having at least six amino acids in length, more preferably at least 9 or more amino acids in length, derived from any isoform of myelin oligodendrocyte glycoprotein, such as

(SEQ ID NO: 264)
MASLSRPSLPSCLCSFLLLLLLQVSSSYAGQFRVIGPRHPIRALVGDEV
ELPCRISPGKNATGMEVGWYRPPFSRVVHLYRNGKDQDGDQAPEYRGRT
ELLKDAIGEGKVTLRIRNVRFSDEGGFTCFFRDHSYQEEAAMELKVEDP
FYWVSPGVLVLLAVLPVLLLQITVGLIFLCLQYRLRGKLRAEIENLHRT
FDPHFLRVPCWKITLFVIVPVLGPLVALIICYNWLHRRLAGQFLEELRN
PF.

In some embodiments of tolerance vaccines used to treat multiple sclerosis, the antigen comprises one or more peptide fragments having at least six amino acids in length, more preferably at least 9 or more amino acids in length, derived from any isoform of myelin basic proteins, such as

(SEQ ID NO: 265)
MGNHAGKRELNAEKASTNSETNRGESEKKRNLGELSRTTSEDNEVFGEA
DANQNNGTSSQDTAVTDSKRTADPKNAWQDAHPADPGSRPHLIRLFSRD
APGREDNTFKDRPSESDELQTIQEDSAATSESLDVMASQKRPSQRHGSK
YLATASTMDHARHGFLPRHRDTGILDSIGRFFGGDRGAPKRGSGK.

In some embodiments of tolerance vaccines used to treat multiple sclerosis, the antigen comprises one or more peptide fragments having at least six amino acids in length, more preferably at least 9 or more amino acids in length, derived from any isoform of myelin proteolipid protein, such as

(SEQ ID NO: 266)
MGLLECCARCLVGAPFASLVATGLCFFGVALFCGCGHEALTGTEKLIET
YFSKNYQDYEYLINVIHAFQYVIYGTASFFFLYGALLLAEGFYTTGAVR
QIFGDYKTTICGKGLSATVTGGQKGRGSRGQHQAHSLERVCHCLGKWLG
HPDKFVGITYALTVVWLLVFACSAVPVYIYFNTWTTCQSIAFPSKTSAS
IGSLCADARMYGVLPWNAFPGKVCGSNLLSICKTAEFQMTFHLFIAAFV
GAAATLVSLLTFMIAATYNFAVLKLMGRGTKF.

In some embodiments of tolerance vaccines used to treat multiple sclerosis or other CNS autoimmune diseases including neuromyelitis optica, the peptide antigen is selected from.

In some embodiments, the vaccine for inducing tolerance comprises peptide antigens selected from gliadin typically selected from QLQPFPQPELPYPQPYPQQPEQPYPQPQP (SEQ ID NO:267), QPYPQQPEQPYPQPQPQYSQPEQPISQQ (SEQ ID NO:268), QPYPFRPEQPYPQPQPQYSQPEQPISQQ (SEQ ID NO:269), PSGEGSFQPSQENPQAQGSVQPQQLPQF (SEQ ID NO:270), QQFPQPEQPFPQQPEQPFPQQPQ (SEQ ID NO:271), PQPQQPEQPFPQSQQPEQPFPQPEQ (SEQ ID NO:272), QPFPQPEQEFPQPQQPQQSFPEQQPSL (SEQ ID NO:273), PQPQQPFPEQPQQPFPEQPQ (SEQ ID NO:274), PQPEQPQQPFPQSEQPQQPFPQPEQ (SEQ ID NO:275), QPFPQPEQEFPQPQQPQQSFPEQEPSL (SEQ ID NO:276), PTPLQPEQPFPQQPQQPQQPFPQPEQPFPWQPQ (SEQ ID NO:277), QQPPFSEQEQPVLPQ (SEQ ID NO:278), QGQEGYYPTSPEQPG (SEQ ID NO:279), IAQQQPFPEQPQPYPEQPQPYPQQ (SEQ ID NO:280), PQQPFPQPEQPFPSQ (SEQ ID NO:281), PQQPFPQPEQPFPLQ (SEQ ID NO:282), QFPPQPEQPFPQPQQ (SEQ ID NO:283), QFPPQPEQPFPQPHQ (SEQ ID NO:284), PQQPIPEQPQPYPQQ (SEQ ID NO:285), SQQPIPEQPQPYPQQ (SEQ ID NO:286), QPFPQPEQEFPQPQQ (SEQ ID NO:287), QSIPQPEQPFPQPEQPFPQS (SEQ ID NO:288) and PQQPFPEQPEQIIPQ (SEQ ID NO:289).

In certain preferred embodiments of tolerance vaccines for treating multiple sclerosis, the tolerance vaccine comprises peptide antigens consisting of overlapping peptides derived from myelin oligodendrocyte glycoprotein, myelin basic protein and/or myelin proteolipid protein, wherein the overlapping peptides consist of 35 amino acid peptide antigens that have between 13 to 19 amino acid overlap with at least one other peptide antigen in the overlapping peptide pool. In some embodiments of tolerance vaccines for treating multiple sclerosis, the tolerance vaccine comprises peptide antigens derived from myelin oligodendrocyte glycoprotein, myelin basic protein and/or myelin proteolipid protein selected on the basis of having epitopes with predicted high binding affinity for MHC-I and/or MHC-II. A non-limiting example of a tolerance vaccine for treating multiple sclerosis comprising peptide antigens derived from myelin oligodendrocyte glycoprotein, myelin basic protein and/or myelin proteolipid that have predicted high binding affinity for MHC-II includes but is not limited to peptide antigens selected from SLPSCLCSFLLLLLLQVSSSYAGQFRVIGPRHPIR (SEQ ID NO:290), VSSSYAGQFRVIGPRHPIRALVGDEVELPCRISPG (SEQ ID NO:291), EEAAMELKVEDPFYWVSPGVLVLLAVLPVLLLQIT (SEQ ID NO:292), SPGVLVLLAVLPVLLLQITVGLIFLCLQYRLRGKL (SEQ ID NO:293), QITVGLIFLCLQYRLRGKLRAEIENLHRTFDPHFL (SEQ ID NO:294), GKLRAEIENLHRTFDPHFLRVPCWKITLFVIVPVL (SEQ ID NO:295), HFLRVPCWKITLFVIVPVLGPLVALIICYNWLHRR (SEQ ID NO:296), PVLGPLVALIICYNWLHRRLAGQFLEELRNPF (SEQ ID NO:297), DPGSRPHLIRLFSRDAPGREDNTFKDRPSESDELQ (SEQ ID NO:298), GRTQDENPVVHFFKNIVTPRTPPPSQGKGRGLSLS (SEQ ID NO:299), GGRASDYKSAHKGFKGVDAQGTLSKIFKLGGRDSR (SEQ ID NO:300), FSKNYQDYEYLINVIHAFQYVIYGTASFFFLYGAL (SEQ ID NO:301), AFQYVIYGTASFFFLYGALLLAEGFYTTGAVRQIF (SEQ ID NO:302), GALLLAEGFYTTGAVRQIFGDYKTTICGKGLSATV (SEQ ID NO:303), HPDKFVGITYALTVVWLLVFACSAVPVYIYFNTWT (SEQ ID NO:304), LLVFACSAVPVYIYFNTWTTCQSIAFPSKTSASIG (SEQ ID NO:305), TWTTCQSIAFPSKTSASIGSLCADARMYGVLPWNA (SEQ ID NO:306), SICKTAEFQMTFHLFIAAFVGAAATLVSLLTFMIA (SEQ ID NO:307), AAFVGAAATLVSLLTFMIAATYNFAVLKLMGRGTK (SEQ ID NO:308), MIAATYNFAVLKLMGRGTKF (SEQ ID NO:309) or any fragments thereof having at least 6 amino acids in length, preferably at least 9 amino acids, or derivatives thereof.

In some embodiments the antigen is derived from a protein involved in cardiovascular disease or neurodegenerative disease. Non-limiting examples of proteins involved in cardiovascular disease include full-length proteins or protein fragments or engineered non-natural epitopes based on PCSK9, ANGPTL3 and similar such proteins.

Non-limiting examples of peptides antigens derived from PCSK9 include the sequences RGYLTKILHVFHGLLPGFLVKMSGDLLE (SEQ ID NO: 106), PGFLVKMSGDLLE (SEQ ID NO:107), PGFLVKnSGDLLE (SEQ ID NO:108) (wherein n=norleucine), PGFLVKMSSDLLG (SEQ ID NO:109), PGFLVKnSSDLLG (SEQ ID NO:110) (wherein n=norleucine), SIPWNLERITPPR (SEQ ID NO: 111), SIPWNLERITPPR (SEQ ID NO: 112), SIPWNLE (SEQ ID NO:113), SIPWNLEKVTPPR (SEQ ID NO:114), SIPWNLDRVTPPR(SEQ ID NO:115), NVPEEDGTRFHRQASKC (SEQ ID NO:116), NVPEEDGTRFHRQASK (SEQ ID NO:117), PEEDGTR (SEQ ID NO:118), NVPEEDG (SEQ ID NO:119), NVPEEDATRFHRQGSK (SEQ ID NO:120), LFAPGEDIIGASSDCSTCFVSQSGTSQAAA (SEQ ID NO:121), CSTCFVSQSGTSQAAA (SEQ ID NO:122), STCFVSQSGTSQAAA (SEQ ID NO:123), STBFVSQSGTSQAAA (SEQ ID NO:124) (wherein B=alpha aminobutyric acid) and STBFVSQ (SEQ ID NO:125) (wherein B=alpha aminobutyric acid).

Non-limiting examples of peptides antigens derived from ANGPTL3 include the sequences MFTIKLLLFIVPLVISSRIDQDNSSFDSLSPEPKSRFAMLDDVKILANGLLQLGHGLKDFVHKT KGQIND (SEQ ID NO:126), EPKSRFAMLDDVKILANGLLQLGHGLKDFVHKTKGQIND (SEQ ID NO:127), EPKSRFAMLDDVKI (SEQ ID NO:128), MLDDVKILANGLLQ (SEQ ID NO:129), LANGLLQLGHGLKD (SEQ ID NO:130), LGHGLKDFVHKTKG (SEQ ID NO:131), LKDFVHKTKGQIND (SEQ ID NO: 132), RFAMLDDVKILANGLLQLGH (SEQ ID NO:133), GLLQLGHGLKDFVHKTKGQI (SEQ ID NO:134 and IFQKLNIFDQSFYDLSLQTSEIKEEEKELRRTTYKLQVKNEEVKNMSLELNSKLESLLEEKILLQ QKVKY (SEQ ID NO:318).

In some embodiments, the antigen is derived from a protein involved in neurodegenerative disease, including proteins or portions of proteins or derivatives thereof that are known to form amyloids, including β amyloid peptide, alpha synuclein and microtubule-associated protein Tau.

Non-limiting examples of peptide antigens derived from Tau include MAEPRQEFEVMEDHAGTY (SEQ ID NO:310), VQKEQAHSEEHLGRAAFPGAPG (SEQ ID NO:311), EDRDVDESSPQDSPPS (SEQ ID NO:312), GQDAPLEFTFHV (SEQ ID NO:313), MAEPRQEFEVMEDHAGTYGLGDRKD (SEQ ID NO:314), EVMEDHAGTYG (SEQ ID NO:315), MAEPRQEFEVMEDHAGTY (SEQ ID NO:316) and RKDQGGYTMHQDQEGDTDAGLKES (SEQ ID NO:317) or any fragments thereof having at least 6 amino acids in length, preferably at least 9 amino acids, or derivatives thereof.

In some embodiments the antigen is a toxin or drug of abuse, or an analog of a toxin or drug of abuse that results in antibodies directed against the toxin or drug of abuse. In some embodiments, the antigen is an organophosphate that induces antibodies against organophosphate nerve agents.

Those skilled in the art recognize that any peptide, protein or post-translationally modified protein (e.g., glycoprotein) that leads to an immune response and is useful in the prevention or treatment of a disease can be selected for use as a peptide antigen (A) for use in the immunogenic compositions of the present invention.

### Molar Ratio of Peptide Antigen Conjugate to Amphiphile

In preferred embodiments of immunogenic compositions comprising vaccines, the vaccines comprise particles that further comprise one or more peptide antigen conjugates (e.g., [S]-[E1]-A-[E2]-[U]-H) and an amphiphile (e.g., S-[B]-[U]-H). The amphiphile includes a solubilizing block (S) that functions to stabilize particles and account for hydrophobic properties of some peptide antigens (A). However, the molar ratio of the peptide antigen conjugate to amphiphile was found to affect the stability and hydrodynamic behavior of particles comprising peptide antigen conjugates and amphiphiles. Accordingly, peptide antigen conjugate to amphiphile molar ratios of between about 4:1 to 1:1,000 were found to be generally well-tolerated, i.e., particles comprising >20% amphiphile by molar amount were generally stable, whereas those with less than 20% amphiphile by molar amount tended to aggregate when the average net charge of the one or more peptide antigen conjugates was near neutral charge, e.g., between +5 to −5 average net charge, i.e., higher proportions of amphiphile were needed to stabilize particles comprising peptide antigen conjugates with average net charge near neutral, whereas less amphiphile or no amphiphile was needed to stabilize particles comprising peptide antigen conjugates with high average net positive charge greater than or equal to +6 or less or equal to −6. While higher proportions of amphiphiles tended to result in improved particle stability, there was an inverse relationship between amphiphile percent molar amount and immunogenicity, with higher amphiphile proportions generally leading to lower immunogenicity, possibly due to relatively lower peptide antigen (A) loading in particles. Therefore, the amphiphile proportion should be tuned to ensure particle stability without sacrificing biological activity. Thus, in preferred embodiments of vaccines that comprise an amphiphile, the peptide antigen conjugate to amphiphile molar ratio is typically selected from between about 4:1 to about 1:20, such as 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, though more preferably between about 2:1 to about 1:4, such as about 1:1.

For vaccines comprising peptide antigen conjugates with average GRAVY scores that are moderate, or high, e.g., GRAVY>0, or >1, respectively, the molar ratio of peptide antigen conjugate to amphiphile should generally be higher, such as between 2:1 to about 1:20, or more preferably about 1:1 to about 1:20, such as 1:1 to about 1:4. However, the net charge of the peptide antigen conjugate also contributes to particle stability and affects the amount of amphiphile that may be needed. For instance, for vaccines comprising peptide antigen conjugates with average net charge greater than or equal to +3 or less than or equal to −3, less amphiphile may be required to form nanoparticles of stable size. For such compositions, the molar ratio of peptide antigen conjugate to amphiphile is preferably between about 4:1 to about 1:4, more preferably between about 3:1 to 1:3 or between about 2:1 to 1:2, most preferably 1:1.

An unexpected finding disclosed herein is that vaccines comprising peptide antigen conjugates with average net charge greater than or equal to +3 further comprising amphiphiles with neutral or near neutral charge at molar ratios of peptide antigen conjugate to amphiphile between about 1:1 or lower (i.e., higher proportions of amphiphile) induced less hemolysis than compositions without amphiphile or lower proportions of amphiphile. These results suggest that the amphiphile can shield the hemolytic activity of peptide antigen conjugates with net positive charge and that the molar proportion and composition of amphiphile included in vaccine compositions can be controlled to mitigate hemolysis.

Similarly, it was observed that vaccines comprising peptide antigen conjugates with average net charge greater than or equal to +3 further comprising amphiphiles with negative charge at molar ratios of peptide antigen conjugate to amphiphile of about 1:1 or lower (i.e., higher proportion of amphiphile) induced less hemolysis than compositions without amphiphile or lower proportions of amphiphile, but that compositions with molar ratio of peptide antigen conjugate between 2:1 and 1:2 generally aggregated. Thus, for preferred compositions of vaccines wherein the average net charge of the peptide antigen conjugate is positive and the net charge of the amphiphile is negative, the molar ratio of peptide antigen conjugate to amphiphile should be selected to ensure an overall net negative charge. Therefore, in preferred embodiments of vaccines comprising peptide antigen conjugates with average net positive charge and amphiphile(s) with average net negative charge, the molar ratio of peptide antigen conjugate to amphiphile is 1:2 or less, typically between about 1:2 to 1:16, more preferably between about 1:2 to 1:6, such as 1:3 to 1:5 or about 1:4.

### Immunomodulatory Drug Molecules (D) for Use in Vaccine Compositions

Preferred compositions of vaccines include one or more immunomodulatory drug molecules selected from immunostimulants and/or immunosuppressants.

The selection of the immunomodulatory drug molecules for use in vaccines depends on the application. For compositions of vaccines for treating or preventing cancer (“cancer vaccines”), the one or more immunomodulatory drug molecules are typically selected from immunostimulants, more preferably immunostimulants that induce Type-I IFNs, including agonists of TLR-3, TLR-7, TLR-8, TLR-9, RLR and STING. For compositions of vaccines for treating or preventing infectious diseases (“infectious disease vaccines”), the one or more immunomodulatory drug molecules are typically selected from immunostimulants that induce proinflammatory cytokines and/or Type-I IFNs, including agonists of TLR-1, TLR-2, TLR-3, TLR-4, TLR-5, TLR-6, TLR-7, TLR-8, TLR-9, CLRs, NLRs or combinations thereof. For compositions of vaccines for inducing tolerance to treat allergies or autoimmune diseases, the one or more immunomodulatory drug molecules are typically selected from immunosuppressants such as Treg promoting immunomodulators (defined below) or a combination of Treg promoting immunomodulators and immunostimulants selected from agonists of TLR-1, TLR-2, TLR-3, TLR-4, TLR-5, TLR-6, TLR-7, TLR-8, TLR-9, CLRs, NLRs or combinations thereof. Preferred compositions and combinations of specific immunomodulatory drugs are described in greater detail in later sections.

Compositions of immunostimulants suitable for use in vaccines are typically selected from PRR agonists. Non-limiting examples of pattern recognition receptor (PRR) agonists include TLR-1/2/6 agonists (e.g., lipopeptides and glycolipids, such as Pam2cys or Pam3cys lipopeptides); TLR-3 agonists (e.g., dsRNA, such as PolyI:C, and nucleotide base analogs); TLR-4 agonists (e.g., lipopolysaccharide (LPS) derivatives, for example, monophosphoryl lipid A (MPL) and small molecule derivatives or analogs of pyrimidoindole); TLR-5 agonists (e.g., Flagellin); TLR-7 & -8 agonists (e.g., ssRNA and nucleotide base analogs, including derivatives of imidazoquinolines, hydroxy-adenine, benzonahpthyridine and loxoribine); and TLR-9 agonists (e.g., unmethylated CpG); Stimulator of Interferon Genes (STING) agonists (e.g., cyclic dinucleotides, such as cyclic diadenylate monophosphate and diABZI or derivatives thereof); C-type lectin receptor (CLR) agonists (such as various mono, di, tri and polymeric sugars that can be linear or branched, e.g., mannose, Lewis-X trisaccharides, etc.); RIG-I-like receptor (RLR) agonists; NOD-like receptor (NLR) agonists (such as peptidogylcans and structural motifs from bacteria, e.g., meso-diaminopimelic acid and muramyl dipeptide); and combinations thereof. In some embodiments, the immunostimulant selected for use in a vaccine is selected from inorganic salts, including aluminum salts and or oils, such as squalene and its derivatives (e.g., MF59 and the like).

In several embodiments of vaccines, the vaccine comprises an immunostimulant selected from a TLR agonist, such as an imidazoquinoline-based TLR-7/8 agonist. For example, the immunostimulant can be Imiquimod (R2137) or Resiquimod (R2148), which are approved by the FDA for human use for certain indications and uses.

In several embodiments of vaccines, the vaccine comprises a TLR-7 agonist, a TLR-8 agonist and/or a TLR-7/8 agonist. Numerous such agonists are known, including many different imidazoquinoline compounds.

Imidazoquinolines are of use in the methods disclosed herein. Imidazoquinolines are synthetic immunomodulatory drugs that act by binding Toll-like receptors -7 and/or -8 (TLR-7/TLR-8) on antigen presenting cells (e.g., dendritic cells), structurally mimicking these receptors' natural ligand, viral single-stranded RNA. Imidazoquinolines are heterocyclic compounds comprising a fused quinoline-imidazole skeleton. Derivatives, salts (including hydrates, solvates, and N-oxides), and prodrugs thereof also are contemplated by the present disclosure. Particular imidazoquinoline compounds are known in the art, see for example, U.S. Pat. Nos. 6,518,265; and 4,689,338. In some non-limiting embodiments, the imidazoquinoline compound is not imiquimod or resiquimod.

In some embodiments, the immunostimulant is a small molecule having a 2-aminopyridine fused to a five membered nitrogen-containing heterocyclic ring, including but not limited to imidazoquinoline amines and substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, hydroxylamine substituted imidazoquinoline amines, oxime substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, hydroxylamine substituted tetrahydroimidazoquinoline amines, oxime substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; pyrazolopyridine amines; pyrazoloquinoline amines; tetrahydropyrazoloquinoline amines; pyrazolonaphthyridine amines; tetrahydropyrazolonaphthyridine amines; and 1H-imidazo dimers fused to pyridine amines, quinoline amines, tetrahydroquinoline amines, naphthyridine amines, or tetrahydronaphthyridine amines.

In some embodiments, the immunostimulant is an imidazoquinoline with the formula:

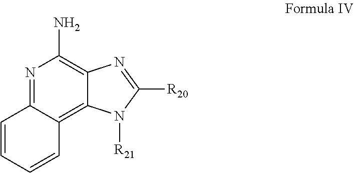

In Formula IV, R²⁰ is selected from one of hydrogen, optionally-substituted lower alkyl, or optionally-substituted lower ether; and R²¹ is selected from one of optionally substituted arylamine, or optionally substituted lower alkylamine. R²¹ may be optionally substituted to a linker that links to a polymer. An unexpected finding was that in some compounds wherein R²¹ was selected from a lower alkylamine, while the compound was less potent than R²¹ selected from an arylamine, the quality of response was improved. Thus, moderate potency Adjuvants of Formula IV led to better quality responses. Note: Adjuvant(s) of Formula IV are a type of Ligand and may be referred to as Adjuvants of Formula IV or Ligands with adjuvant properties.

In some embodiments, the R²⁰ included in Formula IV can be selected from hydrogen,

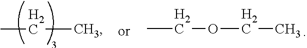

In some embodiments, the R²¹ can be selected from,

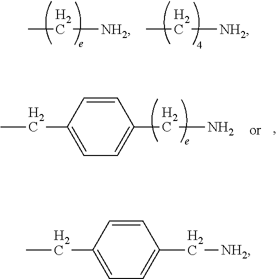

wherein e denotes the number of methylene unites is an integer from 1 to 4.

In some embodiments, R²¹ can be

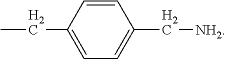

In some embodiments, R²¹ can be

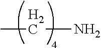

In some embodiments, R²⁰ can be

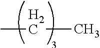

### and R²¹ can be

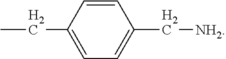

In some embodiments, at least one D is

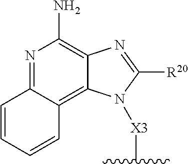

wherein R²⁰ is selected from H, alkyl, alkoxyalkyl, aryl, heteroaryl, aminoalkyl, amide and ester; and X3 is selected from alkyl, alkoxyalkyl, aralkyl, heteroaralkyl, aryl, heteroaryl and carboxy.

In some embodiments, wherein, R²⁰ is selected from H, alkyl and alkoxyalkyl; and X3 is selected from alkyl and aralkyl. In other embodiments, R²⁰ is butyl.

In some embodiments, X3 is alkyl.

In preferred embodiments of vaccines, the vaccine comprises nanoparticles further comprising amphiphiles, one or more peptide antigen conjugates and immunomodulatory drug molecules. In such embodiments, the immunomodulatory drug molecules may be incorporated into the nanoparticles through any suitable means.

In some embodiments, immunomodulatory drugs molecules that are hydrophobic and/or amphiphilic are incorporated into the nanoparticles comprising amphiphiles and one or more peptide antigen conjugates through non-covalent interactions, such as hydrophobic interactions with the hydrophobic blocks comprising the core of the nanoparticles. Non-limiting examples include, squalene-based immunostimulants; lipid-based PRR agonists, such as mincle receptor agonists (e.g., trehalose dimycolate and trehalose dibehenate) lipopolysaccharide-based agonists of TLR-4, and lipopeptide-based agonists of TLR-1/2 and TLR-2/6; heteroaryl-based agonists of TLR-4 (e.g., pyrimidoindole); agonists of TLR-7/8 (e.g., imidazoquinolines and benzonaphthyridines) and STING (e.g., diABZI); and various hydrophobic immunosuppressants, including but not limited to certain inhibitors of mTOR/PI3K/AKT (e.g., KU-0062794, Torin 1, Torin 2, etc.), CDK8/19 (e.g., Cortistatin), retinoic acid-related orphan gamma t (RORγt) (e.g., SR1555) and histone deacetylase (HDACs) (e.g., TMP269), as well as certain agonists of aryl hydrocarbon receptors (AHR) (e.g., indole, indolo[3,2-b]carbazole (ICZ) and 3,3 diindolomethane), retinoic acid receptors (RAR) (e.g., all-trans retinoic acid, TTNPB (cas: 71441-28-6), AM580, BMS753, BMS961 and the like) and adenosine receptor (e.g., UK-432,097).

In some embodiments, immunomodulatory drugs molecules are linked to hydrophobic blocks to form drug molecule conjugates that are incorporated into nanoparticles comprising amphiphiles and one or more peptide antigen conjugates through non-covalent interactions. In still other embodiments, immunomodulatory drugs molecules are incorporated into the nanoparticles comprising amphiphiles and one or more peptide antigen conjugates through covalent attachment to the amphiphiles and/or peptide antigen conjugates. In preferred embodiments of vaccines for cancer and infectious diseases, the vaccines comprise nanoparticles that comprise amphiphiles, one or more peptide antigen conjugates and immunostimulants selected from imidazoquinolines, wherein the imidazoquinolines are linked to the hydrophobic blocks of the amphiphiles and/or peptide antigen conjugates. Preferred compositions of vaccines are described in greater detail elsewhere.

### Compositions of Amphiphiles and Peptide Antigen Conjugates for Use in Vaccines

In preferred embodiments of vaccines, the vaccine comprises nanoparticles comprising one or more peptide antigen conjugates. In some embodiments, the vaccine further comprises an amphiphile and/or one or more immunomodulatory drug molecules.

The one or more peptide antigen conjugates included in vaccine compositions are selected to ensure that an adequate immune response can be induced in each subject. In preferred embodiments, vaccines typically include up to about 40 peptide antigens conjugates each comprising a unique antigen composition, though, typically no more than about 100 unique peptide antigen conjugates. Each peptide antigen conjugate comprises a unique peptide antigen (A) that comprises one or more known or predicted T cell epitopes (e.g., CD4 and/or CD8 T cell epitopes) and/or B cell epitopes. In general, vaccines for cancer treatment (“cancer vaccines”) include peptide antigen conjugates further comprising antigens with CD4 and/or CD8 T cell epitopes derived from tumor or viral antigens; vaccines for inducing tolerance (“tolerance vaccines”) include peptide antigen conjugates further comprising antigens with CD4 and/or CD8 T cell epitopes derived from autoantigens or allergens; and vaccines for inducing antibodies (“antibody vaccines) include peptide antigen conjugates further comprising antigens with one or more B cell epitopes derived from a target protein or include hapten conjugates (defined elsewhere) comprising a hapten that is typically derived from or a chemical analog of a toxin. Additionally, in preferred compositions of cancer vaccines and vaccines for inducing antibodies typically, the vaccine typically comprises cancer at least one or more additional peptide antigen conjugates comprising antigens selected from infectious disease antigens (e.g. flu antigens) and/or non-natural CD4 helper peptides, such as PADRE (e.g., AKFVAAWTLKAAA and related peptide sequences, e.g., wherein F is replaced with cyclohexylalanine), which function to enhance the response against the tumor antigens through, e.g., inducing CD4 T cell responses. A more detailed process for selecting antigens to include in peptide antigen conjugates, including the appropriate length and chemical composition is described in greater detail elsewhere.

In some embodiments of vaccines, the one or more peptide antigen conjugates comprise a solubilizing block (e.g., the peptide antigen conjugates have formula S-[E1]-A-[E2]-[U]-H-[D] or H[(D)]-U-[E1]-A-[E2]-S), which may function to improve solubility of the peptide antigen conjugate during manufacturing and/or to promote nanoparticle micellization when solubilized in an aqueous solution. In preferred compositions of vaccines that do not include an amphiphilic carrier molecule (e.g., S-[B]-[U]-H[(D)]), the one or more peptide antigen conjugates are typically selected from those comprising solubilizing blocks; and for vaccines that to include an amphiphilic carrier molecule, the one or more peptide antigens are typically selected from those that lack a solubilizing block.

In some embodiments of vaccines, the one or more peptide antigen conjugates comprise an N-terminal and/or C-terminal extension. In preferred compositions of vaccines, an extension is included between the hydrophobic block and the antigen, either directly or via a linker, e.g., A-E2-[U]-H-[D] or H-[D]-[U]-E1-A, and an extension is typically included between the solubilizing block and antigen if the solubilizing block is present, e.g., S-E1-A-[E2]-[U]-H-[D] or H-[D]-U-[E1]-A-E2-S. Preferred compositions and uses of extensions are described in greater detail elsewhere.

Peptide antigen conjugates may be manufactured by any suitable means. Typically, peptide antigen conjugates are either manufactured entirely on-resin by solid-phase peptide synthesis (SPPS) or are manufactured by convergent assembly of a peptide antigen fragment produced by SPPS and a hydrophobic block fragment, wherein the coupling of the peptide antigen fragment and hydrophobic block fragment may occur in solution or on-resin. The preference for the manufacturing process typically depends on the composition of the hydrophobic block. For instance, wherein the hydrophobic block of the peptide antigen conjugate comprises amino acids selected from tryptophan (and any analogs or derivatives thereof), para-aminophenylalanine, glutamic acid (any derivatives thereof), lysine or ornithine (and any derivatives thereof) and the like, the peptide antigen conjugate is typically produced entirely by SPPS. Wherein the hydrophobic block comprises drug molecules, particularly drug molecules that are of relatively high cost (relative to standard amino acid costs), the peptide antigen conjugate is typically manufactured by convergent assembly of two separately produced components (i.e., [S]-[E1]-A-[E2]-U1+U2-H-[D]) using linker chemistries described in greater detail elsewhere.

The incorporation of peptide antigen conjugates in nanoparticles of uniform size and composition is critical to ensuring consistent manufacturing and reliable induction of immune responses in subjects. The inventors of the present disclosure previously reported (see: Lynn et al. Nat. Biotech. 2020) that incorporation of one or more charged amino acid residues (referred to as charge-modifying groups) directly or indirectly via an extension to peptide antigens linked to hydrophobic blocks resulted in amphiphilic peptide antigen conjugates that self-assembled into uniform nanoparticle micelles if the peptide antigen conjugate had appropriate net charge. Potential limitations of this approach (i.e., incorporating a solubilizing block onto each peptide antigen conjugate) are that it is generally preferred to manufacture charge-modifying groups onto each antigen during SPPS to reduce manufacturing complexity and costs, but the reliance on SPPS for introducing the SPPS can limit the scope of chemical compositions that are practicable. For instance, it was found that net positive charge-modifying groups based on lysine, but not net negative charge-modifying groups based on glutamic acid, aspartic acid, or phosphoserine, could be readily incorporated into any peptide antigen during manufacturing by SPPS, thus limiting the scope of potential charge-modifying groups that could be practically utilized.

To overcome the challenges associated with incorporating solubilizing blocks directly onto peptide antigen conjugates, the inventors of the present disclosure developed novel amphiphilic carrier molecules of formula S-[B]-[U]-H-[D] to use in combination with the peptide antigen conjugates. This enabled the evaluation of a greater breadth of possible solubilizing block compositions and architectures that could be included in immunogenic compositions, e.g., vaccines comprising at least one peptide antigen conjugate. Indeed, the authors of the present disclosure synthesized and evaluated amphiphiles of formula S-[B]-[U]-H-[D] with varying architecture (linear, cone and brush), charge (net positive, net negative or net neutral charge), chemical composition and size (e.g., length of linear polymers or oligomers, or generation of dendrons) and evaluated how various properties of the amphiphile combined with one or more peptide antigen conjugates (at a range of molar ratios of amphiphile to peptide antigen conjugate) impact hydrodynamic behavior and immunogenicity in vivo.

An unexpected finding was that peptide antigen conjugates admixed with amphiphiles with linear and cone (or “dendron”) architecture formed stable nanoparticle micelles with greater consistency and with up to higher peptide antigen conjugate to amphiphile ratios as compared with amphiphiles having brush architecture, provided that the amphiphiles with linear architecture had net charge≥+4 or net charge≤−4, or contained at least two or more sugar molecules, preferably between 2 to 8 or more sugar molecules (i.e., 2 to 8 or more monosaccharides as either individual monosaccharides or more complex structures, e.g., 1 to 4 or more disaccharides). In contrast to the amphiphiles with linear architecture requiring net positive or net negative charge, or high sugar molecule content, the amphiphiles with cone architecture were found to form stable nanoparticle micelles independent of charge, i.e., stable nanoparticle formation was observed with amphiphiles with cone architecture having net positive, net negative and neutral charge at ratios of peptide antigen conjugate to amphiphile ranging from 4:1 to 1:1,000, though, higher ratios (i.e., greater proportions of peptide antigen conjugate) could be achieved when the peptide antigen conjugates comprise a solubilizing block and have average net charge greater than or equal to +6 or less than or equal to −6

Importantly, the ability of dendrons to enable nanoparticle micellization independent of net charge allowed for a thorough investigation of how different solubilizing groups impact vaccine formulation properties as well as biological activity. Accordingly, amphiphiles with cone architecture generally required up to 4 or more solubilizing groups, (e.g., saccharides, amines, carboxylic acids or hydroxyls) to ensure stable nanoparticle formation when combined with peptide antigen conjugates at molar ratios of 4:1 or less of peptide antigen conjugate to amphiphile. Thus, in preferred embodiments of amphiphiles with cone architecture, the amphiphile comprises 4 or more solubilizing groups, though, typically no more than 16 or 32 solubilizing groups per amphiphile. While amphiphiles having cone architecture and 4 or more solubilizing groups generally formed stable nanoparticle micelles with high ratios of peptide antigen conjugate to amphiphile, the specific chemical composition of the dendron amplifier, spacer and hydrophobic block also had an impact on hydrodynamic behavior. Accordingly, generation 2 to 4 PEG-based dendron amplifiers and PEG-based spacers (B) with between 4 to 36 monomeric units were found to lead to more uniform nanoparticle compositions as compared with amphiphiles with cone architecture comprising similar generation and length of peptide-based amplifying linkers and peptide-based spacers, respectively. Moreover, vaccines comprising amphiphiles with PEG-based spacers (B) with greater than 48 monomeric units tended to lead to less stable nanoparticle micelles and reduced immunogenicity than vaccines comprising amphiphiles with short PEG-spacers. However, the spacer length selected for the amphiphile also impacted the capacity of the amphiphile to prevent hemolysis when the vaccine composition further comprised peptide antigen conjugates with average net positive charge. Accordingly, amphiphiles with PEG spacers greater than or equal to 12 monomeric units were found to mitigate hemolysis more effectively than amphiphiles with shorter spacers.

Therefore, in certain preferred embodiments of vaccines, the vaccine comprises one or more, typically between 1 to 40, peptide antigen conjugates of formula [S]-[E1]-A-[E2]-[U]-H-[D] or H[(D)]-U-[E1]-A-[E2]-[S] and an amphiphile of formula S-B-[U]-H-[D] with cone architecture, wherein the amphiphile with cone architecture further comprises a solubilizing block comprising a PEG-based dendron with between 4 to 16 solubilizing groups and a PEG-based spacer with between 4 and 48 monomer units, more preferably 4 to 36 monomer units, most preferably 24 monomers units, additionally wherein the solubilizing groups comprise sugar molecules, carboxylic acids, amines and/or hydroxyls, and the hydrophobic block comprises a poly(amino acid) of Formula I. A non-limiting example is provided here for clarity:

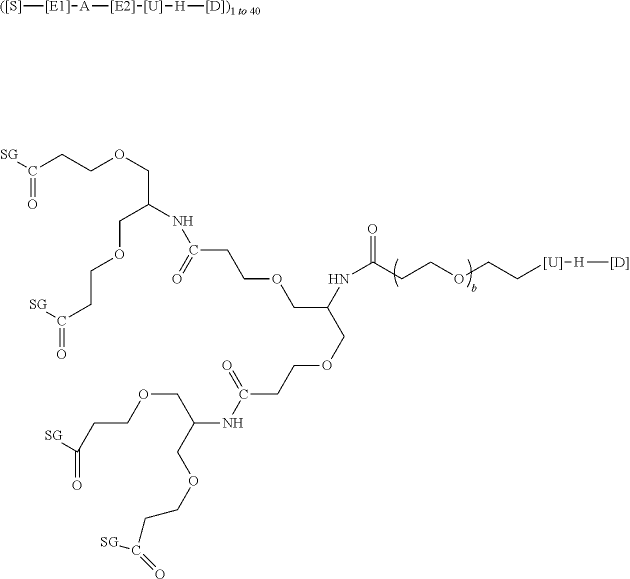

wherein b is an integer number of monomeric units comprising the spacer and is typically between 4 and 48, such as 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 22, 43, 44, 45, 46, 47 or 48 monomeric units, preferably between about 4 and 36 monomer units, most preferably 24 monomeric units; SG is selected from sugar molecules, carboxylic acids, amines and/or hydroxyls that are linked to S either directly or via a suitable linker X, or, more preferably, X5; the hydrophobic block (H) is typically selected from poly(amino acids) of Formula I; S is a solubilizing block; E1 is a N-terminal extension; A is an antigen; E2 is a C-terminal extension; U is a linker; D is drug molecule; and [ ] denotes that the groups are optional. In some alternative embodiments the peptide antigen conjugates have the formula H-[U]-[E1]-A-[E2]-[S]. For clarity, each occurrence of any of the components of vaccines described herein, e.g., H, S, A, E1, E2, B, D and any linkers (e.g., U) are independently selected.

While the intended used of the vaccine should be considered in selecting the composition of the solubilizing groups, the inventors of the present disclosure identified that for vaccines comprising one or more peptide antigen conjugates of formula [S]-[E1]-A-[E2]-[U]-H-[D] or H-[D]-U-[E1]-A-[E2]-[S] and an amphiphile of formula S-B-[U]-H-[D] with cone architecture, the solubilizing groups of the solubilizing block of the amphiphile selected from mannose generally led to higher magnitude T cell responses as compared with the use of amphiphiles with solubilizing groups comprising carboxylic acids, amines or hydroxyls. A non-limiting example of a vaccine comprising one or more, typically between 1 to 40, peptide antigen conjugates of formula [S]-[E1]-A-[E2]-[U]-H-[D] and an amphiphile of formula S-B-[U]-H-[D] with cone architecture, wherein the amphiphile with cone architecture further comprises a solubilizing block comprising a PEG-based dendron with 4 solubilizing groups (SG) and a PEG-based spacer with between 4 and 36 monomer units, additionally wherein the solubilizing groups comprise sugar molecules selected from mannose and the hydrophobic block comprises a poly(amino acid) of Formula I, is provided here for clarity:

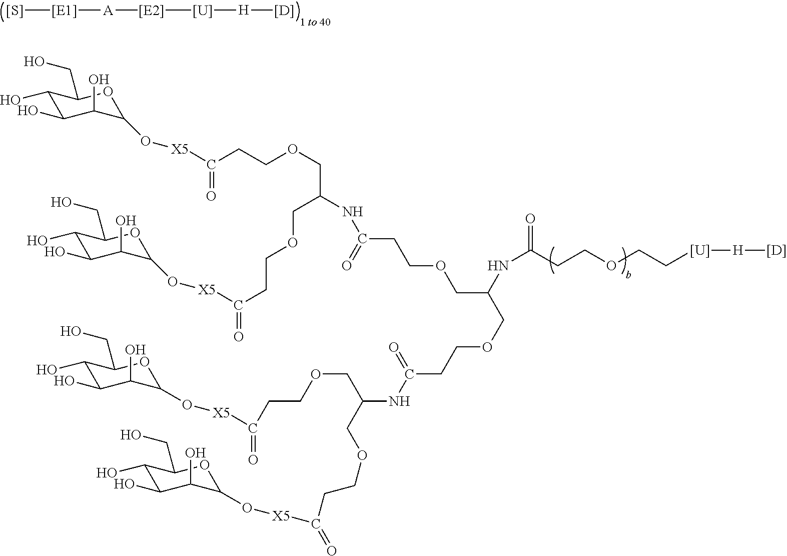

X5 is a suitable linker; b is an integer number of monomeric units comprising the spacer and is preferably between 4 and 36, such as 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35 or 36 monomeric units; the hydrophobic block (H) comprises a poly(amino acid) of Formula I; S is a solubilizing block, E1 is a N-terminal extension, A is an antigen, E2 is a C-terminal extension, U is a linker, D is a drug molecule and [ ] denotes that the groups are optional. In some alternative embodiments, the peptide antigen conjugates have the formula H-[U]-[E1]-A-[E2]-[S].

The intended use of the vaccine should also be considered when selecting suitable peptide antigen conjugate compositions as well as the hydrophobic block composition for both the peptide antigen conjugate and the amphiphile (if present), yet the authors of the present identified certain features of peptide antigen conjugates and hydrophobic block compositions that were generally preferred across different vaccine applications.

Accordingly, for vaccines comprising preferred compositions of amphiphilic carriers of formula S-[B]-[U]-H-[D] and one or more peptide antigen conjugates, it was found that the peptide antigens (A) could be linked directly or indirectly via an extension and or Linker U to a hydrophobic block (H) without use of a solubilizing block (S) (e.g., H-[D]-U-[E1]-A or A-[E2]-[U]-H[(D)]) and the resulting formulations of amphiphile and at least one peptide antigen conjugate generally led to nanoparticle micelles with consistent size particles that were stable over time and immunogenic in vivo.

However, it was also observed that vaccines comprising peptide antigen conjugates with net positive charge and solubilizing blocks selected from poly(amino acids) with positively charged groups, e.g., lysine and ornithine, generally had improved manufacturability and formulation properties (e.g., particle size uniformity and stability) as compared with vaccines comprising peptide antigen conjugates lacking solubilizing groups or having solubilizing groups with net negative charge. Therefore, in preferred embodiments of vaccines described herein the peptide antigen conjugate has net positive charge and comprises a solubilizing block further comprising poly(amino acids) with positively charged groups.

An additional notable finding was that the manufacturing process of certain peptide antigen conjugates could be further simplified based on the composition of the hydrophobic block (H). Accordingly, it was observed that for peptide antigen conjugates comprising hydrophobic blocks based on poly(amino acids) of Formula I comprising hydrophobic monomers M with aromatic groups further comprising aryl, heteroaryl, aminoaryl and/or aminoheteroaryl groups, and optionally charged amino acids (P) comprising amines, the entire peptide antigen conjugate could be produced by SPPS. Thus, in certain preferred embodiments of vaccines, the peptide antigen conjugate is selected from peptide antigen conjugates of formula H-[D]-[E1]-A-[E2]-[S] or [S]-[E1]-A-[E2]-H-[D] and the hydrophobic block of the peptide antigen conjugate comprises hydrophobic monomers M with aryl, heteroaryl, aminoaryl and/or aminoheteroaryl groups, and optionally charged amino acids (P) comprising amines, wherein the number of amino acids comprising the hydrophobic block is typically between 3 to 30.

Additionally, it was observed that peptide antigen conjugates and amphiphiles with hydrophobic blocks selected from poly(amino acids) of Formula I comprising hydrophobic monomers M with aryl, heteroaryl, aminoaryl and/or aminoheteroaryl groups improved manufacturability and led to more consistent nanoparticle formulations than those with hydrophobic blocks comprising higher alkanes or aromatic groups lacking nitrogen. Thus, in preferred embodiments of vaccines, the vaccine comprises one or more, typically between 1 to 40, peptide antigen conjugates of formula [S]-[E1]-A-[E2]-[U]-H-[D] or [D]-U-[E1]-A-[E2]-[S], and an amphiphile of formula S-B-[U]-H-[D] with cone architecture, wherein the amphiphile with cone architecture further comprises a solubilizing block comprising a PEG-based dendron with 4 solubilizing groups (SG) and a PEG-based spacer with between 4 and 36 monomer units, additionally wherein the solubilizing groups comprise sugar molecules selected from mannose and the hydrophobic block of both the peptide antigen conjugate and the amphiphile comprises a poly(amino acid) of Formula I comprising hydrophobic monomers, M, with aryl, heteroaryl, aminoaryl and/or aminoheteroaryl groups:

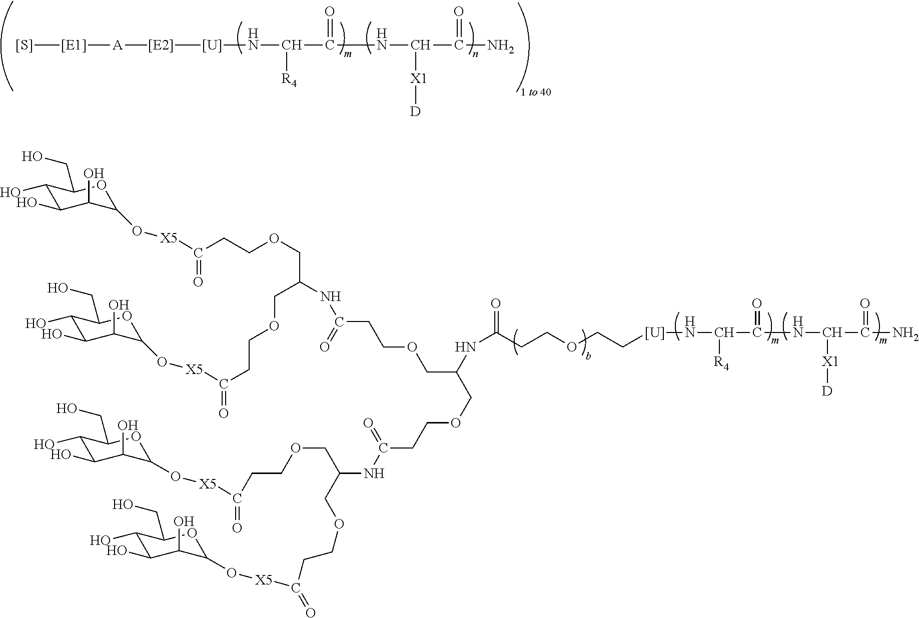

wherein X5 is a suitable linker; b is an integer number of monomeric units comprising the spacer and is typically between 4 and 36, such as 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35 or 36 monomeric units; the hydrophobic block (H) comprises a poly(amino acid) of Formula I, wherein R⁴ is selected from aryl, heteroaryl, aminoaryl and/or aminoheteroaryl groups and m is typically between 3 and 30; A is an antigen, S is a solubilizing block, E1 is a N-terminal extension, E2 is a C-terminal extension, U is a linker and [ ] denotes that the groups are optional. In some alternative embodiments the peptide antigen conjugates have the formula H-[U]-[E1]-A-[E2]-[S].

In the above example, wherein the hydrophobic monomer is para-aminophenylalanine (sometimes abbreviated “F’) the structures of the peptide antigen conjugate and amphiphile are:

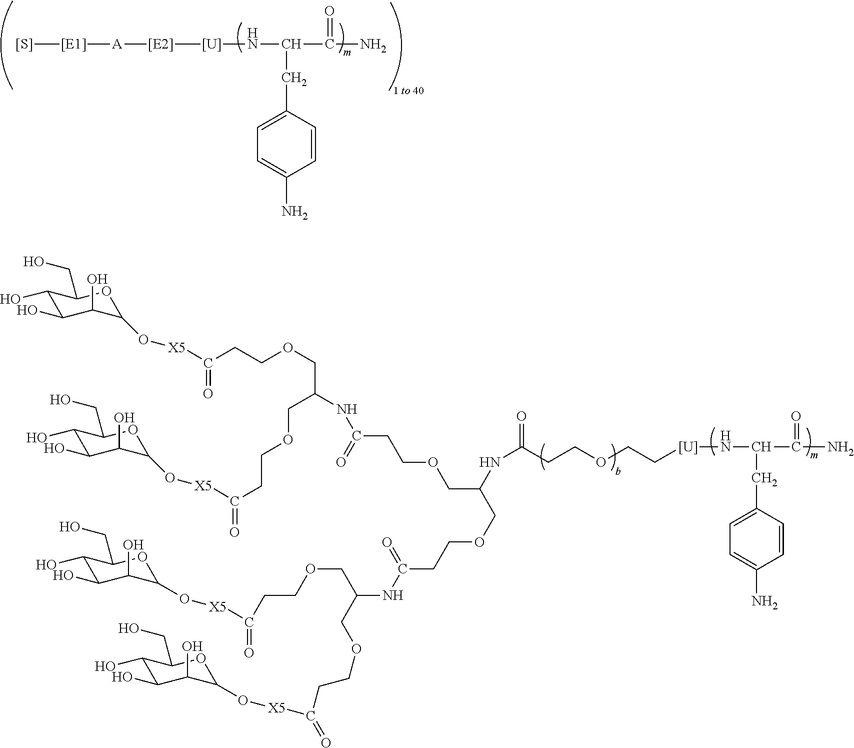

In certain preferred embodiments of vaccines, a drug molecule is included in the hydrophobic block of the peptide antigen conjugate and/or amphiphile. A non-limiting example is provided here for clarity:

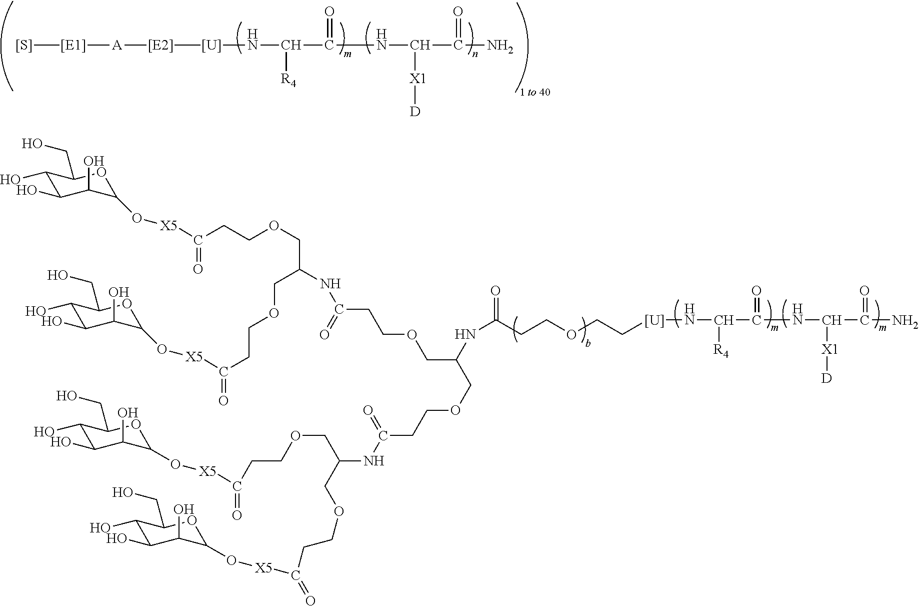

wherein X1 and X5 are suitable linkers; b is an integer number of monomeric units comprising the spacer and is typically between 4 and 36, such as 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35 or 36 monomeric units; the hydrophobic block (H) comprises a poly(amino acid) of Formula I, wherein R₄ is selected from aryl, heteroaryl, aminoaryl and/or aminoheteroaryl groups; the drug (D) is any suitable immunomodulatory drug; m and n are an integer number of repeating units of monomers M and N, wherein the sum of m and n is typically between 3 and 30; A is an antigen, S is a solubilizing block, E1 is a N-terminal extension, E2 is a C-terminal extension, U is a linker and [ ] denotes that the groups are optional. In some alternative embodiments the peptide antigen conjugates have the formula H-[U]-[E1]-A-[E2]-[S].

In preferred embodiments of vaccines for cancer treatment and inducing antibodies, and in some compositions of vaccines for inducing tolerance that further comprise drug molecules selected from inhibitors of mTOR, the vaccine comprises drug molecules selected from imidazoquinolines that are covalently linked to the hydrophobic block of the peptide antigen conjugate and/or amphiphile. A non-limiting example is shown here for clarity:

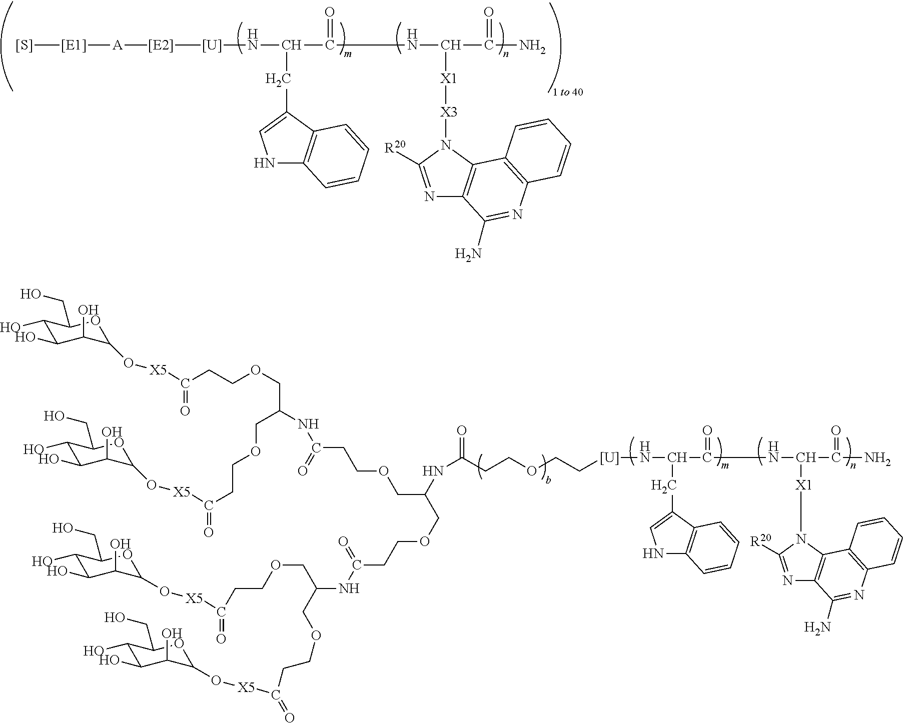

In preferred embodiments of vaccines wherein the hydrophobic block of the peptide antigen conjugate and/or amphiphile comprises a drug molecule, the hydrophobic block is linked to the antigen and amphiphile through a Linker U comprising a triazole. A non-limiting example is provided here for clarity:

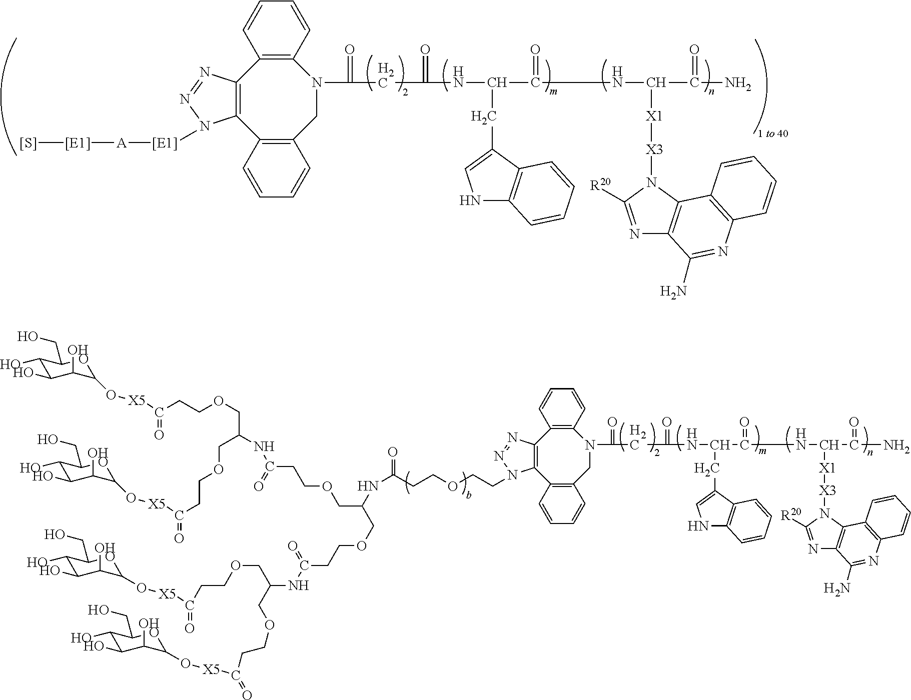

For certain preferred embodiments of vaccines, particularly for personalized vaccines, the inventors of the present disclosure found it preferable to use different hydrophobic block compositions for the peptide antigen conjugate and the amphiphile. For instance, for vaccines wherein a unique set of peptide antigen conjugates is provided to each patient, the inventors of the present disclosure found that it was preferred to use conjugates with hydrophobic blocks comprising poly(amino acids) of Formula I further comprising hydrophobic monomers, M, with aryl, heteroaryl, aminoaryl and/or aminoheteroaryl groups, and optionally charged amino acids (P) comprising amines, wherein the number of amino acids comprising the hydrophobic block is typically between 3 to 30; and to use amphiphiles with hydrophobic blocks comprising drug molecules. A non-limiting example is provided here for clarity:

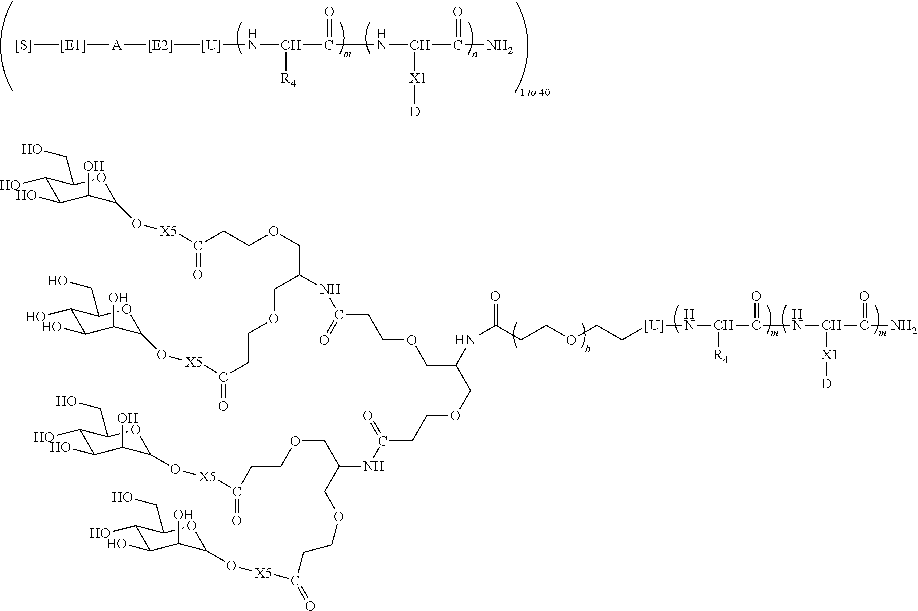

wherein X1 and X5 are each independently any suitable linker; b is an integer number of monomeric units comprising the spacer and is typically between 4 and 36, such as 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35 or 36 monomeric units; the hydrophobic block of the peptide antigen conjugate comprises a poly(amino acid) of Formula I, wherein R4 is selected from aryl, heteroaryl, aminoaryl and/or aminoheteroaryl groups and m is typically between 3 and 30; the hydrophobic block of the amphiphile comprises a poly(amino acid) of Formula I, wherein R₄ is selected from aryl, heteroaryl, aminoaryl and/or aminoheteroaryl groups; the drug (D) is any suitable immunomodulatory drug; m and n are an integer number of repeating units of monomers M and N, wherein the sum of m and n is typically between 3 and 30; A is an antigen, S is a solubilizing block, E1 is a N-terminal extension, E2 is a C-terminal extension; U is a linker and [ ] denotes that the groups are optional. In some alternative embodiments the peptide antigen conjugates have the formula H-[U]-[E1]-A-[E2]-[S].

In still other embodiments, of vaccines for cancer treatment and inducing antibodies, and in some compositions of vaccines for inducing tolerance that further comprise drug molecules selected from inhibitors of mTOR, the vaccine comprises drug molecules selected from imidazoquinolines that are covalently linked to the hydrophobic block of the peptide antigen conjugate but not the amphiphile. A non-limiting example is shown here for clarity:

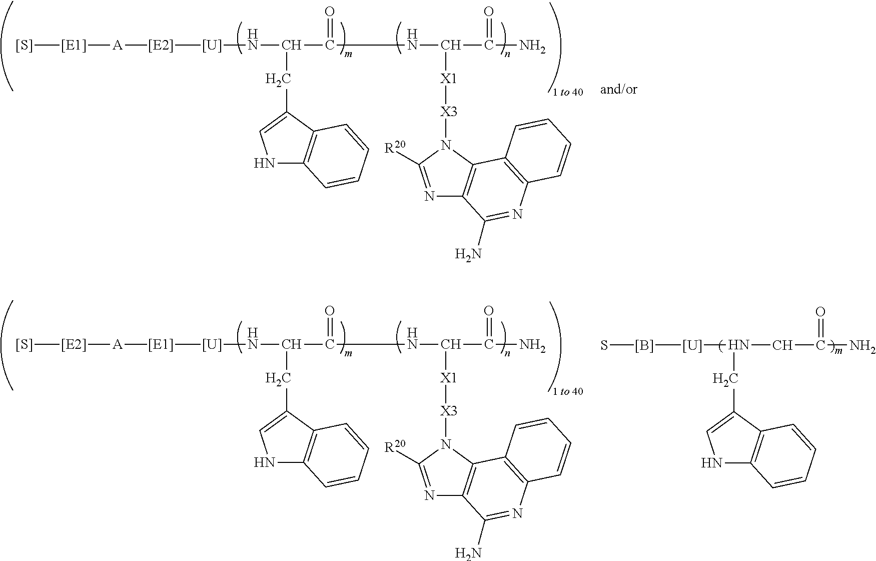

For example, wherein the amphiphile has dendron architecture and comprises a solubilizing block comprising a PEG-based dendron with 4 solubilizing groups (SG) and a PEG-based spacer with between 4 and 36 monomer units, additionally wherein the solubilizing groups comprise sugar molecules selected from mannose:

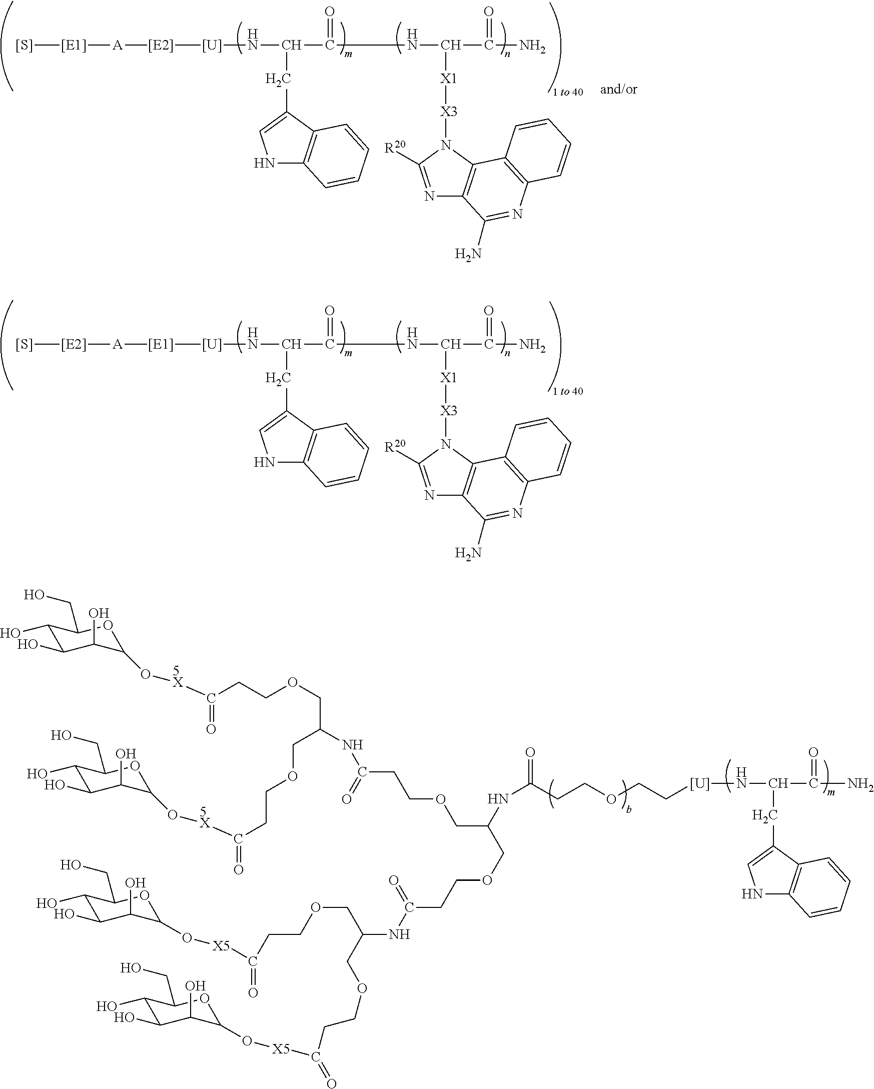

### Compositions of Vaccines for Preventing or Treating Cancer

Earlier sections provided general descriptions of vaccines, including compositions of peptide antigen conjugates, amphiphiles and drug molecules (including drug molecule conjugates) that are generally preferred for use in vaccines. This section describes specific, preferred embodiments of vaccines for preventing or treating cancer (“cancer vaccines”).

Cancer vaccines comprises nanoparticles comprising one or more peptide antigen conjugates. In preferred embodiments of cancer vaccines, the vaccine further comprises an amphiphile and an immunostimulatory drug molecule.

The one or more peptide antigen conjugates, typically between 1 and 40, each comprise an antigen (A), which is typically selected from tumor antigens, including self-antigens, neoantigens and viral antigens. In preferred compositions of cancer vaccines, at least one of the peptide antigen conjugates comprises an antigen (A) selected from tumor antigens, though, the cancer vaccine may also include one or more additional peptide antigen conjugates comprising antigens (A) selected from infectious disease antigens as well as non-natural CD4 helper peptides, such as PADRE. In some embodiments, the cancer vaccine comprises at least one peptide antigen conjugate comprising an antigen selected from neoantigens. In other embodiments, the cancer vaccine comprises at least one peptide antigen conjugate comprising an antigen selected from self-antigens. In other embodiments, the cancer vaccine comprises at least one peptide antigen conjugate comprising an antigen selected from viral antigens, more preferably viral antigens associated with a malignancy (e.g., HPV, HCV, polyoma virus, etc.).

The number of peptide antigen conjugates is selected to ensure that an adequate immune response can be induced in each subject. In preferred embodiments, vaccines for cancer treatment typically include up to about 40, though typically no more than 100, peptide antigen conjugates each comprising a unique peptide antigen (A) that comprises one or more CD4, CD8 T cell and/or B cell epitopes or predicted epitopes. A more detailed process for selecting antigens is described in greater detail elsewhere.

In preferred embodiments of cancer vaccines, the vaccine further comprises a drug molecule selected from immunostimulants that induce type-I IFNs and is typically selected from agonists of TLR-3, TLR-7, TLR-8, TLR-7/8a, TLR-9, RLR and STING. In preferred embodiments of cancer vaccines, the immunostimulant is selected from an imidazoquinoline of Formula IV, which is linked to the peptide antigen conjugates and/or amphiphile via a covalent bond.

In some embodiments of cancer vaccines, the one or more peptide antigen conjugates comprise a solubilizing block (e.g., the peptide antigen conjugates are selected from peptide antigen conjugates of formula S-[E1]-A-[E2]-[U]-H-[D] or H-[D]-U-[E1]-A-[E2]-S) and an amphiphilic carrier is not included. In preferred embodiments of cancer vaccines wherein the peptide antigen conjugates comprise a solubilizing block and the amphiphilic carrier (e.g., amphiphiles of formula S-[B]-[U]-H-[D]) is absent, the vaccine further comprises one or more drug molecules selected from immunostimulants that are included either admixed with the peptide antigen conjugates as the free drug molecule (e.g., D+S-[E1]-A-[E2]-[U]-H or H-U-[E1]-A-[E2]-S) or as a drug molecule conjugate (e.g., D-H+S-[E1]-A-[E2]-[U]-H-[D] or H-[D]-U-[E1]-A-[E2]-S), or the drug molecule is linked directly to the hydrophobic block of the peptide antigen conjugate (e.g., S-[E1]-A-[E2]-[U]-H-D or H-D-U-[E1]-A-[E2]-S). In certain preferred embodiments, the immunostimulant is selected from imidazoquinolines of Formula IV that are linked to the hydrophobic block through a covalent bond.

In other embodiments of cancer vaccines further comprising an amphiphilic carrier (e.g., an amphiphile of formula S-[B]-[U]-H-[D]), the one or more peptide antigen conjugates optionally include a solubilizing group (e.g., the peptide antigen conjugates are selected from peptide antigen conjugates of formula [S]-[E1]-A-[E2]-[U]-H-[D] or H-[D]-U-[E1]-A-[E2]-[S]). As described earlier in the disclosure, amphiphiles of formula S-B-[U]-H-D with cone architecture comprising solubilizing blocks comprising PEG-based dendrons with between 4 to 16 solubilizing groups and PEG-based spacers with between 4 and 36 monomer units, wherein the solubilizing groups comprise sugar molecules, carboxylic acids, amines and/or hydroxyls, and the hydrophobic block comprises a poly(amino acid) of Formula I, led to improved formulations with consistent and enhanced immunogenicity as compared with alternative amphiphile architectures and compositions. Though, solubilizing blocks comprising linear peptides with between 3 to 12 charged amino acids (e.g., lysine) and PEG-based spacers with between 4 and 36 monomer units, wherein the hydrophobic block comprises a poly(amino acid) of Formula I were also found to be suitable carriers for cancer vaccines.

Therefore, in preferred embodiments of cancer vaccines, the vaccine comprises one or more, typically between 1 to 40, peptide antigen conjugates of formula [S]-[E1]-A-[E2]-[U]-H-[D] or H-[D]-U-[E1]-A-[E2]-[S] and an amphiphile of formula S-B-[U]-H-[D] with cone architecture, wherein the amphiphile with cone architecture further comprises a solubilizing block comprising a PEG-based dendron with between 4 to 16 solubilizing groups and a PEG-based spacer with between 4 and 36 monomer units, additionally wherein the solubilizing groups comprise sugar molecules, carboxylic acids, amines and/or hydroxyls, and the hydrophobic block comprises a poly(amino acid) of Formula I. A non-limiting example is provided here for clarity:

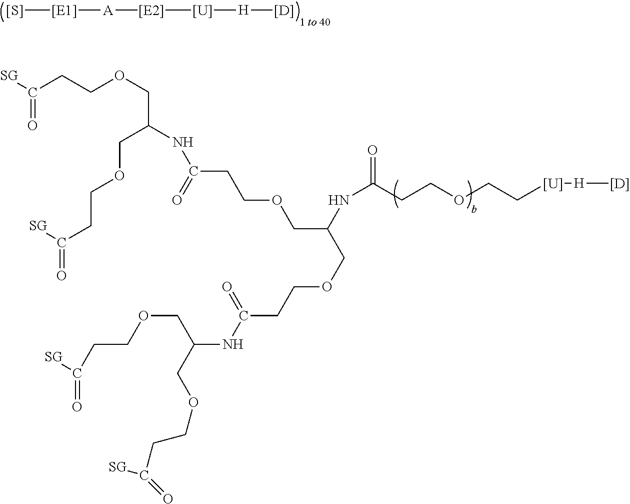

wherein b is an integer number of monomeric units comprising the spacer and is typically between 4 and 36, such as 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35 or 36 monomeric units; SG is selected from sugar molecules, carboxylic acids, amines and/or hydroxyls that are linked to S either directly or via a suitable linker X, or, more preferably, X5; the hydrophobic block (H) is typically selected from poly(amino acids) of Formula I; S is a solubilizing block, E1 is a N-terminal extension, A is an antigen selected from tumor antigens, E2 is a C-terminal extension, U is a linker, D is drug molecule and [ ] denotes that the groups are optional. In some alternative embodiments, the peptide antigen conjugates have the formula H-[U]-[E1]-A-[E2]-[S]. In preferred embodiments, the peptide antigen conjugate has net positive charge greater than or equal to +2, preferably between +3 and +5, and the solubilizing block of the peptide antigen conjugate is present and comprises a poly(amino acid) (or “peptide”) further comprising lysine and/or ornithine residues; the molar ratio of the peptide antigen conjugate to amphiphile is between 4:1 and 1:4, more preferably between 2:1 and 1:2 or about 1:1; and b comprises between 24 to 36 monomeric units.

In certain other preferred embodiments of cancer vaccines, the vaccine comprises one or more, typically between 1 to 40, peptide antigen conjugates of formula [S]-[E1]-A-[E2]-[U]-H-[D] or H-[D]-U-[E1]-A-[E2]-[S] and an amphiphile of formula S-B-[U]-H-[D] with linear architecture, wherein the amphiphile with linear architecture further comprises a solubilizing block comprising a peptide with between 3 to 12 charged amino acids and a PEG-based spacer with between 4 and 36 monomer units, and the hydrophobic block comprises a poly(amino acid) of Formula I.

In preferred embodiments of cancer vaccines, the vaccine comprises an immunostimulatory drug molecule and one or more, typically between 1 to 40, peptide antigen conjugates of formula [S]-[E1]-A-[E2]-[U]-H-D or H-[D]-U-[E1]-A-[E2]-[S] and an amphiphile of formula S-B-[U]-H-D with cone architecture, wherein the amphiphile with cone architecture further comprises a solubilizing block comprising a PEG-based dendron with between 4 to 16 solubilizing groups and a PEG-based spacer with between 4 and 36 monomer units, additionally wherein the solubilizing groups comprise sugar molecules selected from mannose or Sialyl Lewis^{x} (sLeX), and the hydrophobic block comprises a poly(amino acid) of Formula I further comprising an imidazoquinoline of Formula IV. A non-limiting example is provided here for clarity:

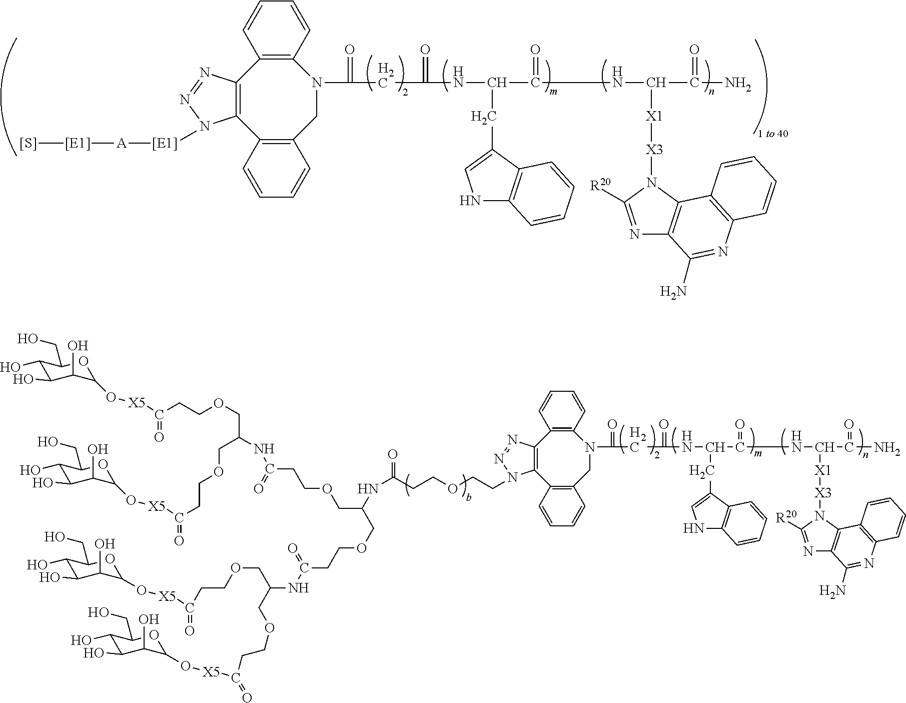

wherein X1, X3 and X5 are each independently any suitable linker molecule; b is an integer number of monomeric units comprising the spacer and is typically between 4 and 36, such as 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35 or 36 monomeric units; S is a solubilizing block, E1 is a N-terminal extension, E2 is a C-terminal extension, A is an antigen selected from tumor antigens, and [ ] denotes that the groups are optional. In some alternative embodiments the peptide antigen conjugates have the formula H-D-U-[E1]-A-[E2]-[S]. In preferred embodiments, the average net charge of the one or more peptide antigen conjugates is greater than or equal to +2, preferably between +2 and +6, more preferably between +3 and +5, and the solubilizing block of the peptide antigen conjugate is present and comprises a poly(amino acid) (or “peptide”) further comprising lysine and/or ornithine residues; the molar ratio of the peptide antigen conjugate to amphiphile is between 4:1 and 1:4, more preferably between 2:1 and 1:2 or about 1:1; and b comprises between 12 to 36 monomeric units, preferably 24 monomeric units.

For certain preferred compositions of cancer vaccines used as personalized on-demand therapies (“personalized cancer vaccines” or “PCVs”), drug molecules may be incorporated into the hydrophobic block of the amphiphile but not the hydrophobic block of the peptide antigen conjugate, such as:

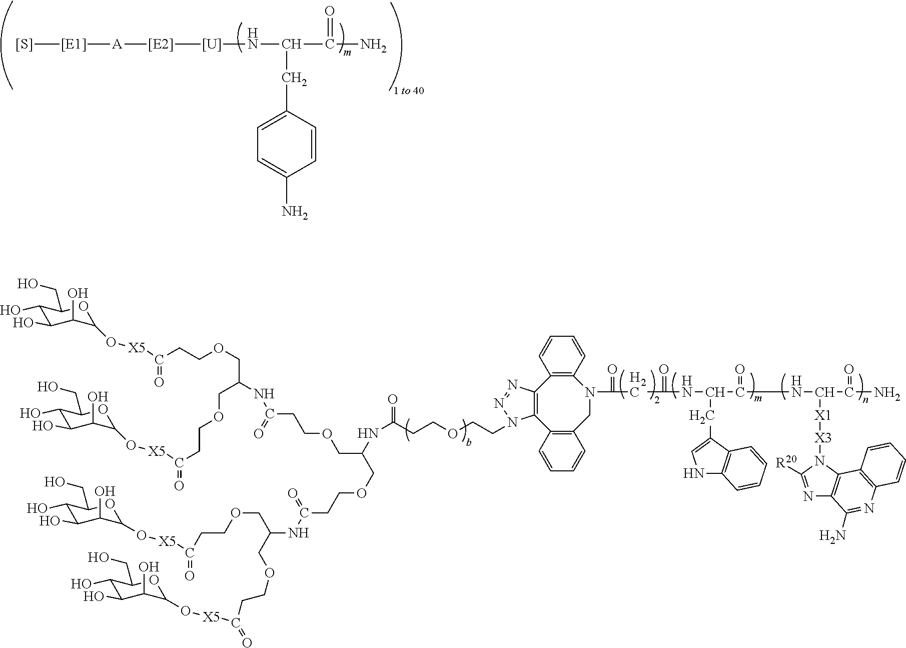

In some embodiments of cancer vaccines, the preferred composition of the first vaccine (“prime”) given to a subject is different from the preferred composition of the second vaccine (“boost”) given to a subject.

In preferred cancer treatment regimens, a subject is provided a prime immunization and at least one boost immunization, wherein each immunization is separated by an interval of between 1 and 64 days, more preferably between about 7 to 21 days. For instance, for certain cancer treatment regimens, a prime immunization is provided followed by a boost immunization at between 7 to 21 days following the prime.

In certain preferred cancer treatment regimens, a subject is provided a prime immunization that comprises nanoparticles further comprising one or more, typically between 1 and 40, peptide antigen conjugates of formula [S]-[E1]-A-[E2]-[U]-H-[D] or H-[D]-U-[E1]-A-[E2]-[S], an amphiphile of formula S-[B]-[U]-H-[D], and at least one immunostimulatory drug molecule, which is followed by a boost immunization that comprises a biological adjuvant selected from bacteria, viruses, cytokines, chemokines or the like, and nanoparticles comprising one or more, typically between 1 and 40, peptide antigen conjugates of formula [S]-[E1]-A-[E2]-[U]-H-[D] or H-[D]-U-[E1]-A-[E2]-[S], an amphiphile of formula S-[B]-[U]-H-[D] and optionally an immunostimulatory drug molecule. An unexpected finding disclosed herein is that boost immunizations comprising biological adjuvants, such as viruses, led to higher magnitude immune responses when the following criteria were met: the nanoparticles comprising one or more peptide antigen conjugates were (i) administered by the intravenous route and (ii) did not include an immunostimulant drug. Non-limiting examples of prime and boost compositions that meet these criteria are provided here below.

Non-limiting examples of the prime include:

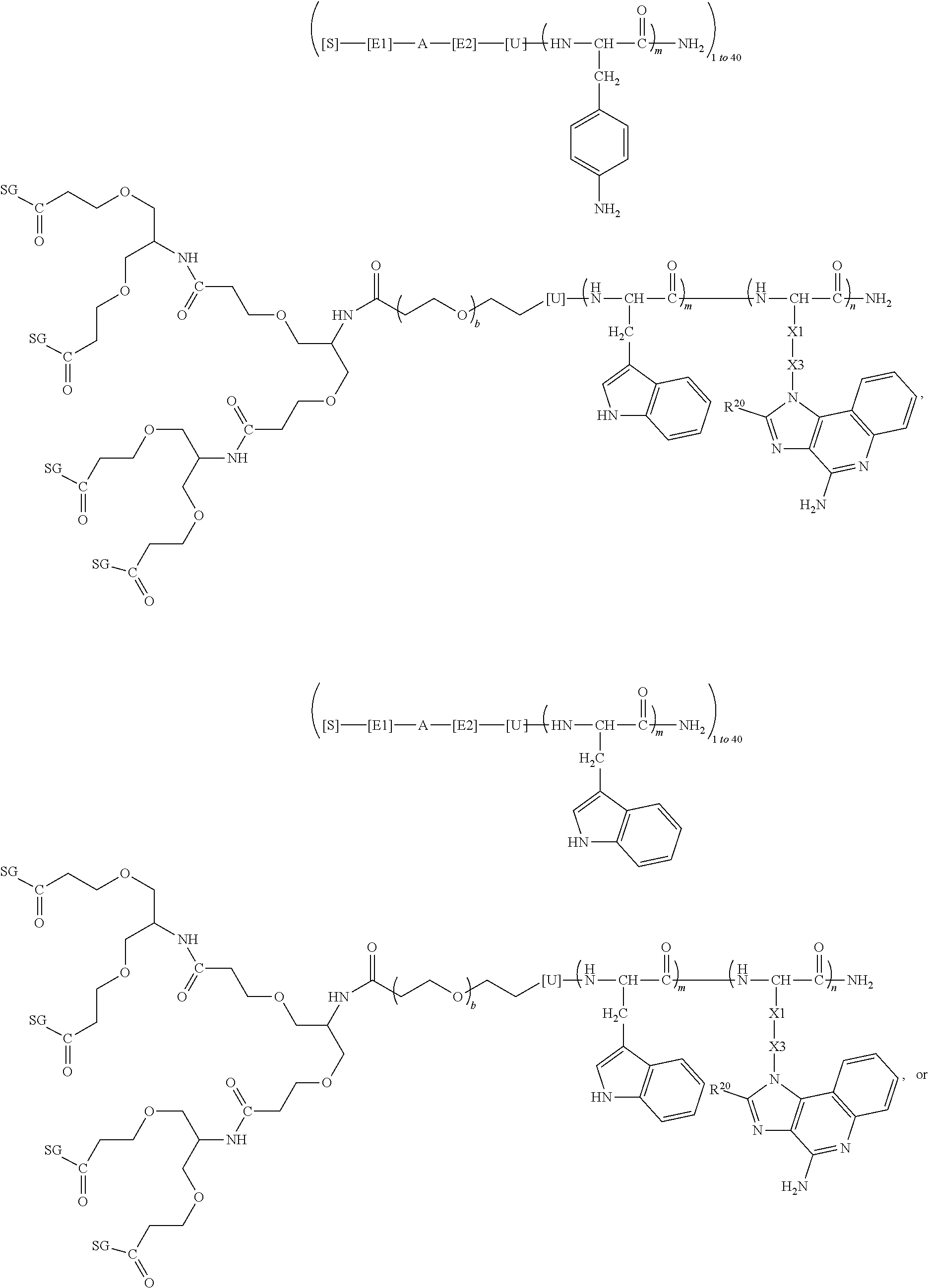

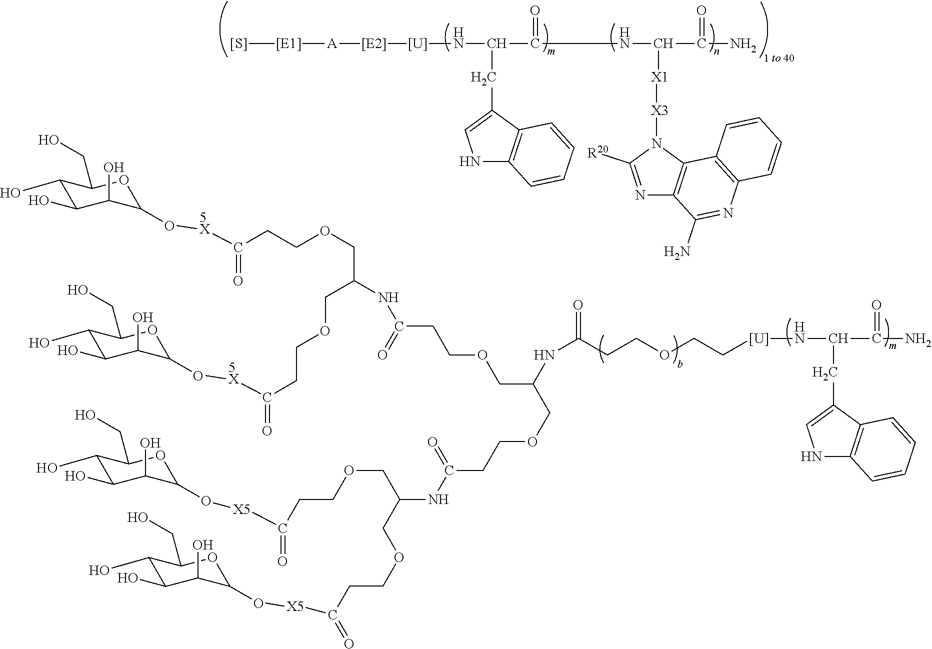

Non-limiting examples of the boost composition included for use in combination with a biological adjuvant include:

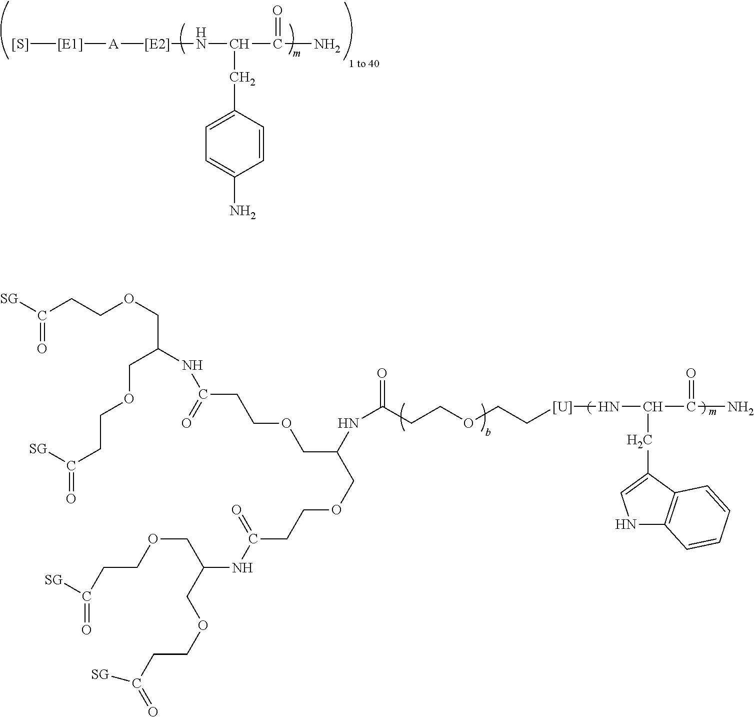

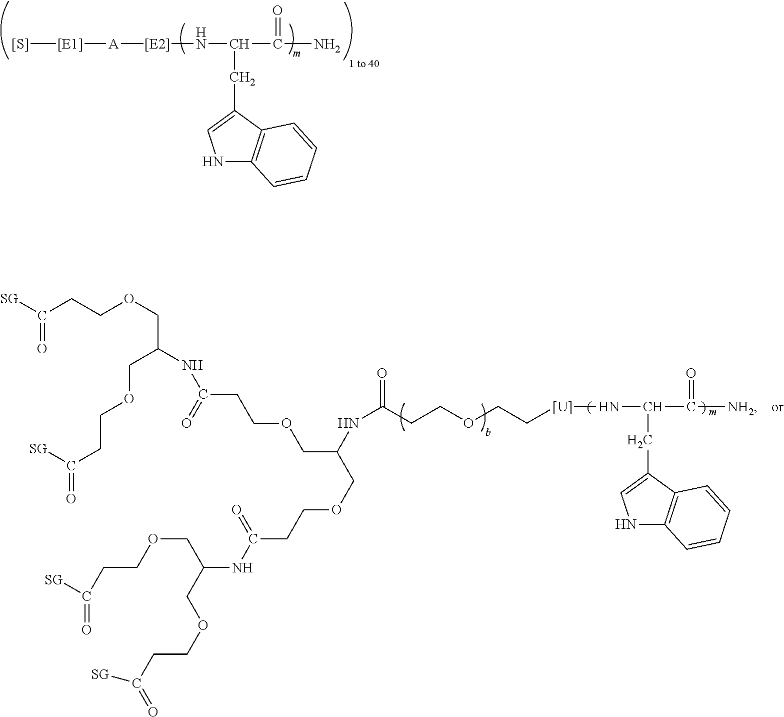

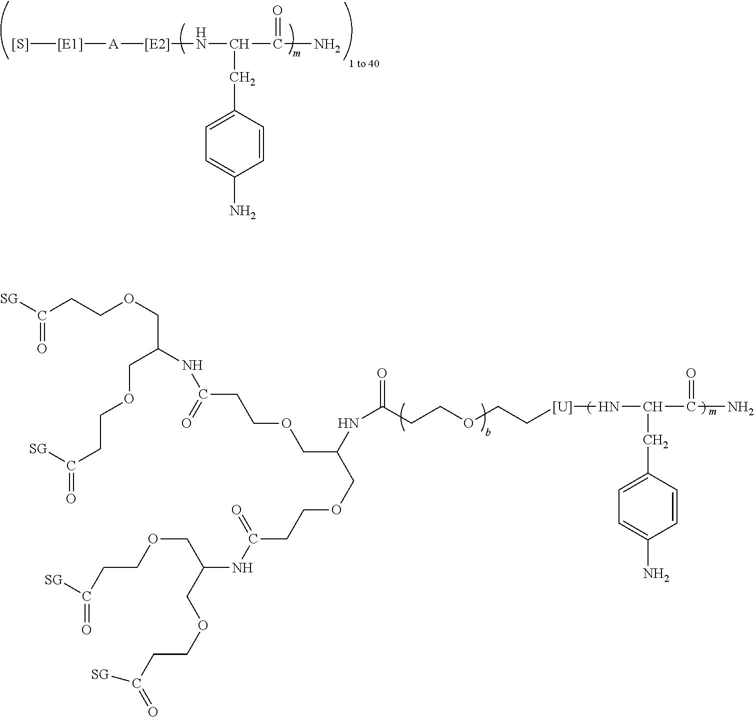

Wherein X1, X3 and X5 are each independently any suitable linker molecule; b is an integer number of monomeric units comprising the spacer and is typically between 4 and 36, such as 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35 or 36 monomeric units; SG is selected from sugar molecules, carboxylic acids, amines and/or hydroxyls that are linked to S either directly or via a suitable linker X, or, more preferably, X5; m is an integer number of repeating units of hydrophobic amino acids, which is typically selected from between 3 to 30; S is a solubilizing block, E1 is an N-terminal extension, A is an antigen selected from tumor antigens, E2 is a C-terminal extension, U is a linker and [ ] denotes that the groups are optional. In some alternative embodiments the peptide antigen conjugates have the formula H-[D]-[U]-[E1]-A-[E2]-[S].

In certain preferred embodiments of compositions of cancer vaccines comprising H further comprising tryptophan, the tryptophan is N-methylated (CAS number: 21339-55-9) such that the R group is:

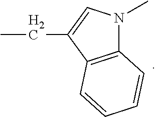

A notable finding disclosed herein was that amphiphiles with solubilizing blocks further comprising sugar molecules and/or carboxylic acids led to reduced toxicity, including hemolytic activity, when included in vaccines comprising one or more peptide antigen conjugates with average net positive charge, particularly peptide antigen conjugates with solubilizing blocks that include positively charged amino acids, such as lysine. These findings led to the design of cancer vaccines for intravenous administration that include both positively charged peptide antigen conjugates and neutral or negative amphiphile that are safer and better tolerated than cancer vaccines that lack the amphiphile. An additional notable finding was that the safety and tolerability of cancer vaccine compositions for intravenous administration could be further improved by including a drug molecule that solely blocks mTORC1 signalling. For instance, while ATP-competitive mTOR inhibitors that inhibit signalling downstream of both mTORC1 and mTORC2 were found to block the capacity of immunostimulants selected from TLR-3, TLR-7, TLR-8, TLR-7/8, TLR-9, RIGI and STING to induce CD4 T cells with Th1 phenotype, a notable finding was that rapamycin and related molecules that inhibit mTORC1, but not mTORC2, could be used to reduce the toxicity of cancer vaccines administered by the intravenous route of administration without having a deleterious impact on immunogenicity and efficacy. Therefore, in certain preferred embodiments of vaccines for preventing or treating cancer, the vaccine comprises an inhibitor of mTORC1, including but not limited to Rapamycin (Sirolimus), tacrolimus, everolimus, RAD001 (Everolimus), CCI-779 (Temsirolimus) and AP23573 (Deferolimus), and the molar ratio of total peptide antigen conjugate (i.e., the total molar amount of peptide antigen conjugate) to inhibitor of mTOR is selected from about 100:1 to about 1:4 were suitable with molar ratios of about 10:1 to 1:2 being preferred and molar ratios of about 5:1 to about 1:1 or about 4:1 to about 2:1 being most preferred.

Amphiphiles with solubilizing groups (SG) selected from sugars were found to have utility as carriers to ensure stable nanoparticle formulations as well as to reduce hemolytic activity of peptide antigen conjugates with positive net charge. In addition to these characteristics, it was found that the amphiphile could also serve a role in inducing antibody responses against tumor-associated glycans. Accordingly, it was observed that amphiphiles with SG selected from sTn, TF, sTF, Globo H, SSEA-3, GM2, GD2, GD3, Fucosyl GM1, NeuGcGM3 and poly(sialic acid) tumor associated glycans could both serve as a carrier as well as a hapten for inducing tumor glycan-specific antibodies.

Notably, it was also observed that induction of both antibodies and T cell responses against tumors led to improved efficacy as compared with inducing either antibody or T cell responses alone. Therefore, in preferred embodiments of cancer vaccines, the cancer vaccine comprises peptide antigens (A) comprising T cell epitopes and B cell epitopes, and optionally haptens comprising tumor-specific glycans. In certain preferred embodiments, peptide antigens comprising B cell epitopes are selected from glycopeptides. Various tumor associated glycopeptides are known in the art. In certain preferred embodiments of cancer vaccines, at least one peptide antigen conjugate further comprises an antigen selected from a glycopeptide selected from Mucin 1 derived peptides with O-linked glycosylation at serine and threonine residues. Non-limiting examples include the peptide antigen sequences HGVT*S*APDT*RPAPGS*T*APPA (SEQ ID NO:319), DT*RPAPGS*T*APPAHGVT*S*AP (SEQ ID NO:320), GS*T*APPAHGVT*S*APDT*RPAPGS*T*APPA (SEQ ID NO:321), GVT*S*APDT*RPAP (SEQ ID NO:322), APDT*RPAPGS*T*A (SEQ ID NO:323), GS*T*APPAHGVT*S*AP (SEQ ID NO:324), VT*S*AP (SEQ ID NO:540), DT*RPAP (SEQ ID NO:325) and GS*T*AP (SEQ ID NO:326), wherein * is an O-linked glycan and each occurrence is independently selected from sialyl lewis x, sialyl lewis a, lewis y, lewis x, Tn, sTn, TF and sTF glycans.

For certain compositions of cancer vaccines comprising peptide antigen conjugates comprising glycopeptide-based antigens and lacking a solubilizing block (i.e., [E1]-A-[E2]-[U]-H-[D], wherein A is a glycopeptide), the peptide antigen conjugate formed nanoparticle micelles of consistent size in the absence of amphiphilic carriers. A non-limiting explanation is that the glycans. present on the peptide antigen conjugate comprising a glycopeptide antigen (A). provide sufficient solubility without requiring addition of a solubilizing block on the peptide antigen conjugate or an amphiphile. Therefore, in certain preferred embodiments of cancer vaccines comprising one or more peptide antigen conjugates comprising glycopeptide-based antigens, the S block of the peptide antigen conjugate and/or amphiphile may be present or absent.

Importantly, the inventors of the present disclosure found that for cancer vaccines comprising peptide antigens further comprising B cell epitopes, e.g., peptides or glycopeptides derived from tumor cell surface proteins, rigid extensions (E1 or E2) were preferred for linking the antigen (A) to the hydrophobic block (H). In preferred embodiments, the extension used to link B cell epitopes, including peptides or glycopeptides derived from tumor cell surface proteins, is preferably selected from sequences comprising heptad repeats of formula (AA_{H}-AA_{P}-AA_{P}-AA_{H}-AA_{P}-AA_{P}-AA_{P})ₑ. In still other embodiments of cancer vaccines with T- and/or B-cell antigens, it was found that use of extensions (E1 or E2) selected from heptad repeats of formula (AA_{H}-AA_{P}-AA_{P}-AA_{H}-AA_{P}-AA_{P}-AA_{P})ₑ improved particle stability of the nanoparticle micelles that resulted in more uniform nanoparticle size. Therefore, in certain preferred embodiments of cancer vaccines, the extensions (E1 and/or E2) are selected from heptad repeats of formula (AA_{H}-AA_{P}-AA_{P}-AA_{H}-AA_{P}-AA_{P}-AA_{P})ₑ. Preferred compositions of heptad repeats of formula (AA_{H}-AA_{P}-AA_{P}-AA_{H}-AA_{P}-AA_{P}-AA_{P})ₑ for use as extensions (E1 and/or E2) are described elsewhere in the specification.

### Compositions of Vaccines for Inducing Antibody Responses

Earlier sections provided general descriptions of vaccines, including compositions of peptide antigen conjugates, amphiphiles and drug molecules (including drug molecule conjugates) that are generally preferred for use in vaccines. This section describes specific, preferred embodiments of vaccines for inducing antibody responses (“antibody vaccines”), particularly antibodies directed against minimal immunogens derived from infectious organisms, endogenously produced proteins that can be deleterious to health and haptens.

Antibody vaccines described herein comprise nanoparticles that comprise one or more peptide antigen conjugates or one or more hapten conjugates, wherein the hapten conjugate comprises a toxin or an analog or derivative of a toxin that is covalently attached to a hydrophobic block via a spacer. In preferred embodiments of antibody vaccines, the vaccine further comprises an amphiphile and an immunostimulatory drug molecule.

The one or more peptide antigen conjugates, typically between 1 and 40, each comprise an antigen (A), which is typically selected from infectious disease antigens (e.g., viral, bacterial, protozoan or fungal antigens) or endogenous proteins, such as endogenous proteins that can be deleterious to health, such as proteins implicated in cardiovascular disease, such as PCSK9 and ANGPTL3, as well as proteins involved in neurodegenerative disease, including various proteins or peptides that form amyloids, including βamyloid peptide, alpha synuclein and microtubule-associated protein Tau.

The one or more hapten conjugates each comprise a toxin or analog of a toxin, such as analogs or derivatives of drugs of abuse, nerve agents or the like. In preferred antibody vaccines, the antibody vaccine also includes one or more peptide antigen conjugates comprising antigens (A) selected from infectious disease antigens and/or non-natural CD4 helper peptides, such as PADRE. The specific antigens or haptens are selected based on the intended use of the antibody vaccine. A detailed process for selecting antigens is described in greater detail elsewhere.

In some embodiments of antibody vaccines the one or more hapten conjugates or peptide antigen conjugates (e.g., peptide antigen conjugates of formula [S]-[E1]-A-[E2]-[U]-H-[D] or H-[D]-U-[E1]-A-[E2]-[S] and/or A-[E2]-[U]-H-[D] or H-[D]-U-[E1]-A) are combined with an amphiphilic carrier (e.g., an amphiphile of formula S-[B]-[U]-H-[D]). As described earlier in the disclosure, amphiphiles of formula S-B-[U]-H-D with cone architecture comprising solubilizing blocks comprising PEG-based dendrons with between 4 to 16 solubilizing groups and PEG-based spacers with between 4 and 36 monomer units, wherein the solubilizing groups comprise sugar molecules, carboxylic acids, amines and/or hydroxyls, and the hydrophobic block comprises a poly(amino acid) of Formula I, led to improved formulation consistency and enhanced immunogenicity as compared with alternative amphiphile architectures and compositions. Though, solubilizing blocks comprising linear peptides with between 3 to 12 charged amino acids (e.g., lysine) and PEG-based spacers with between 4 and 36 monomer units, wherein the hydrophobic block comprises a poly(amino acid) of Formula I were also found to be suitable carriers for antibody vaccines.

In some embodiments of antibody vaccines, the antibody vaccine comprises one or more peptide antigen conjugates of formula S-[E1]-A-[E2]-[U]-H-[D] with net positive charge and one or more hapten conjugates and/or one or more peptide antigen conjugates of formula A-[E2]-[U]-H-[D] or H-[D]-U-[E1]-A.

In preferred embodiments of antibody vaccines, the vaccine comprises one or more, typically between 1 to 40, hapten conjugates and/or peptide antigen conjugates of formula [S]-[E1]-A-[E2]-[U]-H-[D] or H-[D]-U-[E1]-A-[E2]-[S], or, more preferably, A-[E2]-[U]-H-[D] or H-[D]-U-[E1]-A, and an amphiphile of formula S-B-[U]-H-[D] with dendron (or “cone”) architecture, wherein the amphiphile with dendron architecture further comprises a solubilizing block comprising a PEG-based dendron with between 4 to 16 solubilizing groups and a PEG-based spacer with between 4 and 36 monomer units, additionally wherein the solubilizing groups comprise sugar molecules, carboxylic acids, amines and/or hydroxyls, and the hydrophobic block comprises a poly(amino acid) of Formula I.

A non-limiting example is provided here for clarity:

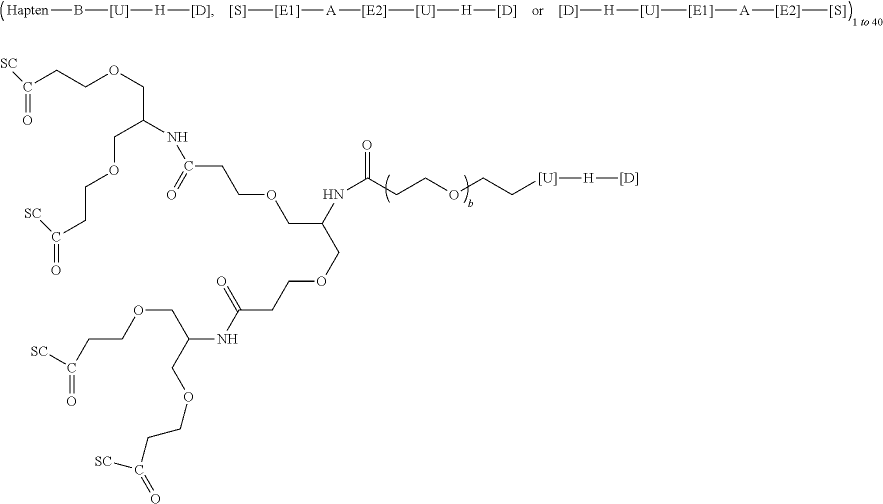

wherein b is an integer number of monomeric units comprising the spacer and is typically between 4 and 36, such as 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35 or 36 monomeric units; SG is selected from sugar molecules, carboxylic acids, amines and/or hydroxyls that are linked to S either directly or via a suitable linker X, or, more preferably, X5; the hydrophobic block (H) is typically selected from poly(amino acids) of Formula I; S is a solubilizing block; E1 is a N-terminal extension; A is an antigen that typically comprises a minimal immunogen selected from infectious disease antigens, cancer cell surface antigens, or endogenous proteins; E2 is a C-terminal extension; B is a spacer; U is a linker; D is drug molecule and [ ] denotes that the groups are optional. In some alternative embodiments the peptide antigen conjugates have the formula H-[D]-[U]-[E1]-A-[E2]-[S].

In certain other preferred embodiments of antibody vaccines, the vaccine comprises one or more, typically between 1 to 40, hapten conjugates and/or peptide antigen conjugates of formula [S]-[E1]-A-[E2]-[U]-H-[D] or H-[D]-U-[E1]-A-[E2]-[S], more preferably A-[E2]-[U]-H-[D] or H-[D]-U-[E1]-A, and an amphiphile of formula S-B-[U]-H-[D] with linear architecture, wherein the amphiphile with linear architecture further comprises a solubilizing block comprising a peptide with between 3 to 12 charged amino acids and a PEG-based spacer with between 4 and 36 monomer units, and the hydrophobic block comprises a poly(amino acid) of Formula I.

In preferred embodiments of antibody vaccines, the vaccine further comprises a drug molecule selected from immunostimulants and is typically selected from agonists of TLR-1, TLR-2, TLR-6, TLR-7, TLR-8, TLR-7/8a or TLR-9. In preferred embodiments, the immunostimulant is selected from an imidazoquinoline of Formula IV, which is linked to the peptide antigen conjugate (or hapten conjugate) and/or amphiphile via a covalent bond. A non-limiting example is provided here for clarity:

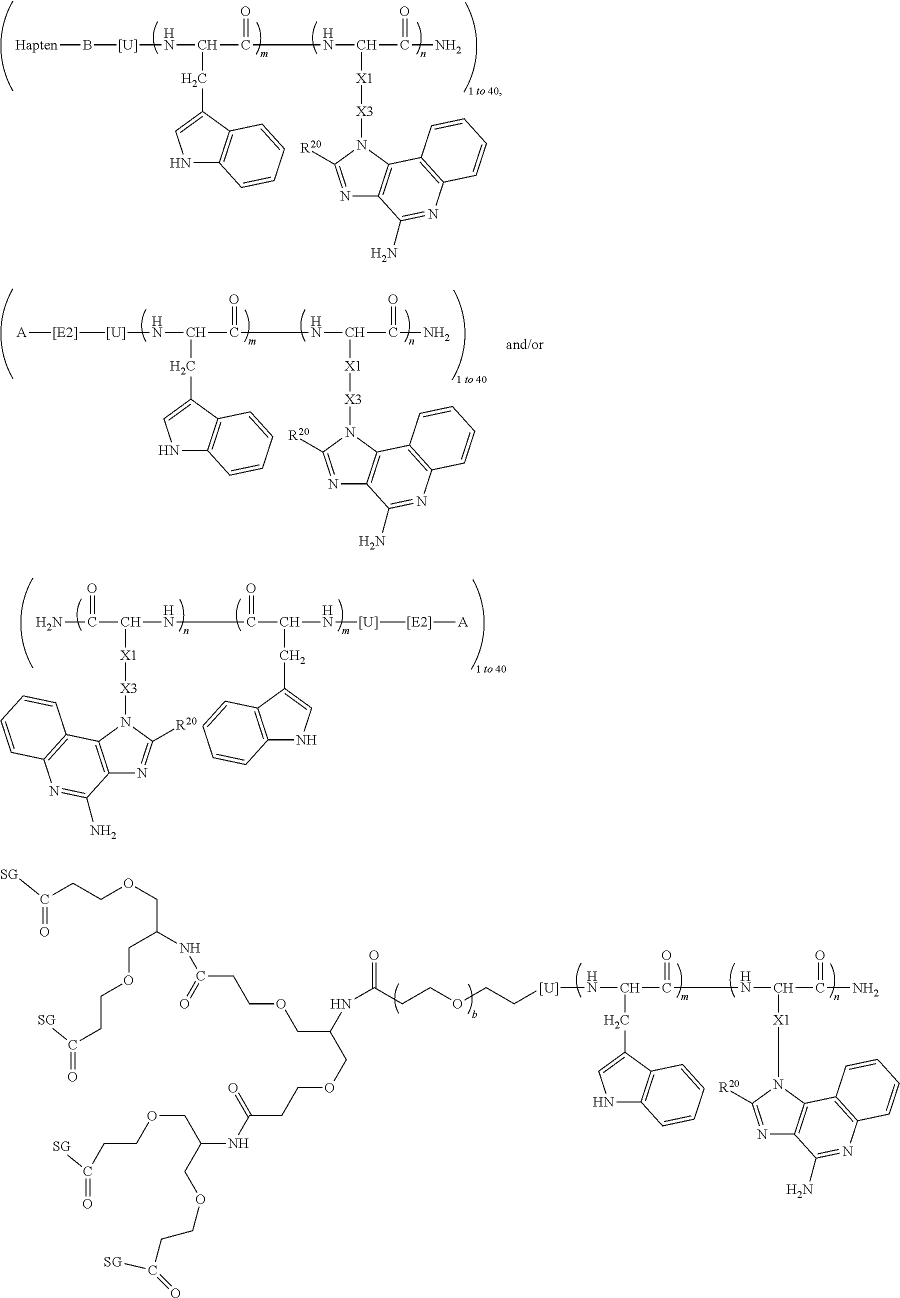

An unexpected finding disclosed herein is that the use of PEG- and/or peptide-based extensions (E1 or E2) in peptide antigen conjugates led to improved manufacturability as well as enhanced immunogenicity of mosaic nanoparticle vaccines comprising one or more peptide antigen conjugates and an amphiphilic carrier (‘amphiphile’). Accordingly, the inventors of the present disclosure found that antibody vaccines comprising nanoparticles further comprising peptide antigens linked to hydrophobic blocks via E1 or E2 comprising PEG and/or peptides with between 1 to 36 monomeric units and amphiphiles with spacers (B) comprising PEG with a number of monomeric units less than or equal to the sum of the monomeric units of the antigen and E1 or E2 extension led to stable nanoparticle micelles that induced higher magnitude antibody titers than when the PEG-based spacer (B) of the amphiphile had a greater number of monomeric units than the sum of the number of antigen and E1 (or E2) monomeric units of the peptide antigen conjugate. Additionally, the inventors found that the N-terminal position was preferred for incorporating the PEG-based extension. Thus, in preferred embodiments of antibody-based vaccines, the hapten or peptide antigen is linked to the hydrophobic block through a PEG- and/or peptide-based E1 or E2 extension, though, more preferably through an E1 extension. A non-limiting example is provided here for clarity:

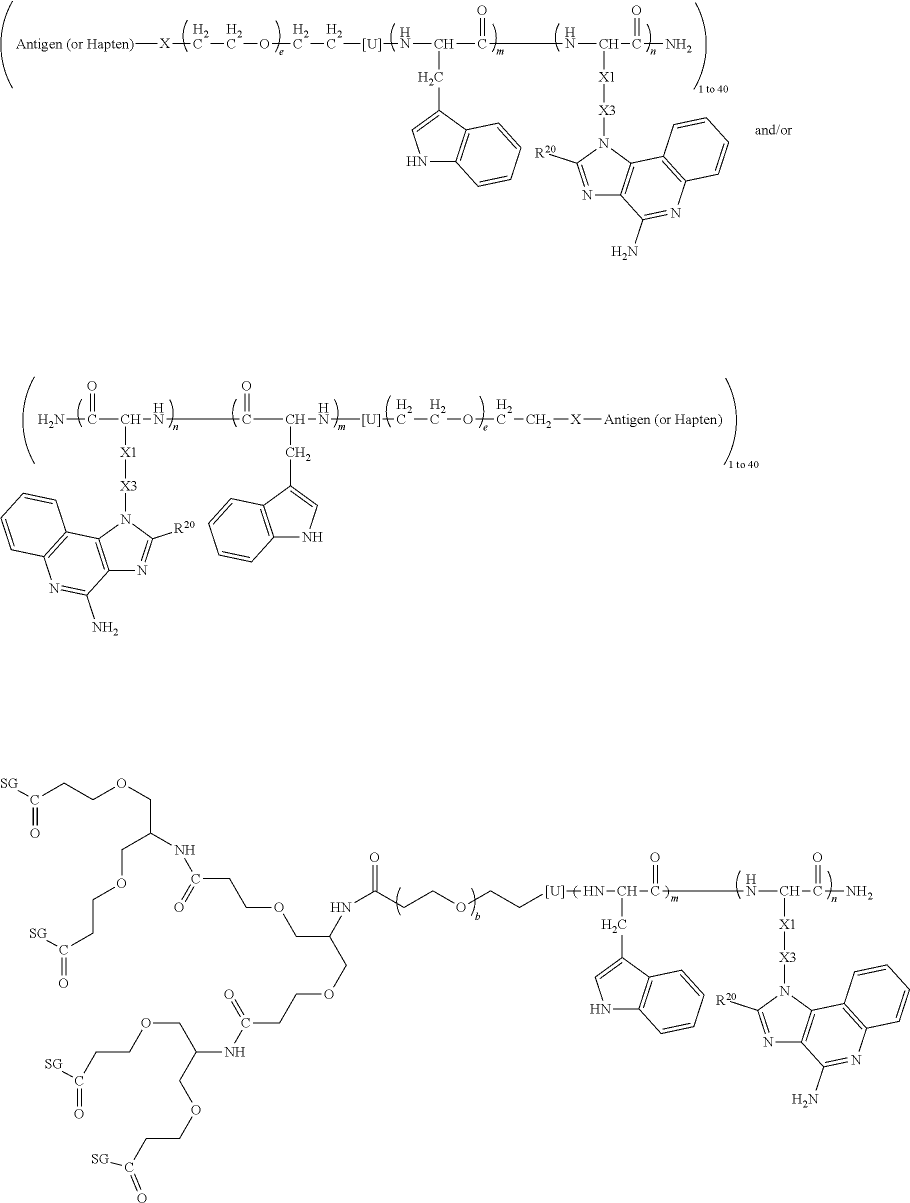

wherein X, X1 and X3 are each independently any suitable linker molecule, b is an integer number of monomeric units comprising the spacer and is typically between 4 and 36, such as 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35 or 36 monomeric units; e is an integer number of monomeric units comprising the extension (E1 or E2) and is typically selected from between 1 to 36, though, more preferably, between 4 and 24, such as 4, 5, 6, 7, 8, 9, 10 11, 12, 13, 14, 15, 16, 17, 18, 19, 20, 21, 22, 23 or 24 monomeric units; SG is selected from sugar molecules, carboxylic acids, amines and/or hydroxyls that are linked to S either directly or via a suitable linker X, or, more preferably, X5; A is an antigen that typically comprises a minimal immunogen selected from infectious disease antigens or endogenous proteins, U is a linker and [ ] denotes that the groups are optional. In preferred embodiments, e is selected from between 4 and 24 monomer units and b is selected from between 4 and 24 monomer units.

In certain preferred embodiments, the solubilizing groups are selected from mannose and the above structure becomes:

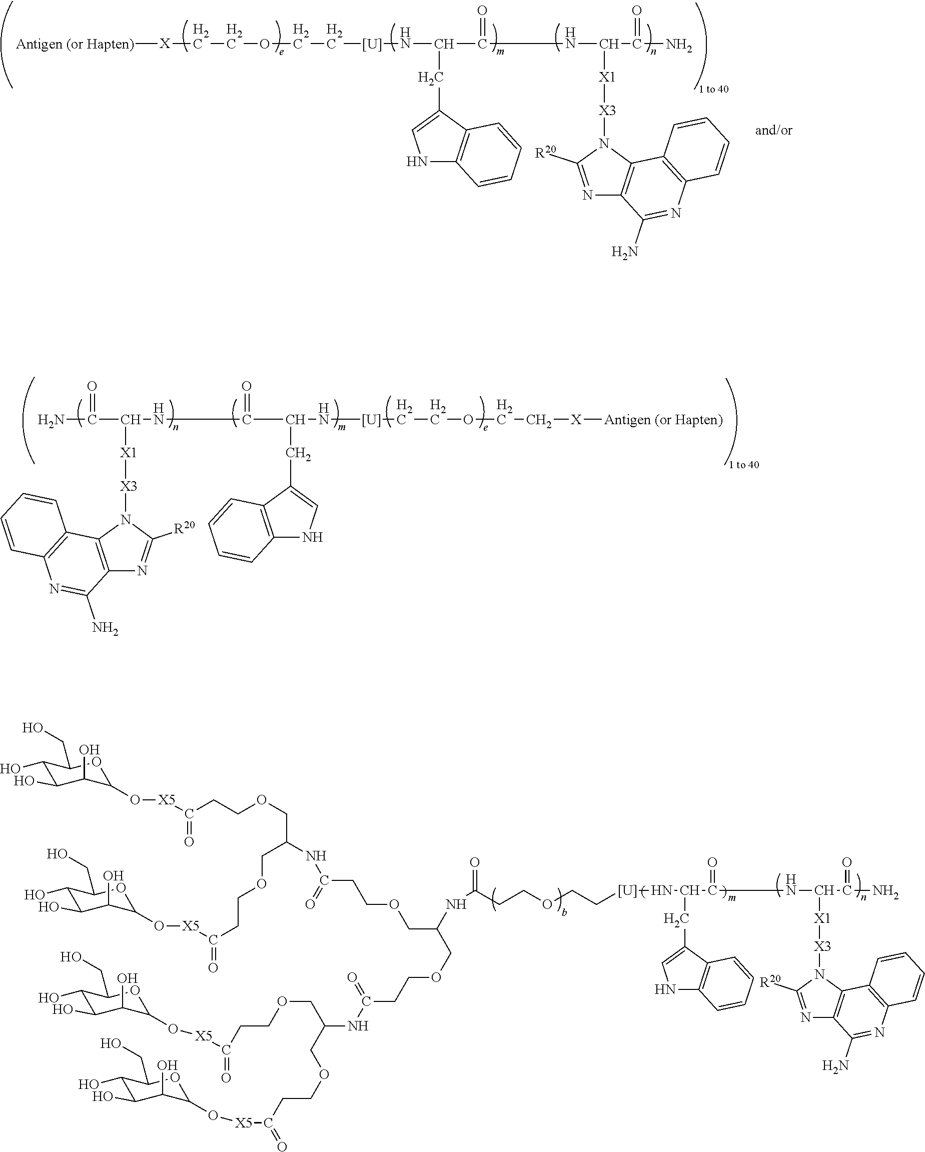

wherein X5 is any suitable linker molecule.

While introduction of a PEG and/or peptide-based extension between the antigen (A) (or hapten) and hydrophobic block (H) was found to generally improve solubility and increase the magnitude of antibody responses directed to the antigen (or hapten), an additional notable finding was that increasing the rigidity of the extension (E1 or E2) generally further enhanced antibody responses. The inventors of the present disclosure found that suitable peptides comprising the extension (E1 or E2) between the peptide antigen (A) and hydrophobic block (H), include but are not limited to peptides of between about 4 to 24 amino acids in length comprising amino acids selected from glycine, serine, threonine, alanine, proline and ethylene oxide, or, more preferably, peptides comprising heptad repeats of formula (AA_{H}-AA_{P}-AA_{P}-AA_{H}-AA_{P}-AA_{P}-AA_{P})ₑ. Preferred compositions of heptad repeats of formula (AA_{H}-AA_{P}-AA_{P}-AA_{H}-AA_{P}-AA_{P}-AA_{P})ₑ for use as extensions (E1 and/or E2) are described elsewhere. In some embodiments, the extension comprises a combination of PEG-based linkers, peptide sequences, more preferably heptad repeats, and/or aliphatic linkers. In preferred embodiments of vaccines for inducing antibody responses, e.g., minimal immunogen vaccines, the antigen (A) is linked to the hydrophobic block (H) via an extension selected from a heptad repeat of formula (AA_{H}-AA_{P}-AA_{P}-AA_{H}-AA_{P}-AA_{P}-AA_{P})ₑ as shown in the non-limiting example below:

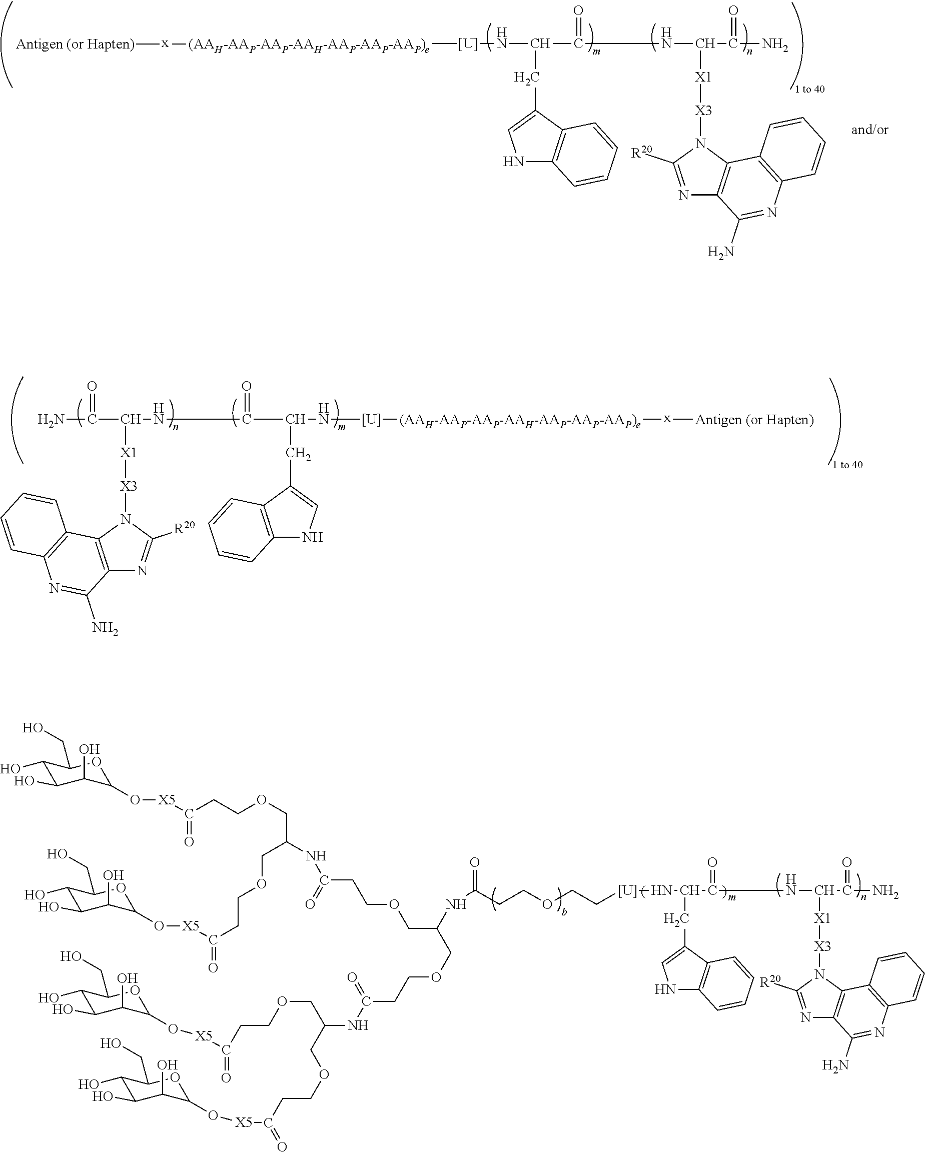

In the above example, wherein the imidazoquinoline drug molecule is not present on the amphiphile the structure is:

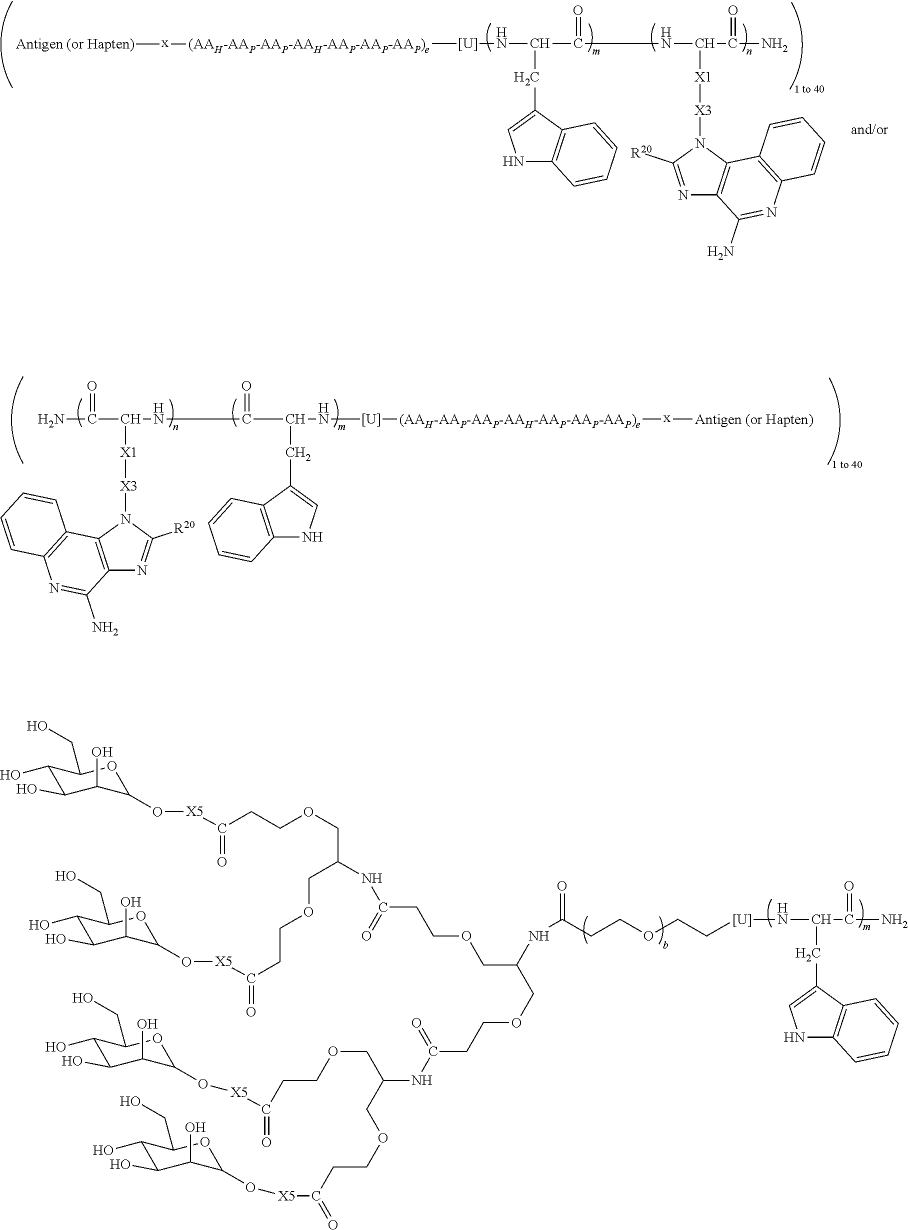

In certain preferred embodiments of vaccines for inducing antibodies comprising H further comprising tryptophan, the tryptophan is N-methylated (CAS number: 21339-55-9), such that the R group is:

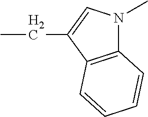

### Compositions of Vaccines for Treating Cardiovascular and Neurodegenerative Diseases

In preferred embodiments of a vaccine for treating cardiovascular or neurodegenerative disease, the vaccine comprises an amphiphile of formula S-B-[U]-H-[D] and one or more peptide antigen conjugates of formula A-[E2]-[U]-H-[D] or H-[D]-[U]-[E1]-A, wherein the one or more peptide antigen conjugates are selected from antigens derived from endogenous proteins implicated in cardiovascular or neurodegenerative disease.

A non-limiting example of a vaccine for treating cardiovascular or neurodegenerative disease is provided here for clarity, wherein the vaccine comprises one or more peptide antigen conjugates of formula:

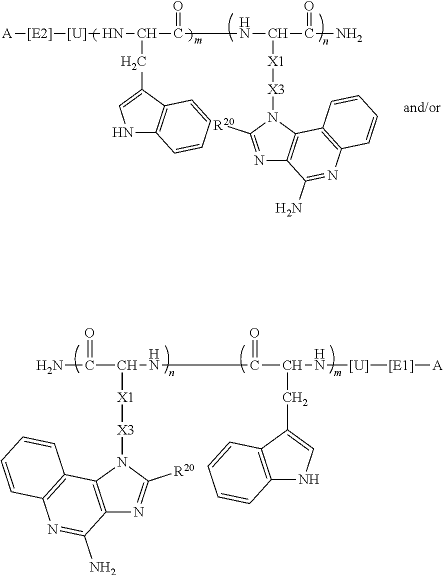

and an amphiphilic carrier of formula:

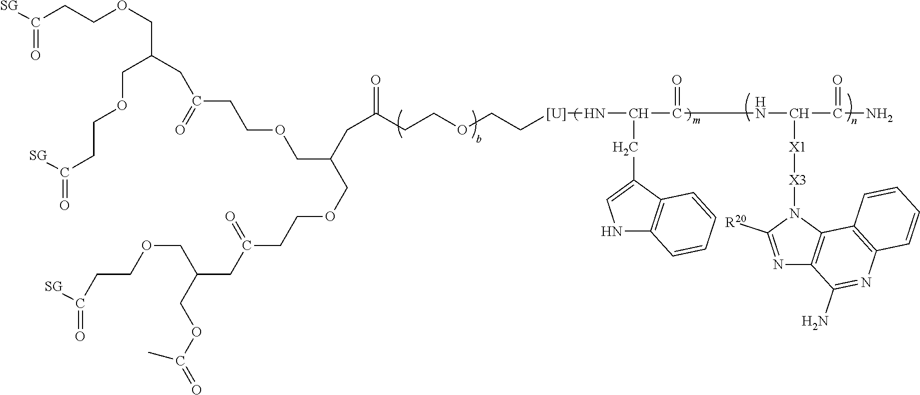

wherein X1 and X3 are each independently any suitable linker molecule, b is an integer number of monomeric units comprising the spacer and is typically between 4 and 36, such as 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35 or 36 monomeric units, though, preferably between about 4 and 24 monomer units in length; SG is selected from sugar molecules, carboxylic acids, amines and/or hydroxyls that are linked to S either directly or via a suitable linker X, or, more preferably, X5; U is a linker typically comprising a triazole (if present); E1 (or E2) are typically present and selected from PEG or peptides of between 1 and 36 monomer units in length, though typically, between about 4 to 24 monomer units in length; A is a peptide antigen selected from peptide sequences derived from endogenous proteins involved in cardiovascular disease, such as PCSK9 and/or ANGPTL3, including but not limited to RGYLTKILHVFHGLLPGFLVKMSGDLLE (SEQ ID NO:106), PGFLVKMSGDLLE (SEQ ID NO: 107), PGFLVKnSGDLLE (SEQ ID NO:108) (wherein n=norleucine), PGFLVKMSSDLLG (SEQ ID NO:109), PGFLVKnSSDLLG (SEQ ID NO:110) (wherein=norleucine), SIPWNLERITPPR (SEQ ID NO:111), SIPWNLERITPPR (SEQ ID NO:112), SIPWNLE (SEQ ID NO:113), SIPWNLEKVTPPR (SEQ ID NO:114), SIPWNLDRVTPPR (SEQ ID NO:115), NVPEEDGTRFHRQASKC (SEQ ID NO:116), NVPEEDGTRFHRQASK (SEQ ID NO:117), PEEDGTR (SEQ ID NO:118), NVPEEDG (SEQ ID NO:119), NVPEEDATRFHRQGSK (SEQ ID NO: 120), LFAPGEDIIGASSDCSTCFVSQSGTSQAAA (SEQ ID NO: 121), CSTCFVSQSGTSQAAA (SEQ ID NO: 122), STCFVSQSGTSQAAA (SEQ ID NO:123), STBFVSQSGTSQAAA (SEQ ID NO:124), STBFVSQ (SEQ ID NO: 125), MFTIKLLLFIVPLVISSRIDQDNSSFDSLSPEPKSRFAMLDDVKILANGLLQLGHGLKDFVHK TKGQIND (SEQ ID NO:126), EPKSRFAMLDDVKILANGLLQLGHGLKDFVHKTKGQIND (SEQ ID NO:127), EPKSRFAMLDDVKI, (SEQ ID NO:128), MLDDVKILANGLLQ (SEQ ID NO: 129), LANGLLQLGHGLKD (SEQ ID NO:130), LGHGLKDFVHKTKG (SEQ ID NO:131), LKDFVHKTKGQIND (SEQ ID NO:132), RFAMLDDVKILANGLLQLGH (SEQ ID NO: 133), GLLQLGHGLKDFVHKTKGQI (SEQ ID NO:134), IFQKLNIFDQSFYDLSLQTSEIKEEEKELRRTTYKLQVKNEEVKNMSLELNSKLESLLEEKILL QQKVKY (SEQ ID NO: 135), or peptide sequences derived from endogenous proteins involved in neurodegenerative disease, such as beta peptide, alpha synuclein, and microtubule-associated protein Tau or any fragments or derivatives thereof; and [ ] denotes that the groups are optional.

A non-limiting example of a cardiovascular vaccine is provided herein for clarity:

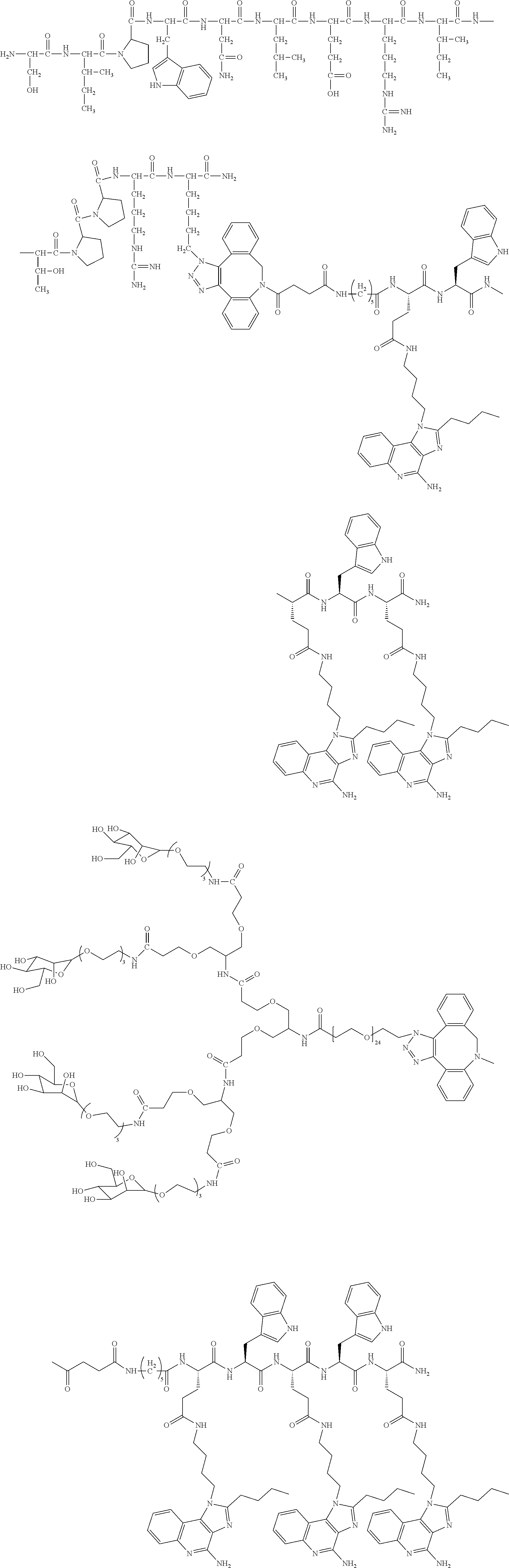

wherein, in the above example, the peptide antigen conjugate comprises a peptide antigen (sequence: SIPWNLEKVTPPR (SEQ ID NO: 114) linked to a poly(amino acid)-based hydrophobic block (H) of Formula I linked imidazoquinolines of Formula IV through a triazole linker.

In certain preferred embodiments of cardiovascular vaccines, the one or more peptide antigen conjugates typically comprise an extension (E1 or E2) selected from PEG, e.g., (CH₂—CH₂—O)ₑ, where e is an integer number of units of ethylene oxide, typically selected from between 1 and 36, more preferably between 4 and 24 monomeric units; peptides, such as (Gly-Ser)₂₋₁₂, (Gly-Gly-Gly-Gly-Ser)₁₋₄, (Ala-Pro)₂₋₁₂, (Ile-Ala-Ala-Ile-Glu-Ser-Lys)₁₋₄, (Ile-Ala-Ala-Ile-Lys-Ser-Lys)₁₄, or (Ile-Ala-Ala-Ile-Glu-Ser-Glu)₁₋₄; or, combinations of PEG- and peptide-based linkers.

### Compositions of Vaccines for Treating or Preventing Infectious Diseases

In preferred embodiments of a vaccine for preventing or treating an infectious disease, the vaccine comprises an amphiphile of formula S-B-[U]-H-[D] and one or more peptide antigen conjugates of formula [S]-[E1]-A-[E2]-[U]-H-[D] and/or H-[D]-[U]-[E1]-A-[E2-[S]. Wherein the antigen selected for preventing or treating an infectious disease is a B cell epitope, the peptide antigen conjugate does not have a solubilizing block and the peptide antigen conjugate has the formula A-[E2]-[U]-H-[D] or H-[D]-[U]-[E1]-A.

A non-limiting example of a vaccine for preventing or treating infectious diseases that comprises peptide antigen conjugates further comprising one or more antigens (A) that comprise a B cell epitope is provided here for clarity, wherein the vaccine comprises one or more peptide antigen conjugates of formula:

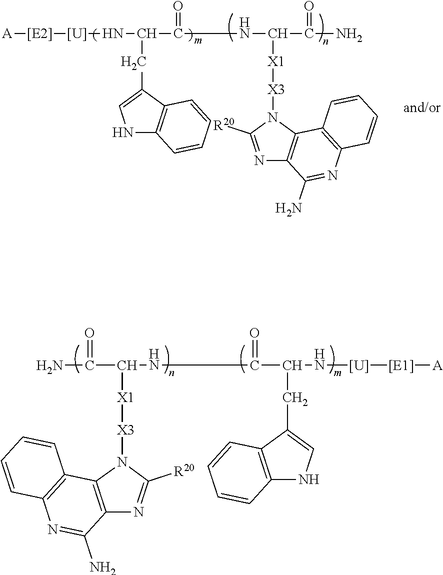

and an amphiphilic carrier of formula:

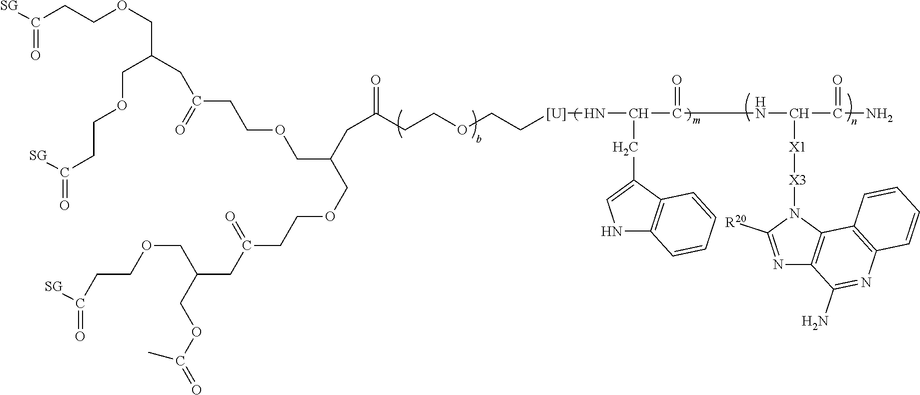

wherein X1 and X3 are each independently any suitable linker molecule, b is an integer number of monomeric units comprising the spacer and is typically between 4 and 36, such as 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35 or 36 monomeric units; SG is selected from sugar molecules, preferably mannose or Sialyl Lewis x sugars, carboxylic acids, amines and/or hydroxyls that are linked to S either directly or via a suitable linker X, or, more preferably, X5; U is a linker preferably selected from triazole (if present); E1 (or E2) are typically present and selected from PEG or peptides of between 1 and 36 monomer units in length, though typically, between about 4 to 24 monomer units in length; A is a peptide antigen selected from peptide sequences derived from infectious organisms; and [ ] denotes that the groups are optional.
General Features of Vaccines that are Useful for Promoting Tolerance

The inventors of the present disclosure identified compositions that provided unexpected improvements in manufacturability, safety and/or efficacy of vaccines for inducing tolerance, including vaccines for the treatment of allergies and autoimmunity.

In preferred embodiments of vaccines for inducing tolerance, the vaccine comprises one or more peptide antigen conjugates of formula [S]-[E]-A-[E2]-[U]-H, an amphiphile of formula S-[B]-[U]-H and optionally one or more distinct immunomodulatory drug molecules, which may be either linked to the hydrophobic block of the peptide antigen conjugate (e.g., [S]-[E]-A-[E2]-[U]-H-D), amphiphile (e.g., S-[B]-[U]-H) or both, or provided as a drug molecule conjugate (e.g., D-[B]-[U]-H), or free drug, D.

The one or more peptide antigen conjugates each comprise an antigen (A) selected from autoantigens or allergens that preferably comprise one or more T cell epitopes. In preferred embodiments, vaccines for inducing tolerance typically comprise more than one composition of peptide antigen conjugate, preferably between 1 and 40 unique peptide antigen conjugates, each with a distinct peptide antigen (A) composition. The process for selecting antigens (A) is described in detail elsewhere.

While various options for solubilizing blocks (S) exist and are described in greater detail elsewhere in the specification, the authors of the present disclosure identified specific architectures and compositions of solubilizing blocks (S) that led to improved manufacturing as well as enhanced safety and efficacy of vaccines for inducing tolerance. Accordingly, solubilizing blocks (S) with dendron architecture having between 2 and 32 solubilizing groups, preferably between about 4 and 8 solubilizing groups, were found to be optimal for generally improving manufacturing and peptide antigen conjugate loading into vaccines as compared with solubilizing blocks with linear or brush architectures, which tended to require higher net surface charge for particle stabilization. Additionally, the specific solubilizing group (SG) composition was found to have a substantial impact on the efficacy of vaccines for inducing tolerance. Indeed, an unexpected finding by the inventors of the present disclosure was that vaccines for inducing tolerance comprising amphiphiles of formula S-[B]-[U]-H optionally comprising a drug molecule (e.g., S-[B]-[U]-H-D) with solubilizing blocks (S) comprising dendrons comprising negatively charged solubilizing groups and/or saccharides and having net negative or near neutral charge led to enhanced efficacy for treating autoimmune diseases.

Therefore, in preferred embodiments, the solubilizing block of amphiphiles used in vaccines for inducing tolerance typically comprises dendron architecture with solubilizing groups (SG) selected from carboxylic acid, phosphoserine (or glycerophosphoserine), glucose, mannose, glucosamine, n-acetylglucosamine, galactose, galactosamine, n-acetyl-galactosamine and/or agonists of CD22a, which may be linked either directly or indirectly via a linker to the terminal functional groups (FGt) of the solubilizing block (S) with dendron architecture through any suitable means, though, in preferred embodiments the solubilizing group is linked to FGt via an amide bond. In certain preferred embodiments, solubilizing groups (SG) comprising sugar molecules are linked to the solubilizing block (S) via an alpha- or/beta-linkage at the anomeric carbon. In still other embodiments, the solubilizing group (SG) is the terminal functional group of the dendron, as may be the case, e.g., for FGt comprising a carboxylic acid.

A non-limiting example of a solubilizing block (S) comprising solubilizing groups (SG) further comprising carboxylic acids, wherein the solubilizing block (S) is linked either directly or indirectly via a spacer (B) and/or Linker U to a hydrophobic block (H) is shown here for clarity:

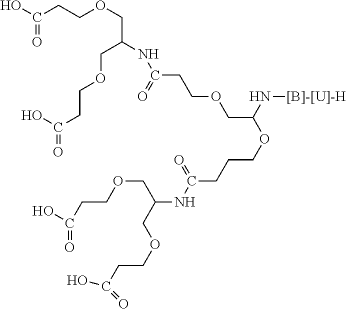

An unexpected finding was that amphiphiles of the above structure were pH-responsive at pH near physiologic pH 7.4 leading to reduced solubility and aggregation when dispersed in solution at or near pH 7.4, e.g., between pH 7.0 and 7.3. However, subtle changes to the chemical composition of solubilizing blocks with dendron architecture having carboxylic acids were found to affect the range over which the resulting amphiphiles were pH-responsive. In non-limiting examples, substitution of the terminal functional groups FGt with beta-alanine yielded amphiphiles of the following structure that were not found to exhibit pH responsive properties down to at least pH 6.0:

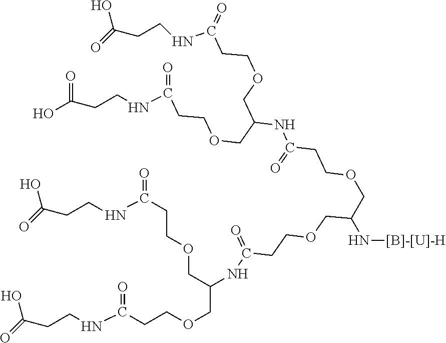

Similarly, amphiphiles comprising lysine-based dendrons wherein the primary amines of the lysine, i.e., FGt, were substituted with succinic acid led to amphiphiles that formed nanoparticles that were stable near physiologic pH 7.4. A non-limiting example of an amphiphile comprising lysine-based dendrons wherein the primary amines of the lysine, i.e., FGt, were substituted with succinic acid is shown here for clarity:

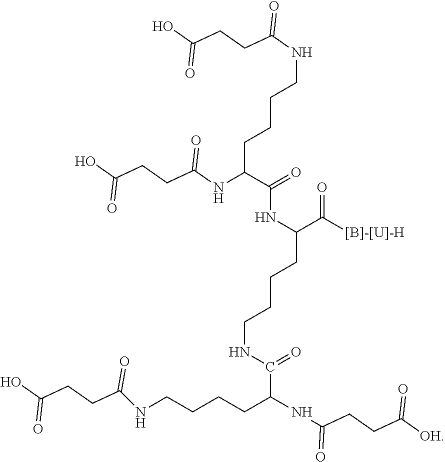

Based on these findings, preferred embodiments of vaccines, including vaccine for inducing tolerance, that comprise amphiphiles with negative charge comprise dendrons with terminal functional groups substituted with beta-alanine and/or succinic acid.

An additional non-limiting example of a solubilizing block (S) comprising solubilizing groups (SG) further comprising a saccharide, wherein the solubilizing block (S) is linked either directly or indirectly via a spacer (B) and/or Linker U to a hydrophobic block (H) is shown here for clarity:

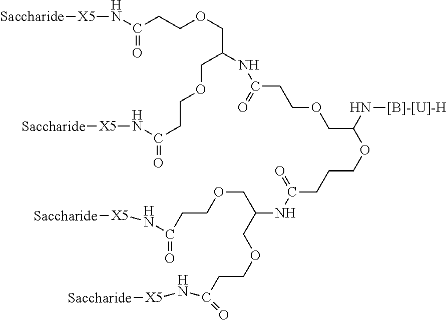

wherein X5 is any suitable linker, typically selected from lower alkyl and/or ethylene oxide and the saccharide is typically selected from

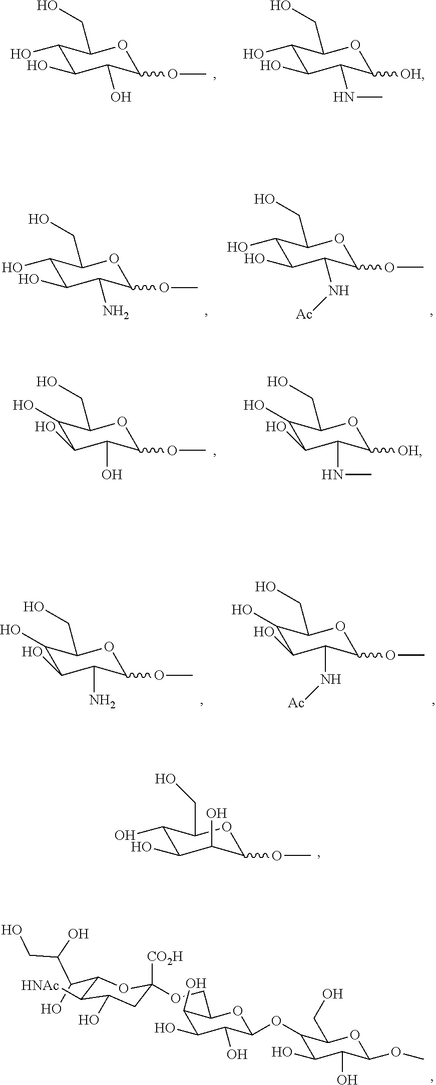

or a combinations thereof.

Both the peptide antigen conjugate and amphiphile comprise a hydrophobic block (H). Various options for hydrophobic blocks (H) exist and are described in greater detail elsewhere in the specification; however, the authors of the present disclosure identified hydrophobic blocks (H) that have utility for use with tolerance vaccines. In preferred embodiments, the hydrophobic block (H) comprises a poly(amino acid) of Formula I comprising monomer units selected from hydrophobic amino acids (M) further comprising aryl or heteroaryl groups and/or reactive amino acids (N) linked to hydrophobic drug molecules (D). In some embodiments of vaccines for inducing tolerance, the hydrophobic block (H) of amphiphiles and/or peptide antigen conjugates comprises a poly(amino acid) of Formula I, wherein the poly(amino acid) is comprised entirely of hydrophobic amino acids (M). Non-limiting examples are provided here for clarity:

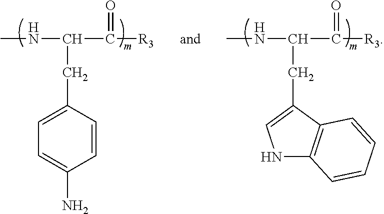

Additional non-limiting examples include poly(amino acids) of Formula I comprising hydrophobic amino acids M that are AHR agonists, including:

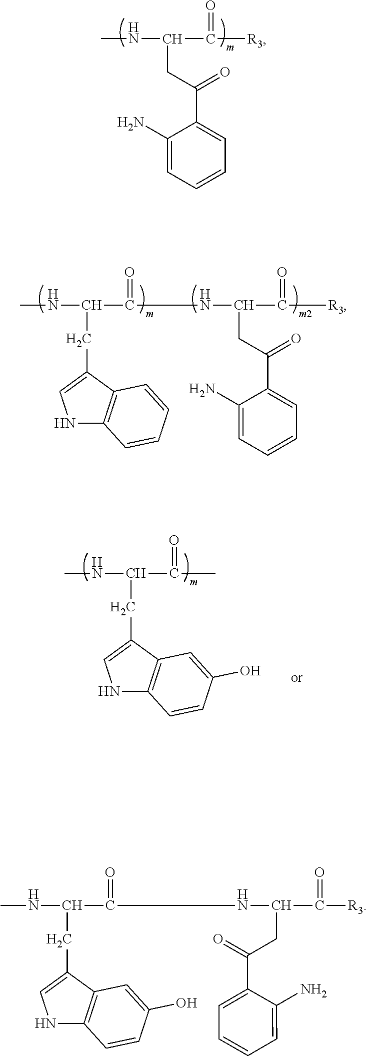

Drug molecules (D) with immunomodulatory properties, referred to as immunomodulatory drugs, may be added to or co-administered with vaccines for inducing tolerance to further improve efficacy. Suitable drug molecules include immunomodulators that can promote regulatory T cell priming, trans-differentiation, expansion or stabilization (“Treg promoting immunomodulators”), which includes phosphoinositide-3-kinase (PI3K), AKT and mammalian target of rapamycin (mTOR) inhibitors, such rapamycin, everolimus, torin 1, torin 2, INK-128, dactolisib, AZD8055, KU-00639874 and any analogs, derivatives or salt forms thereof; cyclin dependent kinase (CDK8) and/or CDK19 inhibitors, such as Cortistatin, CCT251545, CCT251921, Senexin A and BRD6968; retinoic acid-related orphan gamma t (RORγt) inhibitors, such as SR1555 or SR1001; certain histone deacetylases (HDACs), such as trichostatin-A (TsA), suberoylanilide hydroxamic acid (SAHA, or “vorinostat”), or butyrate, or, more preferably inhibitors of HDAC9, such as TMP269; agonists of aryl hydrocarbon receptor (AHR), such as indole, indolo[3,2-b]carbazole (ICZ), kynurenine, kynurenic acid, 5-hydroxy tryptophan, tryptamine, indol-3-acetic acid and ITE (cas: 448906-42-1) (see: Gutierrez-Vazquez, C., et al. Immunity Review, 2018); substrates for indoleamine 2,3-dioxygenase (IDO); agonists of retinoic acid receptors (RAR), such as all-trans retinoic acid, TTNPB (cas: 71441-28-6), AM580, BMS753, BMS961 and the like; certain adenosine receptor agonists, e.g., agonists of A_{2A}, such as ATL-146e, YT-146, (N6-(2-(3,5-dimethoxyphenyl)-2-(2-methylphenyl)ethyl)adenosine (DPMA), regadenoson, UK-432,097, zeatin; and, agonists of TGF-β, IL-17, IL-2 and IL-10 receptors, including naturally occurring proteins and/or antibodies. Note: “Treg promoting immunomodulators” may also be described more generally as immunosuppressants.

An unexpected finding by the inventors of the present disclosure is that compositions of vaccines for inducing tolerance that include certain compositions of immunostimulants and one or more Treg promoting immunomodulators led to significantly higher magnitude of T cell induction as compared with vaccine compositions without the immunostimulants. This was unexpected because immunostimulants are believed to oppose regulatory T cell induction and immune suppression.

Non-limiting examples of immunostimulants that were found to be effective for use in combination with Treg promoting immunomodulators include but are not limited to: agonists of C-type lectin receptors (CLR), such as trehalose-6,6-dibenhenate, agonists of nucleotide-binding oligomerization domain (NOD)-like, such as muramyl dipeptide; agonists of TLR-7, such as imidazoquinolines; agonists of TLR-4 such as lipopolysaccharide or derivatives thereof, such as monophosphoryl lipid A (MPL-A); and agonists of STING, such as CDNs (e.g., c-di-AMP) and diABZI.

The preferred means of incorporating drug molecules into vaccines for inducing tolerance depends, in part, on the composition of the drug molecule.

Immunostimulants and/or Treg promoting immunomodulators that are poorly water soluble, i.e., hydrophobic drug molecules, may be admixed with amphiphiles and/or peptide antigen conjugates (e.g., D+S-[B]-[U]-H and/or [S]-[E1]-A-[E2]-[U]-H) and incorporated into the hydrophobic core of particles comprising the amphiphiles and/or peptide antigen conjugates through non-covalent interactions. Non-limiting examples include immunostimulants comprising fatty acids, such as lipopeptide-based agonists of TLR-1, -2 and/or -6 as well as lipid-based agonists of TLR-4 and CLRs (e.g., mincle); immunostimulants comprising polycyclic heteroaryls, such as imidazoquinoline based agonists of TLR-7 and -8, as well as diABZI-based agonists of STING, and any derivatives thereof, such imidazoquinoline or pip-diABZI molecules linked to fatty acid, cholesterol or other hydrophobic moieties through the N1 of imidazoquinoline or nitrogen of piperazine, respectively; macrolide-based inhibitors of mTOR, such as rapamycin, and any of the various heterocyclic aromatic inhibitors of mTOR/PI3K/AKT (e.g., KU-0062794, Torin 1, Torin 2, etc.), CDK8/19 (e.g., Cortistatin), retinoic acid-related orphan gamma t (RORγt) inhibitors, such as SR1555; certain histone deacetylases (HDACs), such as TMP269; certain agonists of aryl hydrocarbon receptors (AHR), such as indole, indolo[3,2-b]carbazole (ICZ), 3,3 diindolomethane and ITE; agonists of retinoic acid receptors (RAR), such as all-trans retinoic acid, TTNPB (cas: 71441-28-6), AM580, BMS753, BMS961 and the like; and certain hydrophobic adenosine receptor agonists, such as UK-432,097.

Alternatively, immunostimulants and/or Treg promoting immunomodulators that are poorly water soluble, i.e., hydrophobic drug molecules, may be linked to the hydrophobic block (H) of the amphiphiles and/or peptide antigen conjugates (e.g., S-[B]-[U]-H-D and/or [S]-[E1]-A-[E2]-[U]-H-D) and may therefore be incorporated into the core of the particles comprising the amphiphiles and/or peptide antigen conjugates through covalent bonds between the drugs and the hydrophobic block (H) comprising the particle core. Non-limiting examples include immunostimulants and Treg promoting immunomodulators that are (i) poorly water soluble (ii) suitable for covalent conjugation; (iii) have severe dose-limiting toxicities when used systemically and therefore require a delivery platform to restrict biodistribution; and/or (iv) require covalent attachment to the amphiphile and/or peptide antigen conjugate to ensure adequate co-delivery with the peptide antigen (A). Non-limiting examples include immunostimulants such as imidazoquinoline based agonists of TLR-7 and -8, as well as diABZI-based agonists of STING; certain, conjugatable heterocyclic aromatic inhibitors of mTOR/PI3K/AKT, such as Torin 2; certain histone deacetylases (HDACs), such as butyric acid; certain agonists of aryl hydrocarbon receptors (AHR), such as tryptamine, kynurenine, kynurenic acid, 5-hydroxy tryptophan, indol-3-acetic acid and ITE; substrates for IDO, such as tryptophan; agonists of retinoic acid receptors (RAR), such as all-trans retinoic acid, TTNPB (cas: 71441-28-6), AM580, BMS753, BMS961 and the like; and, certain adenosine receptor agonists, such as UK-432,097.

In some embodiments of vaccines for inducing tolerance, the hydrophobic block (H) of amphiphiles and/or peptide antigen conjugates comprises a poly(amino acid) of Formula I, wherein the poly(amino acid) of Formula I comprises reactive amino acids (N) linked to hydrophobic drug molecules and optionally hydrophobic amino acids (M). Non-limiting examples are provided here for clarity:

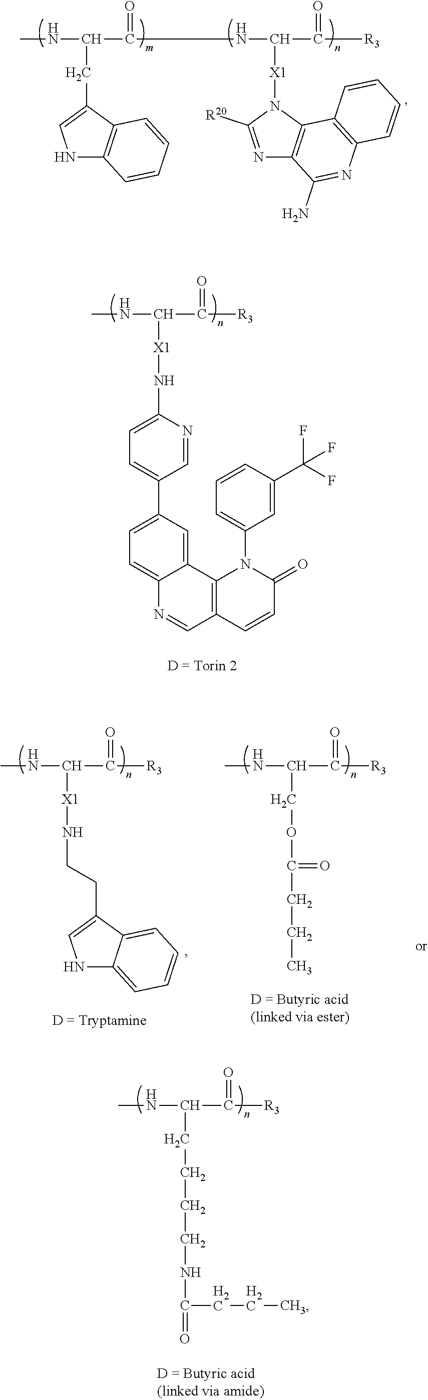

wherein X1 is any suitable linker and R³ is typically selected from hydrogen, NH₂, NH₂—CH₃, NH₂—(CH₂)ᵢCH₃, OH, or drug molecules (D) either linked directly or through any suitable linker molecule (X); n and m are any integers, wherein the sum of n and m (when present) is greater than 3, typically between about 3 and 30; and, the N-terminal amino acid is attached to either (i) a solubilizing block (S) either directly or indirectly via a spacer (B) and/or Linker U; (ii) an antigen (A) either directly or indirectly via an extension (E1 or E2) and/or Linker U; (iii) a drug molecule either directly or via a Linker U; or, (iv) a capping group.

In some embodiments, the drug molecule (D) is linked to the hydrophobic block (H) through an enzyme degradable peptide, which may further comprise a self-immolative linker. A non-limiting example is provided her for clarity:

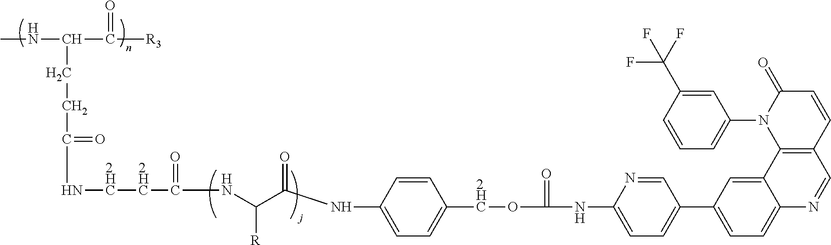

wherein j is any integer and R is any suitable amino acid composition, though, in preferred embodiments j is between 2 to 4 amino acids that are recognized by cathepsins.

Note: moderate to highly water soluble amphiphilic or hydrophilic drug molecules (D) may also be linked to the hydrophobic block (H) of the amphiphiles and/or peptide antigen conjugates (e.g., S-[B]-[U]-H-D and/or [S]-[E1]-A-[E2]-[U]-HD), but the solubilizing effects of the drug molecule (D) should be compensated for by the composition of the hydrophobic block. In some embodiments of vaccines for inducing tolerance, the hydrophobic block (H) of amphiphiles and/or peptide antigen conjugates comprises a poly(amino acid) of Formula I, wherein the poly(amino acid) of Formula I comprises hydrophobic amino acids (M) and reactive amino acids (N) linked to hydrophobic drug molecules.

In other embodiments of vaccines for inducing tolerance, the hydrophobic block (H) of amphiphiles and/or peptide antigen conjugates comprises a dendron, wherein the terminal functional groups are linked to hydrophobic drug molecules. A non-limiting example is provided here for clarity:

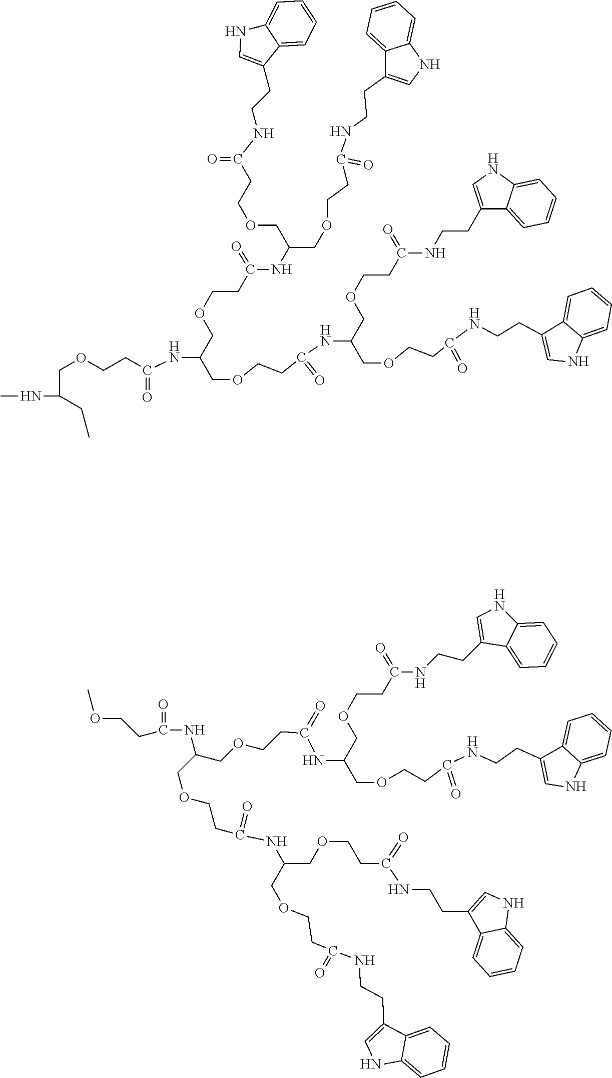 [SMILES: CCC(COCCC(=O)NC(COCCC(=O)NC(COCCC(=O)NCCc1c[nH]c2ccccc12)COCCC(=O)NCCc1c[nH]c2ccccc12)COCCC(=O)NC(COCCC(=O)NCCc1c[nH]c2ccccc12)COCCC(=O)NCCc1c[nH]c2ccccc12)NC ; COCCC(=O)NC(COCCC(=O)NC(COCCC(=O)NCCc1c[nH]c2ccccc12)COCCC(=O)NCCc1c[nH]c2ccccc12)COCCC(=O)NC(COCCC(=O)NCCc1c[nH]c2ccccc12)COCCC(=O)NCCc1c[nH]c2ccccc12]

wherein the drug molecule is tryptamine.

In preferred embodiments of vaccines for inducing tolerance wherein the immunostimulants and/or Treg promoting immunomodulators are moderate or highly water soluble, i.e., amphiphilic or hydrophilic drug molecules, the immunostimulants and/or Treg promoting immunomodulators are linked to hydrophobic blocks (H) to yield drug molecule conjugates (e.g., D-H or H-D) that may be admixed with amphiphiles and/or peptide antigen conjugates (e.g., D-H+S-[B]-[U]-H and/or [S]-[E1]-A-[E2]-[U]-H) and incorporated into the hydrophobic core of particles comprising the amphiphiles and/or peptide antigen conjugates through non-covalent interactions. Immunostimulants and Treg promoting immunomodulators that are water soluble and are preferred for attachment to hydrophobic molecules for admixing with amphiphiles and/or peptide antigen conjugates include but are not limited to peptide-based NLRs, such as muramyl dipeptide and any derivatives thereof; adenine-based agonists of TLR-7, as well as highly-water soluble nucleic acid-based agonists of TLR-3, TLR-7, TLR-9, STING and MDA5; certain agonists of aryl hydrocarbon receptor (AHR), such as kynurenine and kynurenic acid; moderately water soluble adenosine receptor agonists, e.g., agonists of A_{2A}, such as ATL-146e, YT-146, (N6-(2-(3,5-dimethoxyphenyl)-2-(2-methylphenyl)ethyl)adenosine (DPMA), regadenoson or zeatin; and protein and peptide-based agonists of TGF-β, IL-17, IL-2 and IL-10 receptors.

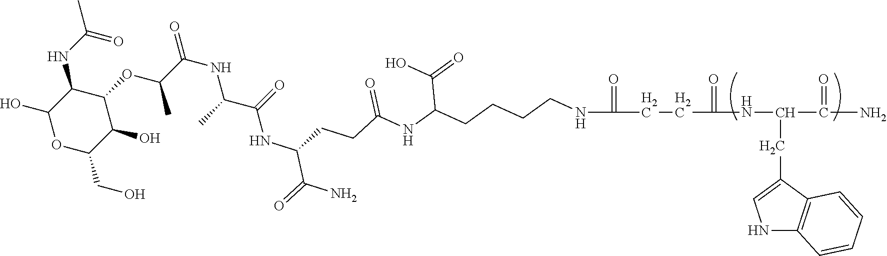

While admixing drug molecule conjugates (e.g., D-H or H-D) with amphiphiles and/or peptide antigen conjugates (e.g., D-H+S-[B]-[U]-H and/or [S]-[E1]-A-[E2]-[U]-H) is the preferred means to incorporate amphiphilic or hydrophilic drug molecules into the hydrophobic core of particles comprising the amphiphiles and/or peptide antigen conjugates for vaccines for inducing tolerance, such an approach is also effective for incorporating hydrophobic drug molecules, particularly when two or more different drug molecules are included in the vaccine for inducing tolerance.

An alternative means of incorporating nucleic acid-based drug molecules, such as nucleic acid-based agonists of TLR-3, TLR-7, TLR-9, STING and MDA5, includes electrostatic complexation with a positively charged hydrophobic block. Preferred compositions of amphiphiles and/or hydrophobic blocks for complexing nucleic acids are described elsewhere.

The inventors of the present disclosure identified compositions of vaccines for inducing tolerance that comprise specific, preferred combinations of drug molecules selected from immunostimulants and Treg promoting immunomodulators that led to unexpected improvements in the induction of regulatory T cells (Tregs) and/or trans-differentiation of Th1/Th2/Th17 cells to Tregs. Specifically, it was observed that, for vaccines for inducing tolerance comprising one or more peptide antigen conjugate of formula [S]-[E]-A-[E2]-[U]-H, an amphiphile of formula S-[B]-[U]-H and a drug molecule (D) comprising a Treg promoting immunomodulator selected from inhibitors of mTOR (e.g., Rapamycin, KU-0063794), RORγt (e.g., SR1555), CDK8/19 (e.g., CCT251921) and HDACs (e.g., SAHA, TMP269, etc.), as well as agonists of AHR (e.g., Kynurenine, Tryptamine, etc.), RAR (e.g., all-trans retinoic acid, BMS961, etc.) and A₂a (e.g., Zeatin or UK 432,097), and a second drug molecule (D2) comprising an immunostimulant selected from agonists of NLRs (e.g., muramyl dipeptide), CLRs (e.g., TDB), TLR-1, -2 and -6 (lipopeptides), TLR-4 (LPS and any derivatives thereof), TLR-7/8a (e.g., imidazoquinolines) and STING (e.g., CDNs, diABZI, etc.) that the magnitude of Tregs was lower than if the immunostimulant was not included. Non-limiting explanations are that the immunostimulant is needed to promote native T cell activation and/or T cell expansion, but that the Treg promoting immunomodulator blocks T cell differentiation towards T helper (Th) phenotypes.

Including immunostimulants in tolerance vaccines generally led to higher magnitude antigen-specific T cell responses; however, the portion of antigen-specific CD4 T cells with Treg phenotype (i.e., FOXP3 expression) depended on the specific combination of Treg promoting immunomodulators and immunostimulants present during T cell priming. For instance, vaccine compositions comprising antigens and immunostimulants generally induced CD4 T cells with Th1, Th2 and/or Th17 phenotypes. Induction of Tregs typically required the addition of a Treg promoting immunomodulator; however, the presence of both an immunostimulant and Treg promoting immunomodulator in vaccine compositions led to CD4 T cells with a distribution of phenotypes that depended on the specific compositions and combinations of immunostimulants and Treg promoting immunomodulators used.

Accordingly, the inventors of the present disclosure found that, when used in vaccines for inducing tolerance, immunostimulants that induced IL-12 and/or Type I IFNs, such as agonists of TLR-3, TLR-4, TLR-7, TLR-8, TLR-9, STING and MDA5 were highly Th1 polarizing and typically required the addition of Treg promoting immunomodulators selected from dual mTOR complex 1 (mTORC1) and mTOR complex 2 (mTORC2) inhibitors, including Torin 1 (Cas: 1222998-36-8), KU-0063794 (Cas: 938440-64-3) and omipalisib (Cas: 1086062-66-9), to block differentiation of CD4 T cells to Th1, Th2 and Th17 phenotypes, thus promoting CD4 T cell differentiation to Tregs. In contrast, immunostimulants that that induced lower or no IL-12 and/or Type I IFNs, such as agonists of CLRs, NLRs, TLR-1, TLR-2, TLR-5 and/or TLR-6 typically required the addition of Treg promoting immunomodulators selected from inhibitors of mTORC1 (e.g. Rapamcyin, Dactolisib, Everolimus and Temsirolimus, etc.), inhibitors of RORγt or agonists of AHR and/or RAR to promote CD4 T cell differentiation to Tregs. Thus, in preferred embodiments of vaccines for inducing tolerance comprising an immunostimulant, the vaccine further comprises at least one Treg promoting immunomodulator selected from dual inhibitors of mTORC1 and mTORC2, such as Torin 1, KU-0063794, and omipalisib. In still other embodiments of vaccines for inducing tolerance comprising an immunostimulant, wherein the immunostimulant is selected from agonists of CLRs, NLRs, TLR-1, TLR-2, TLR-5 and/or TLR-6, the vaccine further comprises at least one Treg promoting immunomodulator selected from dual inhibitors of mTORC1 and mTORC2, such as Torin 1, KU-0063794, and omipalisib, or inhibitors of mTORC1 (e.g. Rapamcyin, Dactolisib, Everolimus and Temsirolimus, etc.), inhibitors of RORγt or agonists of AHR and/or RAR.

### Compositions of Vaccines for Inducing Tolerance

In preferred embodiments of vaccines for inducing tolerance the vaccine comprises one or more, typically between 1 to 40, peptide antigen conjugates of formula [S]-[E1]-A-[E2]-[U]-H-[D](or H-[D]-U-[E1]-A-[E2]-[S]) and an amphiphile of formula S-[B]-[U]-H-[D], wherein each peptide antigen conjugate typically comprises an antigen (A) selected from autoantigens (or allergens), which is linked either directly or via an extension (E1 or E2) and/or Linker U to a hydrophobic block (H), which is typically selected from poly(amino acids) of Formula I comprising hydrophobic amino acids (M) and/or reactive amino acids (N) linked to drug molecules (D); and, additionally wherein the amphiphile is selected from amphiphiles with dendron or linear architecture comprising a solubilizing block (S) linked either directly or via a spacer (B) and/or Linker U to a hydrophobic block (H) typically selected from poly(amino acids) of Formula I comprising hydrophobic amino acids (M) and/or reactive amino acids (N) linked to drug molecules (D); and, wherein the amphiphile has dendron architecture the solubilizing block is selected from dendron amplifiers with between 2 and 32, more preferably between 4 and 8, solubilizing groups (SG) selected from carboxylic acid, phosphoserine (or glycerophosphoserine), glucose, mannose, glucosamine, n-acetylglucosamine, galactose, galactosamine, n-acetyl-galactosamine and/or agonists of CD22a, which may be linked either directly or indirectly via a linker (X) to the terminal functional groups (FGt) of the solubilizing block (S) with dendron architecture through any suitable means, though, in preferred embodiments the solubilizing group is linked to FGt via an amide bond. A non-limiting example is provided here for clarity:

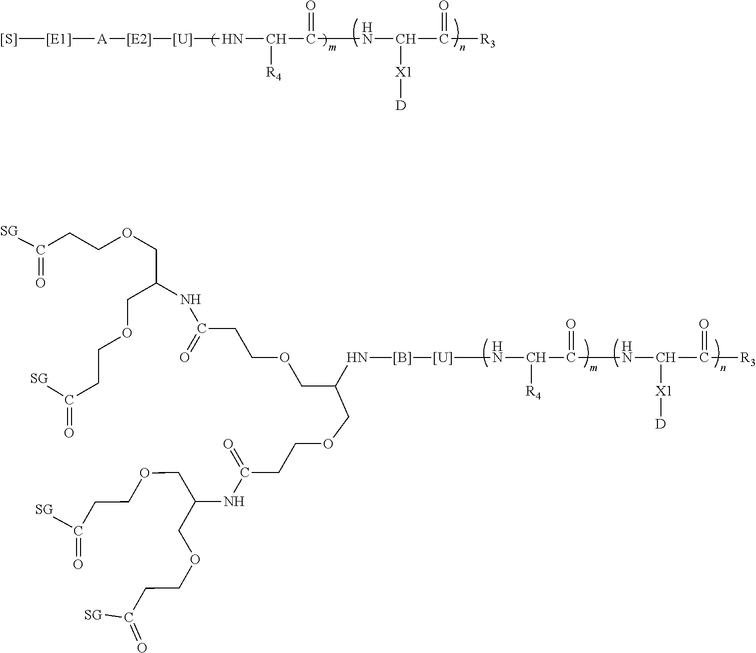

wherein X1 is any suitable linker molecule, D is any suitable drug molecule typically selected from immunostimulants and/or Treg promoting immunomodulators, R³ is typically selected from hydrogen, NH₂, NH₂—CH₃, NH₂—(CH₂)_{y5}CH₃, OH, or drug molecules (D) either linked directly or through any suitable linker molecule (X); m and n are any integers, wherein the sum of m and n (when present) is greater than 3, typically between about 3 and 30.

In preferred embodiments, the hydrophobic block (H) comprises poly(amino acids) of Formula I is typically selected from hydrophobic amino acids (M) selected from AHR agonists (e.g., kynurenine or 5HT) or IDO substrates (e.g., tryptophan) and/or reactive amino acids linked to hydrophobic immunomodulators (e.g., tryptamine). In the above example, wherein the hydrophobic block comprises hydrophobic amino acids selected from tryptophan, and wherein R³ is an amine, the structure is:

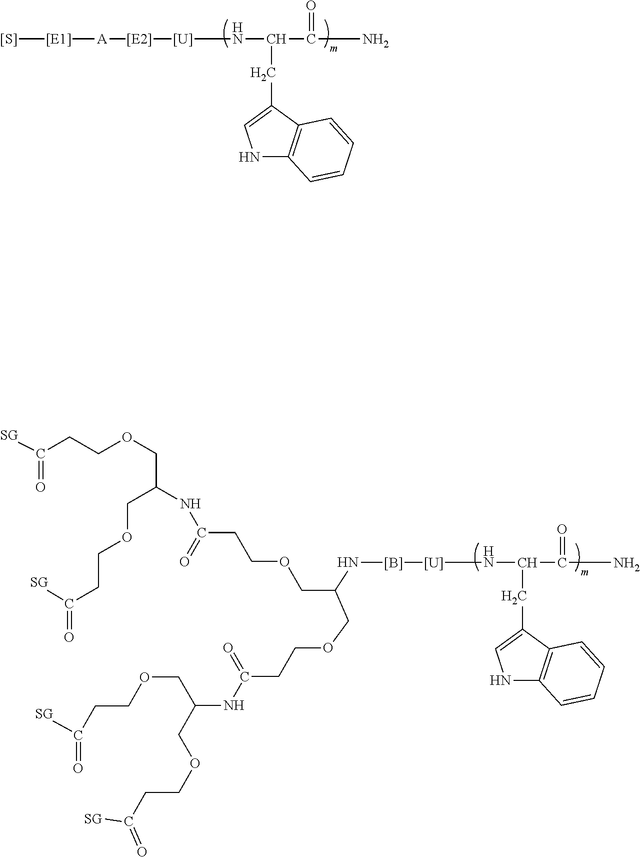

In some preferred embodiments of vaccines for inducing tolerance, the solubilizing group is selected from groups comprising carboxylic acids, mannose, phosphoserine (or glycerophosphoserine), glucose, glucosamine, n-acetylglucosamine, galactose, galactosamine, n-acetyl-galactosamine and/or agonists of CD22a, that are linked to S either directly or via a suitable linker X, or, more preferably, X5, typically selected from a lower alkyl or ethylene oxide linker. In the above example, wherein the solubilizing group comprises a carboxylic acid and is selected from beta-alanine and the spacer B is selected from PEG the structure is:

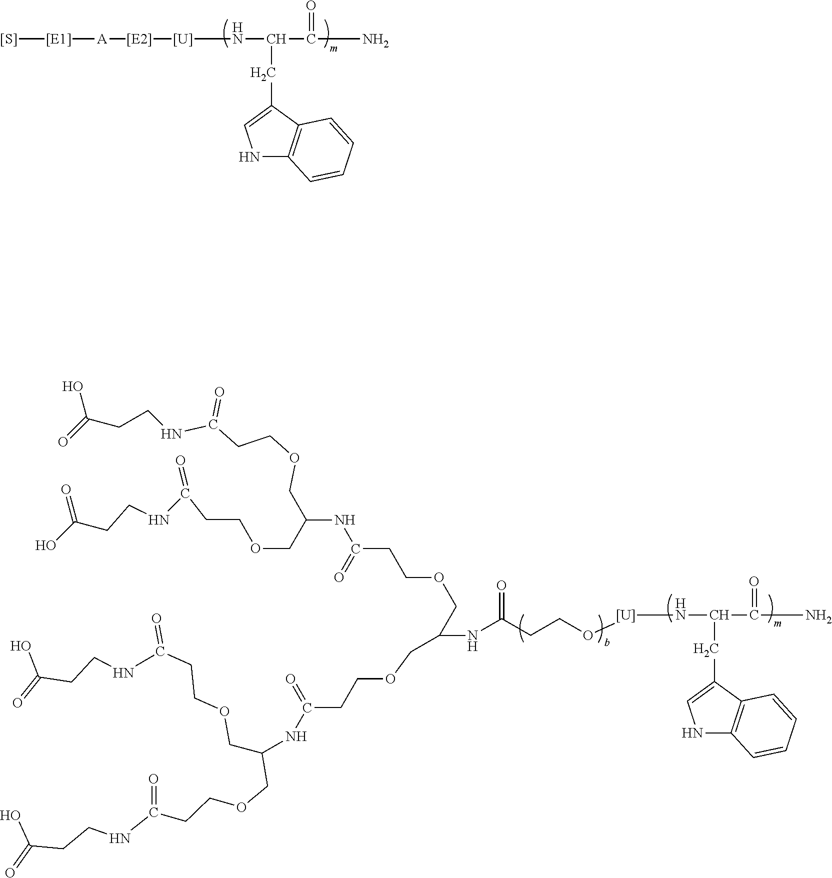

In the above example, wherein the solubilizing group comprises P3-GalNAc linked via X5 to the terminal functional group (FGt) of the dendron-based solubilizing block, the structure is:

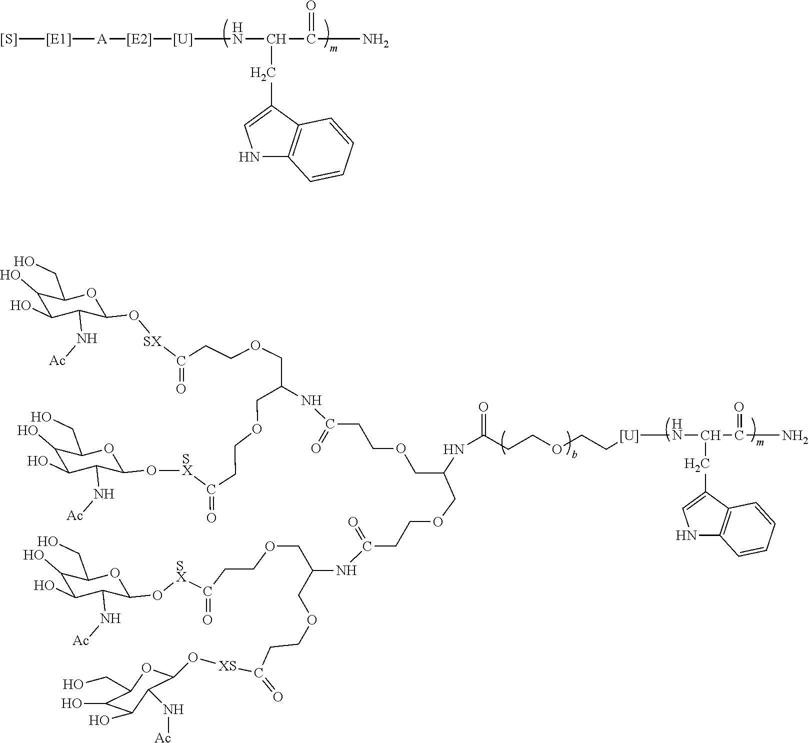

In the above example, wherein the solubilizing group comprises mannose linked via X5 to the terminal functional group (FGt) of the dendron-based solubilizing block, the structure is:

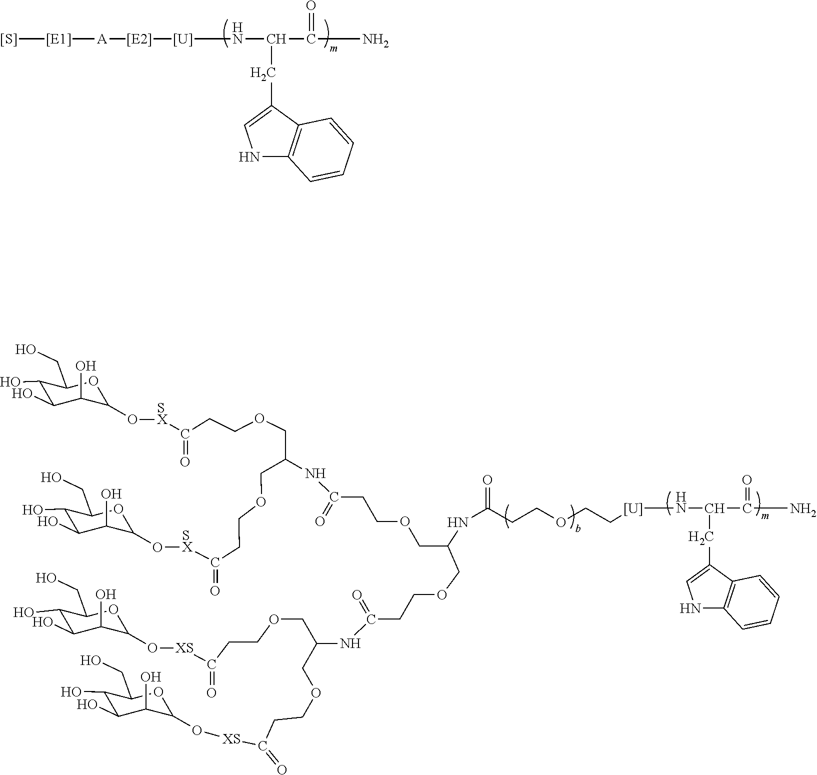

In certain preferred embodiments of tolerance vaccines, the peptide antigen conjugate comprises a solubilizing block that further comprises a poly(amino acid) selected from lysine and the above structure becomes:

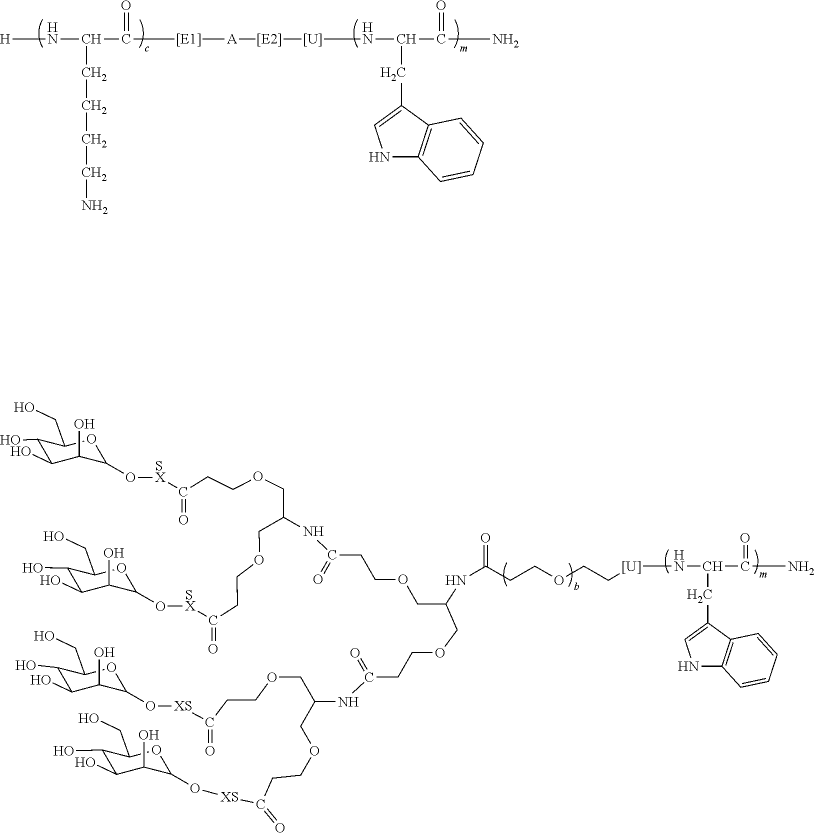

Wherein c represents an integer number of repeat units that is selected such that the peptide antigen conjugate net charge at physiologic pH is greater than 2, preferably between 2 and 6, such as 1, 2, 3, 4, 5 or 6, most preferably between 3 and 5, such as 3; A is a peptide antigen, E1 is an N-terminal extension, E2 is a C-terminal extension; m is typically between 3 and 30; X5 is a linker typically selected from PEG or short aliphatic groups and b is an integer number of repeating units typically selected from between about 4 to about 36 monomeric units, though, more preferably between about 12 to 24 units.

In some preferred embodiments of vaccines for inducing tolerance, the peptide antigen conjugate and/or amphiphile comprises a Linker U, preferably selected from linkers comprising a triazoline ring that results from the reaction of an azide with an alkyne. In the above example, wherein the peptide antigen conjugate and the amphiphile comprise a Linker U further comprising a triazole, a non-limiting example of a possible resulting structure is:

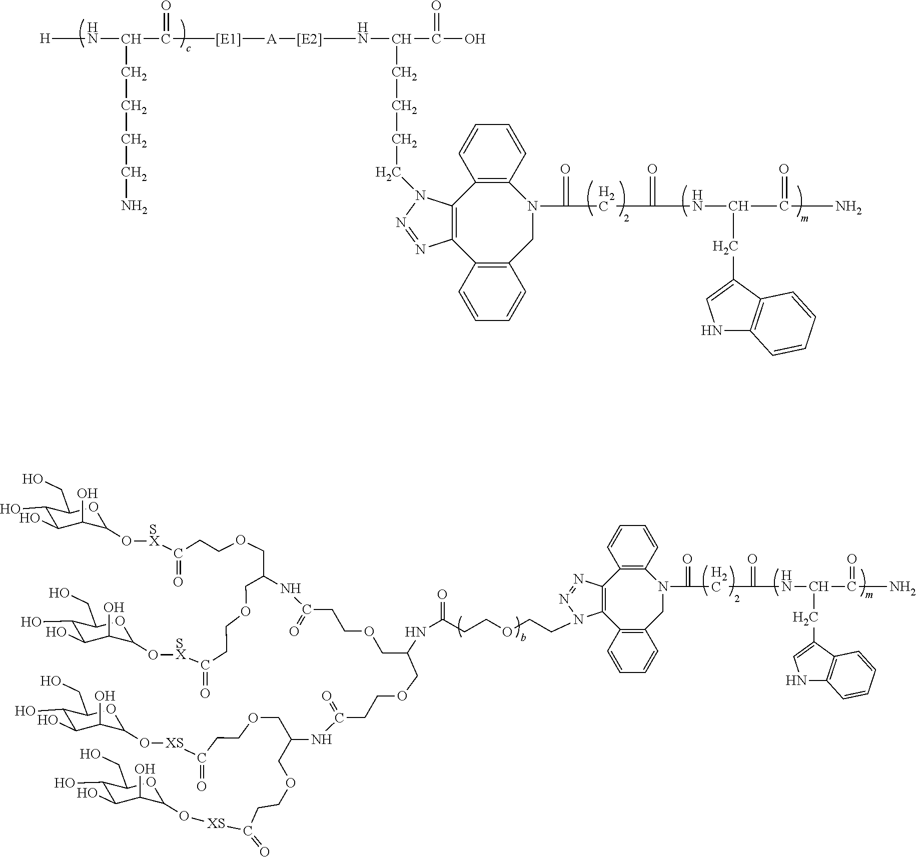

In still further preferred embodiments, the peptide antigen conjugate comprises an E1 N-terminal extension, typically selected from Val-Arg and an E2 C-terminal extension typically selected from Ser-Pro-Val-Cit and a X5 linker selected from PEG3, and the structure becomes:

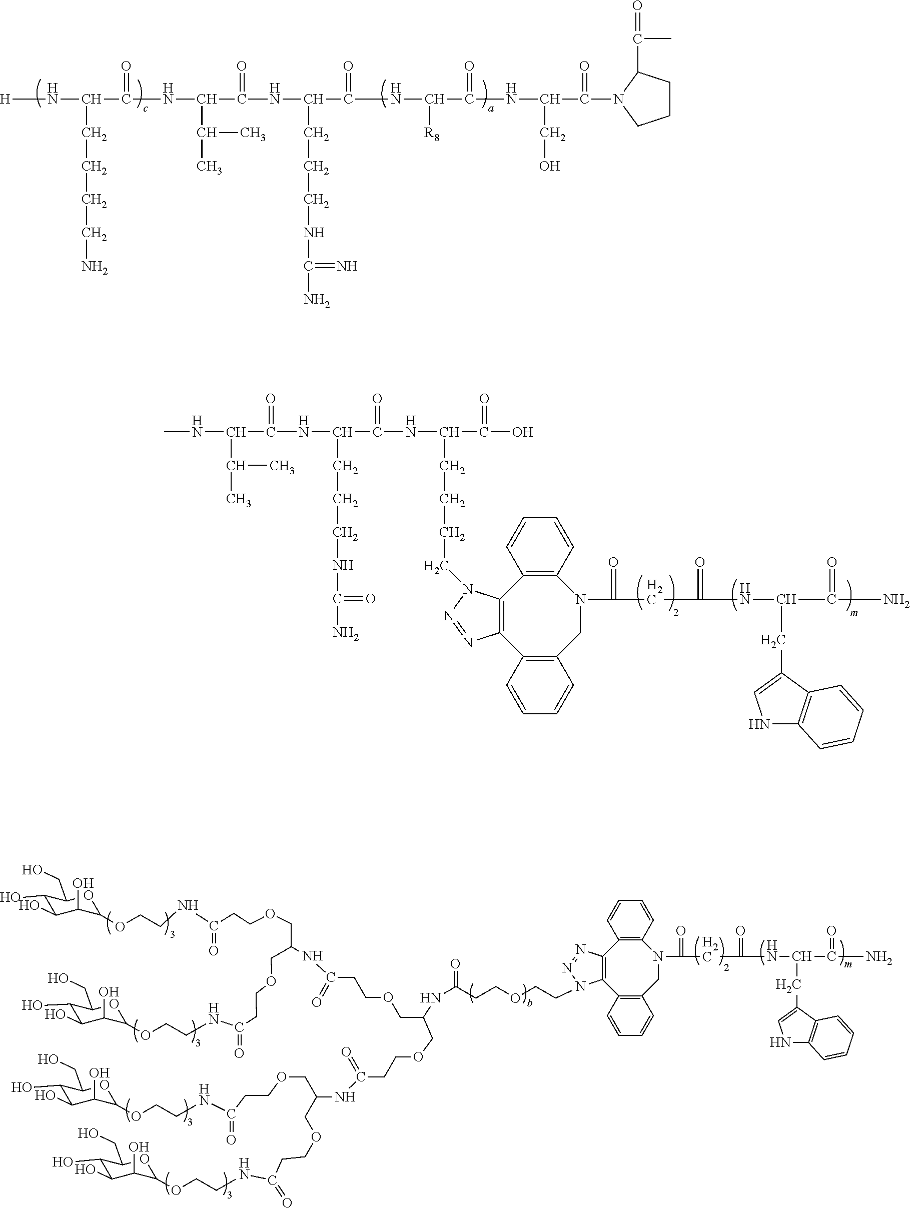

wherein c represents an integer number of repeat units that is selected such that the peptide antigen conjugate net charge at physiologic pH is greater than 2, preferably between 2 and 6, such as 1, 2, 3, 4, 5 or 6, most preferably between 3 and 5, such as 3; A is a peptide antigen with an integer, a, number of repeat units typically selected from 7 to 35, wherein R8 is any amino acid side chain; and b is an integer number of repeating units typically selected from between about 4 to about 36 monomeric units, though, more preferably between about 12 to 24 units. In certain preferred embodiments, the solubilizing group is GalNAc the above structure becomes:

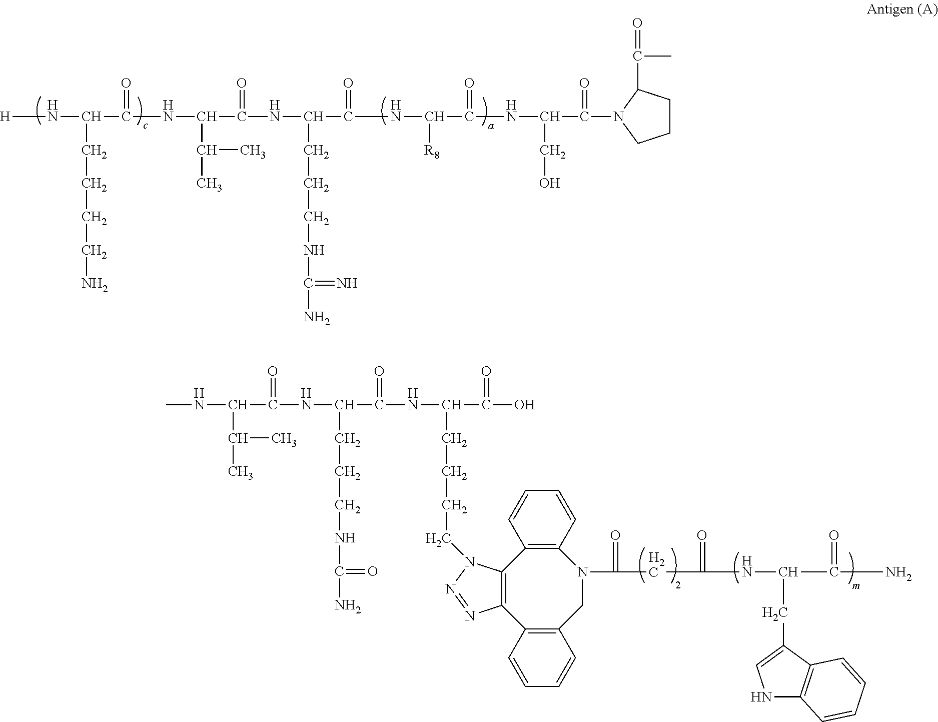

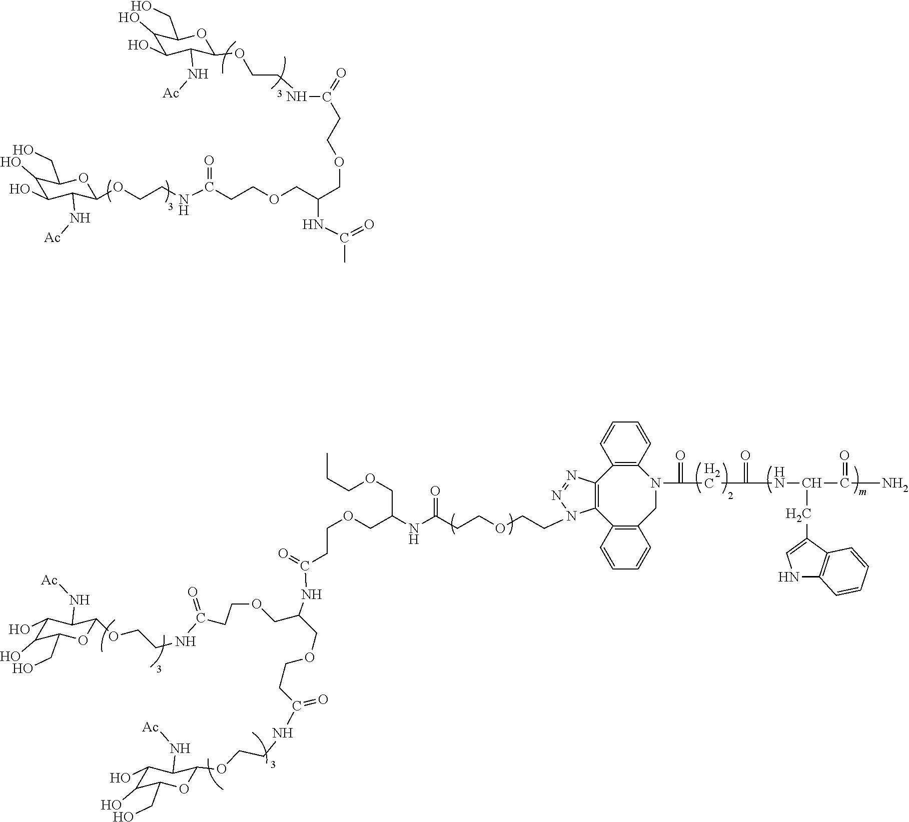

In still other embodiments, the solubilizing block comprises beta-alanine and the structure is:

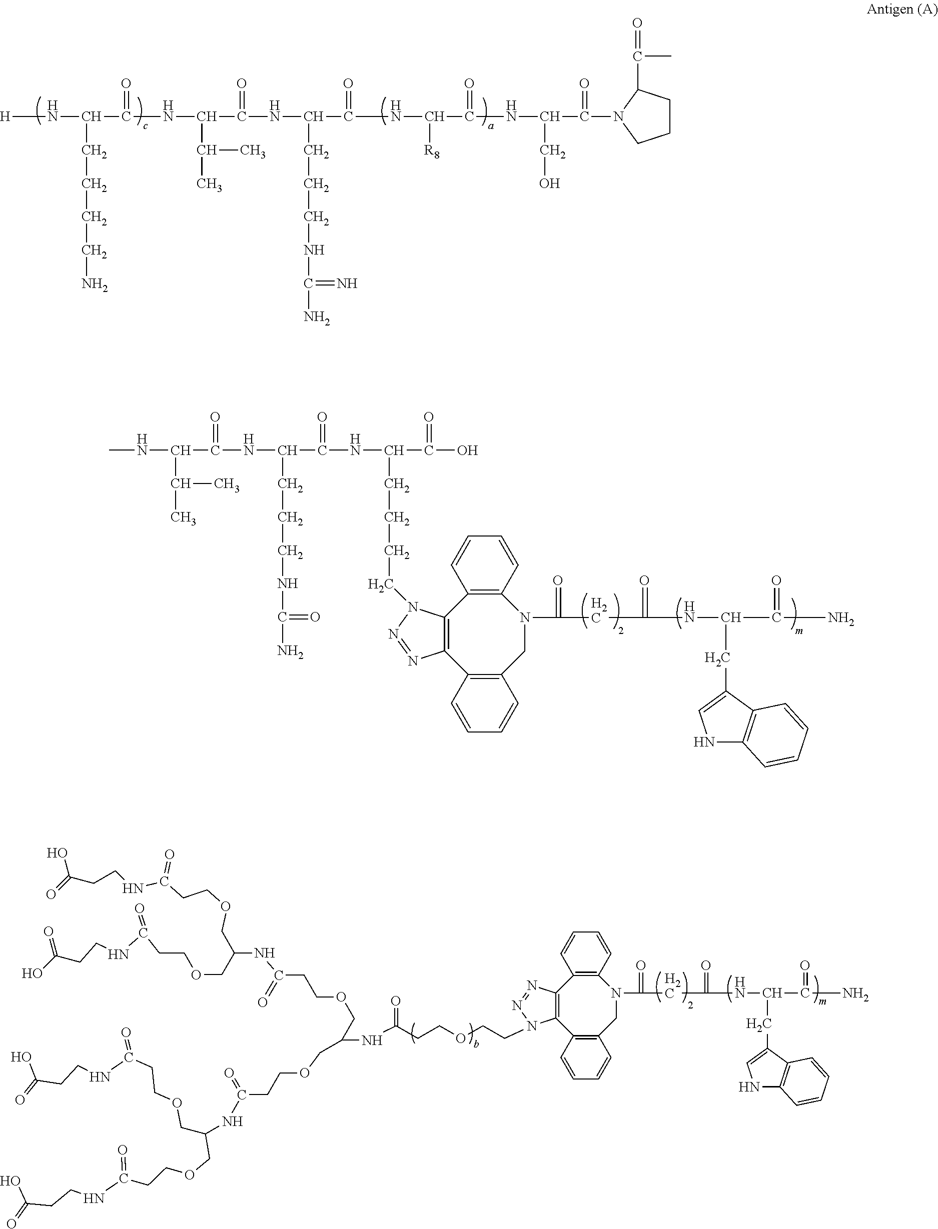

Or, the solubilizing block comprises succinic acid linked to a dendron-based amplifier and the structure is:

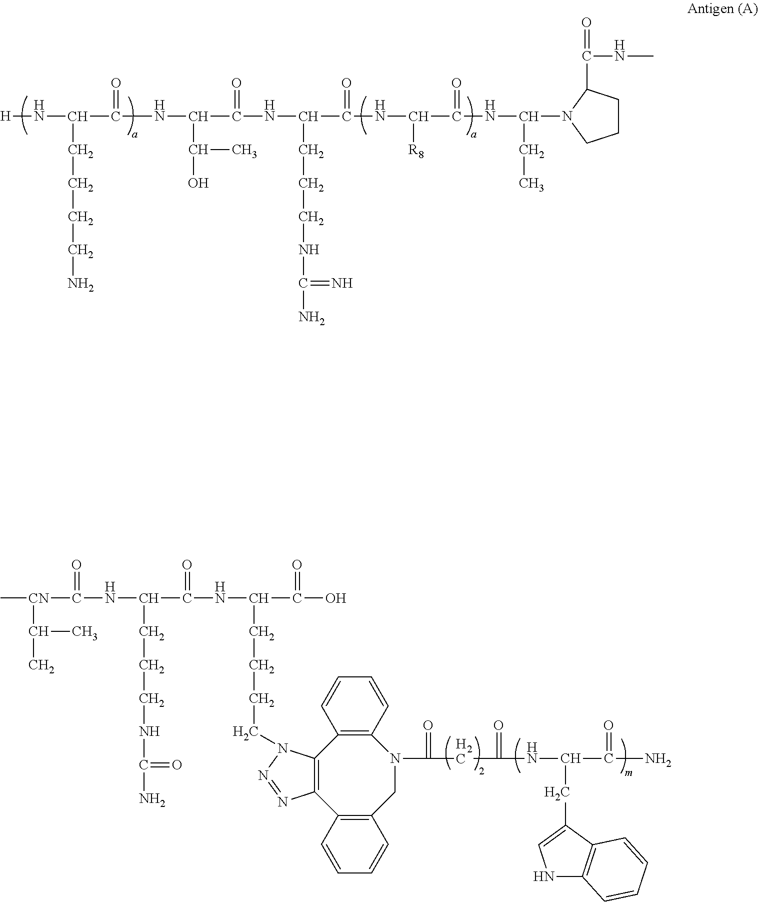

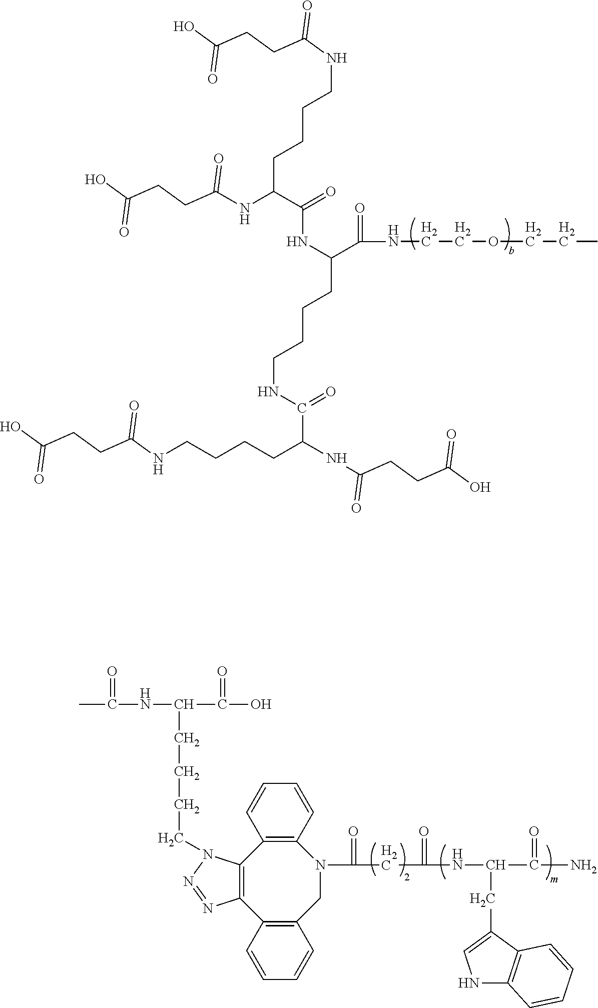

In some embodiments of tolerance vaccines, extensions (E1 or E2) are selected from heptad repeats of formula (AA_{H}-AA_{P}-AA_{P}-AA_{H}-AA_{P}-AA_{P}-AA_{P})ₑ. Preferred compositions of heptad repeats of formula (AA_{H}-AA_{P}-AA_{P}-AA_{H}-AA_{P}-AA_{P}-AA_{P})ₑ for use as extensions (E1 and/or E2) are described elsewhere in the specification.

In certain preferred embodiments of vaccines for inducing tolerance, the vaccine comprises one or more, typically no more than 40, peptide antigen conjugates of formula [S]-[E1]-A-[E2]-[U]-H-[D](or H-[D]-U-[E1]-A-[E2]-[S]), an amphiphile of formula S-[B]-[U]-H-[D] and a drug molecule (D) selected from inhibitors of mTOR (e.g., rapamycin) or agonists of AHR (e.g., kynurenine or ITE). In certain preferred embodiments of vaccines for inducing tolerance, the amphiphile is absent and the vaccine comprises one or more, typically no more than 40, peptide antigen conjugates of formula [S]-[E1]-A-[E2]-[U]-H-[D](or H-[D]-U-[E1]-A-[E2]-[S]) and a drug molecule (D) selected from inhibitors of mTOR (e.g., rapamycin) or agonists of AHR (e.g., kynurenine or ITE).

In still other preferred embodiments of vaccines for inducing tolerance, the vaccine comprises one or more, typically no more than 40, peptide antigen conjugates of formula [S]-[E1]-A-[E2]-[U]-H-[D](or H-[D]-U-[E1]-A-[E2]-[S]), an amphiphile of formula S-[B]-[U]-H-[D], a drug molecule (D) comprising a Treg promoting immunomodulator and a second drug molecule (D2) comprising an immunostimulant. In still other preferred embodiments of vaccines for inducing tolerance, the amphiphile is absent and the vaccine comprises one or more, typically no more than 40, peptide antigen conjugates of formula [S]-[E1]-A-[E2]-[U]-H-[D](or H-[D]-U-[E1]-A-[E2]-[S]), a drug molecule (D) comprising a Treg promoting immunomodulator and a second drug molecule (D2) comprising an immunostimulant. In preferred embodiments, the drug molecule (D) comprising a Treg promoting immunomodulator is selected from inhibitors of mTOR (e.g., rapamycin) or agonists of AHR (e.g., kynurenine) and the second drug molecule (D2) comprising an immunostimulant is selected from muramyl dipeptide (MDP), TDB, TLR4 agonists (e.g., MPL or LPS), lipopeptide TLR-1-2 and -6 agonists (e.g., Pam2Cys or Pam3Cys), or TLR-7 agonists, provided that if D2 is selected from agonists TLRs, the Treg promoting immunomodulator is selected from inhibitors of mTOR that inhibit both mTORC1 and mTORC2, such as such as Torin 1, KU-0063794, and omipalisib. Non-limiting exemplary combinations of D and D2 that fit these criteria include but are not limited to: (a) rapamycin and MDP, (b) rapamycin and TDB, (c) ITE and MDP, (d) ITE and TDB, (e) Torin1 and MPL, (f) Torin 1 and Pam2Cys and (g) Torin 1 and an imidazoquinoline.

In still other preferred embodiments of vaccines for inducing tolerance, the vaccine comprises one or more, typically no more than 40, peptide antigen conjugates of formula [S]-[E1]-A-[E2]-[U]-H-[D](or H-[D]-U-[E1]-A-[E2]-[S]), an amphiphile of formula S-[B]-[U]-H-[D], a drug molecule (D) comprising a Treg promoting immunomodulator and a second drug molecule (D2) comprising a Treg promoting immunomodulator. In still other preferred embodiments of vaccines for inducing tolerance, the amphiphile is absent and the vaccine comprises one or more, typically no more than 40, peptide antigen conjugates of formula [S]-[E1]-A-[E2]-[U]-H-[D](or H-[D]-U-[E1]-A-[E2]-[S]), a drug molecule (D) comprising a Treg promoting immunomodulator and a second drug molecule (D2) comprising a Treg promoting immunomodulator. In preferred embodiments, the drug molecule (D) comprising a Treg promoting immunomodulator is selected from an inhibitor of mTOR (e.g., rapamycin) or an inhibitor RORγt (e.g., SR1555) and the second drug molecule (D2) comprising a Treg promoting immunomodulator is selected from an agonist of AHR (e.g., kynurenine), RAR (e.g., retinoic acid) or A2a (e.g., zeatin or UK 432,097), or an inhibitor of HDACs (e.g., SAHA, TMP269, etc.). Non-limiting exemplary combinations of D and D2 that fit these criteria include but are not limited to: (a) rapamycin and kynurenine, (b) rapamycin and ITE, (c) rapamycin and retinoic acid, (d) rapamycin and SAHA.

In some additional embodiments, both the drug molecule (D) and second drug molecule (D2) comprise a Treg promoting immunomodulator selected from agonists of AHR (e.g., Kynurenine), RAR (e.g., retinoic acid) or A2a (e.g., Zeatin or UK 432,097), or an inhibitor of HDACs (e.g., SAHA, TMP269, etc.).

In still other preferred embodiments of vaccines for inducing tolerance, the vaccine comprises one or more, typically no more than 40, peptide antigen conjugates of formula [S]-[E1]-A-[E2]-[U]-H-[D](or H-[D]-U-[E1]-A-[E2]-[S]), optionally an amphiphile of formula S-[B]-[U]-H-[D], a first drug molecule (D) a second drug molecule (D2) and a third drug molecule, (D3), wherein D D2 and D3 comprise Treg promoting immunomodulators independently selected from AHR (e.g., Kynurenine), RAR (e.g., retinoic acid) or A2a (e.g., Zeatin or UK 432,097), or an inhibitor of HDACs (e.g., SAHA, TMP269, etc.). In some additional embodiments, D is selected from inhibitors of mTOR (e.g., rapamycin) or RORγt (e.g., SR1555) and D2 and D3 are each independently selected from agonists of AHR (e.g., Kynurenine), RAR (e.g., retinoic acid) or A2a (e.g., Zeatin or UK 432,097), or an inhibitor of HDACs (e.g., SAHA, TMP269, etc.). Non-limiting exemplary combinations of D, D2 and D3 that fit these criteria include but are not limited to: (a) kynurenine, retinoic acid and SAHA and (b) rapamycin, kynurenine and SAHA.

In still other preferred embodiments of vaccines for inducing tolerance, the vaccine comprises one or more, typically no more than 40, peptide antigen conjugates of formula [S]-[E1]-A-[E2]-[U]-H-[D](or H-[D]-U-[E1]-A-[E2]-[S]), optionally an amphiphile of formula S-[B]-[U]-H-[D], a drug molecule (D) comprising an immunostimulant, a second drug molecule (D2) comprising a Treg promoting immunomodulator and a third drug molecule (D3) comprising a Treg promoting immunomodulator. In preferred embodiments, D1 is selected from agonists of NLRs (e.g., muramyl dipeptide), CLRs (e.g., TDB), TLR-1, -2 and -6 (lipopeptides), TLR-4 (LPS and any derivatives thereof), TLR-7/8a (e.g., imidazoquinolines) and STING (e.g., CDNs, diABZI, etc.), and D2 and D3 are selected from inhibitors of mTOR (e.g., rapamycin) and RORγt (e.g., SR1555). In some embodiments, D1 is selected from agonists of NLRs (e.g., muramyl dipeptide), CLR (e.g., TDB), TLR-1, -2 and -6 (lipopeptides), TLR-4 (LPS and any derivatives thereof), TLR-7/8a (e.g., imidazoquinolines) and STING (e.g., CDNs, diABZI, etc.), D2 is selected from inhibitors of mTOR (e.g., rapamycin) and RORγt (e.g., SR1555) and D3 is selected from agonists of AHR (e.g., Kynurenine), RAR (e.g., retinoic acid) or A2a (e.g., Zeatin or UK 432,097), or an inhibitor of HDACs (e.g., SAHA, TMP269, etc.). In still other embodiments, D1 is selected from agonists of NLRs (e.g., muramyl dipeptide), CLRs (e.g., TDB), TLR-1, -2 and -6 (lipopeptides), TLR-4 (LPS and any derivatives thereof), TLR-7/8a (e.g., imidazoquinolines) and STING (e.g., CDNs, diABZI, etc.), and both D2 and D3 are each independently selected from agonists of AHR (e.g., Kynurenine), RAR (e.g., retinoic acid) or A2a (e.g., Zeatin or UK 432,097), or inhibitors of HDACs (e.g., SAHA, TMP269, etc.). Non-limiting exemplary combinations of D, D2 and D3 that fit these criteria include but are not limited to: (a) TDB, rapamycin and SR1555, (b) TDB, rapamycin and kynurenine, (c) an imidazoquinoline, Torin 1 and kynurenine, (d) TDB, kynurenine and retinoic acid and (e) TDB, kynurenine and SAHA.

In still other preferred embodiments of vaccines for inducing tolerance, the vaccine comprises one or more, typically no more than 40, peptide antigen conjugates of formula [S]-[E1]-A-[E2]-[U]-H-[D](or H-[D]-U-[E1]-A-[E2]-[S]), optionally an amphiphile of formula S-[B]-[U]-H-[D], a drug molecule (D) comprising an immunostimulant, a second drug molecule (D2) comprising an immunostimulant and a third drug molecule (D3) comprising a Treg promoting immunomodulator. In preferred embodiments, D1 and D2 are each independently selected from agonists of NLRs (e.g., muramyl dipeptide), CLRs (e.g., TDB), TLR-1, -2 and -6 (lipopeptides), TLR-4 (LPS and any derivatives thereof), TLR-7/8a (e.g., imidazoquinolines) and STING (e.g., CDNs, diABZI, etc.), and D3 is selected from inhibitors of mTOR (e.g., rapamycin) and RORγt (e.g., SR1555). Non-limiting exemplary combinations of D, D2 and D3 that fit these criteria include but are not limited to (a) TDB, LPS and torin1 and (b) MDP, TDB and torin1.

An unexpected finding reported herein is that Treg promoting immunomodulators selected from inhibitors that inhibit both mTORC1 and mTORC2 included in vaccines for inducing tolerance led to a higher proportion of CD4 T cells with Treg phenotype as compared with use of inhibitors that solely inhibit signalling downstream of mTORC1. Moreover, vaccines for inducing tolerance comprising Treg promoting immunomodulators selected from inhibitors that inhibit both mTORC1 and mTORC2 and immunostimulants that induce IL-12 and/or IFNs, including agonists of TLR-3, TLR-7, TLR-8, TLR-9, RIGI and STING, were found to increase the number as well as proportion of CD4 T cells with Treg phenotype as compared with vaccines for inducing tolerance that lacked the immunostimulant. This is highly unexpected because immunostimulants that induce IL-12 and IFNs are considered some of the most potent immunostimulants for use as vaccine adjuvants for inducing cytotoxic T cells. Thus, it was highly unexpected and seemingly paradoxical that such immunostimulants could be included in a vaccine for inducing tolerance for inducing regulatory T cells.

Based on these findings, certain preferred compositions of vaccines for inducing tolerance comprise Treg promoting immunomodulators selected from inhibitors of both mTORC1 and mTORC2 and optionally an immunostimulant selected from TLR-3, TLR-7, TLR-8, TLR-7/8, TLR-9, RIGI and STING.

In preferred embodiments of vaccines for inducing tolerance, the Treg promoting immunomodulator is selected from an ATP-competitive mTOR inhibitor that inhibits signalling downstream of both mTORC1 and mTORC2. Non-limiting examples of ATP-competitive mTOR inhibitors include those described in US2008/0081809A1. Non-limiting examples include AZD-8055, AZD2016, KU-0063794. Additional examples of ATP-competitive inhibitors include those of the pyrazino(2,3-b)pyrazine class such as the molecules described in U.S. Pat. No. 8,492,381 B2, including CC223. In certain preferred embodiments, the ATP-competitive inhibitor is a benzonaphthridinone as described in U.S. Pat. No. 8,394,818 B2 and by Liu and colleagues (Liu, et al. J. Med. Chem. 2010. Non-limiting examples of benzonaphthridinone class molecules includes Torin-1 and Torin-2. Other examples include pyrazolopyrimidine analogs, including those described in US 2008/0234262 A1, such as WYE354 and WYE132; fused bicyclic mTOR inhibitors as described in U.S. Pat. No. 8,796,455 B2, including OSI-027 and OXA-01; and other ATP-competitive inhibitors such as PP242, PI-103, NVP-BEZ235, GNE-493 and GSK2126458.

In preferred embodiments of vaccines for inducing tolerance comprising a Treg promoting immunomodulator and an immunostimulant, the Treg promoting immunomodulator is an ATP-competitive inhibitor selected from AZD-8055, AZD2016, KU-0063794, CC223, Torin-1, Torin-2, INK-128, WYE354, WYE132, OSI-027, OXA-01, PI-103, NVP-BEZ235, GNE-493 and GSK2126458, and the immunostimulant is an agonist of TLR-3, TLR-3, TLR-7, TLR-8, TLR-7/8, TLR-9, RIGI and STING. In non-limiting examples of a vaccine for inducing tolerance comprising a Treg promoting immunomodulator and an immunostimulant, the Treg promoting immunomodulator is Torin-1 and the immunostimulant is an imidazoquinoline TLR-7, TLR-8 and/or TLR-7/8 agonist.

For vaccines comprising peptide antigen conjugates and Treg promoting immunomodulators selected from inhibitors of mTOR, the molar ratio of total peptide antigen conjugate (i.e., the total molar amount of peptide antigen conjugate) to Treg promoting immunomodulator was found to have a significant impact on particle hydrodynamic behavior as well as toxicity and efficacy. Accordingly, it was observed that molar ratio of total peptide antigen conjugate (i.e., the total molar amount of peptide antigen conjugate) to inhibitor of mTOR from about 100:1 to about 1:4 were suitable with molar ratios of about 10:1 to 1:4 being preferred and molar ratios of about 5:1 to about 1:3, such as 5:1, 4:1, 3:1, 2:1, 1:1, 1:2 and 1:3 more preferred, and molar ratios of about 5:1 to about 1:2 or about 5:1 to about 1:1 or about 4:1 to about 2:1 being most preferred. In some embodiments wherein the Treg promoting immunomodulator is linked to the peptide antigen conjugate, the preferred molar ratio of peptide antigen conjugate to Treg promoting immunomodulators selected from inhibitors of mTOR is about 1:1 to about 1:3, such as 1:1, 1:2 or 1:3.

The unexpected finding that vaccines for inducing tolerance comprising Treg promoting immunomodulators selected from inhibitors of both mTORC1 and mTORC2 and agonists of TLR-3, TLR-7, TLR-8, TLR-9, RIGI and STING increase the number as well as proportion of CD4 T cells with Treg phenotype provides the possibility of using expression systems for encoding antigens, including DNA, RNA, and viruses as platforms for tolerance induction. For instance, RNA expressions systems can be used to encode antigens for use as vaccines, but certain compositions of RNA can induce cytotoxic T cell responses through binding to TLR-7 (Diebold et al. Science. 2004; and Kranz et al. Nature. 2016). However, the results disclosed herein provide a possibility of modulating the immunostimulatory capacity of RNA and DNA-based expression systems through co-delivery of combined inhibitors of mTORC1 and mTORC2. Thus, in preferred embodiments of vaccines for inducing tolerance comprising RNA, DNA or viral vector-based expression systems, the tolerance vaccine comprises an ATP-competitive inhibitor that inhibits mTORC1 and mTORC2 signaling. Non-limiting examples include but are not limited to AZD-8055, AZD2016, KU-0063794, CC223, Torin-1, Torin-2, WYE354, WYE132, OSI-027, OXA-01, PI-103, NVP-BEZ235, GNE-493 and GSK2126458.

Peptide Antigen Conjugates and/or Peptide Antigen Fragments for Activating, Priming and/or Expanding T Cells and CAR-T Cells In Vitro or Ex Vivo

T cells and CAR-T cells may be activated, primed and/or expanded outside of a subject in vitro or ex vivo. The T cells may occur as a mixed leukocyte culture isolated from a subject, e.g., the T cells may be present within a population of blood cells (e.g., PBMCs), bone marrow cells and/or tumor-infiltrating leukocytes derived from a subject, e.g., a patient. Alternatively, the T cells may be isolated from a mixed population and/or may be enriched for a specific T cell subset, e.g., CD4 or CD8 T cells. The T cells may be clonal or poly(clonal) and/or may be genetically altered, e.g., such as a CAR-T cell.

Activating, priming and/or expanding T cells in vitro or ex vivo often involves the addition of peptide antigens to, or expression within, the cell culture. The peptide antigens are often selected to activate, prime and/or expand T cells that recognize specific peptide-MHC complexes. For activating, priming and/or expanding T cells in cell culture, the peptide antigens must be manufacturable and water soluble, and preferably should not be prone to oxidation. However, many peptide antigens that comprise epitopes that bind to MHC are enriched with hydrophobic residues that reduce solubility and present challenges to manufacturing.

To address this challenge, solubilizing blocks were introduced to peptide antigen conjugates of formula S-[E1]-A-[E2]-[U]-H-[D] and [D]-H-[E1]-A-[E2]-[U]-S and peptide antigen fragments of formula S-[E1]-A-[E2]-[U1] and [U1]-[E1]-A-[E2]-S, to improve solubility of sequences comprising peptides during manufacturing and for use activating, priming and/or expanding T cells in vitro or ex vivo.

Our results showed that solubilizing blocks comprising positively charged amino acids preferably selected from lysine, ornithine and arginine improved solubility and manufacturing of the peptide antigen conjugates of formula S-[E1]-A-[E2]-[U]-H-[D] and [D]-H-[E1]-A-[E2]-[U]-S and peptide antigen fragments of formula S-[E1]-A-[E2]-[U1] and [U1]-[E1]-A-[E2]-S. Therefore, in preferred embodiments of peptide sequences for activating, priming and/or expanding T cells, peptide sequences are selected from peptide antigen conjugates of formula S-[E1]-A-[E2]-[U]-H-[D] and [D]-H-[E1]-A-[E2]-[U]-S and peptide antigen fragments of formula S-[E1]-A-[E2]-[U1] and [U1]-[E1]-A-[E2]-S, the peptide sequence comprises a solubilizing block further comprising positively charged amino acids, preferably selected from lysine, ornithine or arginine.

A notable finding was that the site of attachment of any modifications to the peptide antigen impacted T cell recognition in vitro. Accordingly, modifications to the C-terminus of peptide antigens were generally well tolerated, whereas modifications to the N-terminus were less well tolerated, i.e., were less potent for recognition by T cells. Based on these findings, preferred embodiments of peptide sequences used for activating, priming and/or expanding T cells in vitro or ex vivo are typically selected from peptide sequences with the solubilizing block (S) linked to the C-terminus of the peptide antigen either directly or indirectly through an extension (E2), such as peptide antigen conjugates of formula [D]-H-[U]-[E1]-A-[E2]-S and peptide antigen fragments of formula [U1]-[E1]-A-[E2]-S, wherein S comprises positively charged amino acids, preferably selected from lysine, ornithine or arginine.

The extensions were also found to impact T cell recognition of the peptide antigen (A). Accordingly, peptide sequences comprising peptide antigens (A) linked to extensions (E1 and/or E2) selected from cathepsin cleavable tetrapeptides of the formula P4-P3-P2-P1 were generally more potent than those with shorter extensions, e.g., dipeptides, P2-P1. Non-limiting examples of tetrapeptide extensions suitable for use with peptide antigens used for activating, priming and/or expanding T cells in vitro or ex vivo include but are not limited Ser-Pro-Val-Arg (SEQ ID NO:10), Ser-Pro-Val-Cit (SEQ ID NO:8), Ser-Pro-Val-aBut (aBut=alpha-aminobutyric acid). A detailed description of other preferred compositions of cathepsin cleavable tetrapeptides is described elsewhere.

A final notable finding was that replacement of methionine and cysteine residues of peptide antigens (A) with norleucine and alpha-amino butyric, respectively, resulted in improved manufacturing without altering T cell recognition of the peptide antigen. Therefore, in preferred embodiments of peptide antigens used for activating, priming and/or expanding T cells in vitro or ex vivo, any methionine and cysteine amino acids (or “residues”) are replaced with norleucine and alpha-amino butyric acid, respectively.

Preferred embodiments of peptide sequences used for activating, priming and/or expanding T cells in vitro or ex vivo are typically selected from peptide antigen conjugates of formula S-[E1]-A-[E2]-[U]-H-[D] and [D]-H-[E1]-A-[E2]-[U]-S and peptide antigen fragments of formula S-[E1]-A-[E2]-[U1] and [U1]-[E1]-A-[E2]-S, wherein S comprises one or more amino acids, typically between 2 and 12, such as, 2, 3, 4, 5, 6, 7, 8, 9, 10, 11, or 12, preferably between 2 and 8, more preferably between 4 and 6 amino acids, typically selected from lysine, arginine and ornithine; E1 and E2 when present are selected from cathepsin cleavable tetrapeptides of the formula P4-P3-P2-P1, preferably selected from Ser-Pro-Val-Arg (SEQ ID NO: 10), Ser-Pro-Val-Cit (SEQ ID NO:8) and Ser-Pro-Val-aBut (aBut=alpha-aminobutyric acid); and methionine and cysteine residues of naturally occurring peptide antigens are optionally replaced with norleucine and alpha-aminobutyric acid, respectively.

A non-limiting example of a peptide sequence used for activating, priming and/or expanding T cells in vitro or ex vivo is shown here for clarity: [U1]-[E1]-A-Ser-Pro-Val-Arg-(Lys)₂₋₁₂, wherein in preferred embodiments U1 and E1 are absent and the sequence is A-Ser-Pro-Val-Arg-(Lys)₂₋₁₂, wherein A is any peptide antigen, typically between 7 and 35 amino acids in length, and the number of Lysine residues is typically selected from 2-12, more preferably 4, 5 or 6, such as A-Ser-Pro-Val-Arg-(Lys)₄, A-Ser-Pro-Val-Arg-(Lys)s, or A-Ser-Pro-Val-Arg-(Lys)₆.

### Peptide Antigen Conjugates Used in Heterologous Prime-Boost Vaccine Regimens

Heterologous prime-boost vaccines comprise two or more vaccines that differ in composition, i.e., a first vaccine (V1) and a second vaccine (V2), wherein V1 and V2 are different. The first vaccine may be administered at a first time (V1T1) and may be optionally administered a second time (V1T2) or a third time (V1T3), etc. The second vaccine is administered after the last administration of the first vaccine a first time (V2T1) and may be optionally administered a second time (V2T2) or a third time (V2T3), etc. The amount of time between administrations is referred to as the interval and is typically selected to be between 1 week and 12 weeks for a heterologous prime-boost vaccine regimen, though, more preferably the interval is between 1 and 6 weeks, such as 1 week, 2 weeks, 3 weeks, 4 weeks, 5 weeks, or 6 weeks.

The composition of V1 and V2 are selected to maximize the immune response and are typically selected from viral vaccines (e.g., adenoviruses, vaccinia viruses, etc.), protein or peptide vaccines (e.g., vaccines comprising peptide antigen conjugates), and nucleic acid vaccines. In some embodiments, V1 comprises a protein or peptide vaccine and V2 comprise a viral vaccine. In some embodiments V1 and V2 comprise viral vaccines.

In some heterologous prime-boost vaccines, V1 comprises a peptide antigen conjugate, e.g., a peptide antigen conjugate of formula [S]-[E1]-A-[E2]-[U]-H-[(D)] or [(D)]-H-[U]-[E1]-A-[E2]-[S], and V2 comprises a viral vaccine. In other embodiments of heterologous prime-boost vaccines, V1 comprises a viral vaccine and V2 comprises a peptide antigen conjugate, e.g., a peptide antigen conjugate of formula [S]-[E1]-A-[E2]-[U]-H-[(D)] or [(D)]-H-[U]-[E1]-A-[E2]-[S].

The inventors of the present disclosure found that certain heterologous prime-boost vaccine compositions, e.g., heterologous prime-boost vaccines comprising V1 and V2 selected from vaccines comprising peptide antigen conjugates and adenoviruses, led to significantly enhanced T cell responses. In some embodiments, V1 is selected from a peptide antigen conjugate, e.g., a peptide antigen conjugate of formula [S]-[E1]-A-[E2]-[U]-H-[(D)] or [(D)]-H-[U]-[E1]-A-[E2]-[S], and V2 is selected from an adenovirus, e.g., ChAdOx, wherein the adenovirus encodes for the antigen (A) of V1. In other embodiments, V1 is selected from an adenovirus, e.g., ChAdOx, wherein the adenovirus encodes for the antigen (A) that is included in V2 comprising a peptide antigen conjugate, e.g., a peptide antigen conjugate of formula [S]-[E1]-A-[E2]-[U]-H-[(D)] or [(D)]-H-[U]-[E1]-A-[E2]-[S].

A notable finding disclosed herein is that the route of administration for heterologous prime-boost vaccines comprising peptide antigen conjugates and viral vectors had a major impact on the magnitude of T cell response induced against the antigen (A). An unexpected finding was that, following administration of V1 comprising a peptide antigen conjugate administered by either the IM or IV route, V2 comprising an adenovirus resulted in significantly higher magnitude T cell responses when administered by the IV route as compared with V2 administered by the IM route.

In preferred embodiments of heterologous-prime boost vaccines comprising V1 selected from protein or peptide vaccines (e.g., peptide antigen conjugates of formula [S]-[E1]-A-[E2]-[U]-H-[(D)] or [(D)]-H-[U]-[E1]-A-[E2]-[S]), optionally further comprising an amphiphile, e.g., an amphiphile of formula S-[B]-[U]-H-[(D)], and V2 selected from viral vaccines, more preferably adenoviruses, e.g., ChAdOx, V1 is administered by either the IM or IV route and V2 is administered by the IV route at least 1 week after administration of V1, more preferably between 1 and 12 weeks after V1, most preferably between 1 and 6 weeks after V1.

In some embodiments, a method of treating or preventing a cancer, an autoimmune disease, an allergy, an infectious disease, a cardiovascular disease, or a neurodegenerative disease in a subject in need thereof comprising administering to the subject any of the vaccines disclosed herein.

### EXAMPLES

### Example 1: Synthesis of Amphiphiles, Peptide Antigen Conjugates, Drug Molecule Conjugates and any Precursors Thereof

Hydrophobic blocks (H) based on poly(amino acids) produced by solid phase peptide synthesis (SPPS) provide the advantage over hydrophobic polymers produced by radical polymerization that the resulting material obtained is chemically defined, i.e. a single product with an exact composition can be obtained.

However, a potential limitation of producing hydrophobic poly(amino acids) by SPPS is that highly hydrophobic peptides may not be soluble in the solvents commonly used for peptide coupling (e.g., DMF) and/or the hydrophobic peptides may not be suitable for purification using common HPLC mobile (e.g., acetonitrile and water) and stationary (e.g., C18) phases.

Therefore, we investigated the suitability of different hydrophobic poly(amino acids) based on amino acids having an aliphatic (Aliph), aryl (Ar), heteroaryl (H-Ar) or aminoaryl/aminoheteroaryl (Ar-a) side chain for synthesis by SPPS and purification by HPLC (Table 1).

TABLE 1
hydrophobic poly(amino acids)

| Cmpd # | Amino acid sequence | SEQ ID NO: | Length | Series | Synthesized | Purity |
|---|---|---|---|---|---|---|
| 1 | LLLLL | 52 | 5 | Aliph | Y | Crude |
| 2 | LLLLLLLLLL | 53 | 10 | Aliph | N | — |
| 3 | FFFFF | 54 | 5 | Ar | Y | 98% |
| 4 | FFFFFFFFFF | 55 | 10 | Ar | N | — |
| 5 | HHHHH | 56 | 5 | H—Ar | Y | Crude |
| 6 | HHHHHHHHHH | 57 | 10 | H—Ar | Y | Crude |
| 7 | WWWWW | 58 | 5 | H—Ar | Y | >95% |
| 8 | WWWWWWWWWW | 59 | 10 | H—Ar | Y | Crude |
| 9 | F′F′F′F′F′ | 60 | 5 | Ar-a | Y | >95% |
| 10 | F′F′F′F′F′F′F′F′F′F′ | 61 | 10 | Ar-a | Y | >95% |
| 11 | F′F′F′F′F′F′F′F′F′F′F′F′F′F′F′F′F′F′F′F′ | 62 | 20 | Ar-a | Y | >90% |
| 12 | F′F′F′F′F′F′F′F′F′F′F′F′F′F′F′F′F′F′F′F′F′ | 63 | 30 | Ar-a | Y | >90% |
| | F′F′F′F′F′F′F′F′F′ | | | | | |
| 13 | FWFWFWFWFW | 64 | 10 | H—Ar | N | — |
| 14 | WF′WF′WF′WF′WF′WF′WF′WF′WF′WF′ | 65 | 20 | H/Ar-a | Y | >90% |

L = leucine; F = phenylalanine; H = histidine; W = tryptophan; F′ = para-aminophenylalanine; Aliph = aliphatic-based poly(amino acid); Ar = aryl poly(amino acid); H—Ar = heteroaryl poly(amino acid); Ar-a = aminoaryl/aminoheteroaryl poly(amino acid); Y indicates successful synthesis upon first attempt; N indicates that the synthesis or purification of the specific amino acid sequence was not successful on the first attempt. Purity is the % AUC of the product determined by HPLC. Crude purity indicates that HPLC purification was not successful but crude material comprising the designated poly(amino acid)-based hydrophobic block (H) was obtained.

Our results show that hydrophobic poly(amino acids) comprising amino acids with Ar-a side chains were the most readily accessible by SPPS, followed by poly(amino acids) H-Ar, Ar and Aliph. Notably, poly(amino acids) comprising aliphatic side chains posed the greatest challenges to production by SPPS, while use of amino acids comprising aromatic side chains were generally more accessible, with poly(amino acids) comprising heteroaryl and aminoaryl/aminoheteroaryl groups being the most readily accessed.

These results indicate that aminoaryl/aminoheteroaryl improve synthesis and solubility of poly(amino acid)-based hydrophobic blocks (H) during both purification by HPLC as well as for improved solubility in water miscible solvents.

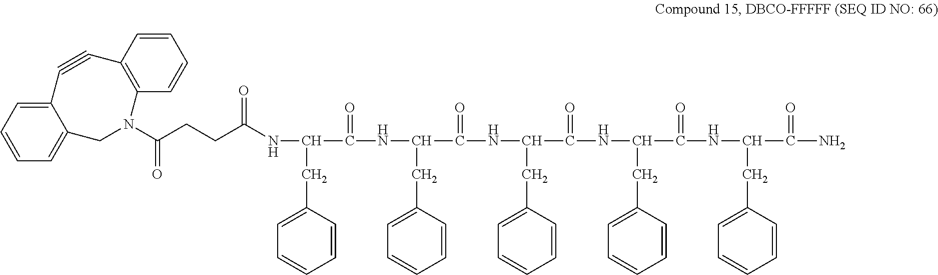 [SMILES: NC(=O)C(Cc1ccccc1)NC(=O)C(Cc1ccccc1)NC(=O)C(Cc1ccccc1)NC(=O)C(Cc1ccccc1)NC(=O)C(Cc1ccccc1)NC(=O)CCC(=O)N1Cc2ccccc2C#Cc2ccccc21]

Compound 15, referred to as DBCO-F₅, F₅ or DBCO-(Phe)₅ was synthesized by reacting 50.0 mg (0.066 mmol, 1 eq) of the precursor NH₂-(Phe)s-NH₂ that was prepared by solid phase peptide synthesis with 29.4 mg of DBCO-NHS (0.073 mmol, 1.1 eq) and 7.4 mg of triethylamine (0.073 mmol, 1.1 eq) in 1.0 mL of DMSO. Compound 15 was purified on a preparatory HPLC system using a gradient of 30-95% acetonitrile/H₂O (0.05% TFA) over 12 minutes on an Agilent Prep-C18 column, 50×100 mm, 5 μm. The product eluted at ˜10 minutes and the resulting fractions were collected, frozen and then lyophilized to obtain a spectroscopically pure (>95% AUC at 254 nm) white powder. MS (ESI) calculated for C₆₄H₆₁N₇O₇ m/z 1039.46, found 1040.6 (M+H)⁺.

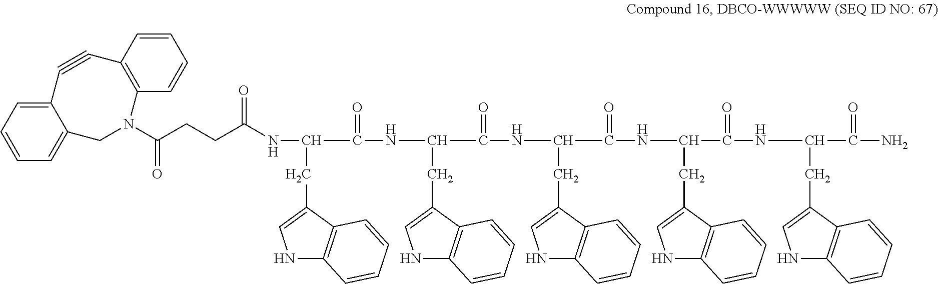 [SMILES: NC(=O)C(Cc1c[nH]c2ccccc12)NC(=O)C(Cc1c[nH]c2ccccc12)NC(=O)C(Cc1c[nH]c2ccccc12)NC(=O)C(Cc1c[nH]c2ccccc12)NC(=O)C(Cc1c[nH]c2ccccc12)NC(=O)CCC(=O)N1Cc2ccccc2C#Cc2ccccc21]

Compound 16, referred to as DBCO-W₅, W₅ or DBCO-(Trp)₅ was synthesized by reacting 137.6 mg (0.15 mmol, 1 eq) of the precursor NH₂-(Trp)s-NH₂ that was prepared by solid phase peptide synthesis with 146.1 mg of DBCO-NHS (0.057 mmol, 2.5 eq) and 14.7 mg of triethylamine (0.15 mmol, 1.1 eq) in 3.0 mL of DMSO. Compound 16 was purified on a preparatory HPLC system using a gradient of 52-72% acetonitrile/H₂O (0.05% TFA) over 12 minutes on an Agilent Prep-C18 column, 50×100 mm, 5 μm. The product eluted at ˜10 minutes and the resulting fractions were collected, frozen and then lyophilized to obtain 75.1 mg (42% yield) of a spectroscopically pure (>95% AUC at 254 nm) white powder. MS (ESI) calculated for C₇₄H₆₆N₁₂O₇ m/z 1234.52, found 1235.6 (M+H)⁺.

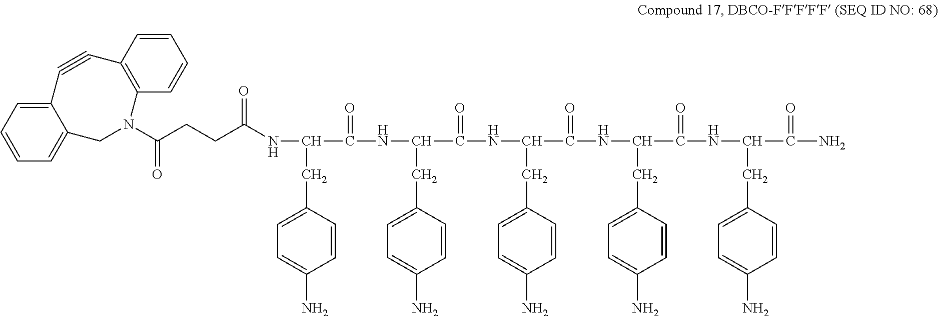 [SMILES: NC(=O)C(Cc1ccc(N)cc1)NC(=O)C(Cc1ccc(N)cc1)NC(=O)C(Cc1ccc(N)cc1)NC(=O)C(Cc1ccc(N)cc1)NC(=O)C(Cc1ccc(N)cc1)NC(=O)CCC(=O)N1Cc2ccccc2C#Cc2ccccc21]

Compound 17, referred to as DBCO-F′₅ or F′₅ was synthesized by reacting 49.8 mg (0.06 mmol, 1 eq) of the precursor NH₂-(F′)₅—NH₂, which was prepared by solid phase peptide synthesis, with 24.5 mg of DBCO-TT (0.057 mmol, 1.0 eq) and 30.3 mg of NaHCO₃ (0.36 mmol, 6.0 eq) in 1.0 mL of DMF. The reaction was run overnight at room temperature and HPLC indicated that the reaction was complete by 24 hours. Compound 17 was purified on a preparatory HPLC system using a gradient of 10-30% acetonitrile/H₂O (0.05% TFA) over 10 minutes on an Agilent Prep-C18 column, 30×100 mm, 5 gm. The product eluted at ˜3.4 minutes and the resulting fractions were collected, frozen and then lyophilized to obtain 25.8 mg (38.4% yield) of a spectroscopically pure (>95% AUC at 254 nm) white powder. MS (ESI) calculated for C₆₄H₆₆N₁₂O₇ m/z 1114.52, found 1116.1 (M+H)⁺.

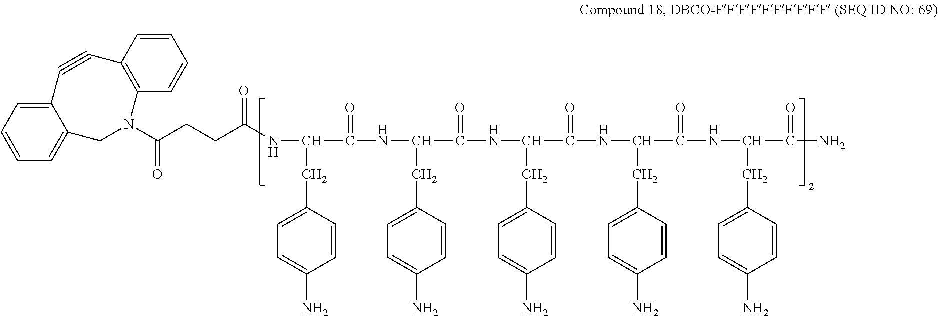

Compound 18, referred to as DBCO-F′₁₀ or F′₁₀ was synthesized by reacting 30 mg (0.0183 mmol, 1 eq) of the precursor NH₂-(F′)₁₀—NH₂, which was prepared by solid phase peptide synthesis, with 7.4 mg of DBCO-TT (0.018 mmol, 1.0 eq) and 16.9 mg of NaHCO₃ (0.20 mmol, 11 eq) in 1.0 mL of DMF. The reaction was run overnight at room temperature and HPLC indicated that the reaction was complete by 24 hours. Compound 18 was purified on a preparatory HPLC system using a gradient of 10-30% acetonitrile/H₂O (0.05% TFA) over 10 minutes on an Agilent Prep-C18 column, 30×100 mm, 5 gm. The product eluted at ˜6.3 minutes and the resulting fractions were collected, frozen and then lyophilized to obtain 14 mg (39.5% yield) of a spectroscopically pure (>95% AUC at 254 nm) white powder. MS (ESI) calculated for C₁₀₉H₁₁₆N₁₂O₁₂ m/z 1924.91, found 963.9 (M+2H)⁺.

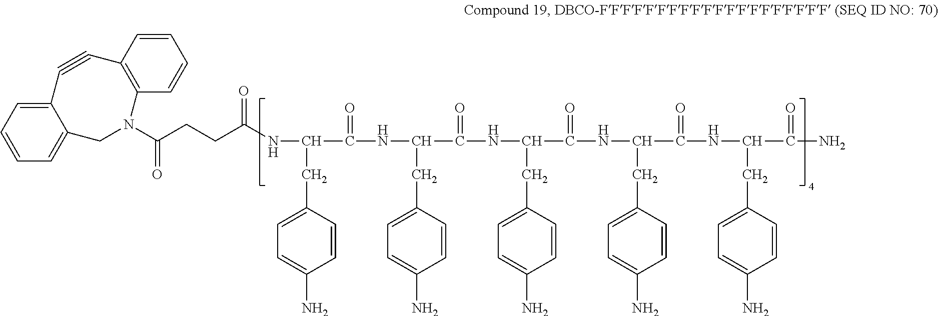

Compound 19, referred to as DBCO-F′₂₀ or F′₂₀ was synthesized by reacting 30 mg (0.009 mmol, 1 eq) of the precursor NH₂-(F′)₂₀—NH₂, which was prepared by solid phase peptide synthesis, with 3.7 mg of DBCO-TT (0.009 mmol, 1.0 eq) and 16.2 mg of NaHCO₃ (0.20 mmol, 21 eq) in 1.0 mL of DMF. The reaction was run overnight at room temperature and HPLC indicated that the reaction was complete by 24 hours. Compound 19 was purified on a preparatory HPLC system using a gradient of 10-30% acetonitrile/H₂O (0.05% TFA) over 10 minutes on an Agilent Prep-C18 column, 30×100 mm, 5 μm. The product eluted at ˜6.3 minutes and the resulting fractions were collected, frozen and then lyophilized to obtain 10.6 mg (32.4% yield) of a spectroscopically pure (>95% AUC at 254 nm) white powder. MS (ESI) calculated for C₁₉₉H₂₁₆N₄₂O₁₂ m/z 3545.71 found 1183.6 (M+3H)⁺ and 887 (M+4H)⁺.

As a facile process for producing amphiphiles of formula S-[B]-H, azide functionalized S-[B] were reacted with DBCO functionalized hydrophobic blocks (H). As an example, 0.5 mg of compound 15 in DMSO at 20 mg/mL was reacted with 1.0 mole equivalents of the peptide, KKSLVRX (SEQ ID NO:71), where X=azidolysine, which resulted in the complete conversion of starting material to compound 20:

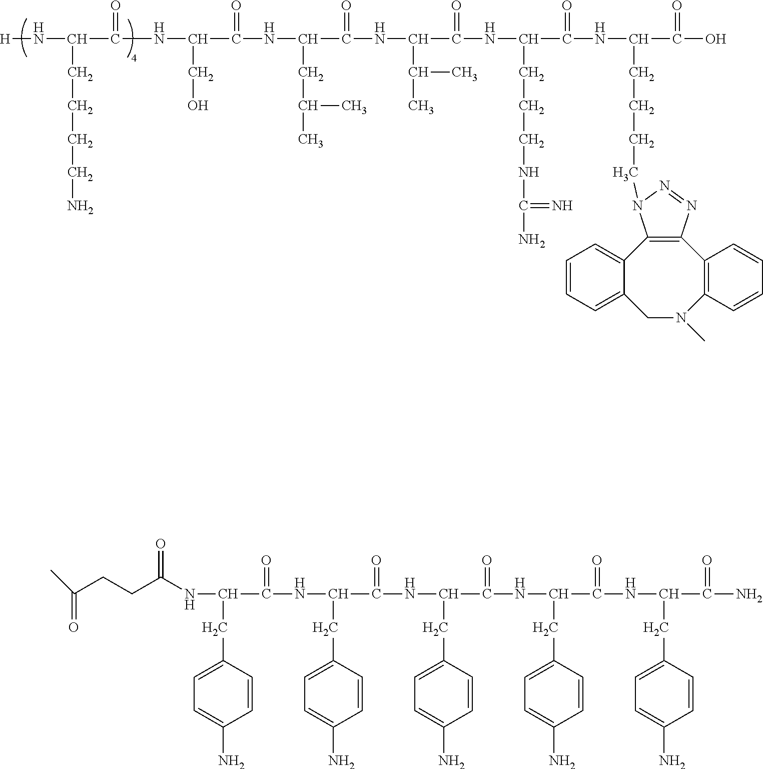

Similar reaction conditions were used to produce compounds 20-32 summarized in Table 2. Notable findings were that (i) amphiphilic block copolymers comprising aminoaryl and/or aminoheteroaryl typically resulted in stable micelles (˜20 nm size) without resulting in visible aggregates (i.e. turbidity>0.05 at 490 nm) or supramolecular associates (i.e. particle sizes>30 nm) and that (ii) net charge of the amphiphile greater than +4 or less than −4 were critical as amphiphiles comprising a short, neutral linear PEG as the solubilizing block (S) resulted in aggregates, which was not observed for amphiphiles bearing a solubilizing group (S) comprising charged amino acids.

TABLE 2
Hydrodynamic behavior of amphiphiles of formula S-[B]-U-H with linear
architecture

| | Sequence* | SEQ ID | | Aggregation | Particle size |
|---|---|---|---|---|---|
| # | S-B-(U)-H | NO: | MW | (OD > 0.05) | (Diameter, nm) |
| 20 | KKK-SLVRX-(N3-DBCO)-FFFFF | 72 | 2178.9 | AGGREGATE | — |
| 21 | EEEEE-(N3-DBCO)-FFFFF | 73 | 1828.19 | AGGREGATE | — |
| 22 | KKK-SLVRX-(N3-DBCO)-WWWWW | 74 | 2373.96 | SOLUBLE | 64.73 |
| 23 | EEEEE-(N3-DBCO)-WWWWW | 75 | 2023.25 | SOLUBLE | 54.09 |
| 24 | KKK-SLVRX-(N3-DBCO)-F′F′F′F′F′ | 76 | 2253.96 | SOLUBLE | 12.40 |
| 25 | EEEEE-(N3-DBCO)-F′F′F′F′F′ | 77 | 1903.25 | SOLUBLE | 14.79 |
| 26 | KKK-SLVRX-(N3-DBCO)- | 78 | 3064.35 | SOLUBLE | 15.71 |
| | F′F′F′F′F′F′F′F′F′F′ | | | | |
| 27 | EEEEE-(N3-DBCO)-F′F′F′F′F′F′F′F′ | 79 | 2713.64 | SOLUBLE | 17.46 |
| | F′F′ | | | | |
| 28 | OH-PEG24-(N3-DBCO)- | 80 | 3025.2 | AGGREGATE | — |
| | F′F′F′F′F′F′F′F′F′F′ | | | | |
| 29 | DDDDDDD-PEG24-(N3-DBCO)- | 81 | 4002.03 | SOLUBLE | 16.04 |
| | F′F′F′F′F′F′F′F′F′F′ | | | | |
| 30 | KKKKKKKKKKX-(N3-DBCO)- | 82 | 1452.93 | SOLUBLE | 8.41 |
| | F′F′F′F′F′F′F′F′F′F′ | | | | |
| 31 | KKK-SLVRX-(N3-DBCO)- | 83 | 4685.15 | SOLUBLE | 18.01 |
| | F′F′F′F′F′F′F′F′F′F′F′F′F′F′F′F′F′ | | | | |
| | F′F′F′ | | | | |
| 32 | EEEEE-(N3-DBCO)- | 84 | 4334.44 | AGGREGATE | — |
| | F′F′F′F′F′F′F′F′F′F′F′F′F′F′F′F′F′ | | | | |
| | F′F′F′ | | | | |

*Single letter code is used for the amine acid sequences listed in table 2; K = Lysine, S = Serine, L = leucine, V = valine, R = arginine, x = azidolysine, F = phenylalanine, W = tryptophan and F′ para-aminophenylalanine. (N3-DBCO) is the triazole linker that results from the reaction of an azide with DBCO. PEG24 refers to an ethylene oxide linker with 24 repeats.

An additional notable finding was that stable micelles could be formed by amphiphiles comprising hydrophobic blocks (H) with as few as 5 aromatic groups. Additional studies (data not shown) revealed, unexpectedly, that amphiphiles with hydrophobic blocks (H) with as few as 3 amino acids with aromatic side chains was sufficient to induce stable nanoparticle assembly; however, hydrophobic blocks (H) with between 5 to 30 amino acids were identified to be more preferable.

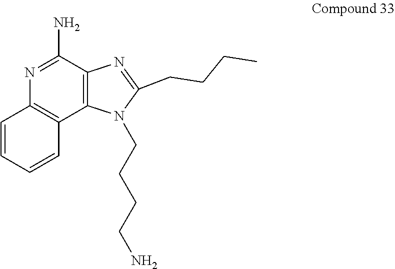 [SMILES: CCCCc1nc2c(N)nc3ccccc3c2n1CCCCN]

Compound 33, sometimes referred to as “2B,” 1-(4-aminobutyl)-2-butyl-1H-imidazo[4,5-c]quinolin-4-amine, referred to as 2B, was synthesized starting from 3-nitro-2,4-dichloroquinoline, 33-b, which was prepared as previously described (Lynn G M, et al., Nat Biotechnol 33(11):1201-1210, 2015). To 21 g of 33-b (87.8 mmol, 1 eq) in 210 mL of triethylamine (TEA) (10% w/w) was added 16.34 g (87.8 mmol, 1 eq) of N-boc-1,4-butanediamine while stirring vigorously. The reaction mixture was heated to 70° C. and monitored by HPLC, which confirmed that the reaction was complete after 2 hours. The triethylamine was removed under vacuum and the resulting oil was dissolved in 200 mL of dichloromethane and then washed with 3×100 mL DI H₂O. The organic layer was dried with Na₂SO₄ and then removed under vacuum and the resulting oil was triturated with 1:1 (v:v) hexane and diethyl ether to yield 30.7 g of yellow crystals of intermediate 33-c. MS (APCI) calculated for C₁₈H₂₃ClN₄O₄, m/z 394.1 found, 394.9.

33-d. 30.7 g (76.4 mmol) of intermediate 33-c was dissolved in 300 mL of ethyl acetate in a Parr Reactor vessel that was bubbled with argon, followed by the addition of 3 g of 10% platinum on carbon. The reaction vessel was kept under argon and then evacuated and pressurized with H₂(g) several times before pressurizing to 55 PSI H₂(g) while shaking vigorously. The H₂(g) was continually added until the pressure stabilized at 55 PSI, at which point the reaction was determined to be complete. The reaction mixture from the Parr Reactor was then filtered through celite end evaporated to dryness to obtain a yellow oil that was triturated with 1:1 hexanes/ether to yield white crystals that were collected by filtration to obtain 27.4 g of spectroscopically pure white crystals of 33-d. MS (APCI) calculated for C₁₈H₂₅ClN₄O₂, m/z 364.2, found 365.2.

33-e. To 10 g (27.4 mmol, 1 eq) of 33-d in 50 mL of THF was added 7.7 mL of triethylamine (54.8 mmol, 2 eq) followed by the drop wise addition of 3.6 g of valeroyl chloride (30.1 mmol, 1.1 eq) in 30 mL of THF while stirring vigorously while the reaction mixture was on ice. After 90 minutes, the ice bath was removed and the THF was removed under vacuum, resulting in a yellow oil that was dissolved in 100 mL of dichloromethane (DCM) that was washed with 3×50 mL of pH 5.5 100 mM acetate buffer. The DCM was removed under vacuum in an oil that was triturated with ethyl acetate to obtain 10.4 g of a white solid that was dissolved in methanol with 1 g of CaO (s), which was heated at 100° C. for 5 hours while stirring vigorously. The reaction mixture was filtered and dried to yield 10.2 g of an off-white solid, intermediate, 33-e. MS (ESI) calculated for C₂₃H₃₁C₁N₄02, m/z 430.21, found 431.2.

33-f. To 10.2 g (23.7 mmol, 1 eq) of 1-e was added 30.4 g (284 mmol, 12 eq) of benzylamine liquid, which was heated to 110° C. while stirring vigorously. The reaction was complete after 10 hours and the reaction mixture was added to 200 mL ethyl acetate and washed 4×100 mL with 1 M HCl. The organic layer was dried with Na₂SO₄ and then removed under vacuum and the resulting oil was recrystallized from ethyl acetate to obtain 10.8 g of spectroscopically pure white crystals of intermediate, 33-f. MS (ESI) calculated for C₃₀H₃₉N₅O₂, m/z 501.31, found 502.3

Compound 33. 10.8 g (21.5 mmol) of 33-f was dissolved in 54 mL of concentrated (>98%) H₂SO₄ and the reaction mixture was stirred vigorously for 3 hours. After 3 hours, viscous red reaction mixture was slowly added to 500 mL of DI H₂O while stirring vigorously. The reaction mixture was stirred for 30 minutes and then filtered through Celite, followed by the addition of 10 M NaOH until the pH of the solution was ˜pH 10. The aqueous layer was then extracted with 6×200 mL of DCM and the resulting organic layer was dried with Na₂SO₄ and reduced under vacuum to yield a spectroscopically pure white solid. ¹H NMR (400 MHZ, DMSO-d6) δ 8.03 (d, J=8.1 HZ, 1H), 7.59 (d, J=8.1 Hz, 1H), 7.41 (t, J=7.41 Hz, 1H), 7.25 (t, J=7.4 Hz, 1H), 6.47 (s, 2H), 4.49 (t, J=7.4 Hz, 2H), 2.91 (t, J=7.78 Hz, 2H), 2.57 (t, J=6.64 Hz, 1H), 1.80 (m, 4H), 1.46 (sep, J=7.75 Hz, 4H), 0.96 (t, J=7.4 Hz, 3H). MS (ESI) calculated for C₁₈H₂₅N₅, m/z 311.21, found 312.3.

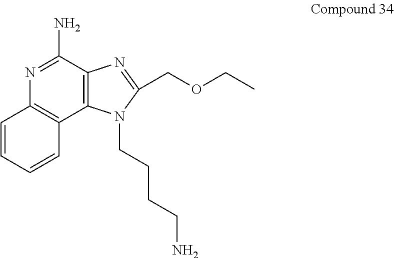 [SMILES: CCOCc1nc2c(N)nc3ccccc3c2n1CCCCN]

Compound 34 was prepared as previously described (Lynn G M, et al., Nat Biotechnol 33(11):1201-1210, 2015). ¹H NMR (400 MHz, DMSO-d6) δ 8.02 (dd, J=16.6, 8.2 Hz, 1H), 7.63-7.56 (m, 1H), 7.47-7.38 (m, 1H), 7.30-7.21 (m, 1H), 6.55 (s, 2H), 4.76 (s, 2H), 4.54 (q, J=6.3, 4.4 Hz, 2H), 3.54 (q, J=7.0 Hz, 2H), 2.58 (t, J=6.9 Hz, 2H), 1.93-1.81 (m, 2H), 1.52 (m, 2H), 1.15 (t, J=7.0 Hz, 3H). MS (APCI) calculated for C₁₇H₂₃N₅O m/z 313.2, found 314.2 (M+H)⁺.

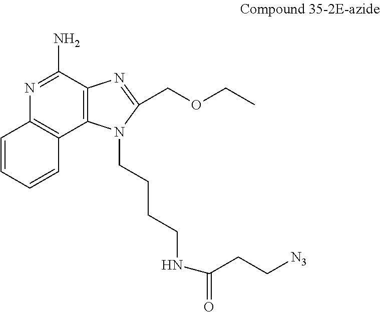 [SMILES: CCOCc1nc2c(N)nc3ccccc3c2n1CCCCNC(=O)CCN=[N+]=[N-]]

Compound 35 was prepared as previously described (Lynn G M, et al., Nat Biotechnol 33(11):1201-1210, 2015). MS (APCI) calculated for C₂₀H₂₆N₈O₂ m/z 410.2, found 411.2 (M+H)⁺.

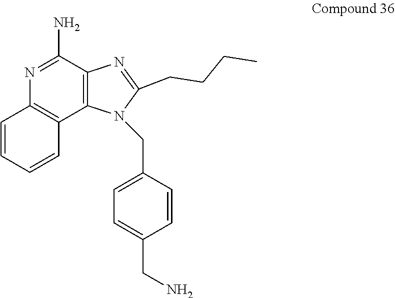 [SMILES: CCCCc1nc2c(N)nc3ccccc3c2n1Cc1ccc(CN)cc1]

Compound 36, 1-(4-(aminomethyl)benzyl)-2-butyl-1H-imidazo[4,5-c]quinolin-4-amine, referred to as 2BXy, was previously described (see: Lynn G M, et al., In vivo characterization of the physicochemical properties of polymer-linked TLR agonists that enhance vaccine immunogenicity. Nat Biotechnol 33(11):1201-1210, 2015, and Shukla N M, et al. Syntheses of fluorescent imidazoquinoline conjugates as probes of Toll-like receptor 7. Bioorg Med Chem Lett 20(22):6384-6386, 2010). 1H NMR (400 MHz, DMSO-d6) δ 7.77 (dd, J=8.4, 1.4 Hz, 1H), 7.55 (dd, J=8.4, 1.2 Hz, 1H), 7.35-7.28 (m, 1H), 7.25 (d, J=7.9 Hz, 2H), 7.06-6.98 (m, 1H), 6.94 (d, J=7.9 Hz, 2H), 6.50 (s, 2H), 5.81 (s, 2H), 3.64 (s, 2H), 2.92-2.84 (m, 2H), 2.15 (s, 2H), 1.71 (q, J=7.5 Hz, 2H), 1.36 (q, J=7.4 Hz, 2H), 0.85 (t, J=7.4 Hz, 3H). MS (APCI) calculated for C₂₂H₂₅N₅ m/z 359.2, found 360.3

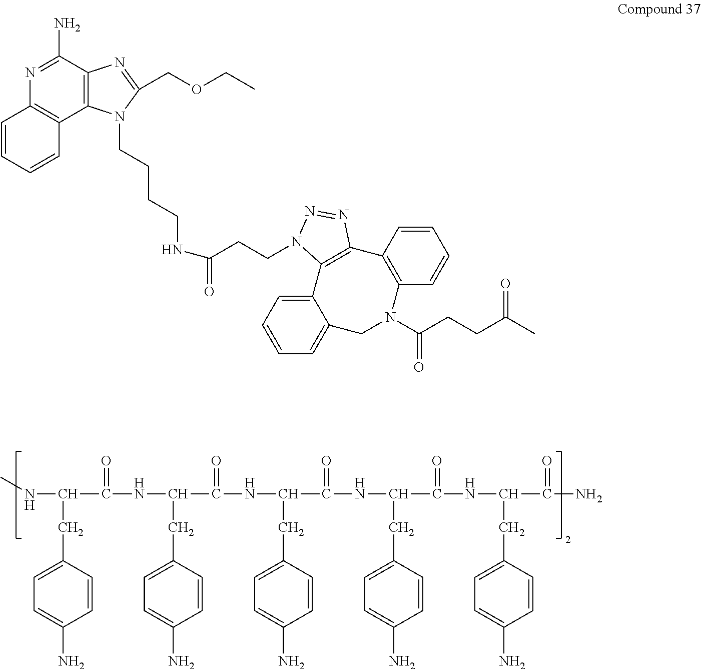

Compound 37 was produced by reacting 0.5 mg of compound 15 in DMSO at 20 mg/mL with 1.0 mole equivalents of compound 35, which resulted in the complete conversion of starting material to compound 37.

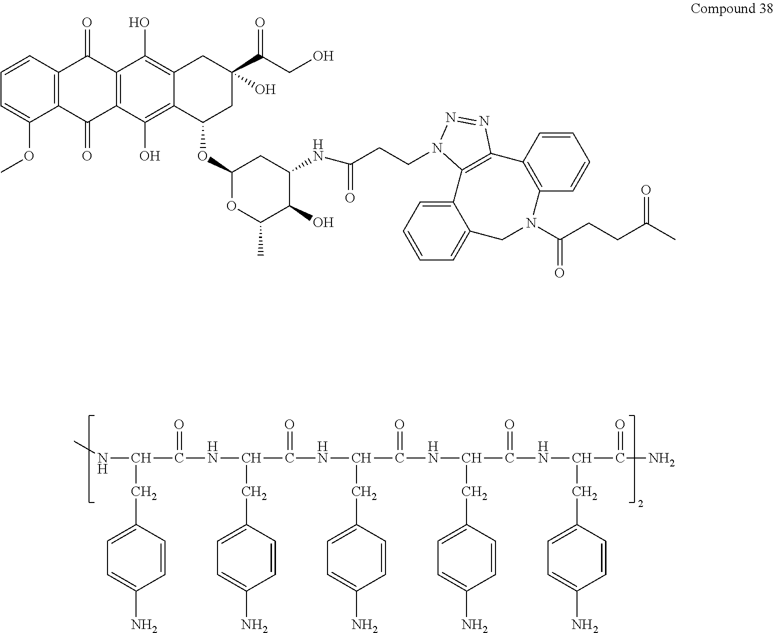

Compound 38 was produced by reacting 0.5 mg of compound 15 in DMSO at 20 mg/mL with 1.0 mole equivalents of azide functionalized doxorubicin, which resulted in the complete conversion of starting material to compound 38.

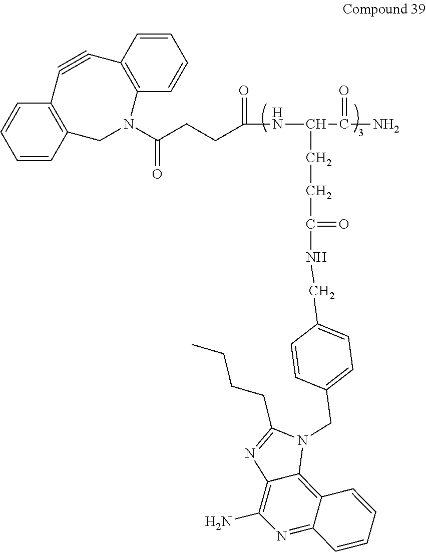

Compound 39, referred to as DBCO-2BXy₃, 2BXy₃ or DBCO-(Glu(2BXy)₃), was synthesized starting from an Fmoc-(Glu)₃-NH₂ precursor prepared by solid-phase peptide synthesis. 50 mg of Fmoc-(Glu)₃-NH₂ (0.08 mmol, 1 eq), 143 mg of Compound 36 (0.40 mmol, 5 eq), 84 mg of 2-chloro-4,6-dimethoxy-1,3,5-triazine (CDMT) (0.48 mmol, 6 eq) and 48.5 mg of 4-methylmorpholine (NMM) (0.48 mmol, 6 eq) were added to 3.25 mL of DMSO while stirring vigorously at room temperature under ambient air. The reaction progress was monitored by HPLC (AUC 254 nm). 1 additional equivalent of Compound 36 and 2 additional equivalents of both CDMT and NMM were added after 30 minutes. After 2 hours, the reaction was complete and the reaction mixture was added to 50 mL of a 1M HCl solution to precipitate the Fmoc protected intermediate, which was collected by centrifuging the solution at 3000 g at 4° C. for 10 minutes. The HCl solution was discarded and the Fmoc protected intermediate was collected as a solid white pellet. The white solid was re-suspended in 50 mL of a 1M HCl solution and spun at 3000 g at 4° C. for 5 minutes; the 1 M HCl solution was discarded and the product was collected as a solid pellet. This process was repeated and then the solid was collected and dried under vacuum to yield 156.1 mg of the Fmoc protected intermediate in quantitative yield. The Fmoc protected product was then added to 1.5 mL of a 20% piperidine in DMF solution for 30 minutes at room temperature to yield the deprotected product that was then precipitated from 50 mL of ether and centrifuged at 3000 g at 4° C. for 30 minutes. The product was collected as a solid pellet and then washed twice more with ether, followed by drying under vacuum to yield 126.4 mg of the intermediate. 60 mg of the resulting intermediate, NH₂-(Glu-2BXy)₃-NH₂, (0.042 mmol, 1 eq) was then reacted with 18.6 mg (0.046 mmol, 1.1 eq) of DBCO-NHS ester (Scottsdale, Arizona, USA) and 8.5 uL of triethylamine (0.084 mmol, 2 eq) in 1 mL of DMSO for 6 hours at room temperature. The resulting product, Compound 39, was purified on a preparatory HPLC system using a gradient of 30-70% acetonitrile/H₂O (0.05% TFA) over 12 minutes on an Agilent Prep-C18 column, 50×100 mm, 5 μm. The product eluted at 7.0 minutes and the resulting fractions were collected, frozen and then lyophilized to obtain 40.12 mg (55.7% yield) of a spectroscopically pure (>95% AUC at 254 nm) white powder. MS (ESI) calculated for C₁₀₀H₁₀₆N₂₀O₈ m/z 1714.85, found 858.9 (M/2)⁺

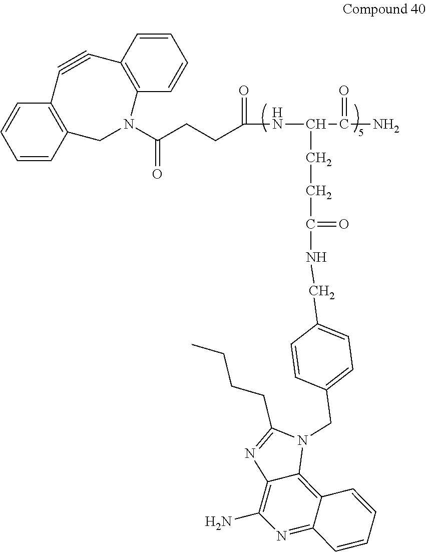

Compound 40, referred to as DBCO-2BXy₅, 2BXy₅ or DBCO-(Glu(2BXy)₅), was synthesized using the same procedure as described for Compound 39, except Fmoc-(Glu)s-NH₂ was used as the starting material for conjugation of Compound 36. Compound 40 was purified on a preparatory HPLC system using a gradient of 38-48% acetonitrile/H₂O (0.05% TFA) over 12 minutes on an Agilent Prep-C18 column, 50×100 mm, 5 μm. The product eluted at 8.0 minutes and the resulting fractions were collected, frozen and then lyophilized to obtain 45.9 mg (63.4% yield) of a spectroscopically pure (>95% AUC at 254 nm) white powder. MS (ESI) calculated for C₁₅₄H₁₆₆N₃₂O₁₂ m/z 2655.34, found 886.6 (M/3)⁺.

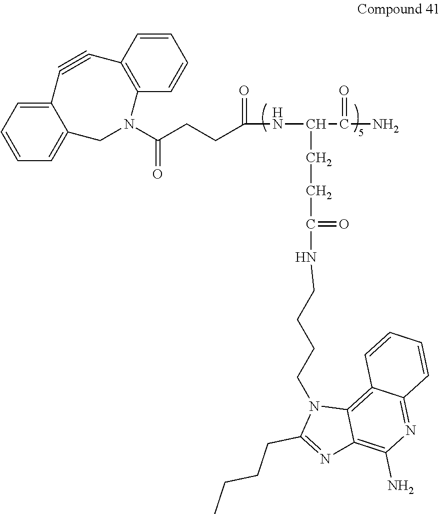

Compound 41, referred to as DBCO-2B₅, 2B₅ or DBCO-(Glu(2B)₅), was synthesized using the same procedure as described for Compound 39, except Fmoc-(Glu)s-NH₂ (SEQ ID NO:85), was used as the starting material for conjugation of Compound 33. Compound 41 was purified on a preparatory HPLC system using a gradient of 33-45% acetonitrile/H₂O (0.05% TFA) over 12 minutes on an Agilent Prep-C18 column, 50×100 mm, 5 μm. The product eluted at ˜10.0 minutes and the resulting fractions were collected, frozen and then lyophilized to obtain 25.2 mg (62.6% yield) of a spectroscopically pure (>95% AUC at 254 nm) white powder. MS (ESI) calculated for C₁₃₄H₁₆₆N₃₂O₁₂ m/z 2415.34, found 1209.3 (M/2)⁺.

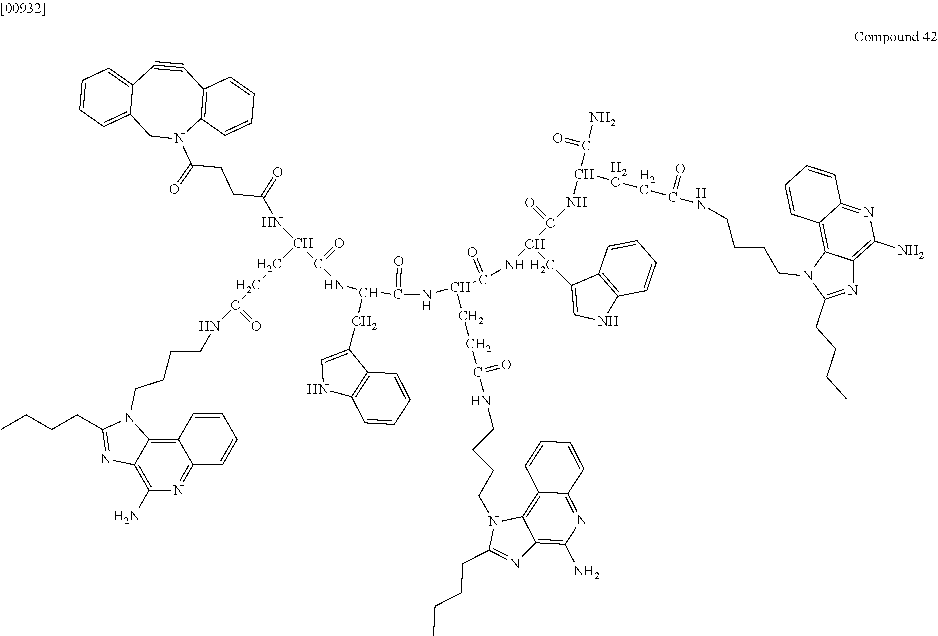 [SMILES: CCCCc1nc2c(N)nc3ccccc3c2n1CCCCNC(=O)CCC(NC(=O)C(Cc1c[nH]c2ccccc12)NC(=O)C(CCC(=O)NCCCCn1c(CCCC)nc2c(N)nc3ccccc3c21)NC(=O)C(Cc1c[nH]c2ccccc12)NC(=O)C(CCC(=O)NCCCCn1c(CCCC)nc2c(N)nc3ccccc3c21)NC(=O)CCC(=O)N1Cc2ccccc2C#Cc2ccccc21)C(N)=O]

Compound 42, referred to as DBCO-2B₃W2, 2B₃W₂ or DBCO-(Glu(2B)₃(Trp)₂), was synthesized using the same procedure as described for Compound 39, except Fmoc-Glu-Trp-Glu-Trp-Glu-NH₂ (SEQ ID NO:86), was used as the starting material for conjugation of Compound 33. Compound 42 was purified on a preparatory HPLC system using a gradient of 33-47% acetonitrile/H₂O (0.05% TFA) over 12 minutes on an Agilent Prep-C18 column, 50×100 mm, 5 μm. The product eluted at ˜8 minutes and the resulting fractions were collected, frozen and then lyophilized to obtain 197 mg (50.6% yield) of a spectroscopically pure (>95% AUC at 254 nm) white powder. MS (ESI) calculated for C₁₁₀H₁₂₆N₂₄O₁₀ m/z 1943.01, found 973.0 (M/2)⁺.

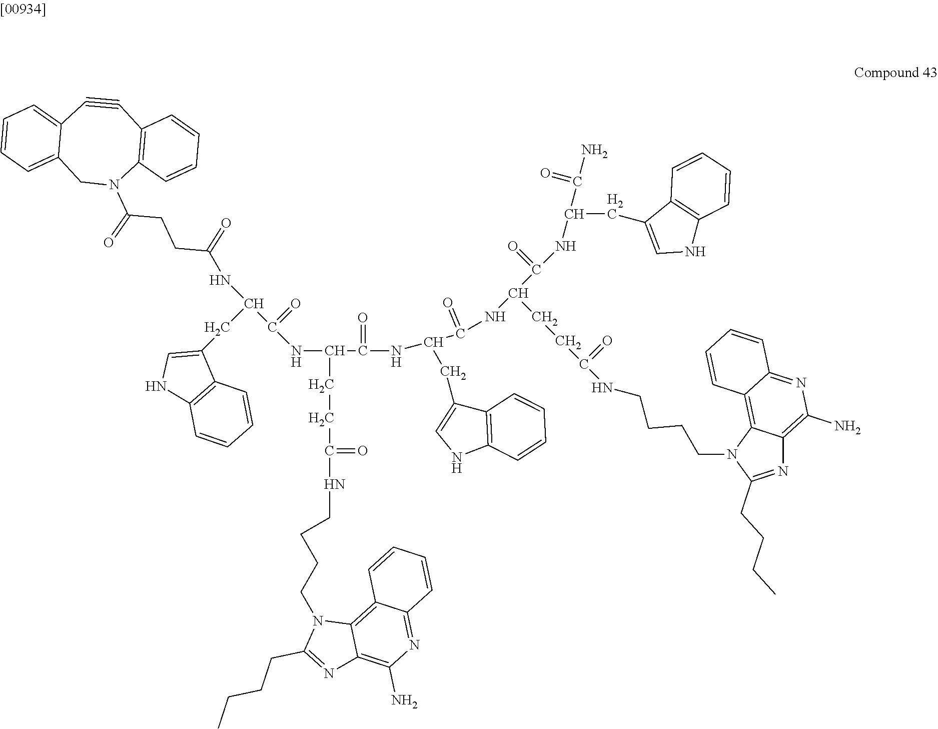 [SMILES: CCCCc1nc2c(N)nc3ccccc3c2n1CCCCNC(=O)CCC(NC(=O)C(Cc1c[nH]c2ccccc12)NC(=O)C(CCC(=O)NCCCCn1c(CCCC)nc2c(N)nc3ccccc3c21)NC(=O)C(Cc1c[nH]c2ccccc12)NC(=O)CCC(=O)N1Cc2ccccc2C#Cc2ccccc21)C(=O)NC(Cc1c[nH]c2ccccc12)C(N)=O]

Compound 43, referred to as DBCO-2B₂W3, 2B₂W₃ or DBCO-(Glu(2B)₂(Trp)₃), was synthesized using the same procedure as described for Compound 39, except Fmoc-Trp-Glu-Trp-Glu-Trp-NH₂ (SEQ ID NO:87), was used as the starting material for conjugation of Compound 33. Compound 43 was purified on a preparatory HPLC system using a gradient of 35-65% acetonitrile/H₂O (0.05% TFA) over 12 minutes on an Agilent Prep-C18 column, 50×100 mm, 5 m. The product eluted at ˜9 minutes and the resulting fractions were collected, frozen and then lyophilized to obtain 11.6 mg (62.5% yield) of a spectroscopically pure (>95% AUC at 254 nm) white powder. MS (ESI) calculated for C₉₈H₁₀₆N₂₀O₉ m/z 1706.85, found 854.9 (M/2)⁺.

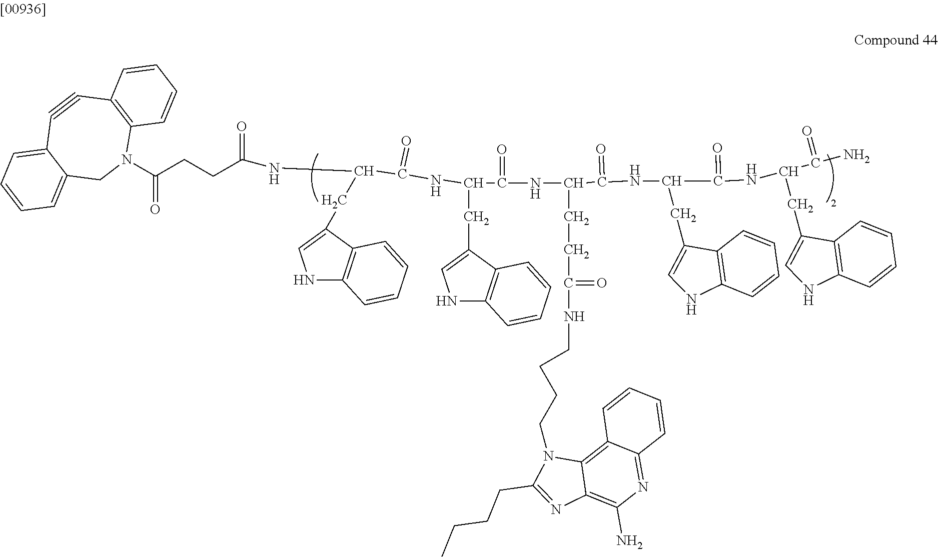

Compound 44, referred to as DBCO-2B₂W8, 2B₂W₈ or DBCO-(Glu(2B)₂(Trp)₈), was synthesized using the same procedure as described for Compound 39, except Fmoc-Trp-Trp-Glu-Trp-Trp-Trp-Trp-Glu-Trp-Trp-NH₂ (SEQ ID NO:88), was used as the starting material for conjugation of Compound 33. Compound 44 was purified on a preparatory HPLC system using a gradient of 35-85% acetonitrile/H₂O (0.05% TFA) over 12 minutes on an Agilent Prep-C18 column, 50×100 mm, 5 μm. The product eluted at ˜8.0 minutes and the resulting fractions were collected, frozen and then lyophilized to obtain 3.3 mg (16.3% yield) of a spectroscopically pure (>95% AUC at 254 nm) white powder. MS (ESI) calculated for C₁₅₃H₁₅₆N₃₀O₁₄ m/z 2637.24, found 1320.2 (M/2)⁺.

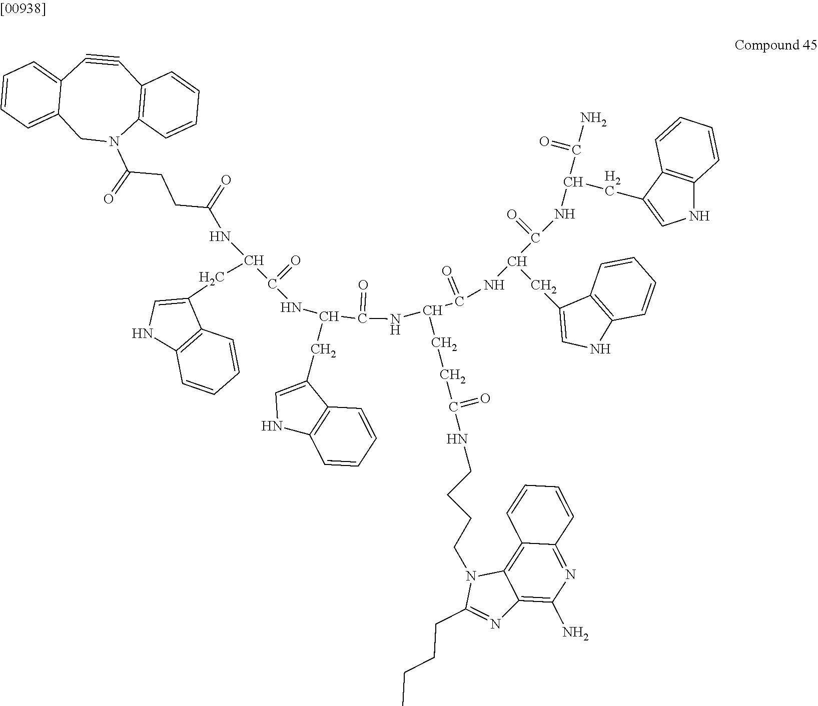 [SMILES: CCCCc1nc2c(N)nc3ccccc3c2n1CCCCNC(=O)CCC(NC(=O)C(Cc1c[nH]c2ccccc12)NC(=O)C(Cc1c[nH]c2ccccc12)NC(=O)CCC(=O)N1Cc2ccccc2C#Cc2ccccc21)C(=O)NC(Cc1c[nH]c2ccccc12)C(=O)NC(Cc1c[nH]c2ccccc12)C(N)=O]

Compound 45 referred to as DBCO-2B₁W4, 2B₁W₄ or DBCO-(Glu(2B)₁(Trp)₄), was synthesized using the same procedure as described for Compound 39, except Fmoc-Trp-Trp-Glu-Trp-Trp-NH₂ (SEQ ID NO:89), was used as the starting material for conjugation of Compound 33. Compound 45 was purified on a preparatory HPLC system using a gradient of 50-55% acetonitrile/H₂O (0.05% TFA) over 12 minutes on an Agilent Prep-C18 column, 50×100 mm, 5 μm. The product eluted at 8.9 minutes and the resulting fractions were collected, frozen and then lyophilized to obtain 9.7 mg (55.4% yield) of a spectroscopically pure (>95% AUC at 254 nm) white powder. MS (ESI) calculated for C₈₆H₈₆N₁₆O₈ m/z 1470.68, found 736.6 (M/2)⁺.

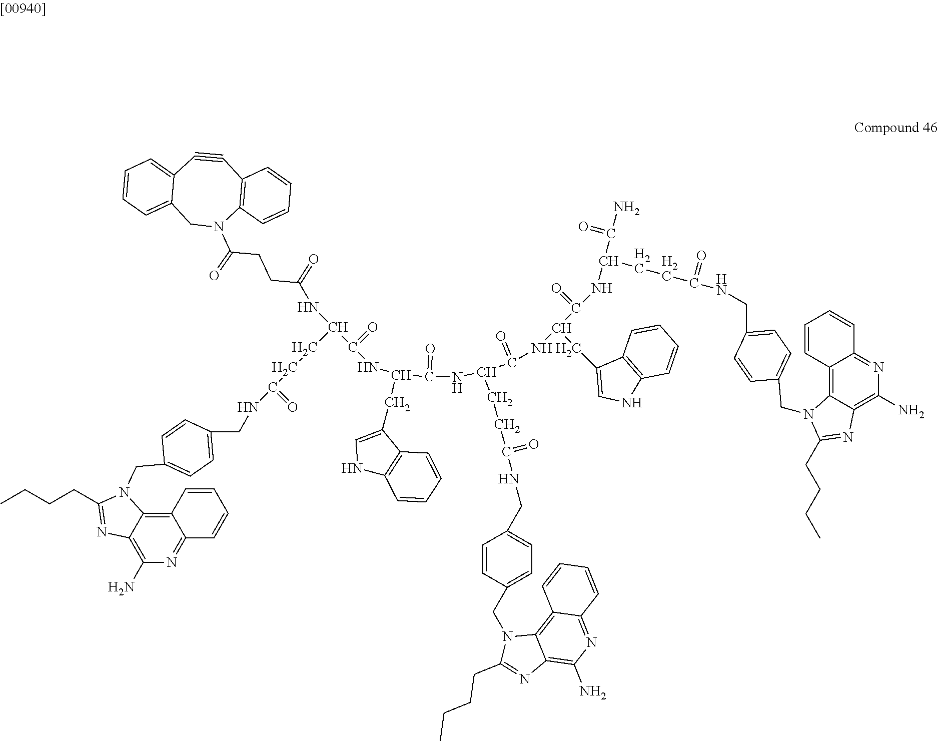 [SMILES: CCCCc1nc2c(N)nc3ccccc3c2n1Cc1ccc(CNC(=O)CCC(NC(=O)C(Cc2c[nH]c3ccccc23)NC(=O)C(CCC(=O)NCc2ccc(Cn3c(CCCC)nc4c(N)nc5ccccc5c43)cc2)NC(=O)C(Cc2c[nH]c3ccccc23)NC(=O)C(CCC(=O)NCc2ccc(Cn3c(CCCC)nc4c(N)nc5ccccc5c43)cc2)NC(=O)CCC(=O)N2Cc3ccccc3C#Cc3ccccc32)C(N)=O)cc1]

Compound 46, referred to as DBCO-2BXy₃W2, 2BXy₃W₂ or DBCO-(Glu(2BXy)₃(Trp)₂), was prepared using Fmoc-Glu-Trp-Glu-Trp-Glu-NH₂ (SEQ ID NO:90) and Compound 33 as the starting materials. 500 mg of Fmoc-Glu-Trp-Glu-Trp-Glu-NH₂ (SEQ ID NO:90), (0.5 mmol, 1 eq), 595.6 mg of Thiazoline-2-Thiol (TT) (5 mmol, 10 eq), and 575.7 mg of 1-Ethyl-3-(3-dimethylaminopropyl)carbodiimide (EDC) (3 mmol, 6 eq) were suspended in 26 mL of DCM. 18.3 mg of 4-(Dimethylamino)pyridine (DMAP) (0.2 mmol, 0.3 eq) was added and the reaction mixture was stirred at room temperature. The reaction progress was monitored by analytical HPLC. After 4 hours, an additional four equivalents of TT and two equivalents of EDC were added. After stirring overnight, two equivalents of TT and a half equivalent of EDC were added. After 6 hours, the reaction was complete. The DCM was removed under vacuum and the solid was taken up in 6 mL of dry DMSO. 539.3 mg of Compound 33 (1.5 mmol, 3 eq) was added and the reaction mixture was stirred for 2 hours at room temperature. The conjugated intermediate was then precipitated from 300 mL of 1 M HCl and centrifuged at 3000 g at 4° C. for 10 minutes. The pellet was collected and washed once more with 1 M HCl and once with DI water. The final collected pellet was frozen and dried under vacuum. 809.06 mg of Fmoc-2BXy₃W₂—NH₂ (0.4 mmol, 1 eq)) was dissolved in 4 mL of 20% piperidine in DMF. The reaction mixture was stirred at room temperature for 1 hour. The deprotected intermediate was then precipitated from 100 mL of ether and centrifuged at 3000 g at 4° C. for 10 minutes. The product was collected as a solid pellet and then washed twice more with ether, followed by drying under vacuum to yield the intermediate. 729 mg NH₂-2BXy₃W₂—NH₂ (0.4 mmol, 1 eq) was dissolved in 6 mL of dry DMSO. 488.8 mg of DBCO-NHS (1.2 mmol, 3 eq) was added and the reaction mixture was stirred at room temperature for 1 hour. The resulting product was purified on a preparatory HPLC system using a gradient of 36-46% acetonitrile/H2O (0.05% TFA) over 12 minutes on an Agilent Prep C-18 column, 50×100 mm, 5 μm. The resulting fractions were combined, frozen and lyophilized to give 239 mg (38.1% yield) of a spectroscopically pure of white powder. MS (ESI) Calculated for C₁₂₂H₁₂₆N₂₄O₁₀ m/z 2087.65 found 697 (m/3)⁺.

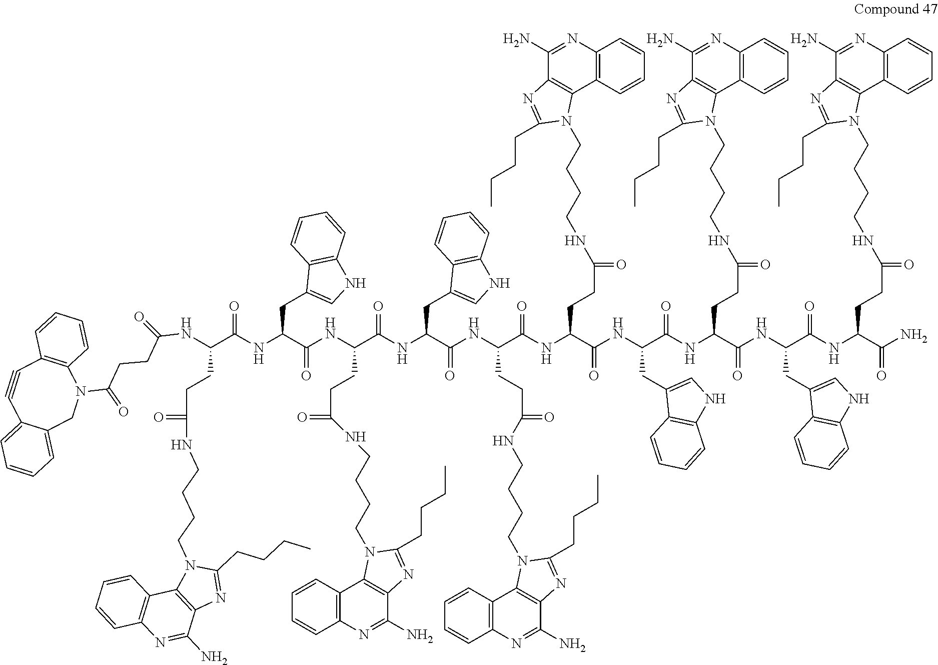 [SMILES: CCCCc1nc2c(N)nc3ccccc3c2n1CCCCNC(=O)CC[C@H](NC(=O)[C@H](Cc1c[nH]c2ccccc12)NC(=O)[C@H](CCC(=O)NCCCCn1c(CCCC)nc2c(N)nc3ccccc3c21)NC(=O)[C@H](Cc1c[nH]c2ccccc12)NC(=O)[C@H](CCC(=O)NCCCCn1c(CCCC)nc2c(N)nc3ccccc3c21)NC(=O)[C@H](CCC(=O)NCCCCn1c(CCCC)nc2c(N)nc3ccccc3c21)NC(=O)[C@H](Cc1c[nH]c2ccccc12)NC(=O)[C@H](CCC(=O)NCCCCn1c(CCCC)nc2c(N)nc3ccccc3c21)NC(=O)[C@H](Cc1c[nH]c2ccccc12)NC(=O)[C@H](CCC(=O)NCCCCn1c(CCCC)nc2c(N)nc3ccccc3c21)NC(=O)CCC(=O)N1Cc2ccccc2C#Cc2ccccc21)C(N)=O]

Compound 47, referred to as DBCO-2B₆W_{4, 2}B₆W₄ or DBCO-(Glu(2B)₆(Trp)₄), was synthesized using the same procedure as described for Compound 39, except Fmoc-(Glu-Trp-Glu-Trp-Glu)₂-NH₂ (SEQ ID NO:91), was used as the starting material for conjugation of Compound 33. Compound 47 was purified on a preparatory HPLC system using a gradient of 24-45% acetonitrile/H₂O (0.05% TFA) over 10 minutes on an Agilent Prep-C18 column, 30×100 mm, 5 μm. The fractions were collected, frozen and then lyophilized to obtain a spectroscopically pure (>95% AUC at 254 nm) white powder. MS (ESI) calculated for C₂₀₁H₂₃₆N₄₆O₁₈ m/z 3582.4, found 717.7 (M/5)+

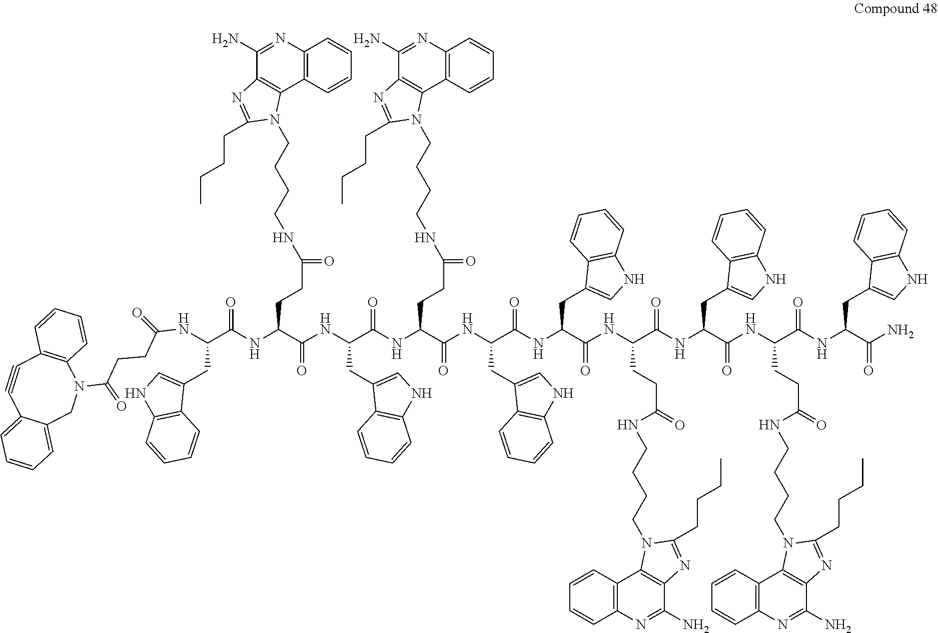 [SMILES: CCCCc1nc2c(N)nc3ccccc3c2n1CCCCNC(=O)CC[C@H](NC(=O)[C@H](Cc1c[nH]c2ccccc12)NC(=O)[C@H](CCC(=O)NCCCCn1c(CCCC)nc2c(N)nc3ccccc3c21)NC(=O)[C@H](Cc1c[nH]c2ccccc12)NC(=O)[C@H](Cc1c[nH]c2ccccc12)NC(=O)[C@H](CCC(=O)NCCCCn1c(CCCC)nc2c(N)nc3ccccc3c21)NC(=O)[C@H](Cc1c[nH]c2ccccc12)NC(=O)[C@H](CCC(=O)NCCCCn1c(CCCC)nc2c(N)nc3ccccc3c21)NC(=O)[C@H](Cc1c[nH]c2ccccc12)NC(=O)CCC(=O)N1Cc2ccccc2C#Cc2ccccc21)C(=O)N[C@@H](Cc1c[nH]c2ccccc12)C(N)=O]

Compound 48, referred to as DBCO-2B₄W_{6, 2}B₄W₆ or DBCO-(Glu(2B)₄(Trp)₆), was synthesized using the same procedure as described for Compound 39, except Fmoc-(Trp-Glu-Trp-Glu-Trp)₂-NH₂ (SEQ ID NO:92), was used as the starting material for conjugation of Compound 33. Compound 48 was purified on a preparatory HPLC system using a gradient of 24-45% acetonitrile/H₂O (0.05% TFA) over 10 minutes on an Agilent Prep-C18 column, 30×100 mm, 5 μm. The fractions were collected, frozen and then lyophilized to obtain a spectroscopically pure (>95% AUC at 254 nm) white powder. MS (ESI) calculated for C₁₇₇H₁₉₆N₃₈O₁₆ m/z 3111.7, found 777.5 (M/4)⁺.

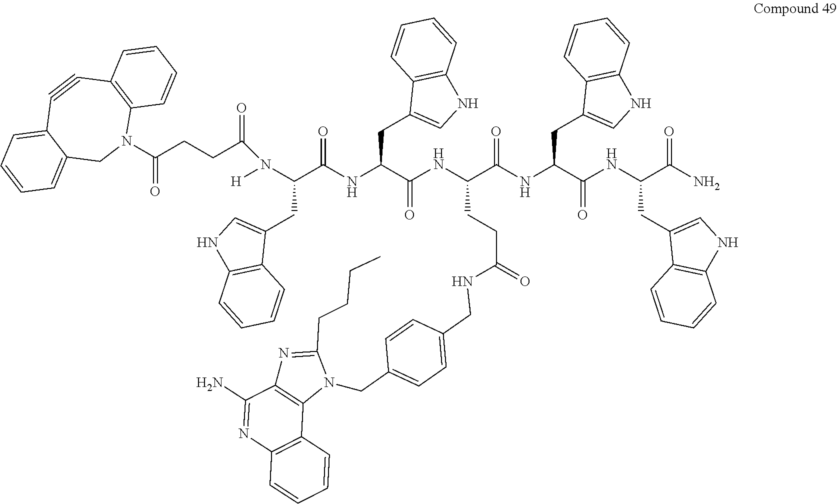 [SMILES: CCCCc1nc2c(N)nc3ccccc3c2n1Cc1ccc(CNC(=O)CC[C@H](NC(=O)[C@H](Cc2c[nH]c3ccccc23)NC(=O)[C@H](Cc2c[nH]c3ccccc23)NC(=O)CCC(=O)N2Cc3ccccc3C#Cc3ccccc32)C(=O)N[C@@H](Cc2c[nH]c3ccccc23)C(=O)N[C@@H](Cc2c[nH]c3ccccc23)C(N)=O)cc1]

Compound 49, referred to as DBCO-2BXy₁W_{4, 2}BXy₁W₄ or DBCO-(Glu(2BXy)₁(Trp)₄), was prepared using the same procedure as described for Compound 39, except Fmoc-Trp-Trp-Glu-Trp-Trp-NH₂ was used as the starting material. Compound 49 was purified on a preparatory HPLC system using a gradient of 40-70% acetonitrile/H₂O (0.05% TFA) over 16 minutes on an Agilent Prep-C18 column, 30×100 mm, 5 μm. The resulting fractions were collected, frozen and then lyophilized to obtain 3.4 mg (73.3% yield) of a spectroscopically pure (>95% AUC at 254 nm) white powder. MS (ESI) calculated for C₉₀H₈₅N₁₅O₉ m/z 1519.67, found 760.5 (M/2)⁺.

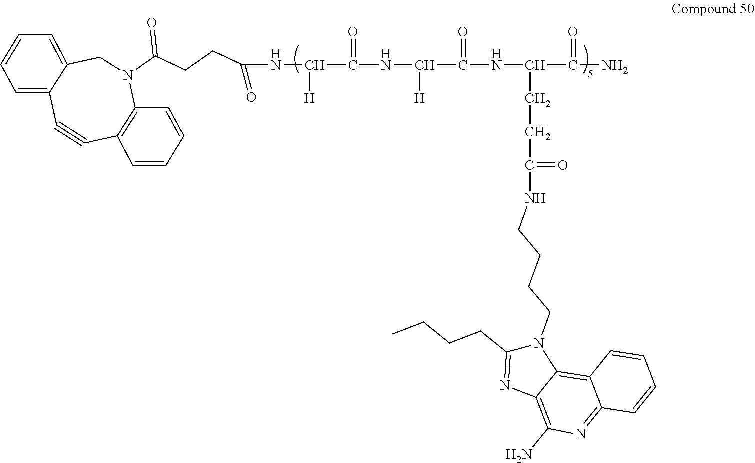

Compound 50, referred to as DBCO-(GG2B)₅, 2B₅G₁₀ or DBCO-(Glu(2B)₅(Gly)₁₀), was synthesized using the same procedure described for Compound 39, except Fmoc-(Gly-Gly-Glu)s-NH₂ and Compound 33 were used as the starting materials. Compound 50 was purified on a preparatory HPLC system using a gradient of 22-42% acetonitrile/H₂O (0.05% TFA) over 12 minutes on an Agilent Prep-C18 column, 30×100 mm, 5 μm. The product eluted at 7 minutes and the resulting fractions were collected, frozen and then lyophilized to obtain 22.8 mg (36.2% yield) of a spectroscopically pure (>95% AUC at 254 nm) white powder. MS (ESI) calculated C₁₅₄H₁₉₆N₄₂O₂₂ for m/z 2985.51, found 598.5 (M/5)⁺.

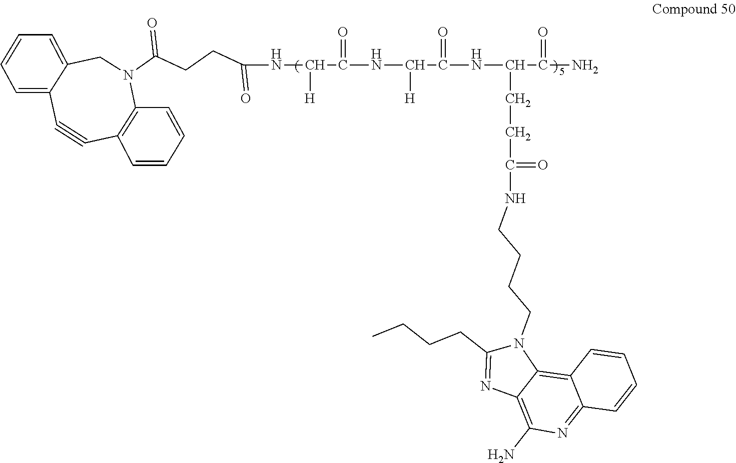

Compound 51, referred to as DBCO-(GG2BGGW)₂GG2B, 2B₃W₂G₁₀ or DBCO-(Glu(2B)₃(Trp)₂(Gly)₁₀), was synthesized from Fmoc-(Gly-Gly-Glu-Gly-Gly-Trp)₂-Gly₂-Glu-NH₂ (SEQ ID NO:93), precursor prepared by solid-phase peptide synthesis and Compound 33. 235.4 mg of Fmoc-(Gly-Gly-Glu-Gly-Gly-Trp)₂-Gly₂-Glu-NH₂ (SEQ ID NO:93), (0.15 mmol, 1 eq) was dissolved in 2 mL of 20% Piperidine in DMF. After 30 minutes the reaction was complete and the product was precipitated from 100 mL of ether and centrifuged at 3000 g at 4° C. for 10 minutes. The product was collected as a solid pellet and then washed twice more with ether, followed by drying under vacuum to yield˜200 mg of the deprotected intermediate. 200 mg (0.15 mmol, 1 eq) of NH₂-(Gly-Gly-Glu-Gly-Gly-Trp)₂-Gly₂-Glu-NH₂ (SEQ ID NO:94), was dissolved in 2 mL of dry DMSO and 89.73 mg of DBCO-NHS (0.22 mmol, 1.5 eq) was added followed by TEA (0.22 mmol, 1.5 eq). The reaction mixture was stirred at room temperature for 1 hour. The resulting DBCO intermediate was purified on a preparatory HPLC system using a gradient of 30-50% acetonitrile/H2O (0.05% TFA) over 12 minutes on an Agilent Prep C-18 column, 50×100 mm, 5 μm. The resulting fractions were combined, frozen and lyophilized to give the intermediate. 25 mg of DBCO-(Gly-Gly-Glu-Gly-Gly-Trp)₂-Gly₂-Glu-NH₂(SEQ ID NO:95), (0.015 mmol, 1 eq) and 17.11 mg of Compound 33 (0.055 mmol, 3.6 eq) were dissolved in 1.2 mL of dry DMSO. TEA (0.183 mmol, 12 eq) was added and the reaction mixture was stirred at room temperature for 5 minutes. 19.17 mg of HATU (0.05 mmol, 3.3 eq) was added and the reaction mixture was stirred at room temperature. The progress of the reaction was monitored by LC-MS. 1.2 additional equivalents of Compound 33 and 1.1 equivalents HATU were added after 1 hour. After 2 hours, the reaction was complete. The resulting product was purified on a preparatory HPLC system using a gradient of 30-60% acetonitrile/H2O (0.05% TFA) over 12 minutes on an Agilent Prep C-18 column, 30×100 mm, 5 μm. The resulting fractions were combined, frozen and lyophilized to give a spectroscopically pure white powder. MS (ESI) Calculated for C₁₃₀H₁₅₆N₃₄O₂₀ m/z 2515.96 found 839 (m/3)⁺.

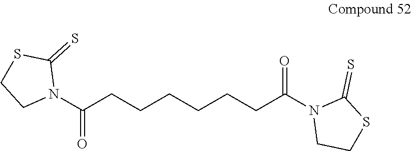 [SMILES: O=C(CCCCCCC(=O)N1CCSC1=S)N1CCSC1=S]

Compound 52, referred to as Bis(TT), was synthesized using Suberic acid and 2-thiazoline-2-thiol (TT) as starting materials. Briefly, 500 mg of Suberic acid (2.87 mmol, 1 eq), 752.7 mg of TT (6.31 mmol, 2.2 eq) and 1.431 g of EDC (7.46 mmol, 2.6 eq) were dissolved in 17.5 mL of dry DMSO. 70.15 mg of DMAP (0.57 mmol, 0.2 eq) was added and the reaction mixture was stirred at room temperature for 1 hour. The reaction mixture was diluted with DCM and washed twice with 1 M HCl and once with DI water. The organic fractions were dried with sodium sulfate and evaporated under reduced pressure to provide a yellow solid in quantitative yield.

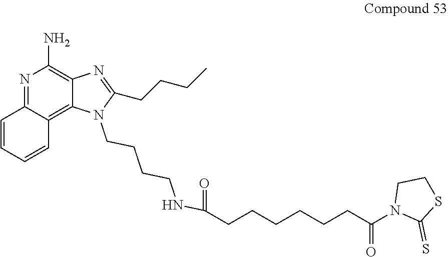 [SMILES: CCCCc1nc2c(N)nc3ccccc3c2n1CCCCNC(=O)CCCCCCC(=O)N1CCSC1=S]

Compound 53, referred to as 2B-TT, was synthesized using Compound 52 and Compound 33 as starting materials. Briefly, 50 mg (0.16 mmol, 1 eq) of Compound 33 was dissolved in 0.6 mL of methanol and added dropwise to a vigorously stirring solution of 301.1 mg of Compound 53 (0.8 mmol, 5 eq) in 1.93 mL of DCM. After 30 minutes, the reaction mixture was injected directly onto a column and purified by flash chromatography using a 2-step gradient: 5% methanol in DCM over 5 column volumes (CVs), followed by a 5-50% methanol in DCM gradient over 20 CVs. The fractions were combined and the solvent was removed under vacuum. MS (ESI) calculated for C₂₉H₄₀N₆O₂S₂ m/z 568.27 found 569.3 (m+H)⁺.

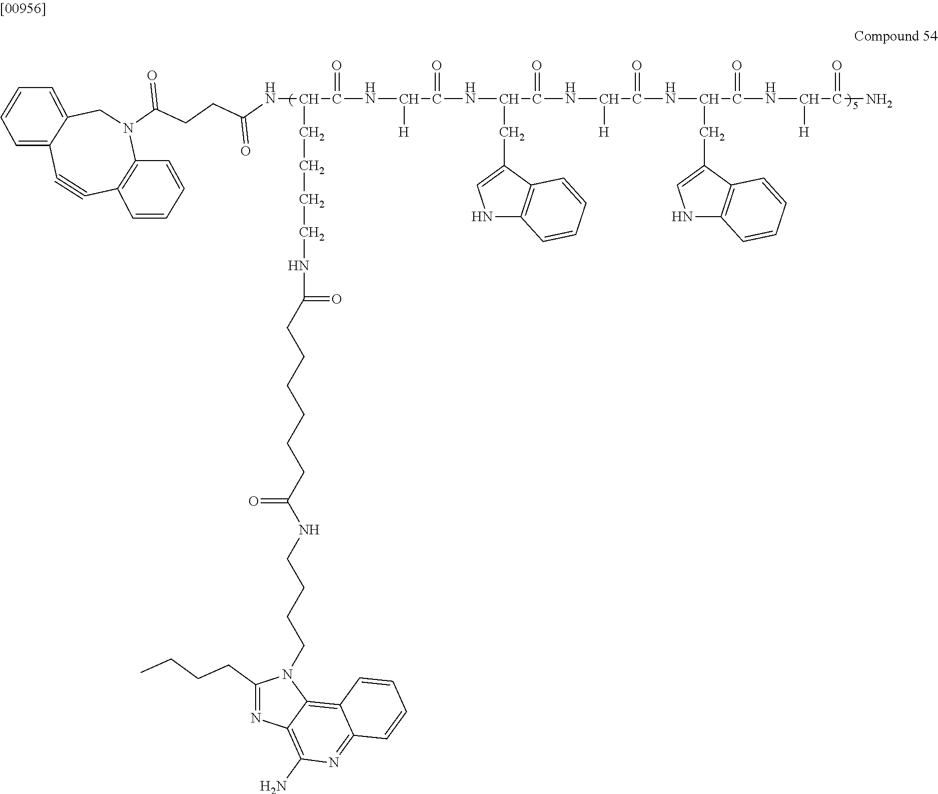

Compound 54 referred to as DBCO-(2BGWGWG)₅, 2BsW₁₀G₁₅ or DBCO-(Glu(2B)₅(Trp)₁₀(Gly)₁₅), was synthesized from an Fmoc-(Lys-Gly-Trp-Gly-Trp-Gly)s-NH₂ (SEQ ID NO:96), peptide precursor that was prepared by solid-phase peptide synthesis and Compound 53. 49.8 mg (0.01 mmol, 1 eq) of Fmoc-(Lys-Gly-Trp-Gly-Trp-Gly)s-NH₂ (SEQ ID NO:96), was dissolved in 0.5 mL of dry DMSO. To this solution was added 0.492 mL of Compound 53 (0.03 mmol, 2.5 eq) as a 40 mg/mL stock solution in dry DMSO. TEA (0.01 mmol, 1 eq) was added and the reaction mixture was stirred at room temperature for 4 hours. Analytical HPLC using a gradient of 45-65% acetonitrile/H2O (0.05% TFA) over 10 minutes showed complete conversion to the penta-substituted intermediate. The reaction was quenched by addition of amino-2-propanol (0.03 mmol, 2.5 eq) and then 0.5 mL of 20% piperidine in DMF was added and the reaction mixture was stirred at room temperature for 30 minutes. The reaction mixture was added to 50 mL of ether and centrifuged at 3000 g at 4° C. for 10 minutes. The product was collected as a solid pellet and then washed twice more with ether, followed by drying under vacuum to yield the deprotected intermediate. 73.4 mg of the deprotected intermediate (0.0131 mmol, 1 eq) was dissolved in 0.5 mL of dry DMSO, followed by the addition of 0.066 mL (0.0196 mmol, 1.5 eq) of DBCO-NHS (40 mg/mL) and TEA (0.0131 mmol, 1 eq). The reaction was stirred for 1 hour at room temperature and then quenched by the addition of amino-2-propanol (0.0196 mmol, 1.5 eq). The product was then precipitated from 50 mL of 1 M HCl and centrifuged at 3000 g at 4° C. for 10 minutes. The product was collected as a solid pellet and then washed once more with 1 M HCl and once more with DI water. The final collected pellet was dried under vacuum to yield 15.1 mg (26% yield) of the final product. MS (ESI) calculated for C₃₁₉H₃₉₆N₇₂O₄₂ m/z 5909.1 found 1183(m/5)⁺.

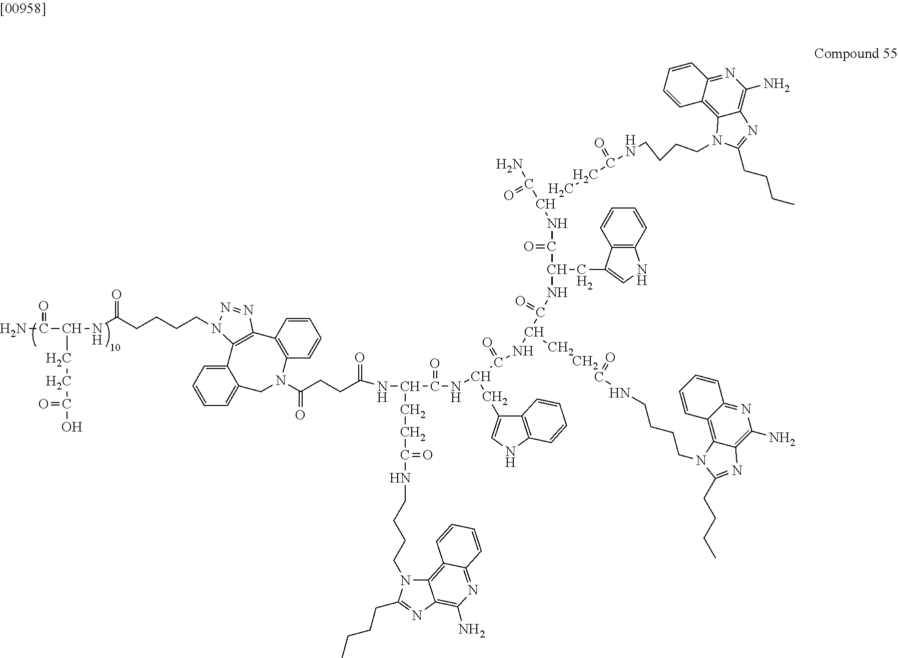

Compound 55, referred to as E₁₀-2B₃W₂, was synthesized using Azido-(Glu)₁₀-NH₂(SEQ ID NO:97) and Compound 42 as the starting materials. 5 mg of Azido-(Glu)₁₀-NH₂ (SEQ ID NO:97), (0.0035 mmol, 1 eq) was dissolved in dry DMSO and 6.77 mg of Compound 42 (0.0035 mmol, 1 eq) as a 40 mg/mL solution in dry DMSO was added. The reaction mixture was stirred overnight at room temperature. Compound 55 was purified on a preparatory HPLC system using a gradient of 25-45% acetonitrile/H₂O (0.05% TFA) over 10 minutes on an Agilent Prep-C18 column, 30×100 mm, 5 μm. The resulting fractions were collected, frozen and then lyophilized to obtain 11.8 mg of a spectroscopically pure (>95% AUC at 254 nm) white powder in quantitative yield. MS (ESI) calculated for m/z 3377.31, found 1127 (M/3)⁺.

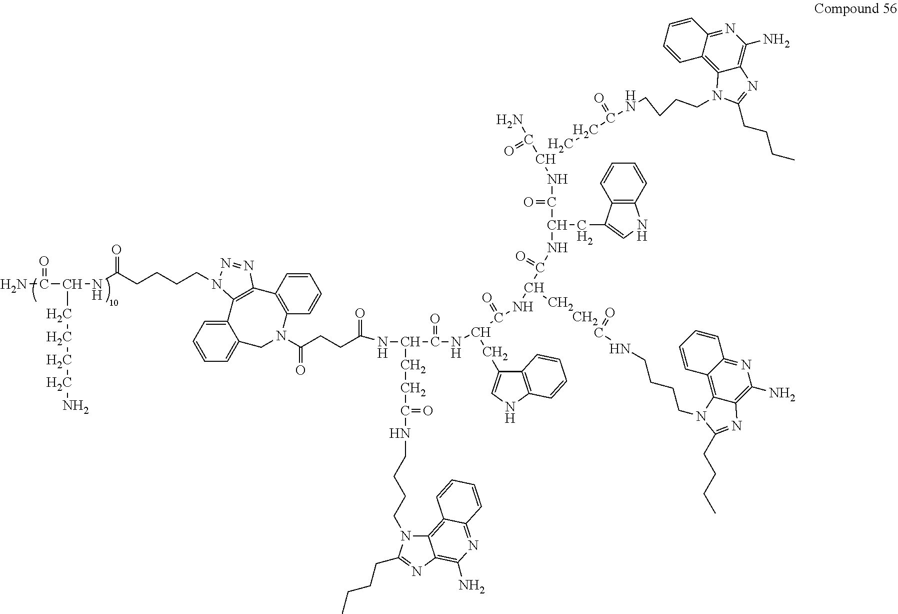

Compound 56, referred to as Kio-2B₃W₂ was synthesized using the same procedure as Compound 55, except Azido-(Lys)₁₀-NH₂ (SEQ ID NO:98), was used as the starting material. Compound 46 was purified on a preparatory HPLC system using a gradient of 20-40% acetonitrile/H₂O (0.05% TFA) over 10 minutes on an Agilent Prep-C18 column, 30×100 mm, 5 m. The resulting fractions were collected, frozen and then lyophilized to obtain a spectroscopically pure (>95% AUC at 254 nm) white powder in quantitative yield. MS (ESI) calculated for m/z 3367.58, found 482 (M/7)⁺.

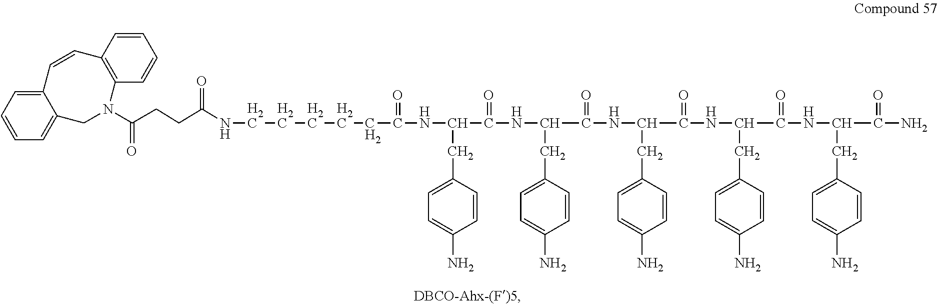 [SMILES: NC(=O)C(Cc1ccc(N)cc1)NC(=O)C(Cc1ccc(N)cc1)NC(=O)C(Cc1ccc(N)cc1)NC(=O)C(Cc1ccc(N)cc1)NC(=O)C(Cc1ccc(N)cc1)NC(=O)CCCCCNC(=O)CCC(=O)N1Cc2ccccc2/C=C\c2ccccc21]

Compound 57, referred to as DBCO-Ahx-F′ ₅ or Ahx-F′₅ was synthesized by reacting 400 mg (0.4 mmol, 1 eq) of the precursor Ahx-(F′)₅—NH₂, which was prepared by solid phase peptide synthesis, with 171.05 mg of DBCO-NHS (0.4 mmol, 1.0 eq) and 258.1 mg of Triethylamine (2.55 mmol, 6.0 eq) in 3.7 mL of DMSO. The DBCO-NHS was added in 4 increments of 0.25 eq. The reaction was run overnight at room temperature and HPLC indicated that the reaction was complete by 24 hours. Compound 57 was purified on a preparatory HPLC system using a gradient of 13-43% acetonitrile/H₂O (0.05% TFA) over 12 minutes on an Agilent Prep-C18 column, 50×100 mm, 5 μm. The product eluted at ˜5.7 minutes and the resulting fractions were collected, frozen and then lyophilized to obtain 217.0 mg (41.5% yield) of a spectroscopically pure (>95% AUC at 254 nm) white/yellow powder. MS (ESI) calculated for C₇₀H₇₆N₁₂O₉ m/z 1228.59, found 1228.7 (M+H)⁺.

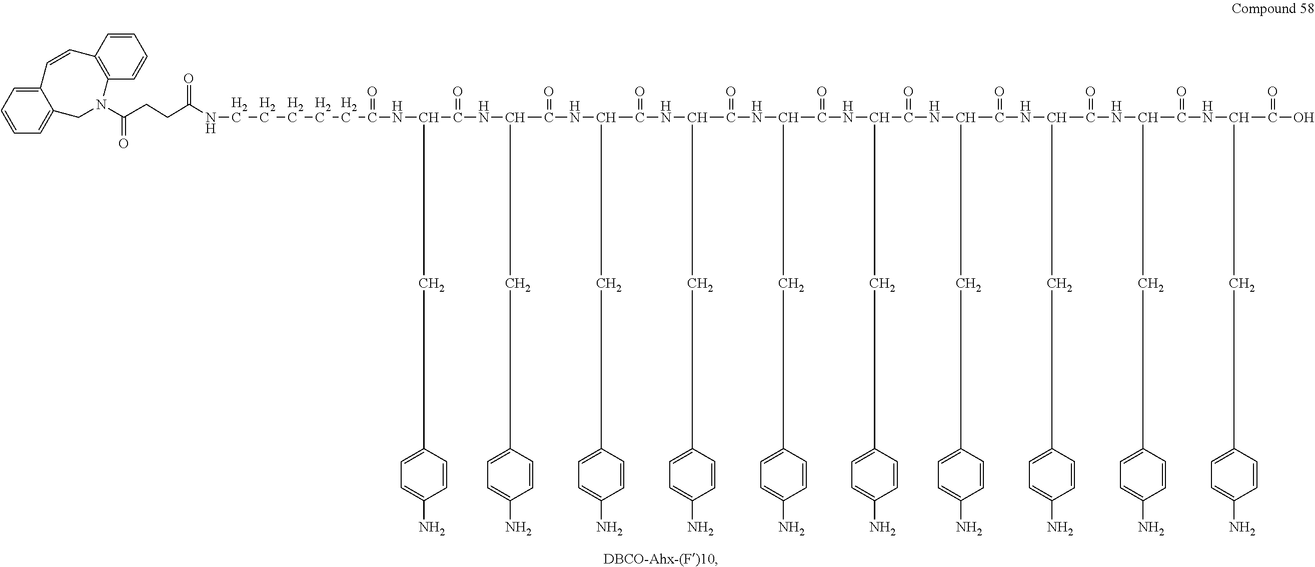 [SMILES: Nc1ccc(CC(NC(=O)C(Cc2ccc(N)cc2)NC(=O)C(Cc2ccc(N)cc2)NC(=O)C(Cc2ccc(N)cc2)NC(=O)C(Cc2ccc(N)cc2)NC(=O)C(Cc2ccc(N)cc2)NC(=O)C(Cc2ccc(N)cc2)NC(=O)C(Cc2ccc(N)cc2)NC(=O)C(Cc2ccc(N)cc2)NC(=O)C(Cc2ccc(N)cc2)NC(=O)CCCCCNC(=O)CCC(=O)N2Cc3ccccc3/C=C\c3ccccc32)C(=O)O)cc1]

Compound 58, referred to as DBCO-Ahx-F′₁₀ or Ahx-F′₁₀ was synthesized by reacting 450 mg (0.26 mmol, 1 eq) of the precursor Ahx-(F′)₁₀—NH₂ (SEQ ID NO:99), which was prepared by solid phase peptide synthesis, with 103.4 mg of DBCO-NHS (0.26 mmol, 1.0 eq) and 286.1 mg of Triethylamine (2.83 mmol, 11.0 eq) in 3.3 mL of DMSO. The DBCO-NHS was added in 4 increments of 0.25 eq. The reaction was run overnight at room temperature and HPLC indicated that the reaction was complete by 24 hours. Compound 58 was purified on a preparatory HPLC system using a gradient of 15-45% acetonitrile/H₂O (0.05% TFA) over 12 minutes on an Agilent Prep-C18 column, 50×100 mm, 5 μm. The product eluted at ˜5.1 minutes and the resulting fractions were collected, frozen and then lyophilized to obtain 265.4 mg (50.6% yield) of a spectroscopically pure (>95% AUC at 254 nm) red/copper powder. MS (ESI) calculated for C₂₀₅H₂₂₆N₄₂O₂₄ m/z 3659.78, found 1221.3 (M+3H)⁺.

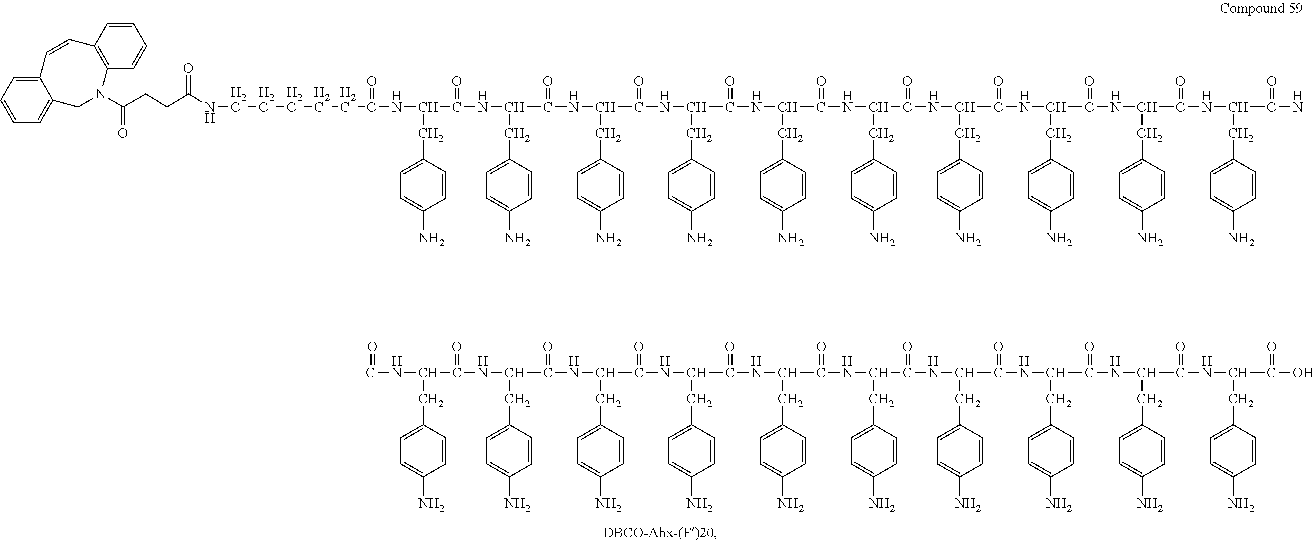

Compound 59, referred to as DBCO-Ahx-F′₂₀ or Ahx-F′₂₀ DBCO-Ahx-(F′)₂₀ (SEQ ID NO:100) was synthesized by reacting 480 mg (0.14 mmol, 1 eq) of the precursor Ahx-(F′)₂₀—NH₂ (SEQ ID NO:101), which was prepared by solid phase peptide synthesis, with 57.3 mg of DBCO-NHS (0.14 mmol, 1.0 eq) and 302.4 mg of Triethylamine (2.99 mmol, 21.0 eq) in 3.0 mL of DMSO. The DBCO-NHS was added in 4 increments of 0.25 eq. The reaction was run overnight at room temperature and HPLC indicated that the reaction was complete by 24 hours. Compound 59 was purified on a preparatory HPLC system using a gradient of 13-43% acetonitrile/H₂O (0.05% TFA) over 12 minutes on an Agilent Prep-C18 column, 50×100 mm, 5 μm. The product eluted at ˜5.5 minutes and the resulting fractions were collected, frozen and then lyophilized to obtain 106.6 mg (20.5% yield) of a spectroscopically pure (94.4% AUC at 254 nm) brown/copper powder. MS (ESI) calculated for C₁₁₅H₁₂₆N₂₂O₁₄ m/z 2039.99, found 1020.5 (M+2H)⁺.

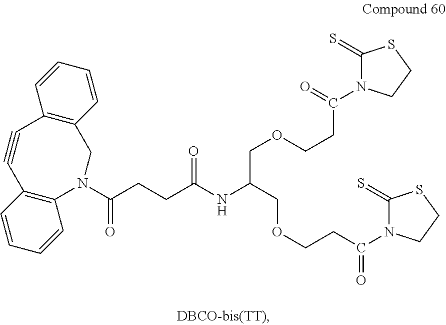 [SMILES: O=C(CCC(=O)N1Cc2ccccc2C#Cc2ccccc21)NC(COCCC(=O)N1CCSC1=S)COCCC(=O)N1CCSC1=S]

Compound 60, referred to as DBCO-2-Amino-1,3-bis(carboxylethoxy)propane(TT)₂ or DBCO-bis(TT) was synthesized by reacting 385.6 mg (0.74 mmol, 1 eq) of the precursor DBCO-2-Amino-1,3-bis(carboxylethoxy)propane, with 193.4 mg of 2-Thiazoline-2-thiol (1.62 mmol, 2.2 eq) and 367.5 mg of 1-Ethyl-3-(3-dimethylaminopropyl) carbodiimide (1.92 mmol, 2.6 eq) in and 4-Dimethylaminopyridine in 4.0 mL of DCM. The reaction was run overnight at room temperature and HPLC indicated that the reaction was complete by 24 hours. The product eluted at 6.8 minutes on an Agilent analytical C18 column, 4.6×100 mm, 2.7 μm. Compound 60 was extracted with ethyl acetate and 1M HCl and was dried on the rotovap to obtain 317.1 mg (59.3% yield) of an impure (27.0% AUC at 254 nm) yellow powder. MS (ESI) calculated for C₃₄H₃₆N₄O₆S₄ m/z 724.15, found 725.3 (M+H)+

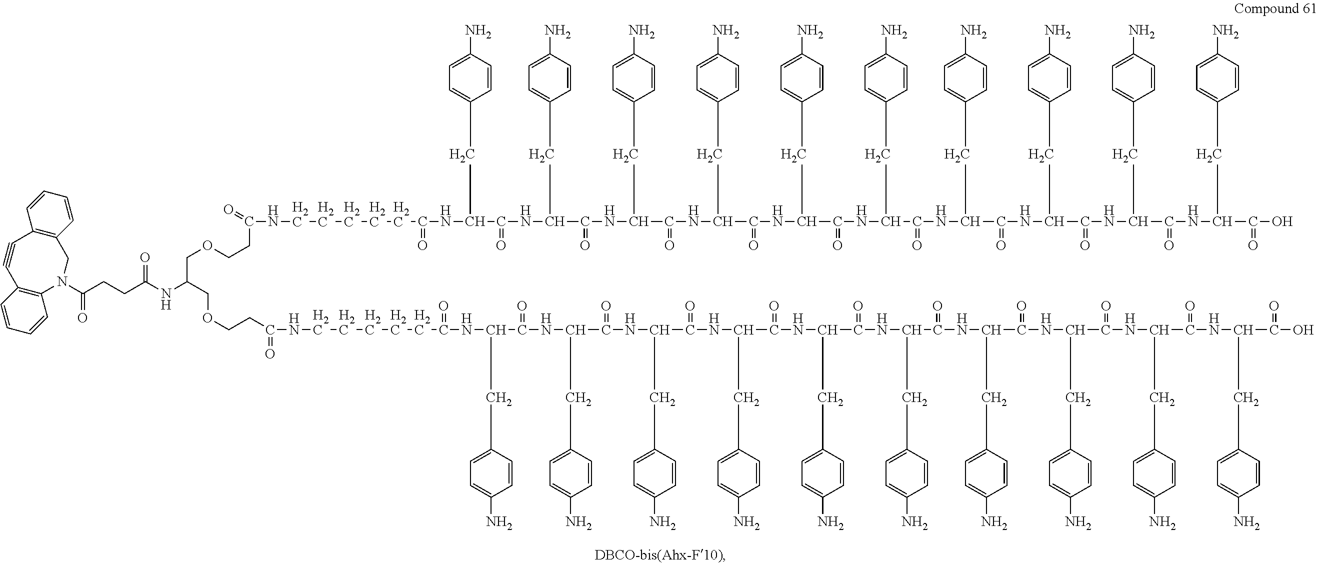

Compound 61, referred to as DBCO-2-Amino-1,3-bis(carboxylethoxy)propane(Ahx-F′ 10)₂ or DBCO-bis(Ahx-F′ 10) was synthesized by reacting 13.0 mg (0.018 mmol, 1 eq) of the precursor DBCO-2-Amino-1,3-bis(carboxylethoxy)propane(TT)₂, Compound 61, with 314.2 mg of Ahx-(F′)₁₀—NH₂ (SEQ ID NO: 102) (0.18 mmol, 10 eq) that was prepared by solid phase peptide synthesis and 199.5 mg of Triethylamine (1.97 mmol, 11.0 eq) in 1.8 mL of DMSO. The reaction was run overnight at room temperature and HPLC indicated that the reaction was complete by 24 hours. Compound 61 was purified on a preparatory HPLC system using a gradient of 5-25-35% acetonitrile/H₂O (0.05% TFA) over 14 minutes on an Agilent Prep-C18 column, 50×100 mm, 5 m. The product eluted at ˜9.8 minutes and the resulting fractions were collected, frozen and then lyophilized to obtain 19.16 mg (26.8% yield) of a spectroscopically pure (83.4% AUC at 254 nm) orange powder. MS (ESI) calculated for C₂₂₀H₂₅₂N₄₄O₃₀ m/z 3989.95, found 1330.8 (M+3H)⁺.

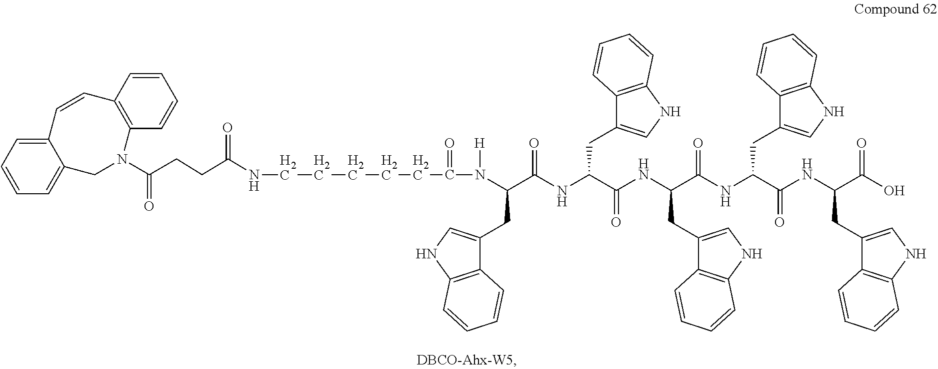 [SMILES: O=C(CCC(=O)N1Cc2ccccc2/C=C\c2ccccc21)NCCCCCC(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)O]

Compound 62, referred to as DBCO-Ahx-W₅ was synthesized by reacting 14.2 mg (0.035 mmol, 1 eq) of the precursor DBCO-NHS, with 37.5 mg of Ahx-(W)₅—NH₂ (SEQ ID NO: 103) (0.035 mmol, 1 eq) that was prepared by solid phase peptide synthesis and 3.93 mg of Triethylamine (0.039 mmol, 1.1 eq) in 0.5 mL of DMSO. The reaction was run overnight at room temperature and HPLC indicated that the reaction was complete by 24 hours. Compound 62 was crashed out in twice 1M HCL and once in H₂O to obtain 34.3 (71.9% yield) of a spectroscopically pure (92.6% AUC at 254 nm) pink powder. MS (ESI) calculated for C₈₀H₇₆N₁₂O₉ m/z 1348.59, found 1348.4 (M+H)⁺.

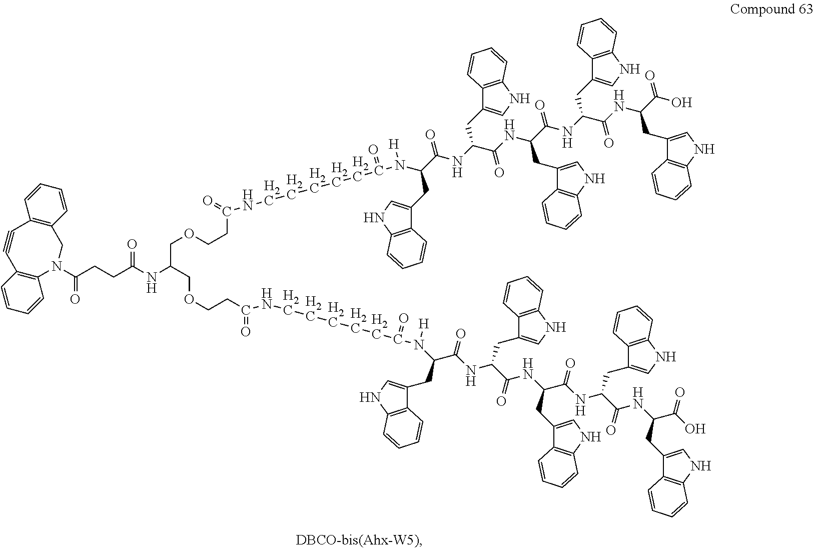 [SMILES: O=C(CCOCC(COCCC(=O)NCCCCCC(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)O)NC(=O)CCC(=O)N1Cc2ccccc2C#Cc2ccccc21)NCCCCCC(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)O]

Compound 63, referred to as DBCO-2-Amino-1,3-bis(carboxylethoxy)propane(Ahx-W₅)₂ or DBCO-bis-(Ahx-W₅) was synthesized by reacting 13.0 mg (0.018 mmol, 1 eq) of the precursor DBCO-2-Amino-1,3-bis(carboxylethoxy)propane(TT)₂, with 41.3 mg of Ahx-(W)₅—NH₂ (0.039 mmol, 2.2 eq) that was prepared by solid phase peptide synthesis and 9.1 mg of Triethylamine (0.09 mmol, 2.3 eq) in 0.3 mL of DMSO. The reaction was run overnight at room temperature and HPLC indicated that the reaction was complete by 24 hours. Compound 63 was purified on a preparatory HPLC system using a gradient of 15-60-90% acetonitrile/H₂O (0.05% TFA) over 16 minutes on an Agilent Prep-C18 column, 30×100 mm, 5 μm. The product eluted at ˜12.7 minutes and the resulting fractions were collected, frozen and then lyophilized to obtain 12.5 mg (30.8% yield) of a spectroscopically pure (>95% AUC at 254 nm) pink powder. MS (ESI) calculated for C₁₅₀H₁₅₂N₂₄O₂₀ m/z 2609.16, found 1305.0 (M+2H)⁺.

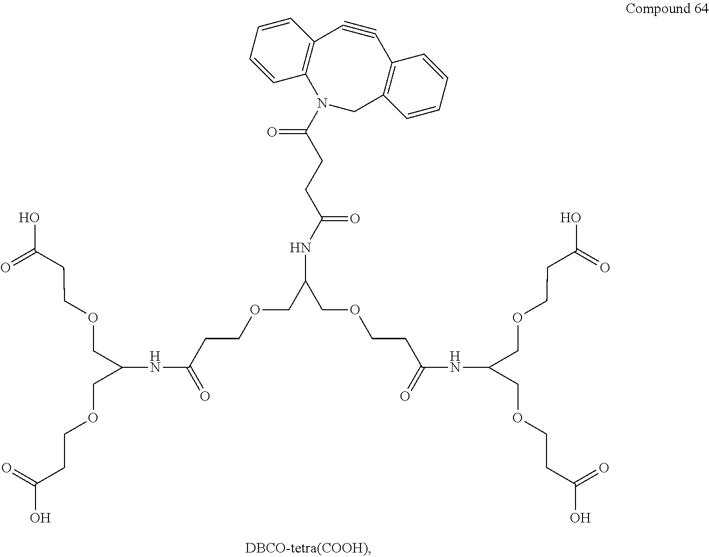 [SMILES: O=C(O)CCOCC(COCCC(=O)O)NC(=O)CCOCC(COCCC(=O)NC(COCCC(=O)O)COCCC(=O)O)NC(=O)CCC(=O)N1Cc2ccccc2C#Cc2ccccc21]

Compound 64, referred to as DBCO-2-Amino-1,3-bis(carboxylethoxy)propane(COOH)₄ or DBCO-tetra(COOH), was synthesized by reacting 250 mg (0.34 mmol, 1.1 eq) of the precursor Compound 60 with 170 mg of DBCO-2-Amino-1,3-bis(carboxylethoxy)propane (0.6 mmol, 2 eq) and 190 mg of TEA (1.9 mmol, 6 eq) in 2.5 mL of DMF. The reaction was run for 1 hour at room temperature and HPLC indicated the reaction was complete. MS (ESI) calculated for C₄₆H₆₀N₄O₁₈ m/z 956.4, found 957.2 (M+H)⁺.

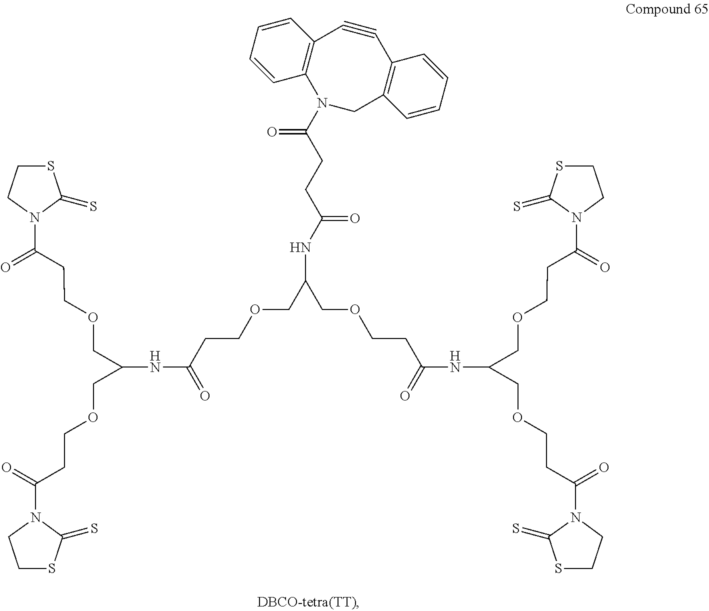 [SMILES: O=C(CCOCC(COCCC(=O)NC(COCCC(=O)N1CCSC1=S)COCCC(=O)N1CCSC1=S)NC(=O)CCC(=O)N1Cc2ccccc2C#Cc2ccccc21)NC(COCCC(=O)N1CCSC1=S)COCCC(=O)N1CCSC1=S]

Compound 65, referred to as DBCO-2-Amino-1,3-bis(carboxylethoxy)propane(TT)₄ or DBCO-tetra(TT), was synthesized by reacting 178 mg (0.19 mmol, 1 eq) of the precursor Compound 64 with 115 mg of 2-Thiazoline-2-thiol (0.96 mmol, 5.2 eq). TEA (2.98 mmol, 16 eq) was added and the reaction mixture was cooled in an ice bath for 5 minutes. 310 mg of HATU (0.8 mmol, 4.4 eq) was added and the reaction mixture was stirred in an ice bath. The progress of the reaction was monitored by LC-MS. After 2 hours, the reaction was complete. Compound 65 was crashed out once in 1M HCl and once in H₂O. The resulting solid was dissolved in ACN and dried on rotovap to obtain 215 mg (85.0% yield) of an impure (53.0% AUC at 254 nm) yellow/brown oil. MS (ESI) calculated for C₅₈H₇₂N₈O₁₄S₈ m/z 1360.3, found 1361.0 (M+H)⁺.

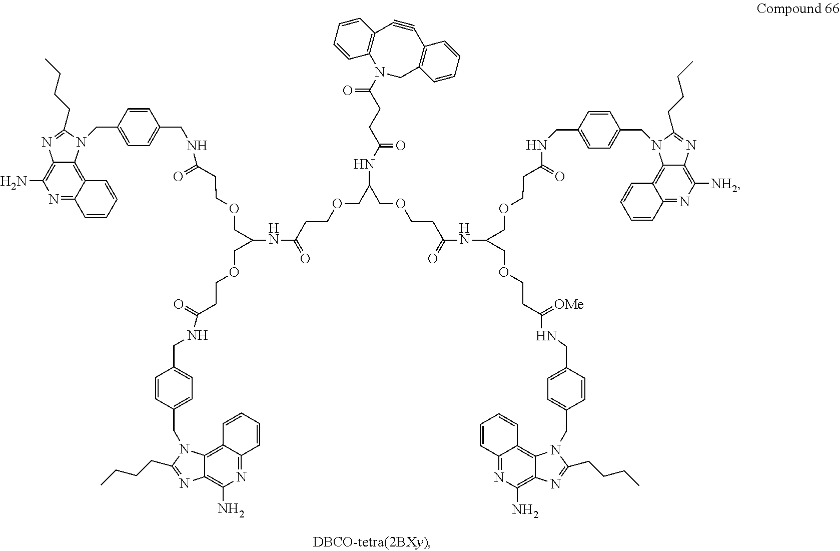

Compound 66, referred to as DBCO-2-Amino-1,3-bis(carboxylethoxy)propane(2BXy)₄ or DBCO-tetra(2BXy), was synthesized by reacting 16 mg (0.012 mmol, 1 eq) of the precursor Compound 65 with 17 mg of Compound 36 (0.047 mmol, 4 eq) and TEA (0.047 mmol, 4 eq) in 0.5 mL DMSO. The progress of the reaction was monitored by HPLC. After 1 hour, the reaction was complete. Compound 66 was purified on a preparatory HPLC system using a gradient of 38-48% acetonitrile/H₂O (0.05% TFA) over 12 minutes on an Agilent Prep-C18 column, 30×100 mm, 5 μm. The product eluted at ˜4.0 minutes and the resulting fractions were collected, frozen and then lyophilized to obtain 13.6 mg (49.8% yield) of a spectroscopically pure (98.3% AUC at 254 nm) white powder. MS (ESI) calculated for C₁₃₄H₁₅₂N₂₄O₁₄ m/z 2321.2, found 775.0 (M/3+H)⁺.

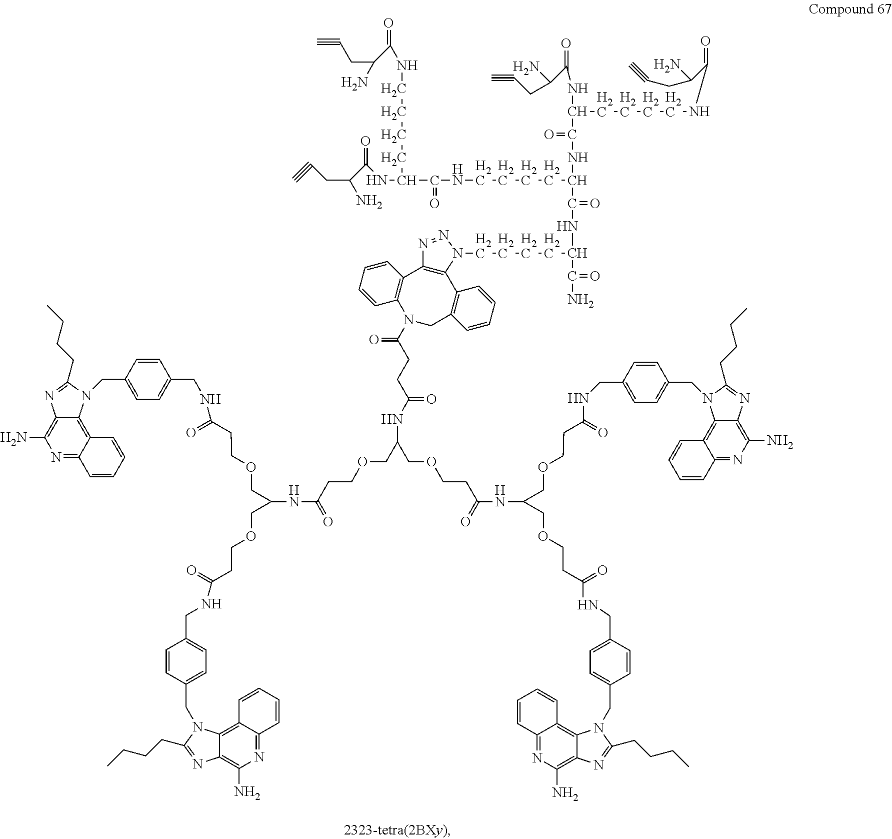 [SMILES: C#CCC(N)C(=O)NCCCCC(NC(=O)C(N)CC#C)C(=O)NCCCCC(NC(=O)C(CCCCNC(=O)C(N)CC#C)NC(=O)C(N)CC#C)C(=O)NC(CCCCn1nnc2c1-c1ccccc1CN(C(=O)CCC(=O)NC(COCCC(=O)NC(COCCC(=O)NCc1ccc(Cn3c(CCCC)nc4c(N)nc5ccccc5c43)cc1)COCCC(=O)NCc1ccc(Cn3c(CCCC)nc4c(N)nc5ccccc5c43)cc1)COCCC(=O)NC(COCCC(=O)NCc1ccc(Cn3c(CCCC)nc4c(N)nc5ccccc5c43)cc1)COCCC(=O)NCc1ccc(Cn3c(CCCC)nc4c(N)nc5ccccc5c43)cc1)c1ccccc1-2)C(N)=O]

Compound 67, referred to as {propargyl}₄K2K{Lys(N₃)}-DBCO-2-Amino-1,3-bis(carboxylethoxy)propane(2BXy)₄ or 2323-tetra(2BXy), was synthesized by reacting 16 mg of {propargyl}₄K₂K{Lys(N₃)}, which was prepared by solid phase peptide synthesis, (0.17 mmol, 1 eq) and 39 mg of Compound 66 (0.017 mmol, 1 eq) in 1.0 mL dry DMSO. The reaction mixture was stirred overnight at room temperature. HPLC indicated that the reaction was complete. Compound 67 was purified on a preparatory HPLC system using a gradient of 20-50% acetonitrile/H₂O (0.05% TFA) over 12 minutes on an Agilent Prep-C18 column, 30×100 mm, 5 m. The product eluted at ˜6.5 minutes and the resulting fractions were collected, frozen and then lyophilized to obtain 26 mg (47.5% yield) of a spectroscopically pure (98.6% AUC at 254 nm) white powder. MS (ESI) calculated for C₁₇₈H₂₂₁N₃₉O₂₂ m/z 3256.7, found 1087.0 (M/3+H)⁺.

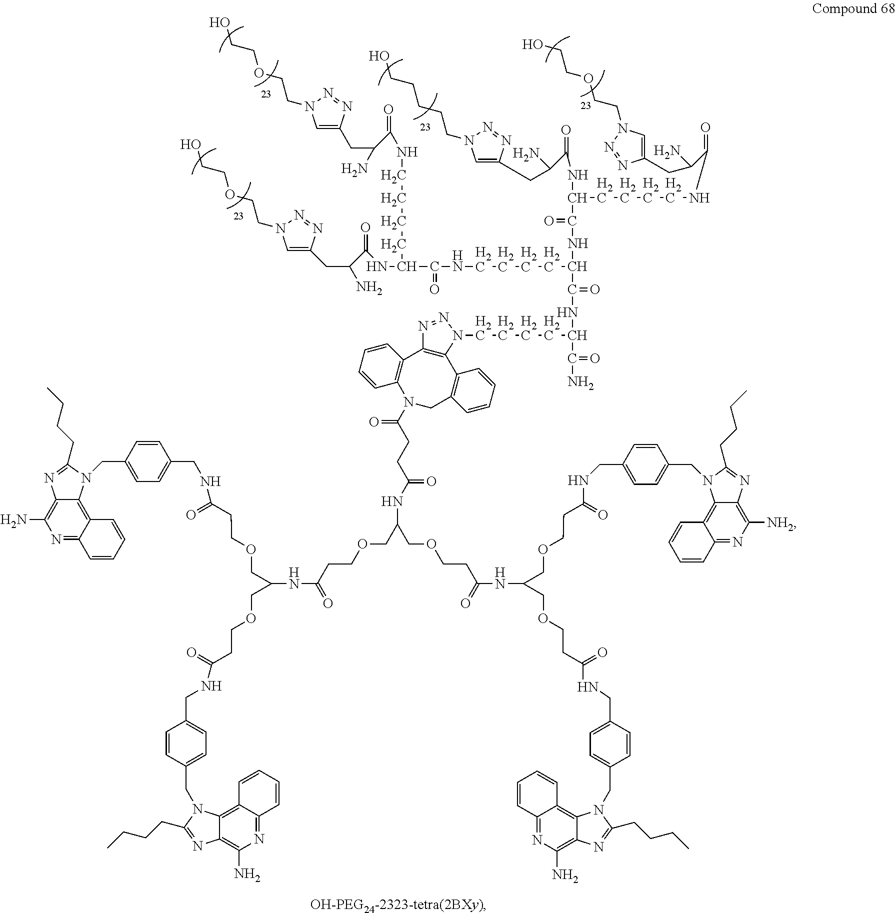

Compound 68, referred to as {OH-PEG₂₄}₄-{propargyl}₄K₂K{Lys(N₃)}-DBCO-2- Amino 1,3-bis(carboxylethoxy)propane(2BXy)₄ or OH-PEG₂₄-2323-tetra(2BXy), was synthesized by reacting 3.5 mg Compound 67 (0.0011 mmol, 1 eq) with 4.7 mg of alpha-Azido-omega-hydroxy 24(ethylene glycol) (0.0043 mmol, 4 eq) in 0.058 mL H₂O and 0.117 mL DMSO. To the reaction mixture, 0.8 mg Sodium Ascorbate (0.0043 mmol and 4 eq) was added. 1.1 mg Copper Sulfate Pentahydrate (0.0043 mmol, 4 eq) and 1.9 mg tris-hydroxypropyltriazolylmethylamine (0.0043 mmol, 4 eq) were combined in a separate vial, and then added to the reaction mixture. The reaction mixture was stirred overnight at room temperature. LC-MS indicated that the reaction was complete. Compound 68 was purified by dialysis with a Regenerated Cellulose membrane, MWCO: 2 kDa, with solvent changes of 1:1 H₂O/MeOH with 0.01% EDTA (2×), 1:1 H₂O/MeOH (1×), and MeOH (2×). Sample collected and dried on rotovap to obtain 5.8 mg (70.5% yield) of a spectroscopically pure (99.3% AUC at 254 nm) blue solid. MS (ESI) calculated for C₃₇₀H₆₀₉N₅₁O₁₁₈ m/z 7655.3, found 1277.8 (M/6)⁺.

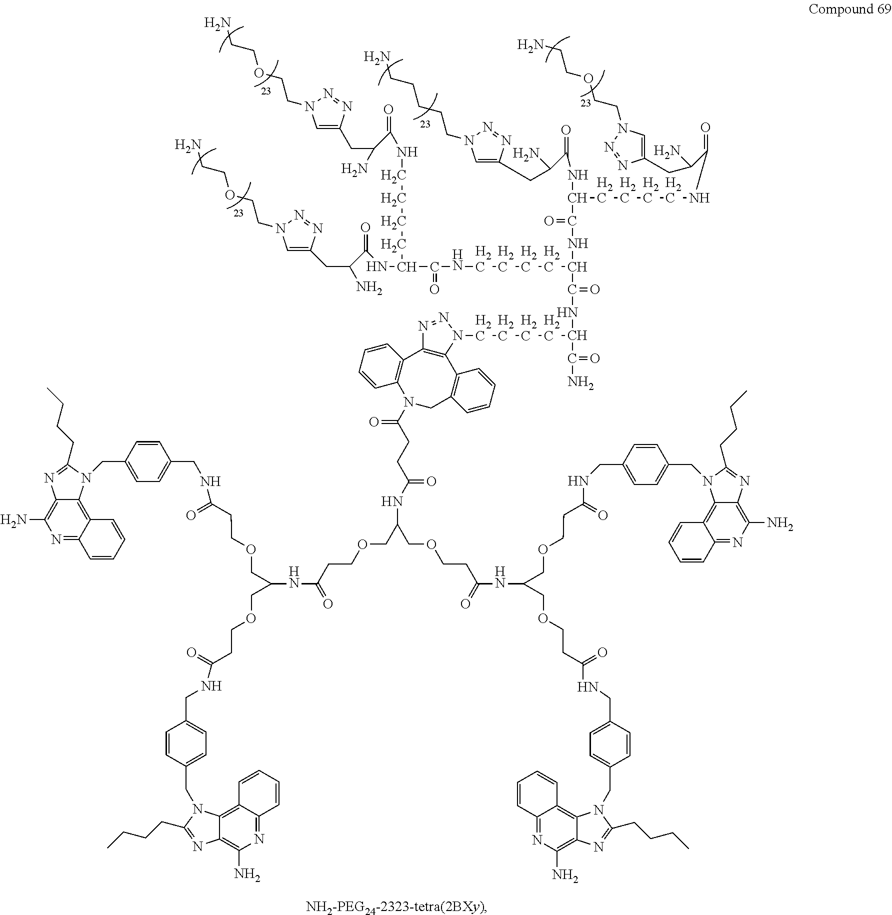

Compound 69, referred to as {NH₂—PEG₂₄}4-{propargyl}₄K₂K{Lys(N₃)}-DBCO-2-Amino-1,3-bis(carboxylethoxy)propane(2BXy)₄ or NH₂—PEG2₄-2323-tetra(2BXy), was synthesized and purified using the same procedure as Compound 68, except alpha-Azido-omega-amino 23(ethylene glycol) was used as the starting material. Upon collecting purified sample and drying on rotovap, 4.8 mg (58.4% yield) of a spectroscopically pure (95.5% AUC at 254 nm) green solid was obtained. MS (ESI) calculated for C₃₇₀H₆₁₃N₅₅O₁₁₄ m/z 7651.4, found 1276.8 (M/6+H)⁺.

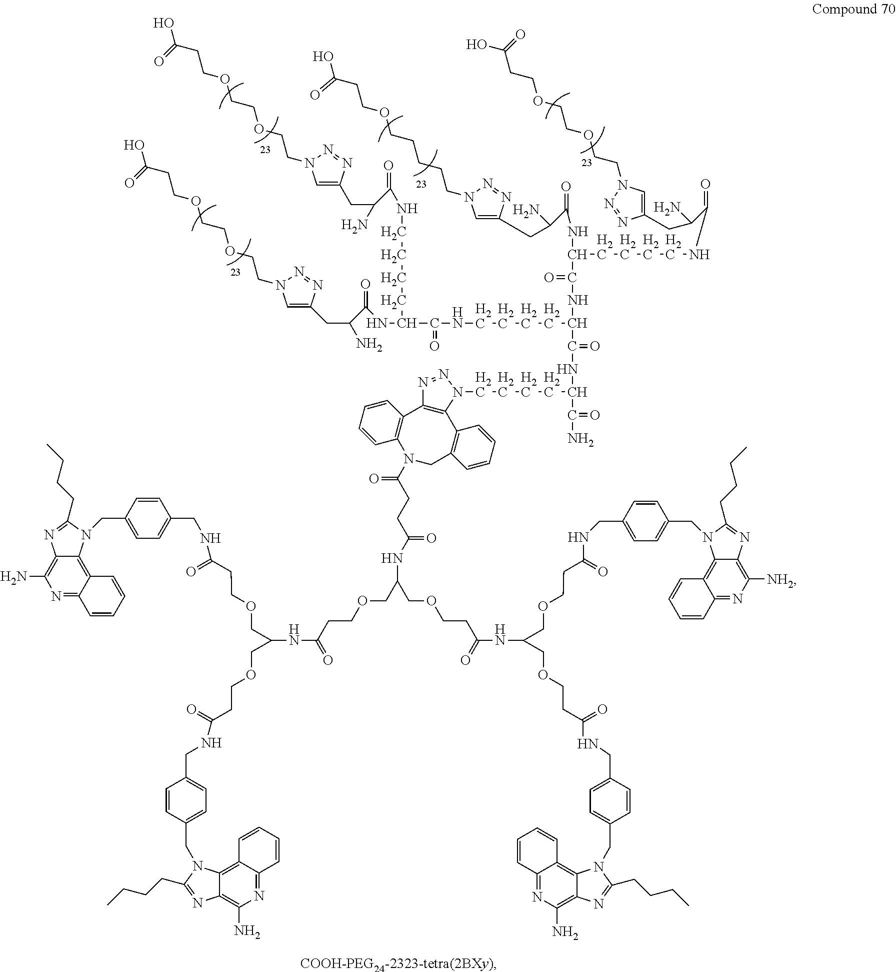

Compound 70, referred to as {COOH-PEG₂₄}4-{propargyl}₄K₂K{Lys(N₃)}-DBCO-2-Amino-1,3-bis(carboxylethoxy)propane(2BXy)₄ or COOH-PEG₂₄-2323-tetra(2BXy), was synthesized and purified using the same procedure as Compound 68, except alpha-Azido-omega-(propionic acid) 24(ethylene glycol) was used as the starting material. Upon collecting purified sample and drying on rotovap, 6.0 mg (70.3% yield) of a spectroscopically pure (98.0% AUC at 254 nm) blue solid was obtained. MS (ESI) calculated for C₃₈₂H₆₂₅N₅₁O₁₂₆ m/z 7943.1, found 1325.4 (M/6+H)⁺.

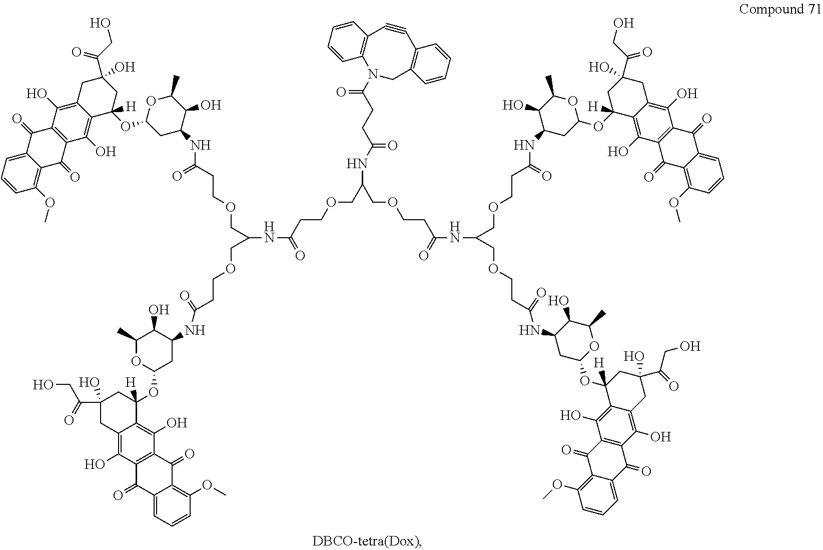 [SMILES: COc1cccc2c1C(=O)c1c(O)c3c(c(O)c1C2=O)C[C@@](O)(C(=O)CO)C[C@@H]3O[C@H]1C[C@H](NC(=O)CCOCC(COCCC(=O)N[C@H]2C[C@H](O[C@H]3C[C@](O)(C(=O)CO)Cc4c(O)c5c(c(O)c43)C(=O)c3c(OC)cccc3C5=O)O[C@@H](C)[C@H]2O)NC(=O)CCOCC(COCCC(=O)NC(COCCC(=O)N[C@@H]2CC(O[C@@H]3C[C@@](O)(C(=O)CO)Cc4c(O)c5c(c(O)c43)C(=O)c3c(OC)cccc3C5=O)O[C@H](C)[C@@H]2O)COCCC(=O)N[C@@H]2C[C@@H](O[C@@H]3C[C@@](O)(C(=O)CO)Cc4c(O)c5c(c(O)c43)C(=O)c3c(OC)cccc3C5=O)O[C@H](C)[C@@H]2O)NC(=O)CCC(=O)N2Cc3ccccc3C#Cc3ccccc32)[C@H](O)[C@H](C)O1]

Compound 71, referred to as DBCO-2-Amino-1,3-bis(carboxylethoxy)propane(Doxorubicin)₄ or DBCO-tetra(Dox), was synthesized by reacting 23 mg (0.017 mmol, 1 eq) of the precursor Compound 65 with 40 mg of Doxorubicin Hydrochloride (0.069 mmol, 4 eq) and TEA (0.138 mmol, 8 eq) in 1.5 mL DMSO. The progress of the reaction was monitored by HPLC. After 1 hour, the reaction was complete. Compound 71 was purified on a preparatory HPLC system using a gradient of 38-48% acetonitrile/H₂O (0.05% TFA) over 12 minutes on an Agilent Prep-C18 column, 30×100 mm, 5 μm. The product eluted at ˜7.5 minutes and the resulting fractions were collected, frozen and then lyophilized to obtain 5.3 mg (20.1% yield) of a spectroscopically pure (95.9% AUC at 254 nm) red powder. MS (ESI) calculated for C₁₅₄H₁₆₈N₈O₅₈ m/z 3059.0.

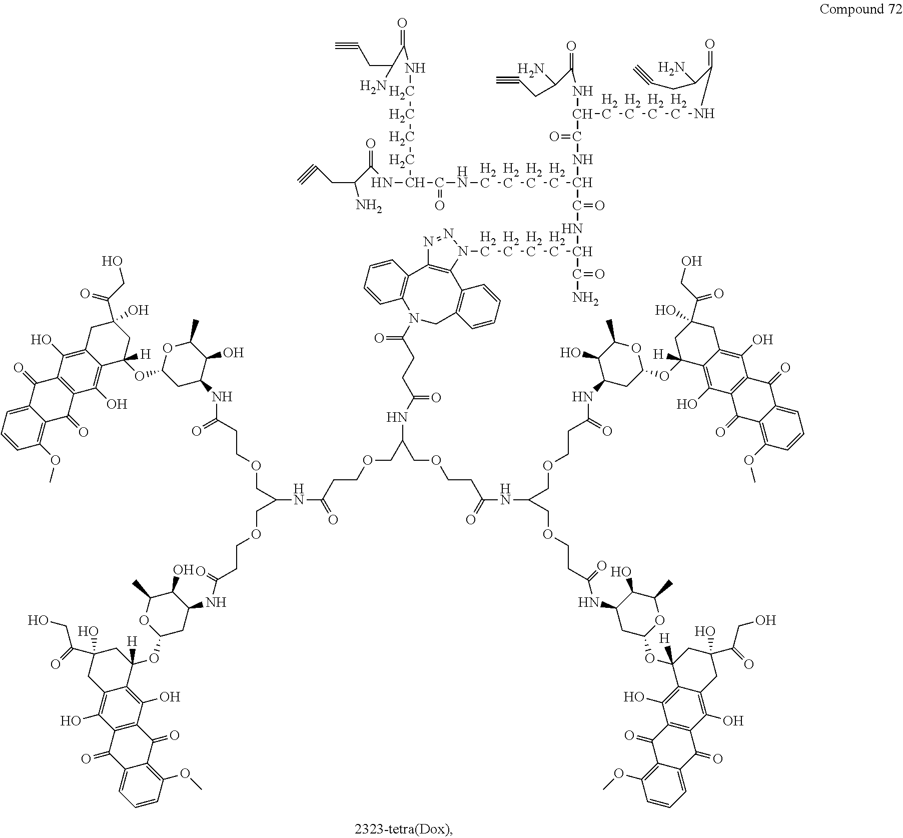

Compound 72, referred to as {propargyl}₄K₂K{Lys(N₃)}-DBCO-2-Amino-1,3-bis(carboxylethoxy)propane(Doxorubicin)₄ or 2323-tetra(Dox), was synthesized by reacting 16 mg of {propargyl}₄K₂K{Lys(N₃)}(0.17 mmol, 1 eq) and 53 mg of Compound 71 (0.017 mmol, 1 eq) in 2.0 mL dry DMSO. The reaction mixture was stirred overnight at room temperature. HPLC indicated that the reaction was complete. Compound 72 was purified on a preparatory HPLC system using a gradient of 25-45% acetonitrile/H₂O (0.05% TFA) over 12 minutes on an Agilent Prep-C18 column, 30×100 mm, 5 μm. The product eluted at ˜4.6 minutes and the resulting fractions were collected, frozen and then lyophilized to obtain 28 mg (40.9% yield) of a spectroscopically pure (91.3% AUC at 254 nm) red/orange powder. MS (ESI) calculated for C₁₉₈H₂₃₇N₂₃O₆₆ m/z 3995.2, found 1332.8 (M/3+H)⁺.

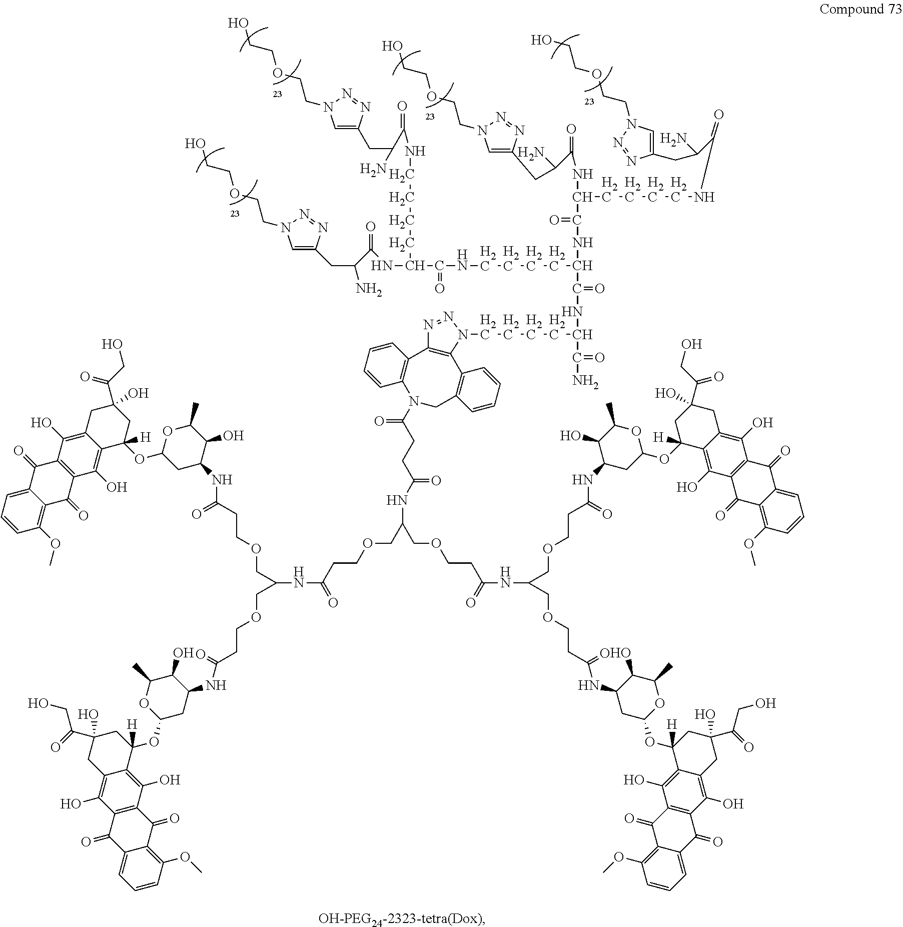

Compound 73, referred to as {OH-PEG₂₄}₄-{propargyl}₄K₂K{Lys(N₃)}-DBCO-2-Amino-1,3-bis(carboxylethoxy)propane(Doxorubucin)₄ or OH-PEG₂₄-2323-tetra(Dox), was synthesized by reacting 3.5 mg Compound 72 (0.0011 mmol, 1 eq) with 4.7 mg of alpha-Azido-omega-hydroxy 24(ethylene glycol) (0.0043 mmol, 4 eq) in 0.054 mL H₂O and 0.109 mL DMSO. To the reaction mixture, 0.7 mg Sodium Ascorbate (0.0035 mmol and 4 eq) was added. 0.9 mg Copper Sulfate Pentahydrate (0.0035 mmol, 4 eq) and 1.5 mg tris-hydroxypropyltriazolylmethylamine (0.0035 mmol, 4 eq) were combined in a separate vial, and then added to the reaction mixture. The reaction mixture was stirred overnight at room temperature. LC-MS indicated that the reaction was complete. Compound 73 was purified by dialysis with a Regenerated Cellulose membrane, MWCO: 2 kDa, with solvent changes of 1:1 H₂O/MeOH with 0.01% EDTA (2×), 1:1 H₂O/MeOH (1×), and MeOH (2×). Sample collected and dried on rotovap to obtain 5.1 mg (69.3% yield) of a spectroscopically pure (95.8% AUC at 254 nm) purple solid. MS (ESI) calculated for C₃₉₀H₆₂₅N₃₅O₁₆₂ m/z 8396.4, found 1399.8 (M/6+H)⁺.

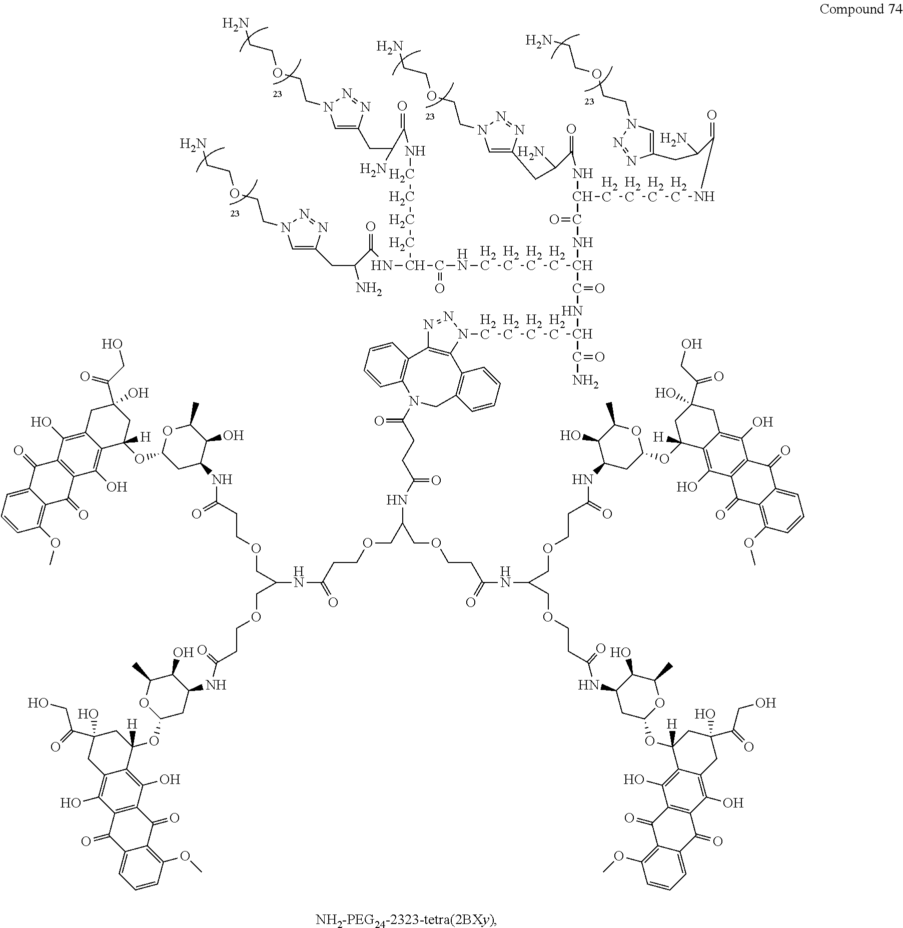

Compound 74, referred to as {NH₂—PEG₂₄}4-{propargyl}₄K₂K{Lys(N₃)}-DBCO-2-Amino-1,3-bis(carboxylethoxy)propane(2BXy)₄ or NH₂—PEG₂₄-2323-tetra(2BXy), was synthesized and purified using the same procedure as Compound 73, except alpha-Azido-omega-amino 23(ethylene glycol) was used as the starting material. Upon collecting purified sample and drying on rotovap, 5.0 mg (68.0% yield) of a spectroscopically impure (67.7% AUC at 254 nm) purple solid was obtained. MS (ESI) calculated for C₃₉₀H₆₂₉N₃₉O₁₅₈ m/z 8387.2, found 1399.2 (M/6+H)⁺.

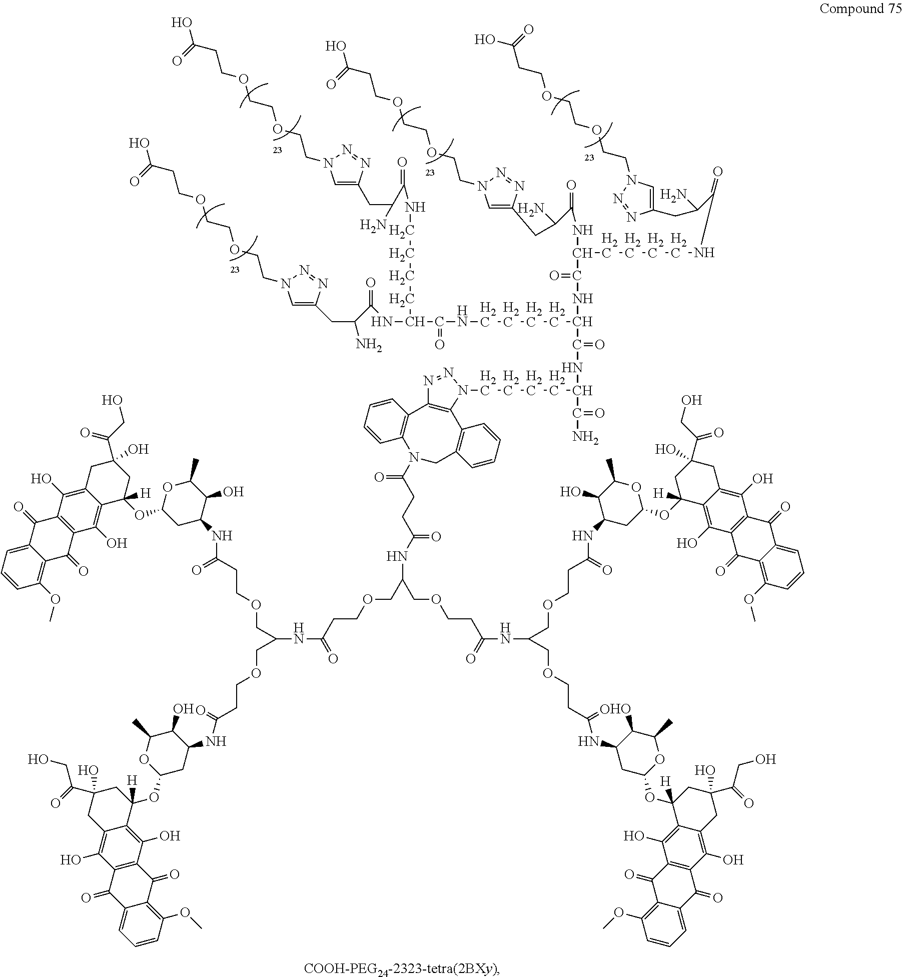

Compound 75, referred to as {COOH-PEG₂₄}4-{propargyl}₄K₂K{Lys(N₃)}-DBCO-2-Amino-1,3-bis(carboxylethoxy)propane(2BXy)₄ or COOH-PEG₂₄-2323-tetra(2BXy), was synthesized and purified using the same procedure as Compound 73, except alpha-Azido-omega-(propionic acid) 24(ethylene glycol) was used as the starting material. Upon collecting purified sample and drying on rotovap, 5.4 mg (71.0% yield) of a spectroscopically pure (90.4% AUC at 254 nm) purple solid was obtained. MS (ESI) calculated for C₄₀₂H₆₄₁N₃₅O₁₇₀ m/z 8679.3, found 1241.2 (M/7+H)⁺.

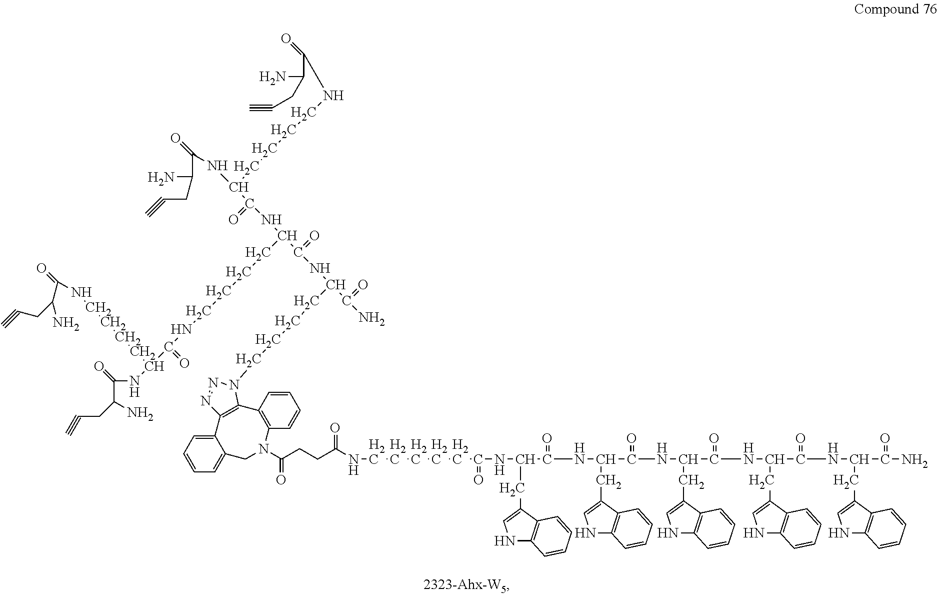 [SMILES: C#CCC(N)C(=O)NCCCCC(NC(=O)C(N)CC#C)C(=O)NCCCCC(NC(=O)C(CCCCNC(=O)C(N)CC#C)NC(=O)C(N)CC#C)C(=O)NC(CCCCn1nnc2c1-c1ccccc1N(C(=O)CCC(=O)NCCCCCC(=O)NC(Cc1c[nH]c3ccccc13)C(=O)NC(Cc1c[nH]c3ccccc13)C(=O)NC(Cc1c[nH]c3ccccc13)C(=O)NC(Cc1c[nH]c3ccccc13)C(=O)NC(Cc1c[nH]c3ccccc13)C(N)=O)Cc1ccccc1-2)C(N)=O]

Compound 76, referred to as {propargyl}₄K₂K{Lys(N₃)}-DBCO-Ahx-W₅ or 2323-Ahx-W₅, was synthesized by reacting 28.4 mg of Compound 62 (0.02 mmol, 1.2 eq) dissolved in dry DMSO and 23 mg of {propargyl}₄K₂K{Lys(N₃)}, which was prepared by solid phase peptide synthesis, (0.024 mmol, 1 eq) as a 100 mg/mL solution in dry DMSO was added. The reaction mixture was stirred overnight at room temperature. HPLC indicated the reaction was complete. Compound 76 was purified on a preparatory HPLC system using a gradient of 25-55% acetonitrile/H₂O (0.05% TFA) over 12 minutes on an Agilent Prep-C18 column, 30×100 mm, 5 μm. The product eluted at ˜4.5 minutes and the resulting fractions were collected, frozen and then lyophilized to obtain 32 mg (80.0% yield) of a spectroscopically pure (99.4% AUC at 254 nm) white powder. MS (ESI) calculated for C₁₂₄H₁₄₆N₂₈O₁₆ m/z 2283.2, found 1142.8 (M/2+H)⁺.

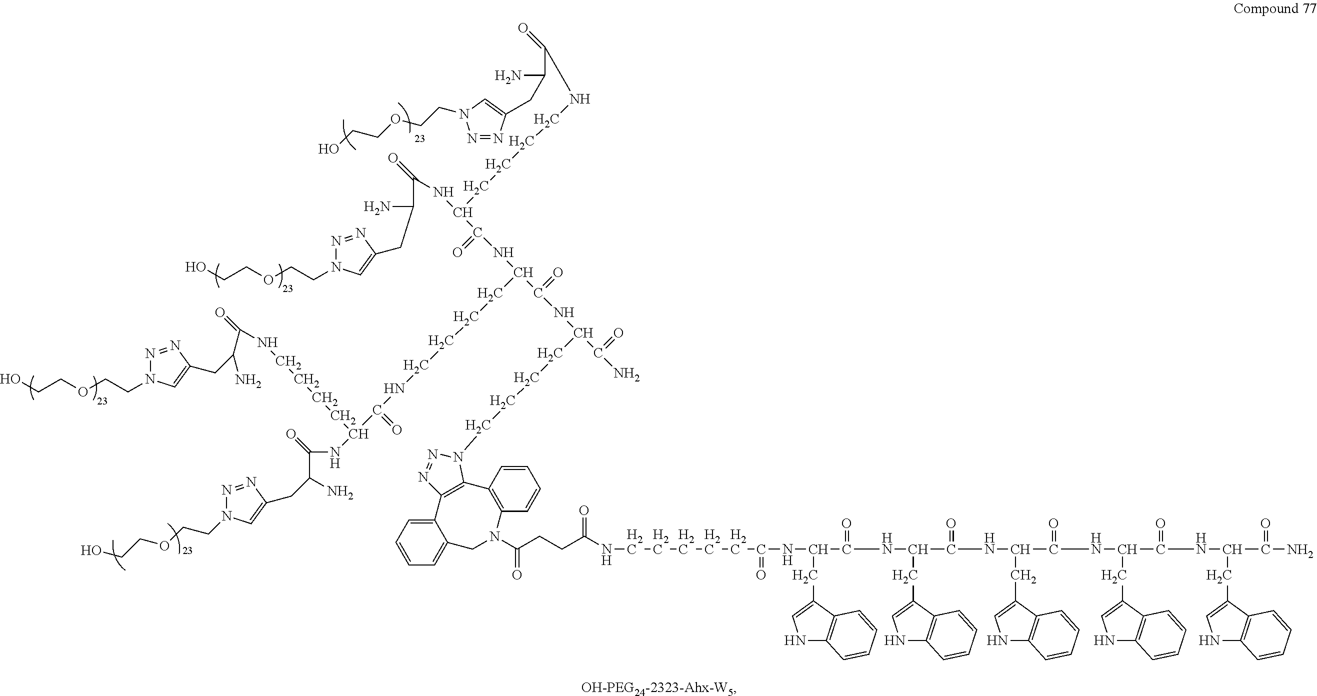

Compound 77, referred to as {OH-PEG2₄}4-{propargyl}₄K₂K{Lys(N₃)}-DBCO-Ahx-W₅ or OH-PEG₂₄-2323-Ahx-W₅, was synthesized by reacting 3.2 mg Compound 76 (0.0014 mmol, 1 eq) with 6.1 mg of alpha-Azido-omega-hydroxy 24(ethylene glycol) (0.0056 mmol, 4 eq) in 0.063 mL H₂O and 0.126 mL DMSO. To the reaction mixture, 1.1 mg Sodium Ascorbate (0.0056 mmol and 4 eq) was added. 1.4 mg Copper Sulfate Pentahydrate (0.0056 mmol, 4 eq) and 2.4 mg tris-hydroxypropyltriazolylmethylamine (0.0056 mmol, 4 eq) were combined in a separate vial, and then added to the reaction mixture. The reaction mixture was stirred overnight at room temperature. LC-MS indicated that the reaction was complete. Compound 77 was purified by dialysis with a Regenerated Cellulose membrane, MWCO: 2 kDa, with solvent changes of 1:1 H₂O/MeOH with 0.01% EDTA (2×), 1:1 H₂O/MeOH (1×), and MeOH (2×). Sample collected and dried on rotovap to obtain 7.1 mg (75.8% yield) of a spectroscopically impure (63.0% AUC at 254 nm) blue solid. MS (ESI) calculated for C₃₁₆H₅₃₄N₄₀O₁₁₂ m/z 6681.7, found 1337.8 (M/5+H)⁺.

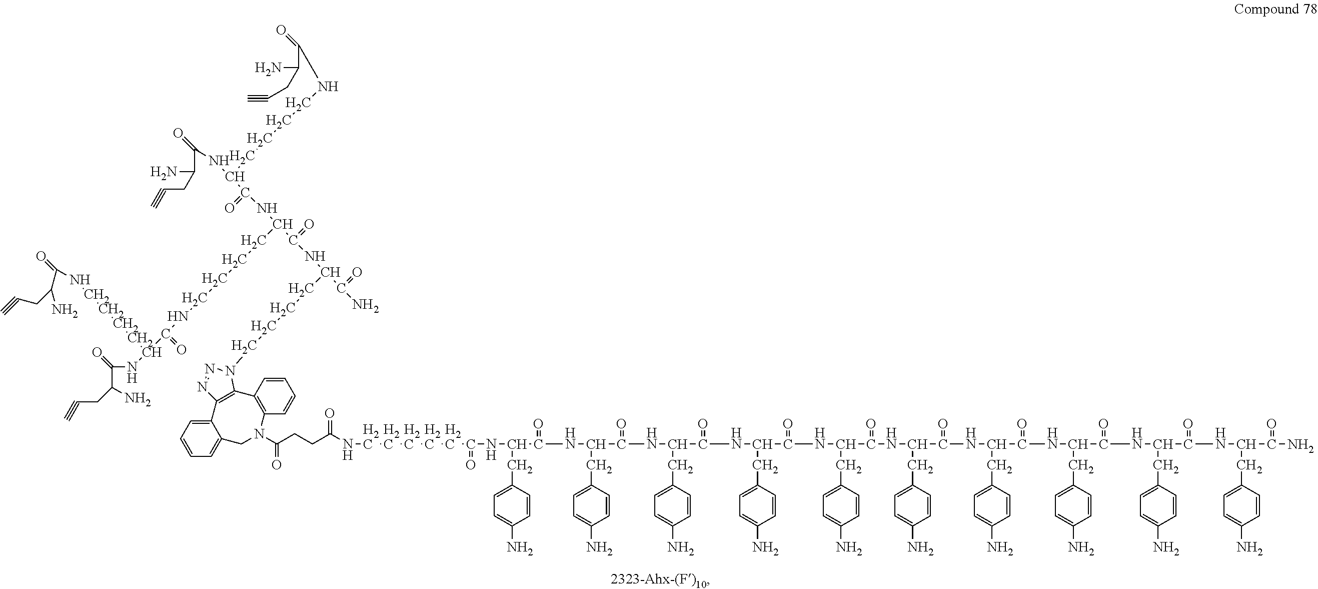 [SMILES: C#CCC(N)C(=O)NCCCCC(NC(=O)C(N)CC#C)C(=O)NCCCCC(NC(=O)C(CCCCNC(=O)C(N)CC#C)NC(=O)C(N)CC#C)C(=O)NC(CCCCn1nnc2c1-c1ccccc1N(C(=O)CCC(=O)NCCCCCC(=O)NC(Cc1ccc(N)cc1)C(=O)NC(Cc1ccc(N)cc1)C(=O)NC(Cc1ccc(N)cc1)C(=O)NC(Cc1ccc(N)cc1)C(=O)NC(Cc1ccc(N)cc1)C(=O)NC(Cc1ccc(N)cc1)C(=O)NC(Cc1ccc(N)cc1)C(=O)NC(Cc1ccc(N)cc1)C(=O)NC(Cc1ccc(N)cc1)C(=O)NC(Cc1ccc(N)cc1)C(N)=O)Cc1ccccc1-2)C(N)=O]

Compound 78, referred to as {propargyl}₄K₂K{Lys(N₃)}-DBCO-Ahx-(F′)₁₀ or 2323-Ahx-(F′)₁₀, was synthesized by reacting 88.6 mg of Compound 58 (0.04 mmol, 1.1 eq) and 41 mg of {propargyl}₄K₂K{Lys(N₃)}, which was prepared by solid phase peptide synthesis, (0.043 mmol, 1 eq) in 2.0 mL dry DMSO. The reaction mixture was stirred overnight at room temperature. HPLC indicated the reaction was complete and a resulted in a spectroscopically pure (96.1% AUC at 254 nm) colorless solution. MS (ESI) calculated for C₁₅₉H₁₉₆N₃₈O₂₁ m/z 2973.5, found 992.3 (M/3+H)⁺.

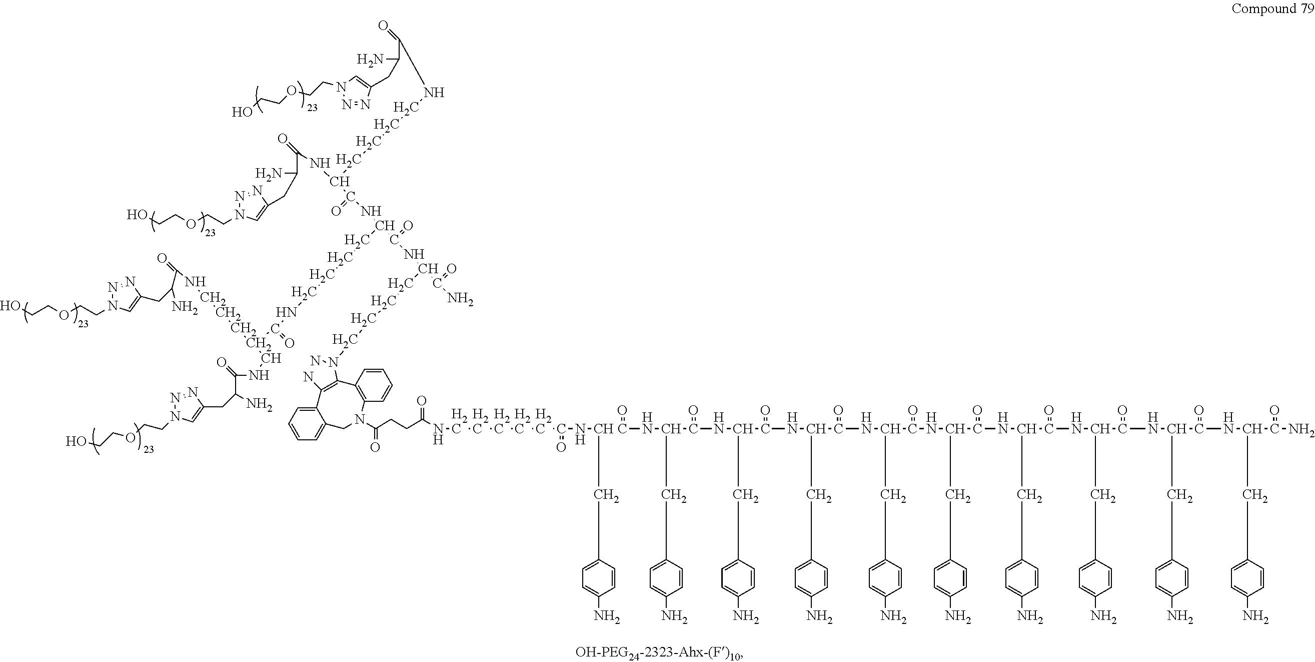

Compound 79, referred to as {OH-PEG₂₄}4-{propargyl}₄K₂K{Lys(N₃)}-DBCO-Ahx-(F′)₁₀, or OH-PEG₂₄-2323-Ahx-(F′)₁₀ was synthesized by reacting 4.0 mg Compound 79 (0.0013 mmol, 1 eq) with 5.9 mg of alpha-Azido-omega-hydroxy 24(ethylene glycol) (0.0054 mmol, 4 eq) in 0.050 mL H₂O and 0.099 mL DMSO. To the reaction mixture, 1.1 mg Sodium Ascorbate (0.0054 mmol, 4 eq) was added. 1.3 mg Copper Sulfate Pentahydrate (0.0054 mmol, 4 eq) and 2.3 mg tris-hydroxypropyltriazolylmethylamine (0.0054 mmol, 4 eq) were combined in a separate vial, and then added to the reaction mixture. The reaction mixture was stirred overnight at room temperature. LC-MS indicated that the reaction was complete. Compound 79 was purified by dialysis with a Regenerated Cellulose membrane, MWCO: 2 kDa, with solvent changes of 1:1 H₂O/MeOH with 0.01% EDTA (2×), 1:1 H₂O/MeOH (1×), and MeOH (2×). Sample collected and dried on rotovap to obtain 6.4 mg (64.6% yield) of a spectroscopically pure (94.1% AUC at 254 nm) green solid. MS (ESI) calculated for C₃₅₁H₅₈₄N₅₀O₁₁₇ m/z 7372.1, found 1230.2 (M/6+H)⁺.

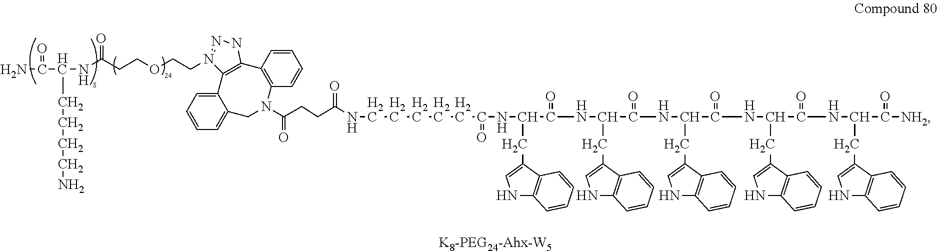

Compound 80, referred to as K₈-PEG24-(N3-DBCO)-Ahx-W₅ or K₈—PEG24-Ahx-W₅, was synthesized by first suspending K₈-PEG24-N3, which was prepared by solid phase peptide synthesis, in DMSO at 20 mg/mL and Compound 62 in DMSO at 100 mg/mL and then combining in a reaction vessel at a molar ratio of about 1.1 moles of H for every 1.0 moles of S-B. The reaction was performed at room temperature and determined to be complete after the S-B fragment was fully converted to S-B-H. This resulted in a spectroscopically pure (90.2% AUC at 254 nm) white solution. MS (ESI) calculated for C₁₇₉H₂₇₅N₃₃O₄₁ m/z 3543.0, found 1182.4 (M/3+H)⁺.

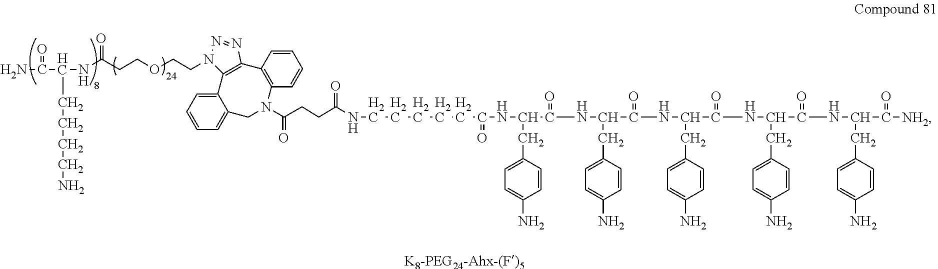

Compound 81, referred to as K₈-PEG₂₄-(N₃-DBCO)-Ahx-(F′)₅ or K₈-PEG₂₄-Ahx-(F′)₅, was synthesized and purified using the same procedure as Compound 80, except Compound 57 was used as the starting material. This resulted in a spectroscopically pure (86.8% AUC at 254 nm) white solution. MS (ESI) calculated for C₁₆₉H₂₇₅N₃₃O₄₁ m/z 3423.0, found 1142.4 (M/3+H)⁺.

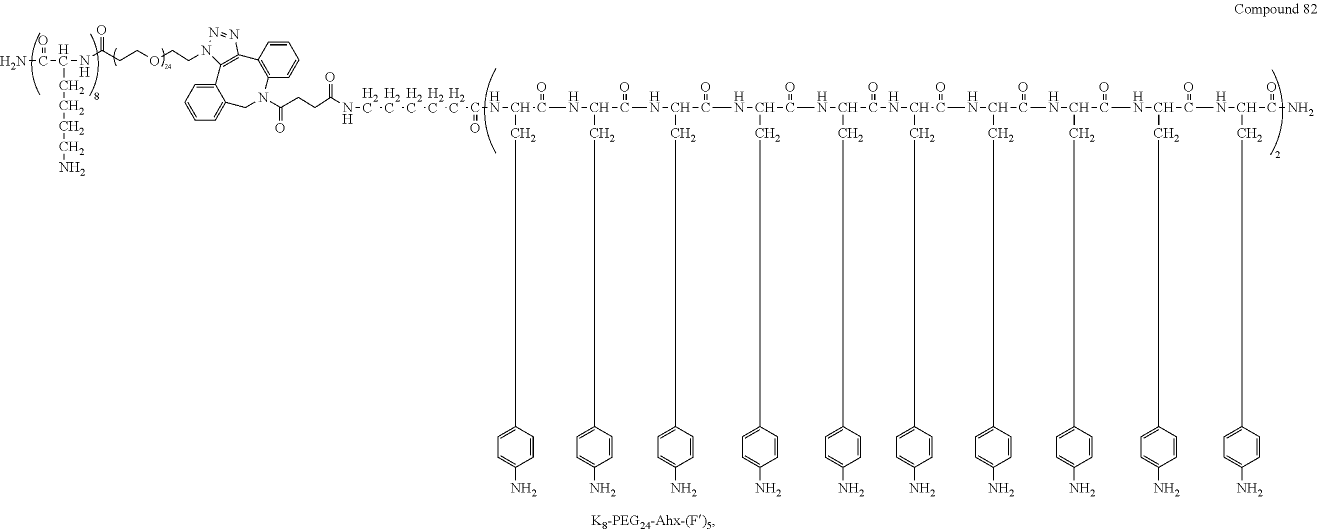

Compound 82, referred to as K₈-PEG₂₄-(N₃-DBCO)-Ahx-(F′)₂₀ or K₈-PEG₂₄-Ahx-(F′)₂₀, was synthesized and purified using the same procedure as Compound 80, except Compound 59 was used as the starting material. This resulted in a spectroscopically pure (89.2% AUC at 254 nm) light brown solution. MS (ESI) calculated for C₃₀₄H₄₂₅N₆₃O₅₆ m/z 5854.2, found 1171.2 (M/5+H)⁺.

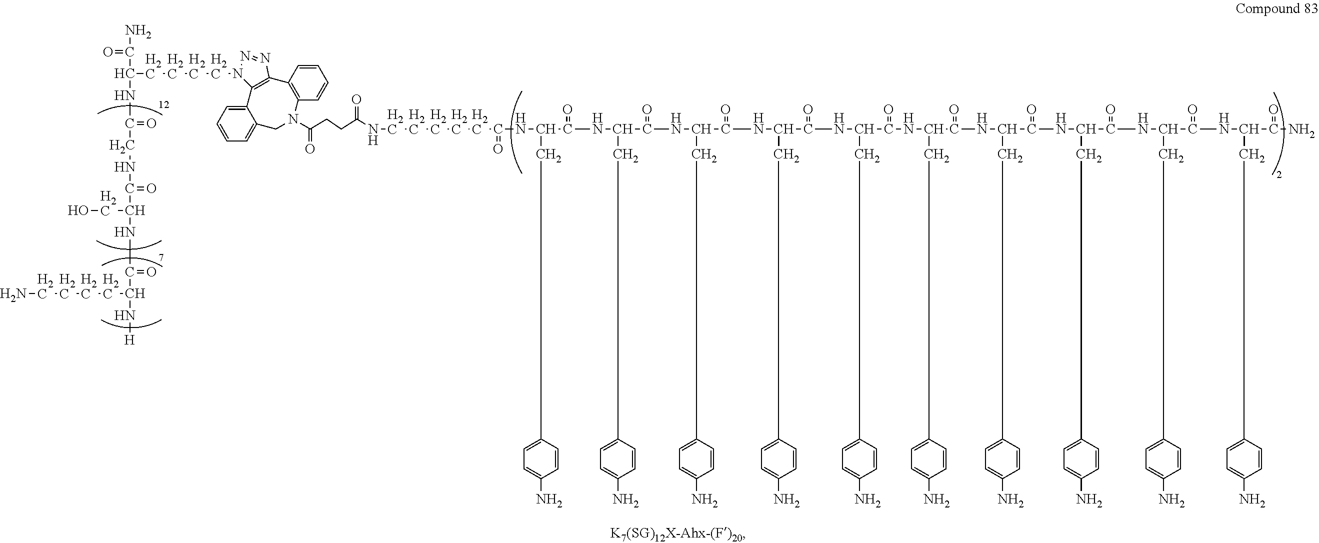

Compound 83, referred to as K₇(SG)₁₂X—(N₃-DBCO)-Ahx-(F′)₂₀ or K₇(SG)₁₂X -Ahx-(F′)₂₀, was synthesized and purified using the same procedure as Compound 80, except K₇(SG)₁₂X-N₃, which was prepared by solid phase peptide synthesis, and Compound 59 were used as the starting materials. This resulted in a spectroscopically pure (76.5% AUC at 254 nm) light brown solution. MS (ESI) calculated for C₃₁₃H₄₂₀N₈₆O₆₇ m/z 6455.2, found 1292.8 (M/5+H)⁺. Note: X=azido-lysine.

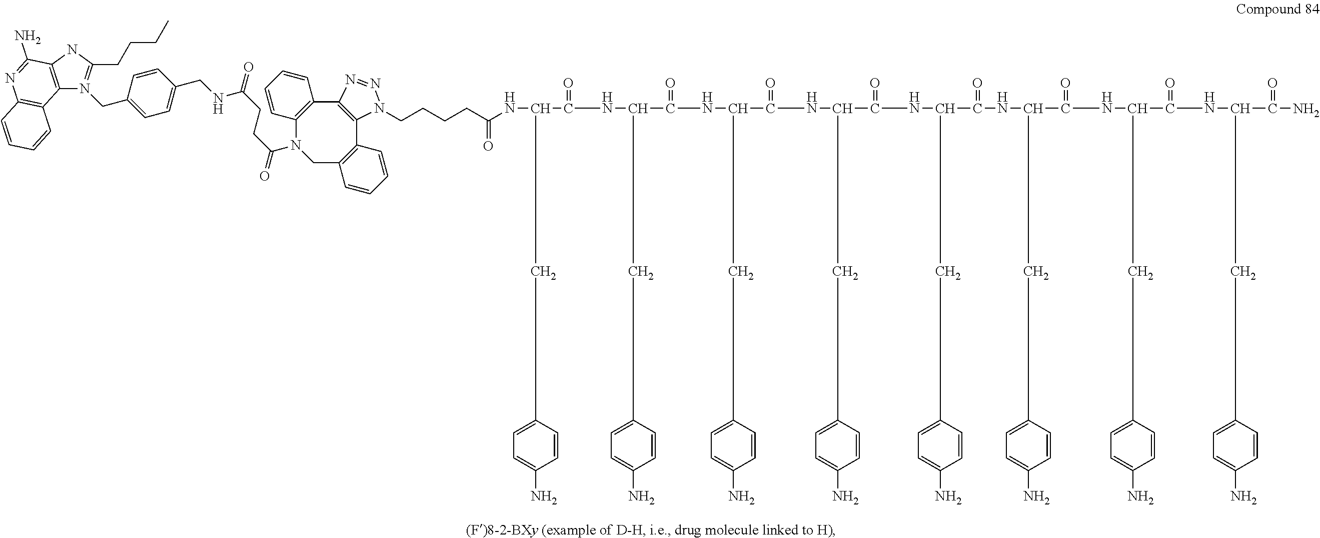

Compound 84, referred to as (F′)₈-(N₃-DBCO)-2BXy or (F′)₈-2BXy, was synthesized and purified using the same procedure as Compound 80, except (F′)₈—N₃, which was prepared by solid phase peptide synthesis, and DBCO-2BXy were used as the starting materials. This resulted in a spectroscopically pure (84.8% AUC at 254 nm) off-white solution. MS (ESI) calculated for C₁₁₈H₁₂₈N₂₆O₁₁ m/z 2085.0, found 1043.6 (M/2+H)⁺. F

Compound 84, referred to as (F′)₈-(N₃-DBCO)-2BXy or (F′)₈-2BXy, was synthesized and purified using the same procedure as Compound 80, except (F′)₈—N₃, which was prepared by solid phase peptide synthesis, and DBCO-2BXy were used as the starting materials. This resulted in a spectroscopically pure (84.8% AUC at 254 nm) off-white solution. MS (ESI) calculated for C₁₁₈H₁₂₈N₂₆O₁₁ m/z 2085.0, found 1043.6 (M/2+H)⁺. F

Compound 85, referred to as CpG-Ahx-W₅. CpG-based agonists of TLR-9 carry net charge at physiologic pH and can therefore function as the surface stabilizing group (S). To enable site-selective attachment of CpG to hydrophobic polymers or oligomers (S) a CpG sequence comprising an azide was prepared. Briefly, azide-modified CpG ODN 1826 of formula /5AzideN//iSp9/G*G*T*C*C**A**GA TC TTC*C*T*G*A*C*G*T*T was custom synthesized by Integrated DNA Technologies (IDT, Coralville, IA, USA), wherein !5AzideN//iSp9/ is an azide-terminated PEG3 spacer linked at the 5′-0H of the DNA sequence G*G*T*C*C*A*T*G*A*C*G*T*T*C*C*T*G*A*C*G*T*T with a phosphorothioate backbone. As a non-limiting example, DBCO-Ahx-W₅ (Compound 62) was reacted with the azide bearing CpG sequence in a DMSO/PBS solution at room temperature to generate an amphiphilic block copolymer, wherein S-B and H were linked together through a triazole group. The reaction was monitored by gel permeation chromatography, which showed that the CpG was completely converted to the product after 16 hours at room temperature. The resulting amphiphile (Compound 85) was not turbid (OD at 490 nm<0.04) and formed stable nanoparticle micelles, ˜20 nm, diameter, when resuspended in PBS at 0.5 mg/mL.

Compound 86, referred to as HPMA-Ahx-F′10. A pHPMA (HPMA polymer) with a molecular weight of˜7,000 g/mol terminated with an azide group was prepared as previously described. Briefly, Azide-pHPMA was synthesized via the RAFT polymerization of HPMA using CTA-Azide as a chain transfer agent and ACVA-Azide as an initiator in tert-butanol/N,N-dimethylacetamide at 70° C. for 6.5 hours. The resulting polymer (Azide-pHPMA-DTB) was purified by precipitating in acetone/diethyl ether and dried to yield light pink solid. The CTA (DTB) was removed by reacting the resulting solid in tert-butanol at 80° C. for 2 hours with 20-fold molar excess ACVA. The resulting capped polymer Azide-pHPMA was analyzed by GPC-MALS to confirm that the molecular weight was approximately 7,000 g/mol (or “Daltons” or “Da”), corresponding a polymer with approximately 50 monomeric units. As a non-limiting example, DBCO-Ahx-F′ ₁₀ (Compound 58) was reacted with azide-pHPMA at room temperature to generate an amphiphilic block copolymer, wherein S and H were linked together through a triazole group. The reaction was monitored by gel permeation chromatography, which showed that the CpG was completely converted to the product after 16 hours at room temperature. The resulting amphiphile (Compound 86) copolymer was not turbid (OD at 490 nm<0.04) and formed stable nanoparticle micelles, ˜20 nm, diameter, when resuspended in PBS at 0.5 mg/mL.

### Example 2. Synthesis and Characterization of Amphiphiles of Formula S-[B]-[U]-H-[D]

A combinatorial library of different S-B-U-H compositions was prepared by reacting different compositions of hydrophobic blocks (H) bearing an alkyne linker precursor U2, with different compositions of S-B-U1 bearing an azide. Each of the precursors were first suspended in DMSO at greater than 20 mg/mL DMSO and then combined in a reaction vessel at a molar ratio of about 1.05 moles of U2-H for every 1.0 moles of S-B-U1. The reactions were performed at room temperature and determined to be complete after the S-B-U1 fragment was fully converted to S-B-U-H. This reaction scheme was used to prepare different compositions of amphiphiles, which were characterized for the capacity to form nanoparticle micelles in aqueous buffer, PBS pH 7.4, at a concentration of 0.5 mg/mL. The results of these studies are summarized below according to the chemical composition and architecture of the amphiphilic block copolymers.

### Linear Amphiphiles Comprising Peptides

A series of linear amphiphilic block copolymers of formula S-B-U-H, wherein the solubilizing block and spacer (B) both comprise peptides, i.e., poly(lysine) and poly(serine-co-glycine), respectively, with varying hydrophobic polymer composition were evaluated for particle size and stability by dynamic light scattering. The results show that nanoparticle micellization is highly dependent on the net charge of, with S-B-H with net charge of +8 and comprising hydrophobic polymers with up to 20 hydrophobic amino acids based on Phe(NH2), i.e., phenylalanine-amine, sometimes abbreviated F′ forming stable nanoparticle micelles, whereas those with +4 net charge were found to aggregate (Table 3).

TABLE 3
Peptide-based linear amphiphiles.

| | | | | Size |
|---|---|---|---|---|
| | Cmpd | Net | | (diameter, |
| Composition (S-B-U-H), U = (Lys(N3-DBCO)) | # | charge | MW | nm) |
| KKK-SGSGSGSGSGSGSGSGSGSGSGSG-(Lys(N3-DBCO))-Ahx-(F′)5 | 87 | 4 | 3660.44 | 1013 |
| KKK-SGSGSGSGSGSGSGSGSGSGSGSG-(Lys(N3-DBCO))-Ahx-(F′)10 | 88 | 4 | 4470.64 | 3839 |
| KKK-SGSGSGSGSGSGSGSGSGSGSGSG-(Lys(N3-DBCO))-Ahx-(F′)20 | 89 | 4 | 6092.46 | 1521 |
| KKK-SGSGSGSGSGSGSGSGSGSGSGSG-(Lys(N3-DBCO))-Bis | 90 | 4 | 6419.44 | 2683 |
| (Ahx-F′10)2 | | | | |
| KKKKKKK-SGSGSGSGSGSGSGSGSGSGSGSG-(Lys(N3-DBCO))-Ahx-(F′)5 | 91 | 8 | 4173.14 | 477 |
| KKKKKKK-SGSGSGSGSGSGSGSGSGSGSGSG-(Lys(N3-DBCO))-Ahx-(F′)10 | 92 | 8 | 4983.34 | 16 |
| KKKKKKK-SGSGSGSGSGSGSGSGSGSGSGSG-(Lys(N3-DBCO))-Ahx-(F′)20 | 93 | 8 | 6605.16 | 32 |
| KKKKKKK-SGSGSGSGSGSGSGSGSGSGSGSG-(Lys(N3-DBCO))- | 94 | 8 | 6932.14 | 56 |
| (Ahx-(F′)10)2 | | | | |

Note:
single letter abbreviations for amino acids are used in the above table.

### Linear Amphiphiles Comprising Peptides and PEG

A series of linear amphiphiles of formula S-B-U-H, wherein the charged molecule solubilizing block (S) comprises peptides and the spacer (B) comprises a hydrophilic polymer, i.e., PEG, with varying hydrophobic polymer composition were evaluated for particle size and stability by dynamic light scattering.

Similar to the results observed with amphiphiles comprising peptide-based spacers, nanoparticle micellization was highly dependent on the net charge (see Table 4).

TABLE 4
Amphiphiles with linear architecture comprising peptide-based solubilizing blocks
and hydrophilic PEG polymer-based spacers.

| | Cmpd | Net | | Size |
|---|---|---|---|---|
| Composition (S-B-U-H), U = (Azide-DBCO) | # | charge | MW | (diameter, nm) |
| KK-PEG4-(azide-DBCO)-Ahx-(F′)5 | 94 | 2 | 1776.4 | 2427 |
| KK-PEG4-(azide-DBCO)-Ahx-(F′)10 | 95 | 2 | 2586.6 | 1112 |
| KK-PEG4-(azide-DBCO)-Ahx-(F′)20 | 96 | 2 | 4208.42 | 3618 |
| KK-PEG4-(azide-DBCO)-(Ahx-(F′)10)2 | 97 | 2 | 4535.4 | 2650 |
| KK-PEG24-(azide-DBCO)-Ahx-(F′)5 | 98 | 2 | 2656.76 | 467 |
| KK-PEG24-(azide-DBCO)-Ahx-(F′)10 | 99 | 2 | 3466.96 | 24 |
| KK-PEG24-(azide-DBCO)-Ahx-(F′)20 | 100 | 2 | 5088.78 | 2205 |
| KK-PEG24-(azide-DBCO)-(Ahx-(F′)10)2 | 101 | 2 | 5415.76 | 1492 |
| KKKK-PEG4-(azide-DBCO)-Ahx-(F′)5 | 102 | 4 | 2032.1 | 2069 |
| KKKK-PEG4-(azide-DBCO)-Ahx-(F′)10 | 103 | 4 | 2842.3 | 14 |
| KKKK-PEG4-(azide-DBCO)-Ahx-(F′)20 | 104 | 4 | 4464.12 | 2890 |
| KKKK-PEG4-(azide-DBCO)-(Ahx-(F′)10)2 | 105 | 4 | 4791.1 | 2121 |
| KKKK-PEG24-(azide-DBCO)-Ahx-(F′)5 | 106 | 4 | 2913.1 | 1392 |
| KKKK-PEG24-(azide-DBCO)-Ahx-(F′)10 | 107 | 4 | 3723.3 | 22 |
| KKKK-PEG24-(azide-DBCO)-Ahx-(F′)20 | 108 | 4 | 5345.12 | 103 |
| KKKK-PEG24-(azide-DBCO)-(Ahx-(F′)10)2 | 109 | 4 | 5672.1 | 30 |
| KKKKKKKK-PEG4-(azide-DBCO)-Ahx-(F′)5 | 110 | 8 | 2544.69 | 1181 |
| KKKKKKKK-PEG4-(azide-DBCO)-Ahx-(F′)10 | 111 | 8 | 3354.89 | 11 |
| KKKKKKKK-PEG4-(azide-DBCO)-Ahx-(F′)20 | 112 | 8 | 4976.71 | 19 |
| KKKKKKKK-PEG4-(azide-DBCO)-(Ahx-(F′)10)2 | 113 | 8 | 5303.69 | 17 |
| KKKKKKKK-PEG24-(azide-DBCO)-Ahx-(F′)5 | 114 | 8 | 3425.79 | 49 |
| KKKKKKKK-PEG24-(azide-DBCO)-Ahx-(F′)10 | 115 | 8 | 4235.99 | 16 |
| KKKKKKKK-PEG24-(azide-DBCO)-Ahx-(F′)20 | 116 | 8 | 5857.81 | 21 |
| KKKKKKKK-PEG24-(azide-DBCO)-(Ahx-(F′)10)2 | 117 | 8 | 6184.79 | 26 |

Note:
single letter abbreviations for amino acids are used in the above table; and, oligo(lysine) sequences in the above table were linked to the PEG spacer through the N-terminus and are terminated with an amide.

Amphiphiles with Dendron-Based S Blocks Having Cone Architecture

A series of cone-shaped amphiphiles of formula S-B-U-H, wherein the solubilizing block (S) comprises peptides of dendritic structure and the spacer (B) comprises a hydrophilic polymer, i.e., PEG, with varying hydrophobic polymer composition were evaluated for particle size and stability by dynamic light scattering. The amphiphiles with cone architecture comprising peptide-based S blocks generally required high net charge to induce nanoparticle micellization (see Table 5).

TABLE 5
Amphiphiles with cone architecture comprising peptide-based solubilizing blocks

| | | | | Size |
|---|---|---|---|---|
| Composition (C-B-U-H), U = (Lys(N3-DBCO)) | Cmpd # | Net charge | MW | (diameter, nm) |
| K2K-PEG4-Lys(N3-DBCO)-Ahx-(F′)5 | 119 | 4 | 2033.3 | 1685 |
| K2K-PEG4-Lys(N3-DBCO)-Ahx-(F′)10 | 120 | 4 | 2843.5 | 53 |
| K2K-PEG4-Lys(N3-DBCO)-Ahx-(F′)20 | 121 | 4 | 4465.32 | 2038 |
| K2K-PEG4-Lys(N3-DBCO)-(Ahx-(F′)10)2 | 122 | 4 | 4792.3 | 2000 |
| K2K-PEG24-Lys(N3-DBCO)-Ahx-(F′)5 | 123 | 4 | 2913.09 | 3 |
| K2K-PEG24-Lys(N3-DBCO)-Ahx-(F′)10 | 124 | 4 | 3723.29 | 24 |
| K2K-PEG24-Lys(N3-DBCO)-Ahx-(F′)20 | 125 | 4 | 5345.11 | 5590 |
| K2K-PEG24-Lys(N3-DBCO)-(Ahx-(F′)10)2 | 126 | 4 | 5672.09 | 2000 |
| K4K2K-PEG4-Lys(N3-DBCO)-Ahx-(F′)5 | 127 | 8 | 2544.7 | 532 |
| K4K2K-PEG4-Lys(N3-DBCO)-Ahx-(F′)10 | 128 | 8 | 3354.9 | 10 |
| K4K2K-PEG4-Lys(N3-DBCO)-Ahx-(F′)20 | 129 | 8 | 4976.72 | 20 |
| K4K2K-PEG4-Lys(N3-DBCO)-(Ahx- | 130 | 8 | 5303.7 | 67 |
| (F)10)2 | | | | |
| K4K2K-PEG24-Lys(N3-DBCO)-Ahx-(F′)5 | 131 | 8 | 3425.77 | 892 |
| K4K2K-PEG24-Lys(N3-DBCO)-Ahx-(F′)10 | 132 | 8 | 4235.97 | 17 |
| K4K2K-PEG24-Lys(N3-DBCO)-Ahx-(F′)20 | 133 | 8 | 5857.79 | 32 |
| K4K2K-PEG24-Lys(N3-DBCO)-(Ahx- | 134 | 8 | 6184.77 | 2000 |
| (F)10)2 | | | | |

Note: single letter abbreviations for amino acids are used in the above table; and,

K2K and K4K2K are lysine dendrons comprising 3 and 7 lysines, respectively. For clarity, the structure of K2K (linked to a spacer, B) is shown here for clarity:

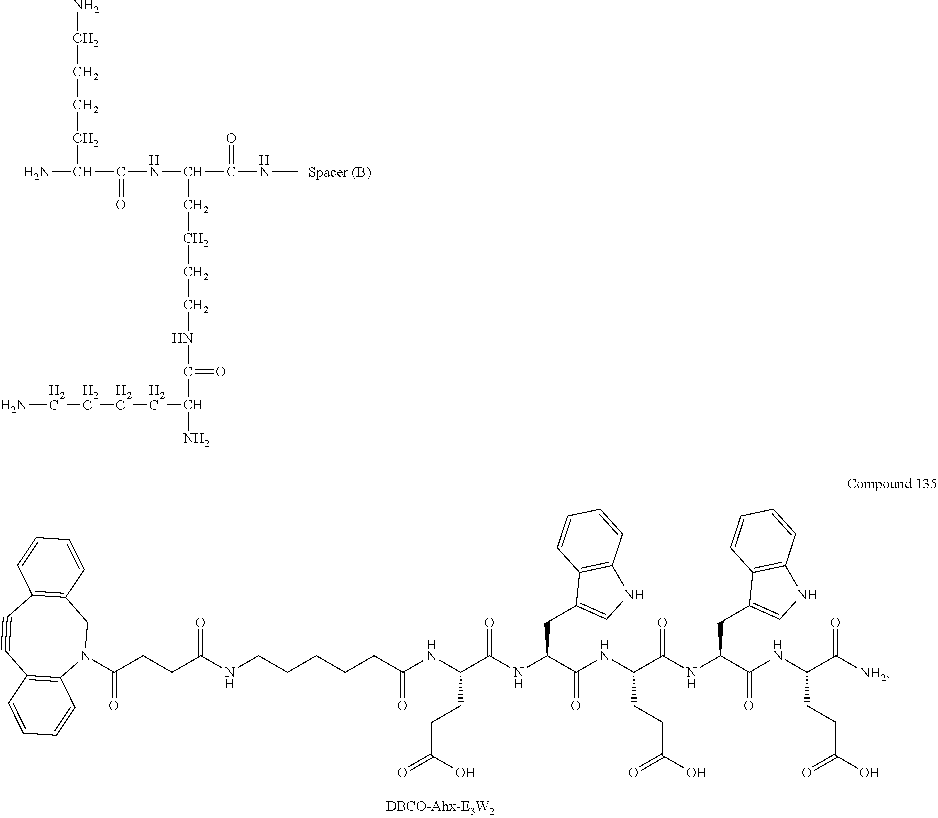 [SMILES: CNC(=O)C(CCCCNC(=O)C(N)CCCCN)NC(=O)C(N)CCCCN ; NC(=O)[C@H](CCC(=O)O)NC(=O)[C@H](Cc1c[nH]c2ccccc12)NC(=O)[C@H](CCC(=O)O)NC(=O)[C@H](Cc1c[nH]c2ccccc12)NC(=O)[C@H](CCC(=O)O)NC(=O)CCCCCNC(=O)CCC(=O)N1Cc2ccccc2C#Cc2ccccc21]

Compound 135, referred to as DBCO-Ahx-E₃W₂ or DBCO-Ahx-(Glu)₃(Trp)₂, was synthesized from a 6-aminohexanoic-Glu-Trp-Glu-Trp-Glu-NH₂ or Ahx-E₃W₂ precursor prepared by solid-phase peptide synthesis. 105 mg of Ahx-E₃W₂(0.12 mmol, 1 eq) and 65.8 uL triethylamine (TEA) (0.47 mmol, 4 eq) were added to 525 uL anhydrous DMF and stirred at room temperature under ambient air for 5 minutes. 52.2 mg of DBCO-NHS ester (Scottsdale, Arizona, USA) (0.13 mmol, 1.1 eq) was then added while stirring vigorously and reacted for 1 hour. The reaction progress was monitored by HPLC (AUC 254 nm). After 1 hour the reaction was complete, and the reaction was quenched by adding amino-PEG₂₄-OH (San Diego, California, USA) (1 eq) and stirring for 1 hour. The reaction mixture was added dropwise to 5 mL of 0.2 M HCl to precipitate an off-white powder which was collected by centrifuging the solution at 4000 g at 4° C. for 5 minutes. The HCl solution was discarded and Compound 135 was collected as a solid off-white pellet. The off-white solid was re-suspended in 525 uL DMF and added dropwise to 5 mL of DI water and spun at 3000 g at 4° C. for 5 minutes; the DI water solution was discarded, and Compound 135 was collected as a solid pellet. This process was repeated and then the solid was collected and dried under vacuum to yield 117 mg of a spectroscopically pure (>95% AUC at 220 nm) white powder. MS (ESI) calculated for C₆₂H₆₈N₁₀O₁₄ m/z 1176.5, found 588.8 (M/2+H)⁺.

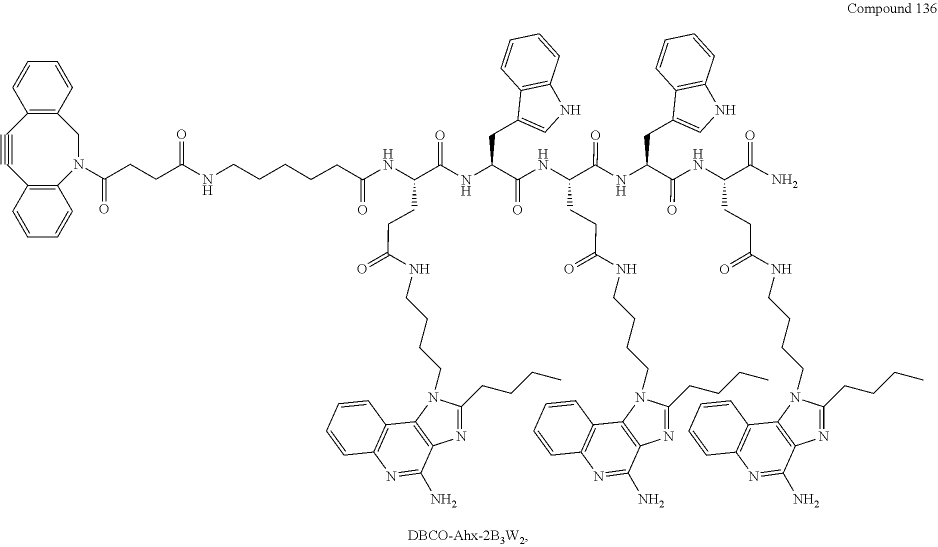 [SMILES: CCCCc1nc2c(N)nc3ccccc3c2n1CCCCNC(=O)CC[C@H](NC(=O)[C@H](Cc1c[nH]c2ccccc12)NC(=O)[C@H](CCC(=O)NCCCCn1c(CCCC)nc2c(N)nc3ccccc3c21)NC(=O)[C@H](Cc1c[nH]c2ccccc12)NC(=O)[C@H](CCC(=O)NCCCCn1c(CCCC)nc2c(N)nc3ccccc3c21)NC(=O)CCCCCNC(=O)CCC(=O)N1Cc2ccccc2CCc2ccccc21)C(N)=O]

Compound 136, referred to as DBCO-Ahx-2B₃W₂, DBCO-Ahx-E(2B)₃W₂ or DBCO-Ahx-Glu(2B)₃(Trp)₂ was synthesized by reacting Compound 135 and Compound 33 in the presence of HATU. 142.2 uL Triethylamine (TEA) (1.02 mmol, 12 eq) was diluted in 1 mL anhydrous DMF, and 100 mg of Compound 135 (0.09 mmol, 1 eq) and 103 mg of Compound 33 (0.33 mmol, 3.9 eq) were added while stirring vigorously until fully dissolved. The reaction mixture was cooled to 4° C. by immersion in an ice bath for 5 minutes, then 106.6 mg of HATU (0.28 mmol, 3.3 eq) was added. The reaction mixture stirred vigorously for 1 hour at 4° C. and the reaction progress was monitored by HPLC (AUC 254 nm). The resulting product was purified on a preparatory HPLC system using a gradient of 30-45% acetonitrile/H2O (0.05% TFA) over 12 minutes on an Agilent Prep C-18 column, 30×100 mm, 5 μm. The product eluted at ˜40% acetonitrile and the resulting fractions were combined, frozen and lyophilized to give 99.1 mg (60% yield) of a spectroscopically pure white powder (>95% AUC at 220 nm). MS (ESI) Calculated for C₁₁₆H₁₃₇N₂₅O₁₁ m/z 2056.1, found 685.4 (M/3+H)⁺.

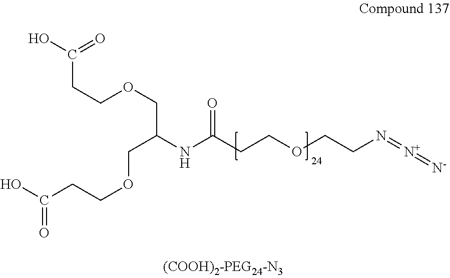

Compound 137, referred to as (COOH)₂—PEG₂₄-N₃ was synthesized by reacting 2.8 g of N₃—P₂₄-NHS ester (2.2 mmol, 1 eq) and 0.57 g of 2-Amino-1,3-bis(carboxylethoxy)propane HCl salt (2.1 mmol, 0.95 eq) dissolved in 30 mL anhydrous DCM. Triethylamine (3 mL, 22.1 mmol, 10 eq) was added to the reaction mixture. The reaction was stirred at room temperature for 3 hours until HPLC indicated the reaction was complete. The reaction solvent was removed under vacuum and the reaction mixture was redissolved in 1:1 DMSO/H2O w/0.05% TFA. The product was purified by flash C18 chromatography on a 12 g Biotage SNAP C18 column using a 2-step gradient: 0% acetonitrile in H2O (0.05% TFA) over 3 column volumes (CVs), followed by 0-60% acetonitrile in H₂O (0.05% TFA) over 20 CVs. The product eluted at ˜25% acetonitrile and the resulting fractions were collected and the solvent removed under vacuum to yield 2.0 g (65.2% yield) of a spectroscopically pure (>97% AUC at 220 nm) white oil. MS (ESI) calculated for C₆₀H₁₁₆N₄0₃₁ m/z 1388.8, found 1412.6 (M+Na+H)⁺.

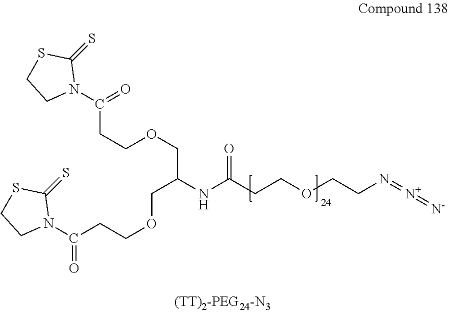

Compound 138, referred to as (TT)₂-PEG₂₄-N₃ was synthesized by reacting 2.0 g of Compound 137 (1.5 mmol, 1 eq) and 1.2 g of HATU (3.2 mmol, 2.2 eq) in 24 mL of DCM. The mixture was cooled on ice for 5 min and 1.6 mL of triethylamine (11.7 mmol, 8 eq) was added. The mixture was stirred on ice for 5 min and 0.45 g of thizoline-2-thio (TT) (3.8 mmol, 2.6 eq) was added. The reaction mixture stirred at room temperature for 2 h until HPLC indicated the reaction was complete. The product was purified by flash chromatography on a 100 g Biotage Safar SilicaD column over a 2-step gradient: 0% methanol in DCM over 3 column volumes (CVs), followed by 0-8% methanol in DCM over 20 CVs. The product eluted at ˜5% methanol and the resulting fractions were collected and the solvent removed to obtain 2.0 g (85.3% yield) of a spectroscopically pure (96.1% AUC at 220 nm) yellow oil. MS (ESI) Calculated for C₆₆H₁₂₂N₉O₂₉S₄ m/z 1590.7, found 782.3 ((M-N3)/2)⁺.

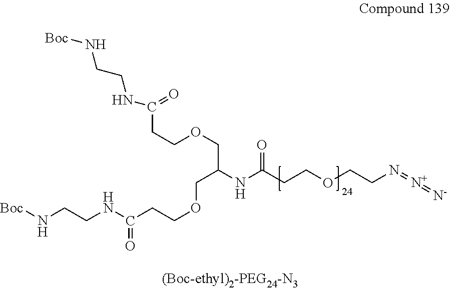

Compound 139, referred to as (Boc-ethyl)₂-PEG₂₄-N₃ was synthesized by reacting 347 mg of Compound 138 (0.2 mmol, 1 eq) and 83 mg of N-boc-ethylenediamine (0.5 mmol, 2.4 eq) in 3.5 mL of DCM. Triethylamine (73 uL, 0.5 mmol, 2.4 eq) was added and the reaction mixture was stirred at room temperature for 1 h until HPLC indicated the reaction was complete. The solvent was removed under vacuum and the product was dissolved in DMSO and purified on a preparatory HPLC system using a gradient of 27-57% acetonitrile/H₂O (0.05% TFA) over 12 minutes on an Agilent Prep-C18 column, 30×100 mm, 5 μm. The product eluted at ˜7.2 minutes and the resulting fractions were collected, frozen and then lyophilized to obtain 189 mg (51.8% yield) of a spectroscopically pure (94.7% AUC at 220 nm) white solid. MS (ESI) Calculated for C₇₄H₁₄₄N₈O₃₃ m/z 1672.98, found 1574.6 (M-Boc+H)⁺

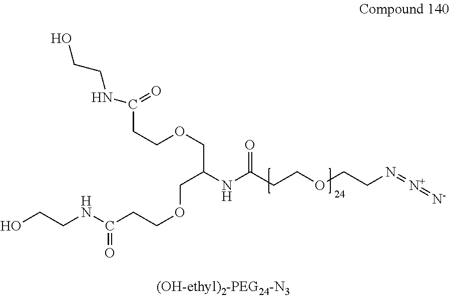

Compound 140, referred to as (OH-ethyl)₂-PEG₂₄-N₃ was synthesized following the same procedure as Compound 139, except ethanolamine was used instead of N-boc-ethylenediamine. Compound 140 was purified on a preparatory HPLC system using a gradient of 15-45% acetonitrile/H₂O (0.05% TFA) over 12 minutes on an Agilent Prep-C18 column, 30×100 mm, 5 μm. The product eluted at ˜7.1 minutes and the resulting fractions were collected, frozen and then lyophilized to obtain an 81.2% yield of a spectroscopically pure (98.3% AUC at 220 nm) white solid. MS (ESI) Calculated for C₆₄H₁₂₆N₆O₃₁ m/z 1474.9, found 11476.6 (M+H)⁺.

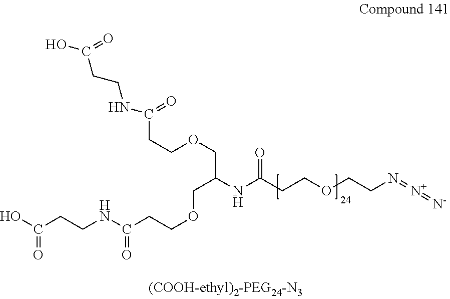

Compound 141, referred to as (COOH-ethyl)₂-PEG₂₄-N₃ was synthesized following the same procedure as Compound 139, except beta-alanine was used instead of N-boc-ethylenediamine and MeOH was used as the solvent. Compound 141 was purified on a preparatory HPLC system using a gradient of 13-43% acetonitrile/H₂O (0.05% TFA) over 2 minutes on an Agilent Prep-C18 column, 30×100 mm, 5 μm. The product eluted at ˜9.2 minutes and the resulting fractions were collected, frozen and then lyophilized to obtain a 37.2% yield of as spectroscopically pure (90.0% AUC at 220 nm) white solid. MS (ESI) Calculated for C₆₆H₁₂₆N₈O₃₃ m/z 1530.8, found 1533.6 (M+H)⁺

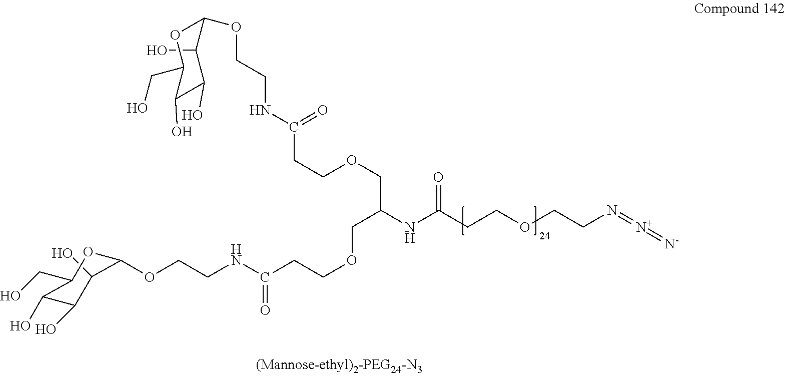

Compound 142, referred to as (Mannose-ethyl)₂-PEG₂₄-N₃ was synthesized following the same procedure as Compound 139, except 2-Aminoethyl-a-Mannopyranoside (Broadpharm (San Diego, CA)) was used instead of N-boc-ethylenediamine and DMSO was used as the solvent. Compound 142 was purified on a preparatory HPLC system using a gradient of 15-45% acetonitrile/H₂O (0.05% TFA) over 12 minutes on an Agilent Prep-C18 column, 30×100 mm, 5 μm. The product eluted at ˜7.0 minutes and the resulting fractions were collected, frozen and then lyophilized to obtain a 70.0% yield of a spectroscopically pure (98.2% AUC at 220 nm) white solid. MS (ESI) Calculated for C₇₆H₁₄₆N₆O₄₁ m/z 1799.0.

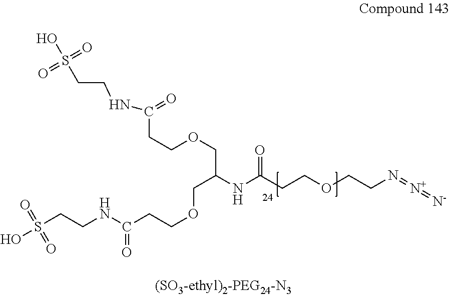

Compound 143, referred to as (SO₃-ethyl)₂-PEG24-N3 was synthesized following the same procedure as Compound 139, except taurine was used instead of N-boc-ethylenediamine and 2:1 DMSO/PBS was used as the solvent. Compound 143 was purified on a preparatory HPLC system using a gradient of 15-40% acetonitrile/H₂O (0.05% TFA) over 12 minutes on an Agilent Prep-C18 column, 30×100 mm, 5 μm. The product eluted at ˜6.7 minutes and the resulting fractions were collected, frozen and then lyophilized to obtain a 47.1% yield of a spectroscopically pure (>99% AUC at 220 nm) white solid. MS (ESI) Calculated for C₆₄H₁₂₆N₆O₃₅S₂ m/z 1602.8, found 802.4 (M/2+H)⁺

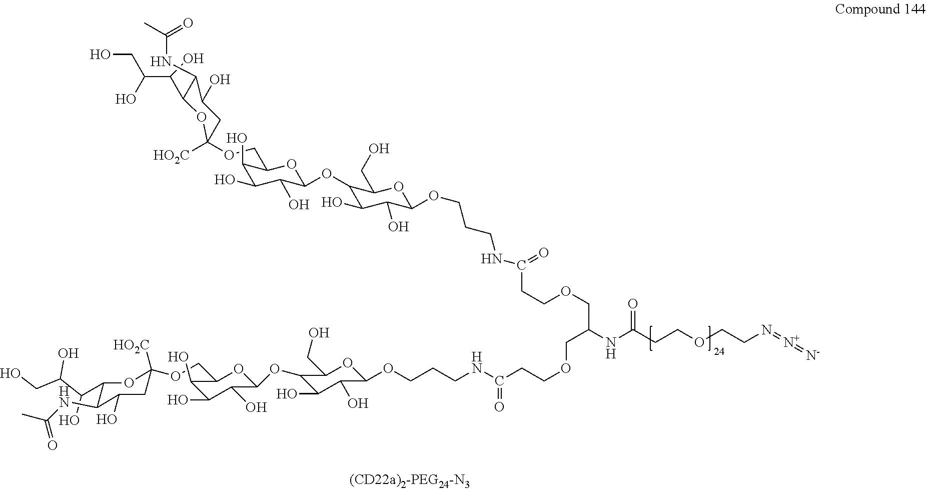

Compound 144, referred to as (CD22a)₂-PEG₂₄-N₃ was synthesized following the same procedure as Compound 139, except CD22a amine was used instead of N-boc-ethylenediamine and DMSO was used as the solvent. Compound 144 was purified on a preparatory HPLC system using a gradient of 15-35% acetonitrile/H₂O (0.05% TFA) over 12 minutes on an Agilent Prep-C18 column, 30×100 mm, 5 μm. The product eluted at ˜8.1 minutes and the resulting fractions were collected, frozen and then lyophilized to obtain a 68.8% yield of a spectroscopically pure (98.3% AUC at 220 nm) white solid. MS (ESI) Calculated for C₁₁₂H₂₀₄N₈O₆₇ m/z 2733.3, found 1367.8 (M/2+H)⁺

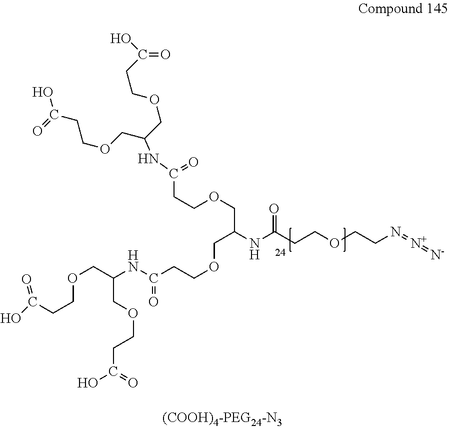

Compound 145, referred to as (COOH)₄—PEG₂₄-N₃ was synthesized by reacting 1.5 g of Compound 138 (0.9 mmol, 1 eq) and 0.4 g of 2-Amino-1,3-bis(carboxylethoxy)propane HCl salt (1.4 mmol, 1.6 eq) dissolved in 35 mL of anhydrous DCM. Triethylamine (2.5 mL, 18.3 mmol, 19 eq) was added to the reaction mixture. The reaction was stirred at room temperature for 4 hours until HPLC indicated the reaction was complete. Compound 145 was not purified. MS (ESI) Calculated for C₇₈H₁₄₆N₆O₄₁ m/z 1823.0, found 912.4 (M/2+H)⁺.

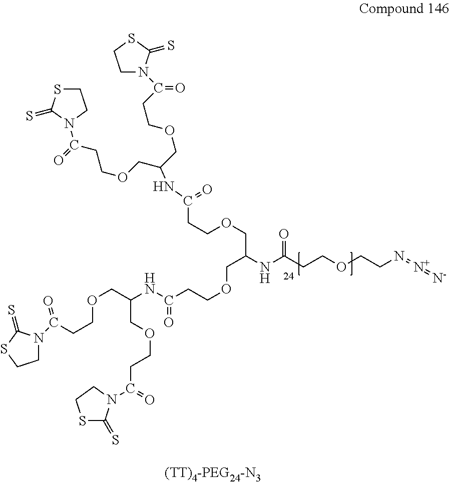

Compound 146, referred to as (TT)₄-PEG₂₄-N₃ was synthesized by reacting 1.5 g of Compound 145 (0.8 mmol, 1 eq) and 1.4 g of HATU (3.7 mmol, 4.4 eq) in 3 mL of DCM. Triethylamine (1.9 mL, 13.5 mmol, 16 eq) was added and the reaction mixture was stirred for 5 min. Thiazoline-2-thio (TT) (0.5 g, 4.4 mmol, 5 eq) was added and the reaction mixture was stirred at room temperature for 3 h until HPLC indicated the reaction was complete. The product was purified by flash chromatography on a 100 g Biotage Safar SilicaD column over a 2-step gradient: 0% methanol in DCM over 3 column volumes (CVs), followed by 0-8% methanol in DCM over 20 CVs. The product eluted at ˜5% methanol and the resulting fractions were collected and the solvent removed to obtain 0.8 g (42% yield) of a yellow oil (70% AUC at 220 nm). MS (ESI) Calculated for C₉₀H₁₅₈N₁₀O₃₇S₈ m/z 2228.8, found 962.8 (M/2)⁺

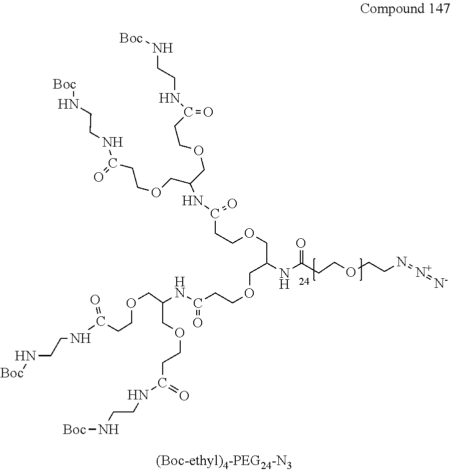

Compound 147, referred to as (Boc-ethyl)₄-PEG₂₄-N₃ was synthesized by reacting 246 mg of Compound 146 (0.1 mmol, 1 eq) and 84 mg of N-boc-ethylenediamine (0.5 mmol, 4.8 eq) in 2.6 mL of DCM. Triethylamine (74 uL, 0.5 mmol, 4.8 eq) was added and the reaction mixture was stirred at room temperature for 1 h until HPLC indicated the reaction was complete. The solvent was removed under vacuum and the product was dissolved in DMSO and purified on a preparatory HPLC system using a gradient of 32-60% acetonitrile/H₂O (0.05% TFA) over 12 minutes on an Agilent Prep-C18 column, 50×100 mm, 5 μm. The product eluted at ˜7.1 minutes and the resulting fractions were collected, frozen and then lyophilized to obtain 100.4 mg (40.0% yield) of a spectroscopically pure (94.9% AUC at 220 nm) white solid. MS (ESI) Calculated for C₁₀₆H₂₀₂N₁₄O₄₅ m/z 2392.8, found 1197.0 (M/2+H)⁺.

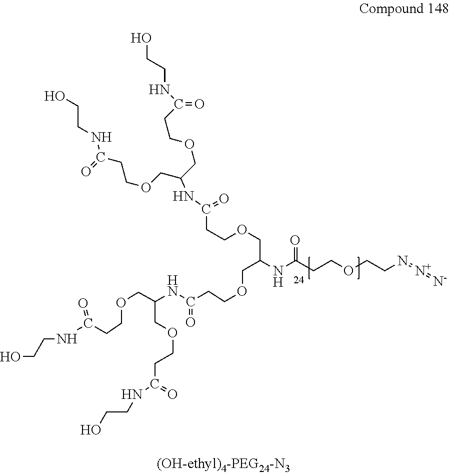

Compound 148, referred to as (OH-ethyl)₄-PEG₂₄-N₃ was synthesized following the same procedure as Compound 147, except ethanolamine was used instead of N-boc-ethylenediamine. Compound 148 was purified on a preparatory HPLC system using a gradient of 17-37% acetonitrile/H₂O (0.05% TFA) over 12 minutes on an Agilent Prep-C18 column, 30×100 mm, 5 μm. The product eluted at ˜7.0 minutes and the resulting fractions were collected, frozen and then lyophilized to obtain a 43.6% yield of a spectroscopically pure (97.2% AUC at 220 nm) white solid. MS (ESI) Calculated for C₈₆H₁₆₆N₁₀O₄₁ m/z 1995.1, found 998.6 (M/2+H)⁺

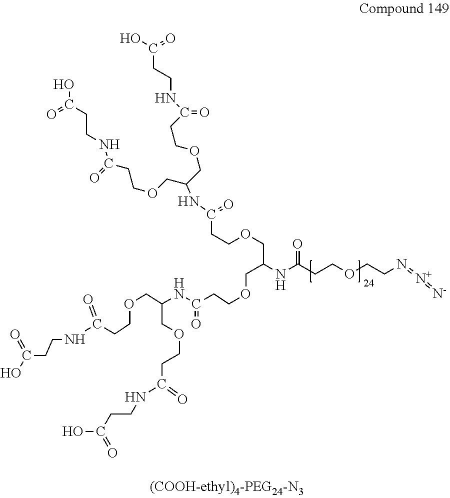

Compound 149, referred to as (COOH-ethyl)₄-PEG₂₄-N₃ was synthesized following the same procedure as Compound 147, except beta-alanine was used instead of N-boc-ethylenediamine and MeOH was used as the solvent. Compound 149 was purified on a preparatory HPLC system using a gradient of 19-39% acetonitrile/H₂O (0.05% TFA) over 2 minutes on an Agilent Prep-C18 column, 30×100 mm, 5 μm. The product eluted at ˜6.8 minutes and the resulting fractions were collected, frozen and then lyophilized to obtain a 34.3% yield of as spectroscopically pure (86.0% AUC at 220 nm) white solid. MS (ESI) Calculated for C₉₀H₁₆₆N₁₀O₄₅ m/z 2107.1, found 1054.7 (M/2+H)⁺

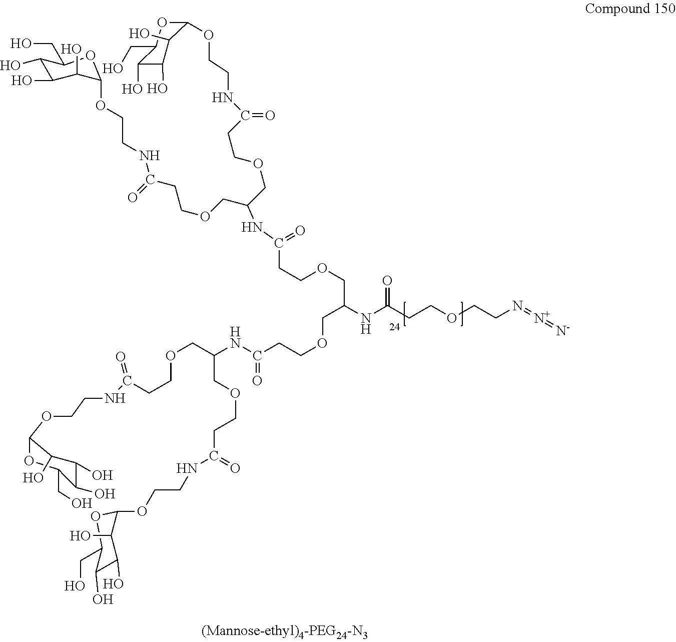

Compound 150, referred to as (Mannose-ethyl)₄-PEG₂₄-N₃ was synthesized following the same procedure as Compound 147, except 2-Aminoethyl-a-Mannopyranoside (Broadpharm (San Diego, CA)) was used instead of N-boc-ethylenediamine and DMSO was used as the solvent. Compound 150 was purified on a preparatory HPLC system using a gradient of 15-35% acetonitrile/H₂O (0.05% TFA) over 12 minutes on an Agilent Prep-C18 column, 30×100 mm, 5 μm. The product eluted at ˜7.3 minutes and the resulting fractions were collected, frozen and then lyophilized to obtain a 35.6% yield of a spectroscopically pure (98.5% AUC at 220 nm) white solid. MS (ESI) Calculated for C₁₃₃H₂₅₂N₁₀O₆₁ m/z 2965.7, found 1322.9 (M/2)⁺.

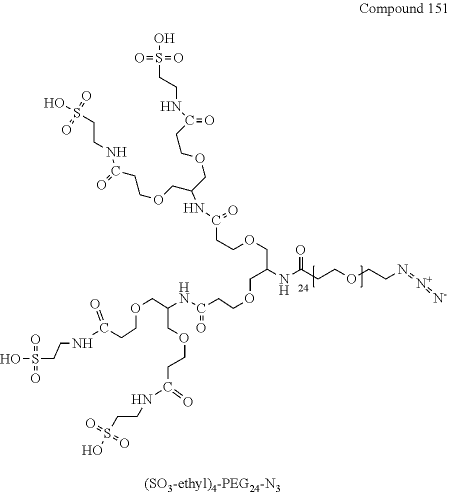

Compound 151, referred to as (SO₃-ethyl)₄-PEG₂₄-N₃ was synthesized following the same procedure as Compound 147, except taurine was used instead of N-boc-ethylenediamine and 2:1 DMSO/PBS was used as the solvent. Compound 151 was purified on a preparatory HPLC system using a gradient of 5-45% acetonitrile/H₂O (0.05% TFA) over 12 minutes on an Agilent Prep-C18 column, 30×100 mm, 5 μm. The product eluted at ˜7.4 minutes and the resulting fractions were collected, frozen and then lyophilized to obtain a 30.2% yield of a spectroscopically pure (96.9% AUC at 220 nm) white solid. MS (ESI) Calculated for C₈₆H₁₆₆N₁₀O₄₉S₄ m/z 2251.0, found 1126.7 (M/2)⁺

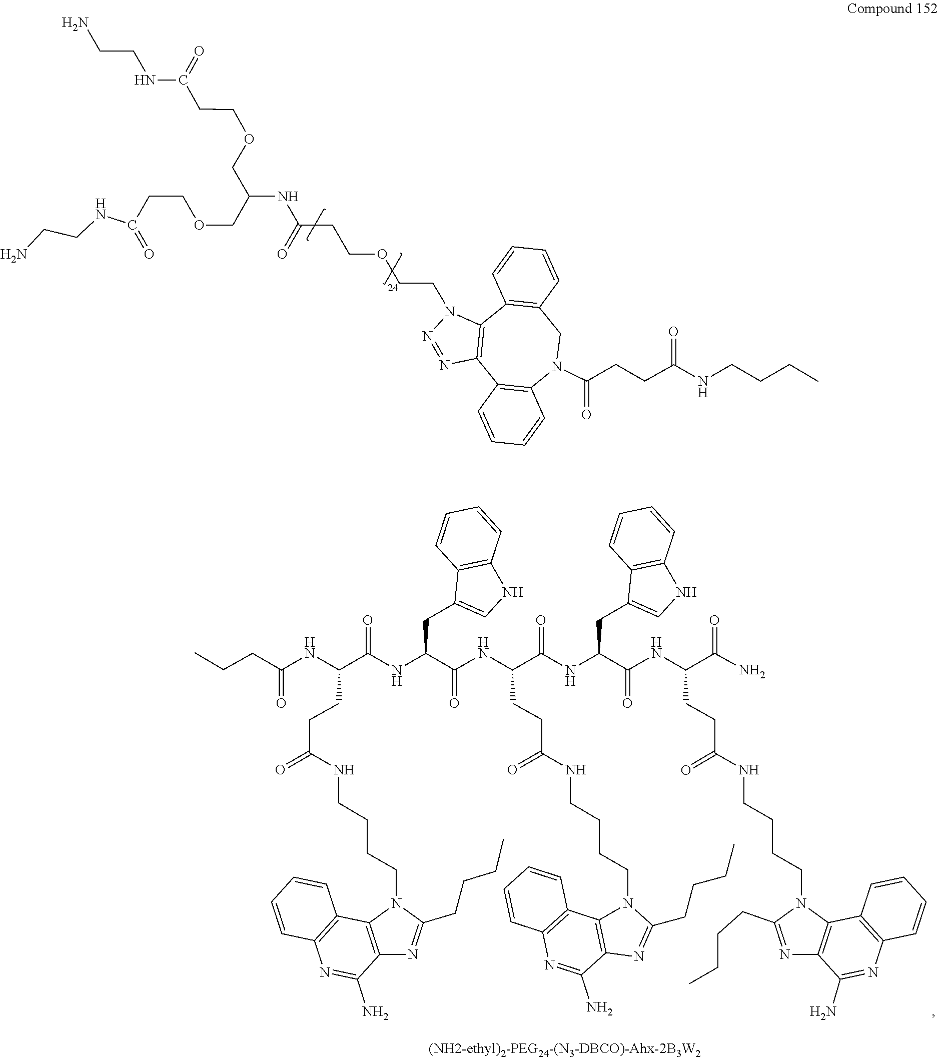

Compound 152, referred to (NH₂-ethyl)₂-PEG₂₄-(N₃-DBCO)-Ahx-2B₃W₂ was synthesized by reacting Compound 139 (0.001 mmol, 1.0 eq) dissolved in anhydrous DMSO and Compound 136 (0.001 mmol, 1.05 eq) as a 50 mM solution in anhydrous DMSO. The reaction mixture was stirred overnight at room temperature. The DMSO solvent was then removed. The boc protected intermediate was deprotected by resuspending in 100% trifluoroacetic acid (TFA) (200 uL) for 1 minute and the TFA was then removed. The remaining solution was washed twice with diethyl ether (200 uL). HPLC indicated the deprotection was complete and resulted in 2.1 mg (38.4% yield) of a spectroscopically pure (89.6% AUC at 220 nm) off-white solid. MS (ESI) calculated for C₁₈₀H₂₆₅N₃₃O₄₀ m/z 3531.3, found 1177.8 (M/3)⁺.

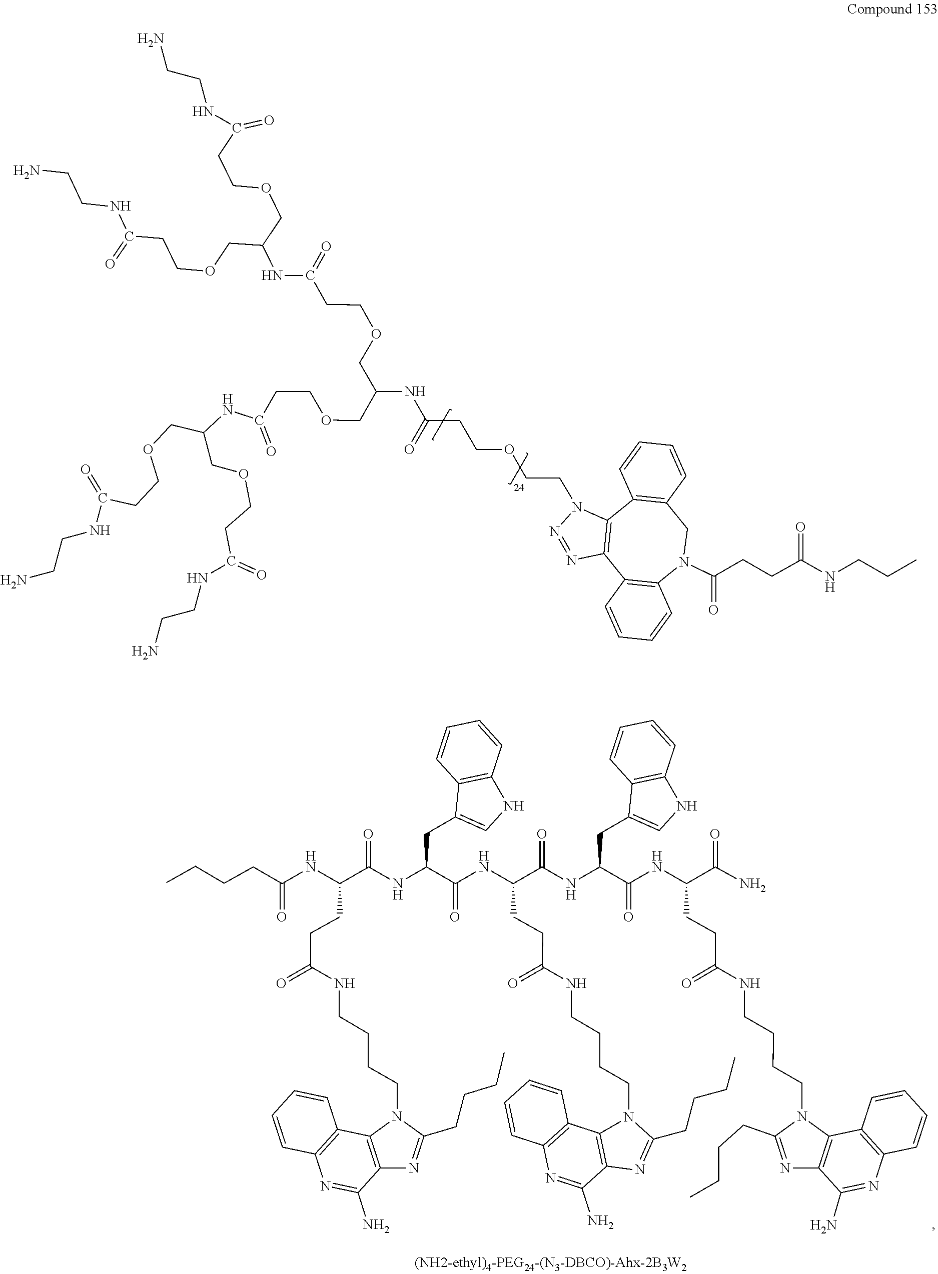

Compound 153, referred to (NH₂-ethyl)₄-PEG₂₄-(N₃-DBCO)-Ahx-2B₃W₂ was synthesized using the same procedure as Compound 152, except Compound 147 was used in place of Compound 139 and resulted in a spectroscopically pure (92.7% AUC at 220 nm) off-white solid. MS (ESI) calculated for C₂₀₂H₃₀₇N₃₉O₄₈ m/z 4047.3, found 1013.3 (M/4+H)⁺.

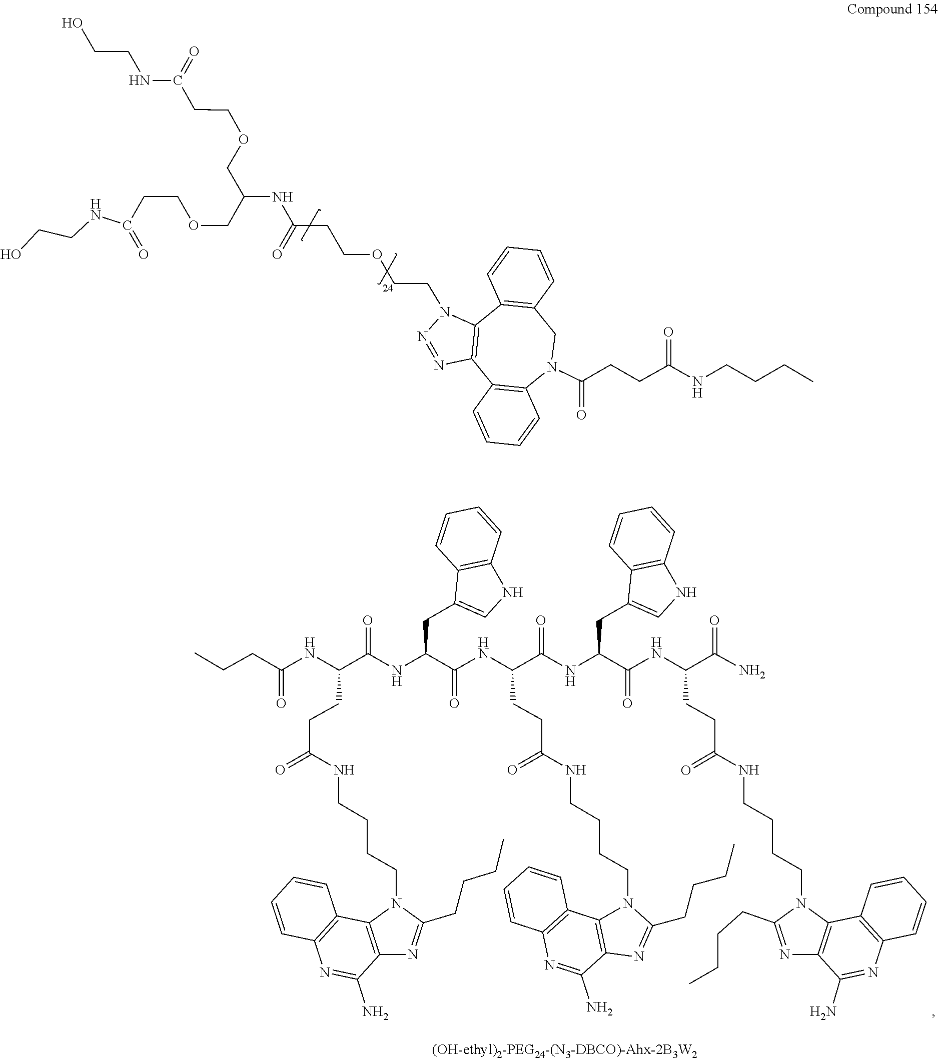

Compound 154, referred to (OH-ethyl)₂-PEG₂₄-(N₃-DBCO)-Ahx-2B₃W₂ was synthesized by reacting Compound 140 (0.001 mmol, 1.0 eq) with Compound 136 (0.001 mmol, 1.05 eq) in anhydrous DMSO for 16 hours at room temperature. HPLC was monitored to evaluate reaction progress and indicated complete conversion of Compound 140 to Compound 154, resulting in a spectroscopically pure (89.2% AUC at 220 nm) colorless solution. MS (ESI) calculated for C₁₈₀H₂₆₃N₃₁O₄₂ m/z 3530.9, found 1178.3 (M/3+H)⁺.

Compounds 155-164 were produced in a similar manner as that described for Compound 154. Table 6 provides a summary of the synthesis and characterization of compounds 155-164.

TABLE 6
Amphiphilic block copolymers of formula S-B-U-H-[D]
having cone architecture, i.e., dendron-based solubilizing
block (S) and linear spacer and hydrophobic block architecture.

| | (S-B-U1) | U2-H[D] | | | MS | Particle |
|---|---|---|---|---|---|---|
| Cmpd | Compound # | Compound # | | m/z | (ESI) | Size |
| # | Product S-B-U-H-[D] | | Charge | Theo. | m/n Found | (nm) |
| 155 | Compound 141 | Compound 136 | −2 | 3586.9 | 1197.0 | 11.9 |
| | (COOH-ethyl)₂₋ PEG₂₄-(N3- | | | | (M/3 + H)⁺ | |
| | DBCO)-Ahx-2B₃W₂ | | | | | |
| 156 | Compound 142 | Compound 136 | 0 | 1799.0 | 1286.6 | 12.5 |
| | (Mannose-ethyl)₂- PEG₂₄-(N3- | | | | (M/3 + H)⁺ | |
| | DBCO)-Ahx-2B₃W₂ | | | | | |
| 157 | Compound 143 | Compound 136 | −2 | 2658.9 | 1221.0 | 12.8 |
| | (SO3-ethyl)₂- PEG₂₄-(N3- | | | | (M/3 + H)⁺ | |
| | DBCO)-Ahx-2B₃W₂ | | | | | |
| 158 | Compound 144 | Compound 136 | −2 | 4789.4 | 1198.7 | 16.6 |
| | (CD22a)₂- PEG₂₄-(N3- | | | | (M/4 + H)⁺ | |
| | DBCO)-Ahx-2B₃W₂ | | | | | |
| 159 | Compound 148 | Compound 136 | 0 | 4051.2 | 1351.9 | 12.8 |
| | (OH-ethyl)₄- PEG₂₄-(N3- | | | | (M/3 + H)⁺ | |
| | DBCO)-Ahx-2B₃W₂ | | | | | |
| 160 | Compound 149 | Compound 136 | −4 | 4163.2 | 1389.2 | 21.9 |
| | (COOH-ethyl)₄- PEG₂₄-(N3- | | | | (M/3 + H)⁺ | |
| | DBCO)-Ahx-2B₃W₂ | | | | | |
| 161 | Compound 149 | Compound 62 | −4 | 3455.7 | 1153.1 | |
| | (COOH-ethyl)₄- PEG₂₄-(N3- | | | | (M/3 + H)⁺ | |
| | DBCO)-Ahx-WWWWW | | | | | |
| 162 | Compound 150 | Compound 136 | 0 | 4698.4 | 1176.3 | 11.8 |
| | (Mannose-ethyl)₄- PEG₂₄-(N3- | | | | (M/4 + H)⁺ | |
| | DBCO)-Ahx-2B₃W₂ | | | | | |
| 163 | Compound 150 | Compound 62 | 0 | 3990.9 | 1331.9 | 50.2 |
| | (Mannose-ethyl)₄- PEG₂₄-(N3- | | | | (M/3 + H)⁺ | |
| | DBCO)-Ahx-WWWWW | | | | | |
| 164 | Compound 151 | Compound 136 | −4 | 4307.1 | 1437.2 | 14.4 |

| | |
|---|---|
| (SO3-ethyl)₄- PEG₂₄-(N3- | (M/3 + H)⁺ |
| DBCO)-Ahx-2B₃W₂ | |

Net charge is the predicted net charge of the amphiphile in aqueous buffer at pH 7.4.

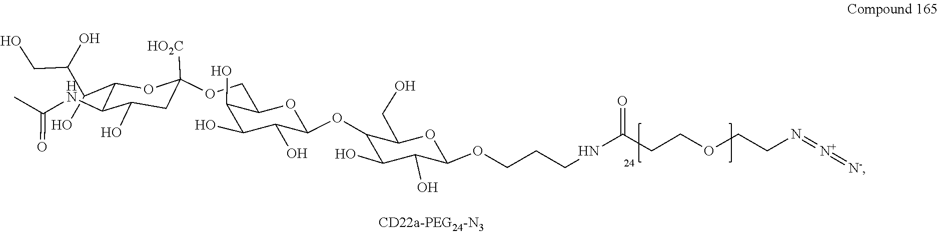

Compound 165, referred to CD22a-PEG₂₄-N₃ was synthesized by dissolving 74.8 mg of CD22a Amine (0.11 mmol, 1 eq) in 3.75 mL anhydrous DMSO. 49.9 uL TEA (0.36 mmol, 3.3 eq) was added and the solution was stirred at room temperature for five minutes. 165.1 mg of NHS-PEG₂₄-N₃ (0.13 mmol, 1.2 eq) was added to the reaction mixture and the reaction was stirred at room temperature for 1 hour when LC-MS indicated reaction was complete. The product was purified on a preparatory HPLC system using a gradient of 15-45% acetonitrile/H₂O (0.05% TFA) over 12 minutes on an Agilent Prep-C18 column, 30×100 mm, 5 μm. The product eluted at ˜6 minutes and the resulting fractions were collected, frozen and then lyophilized to obtain 167 mg (73.7% yield) of a spectroscopically pure (94.9% AUC at 220 nm) white solid. MS (ESI) Calculated for C₇₇H₁₄₅N₅O₄₄ m/z 1843.9, found 923.1 (M/2+H)⁺.

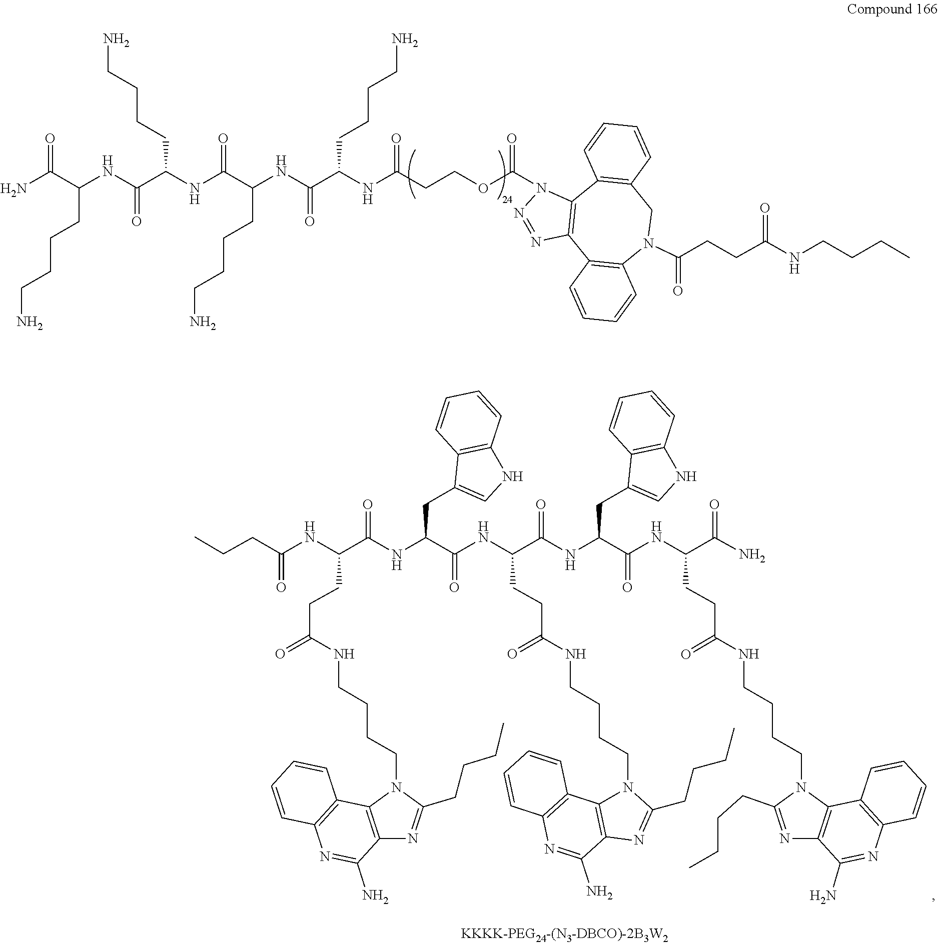

Compound 166, referred to KKKK-PEG₂₄-(N3-DBCO)-Ahx-2B3W₂ was synthesized using a facile click chemistry reaction by reacting 1 equivalent of Compound 136 with 1 equivalent of Azide-PEG₂₄-KKKK in DMSO at room temperature. The reaction progress was monitored by HPLC, which confirmed complete conversion of starting materials to Compound 166 after 16 hours. MS (ESI) Calculated for C₁₉₀H₂₈₃N₃₇O₄₁ m/z 3739.1, found 923.1 (M/2+H)⁺.

Similar reaction conditions were used to produce Compounds 167-177 summarized in Table 7.

TABLE 7
Amphiphiles of formula S-B-U-H-[D] having linear architecture.

| | | Starting U2-H[D] | | | | Particle |
|---|---|---|---|---|---|---|
| Cmpd | Starting (S-B-U1) | Compound # | Net | Theo. | Found | Size |
| # | Product (S-B-U-H-[D]) | | charge | MW | MW | (nm) |
| 167 | N₃-PEG₄-KKKKKKKK-NH₂ | Compound 136 | +8 | 3371.8 | Conforms | 9.4 |
| | KKKKKKKK-PEG4-(N3-DBCO)-Ahx-2B₃W₂ | | | | | |
| 168 | N₃-PEG₁₂-KKKKKKKK-NH₂ | Compound 136 | +8 | 3724.2 | Conforms | 74.2 |
| | KKKKKKKK-PEG₁₂-(N₃-DBCO)-Ahx-2B₃W₂ | | | | | |
| 169 | N₃-PEG₂₄-KKKKKKKK-NH₂ | Compound 136 | +8 | 4252.9 | Conforms | 27.7 |
| | KKKKKKKK-PEG₂₄-(N₃-DBCO)-Ahx-2B₃W₂ | | | | | |
| 170 | KKKKKKKKK-PEG₃₆-X | Compound 136 | +9 | 5037.8 | Conforms | 34.2 |
| | KKKKKKKKK-PEG₃₆-(N₃-DBCO)-Ahx-2B₃W₂ | | | | | |
| 171 | NH₂-PEG₍₅₀₀₀₎-N₃ | Compound 136 | +1 | 7056.1 | Conforms | 52.1 |
| | NH₂-PEG₍₅₀₀₀₎-(N₃-DBCO)-Ahx-2B₃W₂ | | | | | |
| 172 | N₃-PEG₂₄-KKKKKKKK-NH₂ | Compound 62 | +8 | 3545.2 | Conforms | 23.9 |
| | KKKKKKKK-PEG₂₄-(N₃-DBCO)-Ahx-WWWWW | | | | | |
| 173 | KKKKKKK- | Compound 136 | +8 | 4854.1 | Conforms | 11.3 |
| | SGSGSGSGSGSGSGSGSGSGSGSGX-NH₂ | | | | | |
| | KKKKKKK-SGSGSGSGSGSGSGSGSGSGSGSGX-(N₃-DBCO)- | | | | | |
| | Ahx-2B₃W₂ | | | | | |
| 174 | N₃-PEG₂₄-DDDDDDD-COOH | Compound 136 | −8 | 4133.2 | Conforms | 48.3 |
| | DDDDDDD-PEG₂₄-(N₃-DBCO)-Ahx-2B₃W₂ | | | | | |
| 175 | DDDDDDD-PEG₂₄-N₃ | Compound 62 | −8 | 3425.7 | Conforms | 27.8 |
| | DDDDDDD-PEG₂₄-(N₃-DBCO)-Ahx-WWWWW | | | | | |
| 176 | Compound 165 | Compound 136 | −1 | 3901.1 | Conforms | 12.5 |
| | CD22a-PEG₂₄-(N3-DBCO)-Ahx-2B₃W₂ | | | | | |
| 177 | Compound 165 | Compound 62 | −1 | 3193.4 | Conforms | 47.1 |

CD22a-PEG₂₄-(N3-DBCO)-Ahx-WWWWW

Single letter abbreviations are used for amino acid sequences in the above table; X=azidolysine; O=Ornithine; and k=d-lysine. Peptide-based starting materials were manufactured by solid-phase peptide synthesis by Genscript (Piscataway, NJ) and NH₂-PEG₍₅₀₀₀₎-N₃ was obtained from Polysciences (Warrington, PA). Peptide-based starting material sequences are written from N- to C-terminus, and C-terminal —NH₂ and —COOH indicate that the peptides are terminated with either amide or carboxylic acid groups, respectively. Unless otherwise specified, any C-terminal X, azidolysine, is terminated with an amide and is implicit in the sequences (i.e., not shown). Net charge is the predicted net charge of the amphiphile in aqueous buffer at pH 7.4.

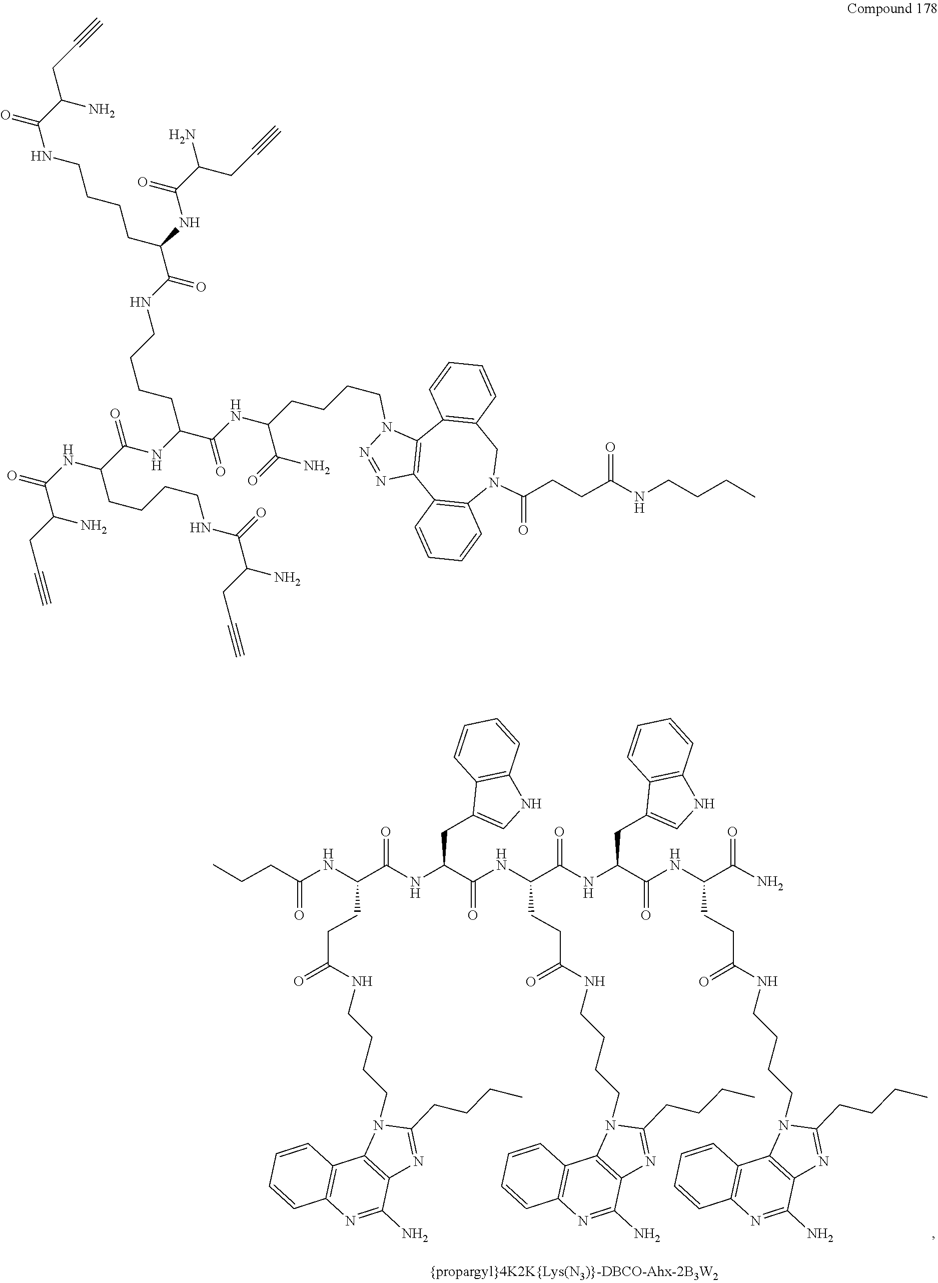

Compound 178, referred to as {propargyl}4K2K{Lys(N3)}-DBCO-Ahx-2B₃W₂ or 2323-Ahx-2B₃W₂ was synthesized by reacting 32.5 mg of Compound 136 (0.02 mmol, 1.05 eq) dissolved in anhydrous DMSO and 16.8 mg of {propargyl}4K2K{Lys(N3)}, which was prepared by solid phase peptide synthesis, (0.02 mmol, 1 eq) as a 100 mg/mL solution in anhydrous DMSO was added. The reaction mixture was stirred overnight at room temperature. HPLC indicated the reaction was complete. Compound 178 was purified on a preparatory HPLC system using a gradient of 25-45% acetonitrile/H₂O (0.05% TFA) over 12 minutes on an Agilent Prep-C18 column, 30×100 mm, 5 μm. The product eluted at ˜3.7 minutes and the resulting fractions were collected, frozen and then lyophilized to obtain 28.1 mg (62.4% yield) of a spectroscopically pure (>99% AUC at 220 nm) white powder. MS (ESI) calculated for C₁₆₀H-206N40019 m/z 2991.6, found 998.6 (M/3+H)⁺.

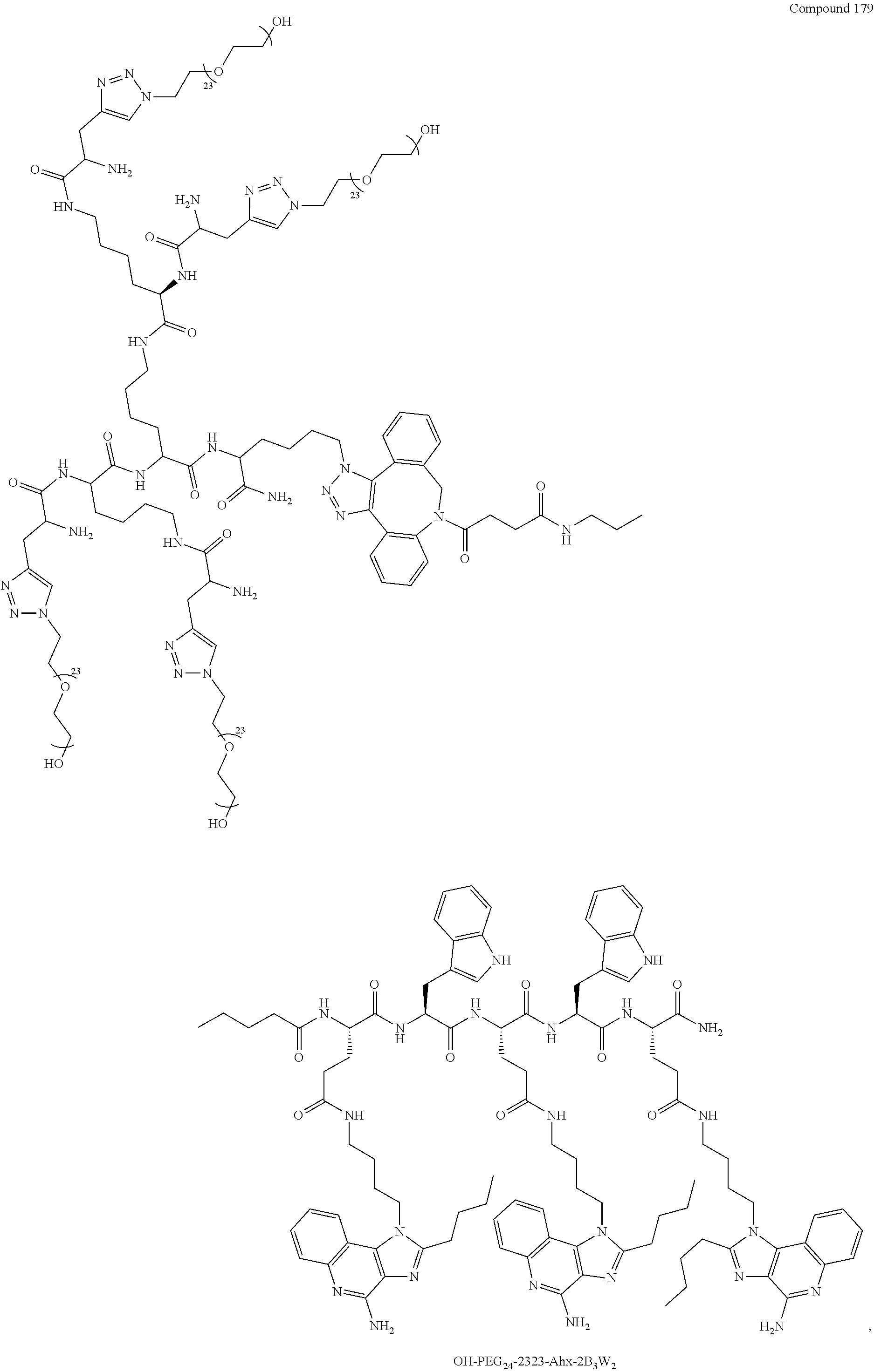

Compound 179, referred to as {OH-PEG₂₄}4-{propargyl}4K2K{Lys(N3)}-DBCO-Ahx-2B₃W₂ or OH-PEG₂₄-2323-Ahx-2B₃W₂, was synthesized by reacting 7.0 mg of Compound 178 (0.0023 mmol, 1 eq) with 18.0 mg of alpha-azido-omega-hydroxy 24(ethylene glycol) or OH-PEG24-N₃ (0.0164 mmol, 7 eq) in 150.6 uL H₂O and 320.1 uL DMSO. To the reaction mixture, 3.24 mg sodium ascorbate (0.00164 mmol, 7 eq) was added. 4.1 mg copper sulfate pentahydrate (0.0163 mmol, 7 eq) and 7.1 mg tris-hydroxypropyltriazolylmethylamine (THPTA) (0.0164 mmol, 7 eq) were combined in a separate vial, and then added to the reaction mixture. The reaction mixture was stirred overnight at room temperature. LC-MS indicated that the reaction was complete. The reaction mixture was diluted in 3 mL H₂O, frozen and lyophilized, and dissolved 600 uL 0.5% Sodium diethyldithiocarbamate/0.5% DIPEA in DMF and was purified by on a preparatory HPLC system using a gradient of 27-47-57% acetonitrile/H₂O (0.05% TFA) over 14 minutes on an Agilent Prep-C18 column, 9.4×100 mm, 5 μm. The product eluted at ˜5 minutes and the resulting fractions were collected, frozen and then lyophilized to obtain 6.6 mg (38.2% yield) of a spectroscopically pure (>99% AUC at 220 nm) off-white solid. MS (ESI) calculated for C₃₅₂H₅₉₄N₅₂O₁₁₅ m/z 7394.9, found 1233.0 (M/6+H)⁺.

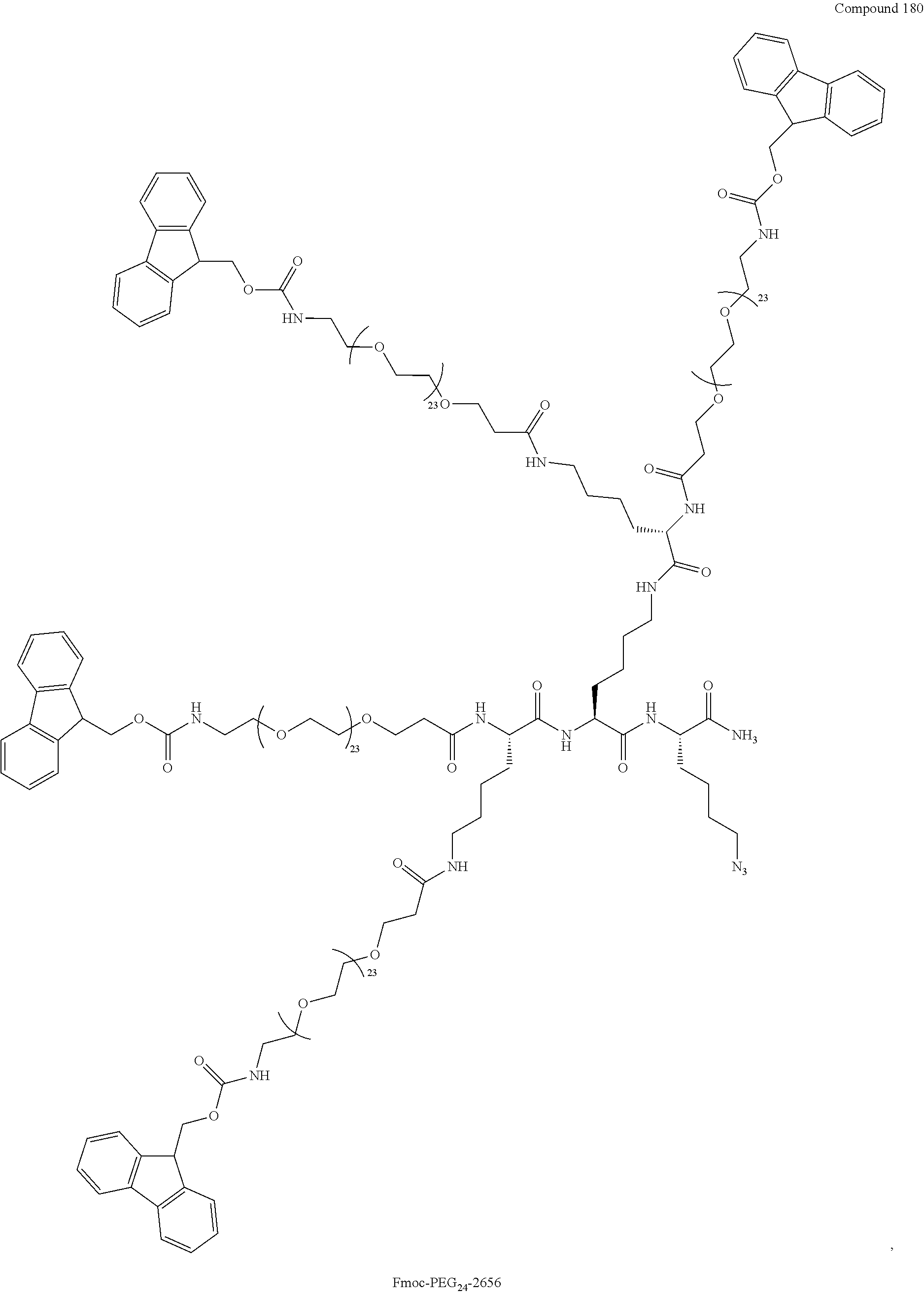

Compound 180, referred to as {Fmoc-PEG₂₄}4-{NH₂}4K2K{Lys(N3)} or Fmoc-PEG₂₄-2656, was synthesized by reacting 73.8 mg Fmoc-PEG₂₄-NHS ester (0.05 mmol, 3.5 eq) with 8 mg of {NH₂}4K2K{Lys(N₃)}, which was prepared by solid phase peptide synthesis, (0.01 mmol, 1 eq). 24.1 uL triethylamine (TEA) (0.17 mmol, 12 eq) was diluted in 6 mL THF and added to the reaction mixture and stirred at room temperature for 24 hours. HPLC indicated that the reaction was 84% complete. MS (ESI) calculated for C₂₈₈H₄₉₃N₁₅O₁₁₂ m/z 5954.3, found 993.8 (M/6+H)⁺.

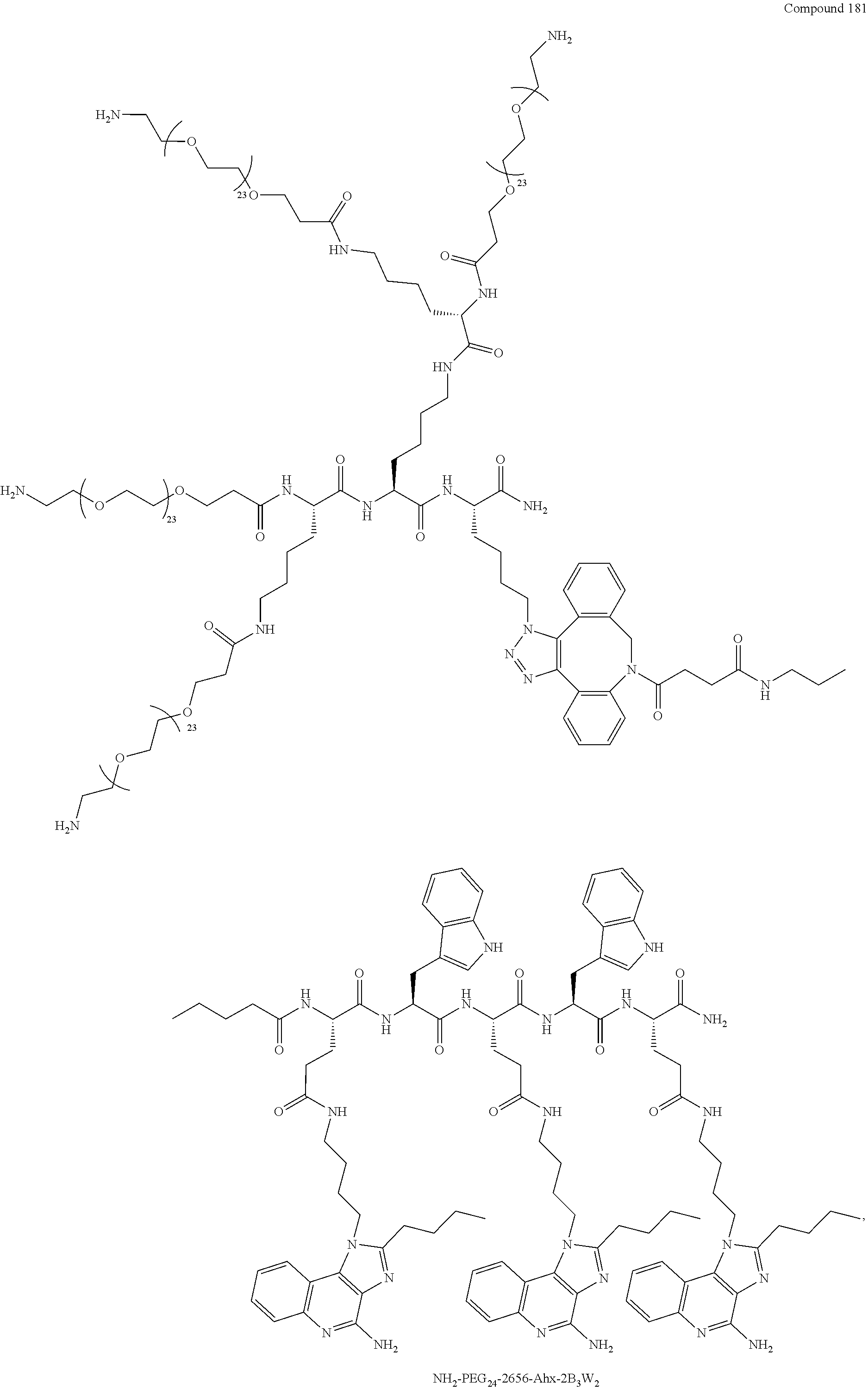

Compound 181, referred to as {NH₂—PEG₂₄}4-{NH₂}4K2K{Lys(N2B₃W₂)}-DBCO-Ahx-2B₃W₂ or NH₂—PEG₂₄-2656-Ahx-2B₃W₂ was synthesized by reacting 7.4 mg of the Fmoc precursor, Compound 180 (0.001 mmol, 1.0 eq) dissolved in anhydrous DMSO and 2.4 mg of Compound 136 (0.001 mmol, 1.05 eq) as a 100 mg/mL solution in anhydrous DMSO was added. The reaction mixture was stirred overnight at room temperature. HPLC indicated the reaction was complete. To the Fmoc protected product was then added piperidine (20% v/v) which was then stirred for 3 hours at room temperature to yield the deprotected product that was then washed with ether. Compound 181 was purified on a preparatory HPLC system using a gradient of 30-40% acetonitrile/H₂O (0.05% TFA) over 12 minutes on an Agilent Prep-C18 column, 30×100 mm, 5 m. The product eluted at ˜5.5 minutes and the resulting fractions were collected, frozen and then lyophilized to obtain a spectroscopically pure (>93.7% AUC at 220 nm) white solid. MS (ESI) calculated for C₃₄₄H₅₉₀N₄₀O₁₁₅ m/z 7122.2, found 1188.6 (M/6+H)⁺.

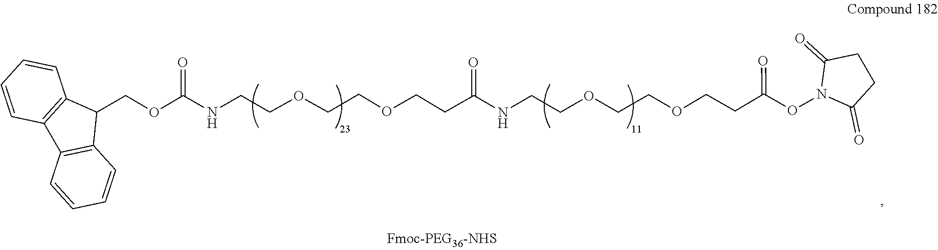

Compound, Fmoc-Peg₃₆-NHS ester was synthesized by combing 30 mg of NH2-Peg12-COOH (0.049 mmol, 1 eq) and 74.7 mg Fmoc-Peg₂₄-NHS ester (0.051 mmol, 1.05 eq) in a vial. 13.5 uL of TEA was added to 1 mL of DMF and then this solution was transferred to the vial containing the Pegs. After 1 h LCMS showed the reaction to be complete. An additional 27.1 uL of TEA was added to the vial followed by 7.27 mg of NHS (0.063 mmol, 1.3 eq). The solution was cooled to 4C with an ice bath and 20.3 mg of HATU (0.053 mmol, 1.1 eq) was added. After 1 h LCMS showed the reaction to be complete. Compound 182 was purified on a preparatory HPLC system using a gradient of 35-60% acetonitrile/H₂O (0.05% TFA) over 12 minutes on an Agilent Prep-C18 column, 30×100 mm, 5 μm. The product eluted at ˜5.5 minutes and the resulting fractions were collected, frozen and then lyophilized to obtain a spectroscopically pure (>93.7% AUC at 220 nm) white solid. MS (ESI) calculated for C₉₇H₁₆₉N₃O₄₃ m/z 2065.4, found 1033.2(M/2+H)⁺.

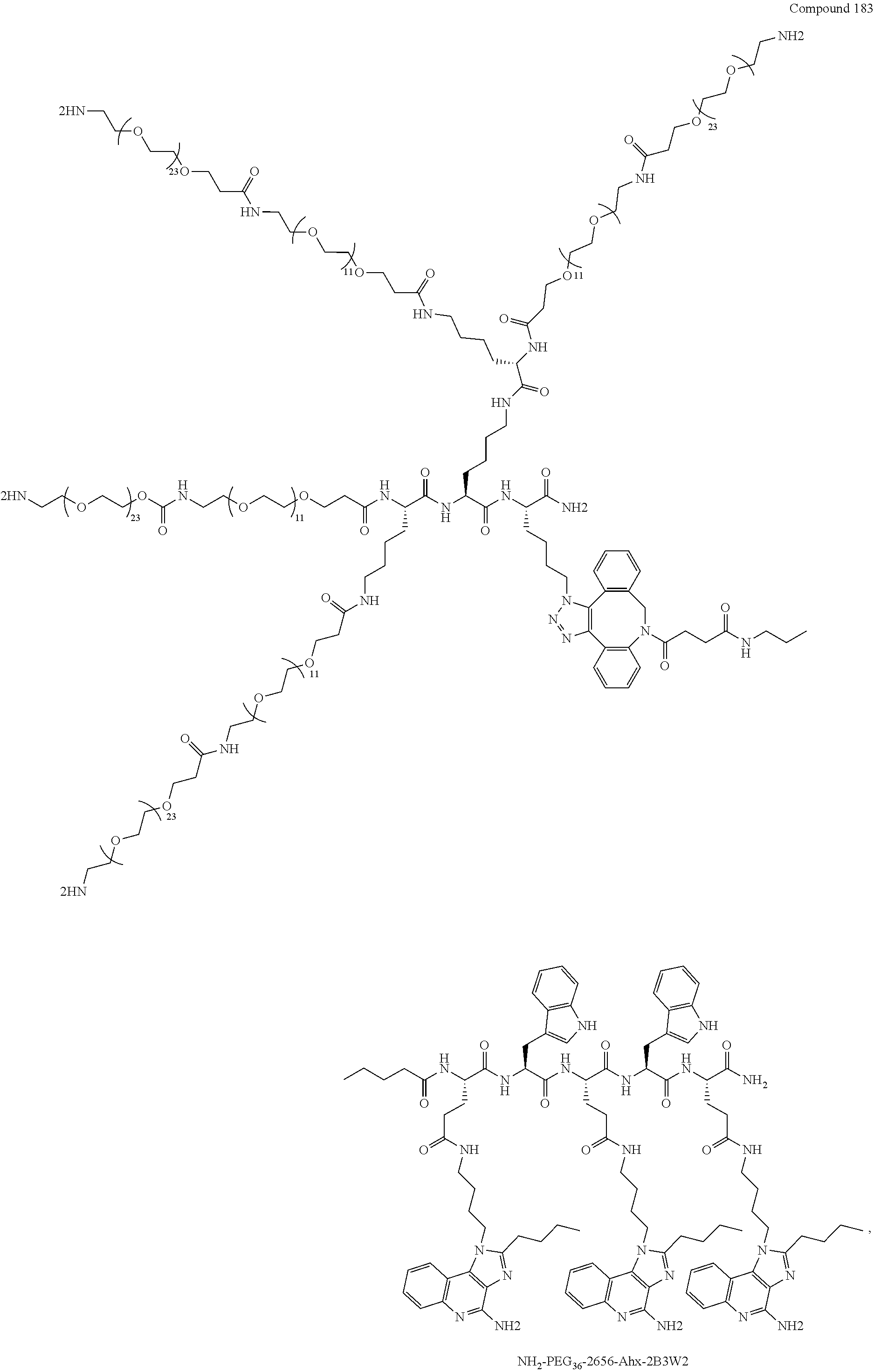

Compound 183, referred to as {NH₂—PEG₃₆}4-{NH₂}4K2K{Lys(N3)}-DBCO-Ahx-2B3W₂ or NH₂—PEG₃₆-2656-Ahx-2B3W₂ was synthesized by reacting 33.4 mg of Compound 182 (0.024 mmol, 4.5 eq) with 3 mg of {NH₂}4K2K{Lys(N₃)}, which was prepared by solid phase peptide synthesis, (0.005 mmol, 1 eq). Triethylamine (TEA) (9.0 uL, 0.06 mmol, 12 eq) was diluted in 6 mL THF and added to the reaction mixture and stirred at room temperature for 3 hours. HPLC indicated that the reaction was 90% complete. The solvent was removed and the fmoc precursor was resuspended in anhydrous DMSO. The fmoc precursor (10.8 mg, 0.001 mmol, 1 eq) was then reacted with 2.2 mg of Compound 136 (0.001 mmol, 1.05 eq) as a 100 mg/mL solution in anhydrous DMSO. The reaction mixture was stirred overnight at room temperature. HPLC indicated the reaction was complete. To the Fmoc protected product was then added piperidine (20% v/v) which was then stirred for 3 hours at room temperature to yield the deprotected product that was then washed with ether. Compound 183 was purified by on a preparatory HPLC system using a gradient of 30-40% acetonitrile/H₂O (0.05% TFA) over 12 minutes on an Agilent Prep-C18 column, 30×100 mm, 5 μm. The product eluted at ˜6 minutes and the resulting fractions were collected, frozen and then lyophilized to obtain a spectroscopically pure (>89.7% AUC at 220 nm) white solid. MS (ESI) calculated for C₄₅₂H₈₀₂N₄₄O₁₆₇ m/z 9519.6, found 865.4 (M/11+H)⁺.

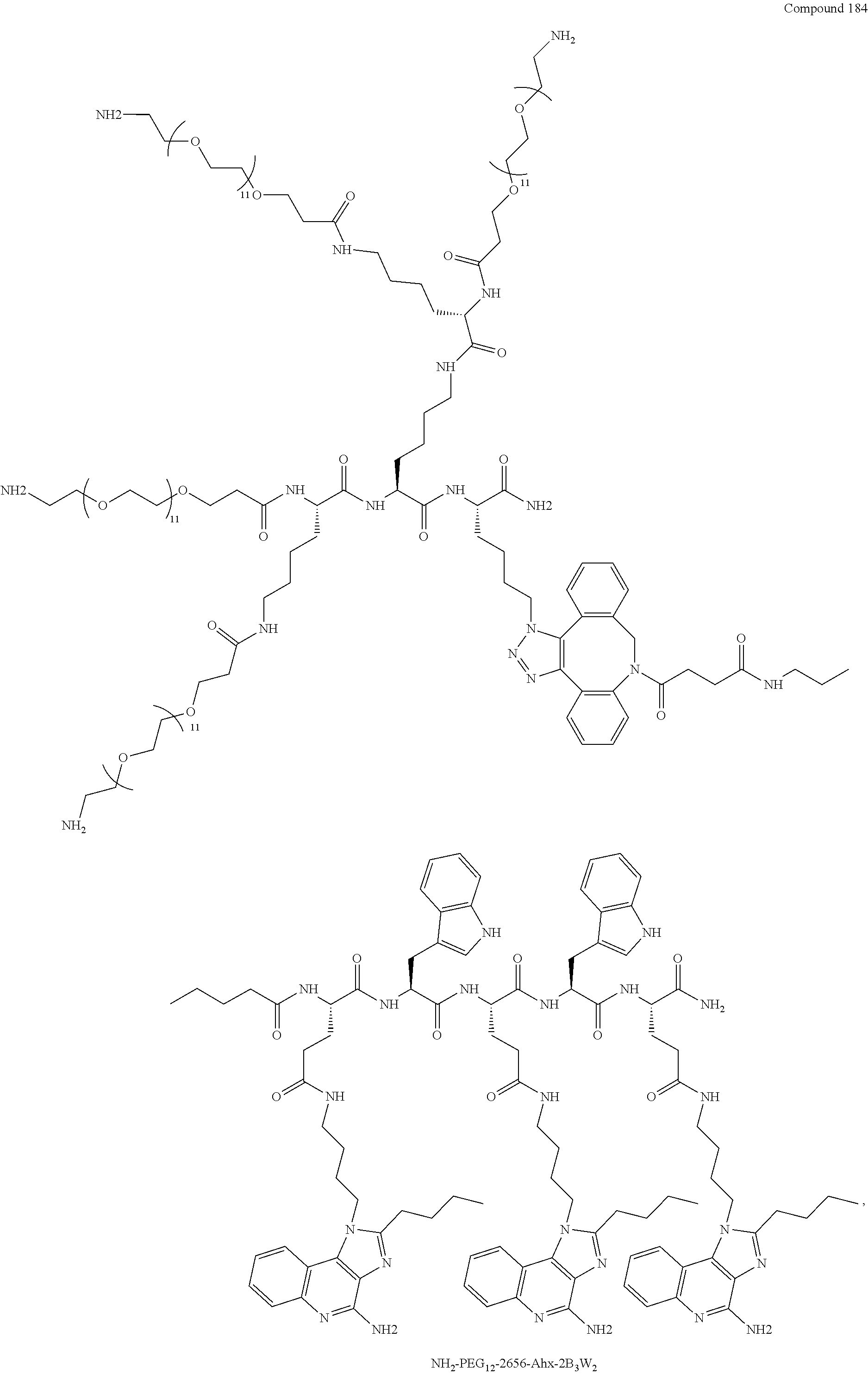

Compound 184, referred to as {NH₂—PEG₁₂}4-{NH₂}4K2K{Lys(N₃)}-DBCO-Ahx-2B₃W₂ or NH₂—PEG₁₂-2656-Ahx-2B₃W₂ was synthesized was synthesized by reacting 15.2 mg Fmoc-PEG₁₂-NHS ester (0.016 mmol, 3.5 eq) with 2.6 mg of {NH₂}4K2K{Lys(N₃)}, which was prepared by solid phase peptide synthesis, (0.005 mmol, 1 eq). Triethylamine (TEA) (20 uL, 0.12 mmol, 30 eq) was diluted in 2 mL THF and added to the reaction mixture and stirred at room temperature for 3 hours. HPLC indicated that the reaction was 90% complete. The solvent was removed and the fmoc precursor was resuspended in anhydrous DMSO. The fmoc precursor (18.6 mg, 0.005 mmol, 1 eq) was then reacted 8 mg of Compound 136 (0.001 mmol, 1.05 eq) as a 100 mg/mL solution in anhydrous DMSO. The reaction mixture was stirred overnight at room temperature. HPLC indicated the reaction was complete. To the Fmoc protected product was then added piperidine (20% v/v) which was then stirred for 3 hours at room temperature to yield the deprotected product that was then washed with ether. Compound 184 was purified by on a preparatory HPLC system using a gradient of 28-38% acetonitrile/H₂O (0.05% TFA) over 12 minutes on an Agilent Prep-C18 column, 30×100 mm, 5 μm. The product eluted at ˜5 minutes and the resulting fractions were collected, frozen and then lyophilized to obtain a spectroscopically pure (95.2% AUC at 220 nm) white solid. MS (ESI) calculated for C₂₄₈H₂₉₈N₄₀O₆₇ m/z 5008.9, found 1253.9 (M/4+H)⁺.

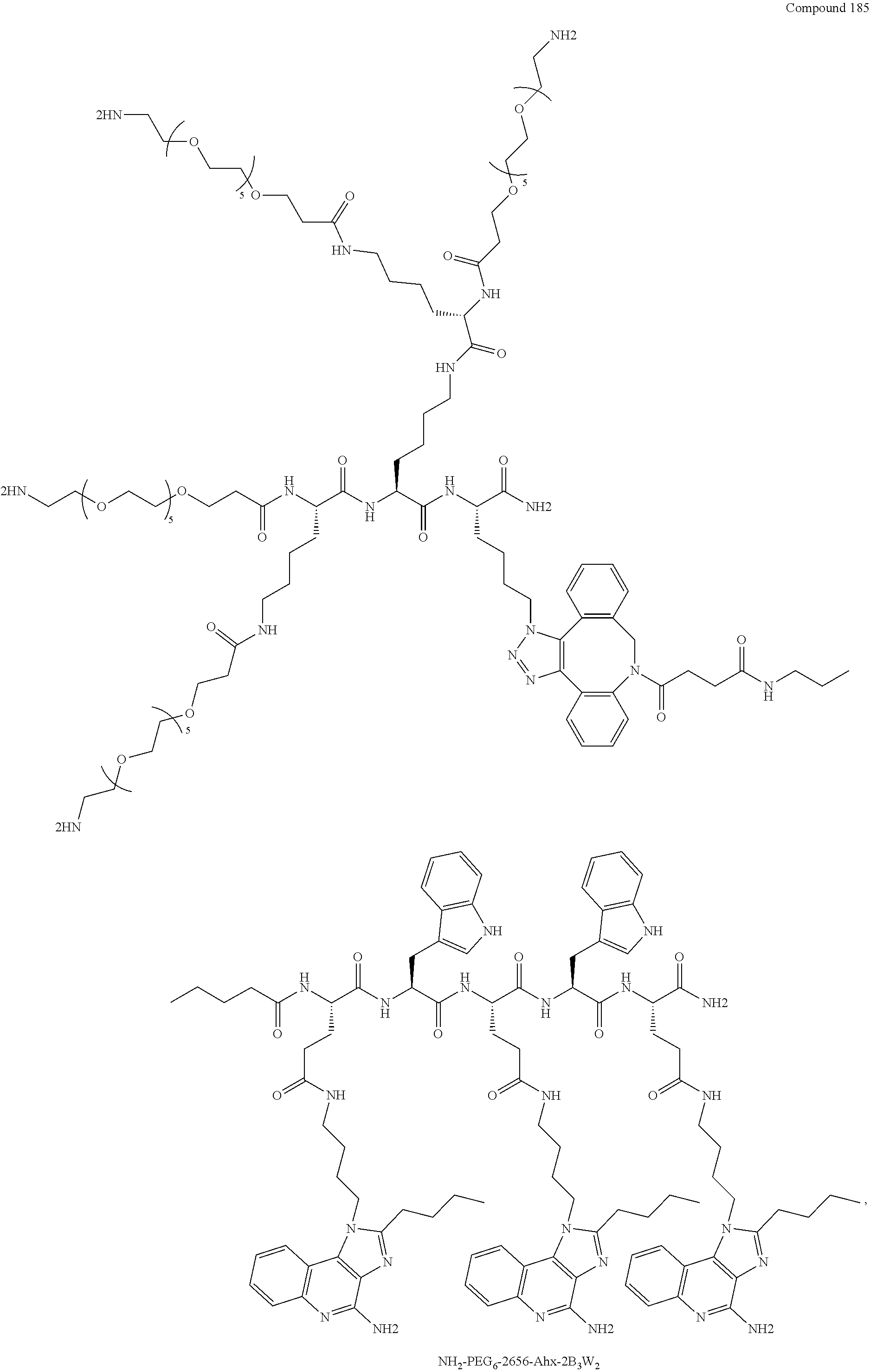

Compound 185, referred to as {NH₂—PEG₆}4-{NH₂}4K2K{Lys(N₃)}-DBCO-Ahx-2B₃W₂ or NH₂—PEG₆-2656-Ahx-2B₃W₂ was synthesized following the same procedure as Compound 184, except Fmoc-PEG₆-NHS was used in place of Fmoc-PEG₁₂-NHS. Compound 185 was purified by on a preparatory HPLC system using a gradient of 18-48% acetonitrile/H₂O (0.05% TFA) over 12 minutes on an Agilent Prep-C18 column, 30×100 mm, 5 μm. The product eluted at ˜6 minutes and the resulting fractions were collected, frozen and then lyophilized to obtain a spectroscopically pure (93.2% AUC at 220 nm) white solid. MS (ESI) calculated for C₂₀₀H₃₀₂N₄₀O₄₃ m/z 3952.3, found 189.7 (M/4+H)⁺.

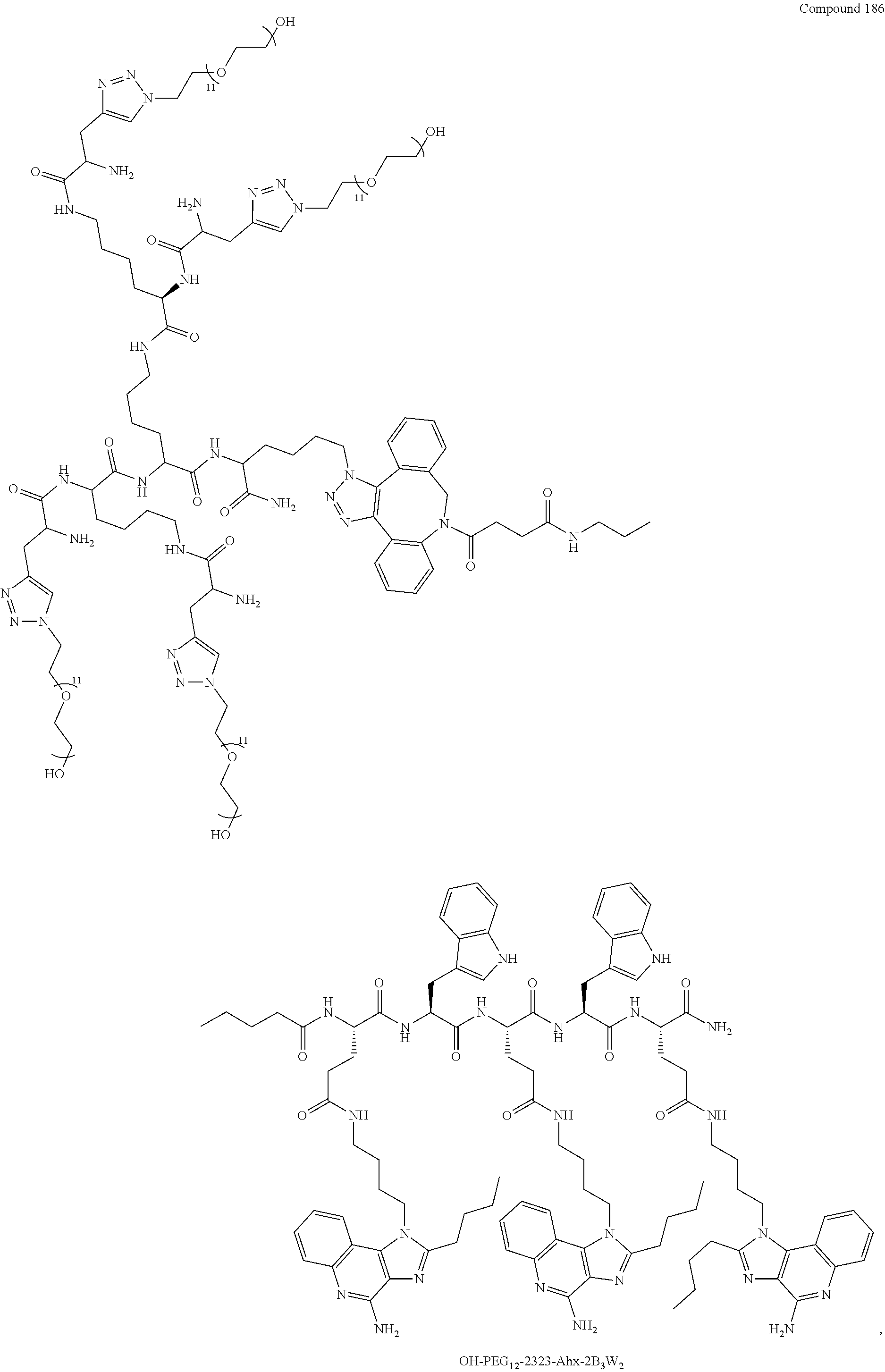

Compound 186, referred to as (OH-PEG₁₂14-{propargyl}4K2K{Lys(N3)}-DBCO-Ahx-2B₃W₂ or OH-PEG₁₂-2323-Ahx-2B₃W₂, was synthesized using the same procedure as Compound 179, except OH-PEG₁₂-N₃ was used in place of OH-PEG₂₄-N₃. Compound 186 was purified by on a preparatory HPLC system using a gradient of 30-50% acetonitrile/H₂O (0.05% TFA) over 14 minutes on an Agilent Prep-C18 column, 9.4×100 mm, 5 μm. The product eluted at ˜4.0 minutes and the resulting fractions were collected, frozen and then lyophilized to obtain 30.2% yield of a spectroscopically pure (91.9% AUC at 220 nm) off-white solid. MS (ESI) calculated for C₂₅₆H₄₀₂N₅₂O₆₇ m/z 5277.0, found 1320.7 (M/4+H)⁺.

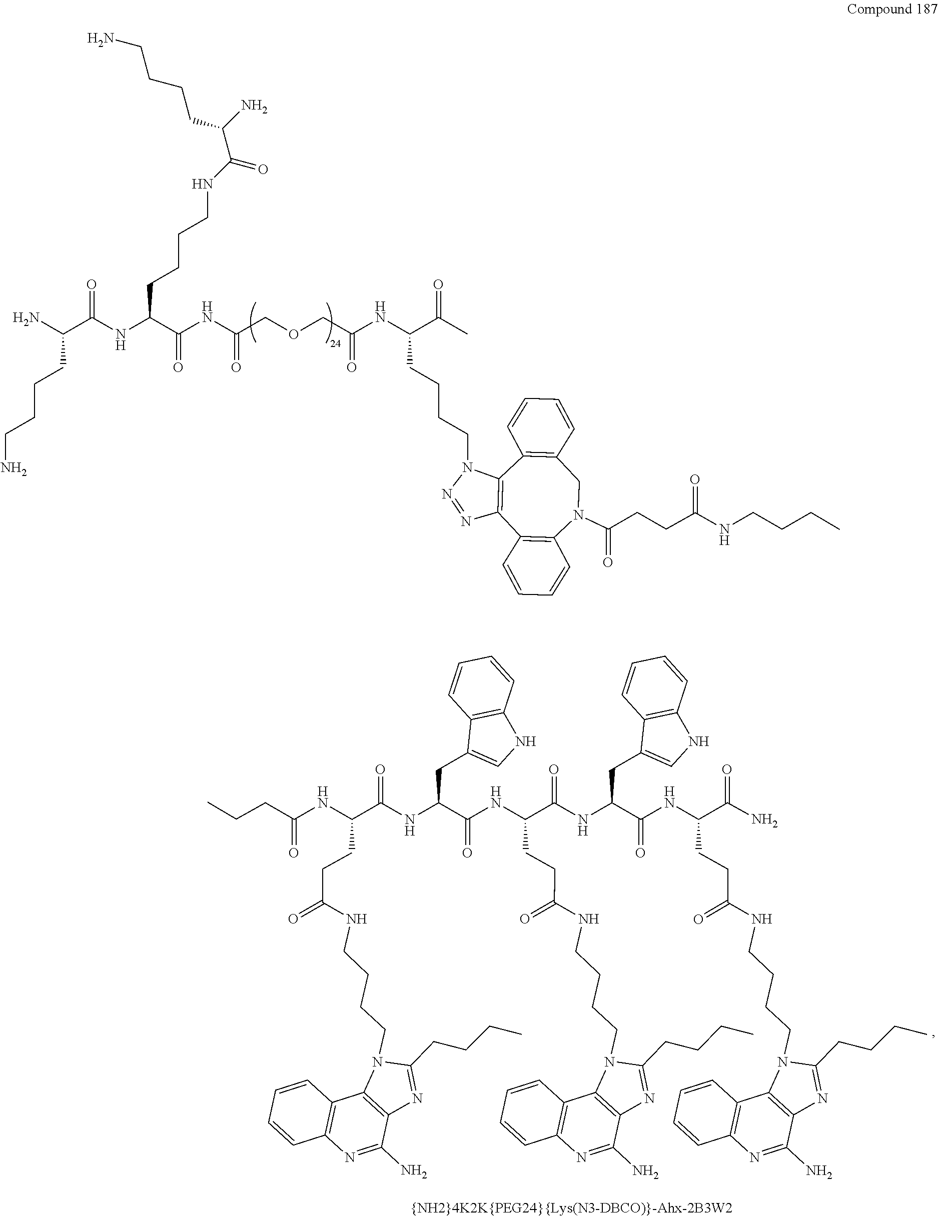

Compound 187, referred to as {NH_{2}4}K2K{PEG24}{Lys(N3-DBCO)}-Ahx-2B3W₂ was synthesized following the same procedure as Compound 181, except {NH₂}4K2K{PEG24}{Lys(N₃)}, which was prepared by solid phase peptide synthesis, was used in place of Compound 180 and resulted in a colorless solution. MS (ESI) calculated for C₁₉₀H₂₈₃N₃₇O₄₁ m/z 3739.1, found 1306.4 (M/2+H)⁺.

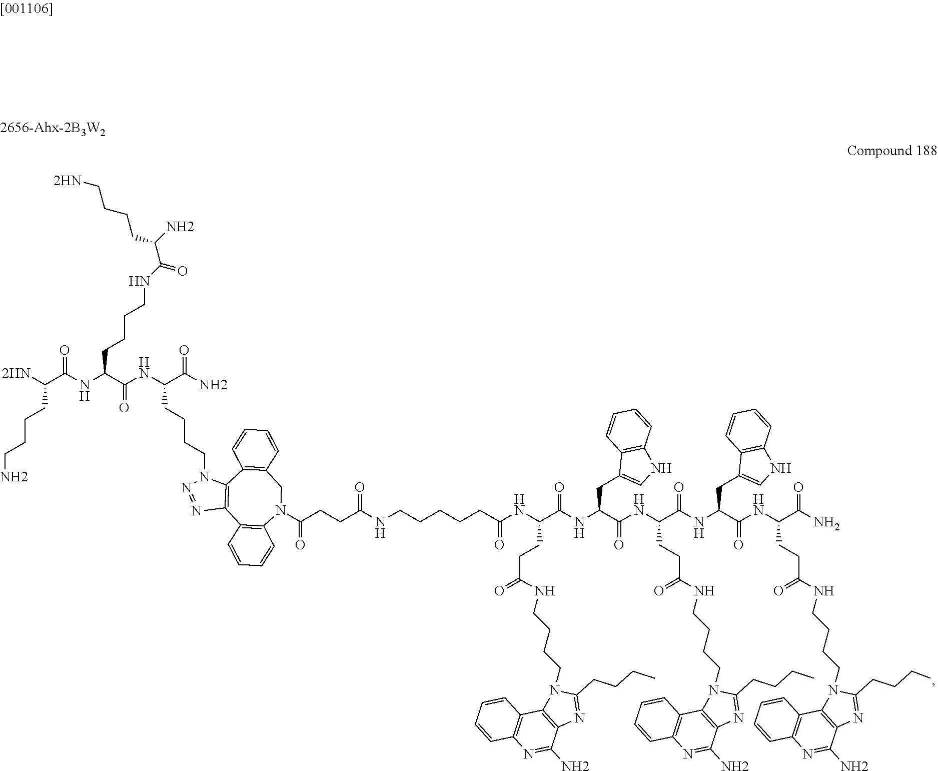

Compound 188, referred to as {NH₂}4K2K{Lys(N₃)}-DBCO-Ahx-2B₃W₂ or 2656-Ahx-2B₃W₂ was synthesized following the same procedure as Compound 181, except {NH₂}4K2K{Lys(N₃)}, which was prepared by solid phase peptide synthesis, was used in place of Compound 180 and resulted in a spectroscopically pure (>98% AUC at 220 nm) colorless solution. MS (ESI) calculated for C₁₄₀H₁₈₆N₃₆O₁₅ m/z 2611.5, found 1306.4 (M/2+H)⁺.

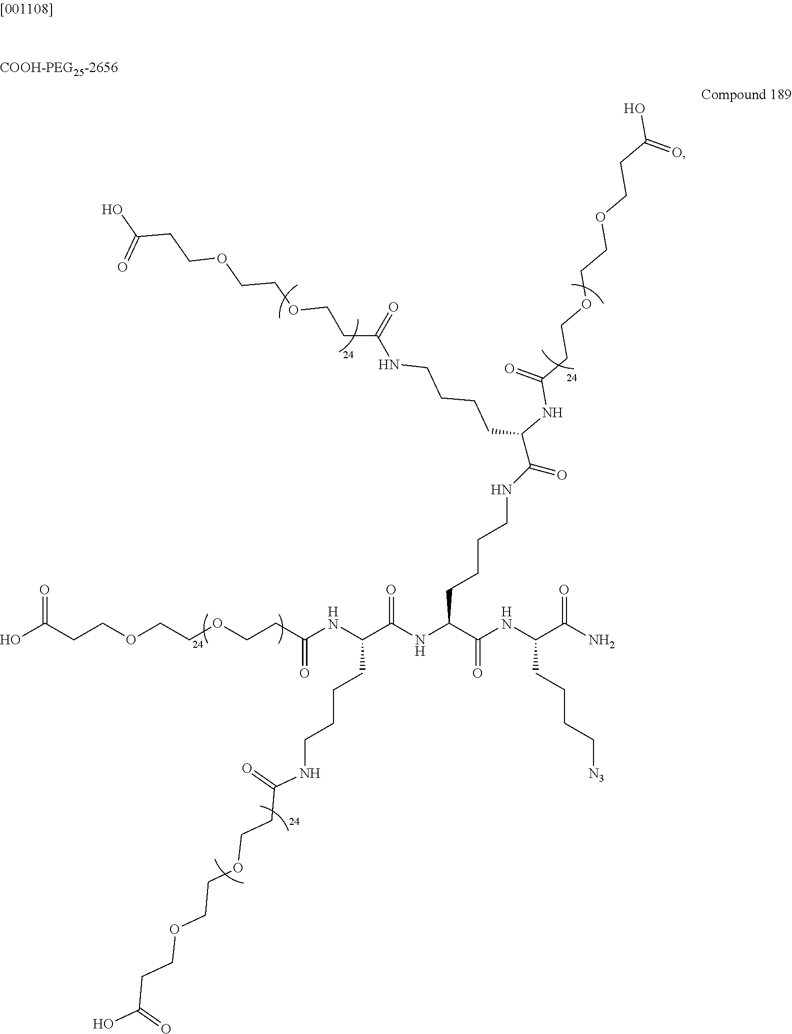

Compound 189, referred to as {COOH-PEG₂₅}4-{NH₂}4K2K{Lys(N₃)} or COOH-PEG₂₅-2656, was synthesized by reacting 59.2 mg COOH-PEG₂₅-NHS ester (0.05 mmol, 2.5 eq) with 10 mg of {NH₂}4K2K{Lys(N3)}, which was prepared by solid phase peptide synthesis, (0.02 mmol, 1 eq). 30.0 uL triethylamine (TEA) (0.21 mmol, 12 eq) was diluted in 400 mL THF and added to the reaction mixture and stirred at room temperature for 24 hours. HPLC indicated that the reaction was complete. Compound 189 was purified by on a preparatory HPLC system using a gradient of 27-47% acetonitrile/H₂O (0.05% TFA) over 12 minutes on an Agilent Prep-C18 column, 30×100 mm, 5 μm. The fractions containing product were collected, frozen and then lyophilized to obtain 57.2 mg (94.9% yield) off-white solid. MS (ESI) calculated for C₂₄₀H₄₆₅N₁₁O₁₁₆ m/z 5358.1, found 1341.0 (M/3+H)⁺.

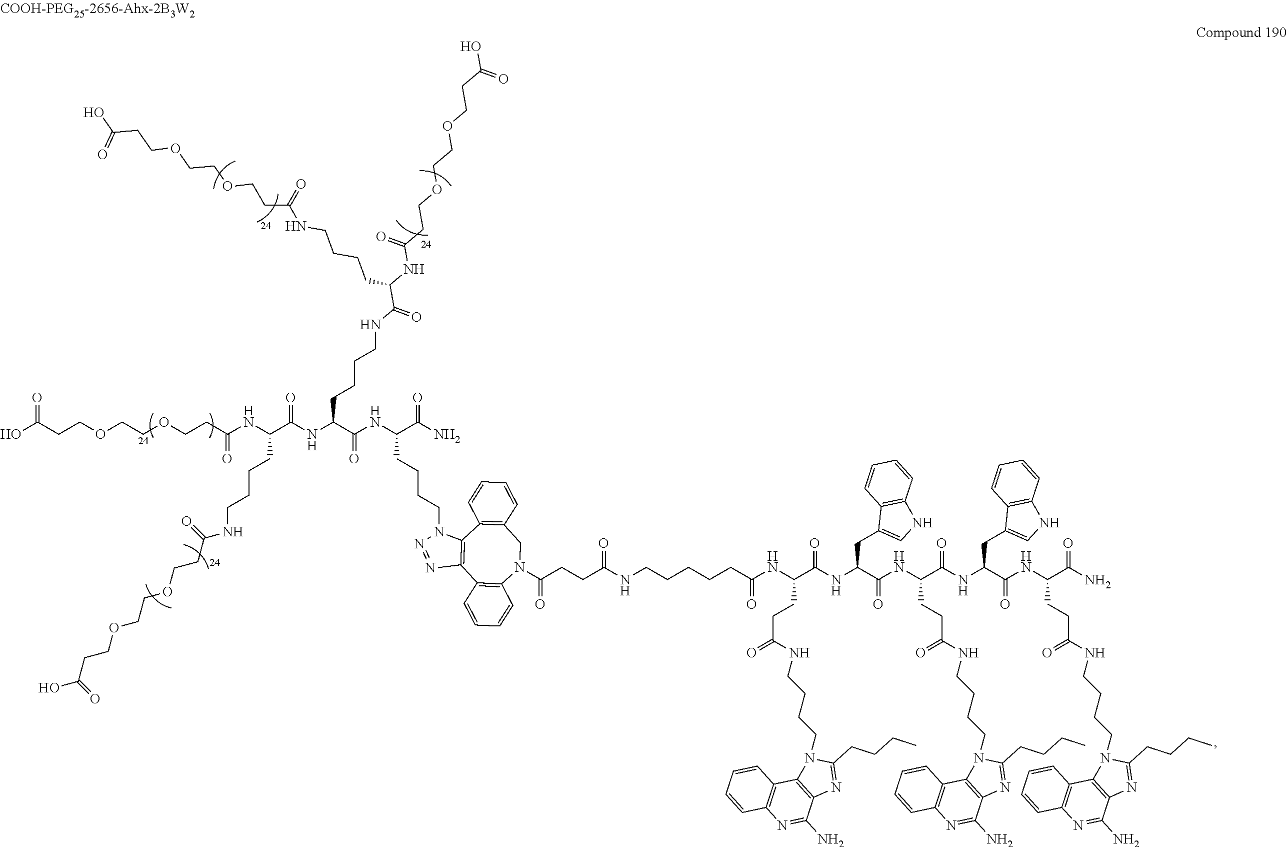

Compound 190, referred to as {COOH-PEG₂₅}4-{NH₂}4K2K{Lys(N₃)}-DBCO-Ahx-2B₃W₂ or COOH-PEG₂₅-2656-Ahx-2B₃W₂ was synthesized by reacting 5.9 mg of Compound 189 (0.001 mmol, 1.0 eq) dissolved in anhydrous DMSO and 2.4 mg of Compound 136 (0.001 mmol, 1.05 eq) as a 100 mg/mL solution in anhydrous DMSO was added. The reaction mixture was stirred overnight at room temperature. HPLC indicated the reaction was complete and resulted in a spectroscopically pure (91.6% AUC at 220 nm) colorless solution. MS (ESI) calculated for C₃₅₆H₆₀₂N₃₆O₁₂₇ m/z 7414.2, found 1484.3 (M/5+H)⁺.

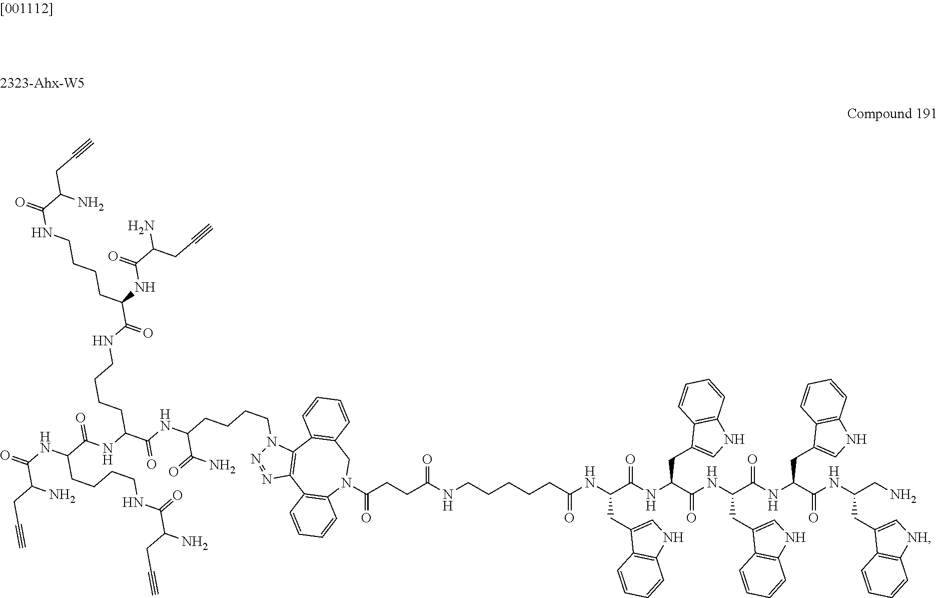 [SMILES: C#CCC(N)C(=O)NCCCCC(NC(=O)C(N)CC#C)C(=O)NC(CCCCNC(=O)[C@@H](CCCCNC(=O)C(N)CC#C)NC(=O)C(N)CC#C)C(=O)NC(CCCCn1nnc2c1-c1ccccc1CN(C(=O)CCC(=O)NCCCCCC(=O)N[C@@H](Cc1c[nH]c3ccccc13)C(=O)N[C@@H](Cc1c[nH]c3ccccc13)C(=O)N[C@@H](Cc1c[nH]c3ccccc13)C(=O)N[C@@H](Cc1c[nH]c3ccccc13)C(=O)N[C@H](CN)Cc1c[nH]c3ccccc13)c1ccccc1-2)C(N)=O]

Compound 191, referred to as {propargyl}4K2K{Lys(N₃)}-DBCO-Ahx-W₅ or 2323-Ahx-W₅ was synthesized by reacting 21.7 mg of Compound 62 (0.02 mmol, 1.05 eq) dissolved in anhydrous DMSO and 28.2 mg of {propargyl}4K2K{Lys(N3)}, which was prepared by solid phase peptide synthesis, (0.02 mmol, 1 eq) as a 100 mg/mL solution in anhydrous DMSO was added. The reaction mixture was stirred overnight at room temperature. HPLC indicated the reaction was complete. Compound 191 was purified on a preparatory HPLC system using a gradient of 25-55% acetonitrile/H₂O (0.05% TFA) over 12 minutes on an Agilent Prep-C18 column, 30×100 mm, 5 μm. The product eluted at ˜5.7 minutes and the resulting fractions were collected, frozen and then lyophilized to obtain 27.2 mg (77.7% yield) white powder. MS (ESI) calculated for C₁₂₄H₁₄₅N₂₇O₁₇ m/z 2284.1, found 1143.3 (M/2+H)⁺.

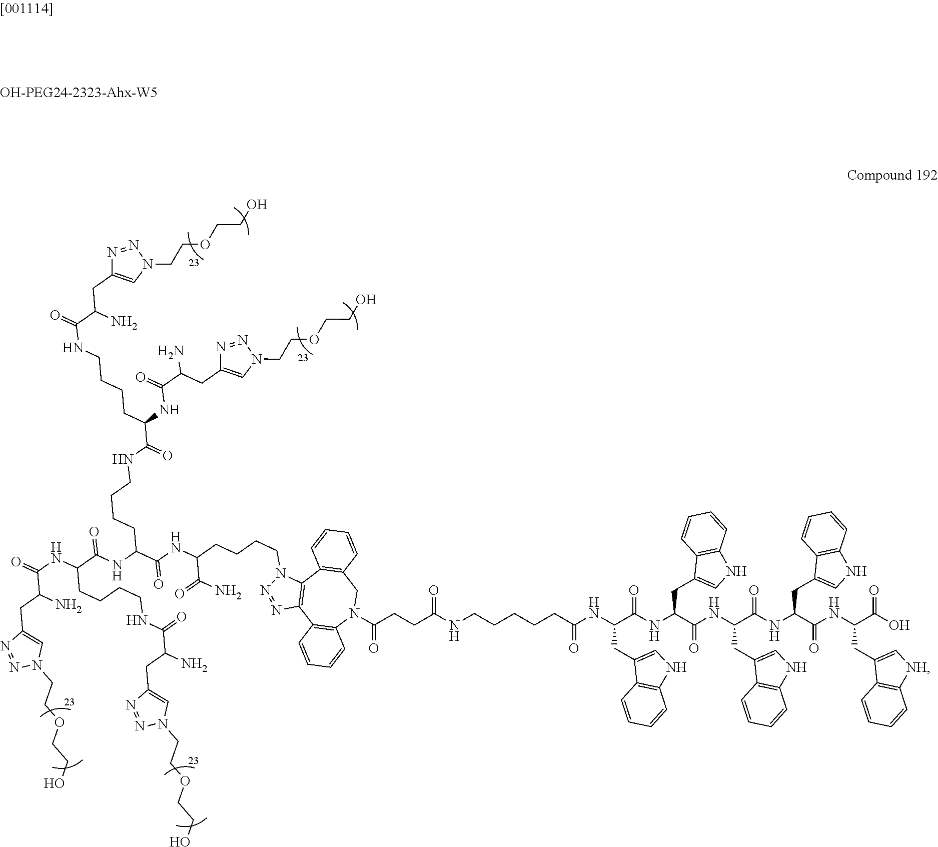

Compound 192, referred to as {OH-PEG₂₄}4-{propargyl}4K2K{Lys(N3)}-DBCO-Ahx-W₅ or OH-PEG24-2323-Ahx-W₅, was synthesized using the same procedure as Compound 179, except Compound 191 was used in place of Compound 178. Compound 192 was purified by on a preparatory HPLC system using a gradient of 31-51-61% acetonitrile/H₂O (0.05% TFA) over 14 minutes on an Agilent Prep-C18 column, 9.4×100 mm, 5 μm. The product eluted at ˜5.5 minutes and the resulting fractions were collected, frozen and then lyophilized to obtain 6.2 mg (35.3% yield) of a spectroscopically pure (98.6% AUC at 220 nm) off-white solid. MS (ESI) calculated for C₃₁₆H₅₃₃N₃₉O₁₁₃ m/z 6682.7, found 1337.8 (M/5+H)⁺.

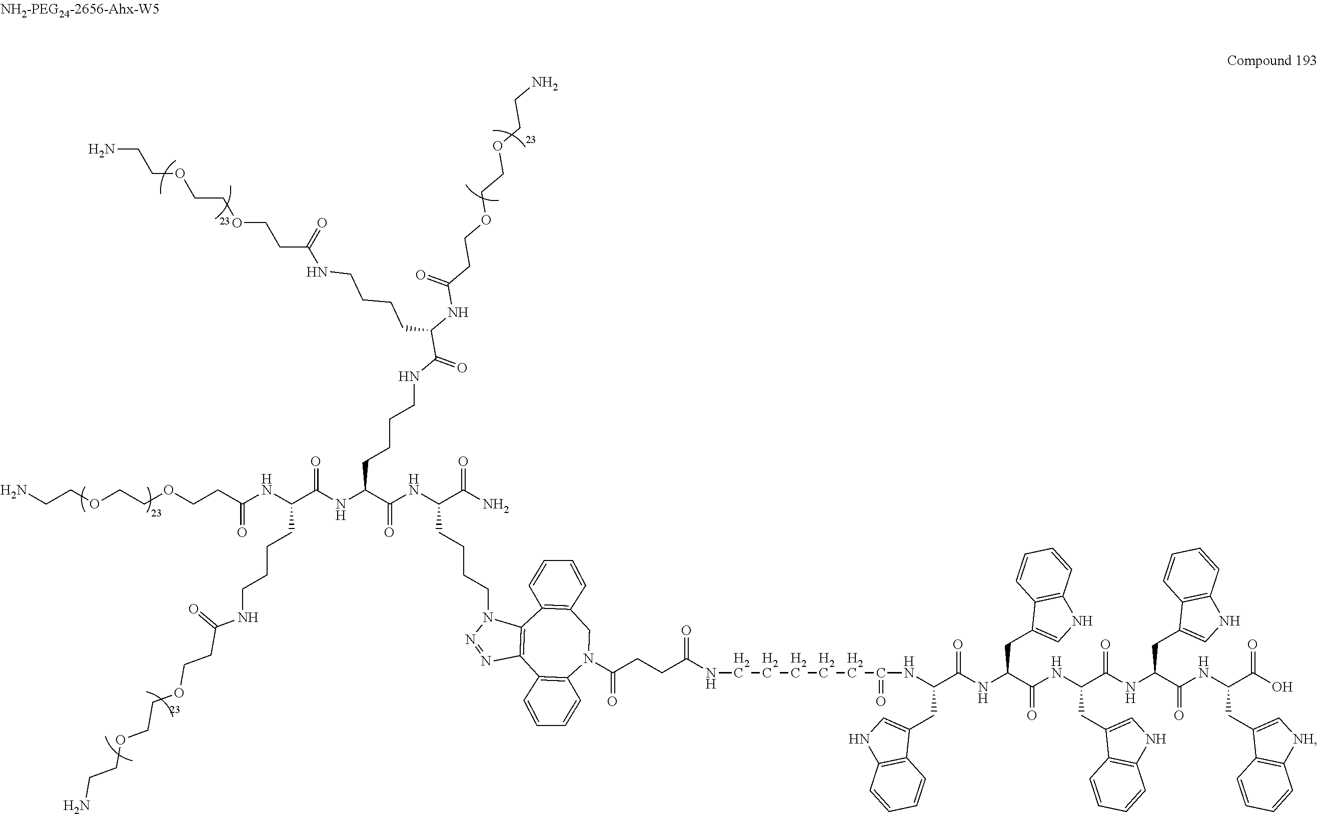

Compound 193, by reacting 12.2 mg of the Fmoc precursor, Compound 180 (0.002 mmol, 1.0 eq) dissolved in anhydrous DMSO and 2.6 mg of Compound 191 (0.002 mmol, 1.05 eq) as a 100 mg/mL solution in anhydrous DMSO was added. The reaction mixture was stirred overnight at room temperature. HPLC indicated the reaction was complete and resulted in a spectroscopically pure (>99% AUC at 254 nm) colorless solution. The Fmoc protected product was then added to 250 μL of a 20% piperidine in DMF solution for 3 hours at room temperature to yield the deprotected product that was then precipitated from 10 mL of ether and centrifuged at 4000 g for 5 minutes. The product was collected as a solid pellet and dissolved in 200 uL DMF, and then crashed once more in ether, followed by drying under vacuum to yield 20.9 mg (>100% yield) of a spectroscopically pure (87.8% AUC at 220 nm) light purple oil was obtained. MS (ESI) calculated for C₃₀₈H₅₂₉N270113 m/z 6414.65, found 1284.2 (M/5+H)⁺.

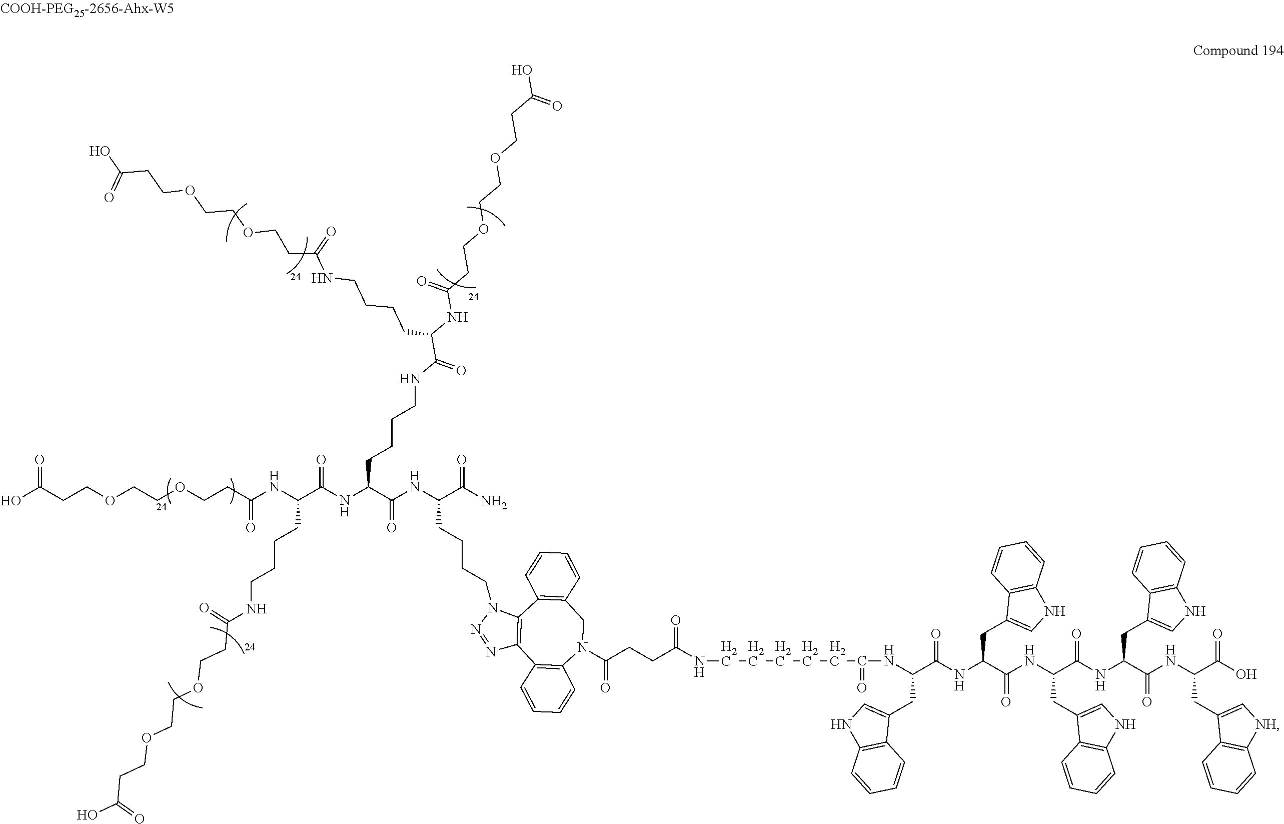

Compound 194, referred to as {COOH-PEG₂₅}4-{NH₂}4K2K{Lys(N3)}-DBCO-Ahx-W₅ or COOH-PEG2s-2656-Ahx-W₅ was synthesized by reacting 4.3 mg of Compound 189 (0.001 mmol, 1.0 eq) dissolved in anhydrous DMSO and 1.6 mg of Compound 62 (0.001 mmol, 1.05 eq) as a 100 mg/mL solution in anhydrous DMSO was added. The reaction mixture was stirred overnight at room temperature. HPLC indicated the reaction was complete and resulted in a spectroscopically pure (94.3% AUC at 220 nm) colorless solution. MS (ESI) calculated for C₃₂₀H₅₄₁N₂₃O₁₂₅ m/z 6706.7, found 1342.6 (M/5+H)⁺.

### Synthesis of Peptide Antigen Conjugates with Brush Architecture

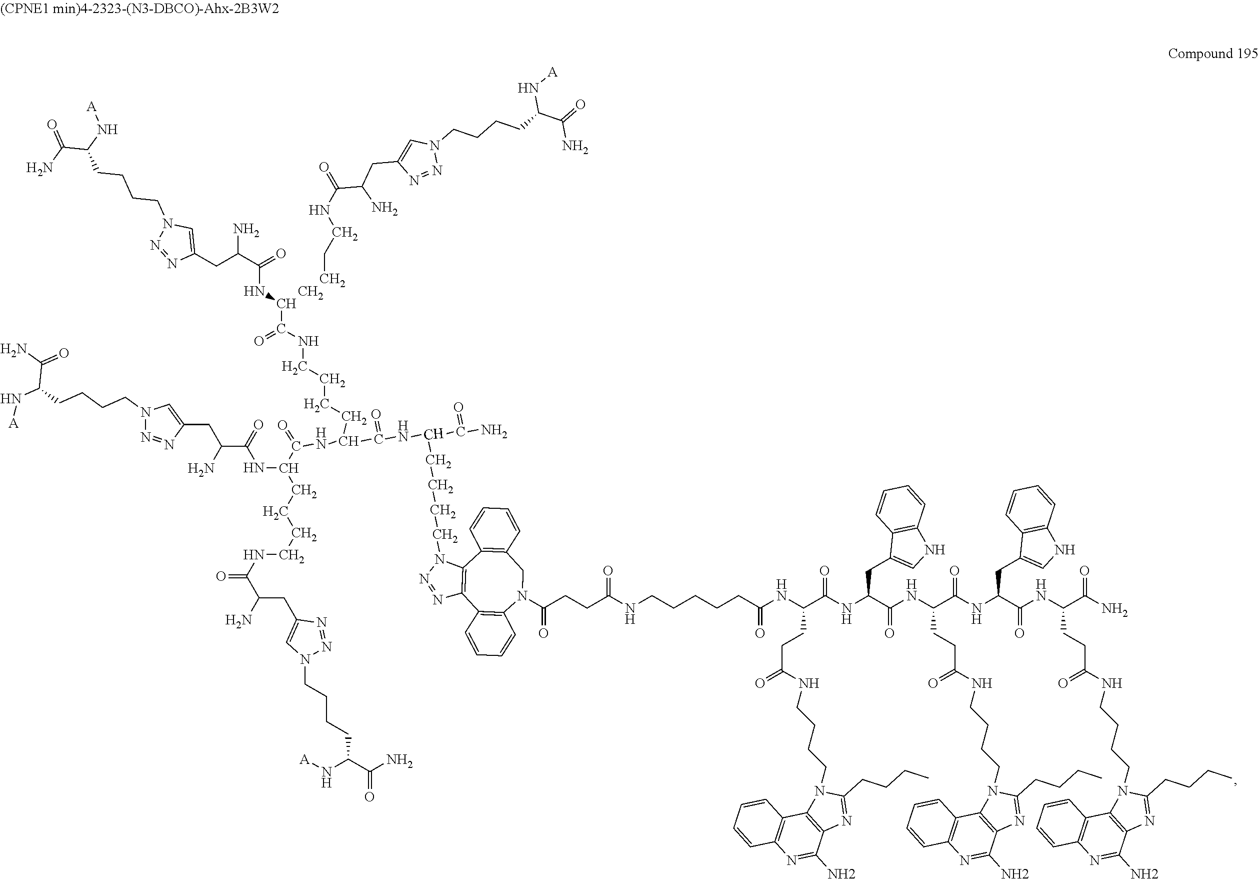

Where A is a peptide antigen sequence SSPYSLHYLX (SEQ ID NO: 104), wherein X is azidolysine.

Compound 195, referred to as (CPNE1 min)4-2323-(N3-DBCO)-Ahx-2B3W₂ or (SSPYSLHYL)4-2323-(N₃-DBCO)-Ahx-2B3W₂, was synthesized by dissolving 0.63 mg of Compound 178 (0.21 umol, 1 eq) with 1.29 mg of Peptide SSPYSLHYLX (SEQ ID NO:104) or CPNE1 min, which was prepared by solid phase peptide synthesis, (1.06 umol, 5 eq) in 15.6 uL H₂O and 320.1 uL DMSO. To the reaction mixture, 0.21 mg sodium ascorbate (1.06 umol, 5 eq) was added. 0.26 mg copper sulfate pentahydrate (0.0163 mmol, 7 eq) and 0.46 mg tris-hydroxypropyltriazolylmethylamine (THPTA) (1.06 umol, 5 eq) were combined in a separate vial, and then added to the reaction mixture. The reaction mixture was stirred overnight at room temperature. LC-MS indicated that the reaction was complete. The reaction mixture was purified by on a preparatory HPLC system using a gradient of 15-50-95% acetonitrile/H₂O (0.05% TFA) over 16 minutes on an Agilent Prep-C18 column, 9.4×100 mm, 5 m. The product eluted between 8 and 11 minutes and the resulting fractions were collected, frozen and then lyophilized to obtain a spectroscopically pure (>90% AUC at 220 nm) off-white solid. MS (ESI) calculated for C₃₈₄H₅₃₄N₁₀₄O₇₉ m/z 7871.15, found 1575 (M/5+H)⁺.

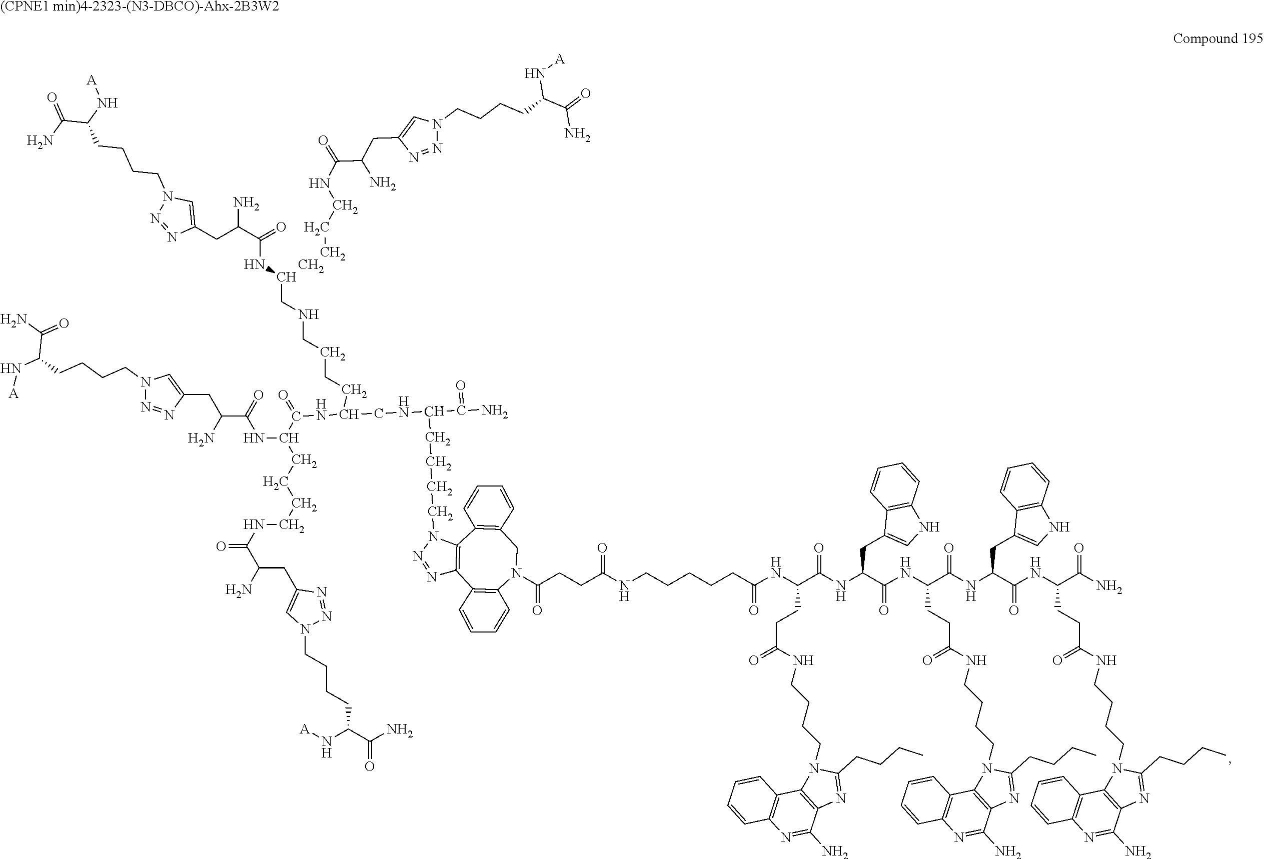

Where A is a peptide antigen sequence DFTGSNGDPSSPYSLHYLSPTGVNEYX (SEQ ID NO:105), wherein X is azidolysine.

Compound 196, referred to as (CPNE1 LP)₄-2323-(N3-DBCO)-Ahx-2B3W₂ was produced using the same method as Compound 195, except Peptide DFTGSNGDPSSPYSLHYLSPTGVNEYX (SEQ ID NO:105) or CPNE1 LP, which was prepared by solid phase peptide synthesis, was used as the starting material. Compound 196 was purified by on a preparatory HPLC system using a gradient of 22-52% acetonitrile/H₂O (0.05% TFA) over 12 minutes on an Agilent Prep-C18 column, 9.4×100 mm, 5 gm. The product eluted between 6.5 and 8 minutes. The resulting fractions were collected, frozen and then lyophilized to obtain a spectroscopically pure (>90% AUC at 220 nm) off-white solid. MS (ESI) calculated for C₆₈₀H₉₄₆N₁₈₀O₁₉₉ m/z 14826.11, found 1479 (M/10+H)⁺.

### Peptide Antigen Conjugates of Formula [S]-[E1]]-A-[E2]-U-H-[D] and H-[D]-U-[E1]]-A-[E2]-[S]

Compounds 197-225 were produced by convergent assembly of a peptide antigen fragment of formula [S]-[E1]-A-[E2]-U1 or U1-[E1]-A-[E2]-[S] with a hydrophobic block fragment of formula U2-H-[D] as previously described in Lynn et al. Nature Biotechnology (2020). In short, the peptide antigen fragment was reacted with 1 molar of equivalent of the hydrophobic block fragment in DMSO at room temperature. The reaction progress was monitored by HPLC and the reaction was stopped at 16 hours after the peptide antigen fragment was completely converted to peptide antigen conjugate.

TABLE 8
Peptide antigen conjugates of formula [S]-[E1]-A-[E2]-U-H-[D] and H-[D]-U-[E1]-
A-[E2]-[S].

| Cmpd | [S]-[E1]-A-[E2]-U1 or U1-[E1]-A-[E2]-[S] | U2-H[D] | Theo. | Found |
|---|---|---|---|---|
| # | Product | | MW | MW |
| 197 | SSPYSLHYLX (SEQ ID NO: 104) | Compound 136 | 3275.45 | |
| | SSPYSLHYL-(N3-DBCO)-Ahx-2B3W2 | | | |
| 198 | DFTGSNGDPSSPYSLHYLSPTGVNEYX (SEQ ID NO: 105) | Compound 136 | 5014.18 | 1254.7 |
| | DFTGSNGDPSSPYSLHYLSPTGVNEY-(N3-DBCO)-Ahx-2B3W2 | | | (m/4) |
| 199 | KARDETAALLNSAVLGAAPLFVPPADX (SEQ ID NO: 402) | Compound 136 | 4817.25 | 1205.6 |
| | KARDETAALLNSAVLGAAPLFVPPAD-(N3-DBCO)-Ahx-2B3W2 | | | (m/4) |
| 200 | AALLNSAVLX (SEQ ID NO: 403) | Compound 136 | 3080.3 | |
| | AALLNSAVL-(N3-DBCO)-Ahx-2B3W2 | | | |
| 201 | ASMTNMELMX (SEQ ID NO: 404) | Compound 136 | 3236.5 | |
| | ASMTNMELM-(N3-DBCO)-Ahx-2B3W2 | | | |
| 202 | GIPVHLELASMTNMELMSSIVHQQVFPTX (SEQ ID NO: 405) | Compound 136 | 5319.93 | 1774 |
| | GIPVHLELASMTNMELMSSIVHQQVFPT-(N3-DBCO)-Ahx-2B3W2 | | | (m/3) |
| 203 | KKKVRGIPVHLELASMTNMELMSSIVHQQVFPTX (SEQ ID | Compound 136 | 5959.76 | 1491 |
| | NO: 406) | | | (m/4) |
| | KKKVRGIPVHLELASMTNMELMSSIVHQQVFPT-(N3-DBCO)-Ahx-2B3W2 | | | |
| 204 | KKKKVRDFTGSNGDPSSPYSLHYLSPTGVNEYX (SEQ ID | Compound 136 | 5782.19 | 1446.6 |
| | NO: 407) | | | (m/4) |
| | KKKKVRDFTGSNGDPSSPYSLHYLSPTGVNEY-(N3-DBCO)-Ahx-2B3W2 | | | |
| 205 | KKKKKVRGIPVHLELASMTNMELMSSIVHQQVFPTX (SEQ | Compound 136 | 6216.11 | 1244.2 |
| | ID NO: 408) | | | (m/5) |
| | KKKKKVRGIPVHLELASMTNMELMSSIVHQQVFPT-(N3-DBCO)-Ahx-2B3W2 | | | |
| 206 | KKKKKKVRDFTGSNGDPSSPYSLHYLSPTGVNEYX (SEQ | Compound 136 | 6039.44 | 1007.6 |
| | ID NO: 409) | | | (m/6) |
| | KKKKKKVRDFTGSNGDPSSPYSLHYLSPTGVNEY-(N3-DBCO)-Ahx-2B3W2 | | | |
| 207 | Azp-PEG12-TESNKKFLPFQQFGRDIA-NH₂ | Compound 136 | 4807.22 | 1203 |
| | 2B3W2-Ahx-(DBCO-N3)-PEG12-TESNKKFLPFQQFGRDIA-NH₂ | | | (m/4) |
| 208 | Azp-PEG12-KKTESNKKFLPFQQFGRDIAKPK-NH₂ | Compound 136 | 5417.02 | 1084.6 |
| | 2B3W2-Ahx-(DBCO-N3)-PEG12-KKTESNKKFLPFQQFGRDIAKPK-NH₂ | | | (m/5) |
| 209 | Azp-KK-PEG12-TESNKKFLPFQQFGRDIAKPK-NH₂ (SEQ ID | Compound 136 | 5516.14 | 1380 |
| | NO: 412) | | | (m/4) |
| | 2B3W2-Ahx-(DBCO-N3)-KK-PEG12-TESNKKFLPFQQFGRDIAKPK-NH₂ | | | |
| 210 | Azp-KK-PEG12-TESNKKFLPFQQFGRDIA-PEG4-KK-NH₂ | Compound 136 | 5666.31 | 1417.6 |
| | 2B3W2-Ahx-(DBCO-N3)-KK-PEG12-TESNKKFLPFQQFGRDIA-PEG4-KK-NH₂ | | | (m/4) |
| 211 | Azp-TESNKKFLPFQQFGRDIA-NH₂ | Compound 136 | 4306.47 | 1077.9 |
| | 2B3W2-Ahx-(DBCO-N3)-TESNKKFLPFQQFGRDIA-NH₂ | | | (m/4) |
| 212 | Azp-PEG12-SQILPDPSKPSKRSFIEDLLFNKVTLADAGF-NH₂ | Compound 136 | 6115.73 | 1020.4 |
| | 2B3W2-Ahx-(DBCO-N3)-PEG12-SQILPDPSKPSKRSFIEDLLFNKVTLADAGF | | | (m/6) |
| 213 | Azp-PEG12- | Compound 136 | 6725.51 | 961.9 |
| | KKSQILPDPSKPSKRSFIEDLLFNKVTLADAGFKPK-NH₂ | | | (m/7) |
| | 2B3W2-Ahx-(DBCO-N3)-PEG12-KKSQILPDPSKPSKRSFIEDLLFNKVTLADAGFKPK | | | |
| 214 | Azp-PEG12-SQILPDPSKPSKRSFIEDLLFNKVT-NH₂ | Compound 136 | 5541.06 | 1386.4 |
| | 2B3W2-Ahx-(DBCO-N3)-PEG12-SQILPDPSKPSKRSFIEDLLFNKVT-NH₂ | | | (m/4) |
| 215 | Azp-PEG12-KKSQILPDPSKPSKRSFIEDLLFNKVTKPK-NH₂ | Compound 136 | 6150.9 | 1026.2 |
| | 2B3W2-Ahx-(DBCO-N3)-PEG12-KKSQILPDPSKPSKRSFIEDLLFNKVTKPK-NH₂ | | | (m/6) |
| 216 | Azp-PEG12-VEGFNCYFPLQSYGFQPTNGVGYQ-NH₂ | Compound 136 | 5396.74 | 1350.3 |
| | 2B3W2-Ahx-(DBCO-N3)-PEG12-VEGFNCYFPLQSYGFQPTNGVGYQ-NH₂ | | | (m/4) |
| 217 | Azp-PEG12-KKVEGFNCYFPLQSYGFQPTNGVGYQKPK-NH₂ | Compound 136 | 6006.59 | 1002.2 |
| | 2B3W2-Ahx-(DBCO-N3)-PEG12-KKVEGFNCYFPLQSYGFQPTNGVGYQKPK-NH₂ | | | (m/6) |
| 218 | MEVGWYRSPFSRVVHLYRNGKSPVZX (SEQ ID NO: 410) | Compound 62 | 4524.23 | 1131.8 |
| | MEVGWYRSPFSRVVHLYRNGKSPVZ-(N3-DBCO)-Ahx-W5 | | | (m/4) |
| 219 | MEVGWYRSPFSRVVHLYRNGKSPVZX (SEQ ID NO: 410) | Compound 136 | 5231.74 | 1047.8 |
| | MEVGWYRSPFSRVVHLYRNGKSPVZ-(N3-DBCO)-Ahx-2B3W2 | | | (m/5) |
| 220 | DFTGSNGDPSSPYSLHYLSPTGVNEYX (SEQ ID NO: 105) | Compound 62 | 4306.67 | 1436.2 |
| | DFTGSNGDPSSPYSLHYLSPTGVNEY-(N3-DBCO)-Ahx-W5 | | | (m/3) |
| 221 | KARDETAALLNSAVLGAAPLFVPPADX (SEQ ID NO: 402) | Compound 62 | 4109.74 | 1370.6 |
| | KARDETAALLNSAVLGAAPLFVPPAD-(N3-DBCO)-Ahx-W5 | | | (m/3) |
| 222 | GIPVHLELASMTNMELMSSIVHQQVFPTX (SEQ ID NO: 405) | Compound 62 | 4612.42 | 1538.4 |
| | GIPVHLELASMTNMELMSSIVHQQVFPT-(N3-DBCO)-Ahx-W5 | | | (m/3) |
| 223 | GRVLELFRAAQLANDVVLQIMELCGATRX (SEQ ID | Compound 62 | 4589.4 | 1529.8 |
| | NO: 411) | | | (m/3) |
| | GRVLELFRAAQLANDVVLQIMELCGATR-(N3-DBCO)-Ahx-W5 | | | |
| 224 | GRVLELFRAAQLANDVVLQIMELCGATRX (SEQ ID | Compound 136 | 5296.81 | 1766.6 |
| | NO: 411) | | | (m/3) |
| | GRVLELFRAAQLANDVVLQIMELCGATR-(N3-DBCO)-Ahx-2B3W2 | | | |
| 225 | AKFVAAWTLKAAASPVZX (SEQ ID NO: 412) | Compound 136 | 3997.29 | 1333.6 |

| | |
|---|---|
| AKFVAAWTLKAAASPVZ-(N3-DBCO)-Ahx-2B3W2 | (m/3) |

Single letter abbreviations are used for amino acid sequences in the above table; X = azidolysine and Z = citrulline. Peptide-based starting materials were manufactured by solid-phase peptide synthesis by Genscript (Piscataway, NJ). Sequences for peptide-based starting materials are written from N- to C- terminus, and C-terminal NH₂ indicates that the peptide is terminated with an Amide. Unless otherwise specified, any C-terminal X, azidolysine, is terminated with an amide and is implicit in the sequences (i.e., not shown). N-terminal Azp = azidopentanoic acid.

### Peptide Antigen Conjugates of Formula A-[E2]-H and H-[E1]-A

Compounds 226-241 in Table 9 were synthesized by solid phase peptide synthesis.

TABLE 9
Peptide antigen conjugates of formula A-[E2]-H and H-[E1]-A produced by SPPS.

| | | Theo- | |
|---|---|---|---|
| Cmpd | Peptide antigen conjugate sequence (H = (F′)n, | retical | Found |
| # | where n = 5 or 10 | Mw | (MS-ESI) |
| 226 | GIPVHLELASMTNMELMSSIVHQQVFPTF′F′F′F′F′F′F′F′F′F′ (SEQ | 4731.38 | Conforms |
| | ID NO: 413) | | |
| 227 | KARDETAALLNSAVLGAAPLFVPPAD F′F′F′F′F′F′F′F′F′F′ (SEQ | 4228.86 | Conforms |
| | ID NO: 414) | | |
| 228 | DFTGSNGDPSSPYSLHYLSPTGVNEY F′F′F′F′F′F′F′F′F′F′ (SEQ ID | 4425.63 | Conforms |
| | NO: 415) | | |
| 229 | ASMTNMELMF′F′F′F′F′F′F′F′F′F′ (SEQ ID NO: 416) | 2647.99 | Conforms |
| 230 | AALLNSAVLF′F′F′F′F′F′F′F′F′F′ (SEQ ID NO: 417) | 2491.78 | Conforms |
| 231 | SSPYSLHYLF′F′F′F′F′F′F′F′F′F′ (SEQ ID NO: 418) | 2686.91 | Conforms |
| 232 | ASMTNMELMSPVZF′F′F′F′F′F′F′F′F′F′ (SEQ ID NO: 419) | 3088.49 | Conforms |
| 233 | AALLNSAVLSPVZF′F′F′F′F′F′F′F′F′F′ (SEQ ID NO: 420) | 2932.28 | Conforms |
| 234 | SSPYSLHYLSPVZF′F′F′F′F′F′F′F′F′F′ (SEQ ID NO: 421) | 3127.41 | Conforms |
| 235 | GIPVHLELASMTNMELMSSIVHQQVFPTF′F′F′F′F′ (SEQ ID | 3920.52 | Conforms |
| | NO: 422) | | |
| 236 | GRVLELFRAAQLANDVVLQIMELCGATRF′F′F′F′F′ (SEQ ID | 3897.59 | Conforms |
| | NO: 423) | | |
| 237 | KARDETAALLNSAVLGAAPLFVPPADF′F′F′F′F′ (SEQ ID | 3417.79 | Conforms |
| | NO: 424) | | |
| 238 | DFTGSNGDPSSPYSLHYLSPTGVNEYF′F′F′F′F′ (SEQ ID | 3614.76 | Conforms |
| | NO: 425) | | |
| 239 | F′F′F′F′F′F′F′F′F′F′GIPVHLELASMTNMELMSSIVHQQVFPTS | 4915.58 | Conforms |
| | P (SEQ ID NO: 426) | | |
| 240 | F′F′F′F′F′SKLLSFMAPIDHTTMSDDARTELFRS (SEQ ID NO: 427) | 3779.36 | Conforms |
| 241 | GRVLELFRAAQLANDVVLQIMELCGATRSPF′F′F′F′F′F′F′F′F′ | 4892.57 | Conforms |
| | F′ (SEQ ID NO: 428) | | |

Single letter abbreviations are used for amino acid sequences in the above table; F′ = para-aminophenylalanine and Z = citrulline. Peptides were produced by solid phase peptide synthesis by Genscript (Piscataway, NJ) and purity and identity were confirmed by HPLC and mass spectrometry; “conforms” indicates that mass spectrum conformed to the expected m/z. Unless otherwise specified all of the sequences are terminated with an amide at the C-terminus.

Additional Amphiphiles of formula S-B-U-H-[D]

Compounds 242-248 (Table 10) were produced in a similar manner as for those described in Table 7.

TABLE 10
amphiphiles of formula S-B-U-H-[D]

| | | Starting U2-H[(D)] | | | | |
|---|---|---|---|---|---|---|
| Cmpd | Starting (S-B-U1) | Compound # | Net | Theo. | MS (ESI) | Particle |
| # | Product (S-B-U-H[(D)]) | | charge | MW | m/n Found | size (nm) |
| 242 | Compound 150 | Compound 58 | 0 | 4681.3 | 1172.1 | 25.7 |
| | (Mannose-ethyl)₄-PEG₂₄-(N3-DBCO)-Ahx-(F′)10 | | | | (M/4 + H)⁺ | |
| 243 | N3-PEG₂₄-KKKKKKKK-NH₂ | Compound 58 | +8 | 4235.8 | 1059.8 | 19.9 |
| | KKKKKKKK-PEG₂₄-(N₃-DBCO)-Ahx-F′10 | | | | (M/4 + H)⁺ | |
| 244 | N3-PEG₂₄-DDDDDDD-COOH | Compound 58 | −8 | 4116.1 | 1372.7 | 19.6 |
| | DDDDDDD-PEG₂₄-(N₃-DBCO)-Ahx-F′10 | | | | (M/3)⁺ | |
| 245 | KKKKKKKKK-PEG₂₄-X | Compound 136 | +10 | 4509.2 | 1128.4 | 12.8 |
| | KKKKKKKKK-PEG₂₄-(N3-DBCO)-Ahx-2B₃W₂ | | | | (M/4 + H)⁺ | |
| 246 | N₃-PEG₂₄-DDDDDDDDD-NH₂ | Compound 136 | −10 | 4264.3 | 1067.4 | 12.0 |
| | DDDDDDDDD-PEG₂₄-N₃-(N3-DBCO)-Ahx-2B₃W₂ | | | | (M/4 + H)⁺ | |
| 247 | OOOOOOOOO-PEG₂₄-X | Compound 136 | +10 | 4383.0 | 1096.9 | 13.5 |
| | OOOOOOOOO-PEG₂₄-X-(N3-DBCO)-Ahx-2B₃W₂ | | | | (M/4)⁺ | |
| 248 | kkkkkkkkk-PEG₂₄-X | Compound 136 | +10 | 4509.4 | 1128.5 | 12.8 |

| | |
|---|---|
| kkkkkkkkk-PEG₂₄-(N3-DBCO)-Ahx-2B₃W₂ | (M/4 + H)⁺ |

Single letter abbreviations are used for amino acid sequences in the above table; X = azidolysine; O = Ornithine; and k = d-lysine. Peptide-based starting materials were manufactured by solid-phase peptide synthesis by Genscript (Piscataway, NJ). Peptide-based starting material sequences are written from N- to C-terminus, and C-terminal -NH₂ and -COOH indicate that the peptides are terminated with either amide or carboxylic acid groups, respectively. Unless otherwise specified, any C-terminal X, azidolysine, is terminated with an amide and is implicit in the sequences (i.e., not shown). Net charge is the predicted net charge of the amphiphile in aqueous buffer at pH 7.4.

### Additional Peptide Antigen Conjugates of Formula [S]-[E1]-A-[E2]-U-H-[D]

Compounds 249-256 (Table 11) were synthesized in a similar manner as for those described in Table 8.

TABLE 11
Additional peptide antigen conjugates of formula [S]-[E1]-A-[E2]-U-H-[D].

| Cmpd | | | Theo | Found |
|---|---|---|---|---|
| # | [S]-[E1]-A-[E2]-U1 | U2-H-D | MW | MW |
| 249 | KKKKKKKKKVRGIPVHLELASMTNMELMSSIVHQQV | Compound 136 | 7168.9 | 1793.1 |
| | FPTSPVZX (SEQ ID NO: 429) | | | (M/4 + H)⁺ |
| | KKKKKKKKKVRGIPVHLELASMTNMELMSSIVHQQVFPTSPVZX(N3- | | | |
| | DBCO)-Ahx-2B3W2) | | | |
| 250 | KKKKKKKVRGRVLELFRAAQLANDVVLQIMELCGAT | Compound 136 | 6889.5 | 1723.5 |
| | RSPVZX (SEQ ID NO: 430) | | | (M/4 + H)⁺ |
| | KKKKKKKVRGRVLELFRAAQLANDVVLQIMELCGATRSPVZX(N3- | | | |
| | DBCO)-Ahx-2B3W2 | | | |
| 251 | KKKKKKKKVRKARDETAALLNSAVLGAAPLFVPPAD | Compound 136 | 6410.2 | 1603.7 |
| | SPVZX (SEQ ID NO: 431) | | | (M/4 + H)⁺ |
| | KKKKKKKKVRKARDETAALLNSAVLGAAPLFVPPADSPVZX(N3- | | | |
| | DBCO)-Ahx-2B3W2 | | | |
| 252 | KKKKKKKKKVRDFTGSNGDPSSPYSLHYLSPTGVNEY | Compound 136 | 6863.5 | 1716.8 |
| | SPVZX (SEQ ID NO: 432) | | | (M/4 + H)⁺ |
| | KKKKKKKKKVRDFTGSNGDPSSPYSLHYLSPTGVNEYSPVZX(N3- | | | |
| | DBCO)-Ahx-2B3W2 | | | |
| 253 | KKKKKKKKKVRGIPVHLELASMTNMELMSSIVHQQV | Compound 62 | 6461.4 | 1616.2 |
| | FPTSPVZX (SEQ ID NO: 429) | | | (M/4 + H)⁺ |
| | KKKKKKKKKVRGIPVHLELASMTNMELMSSIVHQQVFPTSPVZX(N3- | | | |
| | DBCO)-Ahx-W5 | | | |
| 254 | KKKKKKKVRGRVLELFRAAQLANDVVLQIMELCGAT | Compound 62 | 6182.0 | 1546.1 |
| | RSPVZX (SEQ ID NO: 430) | | | (M/4 + H)⁺ |
| | KKKKKKKVRGRVLELFRAAQLANDVVLQIMELCGATRSPVZX(N3- | | | |
| | DBCO)-Ahx-W5 | | | |
| 255 | KKKKKKKVRKARDETAALLNSAVLGAAPLFVPPADS | Compound 62 | 5702.7 | 1426.3 |
| | PVZX (SEQ ID NO: 433) | | | (M/4 + H)⁺ |
| | KKKKKKKKVRKARDETAALLNSAVLGAAPLFVPPADSPVZX(N3- | | | |
| | DBCO)-Ahx-W5 | | | |
| 256 | KKKKKKKKKVRDFTGSNGDPSSPYSLHYLSPTGVNEY | Compound 62 | 6156.0 | 1539.6 |
| | SPVZX (SEQ ID NO: 432) | | | (M/4 + H)⁺ |
| | KKKKKKKKKVRDFTGSNGDPSSPYSLHYLSPTGVNEYSPVZX(N3- | | | |
| | DBCO)-Ahx-W5 | | | |

Single letter abbreviations are used for amino acid sequences in the above table; X = azidolysine and Z = citrulline. Peptide-based starting materials were manufactured by solid-phase peptide synthesis by Genscript (Piscataway, NJ). Unless otherwise specified, any C-terminal X, azidolysine, is terminated with an amide and is implicit in the sequences (i.e., not shown).

### Hydrodynamic Behavior of Nanoparticles Comprising Amphiphilic Carriers

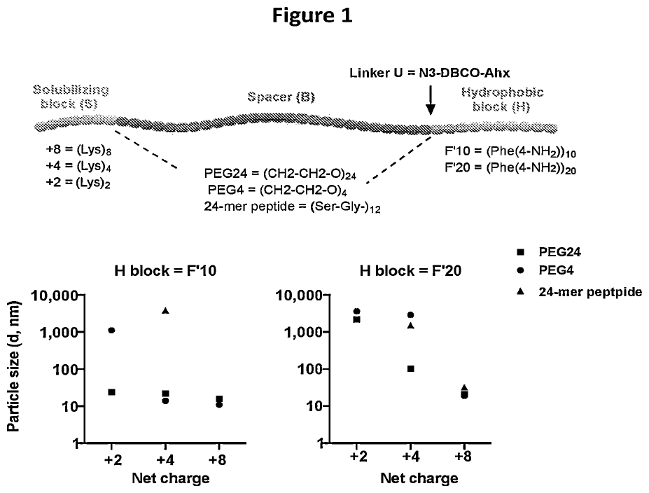

Our initial studies focused on evaluating how the composition of the spacer (B) and net charge of the amphiphile impact the capacity of linear amphiphiles of formula S-B-U-H to assemble into nanoparticle micelles (FIG. 1). As expected, increasing net charge from +2 to +8 led to increased propensity of amphiphiles to assemble into nanoparticle micelles (FIG. 1) independent of the hydrophobic block length or spacer composition. While amphiphiles with spacers comprise either PEG4, PEG24 or 24 amino acids all formed nanoparticle micelles with net charge of +8, those with PEG4- or peptide-based spacers, but not PEG24, tended to aggregate with net charge of +2 or lower (FIG. 1). suggesting that intermediate length PEG spacers may be generally preferred over amino acid or short PEG-based spacers (B).

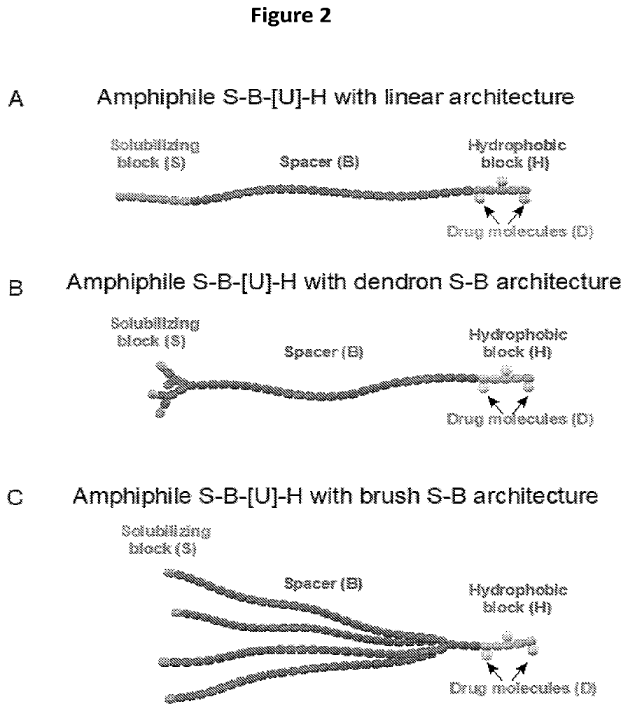

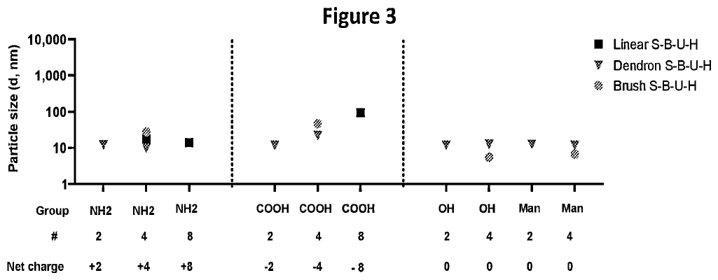

Our next studies investigated how the composition and architecture of the solubilizing block impact the capacity of linear amphiphiles of formula S-B-U-H to assemble into nanoparticle micelles. Amphiphiles with either linear, dendron or brush architecture having either amine, carboxylic acid, hydroxyl or mannose based solubilizing groups were evaluated (FIGS. 2 and 3). Notably, while linear amphiphiles typically required net charge greater than +4 or less than −4 to ensure stable nanoparticle micellization, dendron amphiphiles formed stable nanoparticle micelles with neutral charge. In contrast to the linear and dendron structures, brush-based amphiphiles exhibited greater particle size variability that was independent of net charge. Altogether, these data indicate that the amphiphile architecture has a major impact on hydrodynamic behavior as well as the requirements (e.g., net charge required) for micellization.

### Hydrodynamic Behavior of Nanoparticles Comprising Amphiphilic Carriers and Peptide Antigen Conjugates

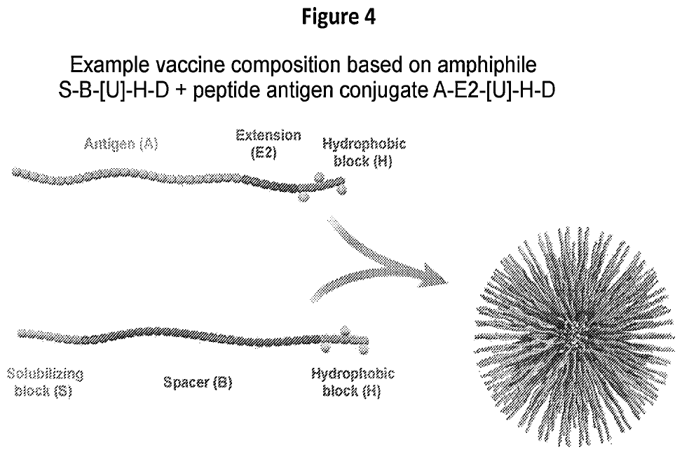

Peptide antigens have diverse physical and chemical properties that can lead to variable biological activity. To standardize peptide antigen delivery into nanoparticles of uniform size and composition as a means to enable more predictable biological activity, the inventors of the present disclosure developed vaccines comprising so-called “mosaic” nanoparticles comprising one or more peptide antigen conjugates and an amphiphilic carrier (FIG. 4). The core of the amphiphilic carrier (e.g., S-[B]-[U]-H-[D]) solubilizes the hydrophobic block present on the peptide antigen conjugates (thereby incorporating the peptide antigen conjugated into the particles), while the solubilizing block of the amphiphile stabilizes the resulting nanoparticle micelle and preventing aggregation that otherwise may occur with peptide antigen conjugates alone. The benefit of this approach is that it enables a simple means of incorporating multiple different antigens (as peptide antigen conjugates) into nanoparticles of uniform composition.

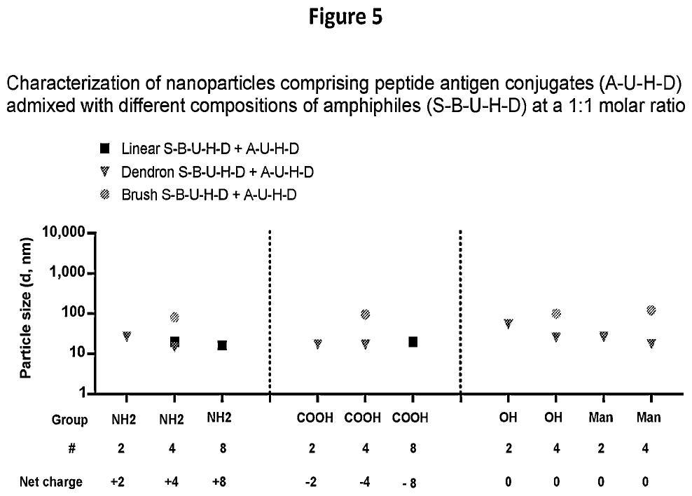

While the above studies established how solubilizing block composition and architecture impacted nanoparticle micellization by amphiphiles alone, it was previously unknown how such parameters would impact hydrodynamic behavior of compositions comprising both peptide antigen conjugates and amphiphiles. Therefore, we evaluated the hydrodynamic behavior of nanoparticles comprising a peptide antigen conjugate with varying composition of the amphiphile (FIG. 5) at a 1:1 molar ratio of peptide antigen conjugate to amphiphile. Similar to the results observed with the amphiphile alone, nanoparticles comprising amphiphiles with dendron architecture formed stable nanoparticles with neutral net charge, whereas those with linear or brush architecture tended to form larger particles, possibly aggregates, at charge greater than −4 or less than +4. These results established that amphiphiles with dendron architecture admixed with peptide antigen conjugates promote mosaic nanoparticle micelles independent of charge.

### Example 3: Compositions of Vaccines Based on Mosaic Nanoparticles Comprising an Amphiphile and One or More Peptide Antigen Conjugates

### Impact of Amphiphilic Carrier Composition on Hydrodynamic Behavior and Immunogenicity of Vaccines for Treating or Preventing Cancer

A non-limiting example of a vaccine for cancer treatment comprises an amphiphile and at least one peptide antigen conjugate, wherein the peptide antigen conjugate comprises an antigen (A) selected from tumor antigens, such as neoantigens. In certain embodiments of vaccines for cancer treatment, the vaccine is prepared by combining the amphiphile with one or more peptide antigen conjugates at a specified molar ratio of total amphiphile to total peptide antigen conjugate in a polar aprotic solvent, such as DMSO, DMF or DMAC; aqueous buffer is then added to the mixture followed by gentle mixing to yield an aqueous formulation of mosaic nanoparticles comprising the amphiphile and one or more peptide antigen conjugates.

Table 12 provides examples of cancer vaccines comprising amphiphiles of formula S-B-U-H-D with either positive charge and linear architecture (Compounds 169 and 172), negative charge and linear architecture (Compounds 174 and 175) or neutral charge and dendron (or “cone”) architecture (Compounds 162 and 163), wherein in each case the amphiphile was formulated with 4 peptide antigen conjugates at a 1:1 molar ratio of amphiphile to peptide antigen conjugate. For example, for group 2, 48 μL of a 5 mM DMSO solution of the amphiphile Compound 174 (240 nmol, 4 eq) was added to a 2 mL polypropene vial, followed by the addition of 12 μL of 5 mM DMSO solutions of peptide antigen conjugates Compound 198 (60 nmol, 1 eq), Compound 199 (60 nmol, 1 eq), Compound 202 (60 nmol, 1 eq), and Compound 224 (60 nmol, 1 eq), and 5.76 uL of a 500 mM DMSO solution of Tris base (2880 nmol, 48 eq). The solution was vortexed and then 1098.2 uL of PBS was added followed by gentle mixing to yield an aqueous vaccine formulation comprising mosaic nanoparticles further comprising amphiphiles and peptide antigen conjugates. The hydrodynamic behavior of the nanoparticles was characterized by turbidity and DLS measurements and is summarized in Table 12.

TABLE 12
Cancer vaccines comprising positive, negative
or neutral (mannose) nanoparticles

| | | | | Parti- |
|---|---|---|---|---|
| Group | | | Turbidity | cle |
| # | S-B-U-H-[D] | A-U-H-[D] | (490 nm) | size (nm) |
| 1 | Compound 169 | Compound 198 | 0.041 | 23 |
| | | Compound 199 | | |
| | | Compound 202 | | |
| | | Compound 224 | | |
| 2 | Compound | Compound 198 | 0.078 | 40.72 |
| | 174 + | Compound 199 | | |
| | Tris base | Compound 202 | | |
| | | Compound 224 | | |
| 3 | Compound 162 | Compound 198 | 0.04 | 26.36 |
| | | Compound 199 | | |
| | | Compound 202 | | |
| | | Compound 224 | | |
| 4 | Compound 172 | Compound 220 | 0.04 | 19.74 |
| | | Compound 221 | | |
| | | Compound 222 | | |
| | | Compound 223 | | |
| 5 | Compound | Compound 220 | 0.04 | 22.63 |
| | 175 + | Compound 221 | | |
| | Tris base | Compound 222 | | |
| | | Compound 223 | | |
| 6 | Compound 163 | Compound 220 | 0.04 | 15.37 |
| | | Compound 221 | | |
| | | Compound 222 | | |
| | | Compound 223 | | |

A major advantage of amphiphilic carriers having dendron architecture is that such amphiphiles enable nanoparticle micellization independent of charge, thereby enabling the use of a broader range of chemical compositions of solubilizing blocks than is possible the linear amphiphiles described herein. For instance, while particles with high net positive charge are highly immunogenic, such compositions can lead to toxicity, particularly by the intravenous route. Therefore, compositions with neutral or negative charge may be preferred for certain applications.

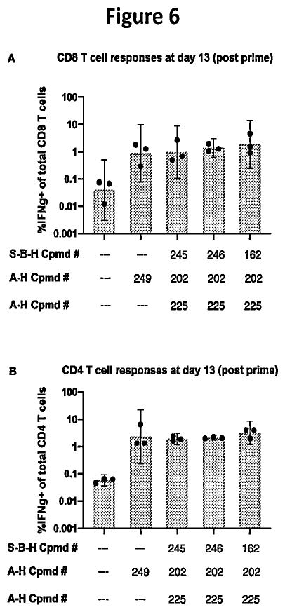

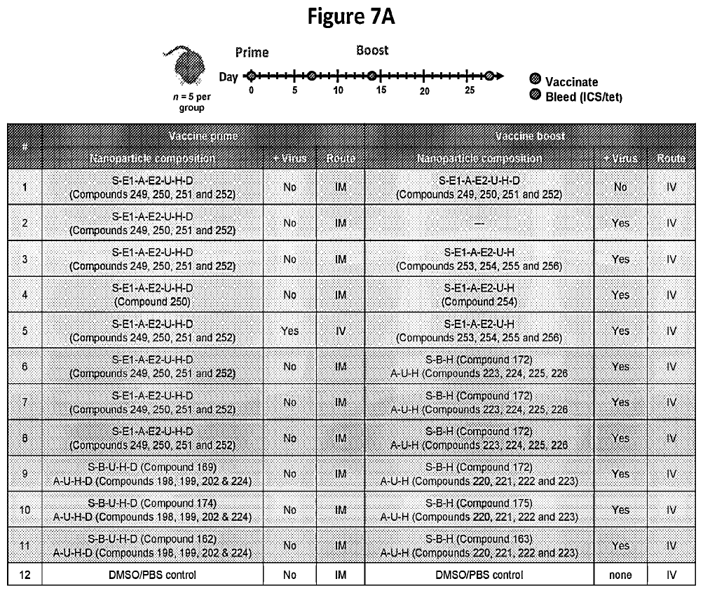

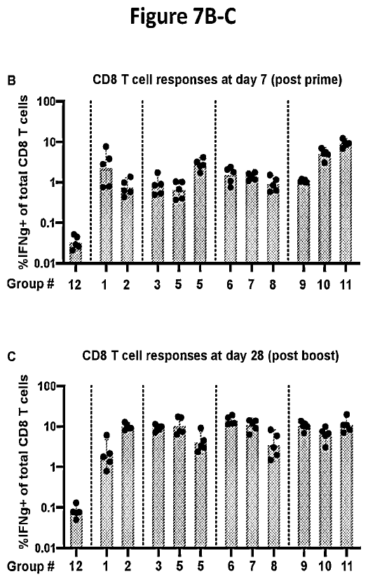

To assess how solubilizing block composition and net charge impacts immune responses, mice were immunized with vaccines comprising a peptide antigen conjugate and an amphiphile of formula S-B-H with either linear architecture and positive charge (compound 245), linear architecture and negative charge (compound 246) or dendron architecture and mannose based solubilizing groups having net neutral charge (compound 162; FIG. 6). Notably, the vaccine composition with the dendron architecture (compound 162) led to higher magnitude CD8 T cell responses and lower toxicity (not shown) as compared with vaccine compositions having linear amphiphiles with either positive or negative charge (FIG. 6). Importantly, these trends were reproduced in an independent study, with a vaccine composition comprising an amphiphile with dendron architecture and mannose-based solubilizing groups having net neutral charge (compound 162) leading to the higher magnitude CD8 T cell responses as compared with the vaccine compositions comprising amphiphiles with linear architecture and either net positive or net neutral charge (FIG. 7a-b). Though, after a boost immunization comprising vaccinia virus as a form of a biological adjuvant co-administered with different nanoparticle vaccine compositions, the differences between groups were largely diminished (FIG. 7C).

### Impact of Amphiphilic Carrier Composition on the Hydrodynamic Behavior of Vaccines for Inducing Tolerance

A similar process was used to prepare compositions of vaccines for inducing tolerance as was used to prepare vaccines for cancer treatment. Table 13 provides examples of vaccines for inducing tolerance comprising amphiphiles of formula S-B-U-H with either positive charge and linear architecture (Compound 172), negative charge and linear architecture (Compound 175), neutral charge (saccharide solubilizing groups) and dendron (or “cone”) architecture (Compound 163) or negative charge (from a saccharide-based solubilizing block) and linear architecture (Compound 177), wherein in each case the amphiphile was formulated with a peptide antigen conjugate comprising an autoantigen (“MOG”) and a drug (D), Rapamycin, at a 1:1:1 molar ratio of amphiphile to peptide antigen conjugate to drug. Additionally, 1.5 molar equivalents of Tris was added to formulations comprising amphiphiles further comprising carboxylic acids.

For example, for group 2 in Table 13, 5.65 uL of a 13.28 mM DMSO solution of Compound 175 (75 nmol, 1 eq) and 8.91 uL of 8.42 mM DMSO solution of Compound 218 (75 nmol, 1 eq) were added to a 2.0 mL polypropylene tube and then the DMSO was removed under vacuum. To this tube was added 9 uL DMSO, 3.75 uL of a 20 mM DMSO solution of Rapamycin (75 nmol, 1 eq) and 2.25 uL of a 400 mM DMSO solution of Tris (900 nmol, 12 eq). The solution was vortexed to yield a mixture wherein Compound 218 was at 5 mM in DMSO. The vial was vortexed and then 2,385 uL of PBS buffer was added to yield an aqueous vaccine formulation comprising mosaic nanoparticles further comprising amphiphiles, peptide antigen conjugates and drug molecules. The hydrodynamic behavior of the nanoparticles was characterized by turbidity and DLS measurements and is summarized in Table 13.

TABLE 13
Vaccines for inducing tolerance comprising positive,
negative or neutral amphiphiles, a peptide antigen
conjugate and a drug (i.e., Rapamycin).

| | | | | Tur- | Parti- |
|---|---|---|---|---|---|
| | | | | bidity | cle |
| Group | | | | (490 | Size |
| # | S-B-U-H | A-U-H | Drug | nm) | (nm) |
| 1 | Compound 172 | Compound 218 | Rapamycin | 0.039 | 18.22 |
| 2 | Compound | Compound 218 | Rapamycin | 0.049 | 59.81 |
| | 175 + Tris | | | | |
| 3 | Compound 163 | Compound 218 | Rapamycin | 0.039 | 16.8 |
| 4 | Compound 177 | Compound 218 | Rapamycin | 0.043 | 17.89 |

### Impact of Amphiphilic Carrier Composition on the Hydrodynamic Behavior and Immunogenicity of Vaccines for Inducing Antibodies

A similar process was used to prepare compositions of vaccines for inducing antibodies as was used to prepare vaccines for cancer treatment. Table 14 provides examples of vaccines for inducing antibodies comprising amphiphiles of formula S-B-U-H with either positive charge and linear architecture having a PEG24 spacer (Compound 169), positive charge and linear architecture have a PEG4 spacer (Compound 167), or positive charge and brush architecture (Compound 185), wherein in each case the amphiphile was formulated with one or more peptide antigen conjugates comprising B cell epitopes and a peptide antigen conjugate comprising a universal CD4 T cell epitope at a 1:1 molar ratio of amphiphile to total peptide antigen conjugate. Note that the molar ratio of peptide antigen conjugates comprising B cell epitopes to peptide conjugates comprising a universal CD4 T cell epitope was 4:1.

For example, for group 1, 7.2 uL of a 5 mM DMSO stock of Compound 211 (36 nmol, 1 eq) was added to a 2 mL polypropylene tube. 8 μL of a 5.4 mM DMSO stock of Compound 169 (43.2 nmol, 1.2 eq) was added, followed by 1.76 uL of a 4.1 mM DMSO stock of Compound 225 (7.2 nmol, 0.2 eq). The mixture was diluted with 150 μL of DMSO, sterile filtered and then DMSO was removed under vacuum. The dried material was resuspended in 7.2 uL of sterile DMSO to yield a solution with Compound 211 at 5 mM in DMSO. The vial was vortexed and 712.8 uL of PBS was added to yield an aqueous vaccine formulation comprising mosaic nanoparticles further comprising amphiphiles and peptide antigen conjugates. The hydrodynamic behavior of the nanoparticles was characterized by turbidity and DLS measurements and is summarized in Table 14 below.

TABLE 14
Vaccines for inducing antibody responses comprising amphiphiles
and one or more different peptide antigen conjugates each
comprising B cell epitopes and/or T cell epitopes.

| | | Peptide antigen | Peptide antigen | | |
|---|---|---|---|---|---|
| | | conjugates | conjugate | Tur- | Parti- |
| | | comprising | comprising | bidity | cle |
| Group | | B cell | CD4 T cell | (490 | size |
| # | S-B-U-H-D | epitopes | epitope | nm) | (nm) |
| 1 | Compound 169 | Compound 211 | Compound 225 | 0.04 | 13.45 |
| 2 | Compound 169 | Compound 207 | Compound 225 | 0.04 | 12.37 |
| 3 | Compound 169 | Compound 208 | Compound 225 | 0.041 | 11.51 |
| 4 | Compound 167 | Compound 211 | Compound 225 | 0.039 | 12.21 |
| 5 | Compound 167 | Compound 207 | Compound 225 | 0.04 | 11.4 |
| 6 | Compound 167 | Compound 208 | Compound 225 | 0.039 | 10.94 |
| 7 | Compound 167 | Compound 207 | Compound 225 | 0.04 | 15.88 |
| | | Compound 212 | | | |
| | | Compound 216 | | | |
| 8 | Compound 169 | Compound 207 | Compound 225 | 0.041 | 16.45 |
| | | Compound 212 | | | |
| | | Compound 216 | | | |
| 9 | Compound 185 | Compound 207 | Compound 225 | 0.042 | 97.43 |
| | | Compound 212 | | | |
| | | Compound 216 | | | |
| 10 | Compound 167 | Compound 208 | Compound 225 | 0.045 | 12.79 |
| | | Compound 213 | | | |
| | | Compound 217 | | | |
| 11 | Compound 169 | Compound 208 | Compound 225 | 0.039 | 12.23 |
| | | Compound 213 | | | |
| | | Compound 217 | | | |
| 12 | None | Compound 208 | Compound 225 | 0.041 | 13.01 |
| | | Compound 213 | | | |
| | | Compound 217 | | | |

The above formulation process was used to evaluate the hydrodynamic behavior of particles formed by admixing one or more peptide antigen conjugates with amphiphilic carriers of varying composition and architecture. In contrast to cancer vaccines or vaccines for inducing tolerance that are focused on generating antigen-specific T cells responses against linear peptides that are processed and presented by antigen-presenting cells, antibody vaccines must also ensure that B cell epitopes are solvent exposed and accessible to B cell receptors on the surface of the particles (e.g., nanoparticle micelles formed by the one or more peptide antigen conjugates and amphiphilic carrier). Therefore, in addition to evaluating the impact of the amphiphilic carrier composition, we also evaluated how varying length of the extension (E1 or E2) between the peptide antigen (A) and hydrophobic block (H), as well as the spacer length (B) between the solubilizing group and amphiphilic carrier impacted particle stability as well as antibody responses generated against the peptide antigen. Our rationale was that providing the antigen (A) linked to an extension E1 (or E2) that is greater than or equal to length of B would ensure that the antigen is adequately solvent exposed and accessible to B cells, whereas E1 (or E2) less than B may leader to lower magnitude responses due to inadequate solvent exposure. However, solvent exposure of peptide antigen (A) without a solubilizing block (S) (e.g., A-[E2]-[U]-H[D] or H-[D]-[U]-[E1]-A) has the potential to lead to particle instability.

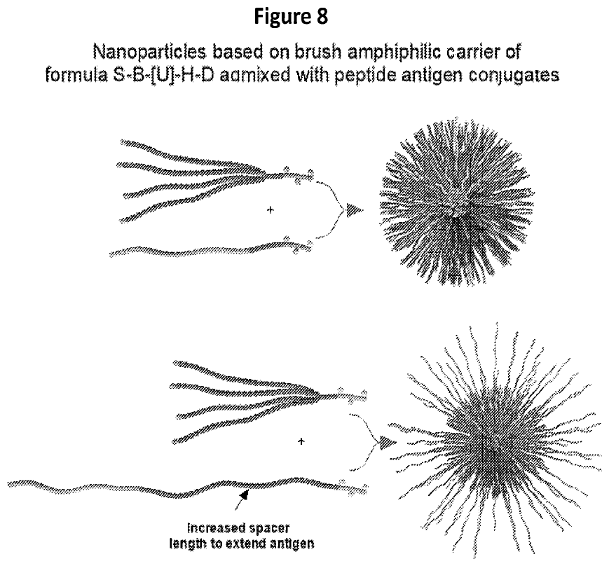

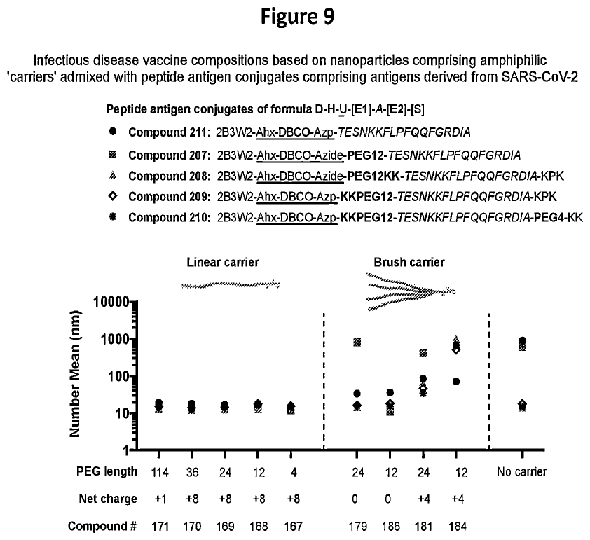

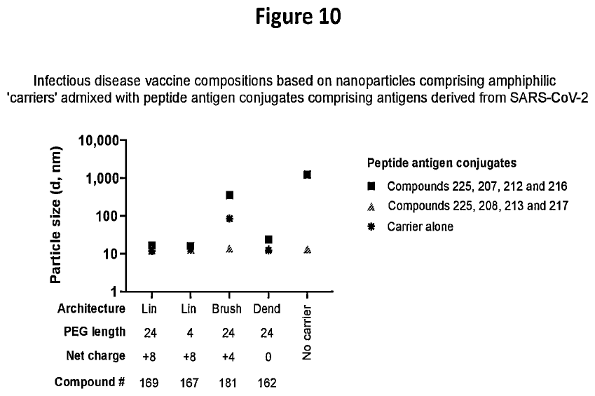

Our next studies evaluated hydrodynamic stability of different B cell vaccine compositions with varying amphiphilic carrier composition and architecture, as well as varying lengths of peptide antigen conjugate extension (see: FIG. 8) and varying length of spacer (B) of the amphiphilic carrier. Consistent with earlier findings (see: FIG. 5), vaccine compositions comprising amphiphilic carriers with dendron architecture or linear architecture and high net charge (or linear architecture and PEG spacer (B) length at least greater than 36 units) generally formed stable nanoparticle micelles, whereas those amphiphilic carriers with brush architecture generally assembled into submicron particles (FIGS. 9 and 10). Additional notable findings were that nanoparticle micelles were stable even when the peptide antigen conjugate extension E1 was greater than, equal to or less than the length of the spacer B present on the amphiphilic carrier, suggesting that projection of the antigen had minimal impact on hydrodynamic behavior, and introduction of short, peptide-based solubilizing blocks on the peptide antigen conjugates (i.e., Lys-Pro-Lys) enabled the peptide antigen conjugates to assemble into stable nanoparticle micelles without the presence of the amphiphilic carrier (FIG. 9).

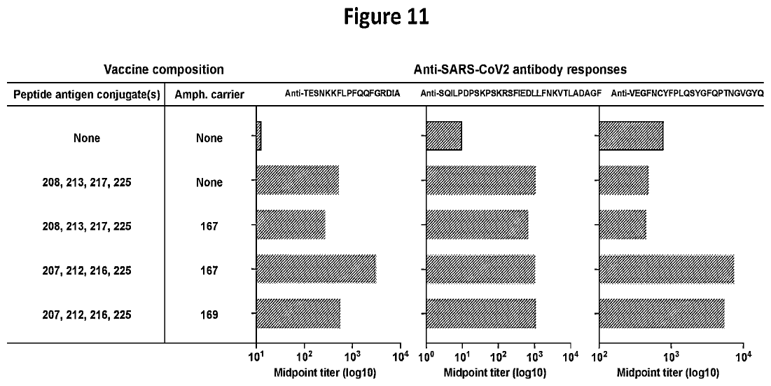

Though the peptide antigen conjugates comprising solubilizing blocks formed stable nanoparticles without carrier, the presence of a terminal solubilizing block adjacent to the peptide antigen of the peptide antigen conjugate was deleterious to antibody responses (FIG. 11). Accordingly, vaccine compositions comprising four peptide antigen conjugates without solubilizing blocks (compounds 207, 212, 216 and 225) and an amphiphilic carrier (compound 167 or 169) led to higher magnitude antibody responses against the peptide antigen (A) than vaccine compositions comprising peptide antigen conjugates further comprising solubilizing groups (compounds 208, 213 and 217; FIG. 11). These results suggest that, while solubilizing blocks are critical to stability of nanoparticles comprising peptide antigen conjugates, it is preferential to decouple the solubilizing block from the peptide antigen through the use of amphiphilic carriers as a means to reduce interference with antibody responses generated against the peptide antigen.

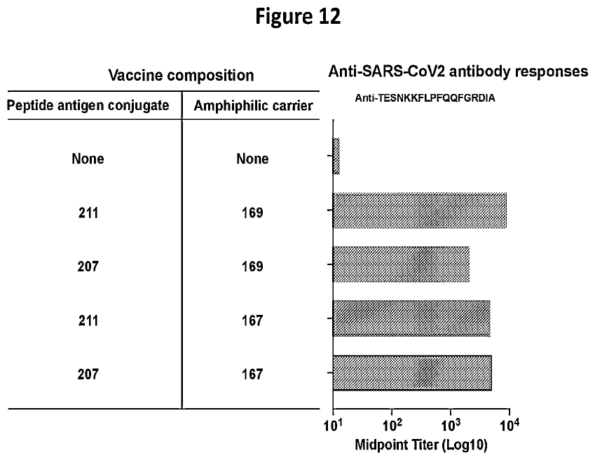

A final notable finding was that peptide antigen conjugates with an extension (E1 or E2) of greater than or equal length than the spacer (B) of the amphiphilic carrier generally led to higher magnitude antibody responses (e.g., compare groups with amphiphilic carrier compound 167 (PEG4) versus compound 169 PEG) (FIG. 12); however, the rigidity of the extension was also found to have a substantial impact on antibody responses as peptide antigen conjugates having rigid peptide-based extensions generally induced higher magnitude antibody responses than those with PEG-based extensions (data not shown).

### Example 4: Peptide Antigen Conjugates with Non-Native Amino Acids

The amino acids cysteine and methionine are prone to oxidation, which can present challenges to manufacturing peptide-based therapeutics, including vaccines, that comprise peptides with one or more cysteine and/or methionine residues. For instance, the inventors of the present disclosure observed that peptide antigen conjugates comprising one or more cysteine residues were prone to forming dimers or multimers (due to intermolecular disulfide bond formation) upon storage in certain organic solvents (e.g., DMSO) or in aqueous solutions under ambient air. Oxidation resulting in intermolecular disulfide bond formation results in reduced purity and can potentially impact biological activity. Therefore, strategies for overcoming the challenges of cysteine and methionine oxidation are needed.

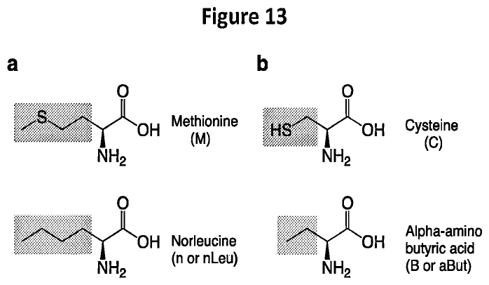

As a means to overcome the challenges of cysteine and methionine oxidation, the inventors of the present disclosure evaluated the permissibility of substituting naturally occurring cysteine (Cys) and methionine (Met) residues of peptide antigens with amino acids of similar structure, e.g., alpha aminobutyric acid (aBut) and norleucine (nLeu), respectively, that are note prone to oxidation (FIG. 13). It was unknown a priori how substituting Cys and Met residues with aBut and nLeu, respectively, would affect the capacity of the resulting non-natural antigens to induce T cells in vivo that are cross-reactive for the natural (or “native”) antigens. Therefore, a series of antigens (Table 15) with one or more cysteine and/or methionine residues were synthesized and screened in vivo for the capacity to elicit T cells that recognize both the non-natural vaccine peptide antigen, as well as the naturally occurring (“native”) peptide antigen.

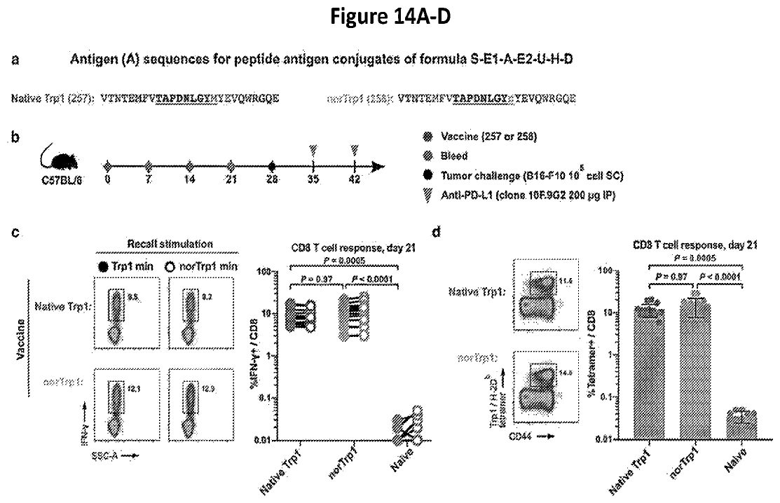

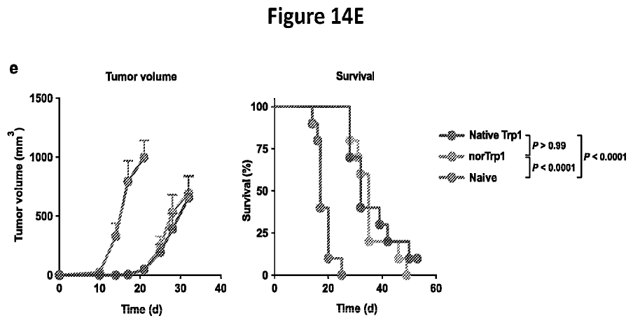

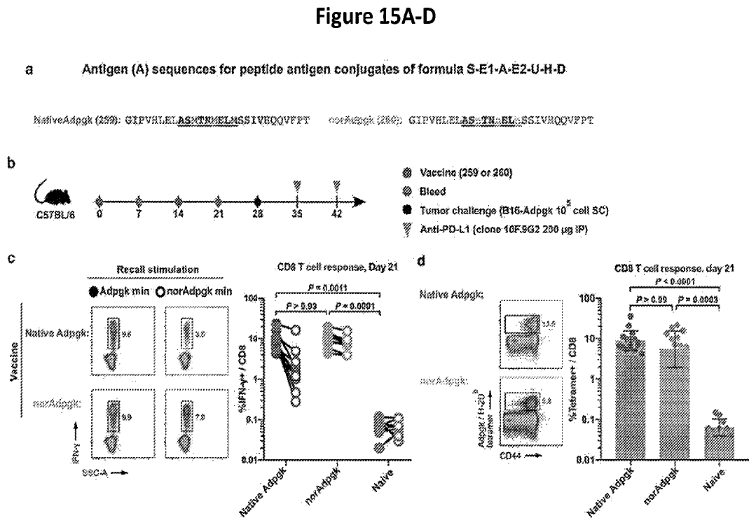

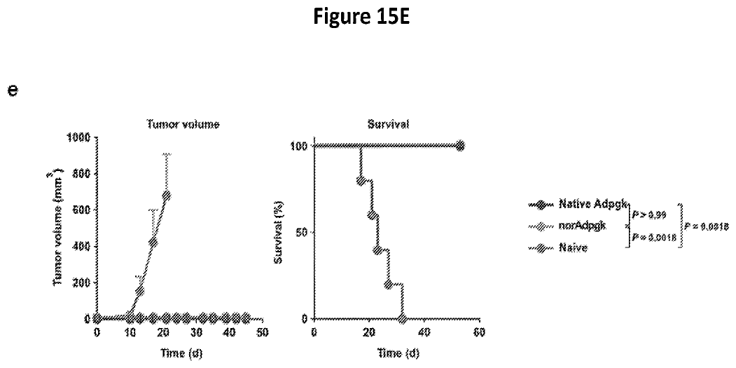

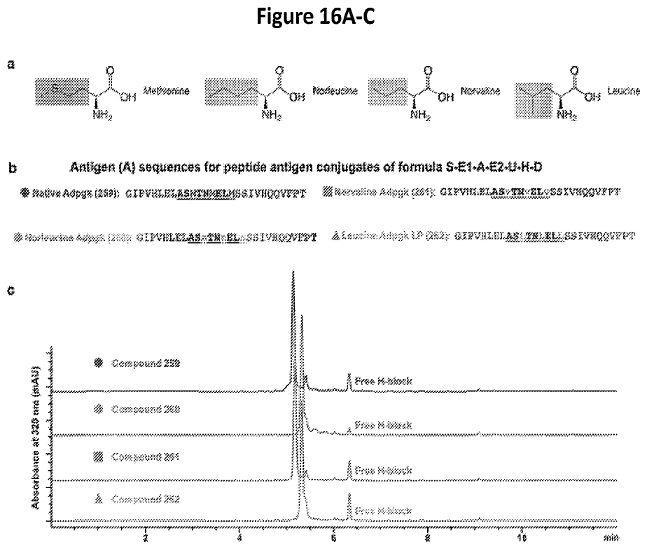

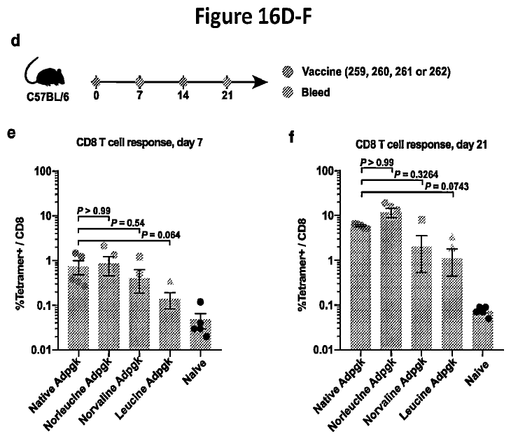

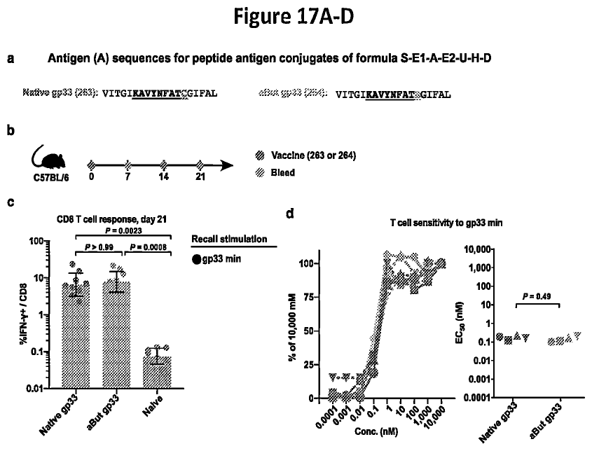

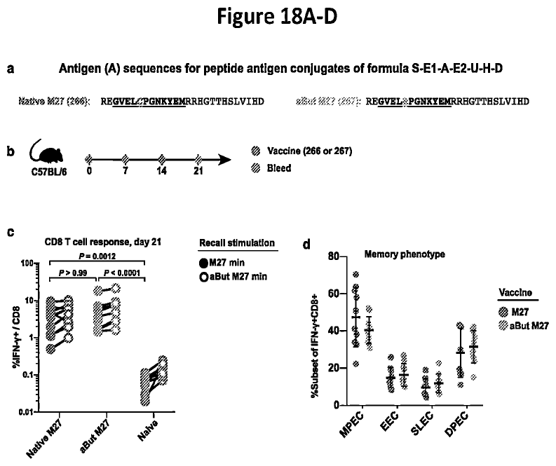

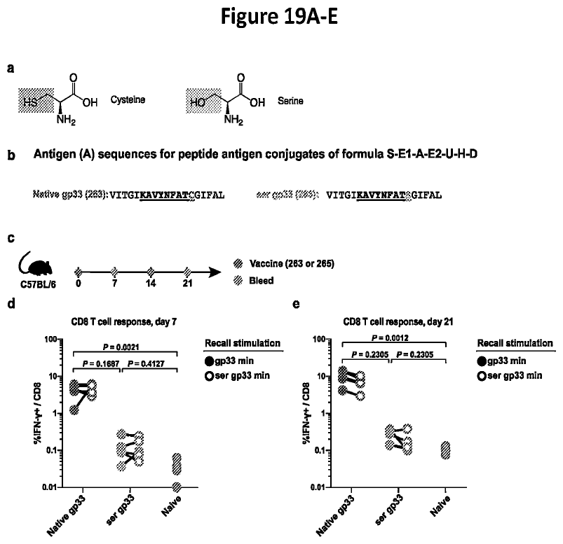

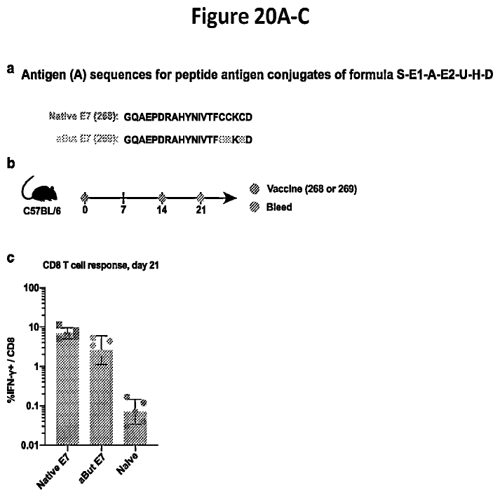

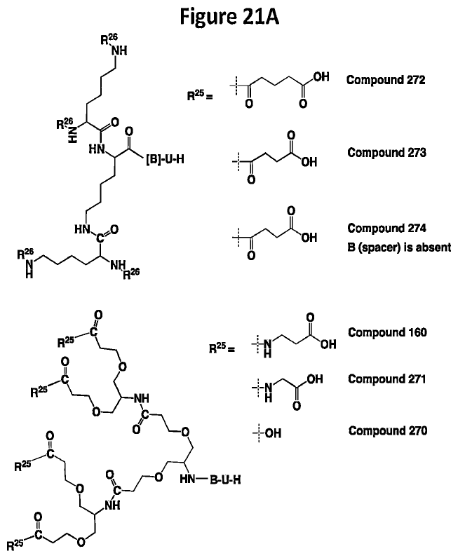

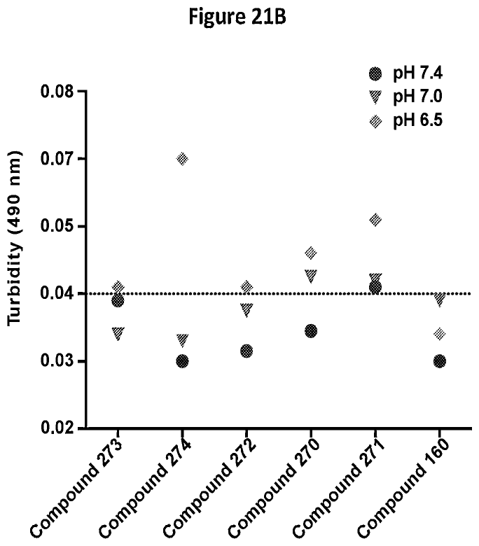

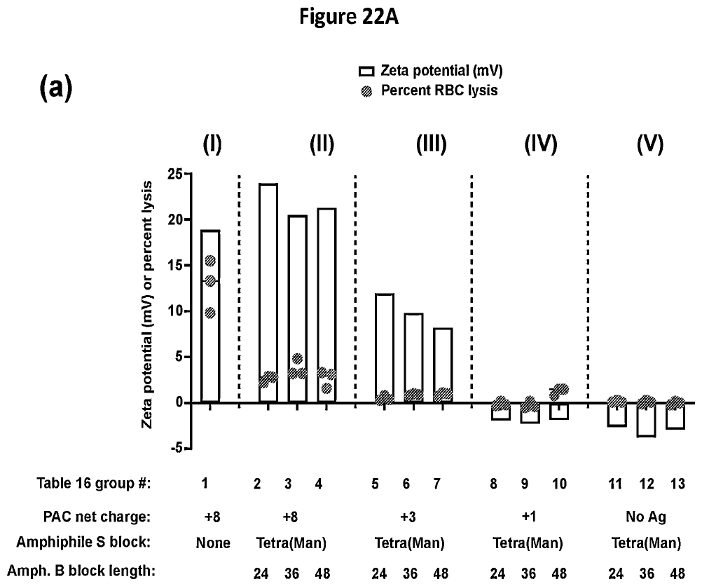

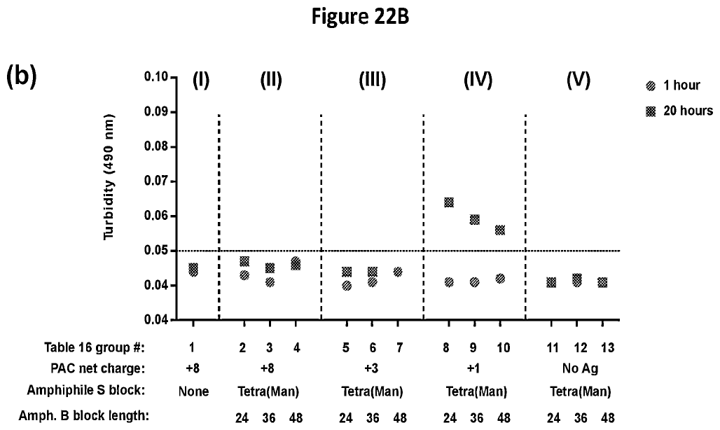

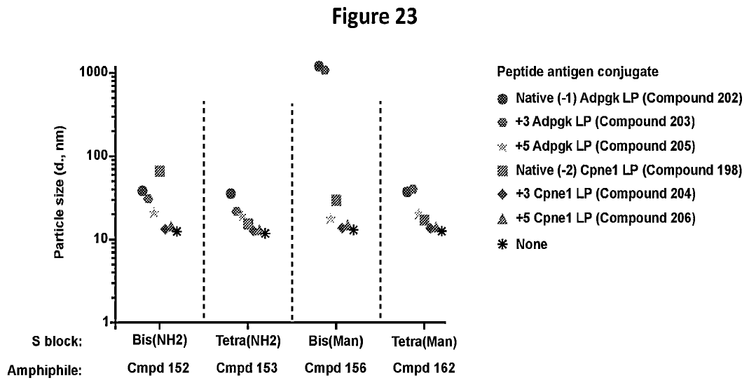

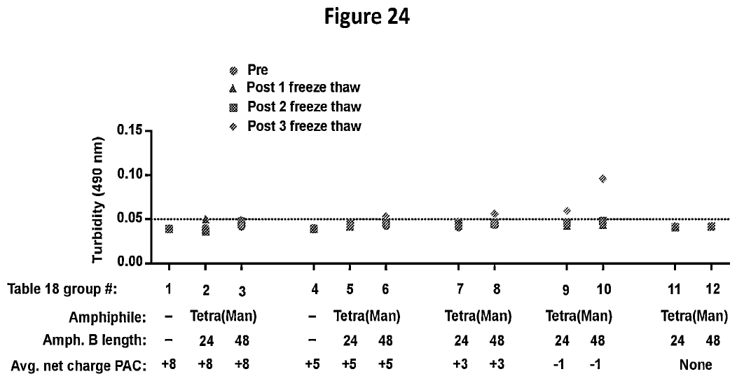

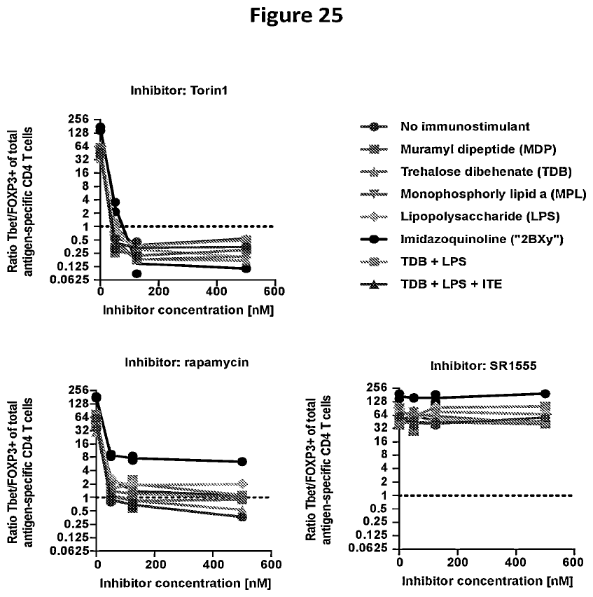

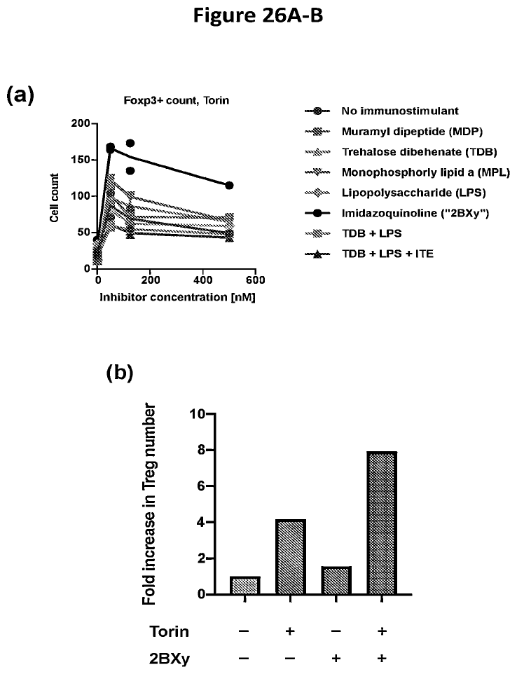

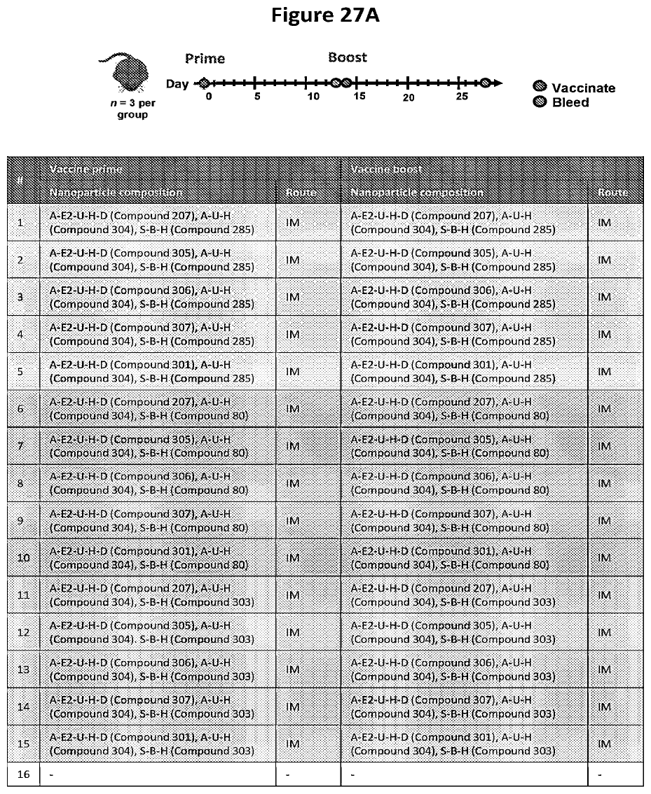

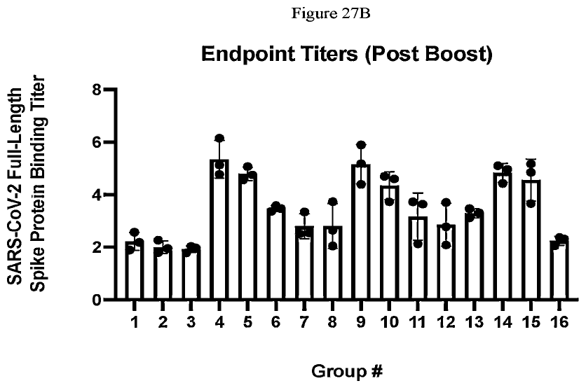

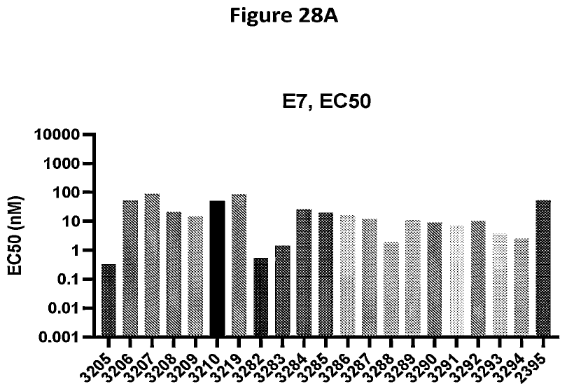

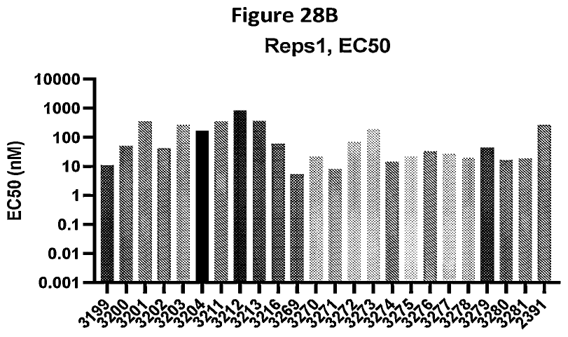

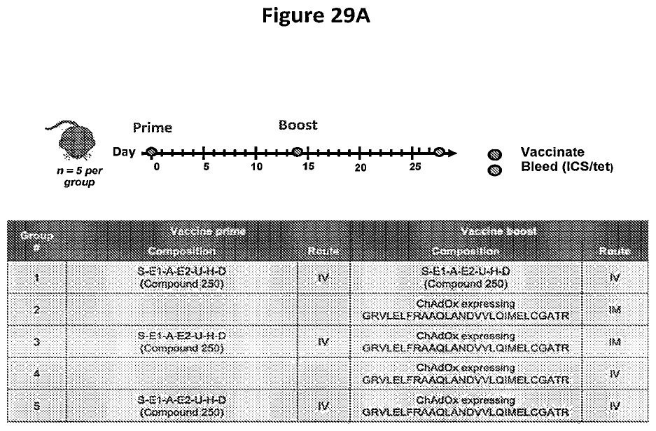

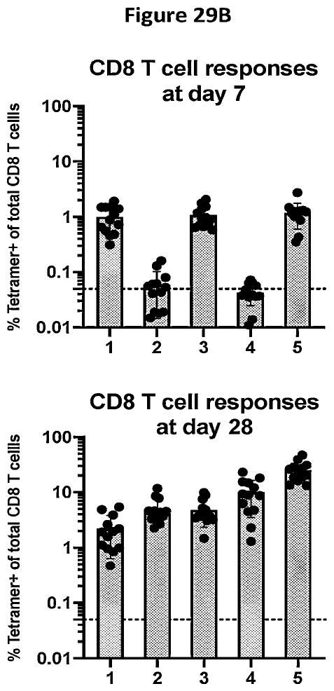

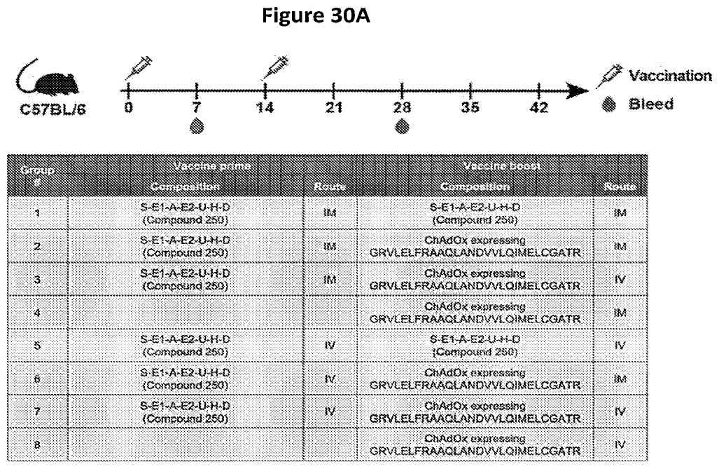

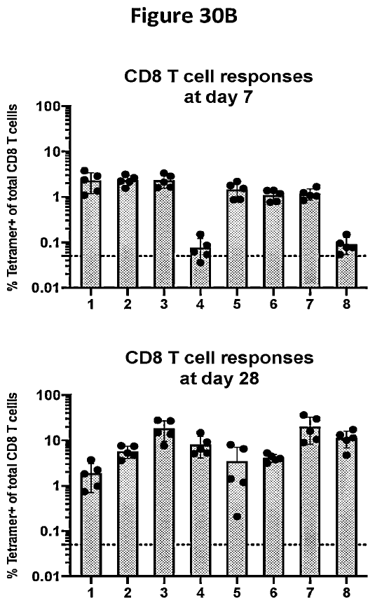

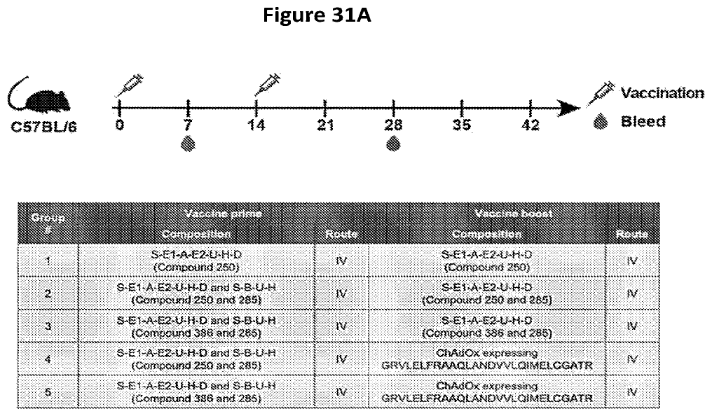

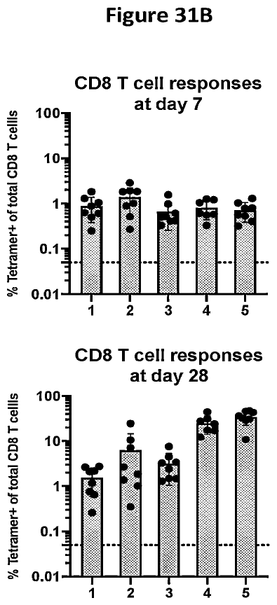

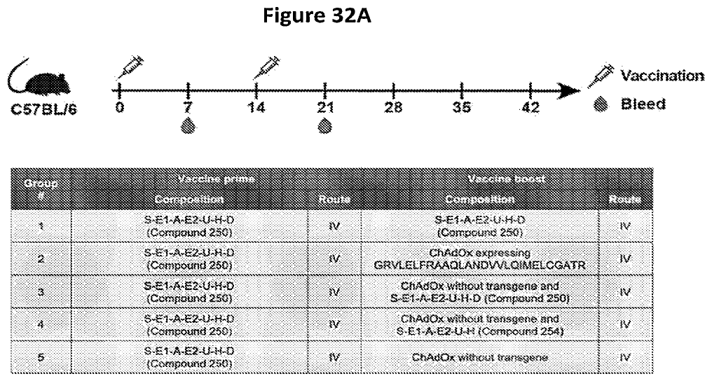

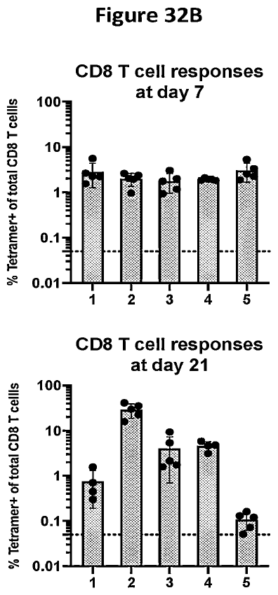

A striking finding was that peptide antigen conjugates comprising non-natural peptide antigens, wherein Met and Cys were replaced with nLeu and aBut, respectively, led to T cells that were cross-reactive for both the natural (native) peptide antigen and the non-natural sequence, and of a similar magnitude, phenotype and potency as compared T cells induced with the native peptide antigen (FIGS. 14-20). This was highly unexpected as it was unknown how substitution of amino acid residues within the T cell epitope might affect MHC binding, T cell priming and/or T cell reactivity. While nLeu and aBut were identified as suitable replacements for Met and Cys, respectively, an additional notable finding was that replacing methionine residues of peptide antigens with norvaline or leucine was deleterious to T cell responses (FIG. 16). Similarly, replacing Cys of naturally occurring peptide antigens with serine (Ser) resulted in non-natural antigens that led to significantly lower magnitude T cells induced against the native peptide antigen (FIG. 19).

TABLE 15
Peptide antigen conjugates comprising natural or non-naturally occurring peptide
antigen sequences

| | Name of | Peptide antigen conjugates | | | |
|---|---|---|---|---|---|
| Cmpd | peptide | S-E1-A-E2-U-H-D, | MW | MW | DH |
| # | antigen | where H = DBCO-Ahx-2B3W2 | Theor. | found | (nm) |
| 257 | Trp1 | KKKKKKKKKK-VR- | 7206.7 | Conf. | 18.0 |
| | | VTNTEMFVTAPDNLGYMYEVQWPGQE-SPVZ- | | | |
| | | X(H) (SEQ ID NO: 434) | | | |
| 258 | nor Trp1 | KKKKKKKKKK-VR- | 7189.1 | Conf. | 18.2 |
| | | VTNTEMFVTAPDNLGYnYEVQWPGQE-SPVZ- | | | |
| | | X(H) (SEQ ID NO: 435) | | | |
| 259 | Adpgk | KKKKKKKKK-VR- | 7168.9 | Conf. | 18.4 |
| | | GIPVHLELASMTNMELMSSIVHQQVFPT-SPVZ- | | | |
| | | X(H) (SEQ ID NO: 436) | | | |
| 260 | nor Adpgk | KKKKKKKKK-VR- | 7115.2 | Conf. | 19.7 |
| | | GIPVHLELASnTNnELnSSIVHQQVFPT-SPVZ- | | | |
| | | X(H) (SEQ ID NO: 437) | | | |
| 261 | norvaline | KKKKKKKKK-VR- | 7073.1 | Conf. | 28.2 |
| | Adpgk | GIPVHLELASvTNvELvSSIVHQQVFPT-SPVZ- | | | |
| | | X(H) (SEQ ID NO: 438) | | | |
| 262 | leucine Adpgk | KKKKKKKKK-VR- | 7115.2 | Conf. | 18.4 |
| | | GIPVHLELASLTNLELLSSIVHQQVFPT-SPVZ- | | | |
| | | X(H) (SEQ ID NO: 439) | | | |
| 263 | GP33 | KKKKKKK-VR-VITGIKAVYNFATCGIFAL- | 5803.7 | Conf. | |
| | | SPVZ-X(H) (SEQ ID NO: 440) | | | |
| 264 | aBut GP33 | KKKKKKK-VR-VITGIKAVYNFATBGIFAL- | 5785.7 | Conf. | |
| | | SPVZ-X(H) (SEQ ID NO: 441) | | | |
| 265 | Ser GP33 | KKKKKKK-VR-VITGIKAVYNFATSGIFAL-SPVZ- | 5787.6 | Conf. | |
| | | X(H) (SEQ ID NO: 442) | | | |
| 266 | M27 | KKKKKK-VR- | 6809.6 | Conf. | |
| | | REGVELCPGNKYEMRRHGTTHSLVIHD-SPVZ- | | | |
| | | X(H) (SEQ ID NO: 443) | | | |
| 267 | aBut M27 | KKKKKK-VR- | 6791.6 | Conf. | |
| | | REGVELBPGNKYEMRRHGTTHSLVIHD-SPVZ- | | | |
| | | X(H) (SEQ ID NO: 444) | | | |
| 268 | E7 | KKKKKKK-VR-GQAEPDRAHYNIVTFCCKCD- | 6072.8 | Conf. | |
| | | SPVZ-X(H) (SEQ ID NO: 445) | | | |
| 269 | aBut E7 | KKKKKKK-VR-GQAEPDRAHYNIVTFBBKBD- | 6018.8 | Conf. | |
| | | SPVZ-X(H) (SEQ ID NO: 446) | | | |

Single letter abbreviations are used for amino acid sequences in the above table; X = azidolysine, Z = citrulline; n = norleucine; v = norvaline; and B = alpha-aminobutyric acid. Peptide-based starting materials bearing a C-terminal azidolysine (X) were manufactured via solid-phase peptide synthesis by Genscript (Piscataway, NJ) and then reacted with a hydrophobic block fragment, Compound 136 (DBCO-Ahx-2B3W2), bearing DBCO to generate peptide antigen conjugates of Formula S-E1-A-E2-U-H(D). The reactions were monitored by HPLC and products were characterized by LC-MS to confirm that the molecular weight found conformed (“Conf.”) to theoretical and by DLS to assess hydrodynamic diameter (DH).
Note:
the underlined letters indicate the non-naturally occurring (‘non-native’) amino acid; e.g., the amino acid norleucine (“n”) of compound 258 replaced the methionine of compound 257. Trp1 is a self-antigen and norTrp1 is Trp1 except with a single methionine contained with a known T cell epitope replaced with norleucine. Adpgk is a tumor neoantigen and nor Adpk, norvaline Adpgk and leucine Adpgk are Adpgk except with the three methionine residues contained within a known T cell epitope replaced with either norleucine, norvaline or leucine, respectively. GP33 is a viral antigen and aBut GP33 and Ser GP33 are GP33 except a cysteine residue contained within a known T cell epitope is replaced with either aBut or Ser, respectively. M27 is a tumor neoantigen and aBut M27 is M27 except a cysteine residue contained within a known T cell epitope is replaced with aBut. E7 is a viral antigen and aBut E7 is E7 except the three cysteine residues have been replaced with aBut.

### Example 5: Impact of Amphiphile Composition on pH-Responsiveness

For use as solubilizing groups of amphiphiles, carboxylic acids should be deprotonated at pH near physiologic pH, e.g., pH 7.4, to ensure that the amphiphile has net negative charge. However, the pKa of carboxylic acids can be influenced by their chemical environment as well as substituent groups. Therefore, several amphiphiles comprising solubilizing groups further comprising carboxylic acids were synthesized and their solubility over a range of pH from pH 7.4 to pH 6.5 was evaluated.

Compounds 270, 271 and 160 comprise a PEG-based dendron amplifier with a terminal functional group (FGt) consisting of carboxylic acid that is the solubilizing group (SG) or is linked to SG via the linker X5, wherein X5-SG is —NH—(CH₂)—COOH and —NH—(CH₂)₂—COOH, respectively. Structures and synthesis of Compounds 270, 271 and 160 are provided below.

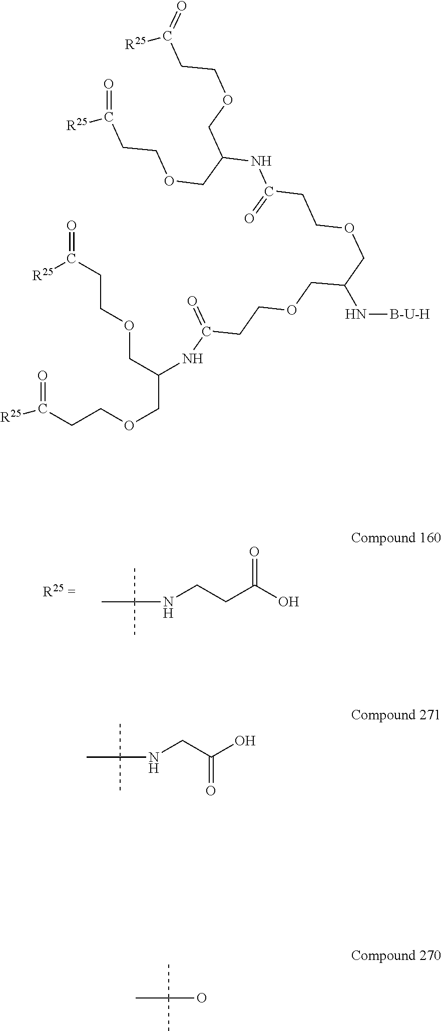

Compound 270, referred to (COOH)₄—PEG₂₄-(N₃-DBCO)-Ahx-2B₃W₂ or Tetra(COOH)-PEG₂₄-(N₃-DBCO)-Ahx-2B₃W₂ was synthesized by reacting Compound 145 with Compound 136 in a similar manner as described for Compound 154. MS (ESI) calculated for C₁₉₄H₂₈₃N₃₁O₅₂ m/z 3879.1, found 971.2 (M/4+H)⁺.

Compound 271, referred to (COOH-methyl)₄-PEG₂₄-(N₃-DBCO)-Ahx-2B₃W₂, Tetra(COOH-methyl)-PEG₂₄-(N₃-DBCO)-Ahx-2B₃W₂ or Tetra(Gly)-PEG₂₄-(N₃-DBCO)-Ahx-2B₃W₂ was synthesized in two steps. First, (COOH-methyl)₄-PEG₂₄-N₃ was synthesized by reacting Compound 146 with glycine in a similar manner as described for Compound 149. (COOH-methyl)₄-PEG₂₄-N₃ was then reacted with Compound 136 in a similar manner as described for Compound 154 to yield Compound 271. C₂₀₂H₂₉₅N₃₅O₅₆ m/z 4107.1, found 1370.4 (M/3+H)⁺.

Compounds 272, 273 and 274 comprise a peptide-based, i.e., lysine-based, dendron amplifier with a terminal functional group (FGt) consisting of an amine that is linked to SG via the linker X5, wherein X5-SG is —C(O)—(CH₂)₃—COOH for Compound 272 and X5-SG is —NH—(CH₂)₂—COOH for Compounds 273 and 274. Structures and synthesis of Compounds 272, 273 and 274 are provided below.

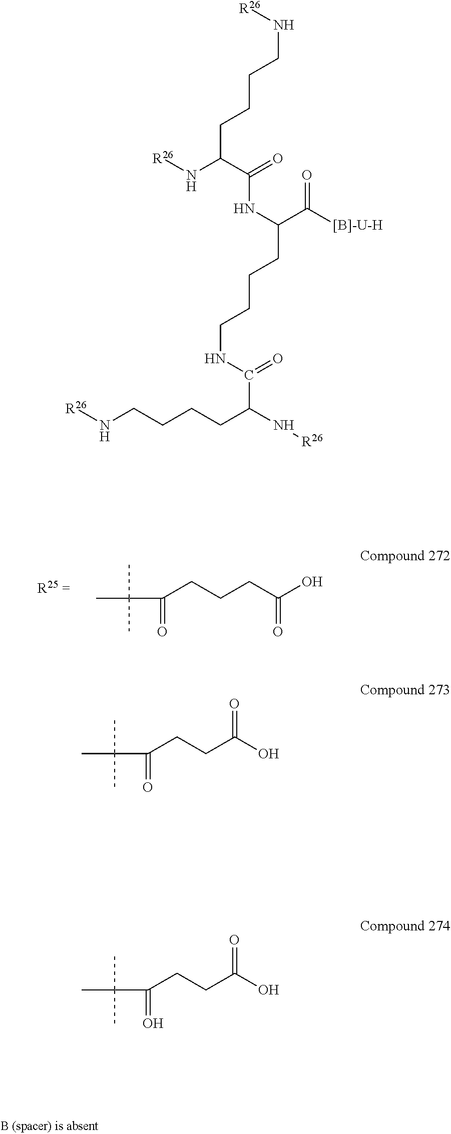

Compound 272, referred to as {Glutaric acid}4K2K{PEG24}{Lys(N3-DBCO)}-Ahx-2B₃W₂ was synthesized by reacting the peptide-based dendron comprising a C-terminal Lys(N3), {Glutaric acid}4K2K{PEG24}{Lys(N3)}, with Compound 136 in a similar manner as was described for Compound 154. MS (ESI) calculated for C₂₁₁H₃₁₁N₃₇O₅₂ m/z 4195.3, found 1050.2 (M/4+H)⁺.

Compound 273, referred to as {Succinic acid}4K2KK{PEG24-N3-DBCO)}-Ahx-2B₃W₂ was synthesized in two steps. First, the epsilon amine of the C-terminal lysine of the peptide-based dendron {Succinic acid}4K2KK was reacted with NHS-PEG24-N₃ to yield {Succinic acid}4K2KK{PEG24-N3}, which was then reacted with Compound 136 in a similar manner as was described for Compound 154 to yield Compound 273. MS (ESI) calculated for C₂₀₇H₃₀₃N₃₇O₅₂ m/z 4139.2, found 1036.3 (M/4+H)⁺.

Compound 274, referred to as {Succinic acid}4K2K{Lys(N3-DBCO)}-Ahx-2B₃W₂ was synthesized by reacting the peptide-based dendron comprising a C-terminal Lys(N3), {Succinic acid}4K2K {Lys(N3)}, with Compound 136 in a similar manner as was described for Compound 154. MS (ESI) calculated for C₁₅₆H₂₀₂N₃₆O₂₇ m/z 3011.6, found 1005.3 (M/3+H)+.

The pH-responsive properties of the 6 different compositions (Compounds 160, 270, 271, 272, 273 and 274) of amphiphiles further comprising carboxylic acid groups was evaluated using turbidity measurements. In brief, each amphiphile was suspended in 1×PBS buffer at either pH 7.4, 7.0 or pH 6.5 at a final concentration of 0.1 mM and turbidity (OD at 490 nm) was assessed using a UV-Vis spectrophotometer (FIG. 21).

Except for Compound 271, which exhibited some aggregation at pH 7.4, all the other amphiphiles comprising carboxylic acids formed nanoparticle micelles with stable particle size at pH 7.4 (FIG. 21B). Notably, Compound 270 formed aggregates at pH 7.0 and pH 6.5, indicating pH-responsiveness near physiologic pH, between about pH 7.4 and 7.0, whereas Compounds 160, 272, 273 and 274 exhibited stable particle sizes down to at least pH 7.0.

### Example 6: Impact of Peptide Antigen Conjugate (PAC) Net Charge and Amphiphile

Vaccines comprising nanoparticles further comprising one or more peptide antigen conjugates with average net charge of the peptide antigen conjugates of greater than or equal to +8 were found to cause red blood cell (RBC) lysis in a dose-dependent manner. It was hypothesized that dose-dependent hemolysis of vaccines comprising positively charged peptide antigen conjugates could be mitigated by either or both reducing the average net charge of the peptide antigen conjugates and or using neutral or negatively charged amphiphilic carriers as shielding groups. Moreover, as certain amphiphiles can promote nanoparticle stability, it was further hypothesized that use of certain amphiphiles could reduce the average peptide antigen conjugate net charge needed to stabilize particles comprising amphiphiles and one or more peptide antigen conjugates.

Thus, to evaluate how the interplay between peptide antigen conjugate net charge and amphiphile composition impacts the size, stability and RBC lysing potential of vaccines comprising one or more peptide antigen conjugates and an amphiphile, vaccines with varying amphiphile composition and peptide antigen conjugate net charge were evaluated for RBC lysing potential, zeta potential and particle size stability and are summarized in Table 16 and FIG. 22.

TABLE 16
vaccine compositions included in FIG. 22 studies.

| | | | | Ratio of |
|---|---|---|---|---|
| Group | | PAC net | Amphi- | PAC to |
| # | Peptide antigen conjugate(s) | charge | phile | Amph. |
| 1 | 253, 255, 256, 280, 281 | +8 | None | N.A. |
| 2 | 253, 255, 256, 280, 281 | +8 | 285 | 1:1 |
| 3 | 253, 255, 256, 280, 281 | +8 | 286 | 1:1 |
| 4 | 253, 255, 256, 280, 281 | +8 | 287 | 1:1 |
| 5 | 275, 276, 277, 278, 279 | +3 | 285 | 1:1 |
| 6 | 275, 276, 277, 278, 279 | +3 | 286 | 1:1 |
| 7 | 275, 276, 277, 278, 279 | +3 | 287 | 1:1 |
| 8 | 222, 221, 220, 275, 276 | Native | 285 | 1:1 |
| | | antigens, +1 | | |
| 9 | 222, 221, 220, 275, 276 | Native | 286 | 1:1 |
| | | antigens, +1 | | |
| 10 | 222, 221, 220, 275, 276 | Native | 287 | 1:1 |
| | | antigens, +1 | | |
| 11 | None | N.A. | 285 | N.A. |
| 12 | None | N.A. | 286 | N.A. |
| 13 | None | N.A. | 287 | N.A. |

Peptide antigen conjugates used to prepare the formulations described in Table 16 are summarized in Table 17 or elsewhere.

TABLE 17
Additional peptide antigen conjugates of formula [S]-[E1]-A-[E2]-U-H.

| Cmpd | [S]-[E1]-A-[E2]-U1 | U2-H | Theo | Found |
|---|---|---|---|---|
| # | Product | | MW | MW |
| 275 | nEVGWYRSPFSRVVHLYRNGKSPVZX (SEQ ID | Compound 62 | 4524.9 | 1131.8 |
| | NO: 447) | | | (M/4 + H)⁺ |
| | nEVGWYRSPFSRVVHLYRNGKSPVZX(DBCO-Ahx-W5) | | | |
| 276 | RLERVBHBLGKWLGHPDKFVGITYASPVZX (SEQ ID | Compound 62 | 4806.3 | 1202.2 |
| | NO: 448) | | | (M/4 + H)⁺ |
| | RLERVBHBLGKWLGHPDKFVGITYASPVZX(DBCO-Ahx-W5) | | | |
| 277 | KKKVRGIPVHLELASMTNMELMSSIVHQQVFPTX | Compound 62 | 5252.9 | 1313.6 |
| | (SEQ ID NO: 406) | | | (M/4 + H)⁺ |
| | KKKVRGIPVHLELASMTNMELMSSIVHQQVFPTX(DBCO-Ahx-W5) | | | |
| 278 | KKVRKARDETAALLNSAVLGAAPLFVPPADX (SEQ ID | Compound 62 | 4622.1 | 1155.8 |
| | NO: 449) | | | (M/4)⁺ |
| | KKVRKARDETAALLNSAVLGAAPLFVPPADX(DBCO-Ahx-W5) | | | |
| 279 | KKKKVRDFTGSNGDPSSPYSLHYLSPTGVNEYX (SEQ | Compound 62 | 5075.3 | 1269.2 |
| | ID NO: 407) | | | (M/4 + H)⁺ |
| | KKKKVRDFTGSNGDPSSPYSLHYLSPTGVNEYX(DBCO-Ahx-W5) | | | |
| 280 | KKKVRnEVGWYRSPFSRVVHLYRNGKSPVZX (SEQ | Compound 62 | 5146.7 | 1287.2 |
| | ID NO: 450) | | | (M/4 + H)⁺ |
| | KKKVRnEVGWYRSPFSRVVHLYRNGKSPVZX(DBCO-Ahx-W5) | | | |
| 281 | KKKKKVRLERVBHBLGKWLGHPDKFVGITYASPVZX | Compound 62 | 5546.3 | 1387.0 |
| | (SEQ ID NO: 451) | | | (M/4 + H)⁺ |
| | KKKKKVRLERVBHBLGKWLGHPDKFVGITYASPVZX(DBCO-Ahx-W5) | | | |
| 282 | SLKISQAVHAAHAEINEAGREVVGSPVZX (SEQ ID | Compound 62 | 4524.9 | 1140.2 |
| | NO: 452) | | | (M/4 + H)⁺ |
| | ESLKISQAVHAAHAEINEAGREVVGSPVZX(DBCO-Ahx-W5) | | | |
| 283 | KKKVRESLKISQAVHAAHAEINEAGREVVGSPVZX | Compound 62 | 5198.6 | |
| | (SEQ ID NO: 453) | | | |
| | KKKVRESLKISQAVHAAHAEINEAGREVVGSPVZX(DBCO-Ahx-W5) | | | |
| 284 | KKKKKKKKVRESLKISQAVHAAHAEINEAGREVVGS | Compound 62 | 5839.5 | 1460.4 |
| | PVZX (SEQ ID NO: 454) | | | (M/4 + H)⁺ |

KKKKKKKKVRESLKISQAVHAAHAEINEAGREVVGSPVZX(DBCO-
Ahx-W5)

Single letter abbreviations are used for amino acid sequences in the above table; X = azidolysine, Z = citrulline; n = norleucine; and B = alpha-aminobutyric acid. Peptide-based starting materials bearing a C-terminal azidolysine (X) were manufactured via solid-phase peptide synthesis by Genscript (Piscataway, NJ) and then reacted with a hydrophobic block fragment, Compound 62 (DBCO- Ahx-W5), bearing DBCO to generate peptide antigen conjugates of Formula [S]-[E1]-A-[E2]-U-H. The reactions were monitored by HPLC and products were characterized by LC-MS to confirm that the molecular weight found conformed to theoretical.

Amphiphiles used to prepare the formulations described in Table 16 are summarized below or elsewhere.

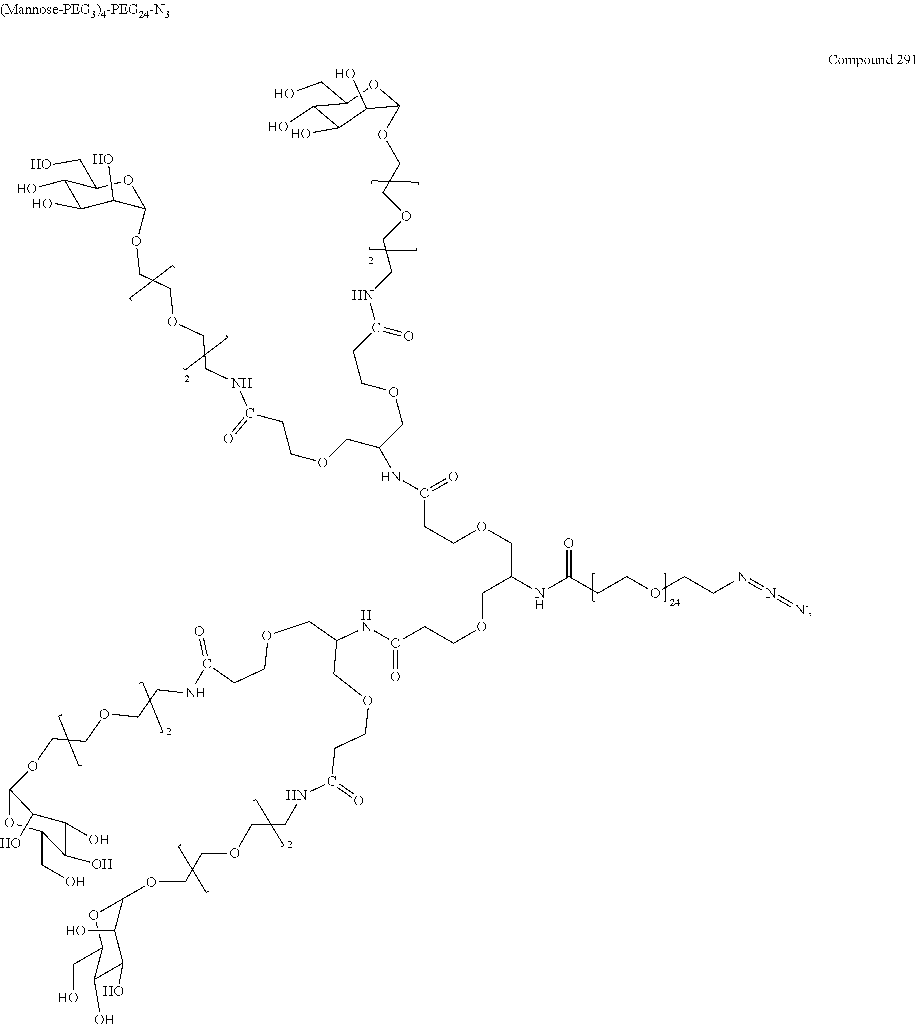

Compound 291, referred to as (Mannose-PEG₃)₄-PEG₂₄-N₃ or Tetra(Mannose-PEG₃)-PEG₂₄-N₃ was synthesized following the same procedure as Compound 147, except a-Mannose-PEG₃-amine (CarboSynthUSA (San Diego, CA)) was used instead of N-boc-ethylenediamine and DMSO was used as the solvent. Compound 291 was purified on a preparatory HPLC system using a gradient of 15-35% acetonitrile/H₂O (0.05% TFA) over 12 minutes on an Agilent Prep-C18 column, 50×100 mm, 5 μm. The product eluted at ˜7.3 minutes and the resulting fractions were collected, frozen and then lyophilized to obtain a 49.5% yield of a spectroscopically pure (97.2% AUC at 220 nm) white solid. MS (ESI) Calculated for C₁₂₆H₂₃₈N₁₀O₆₉ m/z 2995.5, found 999.9 (M/3)⁺.

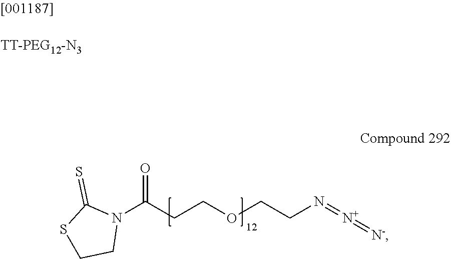

Compound 292, referred to as TT-PEG₁₂-N₃ was synthesized by reacting 50 mg of N₃-PEG₁₂-COOH (0.08 mmol, 1 eq) with 33.0 mg of HATU (0.09 mmol, 1.4 eq) in 330 μL of DCM. To the mixture, 43.3 uL of triethylamine (0.31 mmol, 4 eq) was added. The mixture was stirred for 5 minutes and 12.6 mg of thizoline-2-thio (TT) (0.11 mmol, 1.4 eq) was added. The reaction mixture stirred at room temperature for 2 hours until HPLC indicated the reaction was complete. The product was purified by flash chromatography on a 10 g Biotage Safar Silica HC column over a 2-step gradient: 0% methanol in DCM over 3 column volumes (CVs), followed by 0-7% methanol in DCM over 20 CVs. The product eluted at ˜5% methanol and the resulting fractions were collected and the solvent removed to obtain 24 mg (41.0% yield) of a spectroscopically pure (92.0% AUC at 220 nm) yellow oil. MS (ESI) Calculated for C₃₀H₅₆N₄O₁₃S₂ m/z 744.3, found 745.3 (M+H)⁺.

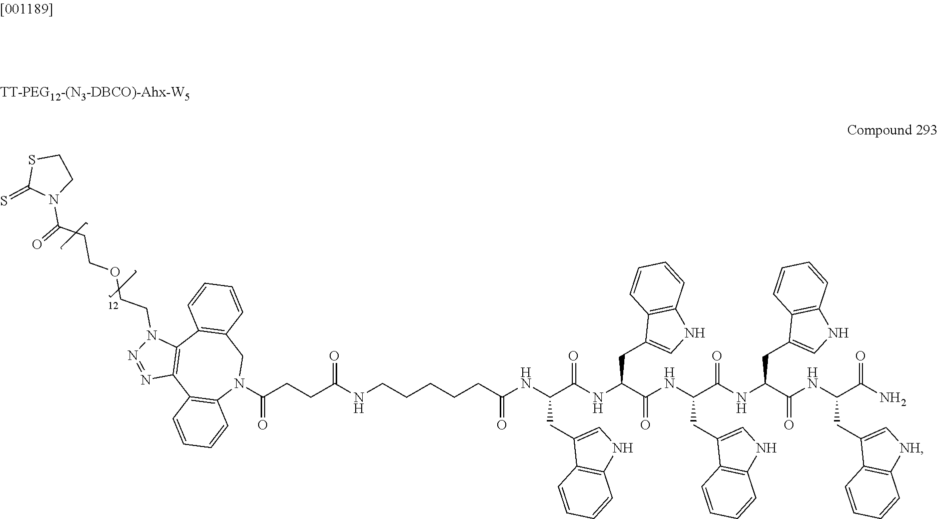

Compound 293, referred to as TT-PEG₁₂-(N₃-DBCO)-Ahx-W₅ was synthesized by reacting 14.2 mg of Compound 292 (0.02 mmol, 1 eq) with 28.4 mg of Compound 62 (0.02 mmol, 1.1 eq) in 400 μL of anhydrous DMSO. The reaction was mixed at room temperature for 16 hours, until HPLC indicated the reaction was complete. The reaction was not purified and resulted in an 83% pure (AUC at 220 nm) product. MS (ESI) Calculated for C₁₁₀H₁₃₃17O₂₁S₂ m/z 2091.9, found 1048.4 (M/2+H)⁺.

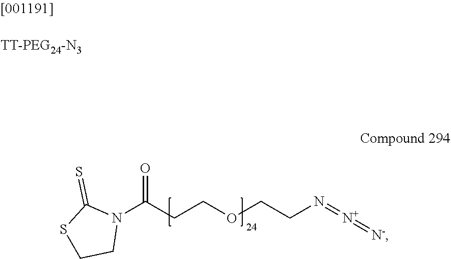

Compound 294, referred to as TT-PEG₂₄-N₃ was synthesized by reacting 2.55 g of N₃-PEG₂₄-COOH (2.2 mmol, 1 eq) with 0.92 g of HATU (2.4 mmol, 1.1 eq) in 24 mL of DCM. To the mixture, 1.2 mL of triethylamine (8.7 mmol, 4 eq) was added. The mixture was stirred for 5 minutes and 0.29 g of thizoline-2-thio (TT) (2.5 mmol, 1.1 eq) was added. The reaction mixture was stirred at room temperature for 2 hours until HPLC indicated the reaction was complete. The reaction mixture was diluted with 200 mL of dichloromethane (DCM) and then washed with 2×200 mL 0.1 M HCl and the 1×200 mL DI H₂O. The organic layer was dried with Na₂SO₄ and then removed under vacuum, resulting in a yellow oil. The product was then purified by flash chromatography on a 100 g Biotage Safar Silica HC column over a 2-step gradient: 0% methanol in DCM over 3 column volumes (CVs), followed by 0-8% methanol in DCM over 20 CVs. The product eluted at ˜5% methanol and the resulting fractions were collected and the solvent removed to obtain 1.8 g (62.5% yield) of an 84% pure (AUC at 220 nm) yellow oil. MS (ESI) Calculated for C₅₄H₁₀₄N₄O₂₈S₂ m/z 1272.6, found 1273.6 (M+H)⁺.

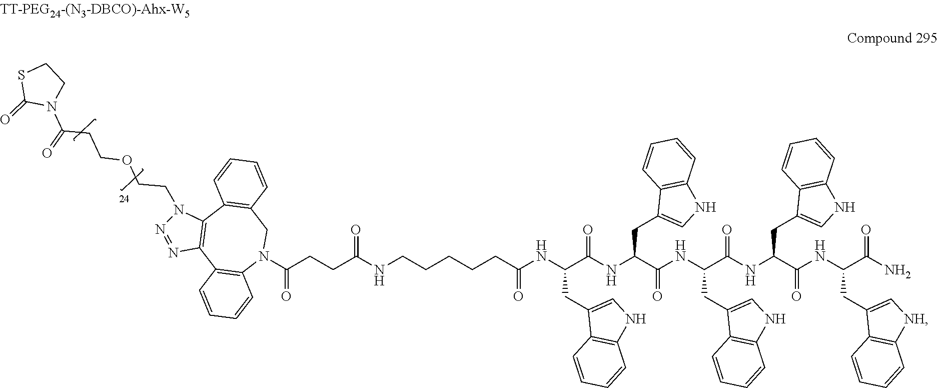

Compound 295, referred to as TT-PEG₂₄-(N₃-DBCO)-Ahx-W₅ was synthesized by reacting 12.7 mg of Compound 294 (0.01 mmol, 1 eq) with 14.8 mg of Compound 62 (0.01 mmol, 1.1 eq) in 500 μL of DMSO. The reaction was mixed at room temperature for 16 hours, until HPLC indicated the reaction was complete. The reaction was not purified and resulted in an 84% pure (AUC at 220 nm) product. MS (ESI) Calculated for C₁₃₄H₁₈₁N₁₇O₃₃S₂ m/z 2622.1, found 1311.8 (M/2+H)⁺.

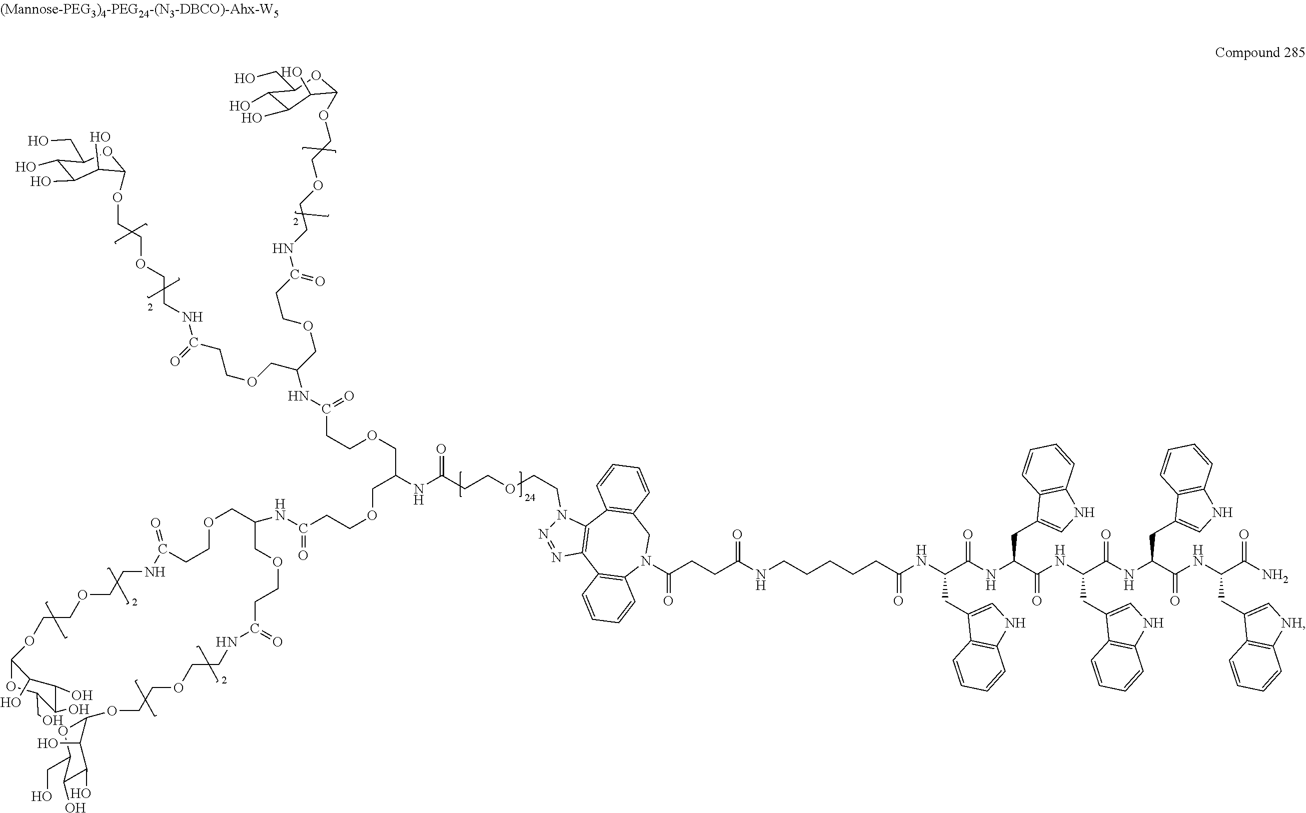

Compound 285, referred to (Mannose-PEG₃)₄-PEG₂₄-(N₃-DBCO)-Ahx-W₅ or Tetra(Mannose-PEG₃)-PEG₂₄-(N₃-DBCO)-Ahx-W₅ was synthesized by reacting 1 equivalent of Compound 291 with 1 equivalent of Compound 62 in anhydrous DMSO for 16 hours at room temperature. HPLC was monitored to evaluate reaction progress and indicated complete conversion of Compound 291 to Compound 285, resulting in a spectroscopically pure (94.0% AUC at 220 nm) colorless solution. MS (ESI) calculated for C₂₀₆H₃₁₅N₂₃O₇₇ m/z 4343.21 found 1449.0 (M/3+H)⁺.

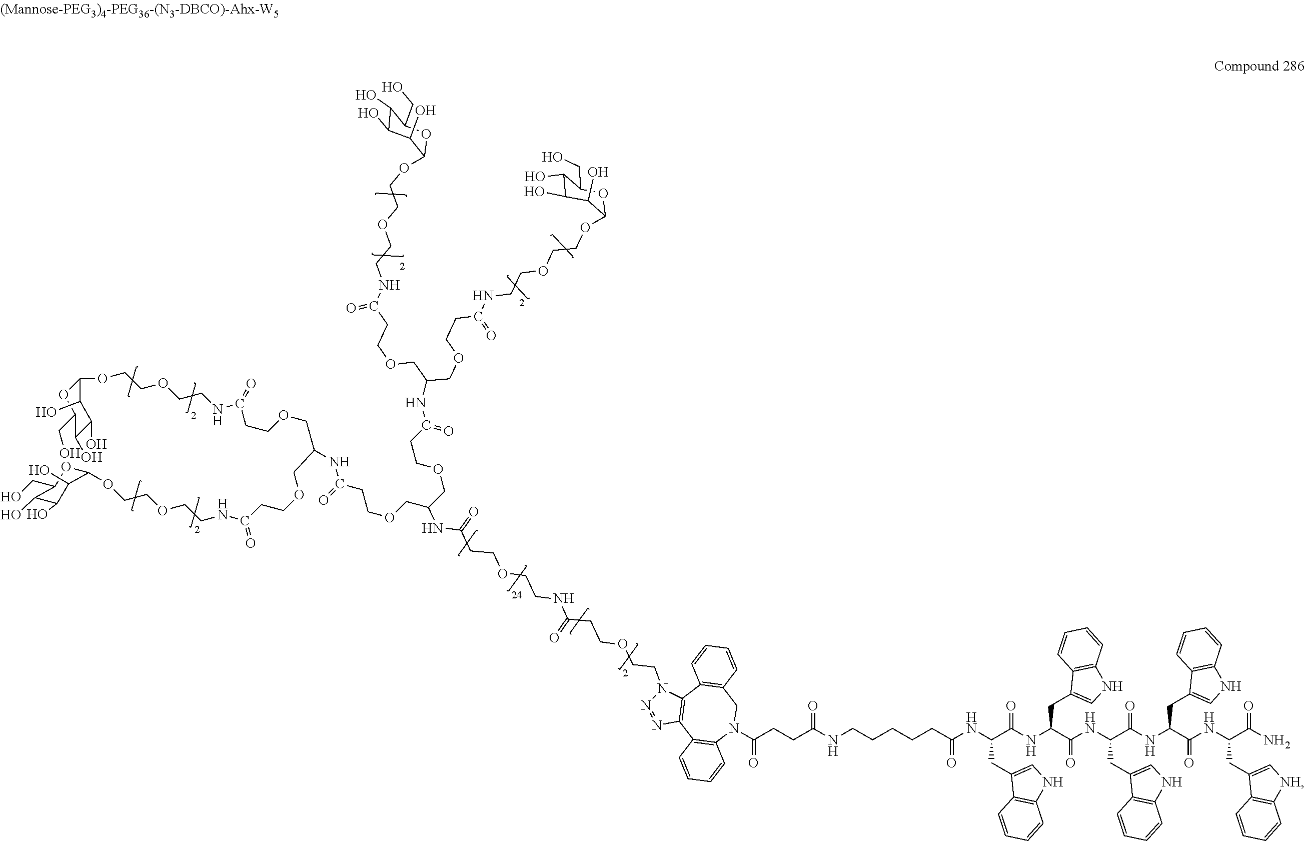

Compound 286, referred to as (Mannose-PEG₃)₄-PEG₂₄-PEG₁₂-(N₃-DBCO)-Ahx-W₅, (Mannose-PEG₃)₄-PEG₃₆-Ahx-W₅ or Tetra(Mannose-PEG₃)-PEG₃₆-Ahx-W₅, was synthesized by first synthesizing (Mannose-PEG₃)₄-PEG₂₄-NH₂ by reacting 45 mg Compound 291 (15 umol, 1 eq) with 43 mg tris(2-carboxyethyl)phosphine hydrochloride (TCEP) (150 umol, 10 eq) in 1 mL anhydrous DMSO. The reaction was mixed for 16 hours at room temperature, when HPLC indicated that all of Compound 291 was converted to (Mannose-PEG₃)-PEG₂₄-NH₂. To the reaction mixture 28.9 mg of Compound 293 (13.8 umol, 1 eq) and 40 uL triethylamine (TEA) (287 umol, 20 eq) was added. The reaction was stirred for 1 hour at room temperature, when HPLC indicated the reaction was complete. Compound 286 was purified on a preparatory HPLC system using a gradient of 32-52% acetonitrile/H₂O (0.05% TFA) over 12 minutes on an Agilent Prep-C18 column, 50×100 mm, 5 μm. The product eluted at ˜6 minutes and the resulting fractions were collected, frozen and then lyophilized to obtain a 47.9% yield of a spectroscopically pure (96.2% AUC at 220 nm) colorless oil. MS (ESI) Calculated for C₂₃₃H₃₆₈N₂₄O₉₀ m/z 4942.5, found 1237.2 (M/4+H)⁺.

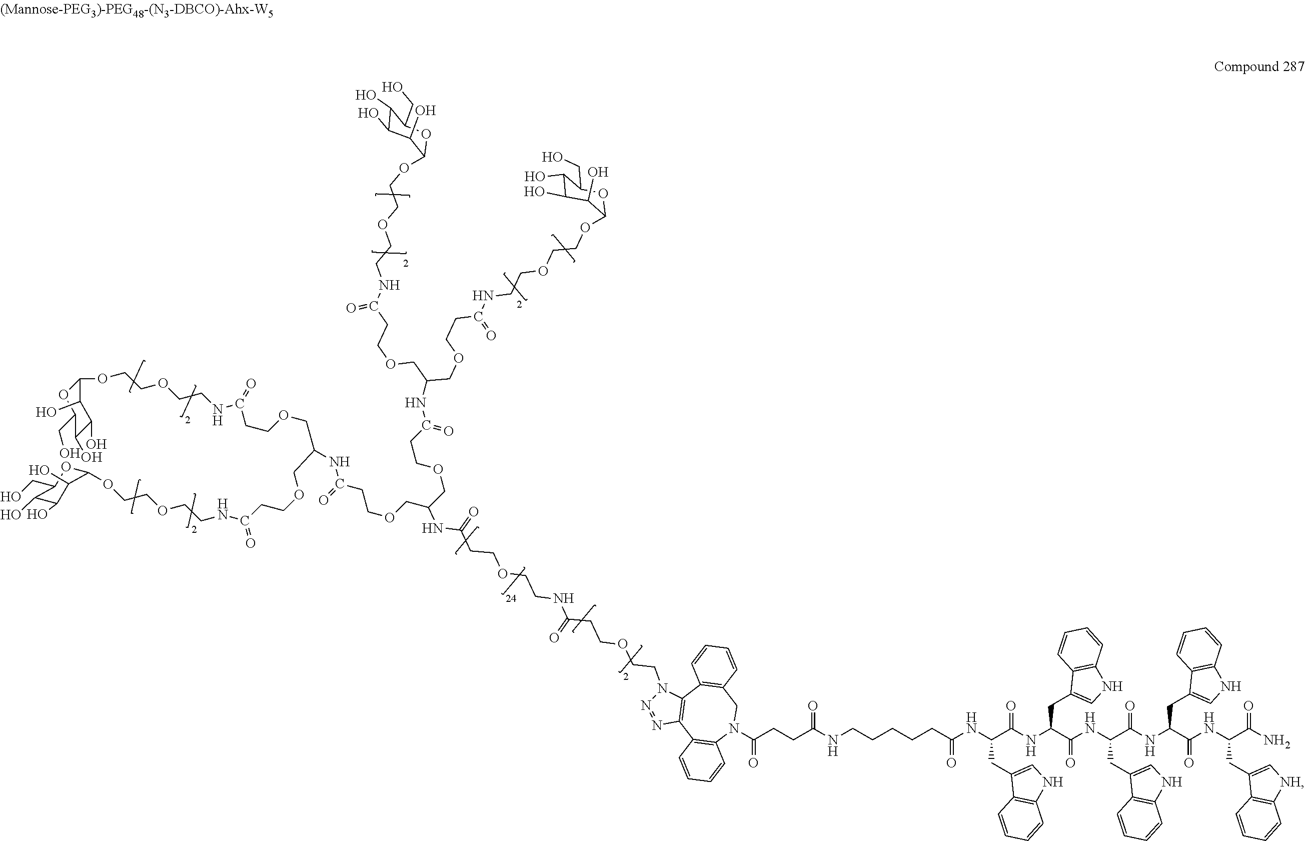

Compound 287, referred to as (Mannose-PEG₃)₄-PEG₂₄-PEG₂₄-Ahx-W₅, (Mannose-PEG₃)₄-PEG₄₈-Ahx-W₅, Tetra(Mannose-PEG₃)-PEG₄₈-Ahx-W₅ was synthesized following the same procedure at Compound 286, except that once Compound 291 was fully converted to (Mannose-PEG₃)-PEG₂₄-NH₂, Compound 295 was added instead of Compound 293. Compound 287 was purified on a preparatory HPLC system using a gradient of 32-52% acetonitrile/H₂O (0.05% TFA) over 12 minutes on an Agilent Prep-C18 column, 30×100 mm, 5 μm. The product eluted at ˜7 minutes and the resulting fractions were collected, frozen and then lyophilized to obtain a 40.9% yield of a spectroscopically pure (98.2% AUC at 220 nm) colorless oil. MS (ESI) Calculated for C₂₅₇H₄₁₆N₂₄O₁₀₂ m/z 5470.8, found 1095.9 (M/5+H)⁺.

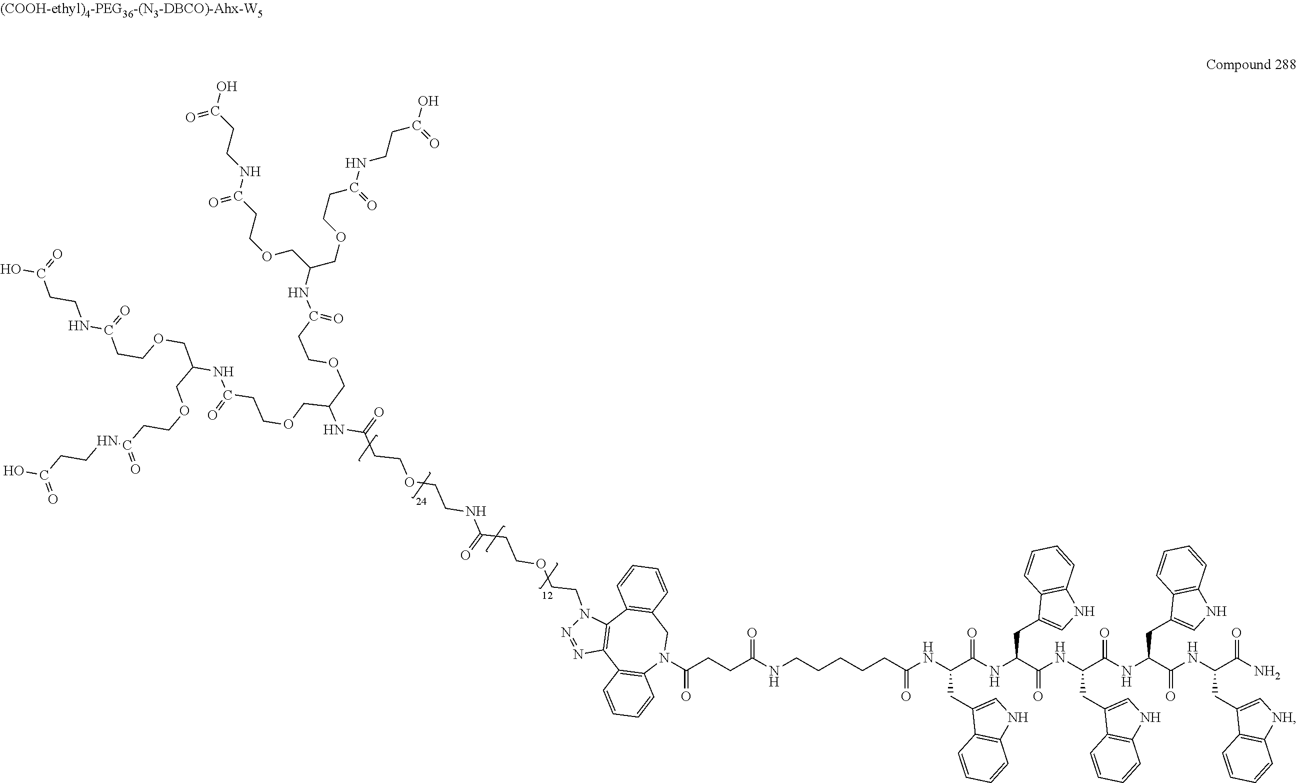

Compound 288, referred to as referred to as (COOH-ethyl)₄-PEG₂₄-PEG₁₂-(N₃-DBCO)-Ahx-W₅, (COOH-ethyl)₄-PEG₃₆-(N₃-DBCO)-Ahx-W₅ or Tetra(COOH-ethyl)-PEG₃₆-Ahx-W₅, was synthesized following the same procedure as Compound 286, except Compound 149 was used in place of Compound 291. Compound 288 was purified on a preparatory HPLC system using a gradient of 34-54% acetonitrile/H₂O (0.05% TFA) over 12 minutes on an Agilent Prep-C18 column, 30×100 mm, 5 μm. The product eluted at ˜7 minutes and the resulting fractions were collected, frozen and then lyophilized to obtain a 62.3% yield of a spectroscopically pure (94.6% AUC at 220 nm) colorless oil. MS (ESI) Calculated for C₁₉₇H₂₉₆N₂₄O₆₆ m/z 4054.1, found 1352.6 (M/3+H)⁺.

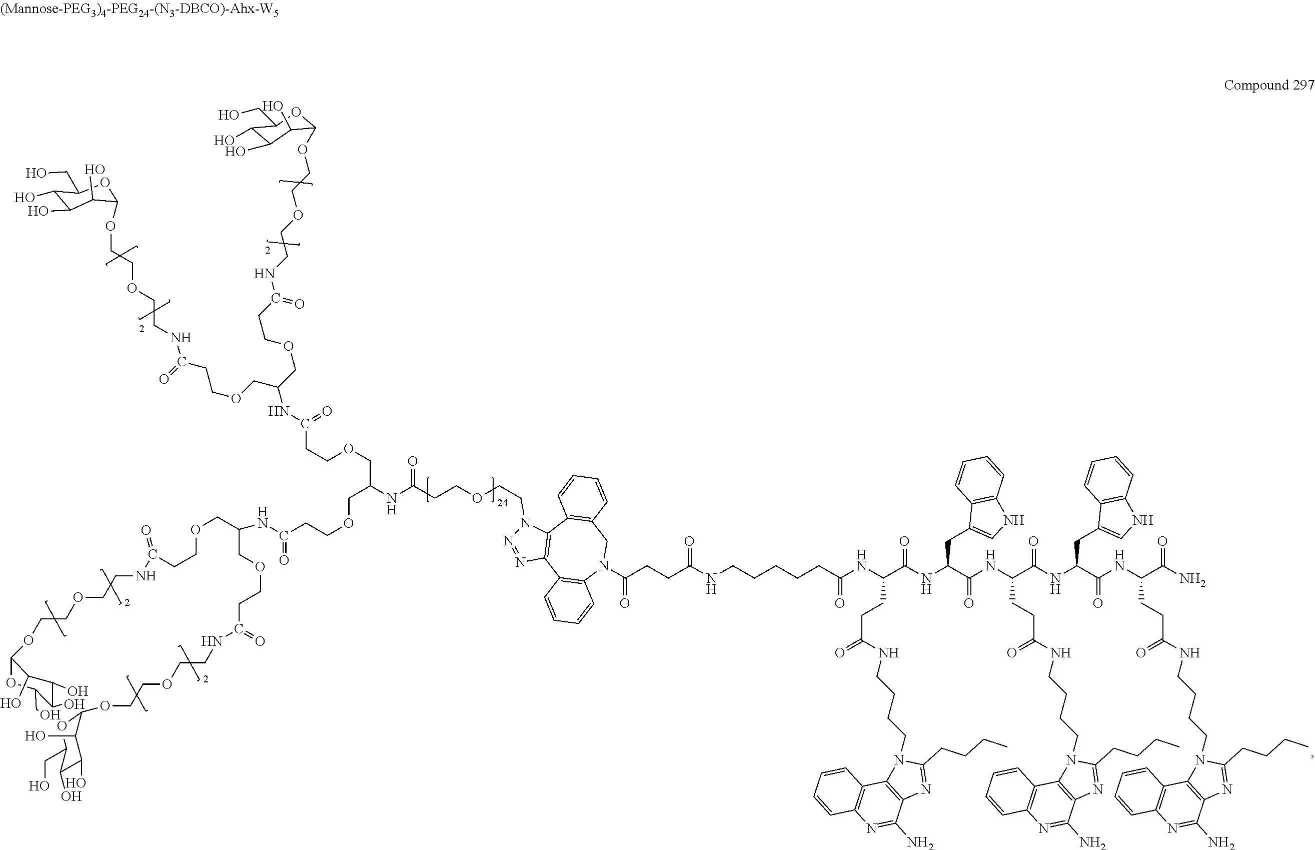

Compound 297, referred to (Mannose-PEG₃)₄-PEG₂₄-(N₃-DBCO)-Ahx-2B₃W₂ or Tetra(Mannose-PEG₃)-PEG₂₄-(N₃-DBCO)-Ahx-2B₃W₂ was synthesized by reacting 1 equivalent of Compound 291 with 1 equivalent of Compound 136 in anhydrous DMSO for 16 hours at room temperature. HPLC was monitored to evaluate reaction progress and indicated complete conversion of Compound 291 to Compound 297, resulting in a spectroscopically pure (95.0% AUC at 220 nm) colorless solution. MS (ESI) calculated for C₂₄₂H₃₇₅N₂₅O₈₀ m/z 5051.6 found 1264.6 (M/4+H)⁺.

To assess how the vaccine composition impacts red blood cell (RBC) lysing potential, the various vaccine compositions summarized in Table 16 were incubated at 0.1 mM final concentration with murine red blood cells at 8% v/v PBS for 3 hours at 37C and % tumor lysis was calculated based on 1% triton X and PBS buffer as 100% hemolysis and 0% hemolysis controls, respectively.

A striking finding was that the vaccines comprising a neutral amphiphile resulted in significantly lower RBC lysis as compared with vaccines without a neutral amphiphile and that the extent of hemolysis was proportional to the average net charge of the peptide antigen conjugates included in the vaccine (FIG. 22A). Accordingly, vaccines comprising peptide antigen conjugates with average net charge of +8 had zeta potential >15 mV and caused >10% of RBCs to lyse, whereas vaccines comprising peptide antigen conjugates with average net charge of +8 and a neutral amphiphile still had positive zeta potential (i.e., ˜20 mV) but induced significantly less RBC lysis (<5%). Moreover, vaccines comprising peptide antigen conjugates with either +3 or +1 average net charge and further comprising a neutral amphiphile exhibited still lower zeta potential and hemolysis than vaccines comprising peptide antigen conjugates with +8 average net charge and a neutral amphiphile.

While decreasing peptide antigen conjugate charge was found to lead to decreased RBC lysis, vaccine particle size stability decreased with decreasing charge (FIG. 22B). Notably, vaccines comprising peptide antigen conjugates with average net charge of +8 with or without an amphiphile, and vaccines comprising peptide antigen conjugates with average net charge of +3 with an amphiphile, formed nanoparticles that were stable for at least 20 hours at room temperature, whereas vaccines comprising peptide antigen conjugates with near neutral charge, i.e, +1 average net charged, exhibited a propensity to aggregate after 20 hours at room temperature. These findings were substantiated with the evaluation of additional vaccine compositions. Accordingly, vaccine compositions comprising nanoparticles with different amphiphiles (Compound 152, 153, 156 or 162) and peptide antigen conjugates with net charge varying from −2 to +5 (Compound 201, 202, 203, 204, 205 or 206) at a 1:1 molar ratio were evaluated for particle size stability. The results show that vaccines comprising amphiphiles and peptide antigen conjugates with average net charge between +3 and +5 result in nanoparticle micelles, whereas compositions with net charge less than +3 generally form aggregates (FIG. 23).

To further investigate how the interplay between peptide antigen conjugate net charge and amphiphile properties impacts the particle size stability of vaccine compositions comprising one or more peptide antigen conjugates and an amphiphile, vaccine compositions (Table 18) with varying amphiphile composition and peptide antigen conjugate net charge were evaluated for particle size stability over multiple freeze-thaw cycles and are summarized in FIG. 24. The results show that vaccines comprising peptide antigen conjugates with average net charge greater than or equal to +3 and a neutral amphiphile at a 1:1 ratio are stable over at least three freeze-thaw cycles, but that vaccines comprising peptide antigen conjugates with average net charge near neutral, e.g., -1, and a neutral amphiphile at a 1:1 ratio begin to exhibit aggregation after three freeze-thaw cycles (FIG. 24).

TABLE 18
vaccine compositions included in FIG. 24 studies.

| | | | | Ratio of |
|---|---|---|---|---|
| Group | Peptide antigen | | Amphi- | PAC to |
| # | conjugate(s) | PAC net charge | phile | Amph. |
| 1 | 253, 255, 256 | +8 | None | N.A. |
| 2 | 253, 255, 256 | +8 | 285 | 1:1 |
| 3 | 253, 255, 256 | +8 | 287 | 1:1 |
| 4 | 289, 290, 296 | +5 | None | N.A |
| 5 | 289, 290, 296 | +5 | 285 | 1:1 |
| 6 | 289, 290, 296 | +5 | 287 | 1:1 |
| 7 | 277, 278, 279 | +3 | 285 | 1:1 |
| 8 | 277, 278, 279 | +3 | 287 | 1:1 |
| 9 | 220, 221, 222 | Native antigens, −1 | 285 | 1:1 |
| 10 | 220, 221, 222 | Native antigens, −1 | 287 | 1:1 |
| 11 | None | N.A. | 285 | N.A. |
| 12 | None | N.A. | 287 | N.A. |

Peptide antigen conjugates used to prepare the formulations described in Table 18 are summarized in Table 19 or elsewhere.

TABLE 19
Additional peptide antigen conjugates of formula [S]-[E1]-A-[E2]-U-H.

| Cmpd | [S]-[E1]-A-[E2]-U1 | U2-H | Theo | Found |
|---|---|---|---|---|
| # | Product | | MW | MW |
| 289 | KKKKKVRGIPVHLELASMTNMELMSSIVHQQVFPTX | Compound 62 | 5509.3 | 1377.6 |
| | (SEQ ID NO: 408) | | | (M/4 + H)⁺ |
| | KKKKKVRGIPVHLELASMTNMELMSSIVHQQVFPTX(DBCO-Ahx-W5) | | | |
| 290 | KKKKVRKARDETAALLNSAVLGAAPLFVPPADX | Compound 62 | 4878.4 | 1220/4 |
| | (SEQ ID NO: 455) | | | (M/4 + H)⁺ |
| | KKKKVRKARDETAALLNSAVLGAAPLFVPPADX(DBCO-Ahx-W5) | | | |
| 296 | KKKKKKVRDFTGSNGDPSSPYSLHYLSPTGVNEYX | Compound 62 | 5331.7 | 1333.2 |
| | (SEQ ID NO: 409) | | | (M/4 + H)⁺ |

KKKKKKVRDFTGSNGDPSSPYSLHYLSPTGVNEYX(DBCO-Ahx-W5)

Single letter abbreviations are used for amino acid sequences in the above table; X = azidolysine, Z = citrulline; n = norleucine; and B = alpha-aminobutyric acid. Peptide-based starting materials bearing a C-terminal azidolysine (X) were manufactured via solid-phase peptide synthesis by Genscript (Piscataway, NJ) and then reacted with a hydrophobic block fragment, Compound 62 (DBCO-Ahx-W5), bearing DBCO to generate peptide antigen conjugates of Formula [S]-[E1]-A-[E2]-U-H. The reactions were monitored by HPLC and products were characterized by LC-MS to confirm that the molecular weight found conformed to theoretical.

### Example 7: Selection of Immunomodulatory Drug Molecules for Tolerance Vaccines

Most tolerance vaccines evaluated to-date comprise an antigen selected from autoantigens, allergens or allergens alone or additional include an mTOR inhibitor selected from Rapamycin or an aryl hydrocarbon receptor agonist. Immunostimulants have not been used in tolerance vaccines likely because the effects of immunistimulants, e.g., induction of APC activation, pro-inflammatory cytokine production and priming of helper T cells and/or cytotoxic T cells are counter to the objectives of a tolerance vaccine. However, use of antigen either alone or with an immunosuppressant result in a relatively low magnitude of regulatory T cells as such vaccine formulations lack immunological cues to drive robust T cell priming and expansion.

It was hypothesized by the inventors of the present disclosure that the magnitude of regulatory T cells induced with tolerance vaccines could be increased by combining certain immunostimulants with inhibitors of T cell differentiation as means to drive priming and expansion of T cells with the immunostimulant but blocking their differentiation and expansion with an inhibitor, thereby driving the primed and expanded T cells to a regulatory (Treg) phenotype.

It was unknown a priori which combinations of immunostimulants and inhibitors would enable selective priming and expansion of regulatory T cells. Therefore, tolerance vaccine compositions with different immunostimulants and inhibitor combinations were screened for their capacity to induce regulatory T cells in vitro. In brief, tolerance vaccines comprising nanoparticles further comprising the amphiphile Compound 285 and the peptide antigen conjugate Compound 282 at a 1:1 molar ratio were formulated with either no immunostimulant or an immunostimulant (and molar ratio of peptide antigen to immunostimulant) selected from muramyl dipeptide (MDP, 1:1 molar ratio), trehalose dibehenate (TDB, 1:0.1 molar ratio), monophosphoryl lipid A (MPL A, 1:0.25 molar ratio), liposaccharide (LPS, 1:0.25 molar ratio), Compound 36 (2BXy, 1:0.5 molar ratio), or two immunostimulants, i.e., TDB (1:0.1) and LPS (1:0.25), or two immunostimulants and an aryl hydrocarbon receptor agonist, ITE (1:0.2 molar ratio). These 8 compositions were either used directly (without inhibitor) or were combined with inhibitors selected from Torin-1, rapamycin and SR1555 at a molar ratio of total peptide antigen conjugate to drug of 1:0.1, 1:0.25 or 1:1, to yield 96 unique vaccine formulations. Note: the inhibitor may be referred to as D and the immunostimulant as D2, except when the inhibitor is absent than the immunostimulant may be referred to as D. Each of the vaccine formulations was added to a co-culture of 20,000 CD11c+ APCs and 50,000 OT-II cells specific for the CD4 T cell epitope present in Compound 282 at a final concentration of 500 nM of peptide antigen conjugate. After three days of incubation at 37C, 5% C₀₂, the T cells were evaluated by flow cytometry for the number of antigen-specific Tbet+ (Th1) and FOXP3+(Treg) present in each culture condition.

A striking finding was that the number and proportion of Tregs induced was significantly higher for the vaccines comprising rapamycin and Torin-1 as compared to those without the inhibitors (FIGS. 25 and 26). Notably, the proportion of Tregs induced with vaccine formulations with Torin-1 was significantly higher than the vaccine formulations with rapamycin, with vaccine formulations with Torin-1 inducing about a >2-4-fold higher proportion of Tregs (i.e., Treg/Tbet) as compared with formulations with rapamycin.

A highly unexpected finding was that, among the vaccine formulations with the inhibitor Torin 1, only the TLR-7/8 agonist, 2BXy, induced a signigicantly higher number of regulatory T cells (FIG. 26). This is an unexpected finding because the TLR-7/8a induces both pro-inflammatory cytokines (e.g., IL-6 and IL-12) and Type-I IFNs and is considered among the most potent adjuvants for inducing cellular immunity. Thus, it was unexpected that such a potent adjuvant could induce Tregs when used in combination with an ATP-competitive mTOR inhibitor, i.e., Torin-1. Importantly, when the TLR-7/8a 2BXy was used in combination with rapamycin, the proportion of Tregs induced was significantly lower than all other groups that included rapamycin, suggesting that inhibition of mTORC1 alone with rapamycin is not sufficient to divert T cells to Treg phenotype in the present of a potent adjuvant, such as TLR-7/8a. In contrast, inhibiting both mTORC1 and mTORC2 with Torin-1 markedly improved Treg skewing even in the presence of a potent adjuvant.

An additional notable finding was that the ratio of peptide antigen conjugate to inhibitor drug molecule also influenced the magnitude and proportion of Tregs induced. Accordingly, a 1:0.1 molar ratio of peptide antigen conjugate to drug molecule was sub-optimal in terms of Treg skewing capacity, but ratios of 1:1 or higher were found to cause cytotoxicity and/or particle size instability. Therefore, ratios between 1:0.1 and 1:1, e.g., between 1:0.25 and 1:0.5 were generally preferred.

Based on these findings, preferred compositions of tolerance vaccines comprise an ATP-competitive mTOR inhibitor at a peptide antigen conjugate to drug (i.e., inhibitor) ratio of between about 1:0.1 to about 1:1, which was found to markedly improve the proportion of Tregs induced. Such formulations may optionally comprise a second drug molecule preferably selected from immunostimulants that induce Type-I IFNs, which includes agonists of TLR-3, TLR-7, TLR-8, TLR-7/8, TLR-9 and STING.

### Example 8: Additional Amphiphiles of Formula S-B-[U]-H

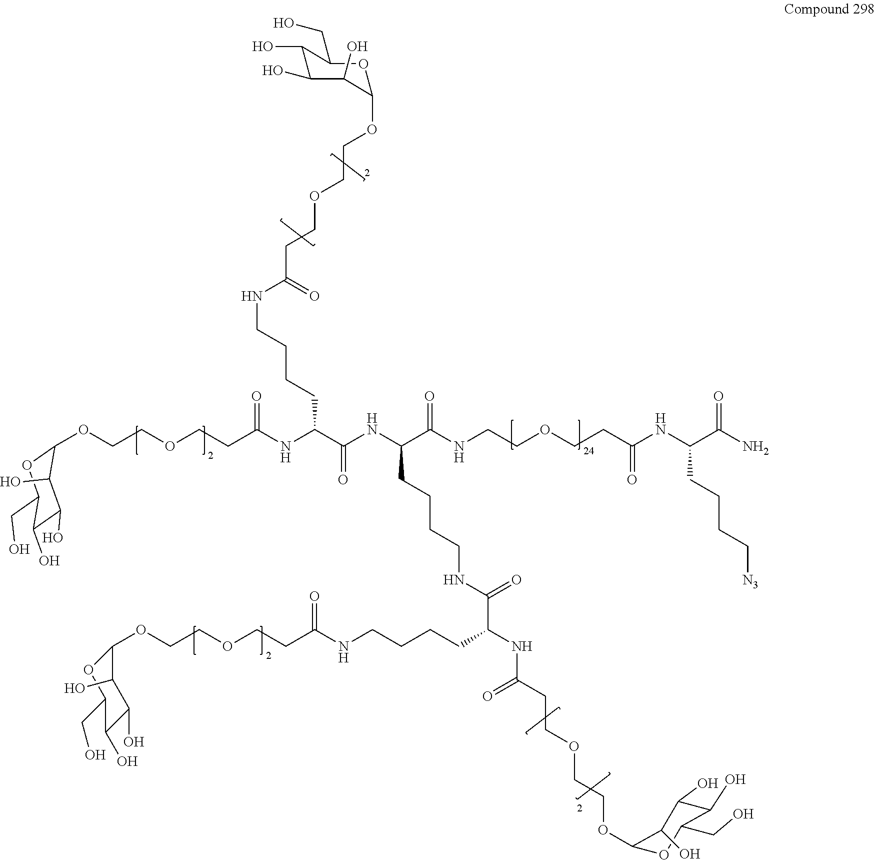

Compound 298, referred to as (Mannose-PEG₃)₄K2K-PEG₂₄-X or Tetra(Mannose-PEG₃)₄K2K-PEG₂₄-X, where X is azidolysine, was produced by solid phase peptide synthesis using a-Mannose-PEG3-acid (CarboSynUSA (San Diego, CA)). The purity and identity were confirmed by HPLC and mass spectroscopy. MS (ESI) calculated for C₁₂₇H₂₃₈N₁₂O₆₅ m/z 2971.6.

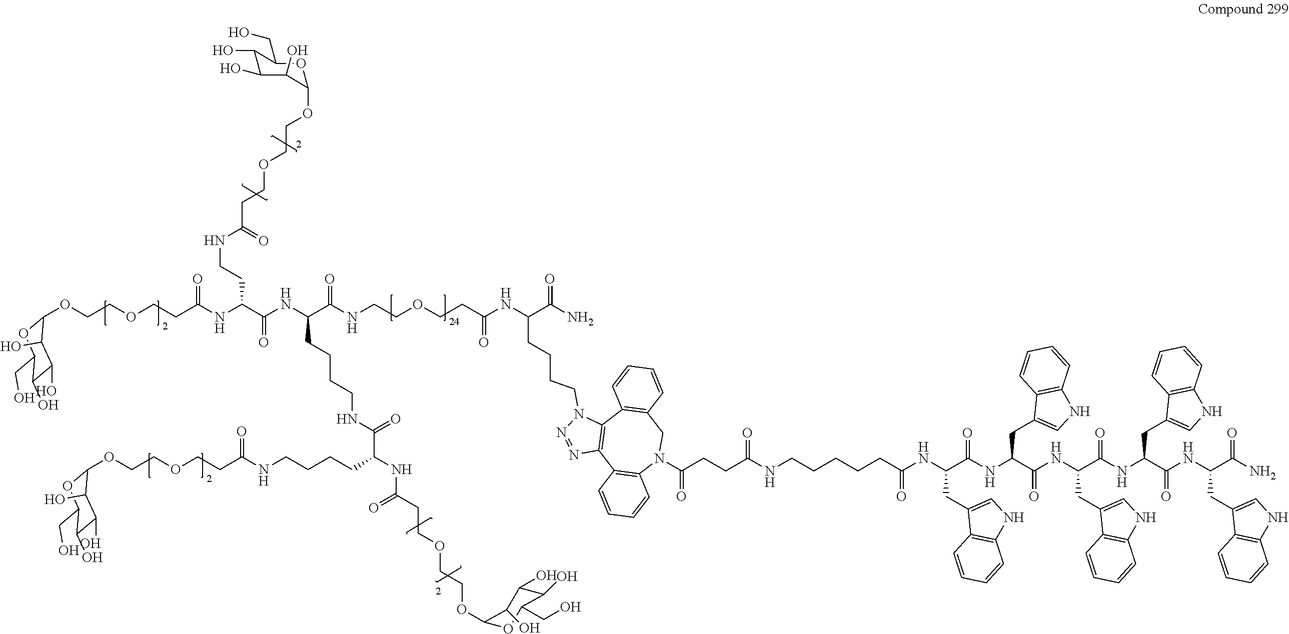

Compound 299, referred to as (Mannose-PEG₃)₄K2K-PEG₂₄-(X-DBCO)-Ahx-W₅ or Tetra(Mannose-PEG₃)₄K2K-PEG₂₄-(X-DBCO)-Ahx-W₅ was synthesized by reacting 1 equivalent of Compound 298 with 1 equivalent of Compound 62 in anhydrous DMSO for 16 hours at room temperature. HPLC was monitored to evaluate reaction progress and indicated complete conversion of Compound 298 to Compound 299, resulting in a spectroscopically pure colorless solution. MS (ESI) calculated for C₂₀₇H₃₁₅N₂₅O₇₃ m/z 4319.2.

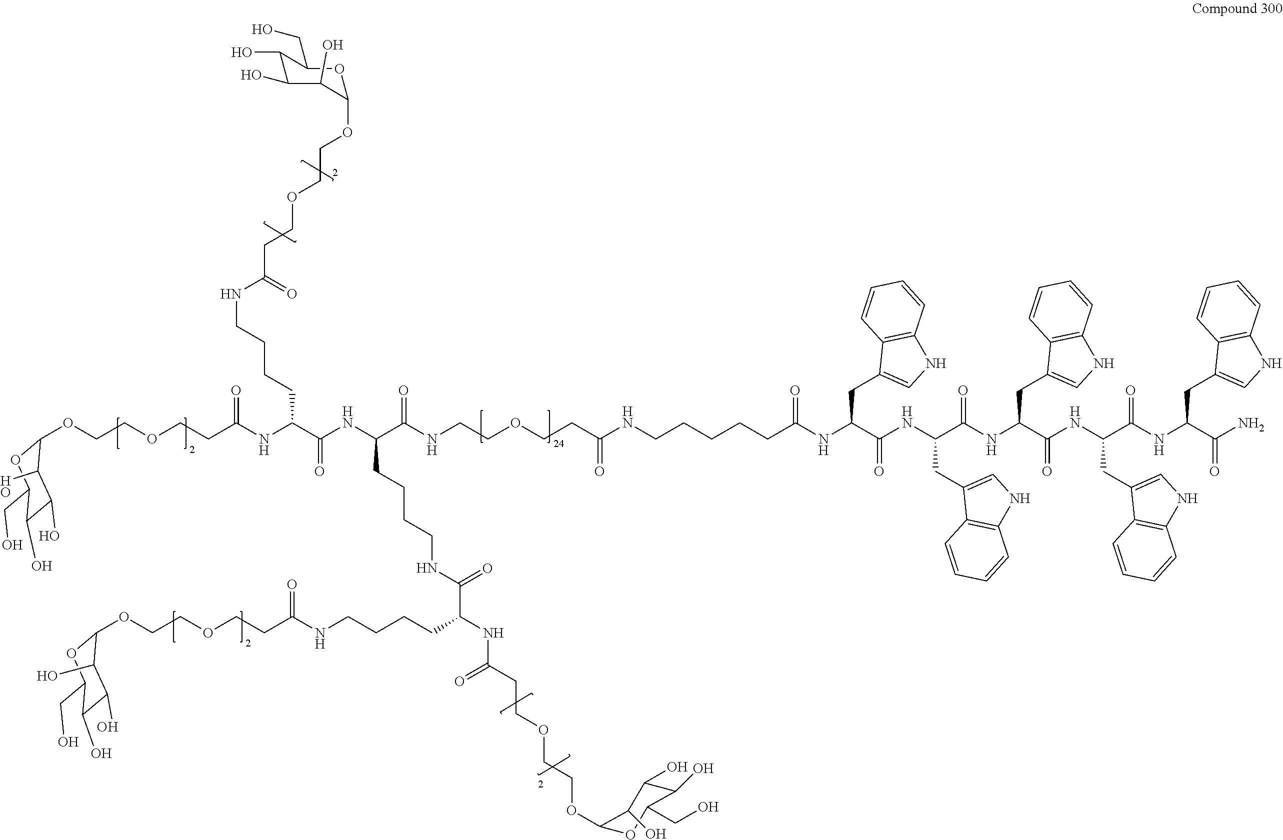

Compound 300 referred to as (Mannose-PEG₃)₄K2K-PEG₂₄-Ahx-W₅ or Tetra(Mannose-PEG₃)₄K2K-PEG₂₄-Ahx-W₅, was produced by solid phase peptide synthesis using a-Mannose-PEG3-acid (CarboSynUSA (San Diego, CA)). The purity and identity were confirmed by HPLC and mass spectroscopy. MS (ESI) calculated for C₁₈₂H₂₈₉N₁₉O₇₀ m/z 3861.0.

### Example 9: Impact of Extensions (E1 or E2) on Antibody Responses

To evaluate the impact of extensions (E1 or E2) on antibody responses, mice were immunized with vaccines comprising nanoparticle micelles further comprising a model peptide antigen derived from SARS-CoV2 spike protein (TESNKKFLPFQQFGRDIA (SEQ ID NO: 253), linked to a hydrophobic block (H) through an extension (E1) selected from either PEG12, (GGGS)₃, (AP)₇ or a heptad repeat of formula (AA_{H}-AA_{P}-AA_{P}-AA_{H}-AA_{P}-AA_{P}-AA_{P})ₑ, i.e., (IAALESK)₂ or (IAALKSK)₂, as summarized in Table 19 and FIG. 27A. Antibody responses were assessed from immunized mice on day 28 and endpoint titers against SARS-CoV2 spike protein are shown in FIG. 27B.

A notable finding was that extensions comprising heptad repeats, i.e., (IAALESK)₂ or (IAALKSK)₂, which are predicted to form coiled coils, led to significantly higher magnitude antibody responses against Spike protein as compared with those predicted to have lower rigidity, e.g., PEG12 and (GGGS)ₙ (FIG. 27B).

TABLE 19
Additional peptide antigen conjugates and amphiphiles

| | [S]-[E1]-A-[E2]-U1, U1-[E1]-A-[E2]-[S] or | U2-H-[(D)] | | |
|---|---|---|---|---|
| | S-B-U1 | | | |
| Cmpd | Product | | Theo | Found |
| # | [S]-[E1]-A-[E2]-U-H-[(D)], [(D)]-H-U-[E1]-A-[E2]-[S] or S-B-H-[(D)] | | MW | MW |
| 301 | Azp-IAALKSKIAALKSKGG-TESNKKFLPFQQFGRDIA | Compound 136 | 5844.53 | 1462 |
| | (SEQ ID NO: 456) | | | (M/4 + H)⁺ |
| | 2B3W2-Ahx-(DBCO-N3)-Azp-IAALKSKIAALKSKGG- | | | |
| | TESNKKFLPFQQFGRDIA | | | |
| 302 | Azp-IAALESEIAALESE (SEQ ID NO: 457) | Compound 62 | 2918.9 | 730 |
| | WWWWW-Ahx-(DBCO-N3)-IAALESEIAALESE | | | (M/4 + H)⁺ |
| 303 | Compound 148 | Compound 62 | 3345.5 | 1462 |
| | (OH-ethyl)₄-PEG₂₄-(N3-DBCO)-Ahx-WWWWW | | | (M/4 + H)⁺ |
| 304 | AKFVAAWTLKAAA-SPVZX (SEQ ID NO: 458) | Compound 62 | 3290.5 | 836.8 |
| | AKFVAAWTLKAAASPVZ-(N3-DBCO)-AhxWWWWW | | | (M/4 + H)⁺ |
| 305 | Azp-GGGSGGGGSGGGGSG-TESNKKFLPFQQFGRDIA | Compound 136 | 5309.5 | 1328.6 |
| | (SEQ ID NO: 459) | | | (M/4 + H)⁺ |
| | 2B3W2-Ahx-(DBCO-N3)-GGGSGGGGSGGGGSG- | | | |
| | TESNKKFLPFQQFGRDIA | | | |
| 306 | Azp-APAPAPAPAPAPAPGG-TESNKKFLPFQQFGRDIA | Compound 136 | 5598.0 | 1400.4 |
| | (SEQ ID NO: 460) | | | (M/4 + H)⁺ |
| | 2B3W2-Ahx-(DBCO-N3)-APAPAPAPAPAPAPGG- | | | |
| | TESNKKFLPFQQFGRDIA | | | |
| 307 | Azp-IAALESKIAALESKGG-TESNKKFLPFQQFGRDIA | Compound 136 | 5846.4 | 1462.6 |
| | (SEQ ID NO: 461) | | | (M/4 + H)⁺ |
| | 2B3W2-Ahx-(DBCO-N3)-IAALESKIAALESKGG- | | | |
| | TESNKKFLPFQQFGRDIA | | | |

To evaluate how the composition of the group joining the peptide antigen (A) to the heptad repeat impacts antibody responses, the model antigen derived from SARS-CoV2 spike protein, TESNKKFLPFQQFGRDIA (SEQ ID NO:253), was linked to a model heptad repeat, JAALESK-JAALESK, either directly, or via GG, PEG4, PEG12, AP, or APAPAP, sequences. As alternative embodiments, the peptide antigen was alternatively linked via a GG sequence to either a heptad repeat comprising d-amino acids (IAALESK-IAALESK), a heptad with four repeats (IAALESK-IAALESK-IAALESK-IAALESK) and a heptad repeat with alternative orientation (KSELAAI-KSELAAI) as summarized in Table 20.

TABLE 20
peptide antigen conjugates with E1 comprising heptad repeats

| Cmpd # | Peptide sequence U1-E1-A |
|---|---|
| 308 | Azide-IAALESK-IAALESK-TESNKKFLPFQQFGRDIA (SEQ ID NO: 462) |
| 309 | Azide-IAALESK-IAALESK-GG-TESNKKFLPFQQFGRDIA (SEQ ID NO: 463) |
| 310 | Azide-IAALESK-IAALESK-PEG4-TESNKKFLPFQQFGRDIA (SEQ ID NO: 464) |
| 311 | Azide-IAALESK-IAALESK-PEG12-TESNKKFLPFQQFGRDIA (SEQ ID NO: 465) |
| 312 | Azide-IAALESK-IAALESK-AP-TESNKKFLPFQQFGRDIA (SEQ ID NO: 466) |
| 313 | Azide-IAALESK-IAALESK-APAPAP-TESNKKFLPFQQFGRDIA (SEQ ID |
| | NO: 467) |
| 314 | Azide-IAALESK-IAALESK-GG-TESNKKFLPFQQFGRDIA (SEQ ID NO: 468) |
| 315 | Azide-IAALESK-IAALESK-IAALESK-IAALESK-GG-TESNKKFLPFQQFGRDIA |
| | (SEQ ID NO: 469) |
| 316 | Azide-KSELAAI-KSELAAI-GG-TESNKKFLPFQQFGRDIA (SEQ ID NO: 470) |

Note:
peptide sequences were synthesized by Genscript via solid-phase peptide synthesis. Single letter abbreviations for amino acids are used in the above table; “azide” indicates that the N-terminus is capped with an azide group (azido-pentanoic acid); PEG4 and PEG12 are PEG- based groups with 4 and 12 ethylene oxide repeats, respectively; italicized letters indicate d-amino acids; and, all sequences are amidated at the C-terminus.

As additional examples of peptide antigens linked to extensions comprising heptad repeats, peptide antigens derived from PCSK9 (Compounds 317-321) and ANGPTL3 (Compounds 322-327) were linked to extensions (E1 or E2) of varying orientation (Table 21).

TABLE 21
peptide antigen conjugates comprising minimal immunogens derived
from PCSK9 and ANGPTL3 linked to extensions (E1 or E2) comprising heptad repeats

| Cmpd # | Peptide sequence, A-E2-U1 or U1-E1-A |
|---|---|
| 317 | SIPWNLERITPPR-GG-ESKIAAL-ESKIAAL-X (SEQ ID NO: 471) |
| 318 | SIPWNLERITPPR-GG-IAALESK-IAALESK-X (SEQ ID NO: 472) |
| 319 | SIPWNLERITPPR-ESKIAAL-ESKIAAL-X (SEQ ID NO: 473) |
| 320 | SIPWNLERITPPR-GG-KSELAAI-KSELAAI-X (SEQ ID NO: 474) |
| 321 | Azide-IAALESK-IAALESK-GG-PGFLVKMSGDLLE (SEQ ID NO: 475) |
| 322 | Azide-IAALESK-IAALESK-GG-QDNSSFDSLSPEPKSRFAMLD (SEQ ID NO: 476) |
| 323 | Azide-IAALESK-IAALESK-QDNSSFDSLSPEPKSRFAMLD (SEQ ID NO: 477) |
| 324 | Ac-QDNSSFDSLSPEPKSRFAMLD-ESKIAAL-ESKIAAL-X (SEQ ID NO: 478) |
| 325 | Azide-IAALESK-IAALESK-GG-EPKSRFAMLD (SEQ ID NO: 479) |
| 326 | Azide-PEG12-RFAMLDDVKILANGLLQLGHGLKDFVHKTKGQI (SEQ ID NO: 480) |
| 327 | Azide-IAALESK-IAALESK-GG-RFAMLDDVKILANGLLQLGHGLKDFVHKTKGQI (SEQ ID |
| | NO: 481) |

Note:
peptide sequences were synthesized by Genscript via solid-phase peptide synthesis. Single letter abbreviations for amino acids are used in the above table; ″azide″ indicates that the N-terminus is capped with an azide group (azido-pentanoic acid); X = azidolysine; Ac is an acetyl group; PEG12 is a PEG-based group with 12 ethylene oxide repeats; and all sequences are amidated at the C-terminus.

As additional examples of peptide antigens linked to extensions comprising heptad repeats, peptide antigens derived from model mouse tumor neoantigens were linked to extensions E2 as summarized in Table 22.

TABLE 22
peptide antigen conjugates comprising tumor neoantigens linked to E2
comprising heptad repeats

| Cmpd # | Peptide sequence, A-E2-U1 |
|---|---|
| 328 | GRVLELFRAAQLANDVVLQIMELCGATRSPVZ-GG-ESKIAAL-ESKIAAL-X (SEQ ID NO: 482) |
| 329 | GIPVHLELASMTNMELMSSIVHQQVFPTSPVZ-GG-ESKIAAL-ESKIAAL-X (SEQ ID NO: 483) |
| 330 | GQAEPDRAHYNIVTFBBKBDSPVZ-GG-ESKIAAL-ESKIAAL-X (SEQ ID NO: 484) |
| 331 | KARDETAALLNSAVLGAAPLFVPPADSPVZ-GG-ESKIAAL-ESKIAAL-X (SEQ ID NO: 485) |

Note:
peptide sequences were synthesized by Genscript via solid-phase peptide synthesis. Single letter abbreviations for amino acids are used in the above table; B = alpha-aminobutyric acid; Z = citrulline; X = azidolysine; and all sequences are amidated at the C-terminus.

As additional examples of peptide antigens linked to extensions comprising heptad repeats, peptide antigens derived from SARS-CoV-2 were linked to extensions E1 as summarized in Table 23.

TABLE 23
peptide antigen conjugates comprising minimal immunogens derived
from SARS-COV-2 linked to E1 or E2 comprising heptad repeats

| Cmpd # | Peptide sequence, U1-E1-A |
|---|---|
| 332 | Azide-IAALESK-IAALESK-GG-PSKPSKRSFIEDLLFNKVTLADAGF (SEQ ID NO: 486) |
| 333 | Azide-IAALESK-IAALESK-GG-ARDLICAQKENGLTVLPPLL (SEQ ID NO: 487) |
| 334 | Azide-IAALESK-IAALESK-GG-LYNSASFSTFKCYGVSPTK (SEQ ID NO: 488) |
| 335 | Azide-IAALESK-IAALESK-GG-VYAWNRKRISNC (SEQ ID NO: 489) |
| 336 | Azide-IAALESK-IAALESK-GG-LVYENQKLIANQFNSAIGKIQD (SEQ ID NO: 490) |
| 337 | Azide-IAALESK-IAALESK-GG-FNSAIGKIQDSLSSTASALGKLQ (SEQ ID NO: 491) |

Note:
peptide sequences were synthesized by Genscript via solid-phase peptide synthesis. Single letter abbreviations for amino acids are used in the above table; “azide” indicates that the N-terminus is capped with an azide group (azido-pentanoic acid); and all sequences are amidated at the C-terminus.

As additional examples of peptide antigens linked to extensions comprising heptad repeats, peptide antigens derived from malaria CSP were linked to extensions E1 as summarized in Table 24.

TABLE 24
peptide antigen conjugates comprising minimal immunogens derived
from malaria linked to E1 comprising heptad repeats

| Cmpd # | Peptide sequence, U1-E1-A |
|---|---|
| 338 | Azide-IAALESK-IAALESK-GG-PADGNPDPNANPNVDPNANPNVDP (SEQ ID NO: 492) |
| 339 | Azide-IAALESK-IAALESK-GG-NANPNANPNANPNANPNANP (SEQ ID NO: 493) |
| 340 | Azide-IAALESK-IAALESK-GG-NANPNVDPNANPNVDPNANPNVDP (SEQ ID NO: 494) |

Note:
peptide sequences were synthesized by Genscript via solid-phase peptide synthesis. Single letter abbreviations for amino acids are used in the above table; “azide” indicates that the N-terminus is capped with an azide group (azido-pentanoic acid); and all sequences are amidated at the C-terminus.

### Example 10: Peptide Sequences for Activating, Priming and/or Expanding T Cells

Peptide sequences comprising peptide antigens, including the peptide antigen conjugates and peptide antigen fragments describes herein, may be used for activating, priming and/or expanding T cells in vitro or ex vivo.

The solubilizing block, S, of peptide antigen conjugates was introduced to improve solubility during manufacturing and ensure formation of nanoparticle micelles irrespective of the peptide antigen amino acid composition. The solubilizing block of peptide antigen fragments, i.e., peptide antigens optionally linked to a solubilizing block (S), extensions (E1 and/or E2) and linker precursor (U1) but not a hydrophobic block (H), could also improve the solubility of hydrophobic peptide antigens that are otherwise challenging to use to for in vitro or ex vivo activation, priming and/or expansion of T cells.

To study the impact of the solubilizing block and extensions on peptide antigen recognition by T cells in vitro, two model antigens, E7 (RAHYNIVTF) and Reps1 (AQLANDVVL), modified at the N-terminal and/or C-terminal positions were produced by solid-phase peptide synthesis (Tables 25 and 26), and then screened in vitro for T cell recognition (FIG. 28).

TABLE 25

| Compound | Figure | Sequence |
|---|---|---|
| # | group # | [S]-[E1]-A-[E2]-[U1] or [U1]-[E1]-A-[E2]-[S] |
| 341 | 3205 | RAHYNIVTFS-VZ-X (SEQ ID NO: 495) |
| 342 | 3206 | Azp-RAHYNIVTFS (SEQ ID NO: 496) |
| 343 | 3207 | Azp-VZ-RAHYNIVTFS (SEQ ID NO: 497) |
| 344 | 3208 | Azp-F′F′F′F′VZ-RAHYNIVTFS (SEQ ID NO: 498) |
| 345 | 3209 | Azp-F′F′F′F′-SPVZ-RAHYNIVTFS (SEQ ID NO: 499) |
| 346 | 3210 | Azp-F′F′F′F′-Ahx-VZ-RAHYNIVTFS (SEQ ID NO: 500) |
| 347 | 3219 | KKKKK-RAHYNIVTF (SEQ ID NO: 501) |
| 348 | 3282 | RAHYNIVTF (SEQ ID NO: 502) |
| 349 | 3283 | K-VR-RAHYNIVTF (SEQ ID NO: 503) |
| 350 | 3284 | KKKKK-VR-RAHYNIVTF (SEQ ID NO: 504) |
| 351 | 3285 | KKKKK-VR-RAHYNIVTF-SPVZ (SEQ ID NO: 505) |
| 352 | 3286 | KKKKK-VR-RAHYNIVTF-GGSPVZ (SEQ ID NO: 506) |
| 353 | 3287 | KKKKK-A′VR-RAHYNIVTF (SEQ ID NO: 507) |
| 354 | 3288 | KKKKK-SPVR-RAHYNIVTF (SEQ ID NO: 508) |
| 355 | 3289 | KKKKK-SPVB-RAHYNIVTF (SEQ ID NO: 509) |
| 356 | 3290 | KKKKK-SPVZ-RAHYNIVTF (SEQ ID NO: 510) |
| 357 | 3291 | KKKKK-SKVB-RAHYNIVTF (SEQ ID NO: 511) |
| 358 | 3292 | KKKKK-SKSB-RAHYNIVTF (SEQ ID NO: 512) |
| 359 | 3293 | KKKKK-SKVR-RAHYNIVTF (SEQ ID NO: 513) |
| 360 | 3294 | KKKKK-SKSR-RAHYNIVTF (SEQ ID NO: 514) |
| 361 | 2395 | GQAEPDRAHYNIVTFCCKCD (SEQ ID NO: 515) |

Note:
peptide sequences were synthesized by Genscript via solid-phase peptide synthesis. Single letter abbreviations for amino acids are used in the above table; “azide” indicates that the N-terminus is capped with an azide group (azido-pentanoic acid); and all sequences are amidated at the C-terminus.

TABLE 26

| Compound | Figure | |
|---|---|---|
| # | group # | Sequence |
| 362 | 3199 | AQLANDVVL-VZ-X (SEQ ID NO: 516) |
| 363 | 3200 | Azp-AQLANDVVL (SEQ ID NO: 517) |
| 364 | 3201 | Azp-VZ-AQLANDVVL (SEQ ID NO: 518) |
| 365 | 3202 | Azp-ffffVZ-AQLANDVVL (SEQ ID NO: 519) |
| 366 | 3203 | Azp-F′F′F′F′-SPVZ-AQLANDVVL (SEQ ID NO: 520) |
| 367 | 3204 | Azp-F′F′F′F′-Ahx-VZ-AQLANDVVL (SEQ ID NO: 521) |
| 368 | 3211 | GRVLELFRAAQLANDVVLQInELBGATR-SPVR (SEQ ID |
| | | NO: 522) |
| 369 | 3212 | GRVLELFRAAQLANDVVLQInELBGATR-KKKKKKKK |
| | | (SEQ ID NO: 523) |
| 370 | 3213 | GRVLELFRAAQLANDVVLQInELBGATR-SPVRA′- |
| | | KKKKKKK (SEQ ID NO: 524) |
| 371 | 3216 | KKKKK-AQLANDVVL (SEQ ID NO: 525) |
| 372 | 3269 | R-AQLANDVVL (SEQ ID NO: 526) |
| 373 | 3270 | KVR-AQLANDVVL (SEQ ID NO: 527) |
| 374 | 3271 | KKKKKVR-AQLANDVVL (SEQ ID NO: 528) |
| 375 | 3272 | KKKKKVR-AQLANDVVL-SPVZ (SEQ ID NO: 529) |
| 376 | 3273 | KKKKKVR-AQLANDVVL-GGSPVZ (SEQ ID NO: 530) |
| 377 | 3274 | KKKKK-A′VR-AQLANDVVL (SEQ ID NO: 531) |
| 378 | 3275 | KKKKK-SPVR-AQLANDVVL (SEQ ID NO: 532) |
| 379 | 3276 | KKKKK-SPVB-AQLANDVVL (SEQ ID NO: 533) |
| 380 | 3277 | KKKKK-SPVZ-AQLANDVVL (SEQ ID NO: 534) |
| 381 | 3278 | KKKKK-SKVB-AQLANDVVL (SEQ ID NO: 535) |
| 382 | 3279 | KKKKK-SKSB-AQLANDVVL (SEQ ID NO: 536) |
| 383 | 3280 | KKKKK-SKVR-AQLANDVVL (SEQ ID NO: 537) |
| 384 | 3281 | KKKKK-SKSR-AQLANDVVL (SEQ ID NO: 538) |
| 385 | 2391 | GRVLELFRAAQLANDVVLQIMELCGATR (SEQ ID NO: 539) |

Note:
peptide sequences were synthesized by Genscript via solid-phase peptide synthesis. Single letter abbreviations for amino acids are used in the above table; “azide” indicates that the N-terminus is capped with an azide group (azido-pentanoic acid); and all sequences are amidated at the C-terminus.

The results show that modification of the N-terminus of peptide antigen fragments (e.g., Compounds, 343 and 364) negatively impacts potency whereas modification of the C-terminus of peptide antigen fragments (e.g., Compounds 341 and 362) is more well tolerated (FIGS. 28A and B). Additionally, extensions comprising tetrapeptides based on alpha amino acids led to peptide antigen fragments that had higher potency for T cell recognition than those peptide antigen fragments with extensions comprising shorter peptides (e.g., dipeptides) or extensions comprising beta amino acids or epsilon amino acids (FIGS. 28A and B).

### Example 11: Use of Viral Vectors for Enhancing T Cell Responses

Adenovirus vaccines, such as ChAdOx have been used in combination with vaccinia viruses as heterologous prime-boost vaccines that have shown superior immune responses than either used alone. Therefore, it was hypothesized that combining vaccines comprising peptide antigen conjugates of the present disclosure with vaccines comprising viruses as heterologous prime-boost vaccines could result in improved T cell responses as compared with either used alone.

In a first study evaluating a heterologous prime-boost vaccine comprising a first vaccine (V1) further comprising Compound 250, a peptide antigen conjugate of formula S-E1-A-E2-U-H-D, and a second vaccine (V2) comprising adenovirus ChAdOx expressing the peptide antigen (A) with the sequence GRVLELFRAAQLANDVVLQIMELCGATR (SEQ ID NO:539), the impact of the route of the second vaccine was evaluated (FIG. 29A). A notable finding was that administering V2 by the IV route as the boost resulted in significantly higher magnitude T cell responses against the peptide antigen (A) as compared with administered V2 by the IM route (FIG. 29B). Notably, the route of both V1 comprising the peptide antigen conjugate and V2 comprising the adenovirus, ChAdOx, was found to have a major impact on the magnitude of the T cell response (FIGS. 29 and 30). Accordingly, V1 administered by the IM route followed by V2 administered by the IV route resulted in higher magnitude T cell responses as compared with both V1 and V2 administered by the IV route or both V1 and V2 administered by the IM route (FIGS. 29 and 30).

The next studies assessed how net charge of the peptide antigen conjugate and presence of amphiphile impact T cell responses when used alone as a homologous prime-boost or in combination with a viral vaccine, e.g., an adenovirus, in a heterologous-prime boost (FIG. 31A & B). The results show that reducing the net charge of the peptide antigen conjugate (from +8 to +3) and/or introducing an amphiphile do not negatively impact the magnitude of T cells induced when used as homologous prime-boost or as a heterologous prime-boost with ChAdOx (FIG. 31A & B).

A final series of studies assessed the use of adenoviruses as a biological adjuvant for use in combination with peptide antigen conjugates (FIGS. 32A &B). The results show that T cell responses induced with a peptide antigen conjugate of formula S-E1-A-E2-U-H-D comprising an adjuvant (i.e., a TLR-7/8a) can be boosted with a boost vaccine comprising a peptide antigen conjugate of formula S-E1-A-E2-U-H-D or formula S-E1-A-E2-U-H without adjuvant admixed with an adenovirus, i.e., ChAdOx that doesn't encode the antigen, A (FIGS. 32A &B). These results show that adenoviruses can provide a source of biological adjuvant when combined with peptide antigen conjugates.

1. A vaccine comprising an amphiphile having the formula S-[B]-[U]-H; and
at least one peptide antigen conjugate having the formula selected from [S]-[E1]-A-[E2]-[U]-H and H-[U]-[E1]-A-[E2]-[S],
wherein S, independently for each occurrence, is a solubilizing block;
B is a spacer;
H, independently for each occurrence, is a hydrophobic block,
wherein one or more drug molecules (D) are optionally attached to each H directly or via a suitable linker X1;
A, independently for each occurrence, is a peptide antigen;
E1, independently for each occurrence, is an N-terminal extension;
E2, independently for each occurrence, is a C-terminal extension;
U, independently for each occurrence, is a linker;
[ ] denotes that the group is optional; and
— denotes that the two adjacent groups are directly attached to one another by a covalent bond or indirectly to one another via a suitable linker X,
wherein the amphiphile and/or the at least one peptide antigen conjugate comprises a dendron amplifier.

2. The vaccine according to claim 1, wherein the S of the amphiphile comprises a dendron amplifier.

3.-6. (canceled)

7. The vaccine according to claim 1, wherein the dendron amplifier comprises repeating monomer units of 1 to 10 generations having between 2 to 6 branches per generation.

8. (canceled)

9. The vaccine according to claim 7, wherein the repeating monomer units are
(a) selected from FG1-(CH₂)_{y2}CH(R¹)₂, FG1-(CH₂)_{y2}C(R¹)₃, FG1-(CH₂CH₂O)_{y2}CH(R¹)₂, FG1-(CH₂CH₂O)_{y2}C(R¹)₃, FG1-CH(R¹)₂, and FG1-C(R¹)₃,
wherein
R¹, independently for each occurrence, is selected from (CH₂)_{y3}-FG2, (OCH₂CH₂)_{y3}-FG2, and CH₂(OCH₂CH₂)_{y3}-FG2);
y2 and y3, independently for each occurrence, are an integer of repeating units from 1 to 6;
FG1 is a first functional group; and
FG2 is a second functional group, or
(b) selected from hydroxy acids, amino acids, polyols, polyamines, and amino alcohols.

10.-12. (canceled)

13. The vaccine according to claim 1, wherein the dendron amplifier comprises a polyethylene oxide (PEG) group.

14. The vaccine according to claim 1, wherein the H of the amphiphile comprises a higher alkane, an aromatic group, a fatty acid, a sterol, a polyunsaturated hydrocarbon, squalene, saponins, or a polymer.

15.-16. (canceled)

17. The vaccine according to claim 1, wherein each H independently comprises a poly(amino acid) having the formula:
(M)ₘ-(N)ₙ—(O)ₒ—(P)ₚ—R³
wherein
each M is independently a hydrophobic amino acid;
each N is independently a reactive amino acid;
each O is independently a spacer amino acid;
each P is independently a charged amino acids;
m, n, o, and p each independently denote an integer of 10 to 100, wherein the sum of m, n, o, and p is less than or equal to 100, and at least one of m and n is not 0;
R³ is hydrogen, NH₂, NH—CH₃, NH—(CH₂)_{y5}CH₃, OH, or a D either connected directly or through a suitable linker X1; and
y5 is an integer of 1 to 6.

18.-19. (canceled)

20. The vaccine according to claim 17, wherein P, when present, is 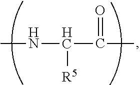
wherein each R⁵, independently, is a group that comprises 1 to 2 charged functional groups; and/or
O, when present, is 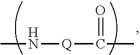
wherein each Q, independently, is selected from (CH₂)_{y6} and (CH₂CH₂O)_{y7}CH₂CH₂; each y6 is independently selected from an integer from 1 to 6; and each y7 is independently selected from an integer from 1 to 4; and/or
N, when present, is 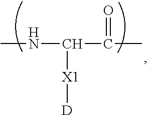
wherein each X1, independently, is a suitable linker; and each D, independently, is a drug molecule, and/or
M, when present, is 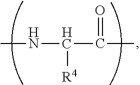
wherein each R⁴ is, independently, is 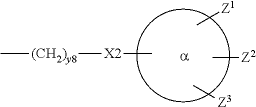
wherein, α is aryl or heteroaryl; X2 is present or absent and when present is a suitable linker; y8 is selected from an integer from 0 and 6; and Z¹, Z², and Z³ are each independently selected from hydrogen, fluorine, hydroxy, amino, alkyl, and fluoroalkyl.

21.-34. (canceled)

35. The vaccine according to claim 20, wherein each R⁴ is independently selected from:

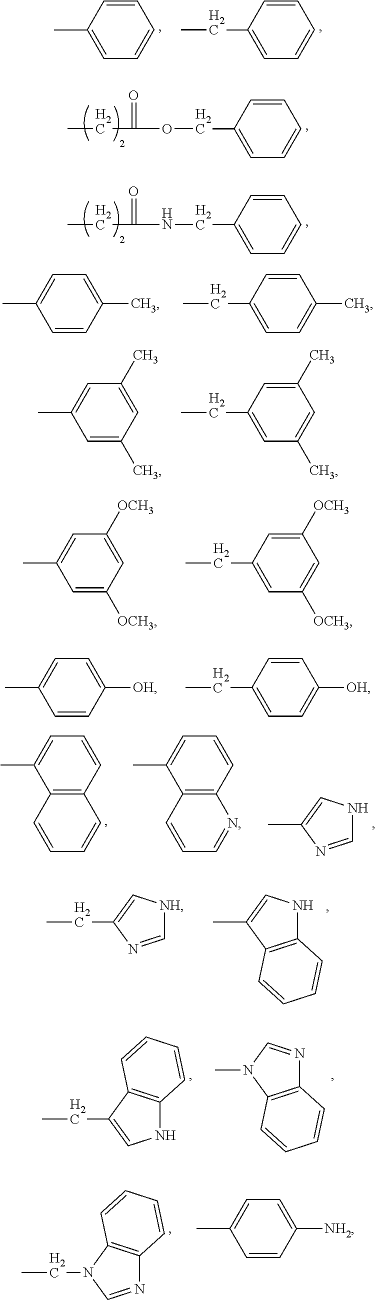

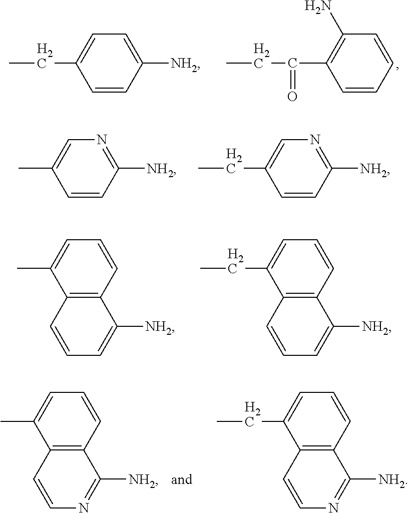

36.-37. (canceled)

38. The vaccine according to claim 20, wherein at least one D is: 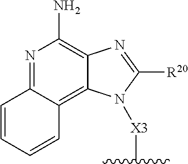
wherein
R²⁰ is selected from hydrogen, alkyl, alkoxyalkyl, aryl, heteroaryl, aminoalkyl, amide, and ester; and
X3 is selected from alkyl, alkoxyalkyl, aralkyl, heteroaralkyl, aryl, heteroaryl, and carboxy.

39.-43. (canceled)

44. The vaccine according to claim 1, wherein B, when present, is a hydrophilic polymer or peptide.

45.-50. (canceled)

51. The vaccine according to claim 1, wherein the U of the amphiphile and/or the U of the peptide antigen conjugate, when present, comprises an amide, thioether, or triazole.

52.-55. (canceled)

56. The vaccine according to claim 1, wherein at least one A comprises a sequence wherein one or more cysteine residues have been replaced with alpha amino-butyric acid and/or one or more methionine residues have been replaced with norleucine.

57.-58. (canceled)

59. A method of treating or preventing a cancer, an autoimmune disease, an allergy, an infectious disease, a cardiovascular disease, or a neurodegenerative disease in a subject in need thereof, comprising administering to the subject the vaccine of claim 1.

60. The vaccine according to claim 1, wherein the at least one peptide antigen conjugate comprises an A
(a) selected from minimal immunogens;
(b) selected from autoantigens, alloantigens, and allergens; or
(c) selected from tumor antigens.

61.-63. (canceled)

64. The vaccine according to claim 60, wherein E1 and E2 each comprise a PEG group between 4 and 36 monomeric units or E1 and E2 each comprises a peptide.

65.-74. (canceled)

75. The vaccine according to claim 73, wherein the vaccine comprises at least one D and optionally a second drug molecule (D2) and/or a third drug molecule (D3), wherein
the at least one D is each independently selected from (a) inhibitors of mTOR, RORγt, CDK8/19, and HDAC and agonists of AHR, RAR, and A₂ₐ, or (b) agonists of TLR-3, TLR-7, TLR-8, TLR-7/8, TLR-9 and STING;
D2 is selected from inhibitors of mTOR, RORγt, CDK8/19, and HDACs, agonists of AHR, RAR and A₂ₐ, and immunostimulants selected from agonists of NLRs, CLRs, TLRs and STING; and
D3 is selected from inhibitors of mTOR, RORγt, CDK8/19, and HDACs, agonists of AHR, RAR and A₂ₐ, and immunostimulants selected from agonists of NLRs, CLRs, TLRs and STING, provided that D, D2 and D3 bind to different receptors.

76.-94. (canceled)

95. A method of inducing tolerance in a subject in need thereof, comprising administering to the subject a vaccine according to claim 1,
wherein at least one A is selected from an autoantigen, alloantigen, and allergen.

96.-168. (canceled)

169. The vaccine according to claim 1, wherein S is present for at least one peptide antigen conjugate and comprises SGs selected from amines or carboxylic acids.

170. (canceled)

171. The vaccine according to claim 169, wherein the peptide antigen conjugate has a net positive charge between about +1 to about +10 at physiologic pH or between about −1 to about −10 at physiologic pH.

172.-179. (canceled)

180. A vaccine comprising at least one peptide antigen (A), wherein at least one A comprises a sequence wherein one or more cysteine residues have been replaced with alpha amino-butyric acid and/or one or more methionine residues have been replaced with norleucine.

181. The vaccine according to claim 180, wherein the vaccine further comprises
a particle delivery system selected from lipid emulsions, liposomes, PLGA particles, inorganic salt particles, and metal nanoparticles; and/or
at least one drug molecule (D) selected from immunostimulants and Treg promoting immunomodulators.

182. (canceled)

183. A vaccine comprising at least one peptide antigen conjugate having the formula selected from [S]-[E1]-A-[E2]-[U]-H and H-[U]-[E1]-A-[E2]-[S],
wherein S, independently for each occurrence, is a solubilizing block;
H, independently for each occurrence, is a hydrophobic block,
wherein one or more drug molecules (D) are optionally attached to each H directly or via a suitable linker X1;
A, independently for each occurrence, is a peptide antigen;
E1, independently for each occurrence, is an N-terminal extension;
E2, independently for each occurrence, is a C-terminal extension;
U, independently for each occurrence, is a linker;
wherein either:
(i) at least one A comprises alpha amino-butyric acid and/or norleucine;
(ii) at least one A is selected from tumor antigens, at least one D is present and is selected from agonists of TLR-7/8, and the vaccine further comprises a second drug molecule (D2) selected from inhibitors of mTOR;
(iii) at least one A is a glycopeptide; or
(iv) at least one A is selected from autoantigens, allergens and alloantigens and at least one D is present and is selected from ATP-competitive mTOR inhibitors;
[ ] denotes that the group is optional; and
— denotes that the two adjacent groups are directly attached to one another by a covalent bond or indirectly to one another via a suitable linker X.

184.-213. (canceled)

214. A vaccine comprising an expression system comprising DNA or RNA encoding for at least one peptide antigen (A), wherein the vaccine further comprises at least one drug molecule (D) selected from Treg promoting immunomodulators.

215.-218. (canceled)

219. A peptide antigen conjugate having the formula selected from S-[E1]-A-[E2]-[U]-H-[D] and [D]-H-[U]-[E1]-A-[E2]-S or a peptide antigen fragment having the formula selected from S-[E1]-A-[E2]-[U1] and [U1]-[E1]-A-[E2]-S
wherein S is a solubilizing block;
H is a hydrophobic block,
wherein one or more drug molecules (D) are optionally attached to each H directly or via a suitable linker X1;
A is a peptide antigen;
E1 is an N-terminal extension;
E2 is a C-terminal extension;
U is a linker;
U1 is a linker precursor;
[ ] denotes that the group is optional; and
— denotes that the two adjacent groups are directly attached to one another by a covalent bond or indirectly to one another via a suitable linker X, and
wherein S comprises one or more amino acids.

220.-229. (canceled)

230. A method of activating, priming and/or expanding T cells, comprising adding an aqueous solution comprising the peptide antigen conjugate or peptide antigen fragment of claim 219 to the T cells in vitro or ex vivo.

231.-252. (canceled)

253. A method of inducing an immune response in a subject in need thereof, comprising administering to the subject at least one dose of a first vaccine (V1) followed by at least one dose of a second vaccine (V2), wherein
V1 is a vaccine of claim 1; and
V2 is a viral vaccine.

254.-268. (canceled)


SEQUENCE LISTING (540 sequences)

SEQ ID NO: 1 - protein, 4 aa, Artificial Sequence
  Synthetic
GFLG

SEQ ID NO: 2 - protein, 4 aa, Artificial Sequence
  Synthetic
KPLR

SEQ ID NO: 3 - protein, 4 aa, Artificial Sequence
  Synthetic
YLLL

SEQ ID NO: 4 - protein, 4 aa, Artificial Sequence
  Synthetic
SPLR

SEQ ID NO: 5 - protein, 8 aa, Artificial Sequence
  Synthetic
KPLRYLLL

SEQ ID NO: 6 - protein, 4 aa, Artificial Sequence
  Synthetic
  misc_feature (4)..(4): Citrulline
SLVX

SEQ ID NO: 7 - protein, 4 aa, Artificial Sequence
  Synthetic
SLVL

SEQ ID NO: 8 - protein, 4 aa, Artificial Sequence
  Synthetic
  misc_feature (4)..(4): Citrulline
SPVX

SEQ ID NO: 9 - protein, 4 aa, Artificial Sequence
  Synthetic
ELVR

SEQ ID NO: 10 - protein, 4 aa, Artificial Sequence
  Synthetic
SPVR

SEQ ID NO: 11 - protein, 4 aa, Artificial Sequence
  Synthetic
SLVR

SEQ ID NO: 12 - protein, 4 aa, Artificial Sequence
  Synthetic
KPVR

SEQ ID NO: 13 - protein, 4 aa, Artificial Sequence
  Synthetic
  misc_feature (4)..(4): Citrulline
ELVX

SEQ ID NO: 14 - protein, 4 aa, Artificial Sequence
  Synthetic
ELVL

SEQ ID NO: 15 - protein, 4 aa, Artificial Sequence
  Synthetic
  misc_feature (4)..(4): Citrulline
EPVX

SEQ ID NO: 16 - protein, 4 aa, Artificial Sequence
  Synthetic
  misc_feature (4)..(4): Citrulline
KPVX

SEQ ID NO: 17 - protein, 6 aa, Artificial Sequence
  Synthetic
GGKLVR

SEQ ID NO: 18 - protein, 6 aa, Artificial Sequence
  Synthetic
GGKPLR

SEQ ID NO: 19 - protein, 6 aa, Artificial Sequence
  Synthetic
GGSLVR

SEQ ID NO: 20 - protein, 6 aa, Artificial Sequence
  Synthetic
  misc_feature (6)..(6): Citrulline
GGSLVX

SEQ ID NO: 21 - protein, 6 aa, Artificial Sequence
  Synthetic
  misc_feature (6)..(6): Citrulline
GGSPVX

SEQ ID NO: 22 - protein, 6 aa, Artificial Sequence
  Synthetic
GGSLVL

SEQ ID NO: 23 - protein, 6 aa, Artificial Sequence
  Synthetic
GGELVR

SEQ ID NO: 24 - protein, 6 aa, Artificial Sequence
  Synthetic
GGELVL

SEQ ID NO: 25 - protein, 5 aa, Artificial Sequence
  Synthetic
GSLVR

SEQ ID NO: 26 - protein, 5 aa, Artificial Sequence
  Synthetic
  misc_feature (5)..(5): Citrulline
GSLVX

SEQ ID NO: 27 - protein, 5 aa, Artificial Sequence
  Synthetic
  misc_feature (5)..(5): Citrulline
GKPVX

SEQ ID NO: 28 - protein, 5 aa, Artificial Sequence
  Synthetic
GKPVR

SEQ ID NO: 29 - protein, 5 aa, Artificial Sequence
  Synthetic
GSLVL

SEQ ID NO: 30 - protein, 5 aa, Artificial Sequence
  Synthetic
GELVL

SEQ ID NO: 31 - protein, 4 aa, Artificial Sequence
  Synthetic
  misc_feature (4)..(4): Citrulline
EGVX

SEQ ID NO: 32 - protein, 12 aa, Artificial Sequence
  Synthetic
DDDDDDDDDDDD

SEQ ID NO: 33 - protein, 11 aa, Artificial Sequence
  Synthetic
DDDDDDDDDDD

SEQ ID NO: 34 - protein, 10 aa, Artificial Sequence
  Synthetic
DDDDDDDDDD

SEQ ID NO: 35 - protein, 9 aa, Artificial Sequence
  Synthetic
DDDDDDDDD

SEQ ID NO: 36 - protein, 8 aa, Artificial Sequence
  Synthetic
DDDDDDDD

SEQ ID NO: 37 - protein, 7 aa, Artificial Sequence
  Synthetic
DDDDDDD

SEQ ID NO: 38 - protein, 6 aa, Artificial Sequence
  Synthetic
DDDDDD

SEQ ID NO: 39 - protein, 5 aa, Artificial Sequence
  Synthetic
DDDDD

SEQ ID NO: 40 - protein, 4 aa, Artificial Sequence
  Synthetic
DDDD

SEQ ID NO: 41 - protein, 12 aa, Artificial Sequence
  Synthetic
KKKKKKKKKKKK

SEQ ID NO: 42 - protein, 11 aa, Artificial Sequence
  Synthetic
KKKKKKKKKKK

SEQ ID NO: 43 - protein, 10 aa, Artificial Sequence
  Synthetic
KKKKKKKKKK

SEQ ID NO: 44 - protein, 9 aa, Artificial Sequence
  Synthetic
KKKKKKKKK

SEQ ID NO: 45 - protein, 8 aa, Artificial Sequence
  Synthetic
KKKKKKKK

SEQ ID NO: 46 - protein, 7 aa, Artificial Sequence
  Synthetic
KKKKKKK

SEQ ID NO: 47 - protein, 6 aa, Artificial Sequence
  Synthetic
KKKKKK

SEQ ID NO: 48 - protein, 5 aa, Artificial Sequence
  Synthetic
KKKKK

SEQ ID NO: 49 - protein, 4 aa, Artificial Sequence
  Synthetic
KKKK

SEQ ID NO: 50 - protein, 4 aa, Artificial Sequence
  Synthetic
KDKD

SEQ ID NO: 51 - protein, 6 aa, Artificial Sequence
  Synthetic
KDKDKD

SEQ ID NO: 52 - protein, 5 aa, Artificial Sequence
  Synthetic
LLLLL

SEQ ID NO: 53 - protein, 10 aa, Artificial Sequence
  Synthetic
LLLLLLLLLL

SEQ ID NO: 54 - protein, 5 aa, Artificial Sequence
  Synthetic
FFFFF

SEQ ID NO: 55 - protein, 10 aa, Artificial Sequence
  Synthetic
FFFFFFFFFF

SEQ ID NO: 56 - protein, 5 aa, Artificial Sequence
  Synthetic
HHHHH

SEQ ID NO: 57 - protein, 10 aa, Artificial Sequence
  Synthetic
HHHHHHHHHH

SEQ ID NO: 58 - protein, 5 aa, Artificial Sequence
  Synthetic
WWWWW

SEQ ID NO: 59 - protein, 10 aa, Artificial Sequence
  Synthetic
WWWWWWWWWW

SEQ ID NO: 60 - protein, 5 aa, Artificial Sequence
  Synthetic
  misc_feature (1)..(5): p Amino phenylalanine
XXXXX

SEQ ID NO: 61 - protein, 10 aa, Artificial Sequence
  Synthetic
  misc_feature (1)..(10): p Amino phenylalanine
XXXXXXXXXX

SEQ ID NO: 62 - protein, 20 aa, Artificial Sequence
  Synthetic
  misc_feature (1)..(20): p Amino phenylalanine
XXXXXXXXXXXXXXXXXXXX

SEQ ID NO: 63 - protein, 30 aa, Artificial Sequence
  Synthetic
  misc_feature (1)..(30): p Amino phenylalanine
XXXXXXXXXXXXXXXXXXXXXXXXXXXXXX

SEQ ID NO: 64 - protein, 10 aa, Artificial Sequence
  Synthetic
FWFWFWFWFW

SEQ ID NO: 65 - protein, 20 aa, Artificial Sequence
  Synthetic
  misc_feature (2)..(2): p Amino phenylalanine
  misc_feature (4)..(4): p Amino phenylalanine
  misc_feature (6)..(6): p Amino phenylalanine
  misc_feature (8)..(8): p Amino phenylalanine
  misc_feature (10)..(18): p Amino phenylalanine
  misc_feature (12)..(12): p Amino phenylalanine
  misc_feature (14)..(14): p Amino phenylalanine
  misc_feature (16)..(16): p Amino phenylalanine
  misc_feature (18)..(18): p Amino phenylalanine
WXWXWXWXWXWXWXWXWXWF

SEQ ID NO: 66 - protein, 5 aa, Artificial Sequence
  Synthetic
FFFFF

SEQ ID NO: 67 - protein, 5 aa, Artificial Sequence
  Synthetic
WWWWW

SEQ ID NO: 68 - protein, 5 aa, Artificial Sequence
  Synthetic
  misc_feature (1)..(5): p Amino phenylalanine
XXXXX

SEQ ID NO: 69 - protein, 10 aa, Artificial Sequence
  Synthetic
  misc_feature (1)..(10): p Amino phenylalanine
XXXXXXXXXX

SEQ ID NO: 70 - protein, 20 aa, Artificial Sequence
  Synthetic
  misc_feature (1)..(20): p Amino phenylalanine
XXXXXXXXXXXXXXXXXXXX

SEQ ID NO: 71 - protein, 7 aa, Artificial Sequence
  Synthetic
  misc_feature (7)..(7): Citrulline
KKSLVRX

SEQ ID NO: 72 - protein, 8 aa, Artificial Sequence
  Synthetic
  misc_feature (8)..(8): Citrulline
KKKSLVRX

SEQ ID NO: 73 - protein, 5 aa, Artificial Sequence
  Synthetic
EEEEE

SEQ ID NO: 74 - protein, 8 aa, Artificial Sequence
  Synthetic
  misc_feature (8)..(8): p Amino phenylalanine
KKKSLVRX

SEQ ID NO: 75 - protein, 5 aa, Artificial Sequence
  Synthetic
EEEEE

SEQ ID NO: 76 - protein, 8 aa, Artificial Sequence
  Synthetic
  misc_feature (8)..(8): p Amino phenylalanine
KKKSLVRX

SEQ ID NO: 77 - protein, 5 aa, Artificial Sequence
  Synthetic
EEEEE

SEQ ID NO: 78 - protein, 8 aa, Artificial Sequence
  Synthetic
  misc_feature (8)..(8): p Amino phenylalanine
KKKSLVRX

SEQ ID NO: 79 - protein, 5 aa, Artificial Sequence
  Synthetic
EEEEE

SEQ ID NO: 80 - protein, 10 aa, Artificial Sequence
  Synthetic
  misc_feature (1)..(10): p Amino phenylalanine
XXXXXXXXXX

SEQ ID NO: 81 - protein, 7 aa, Artificial Sequence
  Synthetic
DDDDDDD

SEQ ID NO: 82 - protein, 11 aa, Artificial Sequence
  Synthetic
  misc_feature (11)..(11): Citrulline
KKKKKKKKKKX

SEQ ID NO: 83 - protein, 8 aa, Artificial Sequence
  Synthetic
  misc_feature (8)..(8): Citrulline
KKKSLVRX

SEQ ID NO: 84 - protein, 5 aa, Artificial Sequence
  Synthetic
EEEEE

SEQ ID NO: 85 - protein, 5 aa, Artificial Sequence
  Synthetic
EEEEE

SEQ ID NO: 86 - protein, 5 aa, Artificial Sequence
  Synthetic
EWEWE

SEQ ID NO: 87 - protein, 5 aa, Artificial Sequence
  Synthetic
WEWEW

SEQ ID NO: 88 - protein, 10 aa, Artificial Sequence
  Synthetic
WWEWWWWEWW

SEQ ID NO: 89 - protein, 5 aa, Artificial Sequence
  Synthetic
WWEWW

SEQ ID NO: 90 - protein, 5 aa, Artificial Sequence
  Synthetic
EWEWE

SEQ ID NO: 91 - protein, 10 aa, Artificial Sequence
  Synthetic
EWEWEEWEWE

SEQ ID NO: 92 - protein, 10 aa, Artificial Sequence
  Synthetic
WEWEWWEWEW

SEQ ID NO: 93 - protein, 15 aa, Artificial Sequence
  Synthetic
GGEGGWGGEGGWGGE

SEQ ID NO: 94 - protein, 15 aa, Artificial Sequence
  Synthetic
GGEGGWGGEGGWGGE

SEQ ID NO: 95 - protein, 9 aa, Artificial Sequence
  Synthetic
GGEGGWGGE

SEQ ID NO: 96 - protein, 30 aa, Artificial Sequence
  Synthetic
KGWGWGKGWGWGKGWGWGKGWGWGKGWGWG

SEQ ID NO: 97 - protein, 10 aa, Artificial Sequence
  Synthetic
EEEEEEEEEE

SEQ ID NO: 98 - protein, 10 aa, Artificial Sequence
  Synthetic
KKKKKKKKKK

SEQ ID NO: 99 - protein, 10 aa, Artificial Sequence
  Synthetic
  misc_feature (1)..(10): p Amino phenylalanine
XXXXXXXXXX

SEQ ID NO: 100 - protein, 20 aa, Artificial Sequence
  Synthetic
  misc_feature (1)..(20): p Amino phenylalanine
XXXXXXXXXXXXXXXXXXXX

SEQ ID NO: 101 - protein, 20 aa, Artificial Sequence
  Synthetic
  misc_feature (1)..(20): p Amino phenylalanine
XXXXXXXXXXXXXXXXXXXX

SEQ ID NO: 102 - protein, 10 aa, Artificial Sequence
  Synthetic
  misc_feature (1)..(10): p Amino phenylalanine
XXXXXXXXXX

SEQ ID NO: 103 - protein, 5 aa, Artificial Sequence
  Synthetic
WWWWW

SEQ ID NO: 104 - protein, 10 aa, Artificial Sequence
  Synthetic
  misc_feature (10)..(10): Citrulline
SSPYSLHYLX

SEQ ID NO: 105 - protein, 27 aa, Artificial Sequence
  Synthetic
  misc_feature (27)..(27): Citrulline
DFTGSNGDPSSPYSLHYLSPTGVNEYX

SEQ ID NO: 106 - protein, 28 aa, Artificial Sequence
  Synthetic
RGYLTKILHVFHGLLPGFLVKMSGDLLE

SEQ ID NO: 107 - protein, 13 aa, Artificial Sequence
  Synthetic
PGFLVKMSGDLLE

SEQ ID NO: 108 - protein, 13 aa, Artificial Sequence
  Synthetic
  misc_feature (7)..(7): Norleucine
PGFLVKXSGDLLE

SEQ ID NO: 109 - protein, 13 aa, Artificial Sequence
  Synthetic
PGFLVKMSSDLLG

SEQ ID NO: 110 - protein, 13 aa, Artificial Sequence
  Synthetic
  misc_feature (7)..(7): Norleucine
PGFLVKXSSDLLG

SEQ ID NO: 111 - protein, 13 aa, Artificial Sequence
  Synthetic
SIPWNLERITPPR

SEQ ID NO: 112 - protein, 13 aa, Artificial Sequence
  Synthetic
SIPWNLERITPPR

SEQ ID NO: 113 - protein, 7 aa, Artificial Sequence
  Synthetic
SIPWNLE

SEQ ID NO: 114 - protein, 13 aa, Artificial Sequence
  Synthetic
SIPWNLEKVTPPR

SEQ ID NO: 115 - protein, 13 aa, Artificial Sequence
  Synthetic
SIPWNLDRVTPPR

SEQ ID NO: 116 - protein, 17 aa, Artificial Sequence
  Synthetic
NVPEEDGTRFHRQASKC

SEQ ID NO: 117 - protein, 16 aa, Artificial Sequence
  Synthetic
NVPEEDGTRFHRQASK

SEQ ID NO: 118 - protein, 7 aa, Artificial Sequence
  Synthetic
PEEDGTR

SEQ ID NO: 119 - protein, 7 aa, Artificial Sequence
  Synthetic
NVPEEDG

SEQ ID NO: 120 - protein, 16 aa, Artificial Sequence
  Synthetic
NVPEEDATRFHRQGSK

SEQ ID NO: 121 - protein, 30 aa, Artificial Sequence
  Synthetic
LFAPGEDIIGASSDCSTCFVSQSGTSQAAA

SEQ ID NO: 122 - protein, 16 aa, Artificial Sequence
  Synthetic
CSTCFVSQSGTSQAAA

SEQ ID NO: 123 - protein, 15 aa, Artificial Sequence
  Synthetic
STCFVSQSGTSQAAA

SEQ ID NO: 124 - protein, 15 aa, Artificial Sequence
  Synthetic
  misc_feature (3)..(3): alpha Aminobutyric acid
STBFVSQSGTSQAAA

SEQ ID NO: 125 - protein, 7 aa, Artificial Sequence
  Synthetic
  misc_feature (3)..(3): alpha Aminobutyric acid
STBFVSQ

SEQ ID NO: 126 - protein, 70 aa, Artificial Sequence
  Synthetic
MFTIKLLLFIVPLVISSRIDQDNSSFDSLSPEPKSRFAMLDDVKILANGLLQLGHGLKDFVHKTKGQIND

SEQ ID NO: 127 - protein, 39 aa, Artificial Sequence
  Synthetic
EPKSRFAMLDDVKILANGLLQLGHGLKDFVHKTKGQIND

SEQ ID NO: 128 - protein, 14 aa, Artificial Sequence
  Synthetic
EPKSRFAMLDDVKI

SEQ ID NO: 129 - protein, 14 aa, Artificial Sequence
  Synthetic
MLDDVKILANGLLQ

SEQ ID NO: 130 - protein, 14 aa, Artificial Sequence
  Synthetic
LANGLLQLGHGLKD

SEQ ID NO: 131 - protein, 14 aa, Artificial Sequence
  Synthetic
LGHGLKDFVHKTKG

SEQ ID NO: 132 - protein, 14 aa, Artificial Sequence
  Synthetic
LKDFVHKTKGQIND

SEQ ID NO: 133 - protein, 20 aa, Artificial Sequence
  Synthetic
RFAMLDDVKILANGLLQLGH

SEQ ID NO: 134 - protein, 20 aa, Artificial Sequence
  Synthetic
GLLQLGHGLKDFVHKTKGQI

SEQ ID NO: 135 - protein, 69 aa, Artificial Sequence
  Synthetic
IFQKLNIFDQSFYDLSLQTSEIKEEEKELRRTTYKLQVKNEEVKNMSLELNSKLESLLEEKILLQQKVK

SEQ ID NO: 136 - protein, 29 aa, Artificial Sequence
  Synthetic
MHQKRTAMFQDPQERPRKLPQLCTELQTT

SEQ ID NO: 137 - protein, 29 aa, Artificial Sequence
  Synthetic
PRKLPQLCTELQTTIHDIILECVYCKQQL

SEQ ID NO: 138 - protein, 29 aa, Artificial Sequence
  Synthetic
HDIILECVYCKQQLLRREVYDFAFRDLCI

SEQ ID NO: 139 - protein, 29 aa, Artificial Sequence
  Synthetic
RREVYDFAFRDLCIVYRDGNPYAVCDKCL

SEQ ID NO: 140 - protein, 29 aa, Artificial Sequence
  Synthetic
YRDGNPYAVCDKCLKFYSKISEYRHYCYS

SEQ ID NO: 141 - protein, 29 aa, Artificial Sequence
  Synthetic
FYSKISEYRHYCYSLYGTTLEQQYNKPLC

SEQ ID NO: 142 - protein, 29 aa, Artificial Sequence
  Synthetic
YGTTLEQQYNKPLCDLLIRCINCQKPLCP

SEQ ID NO: 143 - protein, 29 aa, Artificial Sequence
  Synthetic
LLIRCINCQKPLCPEEKQRHLDKKQRFHN

SEQ ID NO: 144 - protein, 28 aa, Artificial Sequence
  Synthetic
EKQRHLDKKQRFHNIRGRWTGRCMSCCR

SEQ ID NO: 145 - protein, 24 aa, Artificial Sequence
  Synthetic
IRGRWTGRCMSCCRSSRTRRETQL

SEQ ID NO: 146 - protein, 27 aa, Artificial Sequence
  Synthetic
MHGDTPTLHEYMLDLQPETTDLYCYEQ

SEQ ID NO: 147 - protein, 25 aa, Artificial Sequence
  Synthetic
DLQPETTDLYCYEQLNDSSEEEDEI

SEQ ID NO: 148 - protein, 25 aa, Artificial Sequence
  Synthetic
YEQLNDSSEEEDEIDGPAGQAEPDR

SEQ ID NO: 149 - protein, 27 aa, Artificial Sequence
  Synthetic
DEIDGPAGQAEPDRAHYNIVTFCCKCD

SEQ ID NO: 150 - protein, 32 aa, Artificial Sequence
  Synthetic
RAHYNIVTFCCKCDSTLRLCVQSTHVDIRTLE

SEQ ID NO: 151 - protein, 32 aa, Artificial Sequence
  Synthetic
LCVQSTHVDIRTLEDLLMGTLGIVCPICSQKP

SEQ ID NO: 152 - protein, 20 aa, Artificial Sequence
  Synthetic
QLYQTCKAAGTCPSDVIPKI

SEQ ID NO: 153 - protein, 29 aa, Artificial Sequence
  Synthetic
  misc_feature (1)..(1): Norleucine
  misc_feature (8)..(8): Norleucine
  misc_feature (23)..(23): alpha Aminobutyric acid
XHQKRTAXFQDPQERPRKLPQLBTELQTT

SEQ ID NO: 154 - protein, 29 aa, Artificial Sequence
  Synthetic
  misc_feature (1)..(1): Norleucine
  misc_feature (8)..(8): Norleucine
XHQKRTAXFQDPQERPRKLPQLCTELQTT

SEQ ID NO: 155 - protein, 29 aa, Artificial Sequence
  Synthetic
  misc_feature (23)..(23): alpha Aminobutyric acid
MHQKRTAMFQDPQERPRKLPQLBTELQTT

SEQ ID NO: 156 - protein, 29 aa, Artificial Sequence
  Synthetic
  misc_feature (8)..(8): alpha Aminobutyric acid
  misc_feature (22)..(22): alpha Aminobutyric acid
  misc_feature (25)..(25): alpha Aminobutyric acid
PRKLPQLBTELQTTIHDIILEBVYBKQQL

SEQ ID NO: 157 - protein, 29 aa, Artificial Sequence
  Synthetic
  misc_feature (7)..(7): alpha Aminobutyric acid
  misc_feature (10)..(10): alpha Aminobutyric acid
  misc_feature (28)..(28): alpha Aminobutyric acid
HDIILEBVYBKQQLLRREVYDFAFRDLBI

SEQ ID NO: 158 - protein, 29 aa, Artificial Sequence
  Synthetic
  misc_feature (13)..(13): alpha Aminobutyric acid
  misc_feature (25)..(25): alpha Aminobutyric acid
  misc_feature (28)..(28): alpha Aminobutyric acid
RREVYDFAFRDLBIVYRDGNPYAVBDKBL

SEQ ID NO: 159 - protein, 29 aa, Artificial Sequence
  Synthetic
  misc_feature (10)..(10): alpha Aminobutyric acid
  misc_feature (13)..(13): alpha Aminobutyric acid
  misc_feature (27)..(27): alpha Aminobutyric acid
YRDGNPYAVBDKBLKFYSKISEYRHYBYS

SEQ ID NO: 160 - protein, 29 aa, Artificial Sequence
  Synthetic
  misc_feature (12)..(12): alpha Aminobutyric acid
  misc_feature (29)..(29): alpha Aminobutyric acid
FYSKISEYRHYBYSLYGTTLEQQYNKPLB

SEQ ID NO: 161 - protein, 28 aa, Artificial Sequence
  Synthetic
  misc_feature (19)..(19): alpha Aminobutyric acid
  misc_feature (22)..(22): alpha Aminobutyric acid
  misc_feature (27)..(27): alpha Aminobutyric acid
YGTTLEQQYNKPLDLLIRBINBQKPLBP

SEQ ID NO: 162 - protein, 29 aa, Artificial Sequence
  Synthetic
  misc_feature (5)..(5): alpha Aminobutyric acid
  misc_feature (8)..(8): alpha Aminobutyric acid
  misc_feature (13)..(13): alpha Aminobutyric acid
LLIRBINBQKPLBPEEKQRHLDKKQRFHN

SEQ ID NO: 163 - protein, 28 aa, Artificial Sequence
  Synthetic
  misc_feature (24)..(24): Norleucine
EKQRHLDKKQRFHNIRGRWTGRCXSCCR

SEQ ID NO: 164 - protein, 28 aa, Artificial Sequence
  Synthetic
  misc_feature (23)..(23): alpha Aminobutyric acid
  misc_feature (24)..(24): Norleucine
  misc_feature (26)..(26): alpha Aminobutyric acid
  misc_feature (27)..(27): alpha Aminobutyric acid
EKQRHLDKKQRFHNIRGRWTGRBXSBBR

SEQ ID NO: 165 - protein, 28 aa, Artificial Sequence
  Synthetic
  misc_feature (23)..(23): alpha Aminobutyric acid
  misc_feature (26)..(26): alpha Aminobutyric acid
  misc_feature (27)..(27): alpha Aminobutyric acid
EKQRHLDKKQRFHNIRGRWTGRBMSBBR

SEQ ID NO: 166 - protein, 24 aa, Artificial Sequence
  Synthetic
  misc_feature (10)..(10): Norleucine
IRGRWTGRCXSCCRSSRTRRETQL

SEQ ID NO: 167 - protein, 24 aa, Artificial Sequence
  Synthetic
  misc_feature (9)..(9): alpha Aminobutyric acid
  misc_feature (10)..(10): Norleucine
  misc_feature (12)..(12): alpha Aminobutyric acid
  misc_feature (13)..(13): alpha Aminobutyric acid
IRGRWTGRBXSBBRSSRTRRETQL

SEQ ID NO: 168 - protein, 24 aa, Artificial Sequence
  Synthetic
  misc_feature (9)..(9): alpha Aminobutyric acid
  misc_feature (12)..(12): alpha Aminobutyric acid
  misc_feature (13)..(13): alpha Aminobutyric acid
IRGRWTGRBMSBBRSSRTRRETQL

SEQ ID NO: 169 - protein, 27 aa, Artificial Sequence
  Synthetic
  misc_feature (1)..(1): Norleucine
  misc_feature (12)..(12): Norleucine
XHGDTPTLHEYXLDLQPETTDLYCYEQ

SEQ ID NO: 170 - protein, 26 aa, Artificial Sequence
  Synthetic
  misc_feature (11)..(11): Norleucine
  misc_feature (23)..(23): alpha Aminobutyric acid
HGDTPTLHEYXLDLQPETTDLYBYEQ

SEQ ID NO: 171 - protein, 27 aa, Artificial Sequence
  Synthetic
  misc_feature (1)..(1): Norleucine
  misc_feature (12)..(12): Norleucine
XHGDTPTLHEYXLDLQPETTDLYMYEQ

SEQ ID NO: 172 - protein, 25 aa, Artificial Sequence
  Synthetic
  misc_feature (11)..(11): alpha Aminobutyric acid
DLQPETTDLYBYEQLNDSSEEEDEI

SEQ ID NO: 173 - protein, 25 aa, Artificial Sequence
  Synthetic
YEQLNDSSEEEDEIDGPAGQAEPDR

SEQ ID NO: 174 - protein, 27 aa, Artificial Sequence
  Synthetic
  misc_feature (23)..(23): alpha Aminobutyric acid
  misc_feature (24)..(24): alpha Aminobutyric acid
  misc_feature (26)..(26): alpha Aminobutyric acid
DEIDGPAGQAEPDRAHYNIVTFBBKBD

SEQ ID NO: 175 - protein, 32 aa, Artificial Sequence
  Synthetic
  misc_feature (10)..(10): alpha Aminobutyric acid
  misc_feature (11)..(11): alpha Aminobutyric acid
  misc_feature (13)..(13): alpha Aminobutyric acid
  misc_feature (20)..(20): alpha Aminobutyric acid
RAHYNIVTFBBKBDSTLRLBVQSTHVDIRTLE

SEQ ID NO: 176 - protein, 32 aa, Artificial Sequence
  Synthetic
  misc_feature (18)..(18): Norleucine
LCVQSTHVDIRTLEDLLXGTLGIVCPICSQKP

SEQ ID NO: 177 - protein, 32 aa, Artificial Sequence
  Synthetic
  misc_feature (2)..(2): alpha Aminobutyric acid
  misc_feature (18)..(18): Norleucine
  misc_feature (25)..(25): alpha Aminobutyric acid
  misc_feature (28)..(28): alpha Aminobutyric acid
LBVQSTHVDIRTLEDLLNGTLGIVBPIBSQKP

SEQ ID NO: 178 - protein, 32 aa, Artificial Sequence
  Synthetic
  misc_feature (2)..(2): alpha Aminobutyric acid
  misc_feature (25)..(25): alpha Aminobutyric acid
  misc_feature (28)..(28): alpha Aminobutyric acid
LBVQSTHVDIRTLEDLLMGTLGIVBPIBSQKP

SEQ ID NO: 179 - protein, 20 aa, Artificial Sequence
  Synthetic
  misc_feature (6)..(6): alpha Aminobutyric acid
  misc_feature (12)..(12): alpha Aminobutyric acid
QLYQTBKAAGTBPSDVIPKI

SEQ ID NO: 180 - protein, 29 aa, Artificial Sequence
  Synthetic
ALQAIELQLTLETIYNSQYSNEKWTLQDV

SEQ ID NO: 181 - protein, 29 aa, Artificial Sequence
  Synthetic
NSQYSNEKWTLQDVSLEVYLTAPTGCIKK

SEQ ID NO: 182 - protein, 29 aa, Artificial Sequence
  Synthetic
SVTVVEGQVDYYGLYYVHEGIRTYFVQFK

SEQ ID NO: 183 - protein, 32 aa, Artificial Sequence
  Synthetic
LKGDANTLKCLRYRFKKHCTLYTAVSSTWHWT

SEQ ID NO: 184 - protein, 29 aa, Artificial Sequence
  Synthetic
KHKSAIVTLTYDSEWQRDQFLSQVKIPKT

SEQ ID NO: 185 - protein, 29 aa, Artificial Sequence
  Synthetic
MHQKRTAMFQDPQERPRKLPQLCTELQTT

SEQ ID NO: 186 - protein, 29 aa, Artificial Sequence
  Synthetic
PRKLPQLCTELQTTIHDIILECVYCKQQL

SEQ ID NO: 187 - protein, 29 aa, Artificial Sequence
  Synthetic
HDIILECVYCKQQLLRREVYDFAFRDLCI

SEQ ID NO: 188 - protein, 29 aa, Artificial Sequence
  Synthetic
RREVYDFAFRDLCIVYRDGNPYAVCDKCL

SEQ ID NO: 189 - protein, 29 aa, Artificial Sequence
  Synthetic
YRDGNPYAVCDKCLKFYSKISEYRHYCYS

SEQ ID NO: 190 - protein, 29 aa, Artificial Sequence
  Synthetic
FYSKISEYRHYCYSLYGTTLEQQYNKPLC

SEQ ID NO: 191 - protein, 29 aa, Artificial Sequence
  Synthetic
YGTTLEQQYNKPLCDLLIRCINCQKPLCP

SEQ ID NO: 192 - protein, 31 aa, Artificial Sequence
  Synthetic
CPEEKQRHLDKKQRFHNIRGRWTGRCMSCCR

SEQ ID NO: 193 - protein, 27 aa, Artificial Sequence
  Synthetic
MHGDTPTLHEYMLDLQPETTDLYCYEQ

SEQ ID NO: 194 - protein, 29 aa, Artificial Sequence
  Synthetic
AGQAEPDRAHYNIVTFCCKCDSTLRLCVQ

SEQ ID NO: 195 - protein, 32 aa, Artificial Sequence; identical to SEQ ID NO: 151
  Synthetic

SEQ ID NO: 196 - protein, 29 aa, Artificial Sequence
  Synthetic
  misc_feature (26)..(26): alpha Aminobutyric acid
NSQYSNEKWTLQDVSLEVYLTAPTGBIKK

SEQ ID NO: 197 - protein, 32 aa, Artificial Sequence
  Synthetic
  misc_feature (10)..(10): alpha Aminobutyric acid
  misc_feature (19)..(19): alpha Aminobutyric acid
LKGDANTLKBLRYRFKKHBTLYTAVSSTWHWT

SEQ ID NO: 198 - protein, 29 aa, Artificial Sequence
  Synthetic
  misc_feature (1)..(1): Norleucine
  misc_feature (8)..(8): Norleucine
  misc_feature (23)..(23): alpha Aminobutyric acid
XHQKRTAXFQDPQERPRKLPQLBTELQTT

SEQ ID NO: 199 - protein, 29 aa, Artificial Sequence
  Synthetic
  misc_feature (8)..(8): alpha Aminobutyric acid
  misc_feature (22)..(22): alpha Aminobutyric acid
  misc_feature (25)..(25): alpha Aminobutyric acid
PRKLPQLBTELQTTIHDIILEBVYBKQQL

SEQ ID NO: 200 - protein, 29 aa, Artificial Sequence
  Synthetic
  misc_feature (7)..(7): alpha Aminobutyric acid
  misc_feature (10)..(10): alpha Aminobutyric acid
  misc_feature (28)..(28): alpha Aminobutyric acid
HDIILEBVYBKQQLLRREVYDFAFRDLBI

SEQ ID NO: 201 - protein, 29 aa, Artificial Sequence
  Synthetic
  misc_feature (13)..(13): alpha Aminobutyric acid
  misc_feature (25)..(25): alpha Aminobutyric acid
  misc_feature (28)..(28): alpha Aminobutyric acid
RREVYDFAFRDLBIVYRDGNPYAVBDKBL

SEQ ID NO: 202 - protein, 29 aa, Artificial Sequence
  Synthetic
  misc_feature (10)..(10): alpha Aminobutyric acid
  misc_feature (13)..(13): alpha Aminobutyric acid
  misc_feature (27)..(27): alpha Aminobutyric acid
YRDGNPYAVBDKBLKFYSKISEYRHYBYS

SEQ ID NO: 203 - protein, 29 aa, Artificial Sequence
  Synthetic
  misc_feature (12)..(12): alpha Aminobutyric acid
  misc_feature (29)..(29): alpha Aminobutyric acid
FYSKISEYRHYBYSLYGTTLEQQYNKPLB

SEQ ID NO: 204 - protein, 29 aa, Artificial Sequence
  Synthetic
  misc_feature (14)..(14): alpha Aminobutyric acid
  misc_feature (20)..(20): alpha Aminobutyric acid
  misc_feature (23)..(23): alpha Aminobutyric acid
  misc_feature (28)..(28): alpha Aminobutyric acid
YGTTLEQQYNKPLBDLLIRBINBQKPLBP

SEQ ID NO: 205 - protein, 31 aa, Artificial Sequence
  Synthetic
  misc_feature (1)..(1): alpha Aminobutyric acid
  misc_feature (26)..(26): alpha Aminobutyric acid
  misc_feature (27)..(27): Norleucine
  misc_feature (29)..(29): alpha Aminobutyric acid
  misc_feature (30)..(30): alpha Aminobutyric acid
BPEEKQRHLDKKQRFHNIRGRWTGRBXSBBR

SEQ ID NO: 206 - protein, 27 aa, Artificial Sequence
  Synthetic
  misc_feature (1)..(1): Norleucine
  misc_feature (12)..(12): Norleucine
  misc_feature (24)..(24): alpha Aminobutyric acid
XHGDTPTLHEYXLDLQPETTDLYBYEQ

SEQ ID NO: 207 - protein, 29 aa, Artificial Sequence
  Synthetic
  misc_feature (17)..(17): alpha Aminobutyric acid
  misc_feature (18)..(18): alpha Aminobutyric acid
  misc_feature (20)..(20): alpha Aminobutyric acid
  misc_feature (27)..(27): alpha Aminobutyric acid
AGQAEPDRAHYNIVTFBBKBDSTLRLBVQ

SEQ ID NO: 208 - protein, 32 aa, Artificial Sequence
  Synthetic
  misc_feature (2)..(2): alpha Aminobutyric acid
  misc_feature (18)..(18): Norleucine
  misc_feature (25)..(25): alpha Aminobutyric acid
  misc_feature (28)..(28): alpha Aminobutyric acid
LBVQSTHVDIRTLEDLLXGTLGIVBPIBSQKP

SEQ ID NO: 209 - protein, 244 aa, Artificial Sequence
  Synthetic
APLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 210 - protein, 35 aa, Artificial Sequence
  Synthetic
CGGVLVHPQWVLTAAHCIRNKSVILLGRHSLFHPE

SEQ ID NO: 211 - protein, 35 aa, Artificial Sequence
  Synthetic
SLFHPEDTGQVFQVSHSFPHPLYDMSLLKNRFLRP

SEQ ID NO: 212 - protein, 29 aa, Artificial Sequence
  Synthetic
PCALPERPSLYTKVVHYRKWIKDTIVANP

SEQ ID NO: 213 - protein, 364 aa, Artificial Sequence
  Synthetic
FFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 214 - protein, 35 aa, Artificial Sequence
  Synthetic
RTLMSAMTNLAALFPPEGVSIWNPILLWQPIPVHT

SEQ ID NO: 215 - protein, 35 aa, Artificial Sequence
  Synthetic
PILLWQPIPVHTVPLSEDQLLYLPFRNCPRFQELE

SEQ ID NO: 216 - protein, 35 aa, Artificial Sequence
  Synthetic
ATEDTMTKLRELSELSLLSLYGIHKQKEKSRLQGG

SEQ ID NO: 217 - protein, 35 aa, Artificial Sequence
  Synthetic
LQGGVLVNEILNHMKRATQIPSYKKLIMYSAHDTT

SEQ ID NO: 218 - protein, 35 aa, Artificial Sequence
  Synthetic
MALDVYNGLLPPYASCHLTELYFEKGEYFVEMYYR

SEQ ID NO: 219 - protein, 35 aa, Artificial Sequence
  Synthetic
YFEKGEYFVEMYYRNETQHEPYPLMLPGCSPSCPL

SEQ ID NO: 220 - protein, 338 aa, Artificial Sequence
  Synthetic
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVLIFKSILFLPCLRKKILKIRHGWEDVTKINKTEICSQL

SEQ ID NO: 221 - protein, 35 aa, Artificial Sequence
  Synthetic
LFPQWHLPIKIAAIIASLTFLYTLLREVIHPLATS

SEQ ID NO: 222 - protein, 35 aa, Artificial Sequence
  Synthetic
YTLLREVIHPLATSHQQYFYKIPILVINKVLPMVS

SEQ ID NO: 223 - protein, 38 aa, Artificial Sequence
  Synthetic
RKQFGLLSFFFAVLHAIYSLSYPMRRSYRYKLLNWAYQ

SEQ ID NO: 224 - protein, 35 aa, Artificial Sequence
  Synthetic
EDAWIEHDVWRMEIYVSLGIVGLAILALLAVTSIP

SEQ ID NO: 225 - protein, 35 aa, Artificial Sequence
  Synthetic
LAVTSIPSVSDSLTWREFHYIQSKLGIVSLLLGTI

SEQ ID NO: 226 - protein, 35 aa, Artificial Sequence
  Synthetic
DIKQFVWYTPPTFMIAVFLPIVLIFKSILFLPCLR

SEQ ID NO: 227 - protein, 389 aa, Artificial Sequence
  Synthetic
SSPTSSASSFSSSAPFLASAVSAQPPLPDQCPALCECSEAARTVKCVNRNLTEVPTDLPAYVRNLFLTGNQLAVLPAGAFARRPPLAELAALNLSGSRLDEVRAGAFEHLPSLRQLDLSHNPLADLSPFAFSGSNASVSAPSPLVELILNHIVPPEDERQNRSFEGMVVAALLAGRALQGLRRLELASNHFLYLPRDVLAQLPSLRHLDLSNNSLVSLTYVSFRNLTHLESLHLEDNALKVLHNGTLAELQGLPHIRVFLDNNPWVCDCHMADMVTWLKETEVVQGKDRLTCAYPEKMRNRVLLELNSADLDCDPILPPSLQTSYVFLGIVLALIGAIFLLVLYLNRKGIKKWMHNIRDACRDHMEGYHYRYEINADPRLTNLSSNSDV

SEQ ID NO: 228 - protein, 35 aa, Artificial Sequence
  Synthetic
SPTSSASSFSSSAPFLASAVSAQPPLPDQCPALCE

SEQ ID NO: 229 - protein, 35 aa, Artificial Sequence
  Synthetic
RNLTEVPTDLPAYVRNLFLTGNQLAVLPAGAFARR

SEQ ID NO: 230 - protein, 35 aa, Artificial Sequence
  Synthetic
ALQGLRRLELASNHFLYLPRDVLAQLPSLRHLDLS

SEQ ID NO: 231 - protein, 35 aa, Artificial Sequence
  Synthetic
LSNNSLVSLTYVSFRNLTHLESLHLEDNALKVLHN

SEQ ID NO: 232 - protein, 35 aa, Artificial Sequence
  Synthetic
DCDPILPPSLQTSYVFLGIVLALIGAIFLLVLYLN

SEQ ID NO: 233 - protein, 11 aa, Artificial Sequence
  Synthetic
RNNILRTQESE

SEQ ID NO: 234 - protein, 17 aa, Artificial Sequence
  Synthetic
LNDKHSNGTIKDRSPYR

SEQ ID NO: 235 - protein, 12 aa, Artificial Sequence
  Synthetic
SWRNNILRTQES

SEQ ID NO: 236 - protein, 9 aa, Artificial Sequence
  Synthetic
DNWHGSNRP

SEQ ID NO: 237 - protein, 11 aa, Artificial Sequence
  Synthetic
DNPRPNDKTGS

SEQ ID NO: 238 - protein, 14 aa, Artificial Sequence
  Synthetic
DPNGWTGTDNNFSI

SEQ ID NO: 239 - protein, 9 aa, Artificial Sequence
  Synthetic
QLDPAFRAG

SEQ ID NO: 240 - protein, 9 aa, Artificial Sequence
  Synthetic
RGLYFPAGL

SEQ ID NO: 241 - protein, 11 aa, Artificial Sequence
  Synthetic
STGPCRTCMTK

SEQ ID NO: 242 - protein, 9 aa, Artificial Sequence
  Synthetic
AVGIGAVFL

SEQ ID NO: 243 - protein, 30 aa, Artificial Sequence
  Synthetic
EINCTRPNNNTRPGEIIGDIRQAHCNISRA

SEQ ID NO: 244 - protein, 23 aa, Artificial Sequence
  Synthetic
YNKRKRIHIGPGRAFYTTKNIIG

SEQ ID NO: 245 - protein, 15 aa, Artificial Sequence
  Synthetic
PADGNPDPNANPNVD

SEQ ID NO: 246 - protein, 15 aa, Artificial Sequence
  Synthetic
NPDPNANPNVDPNAN

SEQ ID NO: 247 - protein, 15 aa, Artificial Sequence
  Synthetic
NANPNVDPNANPNVD

SEQ ID NO: 248 - protein, 15 aa, Artificial Sequence
  Synthetic
NANPNANPNANPNAN

SEQ ID NO: 249 - protein, 12 aa, Artificial Sequence
  Synthetic
DPNANPNVDPNA

SEQ ID NO: 250 - protein, 16 aa, Artificial Sequence
  Synthetic
KQPADGNPDPNANPNV

SEQ ID NO: 251 - protein, 35 aa, Artificial Sequence
  Synthetic
EDNEKLRKPKHKKLKQPADGNPDPNANPNVDPNAN

SEQ ID NO: 252 - protein, 20 aa, Artificial Sequence
  Synthetic
KLRKPKHKKLKQPADGNPDP

SEQ ID NO: 253 - protein, 18 aa, Artificial Sequence
  Synthetic
TESNKKFLPFQQFGRDIA

SEQ ID NO: 254 - protein, 31 aa, Artificial Sequence
  Synthetic
SQILPDPSKPSKRSFIEDLLFNKVTLADAGF

SEQ ID NO: 255 - protein, 25 aa, Artificial Sequence
  Synthetic
SQILPDPSKPSKRSFIEDLLFNKVT

SEQ ID NO: 256 - protein, 25 aa, Artificial Sequence
  Synthetic
PSKPSKRSFIEDLLFNKVTLADAGF

SEQ ID NO: 257 - protein, 31 aa, Artificial Sequence
  Synthetic
DYSVLYNSASFSTFKCYGVSPTKLNDLCFTN

SEQ ID NO: 258 - protein, 20 aa, Artificial Sequence
  Synthetic
LYNSASFSTFKCYGVSPTKL

SEQ ID NO: 259 - protein, 25 aa, Artificial Sequence
  Synthetic
SNNLDSKVGGNYNYLYRLFRKSNLK

SEQ ID NO: 260 - protein, 25 aa, Artificial Sequence
  Synthetic
YRLFRKSNLKPFERDISTEIYQAGS

SEQ ID NO: 261 - protein, 25 aa, Artificial Sequence
  Synthetic
ISTEIYQAGSTPCNGVEGFNCYFPL

SEQ ID NO: 262 - protein, 24 aa, Artificial Sequence
  Synthetic
VEGFNCYFPLQSYGFQPTNGVGYQ

SEQ ID NO: 263 - protein, 58 aa, Artificial Sequence
  Synthetic
SNNLDSKVGGNYNYLYRLFRGSGIYQAGSTPCNGVEGFNCYFPLQSYGFQPTNGVGYQ

SEQ ID NO: 264 - protein, 247 aa, Artificial Sequence
  Synthetic
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFDPHFLRVPCWKITLFVIVPVLGPLVALIICYNWLHRRLAGQFLEELRNPF

SEQ ID NO: 265 - protein, 192 aa, Artificial Sequence
  Synthetic
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGK

SEQ ID NO: 266 - protein, 277 aa, Artificial Sequence
  Synthetic
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 267 - protein, 29 aa, Artificial Sequence
  Synthetic
QLQPFPQPELPYPQPYPQQPEQPYPQPQP

SEQ ID NO: 268 - protein, 28 aa, Artificial Sequence
  Synthetic
QPYPQQPEQPYPQPQPQYSQPEQPISQQ

SEQ ID NO: 269 - protein, 28 aa, Artificial Sequence
  Synthetic
QPYPFRPEQPYPQPQPQYSQPEQPISQQ

SEQ ID NO: 270 - protein, 28 aa, Artificial Sequence
  Synthetic
PSGEGSFQPSQENPQAQGSVQPQQLPQF

SEQ ID NO: 271 - protein, 23 aa, Artificial Sequence
  Synthetic
QQFPQPEQPFPQQPEQPFPQQPQ

SEQ ID NO: 272 - protein, 25 aa, Artificial Sequence
  Synthetic
PQPQQPEQPFPQSQQPEQPFPQPEQ

SEQ ID NO: 273 - protein, 27 aa, Artificial Sequence
  Synthetic
QPFPQPEQEFPQPQQPQQSFPEQQPSL

SEQ ID NO: 274 - protein, 20 aa, Artificial Sequence
  Synthetic
PQPQQPFPEQPQQPFPEQPQ

SEQ ID NO: 275 - protein, 25 aa, Artificial Sequence
  Synthetic
PQPEQPQQPFPQSEQPQQPFPQPEQ

SEQ ID NO: 276 - protein, 27 aa, Artificial Sequence
  Synthetic
QPFPQPEQEFPQPQQPQQSFPEQEPSL

SEQ ID NO: 277 - protein, 33 aa, Artificial Sequence
  Synthetic
PTPLQPEQPFPQQPQQPQQPFPQPEQPFPWQPQ

SEQ ID NO: 278 - protein, 15 aa, Artificial Sequence
  Synthetic
QQPPFSEQEQPVLPQ

SEQ ID NO: 279 - protein, 15 aa, Artificial Sequence
  Synthetic
QGQEGYYPTSPEQPG

SEQ ID NO: 280 - protein, 24 aa, Artificial Sequence
  Synthetic
IAQQQPFPEQPQPYPEQPQPYPQQ

SEQ ID NO: 281 - protein, 15 aa, Artificial Sequence
  Synthetic
PQQPFPQPEQPFPSQ

SEQ ID NO: 282 - protein, 15 aa, Artificial Sequence
  Synthetic
PQQPFPQPEQPFPLQ

SEQ ID NO: 283 - protein, 15 aa, Artificial Sequence
  Synthetic
QFPPQPEQPFPQPQQ

SEQ ID NO: 284 - protein, 15 aa, Artificial Sequence
  Synthetic
QFPPQPEQPFPQPHQ

SEQ ID NO: 285 - protein, 15 aa, Artificial Sequence
  Synthetic
PQQPIPEQPQPYPQQ

SEQ ID NO: 286 - protein, 15 aa, Artificial Sequence
  Synthetic
SQQPIPEQPQPYPQQ

SEQ ID NO: 287 - protein, 15 aa, Artificial Sequence
  Synthetic
QPFPQPEQEFPQPQQ

SEQ ID NO: 288 - protein, 20 aa, Artificial Sequence
  Synthetic
QSIPQPEQPFPQPEQPFPQS

SEQ ID NO: 289 - protein, 15 aa, Artificial Sequence
  Synthetic
PQQPFPEQPEQIIPQ

SEQ ID NO: 290 - protein, 35 aa, Artificial Sequence
  Synthetic
SLPSCLCSFLLLLLLQVSSSYAGQFRVIGPRHPIR

SEQ ID NO: 291 - protein, 35 aa, Artificial Sequence
  Synthetic
VSSSYAGQFRVIGPRHPIRALVGDEVELPCRISPG

SEQ ID NO: 292 - protein, 35 aa, Artificial Sequence
  Synthetic
EEAAMELKVEDPFYWVSPGVLVLLAVLPVLLLQIT

SEQ ID NO: 293 - protein, 35 aa, Artificial Sequence
  Synthetic
SPGVLVLLAVLPVLLLQITVGLIFLCLQYRLRGKL

SEQ ID NO: 294 - protein, 35 aa, Artificial Sequence
  Synthetic
QITVGLIFLCLQYRLRGKLRAEIENLHRTFDPHFL

SEQ ID NO: 295 - protein, 35 aa, Artificial Sequence
  Synthetic
GKLRAEIENLHRTFDPHFLRVPCWKITLFVIVPVL

SEQ ID NO: 296 - protein, 35 aa, Artificial Sequence
  Synthetic
HFLRVPCWKITLFVIVPVLGPLVALIICYNWLHRR

SEQ ID NO: 297 - protein, 32 aa, Artificial Sequence
  Synthetic
PVLGPLVALIICYNWLHRRLAGQFLEELRNPF

SEQ ID NO: 298 - protein, 35 aa, Artificial Sequence
  Synthetic
DPGSRPHLIRLFSRDAPGREDNTFKDRPSESDELQ

SEQ ID NO: 299 - protein, 35 aa, Artificial Sequence
  Synthetic
GRTQDENPVVHFFKNIVTPRTPPPSQGKGRGLSLS

SEQ ID NO: 300 - protein, 35 aa, Artificial Sequence
  Synthetic
GGRASDYKSAHKGFKGVDAQGTLSKIFKLGGRDSR

SEQ ID NO: 301 - protein, 35 aa, Artificial Sequence
  Synthetic
FSKNYQDYEYLINVIHAFQYVIYGTASFFFLYGAL

SEQ ID NO: 302 - protein, 35 aa, Artificial Sequence
  Synthetic
AFQYVIYGTASFFFLYGALLLAEGFYTTGAVRQIF

SEQ ID NO: 303 - protein, 35 aa, Artificial Sequence
  Synthetic
GALLLAEGFYTTGAVRQIFGDYKTTICGKGLSATV

SEQ ID NO: 304 - protein, 35 aa, Artificial Sequence
  Synthetic
HPDKFVGITYALTVVWLLVFACSAVPVYIYFNTWT

SEQ ID NO: 305 - protein, 35 aa, Artificial Sequence
  Synthetic
LLVFACSAVPVYIYFNTWTTCQSIAFPSKTSASIG

SEQ ID NO: 306 - protein, 35 aa, Artificial Sequence
  Synthetic
TWTTCQSIAFPSKTSASIGSLCADARMYGVLPWNA

SEQ ID NO: 307 - protein, 35 aa, Artificial Sequence
  Synthetic
SICKTAEFQMTFHLFIAAFVGAAATLVSLLTFMIA

SEQ ID NO: 308 - protein, 35 aa, Artificial Sequence
  Synthetic
AAFVGAAATLVSLLTFMIAATYNFAVLKLMGRGTK

SEQ ID NO: 309 - protein, 20 aa, Artificial Sequence
  Synthetic
MIAATYNFAVLKLMGRGTKF

SEQ ID NO: 310 - protein, 18 aa, Artificial Sequence
  Synthetic
MAEPRQEFEVMEDHAGTY

SEQ ID NO: 311 - protein, 22 aa, Artificial Sequence
  Synthetic
VQKEQAHSEEHLGRAAFPGAPG

SEQ ID NO: 312 - protein, 16 aa, Artificial Sequence
  Synthetic
EDRDVDESSPQDSPPS

SEQ ID NO: 313 - protein, 12 aa, Artificial Sequence
  Synthetic
GQDAPLEFTFHV

SEQ ID NO: 314 - protein, 25 aa, Artificial Sequence
  Synthetic
MAEPRQEFEVMEDHAGTYGLGDRKD

SEQ ID NO: 315 - protein, 11 aa, Artificial Sequence
  Synthetic
EVMEDHAGTYG

SEQ ID NO: 316 - protein, 18 aa, Artificial Sequence
  Synthetic
MAEPRQEFEVMEDHAGTY

SEQ ID NO: 317 - protein, 24 aa, Artificial Sequence
  Synthetic
RKDQGGYTMHQDQEGDTDAGLKES

SEQ ID NO: 318 - protein, 70 aa, Artificial Sequence
  Synthetic
IFQKLNIFDQSFYDLSLQTSEIKEEEKELRRTTYKLQVKNEEVKNMSLELNSKLESLLEEKILLQQKVKY

SEQ ID NO: 319 - protein, 20 aa, Artificial Sequence
  Synthetic
HGVTSAPDTRPAPGSTAPPA

SEQ ID NO: 320 - protein, 20 aa, Artificial Sequence
  Synthetic
DTRPAPGSTAPPAHGVTSAP

SEQ ID NO: 321 - protein, 27 aa, Artificial Sequence
  Synthetic
GSTAPPAHGVTSAPDTRPAPGSTAPPA

SEQ ID NO: 322 - protein, 12 aa, Artificial Sequence
  Synthetic
GVTSAPDTRPAP

SEQ ID NO: 323 - protein, 12 aa, Artificial Sequence
  Synthetic
APDTRPAPGSTA

SEQ ID NO: 324 - protein, 14 aa, Artificial Sequence
  Synthetic
GSTAPPAHGVTSAP

SEQ ID NO: 325 - protein, 6 aa, Artificial Sequence
  Synthetic
DTRPAP

SEQ ID NO: 326 - protein, 5 aa, Artificial Sequence
  Synthetic
GSTAP

SEQ ID NO: 327 - protein, 7 aa, Artificial Sequence
  Synthetic
IAALESK

SEQ ID NO: 328 - protein, 7 aa, Artificial Sequence
  Synthetic
IAALKSK

SEQ ID NO: 329 - protein, 7 aa, Artificial Sequence
  Synthetic
IAALESE

SEQ ID NO: 330 - protein, 7 aa, Artificial Sequence
  Synthetic
IAALKSE

SEQ ID NO: 331 - protein, 7 aa, Artificial Sequence
  Synthetic
VAALKAE

SEQ ID NO: 332 - protein, 7 aa, Artificial Sequence
  Synthetic
IAALKAE

SEQ ID NO: 333 - protein, 7 aa, Artificial Sequence
  Synthetic
LAALKAE

SEQ ID NO: 334 - protein, 7 aa, Artificial Sequence
  Synthetic
VSALKAE

SEQ ID NO: 335 - protein, 7 aa, Artificial Sequence
  Synthetic
ISALKAE

SEQ ID NO: 336 - protein, 7 aa, Artificial Sequence
  Synthetic
LSALKAE

SEQ ID NO: 337 - protein, 7 aa, Artificial Sequence
  Synthetic
VASLKAE

SEQ ID NO: 338 - protein, 7 aa, Artificial Sequence
  Synthetic
IASLKAE

SEQ ID NO: 339 - protein, 7 aa, Artificial Sequence
  Synthetic
LASLKAE

SEQ ID NO: 340 - protein, 7 aa, Artificial Sequence
  Synthetic
VSSLKAE

SEQ ID NO: 341 - protein, 7 aa, Artificial Sequence
  Synthetic
ISSLKAE

SEQ ID NO: 342 - protein, 7 aa, Artificial Sequence
  Synthetic
LSSLKAE

SEQ ID NO: 343 - protein, 7 aa, Artificial Sequence
  Synthetic
VAALKSE

SEQ ID NO: 344 - protein, 7 aa, Artificial Sequence
  Synthetic
LAALKSE

SEQ ID NO: 345 - protein, 7 aa, Artificial Sequence
  Synthetic
VSALKSE

SEQ ID NO: 346 - protein, 7 aa, Artificial Sequence
  Synthetic
ISALKSE

SEQ ID NO: 347 - protein, 7 aa, Artificial Sequence
  Synthetic
LSALKSE

SEQ ID NO: 348 - protein, 7 aa, Artificial Sequence
  Synthetic
VASLKSE

SEQ ID NO: 349 - protein, 7 aa, Artificial Sequence
  Synthetic
IASLKSE

SEQ ID NO: 350 - protein, 7 aa, Artificial Sequence
  Synthetic
KSELAAI

SEQ ID NO: 351 - protein, 7 aa, Artificial Sequence
  Synthetic
KSKLAAI

SEQ ID NO: 352 - protein, 7 aa, Artificial Sequence
  Synthetic
ESSLAAI

SEQ ID NO: 353 - protein, 7 aa, Artificial Sequence
  Synthetic
ESKLAAI

SEQ ID NO: 354 - protein, 7 aa, Artificial Sequence
  Synthetic
EAKLAAV

SEQ ID NO: 355 - protein, 7 aa, Artificial Sequence
  Synthetic
EAKLAAI

SEQ ID NO: 356 - protein, 7 aa, Artificial Sequence
  Synthetic
EAKLAAL

SEQ ID NO: 357 - protein, 7 aa, Artificial Sequence
  Synthetic
EAKLASV

SEQ ID NO: 358 - protein, 7 aa, Artificial Sequence
  Synthetic
EAKLASI

SEQ ID NO: 359 - protein, 7 aa, Artificial Sequence
  Synthetic
EAKLASL

SEQ ID NO: 360 - protein, 7 aa, Artificial Sequence
  Synthetic
EAKLSAV

SEQ ID NO: 361 - protein, 7 aa, Artificial Sequence
  Synthetic
EAKLSAI

SEQ ID NO: 362 - protein, 7 aa, Artificial Sequence
  Synthetic
EAKLSAL

SEQ ID NO: 363 - protein, 7 aa, Artificial Sequence
  Synthetic
EAKLSSV

SEQ ID NO: 364 - protein, 7 aa, Artificial Sequence
  Synthetic
EAKLSSI

SEQ ID NO: 365 - protein, 7 aa, Artificial Sequence
  Synthetic
EAKLSSL

SEQ ID NO: 366 - protein, 7 aa, Artificial Sequence
  Synthetic
ESKLAAV

SEQ ID NO: 367 - protein, 7 aa, Artificial Sequence
  Synthetic
ESKLAAI

SEQ ID NO: 368 - protein, 7 aa, Artificial Sequence
  Synthetic
ESKLAAL

SEQ ID NO: 369 - protein, 7 aa, Artificial Sequence
  Synthetic
ESKLASV

SEQ ID NO: 370 - protein, 7 aa, Artificial Sequence
  Synthetic
ESKLASI

SEQ ID NO: 371 - protein, 7 aa, Artificial Sequence
  Synthetic
ESKLASL

SEQ ID NO: 372 - protein, 7 aa, Artificial Sequence
  Synthetic
ESKLSAV

SEQ ID NO: 373 - protein, 7 aa, Artificial Sequence
  Synthetic
ESKLSAI

SEQ ID NO: 374 - protein, 14 aa, Artificial Sequence
  Synthetic
IAALKSKIAAKESE

SEQ ID NO: 375 - protein, 14 aa, Artificial Sequence
  Synthetic
IAALKSKIAAKESK

SEQ ID NO: 376 - protein, 14 aa, Artificial Sequence
  Synthetic
IAALESEIAAKESK

SEQ ID NO: 377 - protein, 7 aa, Artificial Sequence
  Synthetic
IAALKSK

SEQ ID NO: 378 - protein, 7 aa, Artificial Sequence
  Synthetic
IAALRSR

SEQ ID NO: 379 - protein, 7 aa, Artificial Sequence
  Synthetic
IAALESK

SEQ ID NO: 380 - protein, 7 aa, Artificial Sequence
  Synthetic
IAALKSK

SEQ ID NO: 381 - protein, 7 aa, Artificial Sequence
  Synthetic
IAALESE

SEQ ID NO: 382 - protein, 7 aa, Artificial Sequence
  Synthetic
IAALKSE

SEQ ID NO: 383 - protein, 7 aa, Artificial Sequence
  Synthetic
VAALKAE

SEQ ID NO: 384 - protein, 7 aa, Artificial Sequence
  Synthetic
IAALKAE

SEQ ID NO: 385 - protein, 7 aa, Artificial Sequence
  Synthetic
LAALKAE

SEQ ID NO: 386 - protein, 7 aa, Artificial Sequence
  Synthetic
VSALKAE

SEQ ID NO: 387 - protein, 7 aa, Artificial Sequence
  Synthetic
ISALKAE

SEQ ID NO: 388 - protein, 7 aa, Artificial Sequence
  Synthetic
LSALKAE

SEQ ID NO: 389 - protein, 7 aa, Artificial Sequence
  Synthetic
VASLKAE

SEQ ID NO: 390 - protein, 7 aa, Artificial Sequence
  Synthetic
IASLKAE

SEQ ID NO: 391 - protein, 7 aa, Artificial Sequence
  Synthetic
LASLKAE

SEQ ID NO: 392 - protein, 7 aa, Artificial Sequence
  Synthetic
VSSLKAE

SEQ ID NO: 393 - protein, 7 aa, Artificial Sequence
  Synthetic
ISSLKAE

SEQ ID NO: 394 - protein, 7 aa, Artificial Sequence
  Synthetic
LSSLKAE

SEQ ID NO: 395 - protein, 7 aa, Artificial Sequence
  Synthetic
VAALKSE

SEQ ID NO: 396 - protein, 7 aa, Artificial Sequence
  Synthetic
LAALKSE

SEQ ID NO: 397 - protein, 7 aa, Artificial Sequence
  Synthetic
VSALKSE

SEQ ID NO: 398 - protein, 7 aa, Artificial Sequence
  Synthetic
ISALKSE

SEQ ID NO: 399 - protein, 7 aa, Artificial Sequence
  Synthetic
LSALKSE

SEQ ID NO: 400 - protein, 7 aa, Artificial Sequence
  Synthetic
VASLKSE

SEQ ID NO: 401 - protein, 7 aa, Artificial Sequence
  Synthetic
IASLKSE

SEQ ID NO: 402 - protein, 27 aa, Artificial Sequence
  Synthetic
  misc_feature (27)..(27): Azidolysine
KARDETAALLNSAVLGAAPLFVPPADX

SEQ ID NO: 403 - protein, 10 aa, Artificial Sequence
  Synthetic
  misc_feature (10)..(10): Azidolysine
AALLNSAVLX

SEQ ID NO: 404 - protein, 10 aa, Artificial Sequence
  Synthetic
  misc_feature (10)..(10): Azidolysine
ASMTNMELMX

SEQ ID NO: 405 - protein, 29 aa, Artificial Sequence
  Synthetic
  misc_feature (29)..(29): Azidolysine
GIPVHLELASMTNMELMSSIVHQQVFPTX

SEQ ID NO: 406 - protein, 34 aa, Artificial Sequence
  Synthetic
  misc_feature (34)..(34): Azidolysine
KKKVRGIPVHLELASMTNMELMSSIVHQQVFPTX

SEQ ID NO: 407 - protein, 33 aa, Artificial Sequence
  Synthetic
  misc_feature (33)..(33): Azidolysine
KKKKVRDFTGSNGDPSSPYSLHYLSPTGVNEYX

SEQ ID NO: 408 - protein, 36 aa, Artificial Sequence
  Synthetic
  misc_feature (36)..(36): Azidolysine
KKKKKVRGIPVHLELASMTNMELMSSIVHQQVFPTX

SEQ ID NO: 409 - protein, 35 aa, Artificial Sequence
  Synthetic
  misc_feature (35)..(35): Azidolysine
KKKKKKVRDFTGSNGDPSSPYSLHYLSPTGVNEYX

SEQ ID NO: 410 - protein, 26 aa, Artificial Sequence
  Synthetic
  misc_feature (25)..(25): Citrulline
  misc_feature (26)..(26): Azidolysine
MEVGWYRSPFSRVVHLYRNGKSPVXX

SEQ ID NO: 411 - protein, 29 aa, Artificial Sequence
  Synthetic
  misc_feature (29)..(29): Azidolysine
GRVLELFRAAQLANDVVLQIMELCGATRX

SEQ ID NO: 412 - protein, 18 aa, Artificial Sequence
  Synthetic
  misc_feature (17)..(17): Citrulline
  misc_feature (18)..(18): Azidolysine
AKFVAAWTLKAAASPVXX

SEQ ID NO: 413 - protein, 38 aa, Artificial Sequence
  Synthetic
  misc_feature (29)..(38): p Amino phenylalanine
GIPVHLELASMTNMELMSSIVHQQVFPTXXXXXXXXXX

SEQ ID NO: 414 - protein, 36 aa, Artificial Sequence
  Synthetic
  misc_feature (27)..(36): p Amino phenylalanine
KARDETAALLNSAVLGAAPLFVPPADXXXXXXXXXX

SEQ ID NO: 415 - protein, 36 aa, Artificial Sequence
  Synthetic
  misc_feature (27)..(36): p Amino phenylalanine
DFTGSNGDPSSPYSLHYLSPTGVNEYXXXXXXXXXX

SEQ ID NO: 416 - protein, 19 aa, Artificial Sequence
  Synthetic
  misc_feature (10)..(19): p Amino phenylalanine
ASMTNMELMXXXXXXXXXX

SEQ ID NO: 417 - protein, 19 aa, Artificial Sequence
  Synthetic
  misc_feature (10)..(19): p Amino phenylalanine
AALLNSAVLXXXXXXXXXX

SEQ ID NO: 418 - protein, 19 aa, Artificial Sequence
  Synthetic
  misc_feature (10)..(19): p Amino phenylalanine
SSPYSLHYLXXXXXXXXXX

SEQ ID NO: 419 - protein, 23 aa, Artificial Sequence
  Synthetic
  misc_feature (13)..(13): Citrulline
  misc_feature (14)..(23): p Amino phenylalanine
ASMTNMELMSPVXXXXXXXXXXX

SEQ ID NO: 420 - protein, 23 aa, Artificial Sequence
  Synthetic
  misc_feature (13)..(13): Citrulline
  misc_feature (14)..(23): p Amino phenylalanine
AALLNSAVLSPVXXXXXXXXXXX

SEQ ID NO: 421 - protein, 23 aa, Artificial Sequence
  Synthetic
  misc_feature (13)..(13): Citrulline
  misc_feature (14)..(23): p Amino phenylalanine
SSPYSLHYLSPVXXXXXXXXXXX

SEQ ID NO: 422 - protein, 33 aa, Artificial Sequence
  Synthetic
  misc_feature (29)..(33): p Amino phenylalanine
GIPVHLELASMTNMELMSSIVHQQVFPTXXXXX

SEQ ID NO: 423 - protein, 33 aa, Artificial Sequence
  Synthetic
  misc_feature (29)..(33): p Amino phenylalanine
GRVLELFRAAQLANDVVLQIMELCGATRXXXXX

SEQ ID NO: 424 - protein, 31 aa, Artificial Sequence
  Synthetic
  misc_feature (27)..(31): p Amino phenylalanine
KARDETAALLNSAVLGAAPLFVPPADXXXXX

SEQ ID NO: 425 - protein, 31 aa, Artificial Sequence
  Synthetic
  misc_feature (27)..(31): p Amino phenylalanine
DFTGSNGDPSSPYSLHYLSPTGVNEYXXXXX

SEQ ID NO: 426 - protein, 40 aa, Artificial Sequence
  Synthetic
  misc_feature (1)..(10): p Amino phenylalanine
XXXXXXXXXXGIPVHLELASMTNMELMSSIVHQQVFPTSP

SEQ ID NO: 427 - protein, 31 aa, Artificial Sequence
  Synthetic
  misc_feature (1)..(5): p Amino phenylalanine
XXXXXSKLLSFMAPIDHTTMSDDARTELFRS

SEQ ID NO: 428 - protein, 40 aa, Artificial Sequence
  Synthetic
  misc_feature (31)..(40): p Amino phenylalanine
GRVLELFRAAQLANDVVLQIMELCGATRSPXXXXXXXXXX

SEQ ID NO: 429 - protein, 44 aa, Artificial Sequence
  Synthetic
  misc_feature (43)..(43): Citrulline
  misc_feature (44)..(44): Azidolysine
KKKKKKKKKVRGIPVHLELASMTNMELMSSIVHQQVFPTSPVXX

SEQ ID NO: 430 - protein, 42 aa, Artificial Sequence
  Synthetic
  misc_feature (41)..(41): Citrulline
  misc_feature (42)..(42): Azidolysine
KKKKKKKVRGRVLELFRAAQLANDVVLQIMELCGATRSPVXX

SEQ ID NO: 431 - protein, 41 aa, Artificial Sequence
  Synthetic
  misc_feature (40)..(40): Citrulline
  misc_feature (41)..(41): Azidolysine
KKKKKKKKVRKARDETAALLNSAVLGAAPLFVPPADSPVXX

SEQ ID NO: 432 - protein, 42 aa, Artificial Sequence
  Synthetic
  misc_feature (41)..(41): Citrulline
  misc_feature (42)..(42): Azidolysine
KKKKKKKKKVRDFTGSNGDPSSPYSLHYLSPTGVNEYSPVXX

SEQ ID NO: 433 - protein, 40 aa, Artificial Sequence
  Synthetic
  misc_feature (39)..(39): Citrulline
  misc_feature (40)..(40): Azidolysine
KKKKKKKVRKARDETAALLNSAVLGAAPLFVPPADSPVXX

SEQ ID NO: 434 - protein, 43 aa, Artificial Sequence
  Synthetic
  misc_feature (42)..(42): Citrulline
  misc_feature (43)..(43): Azidolysine
KKKKKKKKKKVRVTNTEMFVTAPDNLGYMYEVQWPGQESPVXX

SEQ ID NO: 435 - protein, 43 aa, Artificial Sequence
  Synthetic
  misc_feature (29)..(29): Norleucine
  misc_feature (42)..(42): Citrulline
  misc_feature (43)..(43): Azidolysine
KKKKKKKKKKVRVTNTEMFVTAPDNLGYXYEVQWPGQESPVXX

SEQ ID NO: 436 - protein, 44 aa, Artificial Sequence; identical to SEQ ID NO: 429
  Synthetic
  misc_feature (43)..(43): Citrulline
  misc_feature (44)..(44): Azidolysine

SEQ ID NO: 437 - protein, 44 aa, Artificial Sequence
  Synthetic
  misc_feature (22)..(22): Norleucine
  misc_feature (25)..(25): Norleucine
  misc_feature (28)..(28): Norleucine
  misc_feature (43)..(43): Citrulline
  misc_feature (44)..(44): Azidolysine
KKKKKKKKKVRGIPVHLELASXTNXELXSSIVHQQVFPTSPVXX

SEQ ID NO: 438 - protein, 44 aa, Artificial Sequence; identical to SEQ ID NO: 437
  Synthetic
  misc_feature (22)..(22): Norvaline
  misc_feature (25)..(25): Norvaline
  misc_feature (28)..(28): Norvaline
  misc_feature (43)..(43): Citrulline
  misc_feature (44)..(44): Azidolysine

SEQ ID NO: 439 - protein, 44 aa, Artificial Sequence
  Synthetic
  misc_feature (43)..(43): Citrulline
  misc_feature (44)..(44): Azidolysine
KKKKKKKKKVRGIPVHLELASLTNLELLSSIVHQQVFPTSPVXX

SEQ ID NO: 440 - protein, 33 aa, Artificial Sequence
  Synthetic
  misc_feature (32)..(32): Citrulline
  misc_feature (33)..(33): Azidolysine
KKKKKKKVRVITGIKAVYNFATCGIFALSPVXX

SEQ ID NO: 441 - protein, 33 aa, Artificial Sequence
  Synthetic
  misc_feature (23)..(23): alpha Aminobutyrric acid
  misc_feature (32)..(32): Citrulline
  misc_feature (33)..(33): Azidolysine
KKKKKKKVRVITGIKAVYNFATXGIFALSPVXX

SEQ ID NO: 442 - protein, 33 aa, Artificial Sequence
  Synthetic
  misc_feature (32)..(32): Citrulline
  misc_feature (33)..(33): Azidolysine
KKKKKKKVRVITGIKAVYNFATSGIFALSPVXX

SEQ ID NO: 443 - protein, 40 aa, Artificial Sequence
  Synthetic
  misc_feature (39)..(39): Citrulline
  misc_feature (40)..(40): Azidolysine
KKKKKKVRREGVELCPGNKYEMRRHGTTHSLVIHDSPVXX

SEQ ID NO: 444 - protein, 40 aa, Artificial Sequence
  Synthetic
  misc_feature (15)..(15): alpha Aminobutyric acid
  misc_feature (39)..(39): Citrulline
  misc_feature (40)..(40): Azidolysine
KKKKKKVRREGVELXPGNKYEMRRHGTTHSLVIHDSPVXX

SEQ ID NO: 445 - protein, 34 aa, Artificial Sequence
  Synthetic
  misc_feature (33)..(33): Citrulline
  misc_feature (34)..(34): Azidolysine
KKKKKKKVRGQAEPDRAHYNIVTFCCKCDSPVXX

SEQ ID NO: 446 - protein, 34 aa, Artificial Sequence
  Synthetic
  misc_feature (25)..(26): alpha Aminobutyric acid
  misc_feature (28)..(28): alpha Aminobutyric acid
  misc_feature (33)..(33): Citrulline
  misc_feature (34)..(34): Azidolysine
KKKKKKKVRGQAEPDRAHYNIVTFXXKXDSPVXX

SEQ ID NO: 447 - protein, 26 aa, Artificial Sequence
  Synthetic
  misc_feature (1)..(1): Norleucine
  misc_feature (25)..(25): Citrulline
  misc_feature (26)..(26): Azidolysine
XEVGWYRSPFSRVVHLYRNGKSPVXX

SEQ ID NO: 448 - protein, 30 aa, Artificial Sequence
  Synthetic
  misc_feature (6)..(6): alpha Aminobutyric acid
  misc_feature (8)..(8): alpha Aminobutyric acid
  misc_feature (29)..(29): Citrulline
  misc_feature (30)..(30): Azidolysine
RLERVBHBLGKWLGHPDKFVGITYASPVXX

SEQ ID NO: 449 - protein, 31 aa, Artificial Sequence
  Synthetic
  misc_feature (31)..(31): Azidolysine
KKVRKARDETAALLNSAVLGAAPLFVPPADX

SEQ ID NO: 450 - protein, 31 aa, Artificial Sequence
  Synthetic
  misc_feature (6)..(6): Norleucine
  misc_feature (30)..(30): Citrulline
  misc_feature (31)..(31): Azidolysine
KKKVRXEVGWYRSPFSRVVHLYRNGKSPVXX

SEQ ID NO: 451 - protein, 36 aa, Artificial Sequence
  Synthetic
  misc_feature (12)..(12): alpha Aminobutyric acid
  misc_feature (14)..(14): alpha Aminobutyric acid
  misc_feature (35)..(35): Citrulline
  misc_feature (36)..(36): Azidolysine
KKKKKVRLERVBHBLGKWLGHPDKFVGITYASPVXX

SEQ ID NO: 452 - protein, 29 aa, Artificial Sequence
  Synthetic
  misc_feature (28)..(28): Citrulline
  misc_feature (29)..(29): Azidolysine
SLKISQAVHAAHAEINEAGREVVGSPVXX

SEQ ID NO: 453 - protein, 35 aa, Artificial Sequence
  Synthetic
  misc_feature (34)..(34): Citrulline
  misc_feature (35)..(35): Azidolysine
KKKVRESLKISQAVHAAHAEINEAGREVVGSPVXX

SEQ ID NO: 454 - protein, 40 aa, Artificial Sequence
  Synthetic
  misc_feature (39)..(39): Citrulline
  misc_feature (40)..(40): Azidolysine
KKKKKKKKVRESLKISQAVHAAHAEINEAGREVVGSPVXX

SEQ ID NO: 455 - protein, 33 aa, Artificial Sequence
  Synthetic
  misc_feature (33)..(33): Azidolysine
KKKKVRKARDETAALLNSAVLGAAPLFVPPADX

SEQ ID NO: 456 - protein, 34 aa, Artificial Sequence
  Synthetic
IAALKSKIAALKSKGGTESNKKFLPFQQFGRDIA

SEQ ID NO: 457 - protein, 14 aa, Artificial Sequence
  Synthetic
IAALESEIAALESE

SEQ ID NO: 458 - protein, 18 aa, Artificial Sequence
  Synthetic
  misc_feature (17)..(17): Citrulline
  misc_feature (18)..(18): Azidolysine
AKFVAAWTLKAAASPVXX

SEQ ID NO: 459 - protein, 33 aa, Artificial Sequence
  Synthetic
GGGSGGGGSGGGGSGTESNKKFLPFQQFGRDIA

SEQ ID NO: 460 - protein, 34 aa, Artificial Sequence
  Synthetic
APAPAPAPAPAPAPGGTESNKKFLPFQQFGRDIA

SEQ ID NO: 461 - protein, 34 aa, Artificial Sequence
  Synthetic
IAALESKIAALESKGGTESNKKFLPFQQFGRDIA

SEQ ID NO: 462 - protein, 32 aa, Artificial Sequence
  Synthetic
IAALESKIAALESKTESNKKFLPFQQFGRDIA

SEQ ID NO: 463 - protein, 34 aa, Artificial Sequence; identical to SEQ ID NO: 461
  Synthetic

SEQ ID NO: 464 - protein, 14 aa, Artificial Sequence
  Synthetic
IAALESKIAALESK

SEQ ID NO: 465 - protein, 18 aa, Artificial Sequence
  Synthetic
TESNKKFLPFQQFGRDIA

SEQ ID NO: 466 - protein, 34 aa, Artificial Sequence
  Synthetic
IAALESKIAALESKAPTESNKKFLPFQQFGRDIA

SEQ ID NO: 467 - protein, 38 aa, Artificial Sequence
  Synthetic
IAALESKIAALESKAPAPAPTESNKKFLPFQQFGRDIA

SEQ ID NO: 468 - protein, 34 aa, Artificial Sequence; identical to SEQ ID NO: 461
  Synthetic

SEQ ID NO: 469 - protein, 48 aa, Artificial Sequence
  Synthetic
IAALESKIAALESKIAALESKIAALESKGGTESNKKFLPFQQFGRDIA

SEQ ID NO: 470 - protein, 34 aa, Artificial Sequence
  Synthetic
KSELAAIKSELAAIGGTESNKKFLPFQQFGRDIA

SEQ ID NO: 471 - protein, 30 aa, Artificial Sequence
  Synthetic
  misc_feature (30)..(30): Azidolysine
SIPWNLERITPPRGGESKIAALESKIAALX

SEQ ID NO: 472 - protein, 30 aa, Artificial Sequence
  Synthetic
  misc_feature (30)..(30): Azidolysine
SIPWNLERITPPRGGIAALESKIAALESKX

SEQ ID NO: 473 - protein, 28 aa, Artificial Sequence
  Synthetic
  misc_feature (28)..(28): Azidolysine
SIPWNLERITPPRESKIAALESKIAALX

SEQ ID NO: 474 - protein, 30 aa, Artificial Sequence
  Synthetic
  misc_feature (30)..(30): Azidolysine
SIPWNLERITPPRGGKSELAAIKSELAAIX

SEQ ID NO: 475 - protein, 29 aa, Artificial Sequence
  Synthetic
IAALESKIAALESKGGPGFLVKMSGDLLE

SEQ ID NO: 476 - protein, 37 aa, Artificial Sequence
  Synthetic
IAALESKIAALESKGGQDNSSFDSLSPEPKSRFAMLD

SEQ ID NO: 477 - protein, 35 aa, Artificial Sequence
  Synthetic
IAALESKIAALESKQDNSSFDSLSPEPKSRFAMLD

SEQ ID NO: 478 - protein, 36 aa, Artificial Sequence
  Synthetic
  misc_feature (36)..(36): Azidolysine
QDNSSFDSLSPEPKSRFAMLDESKIAALESKIAALX

SEQ ID NO: 479 - protein, 26 aa, Artificial Sequence
  Synthetic
IAALESKIAALESKGGEPKSRFAMLD

SEQ ID NO: 480 - protein, 33 aa, Artificial Sequence
  Synthetic
RFAMLDDVKILANGLLQLGHGLKDFVHKTKGQI

SEQ ID NO: 481 - protein, 49 aa, Artificial Sequence
  Synthetic
IAALESKIAALESKGGRFAMLDDVKILANGLLQLGHGLKDFVHKTKGQI

SEQ ID NO: 482 - protein, 49 aa, Artificial Sequence
  Synthetic
  misc_feature (32)..(32): Citrulline
  misc_feature (49)..(49): Azidolysine
GRVLELFRAAQLANDVVLQIMELCGATRSPVXGGESKIAALESKIAALX

SEQ ID NO: 483 - protein, 49 aa, Artificial Sequence
  Synthetic
  misc_feature (32)..(32): Citrulline
  misc_feature (49)..(49): Azidolysine
GIPVHLELASMTNMELMSSIVHQQVFPTSPVXGGESKIAALESKIAALX

SEQ ID NO: 484 - protein, 41 aa, Artificial Sequence
  Synthetic
  misc_feature (16)..(17): alpha Aminobutyric acid
  misc_feature (19)..(19): alpha Aminobutyric acid
  misc_feature (24)..(24): Citrulline
  misc_feature (41)..(41): Azidolysine
GQAEPDRAHYNIVTFBBKBDSPVXGGESKIAALESKIAALX

SEQ ID NO: 485 - protein, 47 aa, Artificial Sequence
  Synthetic
  misc_feature (30)..(30): Citrulline
  misc_feature (47)..(47): Azidolysine
KARDETAALLNSAVLGAAPLFVPPADSPVXGGESKIAALESKIAALX

SEQ ID NO: 486 - protein, 41 aa, Artificial Sequence
  Synthetic
IAALESKIAALESKGGPSKPSKRSFIEDLLFNKVTLADAGF

SEQ ID NO: 487 - protein, 36 aa, Artificial Sequence
  Synthetic
IAALESKIAALESKGGARDLICAQKFNGLTVLPPLL

SEQ ID NO: 488 - protein, 35 aa, Artificial Sequence
  Synthetic
IAALESKIAALESKGGLYNSASFSTFKCYGVSPTK

SEQ ID NO: 489 - protein, 28 aa, Artificial Sequence
  Synthetic
IAALESKIAALESKGGVYAWNRKRISNC

SEQ ID NO: 490 - protein, 38 aa, Artificial Sequence
  Synthetic
IAALESKIAALESKGGLVYENQKLIANQFNSAIGKIQD

SEQ ID NO: 491 - protein, 39 aa, Artificial Sequence
  Synthetic
IAALESKIAALESKGGFNSAIGKIQDSLSSTASALGKLQ

SEQ ID NO: 492 - protein, 40 aa, Artificial Sequence
  Synthetic
IAALESKIAALESKGGPADGNPDPNANPNVDPNANPNVDP

SEQ ID NO: 493 - protein, 36 aa, Artificial Sequence
  Synthetic
IAALESKIAALESKGGNANPNANPNANPNANPNANP

SEQ ID NO: 494 - protein, 40 aa, Artificial Sequence
  Synthetic
IAALESKIAALESKGGNANPNVDPNANPNVDPNANPNVDP

SEQ ID NO: 495 - protein, 13 aa, Artificial Sequence
  Synthetic
  misc_feature (12)..(12): Citrulline
  misc_feature (13)..(13): Azidolysine
RAHYNIVTFSVXX

SEQ ID NO: 496 - protein, 10 aa, Artificial Sequence
  Synthetic
RAHYNIVTFS

SEQ ID NO: 497 - protein, 12 aa, Artificial Sequence
  Synthetic
  misc_feature (2)..(2): Citrulline
VXRAHYNIVTFS

SEQ ID NO: 498 - protein, 16 aa, Artificial Sequence
  Synthetic
  misc_feature (1)..(4): p Amino phenylalanine
  misc_feature (6)..(6): Citrulline
XXXXVXRAHYNIVTFS

SEQ ID NO: 499 - protein, 18 aa, Artificial Sequence
  Synthetic
  misc_feature (1)..(4): p Amino phenylalanine
  misc_feature (8)..(8): Citrulline
XXXXSPVXRAHYNIVTFS

SEQ ID NO: 500 - protein, 12 aa, Artificial Sequence
  Synthetic
  misc_feature (2)..(2): Citrulline
VXRAHYNIVTFS

SEQ ID NO: 501 - protein, 14 aa, Artificial Sequence
  Synthetic
KKKKKRAHYNIVTF

SEQ ID NO: 502 - protein, 9 aa, Artificial Sequence
  Synthetic
RAHYNIVTF

SEQ ID NO: 503 - protein, 12 aa, Artificial Sequence
  Synthetic
KVRRAHYNIVTF

SEQ ID NO: 504 - protein, 16 aa, Artificial Sequence
  Synthetic
KKKKKVRRAHYNIVTF

SEQ ID NO: 505 - protein, 20 aa, Artificial Sequence
  Synthetic
  misc_feature (20)..(20): Citrulline
KKKKKVRRAHYNIVTFSPVX

SEQ ID NO: 506 - protein, 22 aa, Artificial Sequence
  Synthetic
  misc_feature (22)..(22): Citrulline
KKKKKVRRAHYNIVTFGGSPVX

SEQ ID NO: 507 - protein, 17 aa, Artificial Sequence
  Synthetic
  misc_feature (6)..(6): b Alanine
KKKKKXVRRAHYNIVTF

SEQ ID NO: 508 - protein, 18 aa, Artificial Sequence
  Synthetic
KKKKKSPVRRAHYNIVTF

SEQ ID NO: 509 - protein, 18 aa, Artificial Sequence
  Synthetic
  misc_feature (9)..(9): alpha Aminobutyric acid
KKKKKSPVXRAHYNIVTF

SEQ ID NO: 510 - protein, 18 aa, Artificial Sequence
  Synthetic
  misc_feature (9)..(9): Citrulline
KKKKKSPVXRAHYNIVTF

SEQ ID NO: 511 - protein, 18 aa, Artificial Sequence
  Synthetic
  misc_feature (9)..(9): alpha Aminobutyric acid
KKKKKSKVXRAHYNIVTF

SEQ ID NO: 512 - protein, 18 aa, Artificial Sequence
  Synthetic
  misc_feature (9)..(9): alpha Aminobutyric acid
KKKKKSKSXRAHYNIVTF

SEQ ID NO: 513 - protein, 18 aa, Artificial Sequence
  Synthetic
KKKKKSKVRRAHYNIVTF

SEQ ID NO: 514 - protein, 18 aa, Artificial Sequence
  Synthetic
KKKKKSKSRRAHYNIVTF

SEQ ID NO: 515 - protein, 20 aa, Artificial Sequence
  Synthetic
GQAEPDRAHYNIVTFCCKCD

SEQ ID NO: 516 - protein, 12 aa, Artificial Sequence
  Synthetic
  misc_feature (11)..(11): Citrulline
  misc_feature (12)..(12): p Amino phenylalanine
AQLANDVVLVXX

SEQ ID NO: 517 - protein, 9 aa, Artificial Sequence
  Synthetic
AQLANDVVL

SEQ ID NO: 518 - protein, 11 aa, Artificial Sequence
  Synthetic
  misc_feature (2)..(2): Citrulline
VXAQLANDVVL

SEQ ID NO: 519 - protein, 16 aa, Artificial Sequence
  Synthetic
  misc_feature (6)..(6): Citrulline
  misc_feature (7)..(7): p Amino phenylalanine
FFFFVZXAQLANDVVL

SEQ ID NO: 520 - protein, 17 aa, Artificial Sequence
  Synthetic
  misc_feature (1)..(4): p Amino phenylalanine
  misc_feature (8)..(8): Citrulline
XXXXSPVXAQLANDVVL

SEQ ID NO: 521 - protein, 11 aa, Artificial Sequence
  Synthetic
  misc_feature (2)..(2): Citrulline
VXAQLANDVVL

SEQ ID NO: 522 - protein, 32 aa, Artificial Sequence
  Synthetic
  misc_feature (21)..(21): Norleucine
  misc_feature (24)..(24): alpha Aminobutyric acid
GRVLELFRAAQLANDVVLQIXELBGATRSPVR

SEQ ID NO: 523 - protein, 36 aa, Artificial Sequence
  Synthetic
  misc_feature (21)..(21): Norleucine
  misc_feature (24)..(24): alpha Aminobutyric acid
GRVLELFRAAQLANDVVLQIXELBGATRKKKKKKKK

SEQ ID NO: 524 - protein, 40 aa, Artificial Sequence
  Synthetic
  misc_feature (21)..(21): b Alanine
  misc_feature (24)..(24): alpha Aminobutyric acid
  misc_feature (33)..(33): alpha Aminobutyric acid
GRVLELFRAAQLANDVVLQIXELBGATRSPVRXKKKKKKK

SEQ ID NO: 525 - protein, 14 aa, Artificial Sequence
  Synthetic
KKKKKAQLANDVVL

SEQ ID NO: 526 - protein, 10 aa, Artificial Sequence
  Synthetic
RAQLANDVVL

SEQ ID NO: 527 - protein, 12 aa, Artificial Sequence
  Synthetic
KVRAQLANDVVL

SEQ ID NO: 528 - protein, 16 aa, Artificial Sequence
  Synthetic
KKKKKVRAQLANDVVL

SEQ ID NO: 529 - protein, 20 aa, Artificial Sequence
  Synthetic
  misc_feature (20)..(20): Citrulline
KKKKKVRAQLANDVVLSPVX

SEQ ID NO: 530 - protein, 22 aa, Artificial Sequence
  Synthetic
  misc_feature (22)..(22): Citrulline
KKKKKVRAQLANDVVLGGSPVX

SEQ ID NO: 531 - protein, 17 aa, Artificial Sequence
  Synthetic
  misc_feature (6)..(6): b Alanine
KKKKKXVRAQLANDVVL

SEQ ID NO: 532 - protein, 18 aa, Artificial Sequence
  Synthetic
KKKKKSPVRAQLANDVVL

SEQ ID NO: 533 - protein, 18 aa, Artificial Sequence
  Synthetic
  misc_feature (9)..(9): alpha Aminobutyric acid
KKKKKSPVXAQLANDVVL

SEQ ID NO: 534 - protein, 18 aa, Artificial Sequence
  Synthetic
  misc_feature (9)..(9): Citrulline
KKKKKSPVXAQLANDVVL

SEQ ID NO: 535 - protein, 18 aa, Artificial Sequence
  Synthetic
  misc_feature (9)..(9): alpha Aminobutyric acid
KKKKKSKVXAQLANDVVL

SEQ ID NO: 536 - protein, 18 aa, Artificial Sequence
  Synthetic
  misc_feature (9)..(9): alpha Aminobutyric acid
KKKKKSKSXAQLANDVVL

SEQ ID NO: 537 - protein, 18 aa, Artificial Sequence
  Synthetic
KKKKKSKVRAQLANDVVL

SEQ ID NO: 538 - protein, 18 aa, Artificial Sequence
  Synthetic
KKKKKSKSRAQLANDVVL

SEQ ID NO: 539 - protein, 28 aa, Artificial Sequence
  Synthetic
GRVLELFRAAQLANDVVLQIMELCGATR

SEQ ID NO: 540 - protein, 5 aa, Artificial Sequence
  Synthetic
VTSAP